(19) United States
(12) Patent Application Publication
(10) Pub. No.: US 2025/0059187 A1
(43) Pub. Date: Feb. 20, 2025

(54) COVALENTLY BINDING INHIBITORS OF G12S, G12D AND/OR G12E MUTANTS OF K-RAS GTPASE

(71) Applicant: The Regents of the University of California, Oakland, CA (US)

(72) Inventors: Kevan M. Shokat, San Francisco, CA (US); Ziyang Zhang, San Francisco, CA (US); Qinheng Zheng, San Francisco, CA (US)

(21) Appl. No.: 18/720,571

(22) PCT Filed: Dec. 21, 2022
(86) PCT No.: PCT/US2022/082136

§ 371 Date: Jun. 14, 2024

Related U.S. Application Data
(60) Provisional application No. 63/292,910, filed on Dec. 22, 2021.
(60) Provisional application No. 63/313,040, filed on Feb. 23, 2022.
(60) Provisional application No. 63/322,528, filed on Mar. 22, 2022.

(51) Int. Cl. C07D 471/04; A61K 31/519; C07D 519/00

(52) U.S. Cl. CPC C07D 471/04; A61K 31/519; C07D 519/00

(57) ABSTRACT

Described herein, inter alia, are GTPase inhibitors and uses thereof.

### CROSS-REFERENCES TO RELATED APPLICATIONS

This application claims the benefit of U.S. Provisional Application No. 63/322,528, filed Mar. 22, 2022, U.S. Provisional Application No. 63/313,040, filed Feb. 23, 2022, and U.S. Provisional Application No. 63/292,910, filed Dec. 22, 2021, which are incorporated herein by reference in their entirety and for all purposes.

### REFERENCE TO AN ELECTRONIC SEQUENCE LISTING

The contents of the electronic sequence listing (048536-724001WO_Sequence_Listing_ST26.xml; Size: 75,428 bytes; and Date of Creation: Dec. 14, 2022) is hereby incorporated by reference in its entirety.

### BACKGROUND

Drugs that directly impede the function of driver oncogenes offer exceptional efficacy and therapeutic window for the treatment of cancer. The recently approved mutant-selective small molecule cysteine reactive covalent inhibitor of the G12C mutant of K-Ras, sotorasib, provides a case in point. KRAS is the most frequently mutated protooncogene in human cancer, yet despite the success targeting the G12C allele, targeted therapy for other hotspot mutants of KRAS have not been described. Oncogenic mutations of Ras are one of the most common genetic alterations in human cancer, with an estimated disease burden of >3 million patients per year worldwide. In the KRAS gene, a GGT to GAT nucleotide transition at codon 12 (c.35 G>A) gives rise to KRAS G12D, the most frequent Ras mutation accounting for about 23% of Ras-driven cancers. Selective targeting of KRAS G12D while sparing wild type KRAS is a highly desirable therapeutic goal pursued by many research groups, as it would enable a large therapeutic window for cancer treatment. Disclosed herein, inter alia, are solutions to these and other problems in the art.

### BRIEF SUMMARY

In an aspect is provided a compound, or a pharmaceutically acceptable salt thereof, having the formula:

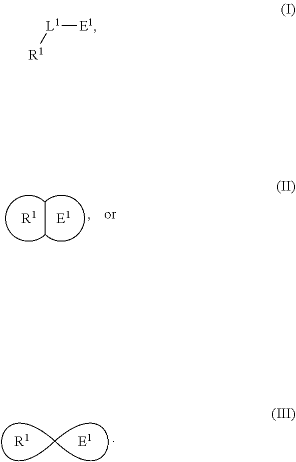

R¹ is a Switch II Binding Pocket binding moiety.
L¹ is a bond or divalent linker.
E¹ is an electrophilic moiety capable of forming a covalent bond with a Switch II GTPase protein serine residue or a Switch II GTPase protein threonine residue.

In an aspect is provided a compound, or a pharmaceutically acceptable salt thereof, having the formula:

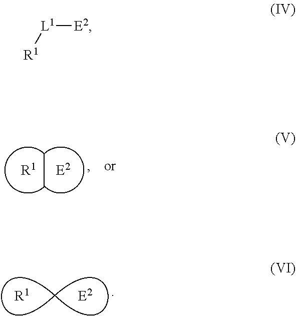

R¹ is a Switch II Binding Pocket binding moiety.
L¹ is a bond or divalent linker.
E² is an electrophilic moiety capable of forming a covalent bond with a Switch II GTPase protein aspartate residue or a Switch II GTPase protein glutamate residue.

In an aspect is provided a pharmaceutical composition including a compound described herein, or a pharmaceutically acceptable salt thereof, and a pharmaceutically acceptable excipient.

In an aspect is provided a method of treating cancer in a subject in need thereof, the method including administering to the subject in need thereof a therapeutically effective amount of a compound described herein, or a pharmaceutically acceptable salt thereof.

In an aspect is provided a method of treating a K-Ras(G12S)-associated disease in a subject in need thereof, the method including administering to the subject in need thereof a therapeutically effective amount of a compound described herein, or a pharmaceutically acceptable salt thereof.

In an aspect is provided a method of treating an H-Ras(G12S)-associated disease in a subject in need thereof, the method including administering to the subject in need thereof a therapeutically effective amount of a compound described herein, or a pharmaceutically acceptable salt thereof.

In an aspect is provided a method of treating an N-Ras(G12S)-associated disease in a subject in need thereof, the method including administering to the subject in need thereof a therapeutically effective amount of a compound described herein, or a pharmaceutically acceptable salt thereof.

In an aspect is provided a method of treating a K-Ras(G12D)-associated disease in a subject in need thereof, the method including administering to the subject in need thereof a therapeutically effective amount of a compound described herein, or a pharmaceutically acceptable salt thereof.

In an aspect is provided a method of treating an H-Ras(G12D)-associated disease in a subject in need thereof, the method including administering to the subject in need thereof a therapeutically effective amount of a compound described herein, or a pharmaceutically acceptable salt thereof.

In an aspect is provided a method of treating an N-Ras(G12D)-associated disease in a subject in need thereof, the method including administering to the subject in need thereof a therapeutically effective amount of a compound described herein, or a pharmaceutically acceptable salt thereof.

In an aspect is provided a method of modulating the level of activity of a K-Ras protein in a cell, the method including contacting the cell with an effective amount of a compound described herein, or a pharmaceutically acceptable salt thereof.

In an aspect is provided a method of modulating the level of activity of an H-Ras protein in a cell, the method including contacting the cell with an effective amount of a compound described herein, or a pharmaceutically acceptable salt thereof.

In an aspect is provided a method of modulating the level of activity of an N-Ras protein in a cell, the method including contacting the cell with an effective amount of a compound described herein, or a pharmaceutically acceptable salt thereof.

In an aspect is provided a K-Ras protein covalently bound to a compound described herein, or a pharmaceutically acceptable salt thereof, wherein the compound is covalent bound to a serine residue of the K-Ras protein.

In an aspect is provided an H-Ras protein covalently bound to a compound described herein, or a pharmaceutically acceptable salt thereof, wherein the compound is covalent bound to a serine residue of the H-Ras protein.

In an aspect is provided an N-Ras protein covalently bound to a compound described herein, or a pharmaceutically acceptable salt thereof, wherein the compound is covalent bound to a serine residue of the N-Ras protein.

In an aspect is provided a K-Ras protein covalently bound to a compound described herein, or a pharmaceutically acceptable salt thereof, wherein the compound is covalent bound to an aspartate residue of the K-Ras protein.

In an aspect is provided an H-Ras protein covalently bound to a compound described herein, or a pharmaceutically acceptable salt thereof, wherein the compound is covalent bound to an aspartate residue of the H-Ras protein.

In an aspect is provided an N-Ras protein covalently bound to a compound described herein, or a pharmaceutically acceptable salt thereof, wherein the compound is covalent bound to an aspartate residue of the N-Ras protein.

### BRIEF DESCRIPTION OF THE DRAWINGS

FIGS. 1A-1C. FIG. 1A: Immunoblot of Ba/F3 cells expressing wild-type K-Ras, K-Ras(G12S) or K-Ras(G12C). IL-3 was removed from the culture media 10 minutes before cells were lysed and analyzed. Data is representative of two experiments using independently generated Ba/F3 transductants. FIG. 1B: Growth of Ba/F3 transductants in the absence of IL-3. Data is representative of two experiments using independently generated Ba/F3 transductants. Error bars represent standard deviations. FIG. 1C: Intrinsic and NF1-mediated single-turnover GTPase activity of wild-type K-Ras (circles), K-Ras(G12S) (squares), and K-Ras(G12C) (triangles) (n=3). Error bars represent standard deviations.

FIGS. 2A-2G. FIG. 2A: Structures of β-lactones 1′ and 2′. FIG. 2B: Covalent modification of 4 μM recombinant K-Ras(G12S)·GDP or wild-type K-Ras·GDP proteins treated with 10 μM 1′ (first bar in each set of two) or 2′ (second bar in each set of two) assessed by whole protein mass spectrometry (n=3). FIG. 2C: Differential scanning fluorimetry of unmodified K-Ras(G12S)·GDP (black) and the K-Ras(G12S)·GDP·1′ complex (grey) (n=3). FIG. 2D: Illustration of a biochemical assay that monitors nucleotide exchange using a fluorescent-GDP analog. FIG. 2E: Nucleotide exchange of wildtype K-Ras (circles), K-Ras(G12S) (squares), or K-Ras(G12S)·1′ adduct (triangles) in the presence of magnesium (intrinsic), Sos, or EDTA (n=3). FIG. 2F: X-ray co-crystal structure of the K-Ras(G12S)·GDP·1′ complex. 2Fₒ−F_{c} map for the ligand 1′ and serine 12 is depicted in grey mesh (1.0 σ). FIG. 2G: Scheme illustration of the nucleophilic ring opening of the β-lactone in 1′ by serine 12.

FIGS. 3A-3F. FIG. 3A: Structures of K-Ras(G12S) ligands 3′-5′. FIG. 3B: Time-dependent covalent modification of recombinant K-Ras(G12S)·GDP protein by 10 μM compound (ADA: circles; compound 3′: squares; compound 4′: triangles tip up; compound 5′: triangles tip down) at 23° C. assessed by whole protein mass spectrometry (n=3, replicates are plotted as individual data points). FIG. 3C: Immunoblot of A549 cells treated with 10 μM adagrasib, 3′, 4′, or 5′ for 2 h. Data is representative of two independent experiments. FIG. 3D: Immunoblot of A549 cells treated with various concentrations of 5′ for 2 h. Data is representative of two independent experiments. FIG. 3E: Immunoblot of A549, A375, SW1990, and H358 cells treated with DMSO or 10 μM 5′ for 2 h. Data is representative of two independent experiments. FIG. 3F: Relative growth of Ba/F3 parental cells (+10 ng/mL IL-3) (circles) and Ba/F3:K-Ras(G12S) cells (no IL-3) (squares) after treatment with adagrasib or 5′ for 72 h. Data is representative of three independent experiments.

FIG. 4. Measurement of the Kᵢ and k_{inact} for the reaction between K-Ras(G12S)·GDP and compound 1′.

FIG. 5. Compound 1′ does not react with GppNHp-loaded K-Ras(G12S).

FIGS. 6A-6C. Comparison of the structure of the K-Ras(G12S)·GDP·compound 1′ adduct with reported crystal structures of K-Ras(G12C)·GDP bound by electrophilic ligands. FIG. 6A: Crystal structure of the K-Ras(G12S)·GDP·compound 1′ adduct. FIG. 6B: Crystal structure of the K-Ras(G12C)·GDP·adagrasib adduct (PDB: 6USZ). FIG. 6C: Superimposition of the conformations of covalent ligands of the Switch II pocket: compound 1′, adagrasib (PDB: 6USZ), sotorasib (PDB: 60IM), ARS1620 (PDB: 5V9U).

FIG. 7. Phospho-ERK levels of BaF3 parental cells (+10 ng/mL IL-3) (circles) and BaF3/K-Ras(G12S) cells (no IL-3) (squares) after treatment with adagrasib or 5′ for 1 h.

FIG. 8. Sanger sequencing of the KRAS Exon 2 of G12S-mutant cell lines. Genomic DNA sequence is presented as the antisense strand. Arrow indicates the c. 34G>A mutation. A549 and KMS20 were determined to carry homozygous KRAS p. G12S mutation, and HKA-1 and LS123 were determined to carry heterozygous KRAS p. G12S mutation. Sequences: K-Ras GASGVGKS (residues 10-17 of SEQ ID NO:4) or GAGGVGKS (residues 10-17 of SEQ ID NO:1); gDNA(anti-sense): ACTCTTGCCTACGCCACTAGCTCCA (SEQ ID NO:5).

FIG. 9. Validation of the mutant-specific Ras(G12S) antibody. A pan-Ras antibody (abeam 108602) and a mutant-specific Ras(G12S) antibody (NewEastBio 26186) were used to detect recombinant K-Ras(wildtype), K-Ras(G12S) and K-Ras(G12S)·1′ adduct.

FIG. 10. Uncropped immunoblot images for FIG. 1A.

FIG. 11. Uncropped immunoblot images for FIG. 3C.

FIG. 12. Uncropped immunoblot images for FIG. 3D. Note: Due to limitations of gel size (12-well), the samples (1-10 and 11-14) were run on two separate gels and transferred onto a single membrane in a single transfer sandwich. Some bands have vertical offsets between the two gels due to technical difficulties of perfectly aligning gels during the transfer.

FIG. 13. Examples of GTPases containing serine at position 12 or equivalent (i.e., corresponding to position 12): human KRas (UniProt P01116): MTEYKLVVVGASGVGKSALTIQLIQNHFVDEYDPTIEDSYRKQVVIDGETCLLDILDT AGQEEYSA (residues 1-66 of SEQ ID NO:4), human ERas (UniProt Q7Z444): MELPTKPGTFDLGLATWSPSFQGETHRAQARRRDVGRQLPEYKAVVVGASGVGKS ALTIQLNHQCFVEDHDPTIQDSYWKELTLDSGDCILNVLDTAGQAIHRA (SEQ ID NO:6), human RASD1 (UniProt Q9Y272): MKLAAMIKKMCPSDSELSIPAKNCYRMVILGSSKVGKTAIVSRFLTGRFEDAYTPTIE DFHRKFYSIRGEVYQLDILDTSGNHPFPA (SEQ ID NO:7), human Rhes (UniProt Q96D21): MMKTLSSGNCTLSVPAKNSYRMVVLGASRVGKSSIVSRFLNGRFEDQYTPTIEDFHR KVYNIRGDMYQLDILDTSGNHPFPA (SEQ ID NO:8), human RASL11B (UniProt Q9BPW5): MRLIQNMCTIAEYPAPGNAAASDCCVGAAGRRLVKIAVVGASGVGKTALVVRFLTK RFIGDYERNAGNLYTRQVQIEGETLALQVQDTPGIQVHENSL (SEQ ID NO:9), human REM2 (UniProt Q8IYK8): GAPRRRGSMPVPYKHQLRRAQAVDELDWPPQASSSGSSDSLGSGEAAPAQKDGIFK VMLVGESGVGKSTLAGTFGGLQGDSAHEPENPEDTYERRIMVDKEEVTLVVYDIWE QGD (SEQ ID NO:10). Only partial sequence alignment is shown. K-Ras sequence (RASK_HUMAN) is shown as the G12S mutant.

FIG. 14. Examples of GTPases containing serine at position 12 or equivalent (i.e., corresponding to position 12): human KRas (UniProt P01116): MTEYKLVVVGASGVGKSALTIQLIQNHFVDEYDPTIEDSYRKQVVIDGETCLLDILDT AGQEEYSAMRDQYMRTGEGFLCVFAINNTKSFEDIHHYREQ (residues 1-99 of SEQ ID NO:4), human RHOH (UniProt Q15669): MLSSIKCVLVGDSAVGKTSLLVRFTSETFPEAYKPTVYENTGVDVFMDGIQISLGLW DTAGNDAFRSIRPLSYQQADVVLMCYSVANHNSFLNLKNKWIGE (SEQ ID NO:11), human RND3 (UniProt P61587): MKERRASQKLSSKSIMDPNQNVKCKIVVVGDSQCGKTALLHVFAKDCFPENYVPTV FENYTASFEIDTQRIELSLWDTSGSPYYDNVRPLSYPDSDAVLICFDISRPETLDSVLK KWKGE (SEQ ID NO:12). Only partial sequence alignment is shown. K-Ras sequence (RASK_HUMAN) is shown as the G12S mutant.

FIG. 15. Examples of GTPases containing serine at position 12 or equivalent (i.e., corresponding to position 12): human KRas (UniProt P01116): MTEYKLVVVGASGVGKSALTIQLIQNHFVDEYDPTIEDSYRKQVVIDGETCLLDILDT AGQEEYSAMRDQYMRTGEGFLCVFAINNTKSFEDIHHYREQIKRVKDSEDVPM (residues 1-111 of SEQ ID NO:4), human RAB1A (UniProt P62820): MSSMNPEYDYLFKLLLIGDSGVGKSCLLLRFADDTYTESYISTIGVDFKIRTIELDGKT IKLQIWDTAGQERFRTITSSYYRGAHGIIVVYDVTDQESFNNVKQWLQEIDRYASEN VNK (SEQ ID NO:13), human RAB1B (UniProt Q9H0U4): MNPEYDYLFKLLLIGDSGVGKSCLLLRFADDTYTESYISTIGVDFKIRTIELDGKTIKL QIWDTAGQERFRTITSSYYRGAHGIIVVYDVTDQESYANVKQWLQEIDRYASENVNK (SEQ ID NO:14), human RAB2A (UniProt P61019): MAYAYLFKYIIIGDTGVGKSCLLLQFTDKRFQPVHDLTIGVEFGARMITIDGKQIKLQI WDTAGQESFRSITRSYYRGAAGALLVYDITRRDTFNHLTTWLEDARQHSNSNMVI (SEQ ID NO:15), human RAB2B (UniProt Q8WUD1): MTYAYLFKYIIIGDTGVGKSCLLLQFTDKRFQPVHDLTIGVEFGARMVNIDGKQIKLQ IWDTAGQESFRSITRSYYRGAAGALLVYDITRRETFNHLTSWLEDARQHSSSNMVI (SEQ ID NO:16). Only partial sequence alignment is shown. K-Ras sequence (RASK_HUMAN) is shown as the G12S mutant.

FIG. 16. Examples of GTPases containing serine at position 12 or equivalent (i.e., corresponding to position 12): human KRas (UniProt P01116): MTEYKLVVVGASGVGKSALTIQLIQNHFVDEYDPTIEDSYRKQVVIDGETCLLDILDT AGQEEYSAMRDQYMRTGEGFLCVFAINNTKSFEDIHHYREQIKRVKDSEDVP (residues 1-110 of SEQ ID NO:4), human GNAZ (UniProt P19086): MGCRQSSEEKEAARRSRRIDRHLRSESQRQRREIKLLLLGTSNSGKSTIVKQMKIIHSG GFNLEACKEYKPLIIYNAIDSLTRIIRALAALRIDFHNPDRAYDAVQLFALTGPAESKG EITPELLGVMRRLWADPGAQACFSRSSEYHLEDNAAYYLNDLERIAAADYIP (SEQ ID NO:17). Only partial sequence alignment is shown. K-Ras sequence (RASK_HUMAN) is shown as the G12S mutant.

FIG. 17. Examples of GTPases containing serine at position 12 or equivalent (i.e., corresponding to position 12): human KRas (UniProt P01116): MTEYKLVVVGASGVGKSALTIQLIQNHFVDEYDPTIEDSYRKQVVIDGETCLLDILDT AGQEEYSAMRDQYMRTGEGFLCVFAINNTKSFEDIHHYREQIKRVKDSEDVPMVLV GNKCDLPSRTVDTKQAQDLARSYGIPFIETSAKTRQR (SEQ ID NO:4), human LRRK2 (UniProt Q5S007): FLQQRLKKAVPYNRMKLMIVGNTGSGKTTLLQQLMKTKKSDLGMQSATVGIDVKD WPIQIRDKRKRDLVLNWDFAGREEFYSTHPHFMTQRALYLAVYDLSKGQAEVDAM KPWLFNIKARASSSPVILVGTHLDVSDEKQRKACMSKITKELLNKRGFPAIRDYHFVN AT (SEQ ID NO:18). Only partial sequence alignment is shown. K-Ras sequence (RASK_HUMAN) is shown as the G12S mutant.

FIGS. 18A-18C. β-propiolactone covalently modifies the mutant aspartate in K-Ras(G12D). FIG. 18A: Schematic showing the reaction between an aspartate residue and β-propiolactone. FIG. 18B: MS/MS spectrum of β-propiolactone-modified K-Ras(G12D) peptide. Sequence: LVVVGADGVGK (SEQ ID NO:19). FIG. 18C: Intensity of the modified peptide shown in FIG. 18B under different treatment conditions.

FIGS. 19A-19H. Compound 1 is a selective covalent ligand of K-Ras(G12D). FIG. 19A: chemical structure of β-lactone compound 1. FIG. 19B: Covalent modification of recombinant K-Ras proteins by 10 μM compound 1 at 23° C. FIG. 19C: Time-dependent covalent modification of K-Ras(G12D)·GDP and K-Ras(G12D)·GppNHp by 10 μM compound 1 at 23° C. FIG. 19D: Differential scanning fluorimetry for K-Ras(G12D)·GDP, K-Ras(G12D)·GppNHp and their covalent adducts with compound 1. FIG. 19E: Binding of GST-Raf1-RBD to immobilized K-Ras(G12D)·GppNHp and K-Ras(G12D)·GppNHp·1 measured by biolayer interferometry. FIG. 19F: Intact protein mass spectra of K-Ras(G12D) with or without (RS)-1 treatment. FIG. 19G: Kinetics of covalent modification of K-Ras(G12D) by (RS)-1. FIG. 19H: Compound (RS)-1 disrupted Ras-RafRBD binding.

FIGS. 20A-20F. The reaction between compound 1 and K-Ras(G12D) is stereoselective. FIG. 20A: Crystal structure of the covalent adduct between K-Ras(G12D)·GDP and compound 1. FIG. 20B: Fo−Fc omit map of the ligand and Asp12, contoured at 2.0 σ. FIG. 20C: Chemical structure of the adduct formed between the ligand and Asp12 after the opening of the β-lactone ring. FIG. 20D: Two possible reaction pathways leading to the observed stereochemistry of the adduct. FIG. 20E: Chemical structures of enantiomerically pure (S)-2 and (R)-2. FIG. 20F: Time-dependent covalent modification of K-Ras(G12D)·GDP by 10 μM (S)-2 or 10 μM (R)-2.

FIGS. 21A-21C. Compound 1, also referred to as 14-049, inhibits p-ERK signaling in KRAS G12D mutant cells. FIG. 21A: Growth inhibition of BaF3/K-Ras(G12D) cells by compound 1 in the presence or absence of IL-3. FIG. 21B: Compound 1 inhibited growth of KRAS^{G12D} cancer cell lines. FIG. 21C: Growth inhibition of K-Ras(G12D) cell lines by compound 1.

FIGS. 22A-22B. Covalent adduct formation stabilizes K-Ras(G12D) in both GDP- and GTP-states. Differential scanning fluorimetry for K-Ras(G12D)·GDP, K-Ras(G12D)·GppNHp and their covalent adducts with compound 14-005. FIG. 22A: β-Lactone 14-005 stabilized K-Ras(G12D)·GDP. FIG. 22B: β-Lactone 14-005 stabilized K-Ras(G12D)·GppNHp.

FIG. 23. β-lactones covalently modify K-Ras(G12D). Time-dependent covalent modification of K-Ras(G12D)·GDP and K-Ras(G12D)·GppNHp by 10 μM 14-005, 14-036, and 14-049 at 23° C.

FIG. 24. Compound 1, also referred to as 14-049, inhibits the proliferation of K-Ras(G12D) mutant cells. Growth inhibition of AsPc-1 and SW1990 cells by 14-005, 14-036, and 14-049.

FIG. 25. Inhibition of the interaction between K-Ras and Raf1-RBD by compound 1.

FIGS. 26A-26H. FIG. 26A: Chemical structure of substituted β-lactones. FIG. 26B: Time-dependent covalent modification of K-Ras(G12D)·GDP. FIG. 26C: Stability of substituted β-lactones in PBS 7.4 at 23° C. FIG. 26D: Growth inhibition of BaF3/K-Ras(G12D) cells by substituted β-lactones. FIG. 26E: Chemical structures of the two enantiomers of compound 4. FIG. 26F: Time-dependent covalent modification of K-Ras(G12D)·GDP. FIG. 26G: Growth inhibition of BaF3/K-Ras(G12D) cells by each enantiomer of compound 4. FIG. 26H: Inhibition of K-Ras(G12D) signaling in SW1990 cells.

FIG. 27. Examples of GTPases containing aspartate at position 12 or equivalent (i.e., corresponding to position 12): ARF1 (e.g., UniProt P84077): MGNIFANLFKGLFGKKEMRILMVGLDAAGKTTILYKLKLG (SEQ ID NO:20), ARF3 (e.g., UniProt P61204): MGNIFGNLLKSLIGKKEMRILMVGLDAAGKTTILYKLKLG (SEQ ID NO:21), ARF4 (e.g., UniProt P18085): MGLTISSLFSRLFGKKQMRILMVGLDAAGKTTILYKLKLG (SEQ ID NO:22), ARF5 (e.g., UniProt P84085): MGLTVSALFSRIFGKKQMRILMVGLDAAGKTTILYKLKLG (SEQ ID NO:23), ARF6 (e.g., UniProt P62330): MGKVLSKIFGNKEMRILMLGLDAAGKTTILYKLKLG (SEQ ID NO:24), TRIM23 (e.g., UniProt P36406): MAFTKDNRVHIGPKMEIRVVTLGLDGAGKTTILFKLKQD (SEQ ID NO:25), ARL1 (e.g., UniProt P40616): MGGFFSSIFSSLFGTREMRILILGLDGAGKTTILYRLQVG (SEQ ID NO:26), ARL2 (e.g., UniProt P36404): MGLLTILKKMKQKERELRLLMLGLDNAGKTTILKK (SEQ ID NO:27), ARL3 (e.g., UniProt P36405): MGLLSILRKLKSAPDQEVRILLLGLDNAGKTTLLKQ (SEQ ID NO:28), ARL4A (e.g., UniProt P40617): MGDQTSILSNLPSFQSFHIVILGLDCAGKTTVLYRLQFN (SEQ ID NO: 29), ARL4B: MGDQTSILSSLPSFQSFHIVMLGLDCAGKTTVLYRLQFN (SEQ ID NO:30), ARL5: MGILFTRIWRLFNHQEHKVIIVGLDNAGKTTILYQFSMN (SEQ ID NO:31), ARL6 (e.g., UniProt Q9H0F7): MGLLDRLSVLLGLKKKEVHVLCLGLDNSGKTTIINKLKPSN (SEQ ID NO:32), ARL7: MGNISSNISAFQSLHIVMLGLDSAGKTTVLYRLKFN (SEQ ID NO:33), ARL8: MGLIFAKLWSLFCNQEHKVIIVGLDNAGKTTILYQFLMN (SEQ ID NO:34), ARL9 (e.g., UniProt Q6T311): MRWKALSHPAWPEEKNKQILVLGLDGAGKTSVLHSLASN (SEQ ID NO:35), ARL12: MGQLIAKLMSIFGNQEHTVIIVGLDNEGKTTILYRFLTN (SEQ ID NO:36), ARF4L: MGMAPTASSFLPHFQALHVVVIGLDSAGKTSLLYRLKFK (SEQ ID NO:37), ARL11 (e.g., UniProt Q969Q4): MGSVNSRGHKAEAQVVMMGLDSAGKTTLLYKLKGH (SEQ ID NO:38), ARF7: MGSLGSKNPQTKQAQVLLLGLDSAGKSTLLYKLKLA (SEQ ID NO:39), 339231 (e.g., UniProt QPN6): MCLLLGATGVGKTLLVKRLQEV (SEQ ID NO:40), DKFZp761: MFCCGWFKRWREPVRKVTLLMVGLDNAGKTATAKGIQGE (SEQ ID NO:41), ARFRP1 (e.g., UniProt Q13795): MYTLLSGLYKYMFQKDEYCILILGLDNAGKTTFLEQSKTRF (SEQ ID NO:42), ARFRP2 (e.g., UniProt Q9NXU5): MSRALCCKGPPPARPEYDLVCIGLTGSGKTSLLSKLCSE (SEQ ID NO:43), ARL10A (e.g., UniProt Q8N8L6): MADEEDEEPALEELEQREVLVLGLDGAGKSTFLRVLSGKP (SEQ ID NO:44), ARL10B (e.g., UniProt Q96BM9): MIKLLDWFKALFWKEEMELTLVGLQYSGKTTFVNVIASGQ (SEQ ID NO:45), ARL10C: MLRLLDWFRSLFWKEEMELTLVGLQYSGKTTFVNVIASGQ (SEQ ID NO:46), 344988: MSFSSVPQFLGLNKKSGKLLFVGLNNTDKTILLHMIKDD (SEQ ID NO:47), SARA1 (e.g., UniProt Q6FID4): MSFSSVLQFLGLYKKSGKLVFLGLDNAGKTTLLHMLKDD (SEQ ID NO:48), SARA2: MSFSSVLQFLGLYKKTGKLVFLGLDNAGKTTLLHMLKDD (SEQ ID NO:49). Only partial sequence alignment is shown.

FIGS. 28A-28C. FIG. 28A: Chemical structures of (2R, 3S)-4, (2R, 3S)-5, and (2R, 3S)-6. FIG. 28B: GTP-state reactive β-lactone K-Ras(G12D) inhibitors. FIG. 28B: Pseudo-first-order kinetics of K-Ras(G12D) labeling by isopropyl-substituted β-lactones. FIG. 28C: Western blot time-course are consistent with observed recombinant K-Ras(G12D) labeling kinetics.

FIGS. 29A-29H. FIG. 29A: Chemical structure of compound (R)-7. FIG. 29B: Labeling kinetics of recombinant K-Ras(G12D) with (R)-7. FIG. 29C: Second-order kinetics of recombinant K-Ras(G12D) with (R)-7. FIG. 29D: Compound (R)-7 selectively modified recombinant K-Ras(G12D) in both nucleotide states. FIG. 29E: Compound (R)-7 selectively modified cellular K-Ras(G12D) and inhibited signaling pathways. FIG. 29F: Compound (R)-7 potently inhibited Ba/F3 KRAS G12D cell line growth via on-target inhibition of K-Ras(G12D). FIG. 29G: Compound (R)-7 selectively inhibited growth of cancer cells lines harboring K-Ras(G12D) mutation. FIG. 29H: The difference between Compound (R)-7 induced apoptosis was significant between K-Ras(G12D) cell lines and non-K-Ras(G12D) cell lines.

FIG. 30. Compound 6 fully labels K-Ras(G12D) in cell at submicrimolar concentrations.

FIG. 31. Selective covalent engagement of K-Ras(G12D) in cells for compound 6.

FIGS. 32A-32B. Full in-cell covalent labeling can be achieved by repeatedly dosing with as low as 40 nM of compound 6. FIG. 32A: Time course of (2R,3S)-6-induced in-cell K-Ras(G12D) labeling and downstream signaling pathway inhibition. FIG. 32B: Medium replacement enabled complete labeling of endogenous K-Ras(G12D) by (2R,3S)-6 at 40 nM.

FIGS. 33A-33C. On-target growth inhibition assessed in BaF3/K-Ras(G12D) cells. FIG. 33A: Growth inhibition of Ba/F3 KRAS G12D cells by (2R,3S)-5. FIG. 33B: Growth inhibition of Ba/F3 KRAS G12D cells by (2R,3S)-6. FIG. 33C: Growth inhibition of Ba/F3 KRAS G12D cells by (RS)-7.

FIG. 34. Stereospecific synthesis of β-lactone K-Ras(G12D) warheads.

FIG. 35. One example of a synthetic method of compound 7.

### DETAILED DESCRIPTION

### I. Definitions

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di-, and multivalent radicals. The alkyl may include a designated number of carbons (e.g., C₁-C₁₀ means one to ten carbons). In embodiments, the alkyl is fully saturated. In embodiments, the alkyl (e.g., C₁-C₁₀, C₁-C₆, or C₁-C₄ alkyl) is fully saturated. In embodiments, the alkyl is monounsaturated. In embodiments, the alkyl is polyunsaturated. Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. In embodiments, an alkyl moiety may be an alkenyl moiety (e.g., C₂-C₁₀ alkenyl, C₂-C₆ alkenyl, or C₂-C₄ alkenyl). An alkyl moiety may be an alkynyl moiety. In embodiments, an alkyl moiety may be an alkynyl moiety (e.g., C₂-C₁₀ alkynyl, C₂-C₆ alkynyl, or C₂-C₄ alkynyl). An alkenyl includes one or more double bonds. An alkynyl includes one or more triple bonds.

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene. The term “alkynylene” by itself or as part of another substituent, means, unless otherwise stated, a divalent radical derived from an alkyne. In embodiments, the alkylene is fully saturated. In embodiments, the alkylene is monounsaturated. In embodiments, the alkylene is polyunsaturated. An alkenylene includes one or more double bonds. An alkynylene includes one or more triple bonds.

The term “heteroalkyl,” by itself or in combination with another term, means, unless otherwise stated, a stable straight or branched chain, or combinations thereof, including at least one carbon atom and at least one heteroatom (e.g., O, N, P, Si, and S), and wherein the nitrogen and sulfur atoms may optionally be oxidized, and the nitrogen heteroatom may optionally be quaternized. The heteroatom(s) (e.g., N, S, Si, or P) may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —S—CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P). The term “heteroalkenyl,” by itself or in combination with another term, means, unless otherwise stated, a heteroalkyl including at least one double bond. A heteroalkenyl may optionally include more than one double bond and/or one or more triple bonds in additional to the one or more double bonds. The term “heteroalkynyl,” by itself or in combination with another term, means, unless otherwise stated, a heteroalkyl including at least one triple bond. A heteroalkynyl may optionally include more than one triple bond and/or one or more double bonds in additional to the one or more triple bonds. In embodiments, the heteroalkyl is fully saturated. In embodiments, the heteroalkyl is monounsaturated. In embodiments, the heteroalkyl is polyunsaturated.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like. The term “heteroalkenylene,” by itself or as part of another substituent, means, unless otherwise stated, a divalent radical derived from a heteroalkene. The term “heteroalkynylene” by itself or as part of another substituent, means, unless otherwise stated, a divalent radical derived from a heteroalkyne. In embodiments, the heteroalkylene is fully saturated. In embodiments, the heteroalkylene is monounsaturated. In embodiments, the heteroalkylene is polyunsaturated. A heteroalkenylene includes one or more double bonds. A heteroalkynylene includes one or more triple bonds.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. In embodiments, the heterocycloalkyl is hexahydro-1H-pyrrolizin-7a-yl. In embodiments, the heterocycloalkyl is 2-pyrrolidinyl. A “cycloalkylene” and a “heterocycloalkylene,” alone or as part of another substituent, means a divalent radical derived from a cycloalkyl and heterocycloalkyl, respectively. In embodiments, the cycloalkyl is fully saturated. In embodiments, the cycloalkyl is monounsaturated. In embodiments, the cycloalkyl is polyunsaturated. In embodiments, the heterocycloalkyl is fully saturated. In embodiments, the heterocycloalkyl is monounsaturated. In embodiments, the heterocycloalkyl is polyunsaturated.

In embodiments, the term “cycloalkyl” means a monocyclic, bicyclic, or a multicyclic cycloalkyl ring system. In embodiments, monocyclic ring systems are cyclic hydrocarbon groups containing from 3 to 8 carbon atoms, where such groups can be saturated or unsaturated, but not aromatic. In embodiments, cycloalkyl groups are fully saturated. A bicyclic or multicyclic cycloalkyl ring system refers to multiple rings fused together wherein at least one of the fused rings is a cycloalkyl ring and wherein the multiple rings are attached to the parent molecular moiety through any carbon atom contained within a cycloalkyl ring of the multiple rings.

In embodiments, a cycloalkyl is a cycloalkenyl. The term “cycloalkenyl” is used in accordance with its plain ordinary meaning. In embodiments, a cycloalkenyl is a monocyclic, bicyclic, or a multicyclic cycloalkenyl ring system. A bicyclic or multicyclic cycloalkenyl ring system refers to multiple rings fused together wherein at least one of the fused rings is a cycloalkenyl ring and wherein the multiple rings are attached to the parent molecular moiety through any carbon atom contained within a cycloalkenyl ring of the multiple rings.

In embodiments, the term “heterocycloalkyl” means a monocyclic, bicyclic, or a multicyclic heterocycloalkyl ring system. In embodiments, heterocycloalkyl groups are fully saturated. In embodiments, heterocycloalkyl (e.g., 3 to 8 membered, 3 to 6 membered, 4 to 6 membered, 4 to 5 membered, 5 to 6 membered, or 5 to 8 membered) groups are fully saturated. A bicyclic or multicyclic heterocycloalkyl ring system refers to multiple rings fused together wherein at least one of the fused rings is a heterocycloalkyl ring and wherein the multiple rings are attached to the parent molecular moiety through any atom contained within a heterocycloalkyl ring of the multiple rings.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring and wherein the multiple rings are attached to the parent molecular moiety through any carbon atom contained within an aryl ring of the multiple rings. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring and wherein the multiple rings are attached to the parent molecular moiety through any atom contained within a heteroaromatic ring of the multiple rings). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzoxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be —O— bonded to a ring heteroatom nitrogen.

Spirocyclic rings are two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g., substituents for cycloalkyl or heterocycloalkyl rings). Spirocylic rings may be substituted or unsubstituted cycloalkyl, substituted or unsubstituted cycloalkylene, substituted or unsubstituted heterocycloalkyl or substituted or unsubstituted heterocycloalkylene and individual rings within a spirocyclic ring group may be any of the immediately previous list, including having all rings of one type (e.g., all rings being substituted heterocycloalkylene wherein each ring may be the same or different substituted heterocycloalkylene). When referring to a spirocyclic ring system, heterocyclic spirocyclic rings means a spirocyclic rings wherein at least one ring is a heterocyclic ring and wherein each ring may be a different ring. When referring to a spirocyclic ring system, substituted spirocyclic rings means that at least one ring is substituted and each substituent may optionally be different.

The symbol “ [symbol] ” denotes the point of attachment of a chemical moiety to the remainder of a molecule or chemical formula.

The term “oxo,” as used herein, means an oxygen that is double bonded to a carbon atom.

The term “alkylarylene” as an arylene moiety covalently bonded to an alkylene moiety (also referred to herein as an alkylene linker). In embodiments, the alkylarylene group has the formula:

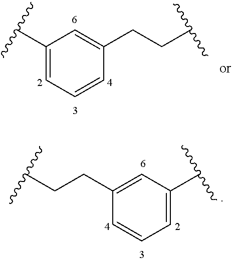 [SMILES: *CCc1cccc(*)c1 ; *CCc1cccc(*)c1]

An alkylarylene moiety may be substituted (e.g., with a substituent group) on the alkylene moiety or the arylene linker (e.g., at carbons 2, 3, 4, or 6) with halogen, oxo, —N₃, —CF₃, —CCl₃, —CBr₃, —CI₃, —CN, —CHO, —OH, —NH₂, —COOH, —CONH₂, —NO₂, —SH, —SO₂CH₃, —SO₃H, —OSO₃H, —SO₂NH₂, —NHNH₂, —ONH₂, —NHC(O)NHNH₂, substituted or unsubstituted C₁-C₅ alkyl or substituted or unsubstituted 2 to 5 membered heteroalkyl). In embodiments, the alkylarylene is unsubstituted.

Each of the above terms (e.g., “alkyl,” “heteroalkyl,” “cycloalkyl,” “heterocycloalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, halogen, —SiR′R″R′″, —OC(O)R′, —C(O)R′, —CO₂R′, —CONR′R″, —OC(O)NR′R″, —NR″C(O)R′, —NR′C(O)NR″R′″, —NR″C(O)₂R′, —NRC(NR′R″R′″)=NR″″, —NRC(NR′R″)=NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —NR′SO₂R″, —NR′C(O)R″, —NR′C(O)OR″, —NR′OR″, in a number ranging from zero to (2m′+1), where m′ is the total number of carbon atoms in such radical. R, R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound described herein includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, halogen, —SiR′R″R′″, —OC(O)R′, —C(O)R′, —CO₂R′, —CONR′R″, —OC(O)NR′R″, —NR″C(O)R′, —NR′C(O)NR″R′″, —NR″C(O)₂R′, —NR—C(NR′R″R′″)=NR″″, —NR—C(NR′R″)=NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —R′, —N₃, —CH(Ph)₂, fluoro(C₁-C₄)alkoxy, and fluoro(C₁-C₄)alkyl, —NR′SO₂R″, —NR′C(O)R″, —NR′C(O)OR″, —NR′OR″, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound described herein includes more than one R group, for example, each of the R groups is independently selected as are each R′, R″, R′″, and R″″ groups when more than one of these groups is present.

Substituents for rings (e.g., cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more floating substituents (including, but not limited to, points of attachment to the remainder of the molecule), the floating substituents may be bonded to the heteroatoms. Where the ring heteroatoms are shown bound to one or more hydrogens (e.g., a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′—(C″R″R′″)_{d}—, where s and d are independently integers of from 0 to 3, and X′ is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur (S), phosphorus (P), selenium (Se), and silicon (Si). In embodiments, the terms “heteroatom” or “ring heteroatom” are meant to include oxygen (O), nitrogen (N), sulfur (S), phosphorus (P), and silicon (Si).

A “substituent group,” as used herein, means a group selected from the following moieties:
(A) oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, —SF₅, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
(B) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from:
   (i) oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, —SF₅, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
   (ii) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from:
      (a) oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCI₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, —SF₅, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
      (b) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from: oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, —SF₅, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted phenyl, and each substituted or unsubstituted heteroaryl is a substituted or unsubstituted 5 to 6 membered heteroaryl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In some embodiments of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In some embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 9 membered heteroaryl. In some embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 9 membered heteroarylene. In some embodiments, the compound is a chemical species set forth in the Examples section, figures, or tables below.

In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is unsubstituted (e.g., is an unsubstituted alkyl, unsubstituted heteroalkyl, unsubstituted cycloalkyl, unsubstituted heterocycloalkyl, unsubstituted aryl, unsubstituted heteroaryl, unsubstituted alkylene, unsubstituted heteroalkylene, unsubstituted cycloalkylene, unsubstituted heterocycloalkylene, unsubstituted arylene, and/or unsubstituted heteroarylene, respectively). In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is substituted (e.g., is a substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene, respectively).

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, wherein if the substituted moiety is substituted with a plurality of substituent groups, each substituent group may optionally be different. In embodiments, if the substituted moiety is substituted with a plurality of substituent groups, each substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one size-limited substituent group, wherein if the substituted moiety is substituted with a plurality of size-limited substituent groups, each size-limited substituent group may optionally be different. In embodiments, if the substituted moiety is substituted with a plurality of size-limited substituent groups, each size-limited substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one lower substituent group, wherein if the substituted moiety is substituted with a plurality of lower substituent groups, each lower substituent group may optionally be different. In embodiments, if the substituted moiety is substituted with a plurality of lower substituent groups, each lower substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted moiety is substituted with a plurality of groups selected from substituent groups, size-limited substituent groups, and lower substituent groups; each substituent group, size-limited substituent group, and/or lower substituent group may optionally be different. In embodiments, if the substituted moiety is substituted with a plurality of groups selected from substituent groups, size-limited substituent groups, and lower substituent groups; each substituent group, size-limited substituent group, and/or lower substituent group is different.

In a recited claim or chemical formula description herein, each R substituent or L linker that is described as being “substituted” without reference as to the identity of any chemical moiety that composes the “substituted” group (also referred to herein as an “open substitution” on an R substituent or L linker or an “openly substituted” R substituent or L linker), the recited R substituent or L linker may, in embodiments, be substituted with one or more first substituent groups as defined below.

The first substituent group is denoted with a corresponding first decimal point numbering system such that, for example, R¹ may be substituted with one or more first substituent groups denoted by R^{1.1}, R² may be substituted with one or more first substituent groups denoted by R^{2.1}, R³ may be substituted with one or more first substituent groups denoted by R^{3.1}, R⁴ may be substituted with one or more first substituent groups denoted by R^{4.1}, R⁵ may be substituted with one or more first substituent groups denoted by R^{5.1}, and the like up to or exceeding an R¹⁰⁰ that may be substituted with one or more first substituent groups denoted by R¹⁰⁰. As a further example, R^{1A} may be substituted with one or more first substituent groups denoted by R^{1A.1}, R^{2A} may be substituted with one or more first substituent groups denoted by R^{2A.1}, R^{3A} may be substituted with one or more first substituent groups denoted by R^{3A.1}, R^{4A} may be substituted with one or more first substituent groups denoted by R^{4A.1}, R^{5A} may be substituted with one or more first substituent groups denoted by R^{5A.1} and the like up to or exceeding an R^{100A} may be substituted with one or more first substituent groups denoted by R^{100A.1}. As a further example, L¹ may be substituted with one or more first substituent groups denoted by R^{L1.1}, L² may be substituted with one or more first substituent groups denoted by R^{L2.1}, L³ may be substituted with one or more first substituent groups denoted by R^{L3.1}, L⁴ may be substituted with one or more first substituent groups denoted by R^{L4.1}, L⁵ may be substituted with one or more first substituent groups denoted by R^{L5.1} and the like up to or exceeding an L¹⁰⁰ which may be substituted with one or more first substituent groups denoted by R^{L100.1}. Thus, each numbered R group or L group (alternatively referred to herein as R^{WW} or L^{WW} wherein “WW” represents the stated superscript number of the subject R group or L group) described herein may be substituted with one or more first substituent groups referred to herein generally as R^{WW.1} or R^{LWW.1}, respectively. In turn, each first substituent group (e.g., R^{1.1}, R^{2.1}, R^{3.1}, R⁴¹, R^{5.1} . . . R^{100.1}; R^{1A.1}, R^{2A.1}, R^{3A.1}, R^{4A.1}, R^{5A.1} . . . R^{100A.1}; R^{L1.1}, R^{L2.1}, R^{L3.1}, R^{L4.1}, R^{L5.1} . . . R^{L100.1}) may be further substituted with one or more second substituent groups (e.g., R^{1.2}, R^{2.2}, R^{3.2}, R^{4.2}, R^{5.2} . . . R^{100.2}; R^{1A.2}, R^{2A.2}, R^{3A.2}, R^{4A.2}, R^{5A.2} . . . R^{100A.2}; R^{L1.2}, R^{L2.2}, R^{L3.2}, R^{L4.2}, R^{L5.2} . . . R^{L100.2}, respectively). Thus, each first substituent group, which may alternatively be represented herein as R^{WW.1} as described above, may be further substituted with one or more second substituent groups, which may alternatively be represented herein as R^{WW.2}.

Finally, each second substituent group (e.g., R^{1.2}, R^{2.2}, R^{3.2}, R^{4.2}, R^{5.2} . . . R^{100.2}; R^{1A.2}, R^{2A.2}, R^{3A.2}, R^{4A.2}, R^{5A.2} . . . R^{100A.2}; R^{L1.2}, R^{L2.2}, R^{L3.2}, R^{L4.2}, R^{L5.2} . . . R^{L100.2}) may be further substituted with one or more third substituent groups (e.g., R^{1.3}, R^{2.3}, R^{3.3}, R^{4.3}, R^{5.3} . . . R^{100.3}; R^{1A.3}, R^{2A.3}, R^{3A.3}, R^{4A.3}, R^{5A.3} . . . R^{1A.3}; R^{L1.3}, R^{L2.3}, R^{L3.3}, R^{L4.3}, R^{L5.3} . . . R^{L100.3}; respectively). Thus, each second substituent group, which may alternatively be represented herein as R^{WW.2} as described above, may be further substituted with one or more third substituent groups, which may alternatively be represented herein as R^{WW.3}. Each of the first substituent groups may be optionally different. Each of the second substituent groups may be optionally different. Each of the third substituent groups may be optionally different.

Thus, as used herein, R^{WW} represents a substituent recited in a claim or chemical formula description herein which is openly substituted. “WW” represents the stated superscript number of the subject R group (1, 2, 3, 1A, 2A, 3A, 1B, 2B, 3B, etc.). Likewise, L^{WW} is a linker recited in a claim or chemical formula description herein which is openly substituted. Again, “WW” represents the stated superscript number of the subject L group (1, 2, 3, 1A, 2A, 3A, 1B, 2B, 3B, etc.). As stated above, in embodiments, each R^{WW} may be unsubstituted or independently substituted with one or more first substituent groups, referred to herein as R^{WW.1}; each first substituent group, R^{WW.1}, may be unsubstituted or independently substituted with one or more second substituent groups, referred to herein as R^{WW.2}; and each second substituent group may be unsubstituted or independently substituted with one or more third substituent groups, referred to herein as R^{WW.3}. Similarly, each L^{WW} linker may be unsubstituted or independently substituted with one or more first substituent groups, referred to herein as R^{LWW.1}; each first substituent group, R^{LWW.1}, may be unsubstituted or independently substituted with one or more second substituent groups, referred to herein as R^{LWW.2}; and each second substituent group may be unsubstituted or independently substituted with one or more third substituent groups, referred to herein as R^{LWW.3}. Each first substituent group is optionally different. Each second substituent group is optionally different. Each third substituent group is optionally different. For example, if R^{WW} is phenyl, the said phenyl group is optionally substituted by one or more R^{WW.1} groups as defined herein below, e.g., when R^{WW.1} is R^{WW.2}-substituted or unsubstituted alkyl, examples of groups so formed include but are not limited to itself optionally substituted by 1 or more R^{WW.2}, which R^{WW.2} is optionally substituted by one or more R^{WW.3}. By way of example when the R^{WW} group is phenyl substituted by R^{WW.1}, which is methyl, the methyl group may be further substituted to form groups including but not limited to:

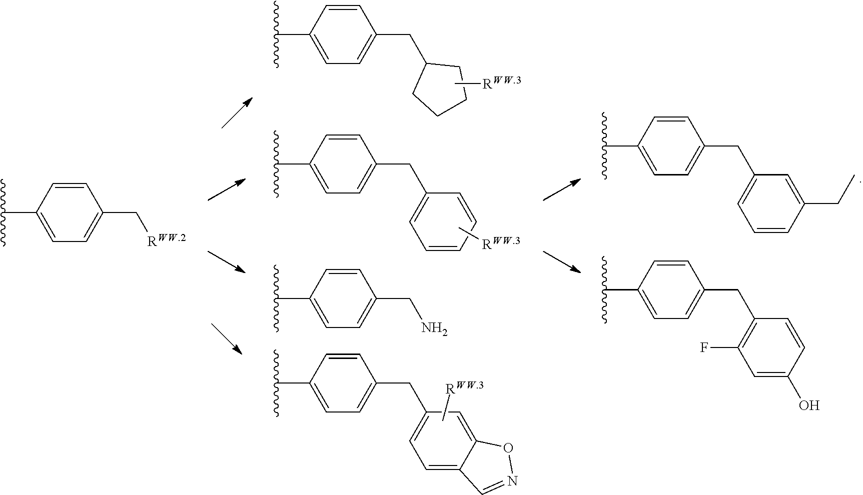

R^{WW.1} is independently oxo, halogen, —CX^{WW.1}₃, —CHX^{WW.1}₂, —CH₂X^{WW.1}, —OCX^{WW.1}₃, —OCH₂X^{WW.1}, —OCHX^{WW.1}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, R^{WW.2}-substituted or unsubstituted alkyl (e.g., C₁-C₈, C₁-C₆, C₁-C₄, or C₁-C₂), R^{WW.2}-substituted or unsubstituted heteroalkyl (e.g., 2 to 8 membered, 2 to 6 membered, 4 to 6 membered, 2 to 3 membered, or 4 to 5 membered), R^{WW.2}-substituted or unsubstituted cycloalkyl (e.g., C₃-C₈, C₃-C₆, C₄-C₆, or C₅-C₆), R^{WW.2}-substituted or unsubstituted heterocycloalkyl (e.g., 3 to 8 membered, 3 to 6 membered, 4 to 6 membered, 4 to 5 membered, or 5 to 6 membered), R^{WW.2}-substituted or unsubstituted aryl (e.g., C₆-C₁₂, C₆-C₁₀, or phenyl), or R^{WW.2}-substituted or unsubstituted heteroaryl (e.g., 5 to 12 membered, 5 to 10 membered, 5 to 9 membered, or 5 to 6 membered). In embodiments, R^{WW.1} is independently oxo, halogen, —CX^{WW.1}₃, —CHX^{WW.1}₂, —CH₂X^{WW.1}, —OCX^{WW.1}₃, —OCH₂X^{WW.1}, —OCHX^{WW.1}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{WW.1} is independently —F, —Cl, —Br, or —I.

R^{WW.2} is independently oxo, halogen, —CX^{WW.2}₃, —CHX^{WW.2}₂, —CH₂X^{WW.2}, —OCX^{WW.2}₃, —OCH₂X^{WW.2}, —OCHX^{WW.2}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, R^{WW.3}-substituted or unsubstituted alkyl (e.g., C₁-C₈, C₁-C₆, C₁-C₄, or C₁-C₂), R^{WW.3}-substituted or unsubstituted heteroalkyl (e.g., 2 to 8 membered, 2 to 6 membered, 4 to 6 membered, 2 to 3 membered, or 4 to 5 membered), R^{WW.3}-substituted or unsubstituted cycloalkyl (e.g., C₃-C₈, C₃-C₆, C₄-C₆, or C₅-C₆), R^{WW.3}-substituted or unsubstituted heterocycloalkyl (e.g., 3 to 8 membered, 3 to 6 membered, 4 to 6 membered, 4 to 5 membered, or 5 to 6 membered), R^{WW.3}-substituted or unsubstituted aryl (e.g., C₆-C₁₂, C₆-C₁₀, or phenyl), or R^{WW.3}-substituted or unsubstituted heteroaryl (e.g., 5 to 12 membered, 5 to 10 membered, 5 to 9 membered, or 5 to 6 membered). In embodiments, R^{WW.2} is independently oxo, halogen, —CX^{WW.2}₃, —CHX^{WW.2}₂, —CH₂X^{WW.2}, —OCX^{WW.2}₃, —OCH₂X^{WW.2}, —OCHX^{WW.2}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{WW.2} is independently —F, —Cl, —Br, or —I.

R^{WW.3} is independently oxo, halogen, —CX^{WW.3}₃, —CHX^{WW.3}₂, —CH₂X^{WW.3}, —OCX^{WW.3}₃, —OCH₂X^{WW.3}, —OCHX^{WW.2}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{WW.3} is independently —F, —Cl, —Br, or —I.

Where two different R^{WW} substituents are joined together to form an openly substituted ring (e.g., substituted cycloalkyl, substituted heterocycloalkyl, substituted aryl or substituted heteroaryl), in embodiments the openly substituted ring may be independently substituted with one or more first substituent groups, referred to herein as R^{WW.1}; each first substituent group, R^{WW.1}, may be unsubstituted or independently substituted with one or more second substituent groups, referred to herein as R^{WW.2}; and each second substituent group, R^{WW.2}, may be unsubstituted or independently substituted with one or more third substituent groups, referred to herein as R^{WW.3}; and each third substituent group, R^{WW.3}, is unsubstituted. Each first substituent group is optionally different. Each second substituent group is optionally different. Each third substituent group is optionally different. In the context of two different R^{WW} substituents joined together to form an openly substituted ring, the “WW” symbol in the R^{WW.1}, R^{WW.2} and R^{WW.3} refers to the designated number of one of the two different R^{WW} substituents. For example, in embodiments where R^{100A} and R^{100B} are optionally joined together to form an openly substituted ring, R^{WW.1} is R^{100A.1}, R^{WW.2} is R^{100A.2}, and R^{WW.3} is R^{100A.3}. Alternatively, in embodiments where R^{100A} and R^{100B} are optionally joined together to form an openly substituted ring, R^{WW.1} is R^{100B.1}, R^{WW.2} is R^{100B.2}, and R^{WW.3} is R^{100B.3}. R^{WW.1}, R^{WW.2} and R^{WW.3} in this paragraph are as defined in the preceding paragraphs.

R^{LWW.1} is independently oxo, halogen, —CX^{LWW.1}₃, —CHX^{LWW.1}₂, —CH₂X^{LWW.1}, —OCX^{LWW.1}₃, —OCH₂X^{LWW.1}, —OCHX^{LWW.1}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, R^{LWW.2}-substituted or unsubstituted alkyl (e.g., C₁-C₈, C₁-C₆, C₁-C₄, or C₁-C₂), R^{LWW.2}-substituted or unsubstituted heteroalkyl (e.g., 2 to 8 membered, 2 to 6 membered, 4 to 6 membered, 2 to 3 membered, or 4 to 5 membered), R^{LWW.2}-substituted or unsubstituted cycloalkyl (e.g., C₃-C₈, C₃-C₆, C₄-C₆, or C₅-C₆), R^{LWW.2}-substituted or unsubstituted heterocycloalkyl (e.g., 3 to 8 membered, 3 to 6 membered, 4 to 6 membered, 4 to 5 membered, or 5 to 6 membered), R^{LWW.2}-substituted or unsubstituted aryl (e.g., C₆-C₁₂, C₆-C₁₀, or phenyl), or R^{LWW.2}-substituted or unsubstituted heteroaryl (e.g., 5 to 12 membered, 5 to 10 membered, 5 to 9 membered, or 5 to 6 membered). In embodiments, R^{LWW.1} is independently oxo, halogen, —CX^{LWW.1}₃, —CHX^{LWW.1}₂, —CH₂X^{LWW.1}, OCX^{LWW.1}₃, —OCH₂X^{LWW.1}, OCHX^{LWW.1}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{LWW.1} is independently —F, —Cl, —Br, or —I.

R^{LWW.2} is independently oxo, halogen, —CX^{LWW.2}₃, —CHX^{LWW.2}₂, —CH₂X^{LWW.2}, —OCX^{LWW.2}₃, —OCH₂X^{LWW.2}, —OCHX^{LWW.2}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, R^{LWW.3}-substituted or unsubstituted alkyl (e.g., C₁-C₈, C₁-C₆, C₁-C₄, or C₁-C₂), R^{LWW.3}-substituted or unsubstituted heteroalkyl (e.g., 2 to 8 membered, 2 to 6 membered, 4 to 6 membered, 2 to 3 membered, or 4 to 5 membered), R^{WW.3}-substituted or unsubstituted cycloalkyl (e.g., C₃-C₈, C₃-C₆, C₄-C₆, or C₅-C₆), R^{LWW.3}-substituted or unsubstituted heterocycloalkyl (e.g., 3 to 8 membered, 3 to 6 membered, 4 to 6 membered, 4 to 5 membered, or 5 to 6 membered), R^{LWW.3}-substituted or unsubstituted aryl (e.g., C₆-C₁₂, C₆-C₁₀, or phenyl), or R^{LWW.3}-substituted or unsubstituted heteroaryl (e.g., 5 to 12 membered, 5 to 10 membered, 5 to 9 membered, or 5 to 6 membered). In embodiments, R^{LWW.2} is independently oxo, halogen, —CX^{LWW.2}₃, —CHX^{LWW.2}₂, —CH₂X^{LWW.2}, —OCX^{LWW.2}₃, —OCH₂X^{LWW.2}, —OCHX^{LWW.2}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{LWW.2} is independently —F, —Cl, —Br, or —I.

R^{LWW.3} is independently oxo, halogen, —CX^{LWW.3}₃, —CHX^{LWW.3}₂, —CH₂X^{LWW.3}, —OCX^{LWW.3}₃, —OCH₂X^{LWW.3}, —OCHX^{LWW.3}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{LWW.3} is independently —F, —Cl, —Br, or —I.

In the event that any R group recited in a claim or chemical formula description set forth herein (R^{WW} substituent) is not specifically defined in this disclosure, then that R group (R^{WW} group) is hereby defined as independently oxo, halogen, —CX^{WW}₃, —CHX^{WW}₂, —CH₂X^{WW}, —OCX^{WW}₃, —OCH₂X^{WW}, —OCHX^{WW}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, R^{WW.1}-substituted or unsubstituted alkyl (e.g., C₁-C₈, C₁-C₆, C₁-C₄, or C₁-C₂), R^{WW.1}-substituted or unsubstituted heteroalkyl (e.g., 2 to 8 membered, 2 to 6 membered, 4 to 6 membered, 2 to 3 membered, or 4 to 5 membered), R^{WW.1}-substituted or unsubstituted cycloalkyl (e.g., C₃-C₈, C₃-C₆, C₄-C₆, or C₅-C₆), R^{WW.1}-substituted or unsubstituted heterocycloalkyl (e.g., 3 to 8 membered, 3 to 6 membered, 4 to 6 membered, 4 to 5 membered, or 5 to 6 membered), R^{WW.1}-substituted or unsubstituted aryl (e.g., C₆-C₁₂, C₆-C₁₀, or phenyl), or R^{WW.1}-substituted or unsubstituted heteroaryl (e.g., 5 to 12 membered, 5 to 10 membered, 5 to 9 membered, or 5 to 6 membered). X^{WW} is independently —F, —Cl, —Br, or —I. Again, “WW” represents the stated superscript number of the subject R group (e.g., 1, 2, 3, 1A, 2A, 3A, 1B, 2B, 3B, etc.). R^{WW.1}, R^{WW.2}, and R^{WW.3} are as defined above.

In the event that any L linker group recited in a claim or chemical formula description set forth herein (i.e., an L^{WW} substituent) is not explicitly defined, then that L group (L^{WW} group) is herein defined as independently a bond, —O—, —NH—, —C(O)—, —C(O)NH—, —NHC(O)—, —NHC(O)NH—, —NHC(NH)NH—, —C(O)O—, —OC(O)—, —S—, —SO₂—, —SO₂NH—, R^{LWW.1}-substituted or unsubstituted alkylene (e.g., C₁-C₈, C₁-C₆, C₁-C₄, or C₁-C₂), R^{LWW.1} substituted or unsubstituted heteroalkylene (e.g., 2 to 8 membered, 2 to 6 membered, 4 to 6 membered, 2 to 3 membered, or 4 to 5 membered), R^{LWW.1}-substituted or unsubstituted cycloalkylene (e.g., C₃-C₈, C₃-C₆, C₄-C₆, or C₅-C₆), R^{LWW.1}-substituted or unsubstituted heterocycloalkylene (e.g., 3 to 8 membered, 3 to 6 membered, 4 to 6 membered, 4 to 5 membered, or 5 to 6 membered), R^{LWW.1}-substituted or unsubstituted arylene (e.g., C₆-C₁₂, C₆-C₁₀, or phenyl), or R^{LWW.1}-substituted or unsubstituted heteroarylene (e.g., 5 to 12 membered, 5 to 10 membered, 5 to 9 membered, or 5 to 6 membered). Again, “WW” represents the stated superscript number of the subject L group (1, 2, 3, 1A, 2A, 3A, 1B, 2B, 3B, etc.). R^{LWW.1}, as well as R^{LWW.2} and R^{LWW.3} are as defined above.

Certain compounds of the present disclosure possess asymmetric carbon atoms (optical or chiral centers) or double bonds; the enantiomers, racemates, diastereomers, tautomers, geometric isomers, stereoisomeric forms that may be defined, in terms of absolute stereochemistry, as (R)- or (S)- or, as (D)- or (L)- for amino acids, and individual isomers are encompassed within the scope of the present disclosure. The compounds of the present disclosure do not include those that are known in art to be too unstable to synthesize and/or isolate. The present disclosure is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this disclosure may exist in tautomeric forms, all such tautomeric forms of the compounds being within the scope of the disclosure.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this disclosure.

The compounds of the present disclosure may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present disclosure, whether radioactive or not, are encompassed within the scope of the present disclosure.

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

As used herein, the terms “bioconjugate” and “bioconjugate linker” refer to the resulting association between atoms or molecules of bioconjugate reactive groups or bioconjugate reactive moieties. The association can be direct or indirect. For example, a conjugate between a first bioconjugate reactive group (e.g., —NH₂, —COOH, —N-hydroxysuccinimide, or -maleimide) and a second bioconjugate reactive group (e.g., sulfhydryl, sulfur-containing amino acid, amine, amine sidechain containing amino acid, or carboxylate) provided herein can be direct, e.g., by covalent bond or linker (e.g., a first linker of second linker), or indirect, e.g., by non-covalent bond (e.g., electrostatic interactions (e.g., ionic bond, hydrogen bond, halogen bond), van der Waals interactions (e.g., dipole-dipole, dipole-induced dipole, London dispersion), ring stacking (pi effects), hydrophobic interactions and the like). In embodiments, bioconjugates or bioconjugate linkers are formed using bioconjugate chemistry (i.e., the association of two bioconjugate reactive groups) including, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982. In embodiments, the first bioconjugate reactive group (e.g., maleimide moiety) is covalently attached to the second bioconjugate reactive group (e.g., a sulfhydryl). In embodiments, the first bioconjugate reactive group (e.g., haloacetyl moiety) is covalently attached to the second bioconjugate reactive group (e.g., a sulfhydryl). In embodiments, the first bioconjugate reactive group (e.g., pyridyl moiety) is covalently attached to the second bioconjugate reactive group (e.g., a sulfhydryl). In embodiments, the first bioconjugate reactive group (e.g., —N-hydroxysuccinimide moiety) is covalently attached to the second bioconjugate reactive group (e.g., an amine). In embodiments, the first bioconjugate reactive group (e.g., maleimide moiety) is covalently attached to the second bioconjugate reactive group (e.g., a sulfhydryl). In embodiments, the first bioconjugate reactive group (e.g., -sulfo-N-hydroxysuccinimide moiety) is covalently attached to the second bioconjugate reactive group (e.g., an amine).

Useful bioconjugate reactive moieties used for bioconjugate chemistries herein include, for example: (a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters; (b) hydroxyl groups which can be converted to esters, ethers, aldehydes, etc.; (c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom; (d) dienophile groups which are capable of participating in Diels-Alder reactions such as, for example, maleimido or maleimide groups; (e) aldehyde or ketone groups such that subsequent derivatization is possible via formation of carbonyl derivatives such as, for example, imines, hydrazones, semicarbazones or oximes, or via such mechanisms as Grignard addition or alkyllithium addition; (f) sulfonyl halide groups for subsequent reaction with amines, for example, to form sulfonamides; (g) thiol groups, which can be converted to disulfides, reacted with acyl halides, or bonded to metals such as gold, or react with maleimides; (h) amine or sulfhydryl groups (e.g., present in cysteine), which can be, for example, acylated, alkylated or oxidized; (i) alkenes, which can undergo, for example, cycloadditions, acylation, Michael addition, etc.; (j) epoxides, which can react with, for example, amines and hydroxyl compounds; (k) phosphoramidites and other standard functional groups useful in nucleic acid synthesis; (l) metal silicon oxide bonding; (m) metal bonding to reactive phosphorus groups (e.g., phosphines) to form, for example, phosphate diester bonds; (n) azides coupled to alkynes using copper catalyzed cycloaddition click chemistry; and (o) biotin conjugate can react with avidin or streptavidin to form an avidin-biotin complex or streptavidin-biotin complex.

The bioconjugate reactive groups can be chosen such that they do not participate in, or interfere with, the chemical stability of the conjugate described herein. Alternatively, a reactive functional group can be protected from participating in the crosslinking reaction by the presence of a protecting group. In embodiments, the bioconjugate comprises a molecular entity derived from the reaction of an unsaturated bond, such as a maleimide, and a sulfhydryl group.

“Analog,” “analogue,” or “derivative” is used in accordance with its plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure or origin to a reference compound.

The terms “a” or “an”, as used in herein means one or more. In addition, the phrase “substituted with a[n]”, as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl”, the group may contain one or more unsubstituted C₁-C₂₀ alkyls, and/or one or more unsubstituted 2 to 20 membered heteroalkyls.

Moreover, where a moiety is substituted with an R substituent, the group may be referred to as “R-substituted.” Where a moiety is R-substituted, the moiety is substituted with at least one R substituent and each R substituent is optionally different. Where a particular R group is present in the description of a chemical genus (such as Formula (I)), a Roman alphabetic symbol may be used to distinguish each appearance of that particular R group. For example, where multiple R¹³ substituents are present, each R¹³ substituent may be distinguished as R^{13.A}, R^{13.B}, R^{13.C}, R^{13.D}, etc., wherein each of R^{13.A}, R^{13.B}, R^{13.C}, R^{13.D}, etc. is defined within the scope of the definition of R¹³ and optionally differently.

Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

The term “pharmaceutically acceptable salts” is meant to include salts of the active compounds that are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

Thus, the compounds of the present disclosure may exist as salts, such as with pharmaceutically acceptable acids. The present disclosure includes such salts. Non-limiting examples of such salts include hydrochlorides, hydrobromides, phosphates, sulfates, methanesulfonates, nitrates, maleates, acetates, citrates, fumarates, proprionates, tartrates (e.g., (+)-tartrates, (−)-tartrates, or mixtures thereof including racemic mixtures), succinates, benzoates, and salts with amino acids such as glutamic acid, and quaternary ammonium salts (e.g., methyl iodide, ethyl iodide, and the like). These salts may be prepared by methods known to those skilled in the art.

The neutral forms of the compounds are preferably regenerated by contacting the salt with a base or acid and isolating the parent compound in the conventional manner. The parent form of the compound may differ from the various salt forms in certain physical properties, such as solubility in polar solvents.

In addition to salt forms, the present disclosure provides compounds, which are in a prodrug form. Prodrugs of the compounds described herein are those compounds that readily undergo chemical changes under physiological conditions to provide the compounds of the present disclosure. Prodrugs of the compounds described herein may be converted in vivo after administration. Additionally, prodrugs can be converted to the compounds of the present disclosure by chemical or biochemical methods in an ex vivo environment, such as, for example, when contacted with a suitable enzyme or chemical reagent.

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

A polypeptide, or a cell is “recombinant” when it is artificial or engineered, or derived from or contains an artificial or engineered protein or nucleic acid (e.g., non-natural or not wild type). For example, a polynucleotide that is inserted into a vector or any other heterologous location, e.g., in a genome of a recombinant organism, such that it is not associated with nucleotide sequences that normally flank the polynucleotide as it is found in nature is a recombinant polynucleotide. A protein expressed in vitro or in vivo from a recombinant polynucleotide is an example of a recombinant polypeptide. Likewise, a polynucleotide sequence that does not appear in nature, for example a variant of a naturally occurring gene, is recombinant.

“Co-administer” is meant that a composition described herein is administered at the same time, just prior to, or just after the administration of one or more additional therapies. The compounds of the invention can be administered alone or can be co-administered to the patient. Co-administration is meant to include simultaneous or sequential administration of the compounds individually or in combination (more than one compound). Thus, the preparations can also be combined, when desired, with other active substances (e.g., to reduce metabolic degradation).

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaroytic cells. Prokaryotic cells include but are not limited to bacteria. Eukaryotic cells include but are not limited to yeast cells and cells derived from plants and animals, for example mammalian, insect (e.g., spodoptera) and human cells. Cells may be useful when they are naturally nonadherent or have been treated not to adhere to surfaces, for example by trypsinization.

The terms “treating” or “treatment” refers to any indicia of success in the treatment or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. For example, the certain methods presented herein successfully treat cancer by decreasing the incidence of cancer and or causing remission of cancer. In some embodiments of the compositions or methods described herein, treating cancer includes slowing the rate of growth or spread of cancer cells, reducing metastasis, or reducing the growth of metastatic tumors. The term “treating” and conjugations thereof, include prevention of an injury, pathology, condition, or disease. In embodiments, treating is preventing. In embodiments, treating does not include preventing. In embodiments, the treating or treatment is no prophylactic treatment.

An “effective amount” is an amount sufficient for a compound to accomplish a stated purpose relative to the absence of the compound (e.g., achieve the effect for which it is administered, treat a disease, reduce enzyme activity, increase enzyme activity, reduce signaling pathway, reduce one or more symptoms of a disease or condition. An example of an “effective amount” is an amount sufficient to contribute to the treatment, prevention, or reduction of a symptom or symptoms of a disease, which could also be referred to as a “therapeutically effective amount” when referred to in this context. A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. An “activity increasing amount,” as used herein, refers to an amount of agonist required to increase the activity of an enzyme relative to the absence of the agonist. A “function increasing amount,” as used herein, refers to the amount of agonist required to increase the function of an enzyme or protein relative to the absence of the agonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is the measurement of the activity (e.g., signaling pathway) of a protein in the absence of a compound as described herein (including embodiments, examples, figures, or Tables).

“Contacting” is used in accordance with its plain ordinary meaning and refers to the process of allowing at least two distinct species (e.g., chemical compounds including biomolecules, or cells) to become sufficiently proximal to react, interact or physically touch. It should be appreciated; however, the resulting reaction product can be produced directly from a reaction between the added reagents or from an intermediate from one or more of the added reagents which can be produced in the reaction mixture.

The term “contacting” may include allowing two species to react, interact, or physically touch, wherein the two species may be a compound as described herein and a cellular component (e.g., protein, ion, lipid, nucleic acid, nucleotide, amino acid, protein, particle, organelle, cellular compartment, microorganism, virus, lipid droplet, vesicle, small molecule, protein complex, protein aggregate, or macromolecule). In some embodiments contacting includes allowing a compound described herein to interact with a cellular component (e.g., protein, ion, lipid, nucleic acid, nucleotide, amino acid, protein, particle, virus, lipid droplet, organelle, cellular compartment, microorganism, vesicle, small molecule, protein complex, protein aggregate, or macromolecule) that is involved in a signaling pathway.

As defined herein, the term “activation,” “activate,” “activating” and the like in reference to a protein refers to conversion of a protein into a biologically active derivative from an initial inactive or deactivated state. The terms reference activation, or activating, sensitizing, or up-regulating signal transduction or enzymatic activity or the amount of a protein decreased in a disease.

The terms “agonist,” “activator,” “upregulator,” etc. refer to a substance capable of detectably increasing the expression or activity of a given gene or protein. The agonist can increase expression or activity by at least 10%, 20%, 30%, 40%, 50%, 60%, 70%, 80%, 90%, 95%, 96%, 97%, 98%, or 99% in comparison to a control in the absence of the agonist. In certain instances, expression or activity is 1.5-fold, 2-fold, 3-fold, 4-fold, 5-fold, 10-fold or higher than the expression or activity in the absence of the agonist.

As defined herein, the term “inhibition,” “inhibit,” “inhibiting” and the like in reference to a cellular component-inhibitor interaction means negatively affecting (e.g., decreasing) the activity or function of the cellular component (e.g., decreasing the signaling pathway stimulated by a cellular component (e.g., protein, ion, lipid, virus, lipid droplet, nucleic acid, nucleotide, amino acid, protein, particle, organelle, cellular compartment, microorganism, vesicle, small molecule, protein complex, protein aggregate, or macromolecule)), relative to the activity or function of the cellular component in the absence of the inhibitor. In embodiments, inhibition means negatively affecting (e.g., decreasing) the concentration or levels of the cellular component relative to the concentration or level of the cellular component in the absence of the inhibitor. In some embodiments, inhibition refers to reduction of a disease or symptoms of disease. In some embodiments, inhibition refers to a reduction in the activity of a signal transduction pathway or signaling pathway (e.g., reduction of a pathway involving the cellular component). Thus, inhibition includes, at least in part, partially or totally blocking stimulation, decreasing, preventing, or delaying activation, or inactivating, desensitizing, or down-regulating the signaling pathway or enzymatic activity or the amount of a cellular component.

The terms “inhibitor,” “repressor,” “antagonist,” or “downregulator” interchangeably refer to a substance capable of detectably decreasing the expression or activity of a given gene or protein. The antagonist can decrease expression or activity by at least 10%, 20%, 30%, 40%, 50%, 60%, 70%, 80%, 90%, 95%, 96%, 97%, 98%, or 99% in comparison to a control in the absence of the antagonist. In certain instances, expression or activity is 1.5-fold, 2-fold, 3-fold, 4-fold, 5-fold, 10-fold or lower than the expression or activity in the absence of the antagonist.

The term “modulator” refers to a composition that increases or decreases the level of a target molecule or the function of a target molecule or the physical state of the target of the molecule (e.g., a target may be a cellular component (e.g., protein, ion, lipid, virus, lipid droplet, nucleic acid, nucleotide, amino acid, protein, particle, organelle, cellular compartment, microorganism, vesicle, small molecule, protein complex, protein aggregate, or macromolecule)) relative to the absence of the composition.

The term “expression” includes any step involved in the production of the polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

The term “modulate” is used in accordance with its plain ordinary meaning and refers to the act of changing or varying one or more properties. “Modulation” refers to the process of changing or varying one or more properties. For example, as applied to the effects of a modulator on a target protein, to modulate means to change by increasing or decreasing a property or function of the target molecule or the amount of the target molecule.

“Patient”, “patient in need thereof”, “subject”, or “subject in need thereof” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In some embodiments, a patient is human. In embodiments, a patient in need thereof is human. In embodiments, a subject is human. In embodiments, a subject in need thereof is human.

“Disease” or “condition” refer to a state of being or health status of a patient or subject capable of being treated with the compounds or methods provided herein. In some embodiments, the disease is a disease related to (e.g., caused by) a cellular component (e.g., protein, ion, lipid, nucleic acid, nucleotide, amino acid, protein, particle, organelle, cellular compartment, microorganism, vesicle, small molecule, protein complex, protein aggregate, or macromolecule). In embodiments, the disease is cancer (e.g., rectal carcinoma, colorectal adenocarcinoma, colorectal carcinoma, non-small cell lung carcinoma, squamous cell lung carcinoma, myelodysplastic syndrome, or acute myeloid leukemia). In embodiments, the disease is Costello syndrome.

As used herein, the term “cancer” refers to all types of cancer, neoplasm or malignant tumors found in mammals (e.g., humans), including leukemia, lymphoma, carcinomas and sarcomas. Exemplary cancers that may be treated with a compound or method provided herein include cancer of the thyroid, endocrine system, brain, breast, cervix, colon, head and neck, liver, kidney, lung, non-small cell lung, melanoma, mesothelioma, ovary, sarcoma, stomach, uterus, medulloblastoma, colorectal cancer, or pancreatic cancer. Additional examples include, Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound or method provided herein include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia.

As used herein, the term “lymphoma” refers to a group of cancers affecting hematopoietic and lymphoid tissues. It begins in lymphocytes, the blood cells that are found primarily in lymph nodes, spleen, thymus, and bone marrow. Two main types of lymphoma are non-Hodgkin lymphoma and Hodgkin's disease. Hodgkin's disease represents approximately 15% of all diagnosed lymphomas. This is a cancer associated with Reed-Sternberg malignant B lymphocytes. Non-Hodgkin's lymphomas (NHL) can be classified based on the rate at which cancer grows and the type of cells involved. There are aggressive (high grade) and indolent (low grade) types of NHL. Based on the type of cells involved, there are B-cell and T-cell NHLs. Exemplary B-cell lymphomas that may be treated with a compound or method provided herein include, but are not limited to, small lymphocytic lymphoma, Mantle cell lymphoma, follicular lymphoma, marginal zone lymphoma, extranodal (MALT) lymphoma, nodal (monocytoid B-cell) lymphoma, splenic lymphoma, diffuse large cell B-lymphoma, Burkitt's lymphoma, lymphoblastic lymphoma, immunoblastic large cell lymphoma, or precursor B-lymphoblastic lymphoma. Exemplary T-cell lymphomas that may be treated with a compound or method provided herein include, but are not limited to, cutaneous T-cell lymphoma, peripheral T-cell lymphoma, anaplastic large cell lymphoma, mycosis fungoides, and precursor T-lymphoblastic lymphoma.

The term “sarcoma” generally refers to a tumor which is made up of a substance like the embryonic connective tissue and is generally composed of closely packed cells embedded in a fibrillar or homogeneous substance. Sarcomas that may be treated with a compound or method provided herein include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

The term “melanoma” is taken to mean a tumor arising from the melanocytic system of the skin and other organs. Melanomas that may be treated with a compound or method provided herein include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, or superficial spreading melanoma.

The term “carcinoma” refers to a malignant new growth made up of epithelial cells tending to infiltrate the surrounding tissues and give rise to metastases. Exemplary carcinomas that may be treated with a compound or method provided herein include, for example, medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, or carcinoma villosum.

As used herein, the terms “metastasis,” “metastatic,” and “metastatic cancer” can be used interchangeably and refer to the spread of a proliferative disease or disorder, e.g., cancer, from one organ or another non-adjacent organ or body part. “Metastatic cancer” is also called “Stage IV cancer.” Cancer occurs at an originating site, e.g., breast, which site is referred to as a primary tumor, e.g., primary breast cancer. Some cancer cells in the primary tumor or originating site acquire the ability to penetrate and infiltrate surrounding normal tissue in the local area and/or the ability to penetrate the walls of the lymphatic system or vascular system circulating through the system to other sites and tissues in the body. A second clinically detectable tumor formed from cancer cells of a primary tumor is referred to as a metastatic or secondary tumor. When cancer cells metastasize, the metastatic tumor and its cells are presumed to be similar to those of the original tumor. Thus, if lung cancer metastasizes to the breast, the secondary tumor at the site of the breast consists of abnormal lung cells and not abnormal breast cells. The secondary tumor in the breast is referred to a metastatic lung cancer. Thus, the phrase metastatic cancer refers to a disease in which a subject has or had a primary tumor and has one or more secondary tumors. The phrases non-metastatic cancer or subjects with cancer that is not metastatic refers to diseases in which subjects have a primary tumor but not one or more secondary tumors. For example, metastatic lung cancer refers to a disease in a subject with or with a history of a primary lung tumor and with one or more secondary tumors at a second location or multiple locations, e.g., in the breast.

The terms “cutaneous metastasis” and “skin metastasis” refer to secondary malignant cell growths in the skin, wherein the malignant cells originate from a primary cancer site (e.g., breast). In cutaneous metastasis, cancerous cells from a primary cancer site may migrate to the skin where they divide and cause lesions. Cutaneous metastasis may result from the migration of cancer cells from breast cancer tumors to the skin.

The term “visceral metastasis” refers to secondary malignant cell growths in the internal organs (e.g., heart, lungs, liver, pancreas, intestines) or body cavities (e.g., pleura, peritoneum), wherein the malignant cells originate from a primary cancer site (e.g., head and neck, liver, breast). In visceral metastasis, cancerous cells from a primary cancer site may migrate to the internal organs where they divide and cause lesions. Visceral metastasis may result from the migration of cancer cells from liver cancer tumors or head and neck tumors to internal organs.

As used herein, the term “RASopathy” refers to a disease caused by germline mutations of genes encoding components of the RAS/MAPK signaling pathway. In embodiments, the RASopathy is a mosaic RASopathy. In embodiments, the RASopathy is a germline RASopathy. In embodiments, the RASopathy is a developmental syndrome. In embodiments, the RASopathy is Noonan syndrome. In embodiments, the RASopathy is epidermal nevus. In embodiments, the RASopathy is Schimmelpenning syndrome. In embodiments, the RASopathy is sebaceous nevus. In embodiments, the RASopathy is Talipes equinovarus (e.g., congenital Talipes equinovarus). In embodiments, the RASopathy is PIK3CA-related overgrowth syndrome (PROS). In embodiments, the RASopathy is PTEN-Hamartoma of the soft tissue (PHOST). In embodiments, the RASopathy is fibroadipose overgrowth, hemihyperplasia-multiple lipomatosis, congenital lipomatous overgrowth, vascular malformations, epidermal nevus, spinal and skeletal syndrome, macrodactyly syndrome, megalocephaly syndrome, or congenital diffuse infiltrative lipomatosis. In embodiments, the RASopathy is Klippel-Trenaunay syndrome (KTS), venous malformation, or lymphatic malformation. In embodiments, the RASopathy is capillary malformation-AV malformation syndrome. In embodiments, the RASopathy is autoimmune lymphoproliferative syndrome. In embodiments, the RASopathy is cardiofaciocutaneous syndrome. In embodiments, the RASopathy is hereditary gingival fibromatosis type 1. In embodiments, the RASopathy is neurofibromatosis type 1. In embodiments, the RASopathy is Costello syndrome. In embodiments, the RASopathy is Legius syndrome.

As used herein, the term “Switch II GTPase protein-associated disease” refers to any disease or condition caused by aberrant activity or signaling of a Switch II GTPase protein. In embodiments, the Switch II GTPase protein-associated disease is cancer (e.g., rectal carcinoma, colorectal adenocarcinoma, colorectal carcinoma, non-small cell lung carcinoma, squamous cell lung carcinoma, myelodysplastic syndrome, or acute myeloid leukemia). In embodiments, the Switch II GTPase protein-associated disease is a RASopathy.

As used herein, the term “K-Ras(G12S)-associated disease” refers to any disease or condition caused by aberrant activity or signaling of K-Ras(G12S). In embodiments, the K-Ras(G12S)-associated disease is cancer (e.g., rectal carcinoma, colorectal adenocarcinoma, colorectal carcinoma, non-small cell lung carcinoma, squamous cell lung carcinoma, myelodysplastic syndrome, or acute myeloid leukemia). In embodiments, the K-Ras(G12S)-associated disease is a RASopathy.

As used herein, the term “H-Ras(G12S)-associated disease” refers to any disease or condition caused by aberrant activity or signaling of H-Ras(G12S). In embodiments, the H-Ras(G12S)-associated disease is cancer (e.g., rectal carcinoma, colorectal adenocarcinoma, colorectal carcinoma, non-small cell lung carcinoma, squamous cell lung carcinoma, myelodysplastic syndrome, or acute myeloid leukemia). In embodiments, the H-Ras(G12S)-associated disease is Costello syndrome. In embodiments, the H-Ras(G12S)-associated disease is a RASopathy.

As used herein, the term “N-Ras(G12S)-associated disease” refers to any disease or condition caused by aberrant activity or signaling of N-Ras(G12S). In embodiments, the N-Ras(G12S)-associated disease is cancer (e.g., rectal carcinoma, colorectal adenocarcinoma, colorectal carcinoma, non-small cell lung carcinoma, squamous cell lung carcinoma, myelodysplastic syndrome, or acute myeloid leukemia). In embodiments, the N-Ras(G12S)-associated disease is a RASopathy.

As used herein, the term “K-Ras(G13S)-associated disease” refers to any disease or condition caused by aberrant activity or signaling of K-Ras(G13S). In embodiments, the K-Ras(G13S)-associated disease is cancer (e.g., rectal carcinoma, colorectal adenocarcinoma, colorectal carcinoma, non-small cell lung carcinoma, squamous cell lung carcinoma, myelodysplastic syndrome, or acute myeloid leukemia). In embodiments, the K-Ras(G13S)-associated disease is a RASopathy.

As used herein, the term “H-Ras(G13S)-associated disease” refers to any disease or condition caused by aberrant activity or signaling of H-Ras(G13S). In embodiments, the H-Ras(G13S)-associated disease is cancer (e.g., rectal carcinoma, colorectal adenocarcinoma, colorectal carcinoma, non-small cell lung carcinoma, squamous cell lung carcinoma, myelodysplastic syndrome, or acute myeloid leukemia). In embodiments, the H-Ras(G13S)-associated disease is a RASopathy.

As used herein, the term “N-Ras(G13S)-associated disease” refers to any disease or condition caused by aberrant activity or signaling of N-Ras(G13S). In embodiments, the N-Ras(G13S)-associated disease is cancer (e.g., rectal carcinoma, colorectal adenocarcinoma, colorectal carcinoma, non-small cell lung carcinoma, squamous cell lung carcinoma, myelodysplastic syndrome, or acute myeloid leukemia). In embodiments, the N-Ras(G13S)-associated disease is a RASopathy.

As used herein, the term “K-Ras(G12T)-associated disease” refers to any disease or condition caused by aberrant activity or signaling of K-Ras(G12T). In embodiments, the K-Ras(G12T)-associated disease is cancer (e.g., rectal carcinoma, colorectal adenocarcinoma, colorectal carcinoma, non-small cell lung carcinoma, squamous cell lung carcinoma, myelodysplastic syndrome, or acute myeloid leukemia). In embodiments, the K-Ras(G12T)-associated disease is a RASopathy.

As used herein, the term “H-Ras(G12T)-associated disease” refers to any disease or condition caused by aberrant activity or signaling of H-Ras(G12T). In embodiments, the H-Ras(G12T)-associated disease is cancer (e.g., rectal carcinoma, colorectal adenocarcinoma, colorectal carcinoma, non-small cell lung carcinoma, squamous cell lung carcinoma, myelodysplastic syndrome, or acute myeloid leukemia). In embodiments, the H-Ras(G12T)-associated disease is a RASopathy.

As used herein, the term “N-Ras(G12T)-associated disease” refers to any disease or condition caused by aberrant activity or signaling of N-Ras(G12T). In embodiments, the N-Ras(G12T)-associated disease is cancer (e.g., rectal carcinoma, colorectal adenocarcinoma, colorectal carcinoma, non-small cell lung carcinoma, squamous cell lung carcinoma, myelodysplastic syndrome, or acute myeloid leukemia). In embodiments, the N-Ras(G12T)-associated disease is a RASopathy.

As used herein, the term “K-Ras(G12D)-associated disease” refers to any disease or condition caused by aberrant activity or signaling of K-Ras(G12D). In embodiments, the K-Ras(G12D)-associated disease is cancer. In embodiments, the K-Ras(G12D)-associated disease is a RASopathy.

As used herein, the term “H-Ras(G12D)-associated disease” refers to any disease or condition caused by aberrant activity or signaling of H-Ras(G12D). In embodiments, the H-Ras(G12D)-associated disease is cancer. In embodiments, the H-Ras(G12D)-associated disease is a RASopathy.

As used herein, the term “N-Ras(G12D)-associated disease” refers to any disease or condition caused by aberrant activity or signaling of N-Ras(G12D). In embodiments, the N-Ras(G12D)-associated disease is cancer. In embodiments, the N-Ras(G12D)-associated disease is a RASopathy.

As used herein, the term “K-Ras(G13D)-associated disease” refers to any disease or condition caused by aberrant activity or signaling of K-Ras(G13D). In embodiments, the K-Ras(G13D)-associated disease is cancer. In embodiments, the K-Ras(G13D)-associated disease is a RASopathy.

As used herein, the term “H-Ras(G13D)-associated disease” refers to any disease or condition caused by aberrant activity or signaling of H-Ras(G13D). In embodiments, the H-Ras(G13D)-associated disease is cancer. In embodiments, the H-Ras(G13D)-associated disease is a RASopathy.

As used herein, the term “N-Ras(G13D)-associated disease” refers to any disease or condition caused by aberrant activity or signaling of N-Ras(G13D). In embodiments, the N-Ras(G13D)-associated disease is cancer. In embodiments, the N-Ras(G13D)-associated disease is a RASopathy.

As used herein, the term “K-Ras(G12E)-associated disease” refers to any disease or condition caused by aberrant activity or signaling of K-Ras(G12E). In embodiments, the K-Ras(G12E)-associated disease is cancer. In embodiments, the K-Ras(G12E)-associated disease is a RASopathy.

As used herein, the term “H-Ras(G12E)-associated disease” refers to any disease or condition caused by aberrant activity or signaling of H-Ras(G12E). In embodiments, the H-Ras(G12E)-associated disease is cancer. In embodiments, the H-Ras(G12E)-associated disease is a RASopathy.

As used herein, the term “N-Ras(G12E)-associated disease” refers to any disease or condition caused by aberrant activity or signaling of N-Ras(G12E). In embodiments, the N-Ras(G12E)-associated disease is cancer. In embodiments, the N-Ras(G12E)-associated disease is a RASopathy.

The term “drug” is used in accordance with its common meaning and refers to a substance which has a physiological effect (e.g., beneficial effect, is useful for treating a subject) when introduced into or to a subject (e.g., in or on the body of a subject or patient). A drug moiety is a radical of a drug.

A “detectable agent,” “detectable compound,” “detectable label,” or “detectable moiety” is a substance (e.g., element), molecule, or composition detectable by spectroscopic, photochemical, biochemical, immunochemical, chemical, magnetic resonance imaging, or other physical means. For example, detectable agents include ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y, ⁹⁰Y, ⁸⁹Sr, ⁸⁹Z, ⁹⁴Tc, ⁹⁴Tc, ^{99m}Tc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴I, ¹⁹⁸Au, ¹⁹⁹Au, ²¹¹At, ²¹¹Pb, ²¹²Bi, ²¹²Pb, ²¹³Bi, ²²³Ra, ²²⁵Ac, Cr, V, Mn, Fe, Co, Ni, Cu, La, Ce, Pr, Nd, Pm, Sm, Eu, Gd, Tb, Dy, Ho, Er, Tm, Yb, Lu, ³²P, fluorophore (e.g., fluorescent dyes), modified oligonucleotides (e.g., moieties described in PCT/US2015/022063, which is incorporated herein by reference), electron-dense reagents, enzymes (e.g., as commonly used in an ELISA), biotin, digoxigenin, paramagnetic molecules, paramagnetic nanoparticles, ultrasmall superparamagnetic iron oxide (“USPIO”) nanoparticles, USPIO nanoparticle aggregates, superparamagnetic iron oxide (“SPIO”) nanoparticles, SPIO nanoparticle aggregates, monochrystalline iron oxide nanoparticles, monochrystalline iron oxide, nanoparticle contrast agents, liposomes or other delivery vehicles containing Gadolinium chelate (“Gd-chelate”) molecules, Gadolinium, radioisotopes, radionuclides (e.g., carbon-11, nitrogen-13, oxygen-15, fluorine-18, rubidium-82), fluorodeoxyglucose (e.g., fluorine-18 labeled), any gamma ray emitting radionuclides, positron-emitting radionuclide, radiolabeled glucose, radiolabeled water, radiolabeled ammonia, biocolloids, microbubbles (e.g., including microbubble shells including albumin, galactose, lipid, and/or polymers; microbubble gas core including air, heavy gas(es), perfluorcarbon, nitrogen, octafluoropropane, perflexane lipid microsphere, perflutren, etc.), iodinated contrast agents (e.g., iohexol, iodixanol, ioversol, iopamidol, ioxilan, iopromide, diatrizoate, metrizoate, ioxaglate), barium sulfate, thorium dioxide, gold, gold nanoparticles, gold nanoparticle aggregates, fluorophores, two-photon fluorophores, or haptens and proteins or other entities which can be made detectable, e.g., by incorporating a radiolabel into a peptide or antibody specifically reactive with a target peptide.

Radioactive substances (e.g., radioisotopes) that may be used as imaging and/or labeling agents in accordance with the embodiments of the disclosure include, but are not limited to, ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y, ⁹⁰Y, ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh, ¹¹¹Ag, ¹¹¹In, ¹²³I, ¹²⁴I, ¹²⁵I, ¹²⁵I, ¹⁴²Pr, ¹⁴³Pr, ¹⁴⁹Pm, ¹⁵³Sm, ¹⁵⁴⁻¹⁵⁸Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb ²¹²Bi, ²¹²Pb, ²¹³Bi, ²²³Ra and ²²⁵Ac. Paramagnetic ions that may be used as additional imaging agents in accordance with the embodiments of the disclosure include, but are not limited to, ions of transition and lanthanide metals (e.g., metals having atomic numbers of 21-29, 42, 43, 44, or 57-71). These metals include ions of Cr, V, Mn, Fe, Co, Ni, Cu, La, Ce, Pr, Nd, Pm, Sm, Eu, Gd, Tb, Dy, Ho, Er, Tm, Yb, and Lu.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

As used herein, the term “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about includes the specified value.

As used herein, the term “administering” is used in accordance with its plain and ordinary meaning and includes oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies, for example cancer therapies such as chemotherapy, hormonal therapy, radiotherapy, or immunotherapy. The compounds of the invention can be administered alone or can be co-administered to the patient. Co-administration is meant to include simultaneous or sequential administration of the compounds individually or in combination (more than one compound). Thus, the preparations can also be combined, when desired, with other active substances (e.g., to reduce metabolic degradation). The compositions of the present invention can be delivered by transdermally, by a topical route, formulated as applicator sticks, solutions, suspensions, emulsions, gels, creams, ointments, pastes, jellies, paints, powders, and aerosols.

The compounds described herein can be used in combination with one another, with other active agents known to be useful in treating a disease associated with cells expressing a disease associated cellular component, or with adjunctive agents that may not be effective alone, but may contribute to the efficacy of the active agent.

In some embodiments, co-administration includes administering one active agent within 0.5, 1, 2, 4, 6, 8, 10, 12, 16, 20, or 24 hours of a second active agent. Co-administration includes administering two active agents simultaneously, approximately simultaneously (e.g., within about 1, 5, 10, 15, 20, or 30 minutes of each other), or sequentially in any order. In some embodiments, co-administration can be accomplished by co-formulation, i.e., preparing a single pharmaceutical composition including both active agents. In other embodiments, the active agents can be formulated separately. In another embodiment, the active and/or adjunctive agents may be linked or conjugated to one another.

In therapeutic use for the treatment of a disease, compound utilized in the pharmaceutical compositions of the present invention may be administered at the initial dosage of about 0.001 mg/kg to about 1000 mg/kg daily. A daily dose range of about 0.01 mg/kg to about 500 mg/kg, or about 0.1 mg/kg to about 200 mg/kg, or about 1 mg/kg to about 100 mg/kg, or about 10 mg/kg to about 50 mg/kg, can be used. The dosages, however, may be varied depending upon the requirements of the patient, the severity of the condition being treated, and the compound or drug being employed. For example, dosages can be empirically determined considering the type and stage of disease (e.g., cancer, RASopathy, or Costello syndrome) diagnosed in a particular patient. The dose administered to a patient, in the context of the present invention, should be sufficient to affect a beneficial therapeutic response in the patient over time. The size of the dose will also be determined by the existence, nature, and extent of any adverse side effects that accompany the administration of a compound in a particular patient. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. For convenience, the total daily dosage may be divided and administered in portions during the day, if desired.

The term “associated” or “associated with” in the context of a substance or substance activity or function associated with a disease (e.g., a protein associated disease, disease associated with a cellular component) means that the disease (e.g., cancer, RASopathy, or Costello syndrome) is caused by (in whole or in part), or a symptom of the disease is caused by (in whole or in part) the substance or substance activity or function or the disease or a symptom of the disease may be treated by modulating (e.g., inhibiting or activating) the substance (e.g., cellular component). As used herein, what is described as being associated with a disease, if a causative agent, could be a target for treatment of the disease.

The term “aberrant” as used herein refers to different from normal. When used to describe enzymatic activity, aberrant refers to activity that is greater or less than a normal control or the average of normal non-diseased control samples. Aberrant activity may refer to an amount of activity that results in a disease, wherein returning the aberrant activity to a normal or non-disease-associated amount (e.g., by administering a compound or using a method as described herein), results in reduction of the disease or one or more disease symptoms.

The term “electrophilic” as used herein refers to a chemical group that is capable of accepting electron density. An “electrophilic substituent,” “electrophilic chemical moiety,” or “electrophilic moiety” refers to an electron-poor chemical group, substituent, or moiety (monovalent chemical group), which may react with an electron-donating group, such as a nucleophile, by accepting an electron pair or electron density to form a bond.

“Nucleophilic” as used herein refers to a chemical group that is capable of donating electron density.

The term “isolated,” when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide,” and “protein” are used interchangeably herein to refer to a polymer of amino acid residues, wherein the polymer may in embodiments be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence.

An amino acid residue in a protein “corresponds” to a given residue when it occupies the same essential structural position within the protein as the given residue. For example, a selected residue in a selected protein corresponds to G12 of K-Ras when the selected residue occupies the same essential spatial or other structural relationship as G12 of K-Ras. In some embodiments, where a selected protein is aligned for maximum homology with K-Ras, the position in the aligned selected protein aligning with G12 is said to correspond to G12. Instead of a primary sequence alignment, a three dimensional structural alignment can also be used, e.g., where the structure of the selected protein is aligned for maximum correspondence with K-Ras and the overall structures compared. In this case, an amino acid that occupies the same essential position as G12 in the structural model is said to correspond to the G12 residue.

The term “protein complex” is used in accordance with its plain ordinary meaning and refers to a protein which is associated with an additional substance (e.g., another protein, protein subunit, or a compound). Protein complexes typically have defined quaternary structure. The association between the protein and the additional substance may be a covalent bond. In embodiments, the association between the protein and the additional substance (e.g., compound) is via non-covalent interactions. In embodiments, a protein complex refers to a group of two or more polypeptide chains. Proteins in a protein complex are linked by non-covalent protein-protein interactions. A non-limiting example of a protein complex is the proteasome.

The term “protein aggregate” is used in accordance with its plain ordinary meaning and refers to an aberrant collection or accumulation of proteins (e.g., misfolded proteins). Protein aggregates are often associated with diseases (e.g., amyloidosis). Typically, when a protein misfolds as a result of a change in the amino acid sequence or a change in the native environment which disrupts normal non-covalent interactions, and the misfolded protein is not corrected or degraded, the unfolded/misfolded protein may aggregate. There are three main types of protein aggregates that may form: amorphous aggregates, oligomers, and amyloid fibrils. In embodiments, protein aggregates are termed aggresomes.

The term “Switch II” as used herein refers to a protein domain of a GTPase protein (e.g., Ras, K-Ras, H-Ras, or N-Ras) formed by residues corresponding to residues 60-76 of K-Ras, H-Ras, or N-Ras (e.g., K-Ras Switch II refers to residues 60-76 of K-Ras, H-Ras Switch II refers to residues 60-76 of H-Ras, N-Ras Switch II refers to residues 60-76 of N-Ras). A “Switch II Binding Pocket” is a cavity bound (the limits or boundaries of which are made), at least in part, by the amino acid residues that form Switch II. In some embodiments, a “Switch II Binding Pocket” is a cavity, in the GDP bound form of a GTPase protein (e.g., Ras, K-Ras, H-Ras, or N-Ras), bound (the limits or boundaries of which are made), at least in part, by the amino acid residues that form Switch II. A “Switch II Binding Pocket binding moiety” is a moiety of a compound (e.g., as described herein) that binds to the Switch II Binding Pocket.

The term “Switch II GTPase protein” as used herein refers to a GTPase protein including a Switch II. In embodiments, the Switch II GTPase protein includes a Switch II Binding Pocket. In embodiments, the Switch II GTPase protein is a Ras protein. In embodiments, the Switch II GTPase protein is K-Ras. In embodiments, the Switch II GTPase protein is H-Ras. In embodiments, the Switch II GTPase protein is N-Ras. In embodiments, the Switch II GTPase protein is E-Ras. In embodiments, the Switch II GTPase protein is RASD1. In embodiments, the Switch II GTPase protein is Rhes. In embodiments, the Switch II GTPase protein is RASL11B. In embodiments, the Switch II GTPase protein is REM2. In embodiments, the Switch II GTPase protein is RHOH. In embodiments, the Switch II GTPase protein is RND3. In embodiments, the Switch II GTPase protein is RAB1A. In embodiments, the Switch II GTPase protein is RAB1B. In embodiments, the Switch II GTPase protein is RAB2A. In embodiments, the Switch II GTPase protein is RAB2B. In embodiments, the Switch II GTPase protein is GNAZ. In embodiments, the Switch II GTPase protein is LRRK2. In embodiments, the Switch II GTPase protein is a GTPase protein listed in Colicelli, J. Sci STKE, 2004(250), RE13, which is incorporated herein by reference in its entirety and for all purposes. In embodiments, the Switch II GTPase protein is ARF1 (e.g., UniProt P84077). In embodiments, the Switch II GTPase protein is ARF3 (e.g., UniProt P61204). In embodiments, the Switch II GTPase protein is ARF4 (e.g., UniProt P18085). In embodiments, the Switch II GTPase protein is ARF5 (e.g., UniProt P84085). In embodiments, the Switch II GTPase protein is ARF6 (e.g., UniProt P62330). In embodiments, the Switch II GTPase protein is TRIM23 (e.g., UniProt P36406). In embodiments, the Switch II GTPase protein is ARL1 (e.g., UniProt P40616). In embodiments, the Switch II GTPase protein is ARL2 (e.g., UniProt P36404). In embodiments, the Switch II GTPase protein is ARL3 (e.g., UniProt P36405). In embodiments, the Switch II GTPase protein is ARL4A (e.g., UniProt P40617). In embodiments, the Switch II GTPase protein is ARL4B. In embodiments, the Switch II GTPase protein is ARL5. In embodiments, the Switch II GTPase protein is ARL6 (e.g., UniProt Q9H0F7). In embodiments, the Switch II GTPase protein is ARL7. In embodiments, the Switch II GTPase protein is ARL8. In embodiments, the Switch II GTPase protein is ARL9 (e.g., UniProt Q6T311). In embodiments, the Switch II GTPase protein is ARL12. In embodiments, the Switch II GTPase protein is ARL11 (e.g., UniProt Q969Q4). In embodiments, the Switch II GTPase protein is ARF7. In embodiments, the Switch II GTPase protein is 339231 (e.g., UniProt Q0P5N6). In embodiments, the Switch II GTPase protein is DKFZp761. In embodiments, the Switch II GTPase protein is ARFRP1 (e.g., UniProt Q13795). In embodiments, the Switch II GTPase protein is ARFRP2 (e.g., UniProt Q9NXU5). In embodiments, the Switch II GTPase protein is ARL10A (e.g., UniProt Q8N8L6). In embodiments, the Switch II GTPase protein is ARL10B (e.g., UniProt Q96BM9). In embodiments, the Switch II GTPase protein is ARL10C. In embodiments, the Switch II GTPase protein is 344988. In embodiments, the Switch II GTPase protein is SARA1 (e.g., UniProt Q6FID4). In embodiments, the Switch II GTPase protein is SARA2.

The term “Switch II GTPase protein serine residue” as used herein refers to a serine residue of a Switch II GTPase protein. In embodiments, the Switch II GTPase protein serine residue is a serine residue corresponding to the 12 position of a Ras protein (e.g., K-Ras, H-Ras, or N-Ras). In embodiments, the Switch II GTPase protein serine residue is a natural Switch II GTPase protein serine residue. In embodiments, the Switch II GTPase protein serine residue is a mutant Switch II GTPase protein serine residue. In embodiments, the mutant Switch II GTPase protein serine residue is K-Ras(G12S), H-Ras(G12S), or N-Ras(G12S).

The term “Switch II GTPase protein threonine residue” as used herein refers to a threonine residue of a Switch II GTPase protein. In embodiments, the Switch II GTPase protein threonine residue is a threonine residue corresponding to the 12 position of a Ras protein (e.g., K-Ras, H-Ras, or N-Ras). In embodiments, the Switch II GTPase protein threonine residue is a natural Switch II GTPase protein threonine residue. In embodiments, the Switch II GTPase protein threonine residue is a mutant Switch II GTPase protein threonine residue. In embodiments, the mutant Switch II GTPase protein threonine residue is K-Ras(G12T), H-Ras(G12T), or N-Ras(G12T).

The term “Switch II GTPase protein aspartate residue” as used herein refers to an aspartate residue of a Switch II GTPase protein. In embodiments, the Switch II GTPase protein aspartate residue is an aspartate residue corresponding to the 12 position of a Ras protein (e.g., K-Ras, H-Ras, or N-Ras). In embodiments, the Switch II GTPase protein aspartate residue is an aspartate residue corresponding to the 13 position of a Ras protein (e.g., K-Ras, H-Ras, or N-Ras). In embodiments, the Switch II GTPase protein aspartate residue is a natural Switch II GTPase protein aspartate residue. In embodiments, the Switch II GTPase protein aspartate residue is a mutant Switch II GTPase protein aspartate residue. In embodiments, the mutant Switch II GTPase protein aspartate residue is aspartate residue 12 of K-Ras(G12D), H-Ras(G12D), or N-Ras(G12D). In embodiments, the mutant Switch II GTPase protein aspartate residue is aspartate residue 13 of K-Ras(G13D), H-Ras(G13D), or N-Ras(G13D).

The term “Switch II GTPase protein glutamate residue” as used herein refers to a glutamate residue of a Switch II GTPase protein. In embodiments, the Switch II GTPase protein glutamate residue is a glutamate residue corresponding to the 12 position of a Ras protein (e.g., K-Ras, H-Ras, or N-Ras). In embodiments, the Switch II GTPase protein glutamate residue is a natural Switch II GTPase protein glutamate residue. In embodiments, the Switch II GTPase protein glutamate residue is a mutant Switch II GTPase protein glutamate residue. In embodiments, the mutant Switch II GTPase protein glutamate residue is glutamate residue 12 of K-Ras(G12D), H-Ras(G12D), or N-Ras(G12D).

The term “Ras” refers to one or more of the family of human Ras GTPase proteins (e.g. K-Ras, H-Ras, N-Ras), including homologs, isoforms, and functional fragments thereof.

The term “K-Ras” refers to the protein that in humans is encoded by the KRAS gene. The K-Ras protein is a GTPase, which converts guanosine triphosphate to guanosine diphosphate. A mutation in the K-Ras protein (e.g., an amino acid substitution) can result in various malignancies (e.g., lung adenocarcinoma, pancreatic cancer, or colorectal cancer). The term “K-Ras” may refer to the nucleotide sequence or protein sequence of human KRAS (e.g., Entrez 3845, UniProt P01116, RefSeq NM_004985.4, RefSeq NM_033360.3, RefSeq NP_004976.2, or RefSeq NP_203524.1). In embodiments, K-Ras has the following amino acid sequence:

(SEQ ID NO: 1)
MTEYKLVVVGAGGVGKSALTIQLIQNHFVDEYDPTIEDSYRKQVVIDGE
TCLLDILDTAGQEEYSAMRDQYMRTGEGFLCVFAINNTKSFEDIHHYRE
QIKRVKDSEDVPMVLVGNKCDLPSRTVDTKQAQDLARSYGIPFIETSAK
TRQRVEDAFYTLVREIRQYRLKKISKEEKTPGCVKIKKCIIM.

In embodiments, K-Ras has the following amino acid sequence:

(SEQ ID NO: 56)
MTEYKLVVVGAGGVGKSALTIQLIQNHFVDEYDPTIEDSYRKQVVIDGE
TCLLDILDTAGQEEYSAMRDQYMRTGEGFLCVFAINNTKSFEDIHHYRE
QIKRVKDSEDVPMVLVGNKCDLPSRTVDTKQAQDLARSYGIPFIETSAK
TRQGVDDAFYTLVREIRKHKEKMSKDGKKKKKKSKTKCVIM.

The term “H-Ras” refers to the enzyme that in humans is encoded by the HRAS gene. The H-Ras protein is a GTPase, which converts guanosine triphosphate to guanosine diphosphate. Mutations in the H-Ras protein (e.g., an amino acid substitution) can result in various malignancies (e.g., bladder cancer, thyroid cancer, salivary duct carcinoma, epithelial carcinoma, or kidney cancer). The term “H-Ras” may refer to the nucleotide sequence or protein sequence of human HRAS (e.g., Entrez 3265, UniProt P01112, RefSeq NM_001130442.2, RefSeq NM_001318054.1, RefSeq NM_005343.3, RefSeq NM_00176795.4, RefSeq NP_001123914.1, RefSeq NP_001304983.1, RefSeq NP_005334.1, or RefSeq NP_789765.1). In embodiments, H-Ras has the following amino acid sequence:

(SEQ ID NO: 2)
MTEYKLVVVGAGGVGKSALTIQLIQNHFVDEYDPTIEDSYRKQVVIDGE
TCLLDILDTAGQEEYSAMRDQYMRTGEGFLCVFAINNTKSFEDIHQYRE
QIKRVKDSDDVPMVLVGNKCDLAARTVESRQAQDLARSYGIPYIETSAK
TRQGVEDAFYTLVREIRQHKLRKLNPPDESGPGCMSCKCVLS.

The term “N-Ras” refers to the enzyme that in humans is encoded by the NRAS gene. The N-Ras protein is a GTPase, which converts guanosine triphosphate to guanosine diphosphate. The term “N-Ras” may refer to the nucleotide sequence or protein sequence of human NRAS (e.g., Entrez 4893, UniProt P01111, RefSeq NM_002524.4, or RefSeq NP_002515.1). In embodiments, N-Ras has the following amino acid sequence:

(SEQ ID NO: 3)
MTEYKLVVVGAGGVGKSALTIQLIQNHFVDEYDPTIEDSYRKQVVIDGE
TCLLDILDTAGQEEYSAMRDQYMRTGEGFLCVFAINNSKSFADINLYRE
QIKRVKDSDDVPMVLVGNKCDLPTRTVDTKQAHELAKSYGIPFIETSAK
TRQGVEDAFYTLVREIRQYRMKKLNSSDDGTQGCMGLPCVVM.

In embodiments, the Switch II Binding Pocket is bound at least in part by one or more of V7, V9, G10, P34, T58, G60, Q61, E62, E63, R68, Y71, M72, Y96, Q99, and/or 1100 of K-Ras or equivalent residues in homologous, related (e.g., H-Ras or N-Ras), or mutant Ras proteins. A compound as described herein (including embodiments, examples, and figures), which binds to amino acids that form or contacts amino acids that form the Switch II Binding Pocket is a “Switch II Binding Pocket binding compound” and a moiety of a compound that binds to amino acids that form or contacts amino acids that form the Switch II Binding Pocket is a “Switch II Binding Pocket binding moiety”.

In embodiments, a Switch II Binding Pocket binding compound or Switch II Binding Pocket binding moiety binds or contacts one amino acid that forms the Switch II Binding Pocket. In embodiments, a Switch II Binding Pocket binding compound or Switch II Binding Pocket binding moiety binds or contacts multiple amino acids that form the Switch II Binding Pocket. In embodiments, a Switch II Binding Pocket binding compound or Switch II-Binding Pocket binding moiety binds or contacts one amino acid selected from amino acids in a mutant K-Ras (e.g., K-Ras(G12S), K-Ras(G13S), or K-Ras(G12T)), related Ras (e.g., H-Ras, H-Ras(G12S), H-Ras(G13S), H-Ras(G12T), N-Ras, N-Ras(G12S), N-Ras(G13S), or N-Ras(G12T)), or homolog of K-Ras corresponding to K-Ras residues V7, V9, G10, P34, T58, G60, Q61, E62, E63, R68, Y71, M72, Y96, Q99, and 1100. In embodiments, a Switch II Binding Pocket binding compound or Switch II Binding Pocket binding moiety binds or contacts multiple K-Ras amino acids selected from amino acids in a mutant K-Ras (e.g., K-Ras(G12S), K-Ras(G13S), or K-Ras(G12T)), related Ras (e.g., H-Ras, H-Ras(G12S), H-Ras(G13S), H-Ras(G12T), N-Ras, N-Ras(G12S), N-Ras(G13S), or N-Ras(G12T)), or homolog of K-Ras corresponding to K-Ras residues V7, V9, G10, P34, T58, G60, Q61, E62, E63, R68, Y71, M72, Y96, Q99, and 1100. In embodiments, a Switch II Binding Pocket binding compound or Switch II Binding Pocket binding moiety binds or contacts two K-Ras amino acids selected from amino acids in a mutant K-Ras (e.g., K-Ras(G12S), K-Ras(G13S), or K-Ras(G12T)), related Ras (e.g., H-Ras, H-Ras(G12S), H-Ras(G13S), H-Ras(G12T), N-Ras, N-Ras(G12S), N-Ras(G13S), or N-Ras(G12T)), or homolog of K-Ras corresponding to K-Ras residues V7, V9, G10, P34, T58, G60, Q61, E62, E63, R68, Y71, M72, Y96, Q99, and 1100. In embodiments, a Switch II Binding Pocket binding compound or Switch II Binding Pocket binding moiety binds or contacts three K-Ras amino acids selected from amino acids in a mutant K-Ras (e.g., K-Ras(G12S), K-Ras(G13S), or K-Ras(G12T)), related Ras (e.g., H-Ras, H-Ras(G12S), H-Ras(G13S), H-Ras(G12T), N-Ras, N-Ras(G12S), N-Ras(G13S), or N-Ras(G12T)), or homolog of K-Ras corresponding to K-Ras residues V7, V9, G10, P34, T58, G60, Q61, E62, E63, R68, Y71, M72, Y96, Q99, and 1100. In embodiments, a Switch II Binding Pocket binding compound or Switch II Binding Pocket binding moiety binds or contacts four K-Ras amino acids selected from amino acids in a mutant K-Ras (e.g., K-Ras(G12S), K-Ras(G13S), or K-Ras(G12T)), related Ras (e.g., H-Ras, H-Ras(G12S), H-Ras(G13S), H-Ras(G12T), N-Ras, N-Ras(G12S), N-Ras(G13S), or N-Ras(G12T)), or homolog of K-Ras corresponding to K-Ras residues V7, V9, G10, P34, T58, G60, Q61, E62, E63, R68, Y71, M72, Y96, Q99, and 1100. In embodiments, a Switch II Binding Pocket binding compound or Switch II Binding Pocket binding moiety binds or contacts five K-Ras amino acids selected from amino acids in a mutant K-Ras (e.g., K-Ras(G12S), K-Ras(G13S), or K-Ras(G12T)), related Ras (e.g., H-Ras, H-Ras(G12S), H-Ras(G13S), H-Ras(G12T), N-Ras, N-Ras(G12S), N-Ras(G13S), or N-Ras(G12T)), or homolog of K-Ras corresponding to K-Ras residues V7, V9, G10, P34, T58, G60, Q61, E62, E63, R68, Y71, M72, Y96, Q99, and 1100. In embodiments, a Switch II Binding Pocket binding compound or Switch II Binding Pocket binding moiety binds or contacts six K-Ras amino acids selected from amino acids in a mutant K-Ras (e.g., K-Ras(G12S), K-Ras(G13S), or K-Ras(G12T)), related Ras (e.g., H-Ras, H-Ras(G12S), H-Ras(G13S), H-Ras(G12T), N-Ras, N-Ras(G12S), N-Ras(G13S), or N-Ras(G12T)), or homolog of K-Ras corresponding to K-Ras residues V7, V9, G10, P34, T58, G60, Q61, E62, E63, R68, Y71, M72, Y96, Q99, and 1100. In embodiments, a Switch II Binding Pocket binding compound or Switch II Binding Pocket binding moiety binds or contacts seven K-Ras amino acids selected from amino acids in a mutant K-Ras (e.g., K-Ras(G12S), K-Ras(G13S), or K-Ras(G12T)), related Ras (e.g., H-Ras, H-Ras(G12S), H-Ras(G13S), H-Ras(G12T), N-Ras, N-Ras(G12S), N-Ras(G13S), or N-Ras(G12T)), or homolog of K-Ras corresponding to K-Ras residues V7, V9, G10, P34, T58, G60, Q61, E62, E63, R68, Y71, M72, Y96, Q99, and 1100. In embodiments, a Switch II Binding Pocket binding compound or Switch II Binding Pocket binding moiety binds or contacts eight K-Ras amino acids selected from amino acids in a mutant K-Ras (e.g., K-Ras(G12S), K-Ras(G13S), or K-Ras(G12T)), related Ras (e.g., H-Ras, H-Ras(G12S), H-Ras(G13S), H-Ras(G12T), N-Ras, N-Ras(G12S), N-Ras(G13S), or N-Ras(G12T)), or homolog of K-Ras corresponding to K-Ras residues V7, V9, G10, P34, T58, G60, Q61, E62, E63, R68, Y71, M72, Y96, Q99, and 1100. In embodiments, a Switch II Binding Pocket binding compound or Switch II Binding Pocket binding moiety binds or contacts nine K-Ras amino acids selected from amino acids in a mutant K-Ras (e.g., K-Ras(G12S), K-Ras(G13S), or K-Ras(G12T)), related Ras (e.g., H-Ras, H-Ras(G12S), H-Ras(G13S), H-Ras(G12T), N-Ras, N-Ras(G12S), N-Ras(G13S), or N-Ras(G12T)), or homolog of K-Ras corresponding to K-Ras residues V7, V9, G10, P34, T58, G60, Q61, E62, E63, R68, Y71, M72, Y96, Q99, and 1100. In embodiments, a Switch II Binding Pocket binding compound or Switch II Binding Pocket binding moiety binds or contacts ten K-Ras amino acids selected from amino acids in a mutant K-Ras (e.g., K-Ras(G12S), K-Ras(G13S), or K-Ras(G12T)), related Ras (e.g., H-Ras, H-Ras(G12S), H-Ras(G13S), H-Ras(G12T), N-Ras, N-Ras(G12S), N-Ras(G13S), or N-Ras(G12T)), or homolog of K-Ras corresponding to K-Ras residues V7, V9, G10, P34, T58, G60, Q61, E62, E63, R68, Y71, M72, Y96, Q99, and 1100. In embodiments, a Switch II Binding Pocket binding compound or Switch II Binding Pocket binding moiety binds or contacts eleven K-Ras amino acids selected from amino acids in a mutant K-Ras (e.g., K-Ras(G12S), K-Ras(G13S), or K-Ras(G12T)), related Ras (e.g., H-Ras, H-Ras(G12S), H-Ras(G13S), H-Ras(G12T), N-Ras, N-Ras(G12S), N-Ras(G13S), or N-Ras(G12T)), or homolog of K-Ras corresponding to K-Ras residues V7, V9, G10, P34, T58, G60, Q61, E62, E63, R68, Y71, M72, Y96, Q99, and 1100. In embodiments, a Switch II Binding Pocket binding compound or Switch II Binding Pocket binding moiety binds or contacts twelve K-Ras amino acids selected from amino acids in a mutant K-Ras (e.g., K-Ras(G12S), K-Ras(G13S), or K-Ras(G12T)), related Ras (e.g., H-Ras, H-Ras(G12S), H-Ras(G13S), H-Ras(G12T), N-Ras, N-Ras(G12S), N-Ras(G13S), or N-Ras(G12T)), or homolog of K-Ras corresponding to K-Ras residues V7, V9, G10, P34, T58, G60, Q61, E62, E63, R68, Y71, M72, Y96, Q99, and 1100. In embodiments, a Switch II Binding Pocket binding compound or Switch II Binding Pocket binding moiety binds or contacts thirteen K-Ras amino acids selected from amino acids in a mutant K-Ras (e.g., K-Ras(G12S), K-Ras(G13S), or K-Ras(G12T)), related Ras (e.g., H-Ras, H-Ras(G12S), H-Ras(G13S), H-Ras(G12T), N-Ras, N-Ras(G12S), N-Ras(G13S), or N-Ras(G12T)), or homolog of K-Ras corresponding to K-Ras residues V7, V9, G10, P34, T58, G60, Q61, E62, E63, R68, Y71, M72, Y96, Q99, and 1100. In embodiments, a Switch II Binding Pocket binding compound or Switch II Binding Pocket binding moiety binds or contacts fourteen K-Ras amino acids selected from amino acids in a mutant K-Ras (e.g., K-Ras(G12S), K-Ras(G13S), or K-Ras(G12T)), related Ras (e.g., H-Ras, H-Ras(G12S), H-Ras(G13S), H-Ras(G12T), N-Ras, N-Ras(G12S), N-Ras(G13S), or N-Ras(G12T)), or homolog of K-Ras corresponding to K-Ras residues V7, V9, G10, P34, T58, G60, Q61, E62, E63, R68, Y71, M72, Y96, Q99, and 1100. In embodiments, a Switch II Binding Pocket binding compound or Switch II Binding Pocket binding moiety binds or contacts fifteen K-Ras amino acids selected from amino acids in a mutant K-Ras (e.g., K-Ras(G12S), K-Ras(G13S), or K-Ras(G12T)), related Ras (e.g., H-Ras, H-Ras(G12S), H-Ras(G13S), H-Ras(G12T), N-Ras, N-Ras(G12S), N-Ras(G13S), or N-Ras(G12T)), or homolog of K-Ras corresponding to K-Ras residues V7, V9, G10, P34, T58, G60, Q61, E62, E63, R68, Y71, M72, Y96, Q99, and 1100.

In embodiments, a Switch II Binding Pocket binding compound or Switch II-Binding Pocket binding moiety binds or contacts one amino acid selected from amino acids in a mutant K-Ras (e.g., K-Ras(G12D), K-Ras(G13D), or K-Ras(G12E)), related Ras (e.g., H-Ras, H-Ras(G12D), H-Ras(G13D), H-Ras(G12E), N-Ras, N-Ras(G12D), N-Ras(G13D), or N-Ras(G12E)), or homolog of K-Ras corresponding to K-Ras residues V7, V9, G10, P34, T58, G60, Q61, E62, E63, R68, Y71, M72, Y96, Q99, and 1100. In embodiments, a Switch II Binding Pocket binding compound or Switch II Binding Pocket binding moiety binds or contacts multiple K-Ras amino acids selected from amino acids in a mutant K-Ras (e.g., K-Ras(G12D), K-Ras(G13D), or K-Ras(G12E)), related Ras (e.g., H-Ras, H-Ras(G12D), H-Ras(G13D), H-Ras(G12E), N-Ras, N-Ras(G12D), N-Ras(G13D), or N-Ras(G12E)), or homolog of K-Ras corresponding to K-Ras residues V7, V9, G10, P34, T58, G60, Q61, E62, E63, R68, Y71, M72, Y96, Q99, and 1100. In embodiments, a Switch II Binding Pocket binding compound or Switch II Binding Pocket binding moiety binds or contacts two K-Ras amino acids selected from amino acids in a mutant K-Ras (e.g., K-Ras(G12D), K-Ras(G13D), or K-Ras(G12E)), related Ras (e.g., H-Ras, H-Ras(G12D), H-Ras(G13D), H-Ras(G12E), N-Ras, N-Ras(G12D), N-Ras(G13D), or N-Ras(G12E)), or homolog of K-Ras corresponding to K-Ras residues V7, V9, G10, P34, T58, G60, Q61, E62, E63, R68, Y71, M72, Y96, Q99, and 1100. In embodiments, a Switch II Binding Pocket binding compound or Switch II Binding Pocket binding moiety binds or contacts three K-Ras amino acids selected from amino acids in a mutant K-Ras (e.g., K-Ras(G12D), K-Ras(G13D), or K-Ras(G12E)), related Ras (e.g., H-Ras, H-Ras(G12D), H-Ras(G13D), H-Ras(G12E), N-Ras, N-Ras(G12D), N-Ras(G13D), or N-Ras(G12E)), or homolog of K-Ras corresponding to K-Ras residues V7, V9, G10, P34, T58, G60, Q61, E62, E63, R68, Y71, M72, Y96, Q99, and 1100. In embodiments, a Switch II Binding Pocket binding compound or Switch II Binding Pocket binding moiety binds or contacts four K-Ras amino acids selected from amino acids in a mutant K-Ras(G12D), K-Ras(G13D), or K-Ras(G12E)), related Ras (e.g., H-Ras, H-Ras(G12D), H-Ras(G13D), H-Ras(G12E), N-Ras, N-Ras(G12D), N-Ras(G13D), or N-Ras(G12E)), or homolog of K-Ras corresponding to K-Ras residues V7, V9, G10, P34, T58, G60, Q61, E62, E63, R68, Y71, M72, Y96, Q99, and 1100. In embodiments, a Switch II Binding Pocket binding compound or Switch II Binding Pocket binding moiety binds or contacts five K-Ras amino acids selected from amino acids in a mutant K-Ras (e.g., K-Ras(G12D), K-Ras(G13D), or K-Ras(G12E)), related Ras (e.g., H-Ras, H-Ras(G12D), H-Ras(G13D), H-Ras(G12E), N-Ras, N-Ras(G12D), N-Ras(G13D), or N-Ras(G12E)), or homolog of K-Ras corresponding to K-Ras residues V7, V9, G10, P34, T58, G60, Q61, E62, E63, R68, Y71, M72, Y96, Q99, and 1100. In embodiments, a Switch II Binding Pocket binding compound or Switch II Binding Pocket binding moiety binds or contacts six K-Ras amino acids selected from amino acids in a mutant K-Ras (e.g., K-Ras(G12D), K-Ras(G13D), or K-Ras(G12E)), related Ras (e.g., H-Ras, H-Ras(G12D), H-Ras(G13D), H-Ras(G12E), N-Ras, N-Ras(G12D), N-Ras(G13D), or N-Ras(G12E)), or homolog of K-Ras corresponding to K-Ras residues V7, V9, G10, P34, T58, G60, Q61, E62, E63, R68, Y71, M72, Y96, Q99, and 1100. In embodiments, a Switch II Binding Pocket binding compound or Switch II Binding Pocket binding moiety binds or contacts seven K-Ras amino acids selected from amino acids in a mutant K-Ras (e.g., K-Ras(G12D), K-Ras(G13D), or K-Ras(G12E)), related Ras (e.g., H-Ras, H-Ras(G12D), H-Ras(G13D), H-Ras(G12E), N-Ras, N-Ras(G12D), N-Ras(G13D), or N-Ras(G12E)), or homolog of K-Ras corresponding to K-Ras residues V7, V9, G10, P34, T58, G60, Q61, E62, E63, R68, Y71, M72, Y96, Q99, and 1100. In embodiments, a Switch II Binding Pocket binding compound or Switch II Binding Pocket binding moiety binds or contacts eight K-Ras amino acids selected from amino acids in a mutant K-Ras (e.g., K-Ras(G12D), K-Ras(G13D), or K-Ras(G12E)), related Ras (e.g., H-Ras, H-Ras(G12D), H-Ras(G13D), H-Ras(G12E), N-Ras, N-Ras(G12D), N-Ras(G13D), or N-Ras(G12E)), or homolog of K-Ras corresponding to K-Ras residues V7, V9, G10, P34, T58, G60, Q61, E62, E63, R68, Y71, M72, Y96, Q99, and 1100. In embodiments, a Switch II Binding Pocket binding compound or Switch II Binding Pocket binding moiety binds or contacts nine K-Ras amino acids selected from amino acids in a mutant K-Ras(G12D), K-Ras(G13D), or K-Ras(G12E)), related Ras (e.g., H-Ras, H-Ras(G12D), H-Ras(G13D), H-Ras(G12E), N-Ras, N-Ras(G12D), N-Ras(G13D), or N-Ras(G12E)), or homolog of K-Ras corresponding to K-Ras residues V7, V9, G10, P34, T58, G60, Q61, E62, E63, R68, Y71, M72, Y96, Q99, and 1100. In embodiments, a Switch II Binding Pocket binding compound or Switch II Binding Pocket binding moiety binds or contacts ten K-Ras amino acids selected from amino acids in a mutant K-Ras (e.g., K-Ras(G12D), K-Ras(G13D), or K-Ras(G12E)), related Ras (e.g., H-Ras, H-Ras(G12D), H-Ras(G13D), H-Ras(G12E), N-Ras, N-Ras(G12D), N-Ras(G13D), or N-Ras(G12E)), or homolog of K-Ras corresponding to K-Ras residues V7, V9, G10, P34, T58, G60, Q61, E62, E63, R68, Y71, M72, Y96, Q99, and 1100. In embodiments, a Switch II Binding Pocket binding compound or Switch II Binding Pocket binding moiety binds or contacts eleven K-Ras amino acids selected from amino acids in a mutant K-Ras (e.g., K-Ras(G12D), K-Ras(G13D), or K-Ras(G12E)), related Ras (e.g., H-Ras, H-Ras(G12D), H-Ras(G13D), H-Ras(G12E), N-Ras, N-Ras(G12D), N-Ras(G13D), or N-Ras(G12E)), or homolog of K-Ras corresponding to K-Ras residues V7, V9, G10, P34, T58, G60, Q61, E62, E63, R68, Y71, M72, Y96, Q99, and 1100. In embodiments, a Switch II Binding Pocket binding compound or Switch II Binding Pocket binding moiety binds or contacts twelve K-Ras amino acids selected from amino acids in a mutant K-Ras (e.g., K-Ras(G12D), K-Ras(G13D), or K-Ras(G12E)), related Ras (e.g., H-Ras, H-Ras(G12D), H-Ras(G13D), H-Ras(G12E), N-Ras, N-Ras(G12D), N-Ras(G13D), or N-Ras(G12E)), or homolog of K-Ras corresponding to K-Ras residues V7, V9, G10, P34, T58, G60, Q61, E62, E63, R68, Y71, M72, Y96, Q99, and 1100. In embodiments, a Switch II Binding Pocket binding compound or Switch II Binding Pocket binding moiety binds or contacts thirteen K-Ras amino acids selected from amino acids in a mutant K-Ras (e.g., K-Ras(G12D), K-Ras(G13D), or K-Ras(G12E)), related Ras (e.g., H-Ras, H-Ras(G12D), H-Ras(G13D), H-Ras(G12E), N-Ras, N-Ras(G12D), N-Ras(G13D), or N-Ras(G12E)), or homolog of K-Ras corresponding to K-Ras residues V7, V9, G10, P34, T58, G60, Q61, E62, E63, R68, Y71, M72, Y96, Q99, and 1100. In embodiments, a Switch II Binding Pocket binding compound or Switch II Binding Pocket binding moiety binds or contacts fourteen K-Ras amino acids selected from amino acids in a mutant K-Ras (e.g., K-Ras(G12D), K-Ras(G13D), or K-Ras(G12E)), related Ras (e.g., H-Ras, H-Ras(G12D), H-Ras(G13D), H-Ras(G12E), N-Ras, N-Ras(G12D), N-Ras(G13D), or N-Ras(G12E)), or homolog of K-Ras corresponding to K-Ras residues V7, V9, G10, P34, T58, G60, Q61, E62, E63, R68, Y71, M72, Y96, Q99, and 1100. In embodiments, a Switch II Binding Pocket binding compound or Switch II Binding Pocket binding moiety binds or contacts fifteen K-Ras amino acids selected from amino acids in a mutant K-Ras (e.g., K-Ras(G12D), K-Ras(G13D), or K-Ras(G12E)), related Ras (e.g., H-Ras, H-Ras(G12D), H-Ras(G13D), H-Ras(G12E), N-Ras, N-Ras(G12D), N-Ras(G13D), or N-Ras(G12E)), or homolog of K-Ras corresponding to K-Ras residues V7, V9, G10, P34, T58, G60, Q61, E62, E63, R68, Y71, M72, Y96, Q99, and 1100.

The term “selective” or “selectivity” or the like in reference to a compound or agent refers to the compound's or agent's ability to cause an increase or decrease in activity of a particular molecular target (e.g., protein, enzyme, etc.) preferentially over one or more different molecular targets (e.g., a compound having selectivity toward mutant K-Ras(G12S) would preferentially inhibit K-Ras(G12S) over other K-Ras proteins (e.g., wild type K-Ras) or a compound having selectivity toward mutant K-Ras(G12D) would preferentially inhibit K-Ras(G12D) over other K-Ras proteins (e.g., wild type K-Ras)). In embodiments, a “Ras(G12S)-selective compound” refers to a compound (e.g., compound described herein) having selectivity towards Ras(G12S). In embodiments, a “Ras(G12D)-selective compound” refers to a compound (e.g., compound described herein) having selectivity towards Ras(G12D).

The term “β-lactone” is used in accordance with its plain ordinary meaning in organic chemistry and refers to a 4-membered heterocycloalkyl ring containing a —C(O)O— moiety. In embodiments, the β-lactone has the formula:

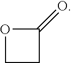

The term “β-lactam” is used in accordance with its plain ordinary meaning in organic chemistry and refers to a 4-membered heterocycloalkyl ring containing a —C(O)NR— moiety. In embodiments, the β-lactam has the formula:

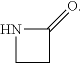

### II. Compounds

In an aspect is provided a compound, or a pharmaceutically acceptable salt thereof, having the formula:

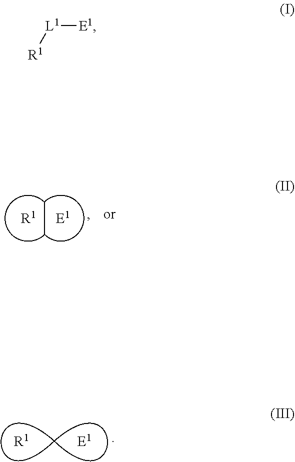

R¹ is a Switch II Binding Pocket binding moiety.
L¹ is a bond or divalent linker.
E¹ is an electrophilic moiety capable of forming a covalent bond with a Switch II GTPase protein serine residue or a Switch II GTPase protein threonine residue.

In embodiments, E¹ is an electrophilic moiety capable of forming a covalent bond with a Switch II GTPase protein serine residue. In embodiments, E¹ is an electrophilic moiety capable of forming a covalent bond with a Switch II GTPase protein threonine residue. In embodiments, the Switch II GTPase protein serine residue is a serine residue corresponding to the 12 position of a Ras protein (e.g., K-Ras, H-Ras, or N-Ras). In embodiments, the Switch II GTPase protein serine residue is a natural Switch II GTPase protein serine residue. In embodiments, the Switch II GTPase protein serine residue is a mutant Switch II GTPase protein serine residue. In embodiments, the the mutant Switch II GTPase protein serine residue is serine residue 12 of K-Ras(G12S), H-Ras(G12S), or N-Ras(G12S). In embodiments, the the mutant Switch II GTPase protein serine residue is serine residue 13 of K-Ras(G13S), H-Ras(G13S), or N-Ras(G13S). In embodiments, the Switch II GTPase protein threonine residue is a natural Switch II GTPase protein threonine residue. In embodiments, the Switch II GTPase protein threonine residue is a mutant Switch II GTPase protein threonine residue. In embodiments, the the mutant Switch II GTPase protein threonine residue is threonine residue 12 of K-Ras(G12T), H-Ras(G12T), or N-Ras(G12T).

In embodiments, the Switch II GTPase protein serine residue is a Ras protein serine residue. In embodiments, the Switch II GTPase protein serine residue is a K-Ras serine residue. In embodiments, the Switch II GTPase protein serine residue is an H-Ras serine residue. In embodiments, the Switch II GTPase protein serine residue is an N-Ras serine residue. In embodiments, the Switch II GTPase serine residue protein is an E-Ras serine residue. In embodiments, the Switch II GTPase protein serine residue is a RASD1 serine residue. In embodiments, the Switch II GTPase protein serine residue is a Rhes serine residue. In embodiments, the Switch II GTPase protein serine residue is a RASL11B serine residue. In embodiments, the Switch II GTPase protein serine residue is a REM2 serine residue. In embodiments, the Switch II GTPase protein serine residue is a RHOH serine residue. In embodiments, the Switch II GTPase protein serine residue is a RND3 serine residue. In embodiments, the Switch II GTPase protein serine residue is a RAB1A serine residue. In embodiments, the Switch II GTPase protein serine residue is a RAB1B serine residue. In embodiments, the Switch II GTPase protein serine residue is a RAB2A serine residue. In embodiments, the Switch II GTPase protein serine residue is a RAB2B serine residue. In embodiments, the Switch II GTPase protein serine residue is a GNAZ serine residue. In embodiments, the Switch II GTPase protein serine residue is a LRRK2 serine residue. In embodiments, the Switch II GTPase protein threonine residue is a LRRK2 threonine residue. In embodiments, the Switch II GTPase protein serine residue is a serine residue of a GTPase protein listed in Colicelli, J. Sci STKE, 2004(250), RE13, which is incorporated herein by reference in its entirety and for all purposes.

In embodiments, the Switch II GTPase protein threonine residue is a Ras protein threonine residue. In embodiments, the Switch II GTPase protein threonine residue is a K-Ras threonine residue. In embodiments, the Switch II GTPase protein threonine residue is an H-Ras threonine residue. In embodiments, the Switch II GTPase protein threonine residue is an N-Ras threonine residue.

In embodiments, E¹ is an electrophilic moiety capable of forming a covalent bond with a K-Ras serine residue. In embodiments, E¹ is an electrophilic moiety capable of forming a covalent bond with an H-Ras serine residue. In embodiments, E¹ is an electrophilic moiety capable of forming a covalent bond with an N-Ras serine residue. In embodiments, E¹ is an electrophilic moiety capable of forming a covalent bond with a K-Ras threonine residue. In embodiments, E¹ is an electrophilic moiety capable of forming a covalent bond with an H-Ras threonine residue. In embodiments, E¹ is an electrophilic moiety capable of forming a covalent bond with an N-Ras threonine residue. In embodiments, E¹ includes a β-lactone. In embodiments, E¹ includes a β-lactam.

In embodiments, the compound has the formula:

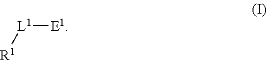

R¹, L¹, and E¹ are as described herein, including in embodiments.

In embodiments, the compound has the formula:

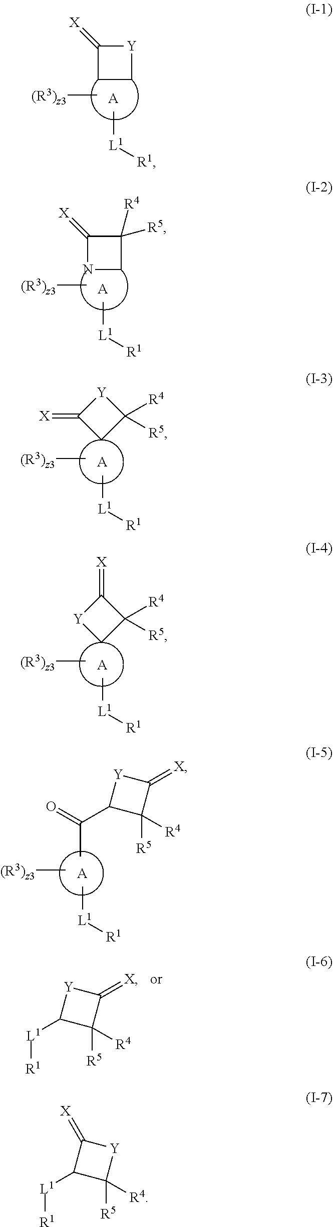

R¹ and L¹ are as described herein, including in embodiments.

Ring A is a cycloalkyl (e.g., C₃-C₈, C₃-C₆, C₄-C₆, or C₅-C₆) or heterocycloalkyl (e.g., 3 to 8 membered, 3 to 6 membered, 4 to 6 membered, 4 to 5 membered, or 5 to 6 membered).

X is O or S.

Y is O, S, or NR².

R² is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R³ is independently oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); two R³ substituents may optionally be joined to form a substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

The symbol z3 is an integer from 0 to 10.

R⁴ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R⁵ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, Ring A is a C₃-C₈ cycloalkyl. In embodiments, Ring A is a cycloalkyl. In embodiments, Ring A is a cyclobutyl. In embodiments, Ring A is a cyclopentyl. In embodiments, Ring A is a cyclohexyl. In embodiments, Ring A is a cycloheptyl. In embodiments, Ring A is a cyclooctyl. In embodiments, Ring A is a 3 to 8 membered heterocycloalkyl. In embodiments, Ring A is a 5 to 6 membered heterocycloalkyl. In embodiments, Ring A is a piperidinyl, pyrrolidinyl, or piperazinyl. In embodiments, Ring A is a piperidinyl. In embodiments, Ring A is a pyrrolidinyl. In embodiments, Ring A is a piperazinyl.

In embodiments, the compound has the formula:

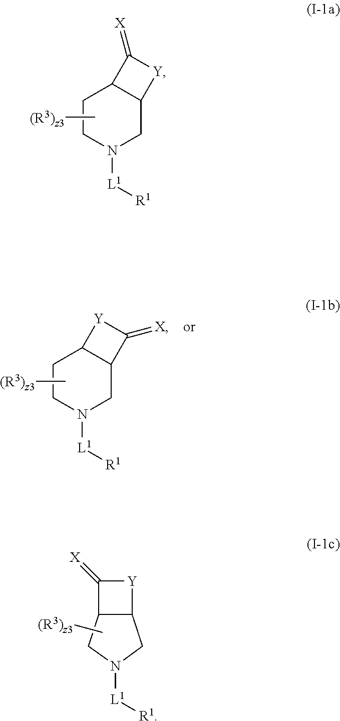

X, Y, R¹, L¹, R³, and z3 are as described herein, including in embodiments. In embodiments, the compound has the formula:

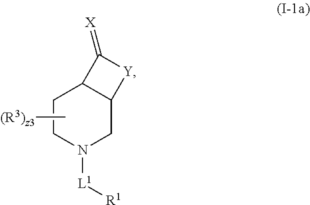

wherein X, Y, R¹, L¹, R³, and z3 are as described herein, including in embodiments. In embodiments, the compound has the formula:

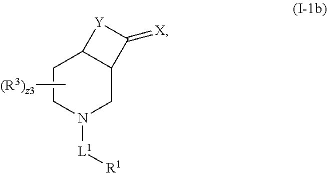

wherein X, Y, R¹, L¹, R³, and z3 are as described herein, including in embodiments. In embodiments, the compound has the formula:

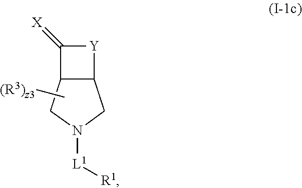

wherein X, Y, R¹, L¹, R³, and z3 are as described herein, including in embodiments.

In embodiments, the compound has the formula:

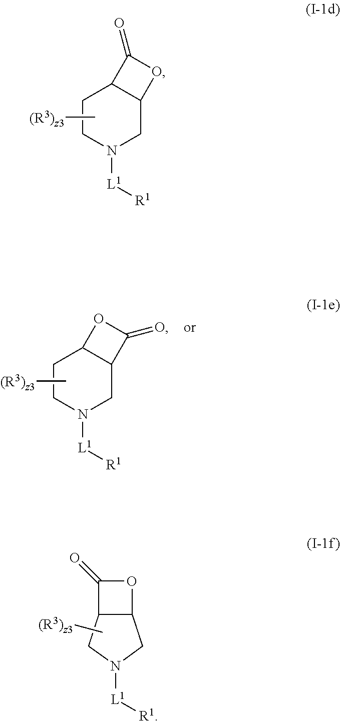

R¹, L¹, R³, and z3 are as described herein, including in embodiments. In embodiments, the compound has the formula:

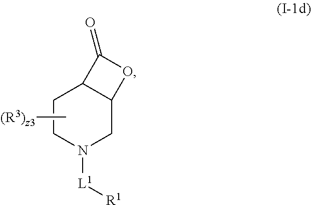

wherein R¹, L¹, R³, and z3 are as described herein, including in embodiments. In embodiments, the compound has the formula:

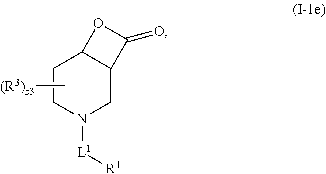

wherein R¹, L¹, R³, and z3 are as described herein, including in embodiments. In embodiments, the compound has the formula:

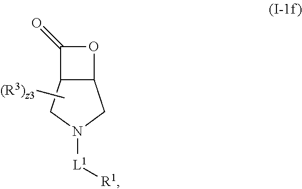

wherein R¹, L¹, R³, and z3 are as described herein, including in embodiments.

In embodiments, the compound has the formula:

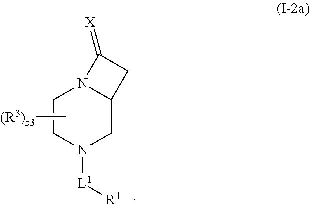

X, R¹, L¹, R³, and z3 are as described herein, including in embodiments.

In embodiments, the compound has the formula:

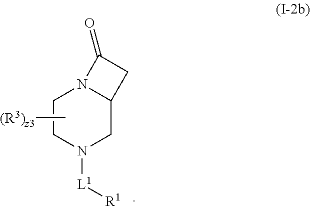

R¹, L¹, R³, and z3 are as described herein, including in embodiments.

In embodiments, the compound has the formula:

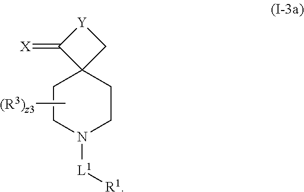

X, Y, R¹, L¹, R³, and z3 are as described herein, including in embodiments.

In embodiments, the compound has the formula:

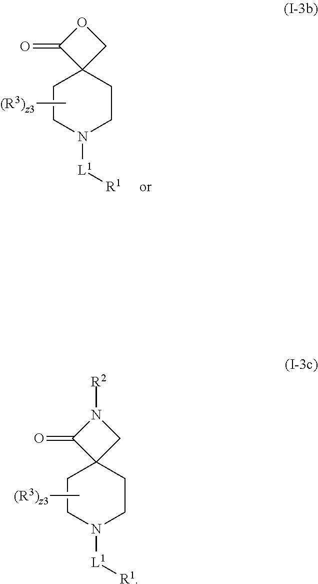

R¹, L¹, R², R³, and z3 are as described herein, including in embodiments. In embodiments, the compound has the formula:

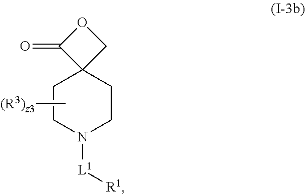

wherein R¹, L¹, R³, and z3 are as described herein, including in embodiments. In embodiments, the compound has the formula:

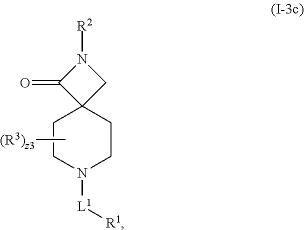

wherein R¹, L¹, R², R³, and z3 are as described herein, including in embodiments.

In embodiments, the compound has the formula:

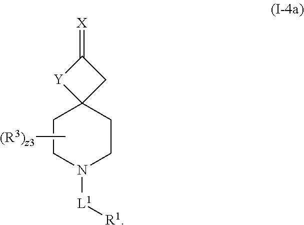

X, Y, R¹, L¹, R³, and z3 are as described herein, including in embodiments.

In embodiments, the compound has the formula:

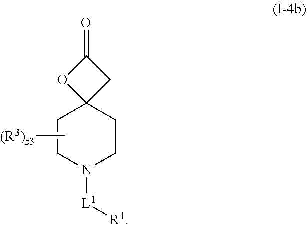

R¹, L¹, R³, and z3 are as described herein, including in embodiments.

In embodiments, the compound has the formula:

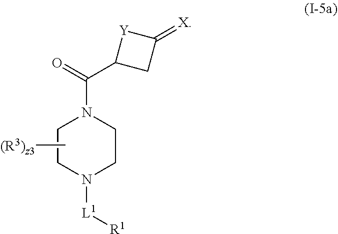

X, Y, R¹, L¹, R³, and z3 are as described herein, including in embodiments.

In embodiments, the compound has the formula:

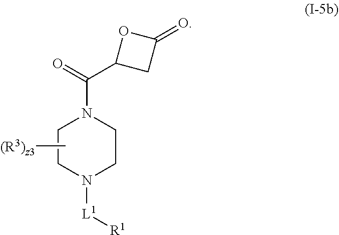

R¹, L¹, R³, and z3 are as described herein, including in embodiments.

In embodiments, the compound has the formula:

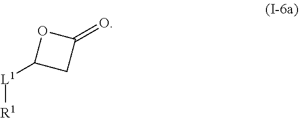

R¹ and L¹ are as described herein, including in embodiments.

In embodiments, the compound has the formula:

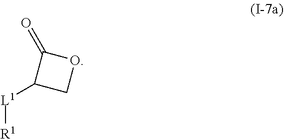

R¹ and L¹ are as described herein, including in embodiments.

In an aspect is provided a compound, or a pharmaceutically acceptable salt thereof, having the formula:

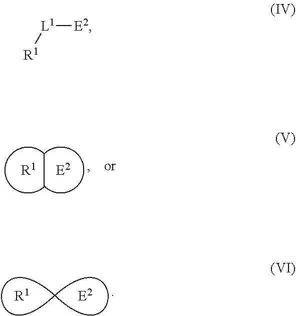

R¹ is a Switch II Binding Pocket binding moiety.

L¹ is a bond or divalent linker.

E² is an electrophilic moiety capable of forming a covalent bond with a Switch II GTPase protein aspartate residue or a Switch II GTPase protein glutamate residue.

In embodiments, E² is an electrophilic moiety capable of forming a covalent bond with a Switch II GTPase protein aspartate residue. In embodiments, E² is an electrophilic moiety capable of forming a covalent bond with a Switch II GTPase protein glutamate residue. In embodiments, the Switch II GTPase protein aspartate residue is a natural Switch II GTPase protein aspartate residue. In embodiments, the Switch II GTPase protein aspartate residue is a mutant Switch II GTPase protein aspartate residue. In embodiments, the Switch II GTPase protein aspartate residue is an aspartate residue corresponding to the 12 position of a Ras protein (e.g., K-Ras, H-Ras, or N-Ras). In embodiments, the the mutant Switch II GTPase protein aspartate residue is aspartate residue 12 of K-Ras(G12D), H-Ras(G12D), or N-Ras(G12D). In embodiments, the Switch II GTPase protein aspartate residue is an aspartate residue corresponding to the 13 position of a Ras protein (e.g., K-Ras, H-Ras, or N-Ras). In embodiments, the the mutant Switch II GTPase protein aspartate residue is aspartate residue 13 of K-Ras(G13D), H-Ras(G13D), or N-Ras(G13D). In embodiments, the Switch II GTPase protein glutamate residue is a glutamate residue corresponding to the 12 position of a Ras protein (e.g., K-Ras, H-Ras, or N-Ras). In embodiments, the the mutant Switch II GTPase protein glutamate residue is glutamate residue 12 of K-Ras(G12E), H-Ras(G12E), or N-Ras(G12E).

In embodiments, the Switch II GTPase protein aspartate residue is a Ras protein aspartate residue. In embodiments, the Switch II GTPase protein aspartate residue is a K-Ras aspartate residue. In embodiments, the Switch II GTPase protein aspartate residue is an H-Ras aspartate residue. In embodiments, the Switch II GTPase protein aspartate residue is an N-Ras aspartate residue. In embodiments, the Switch II GTPase protein aspartate residue is an ARF1 (e.g., UniProt P84077) aspartate residue. In embodiments, the Switch II GTPase protein aspartate residue is an ARF3 (e.g., UniProt P61204) aspartate residue. In embodiments, the Switch II GTPase protein aspartate residue is an ARF4 (e.g., UniProt P18085) aspartate residue. In embodiments, the Switch II GTPase protein aspartate residue is an ARF5 (e.g., UniProt P84085) aspartate residue. In embodiments, the Switch II GTPase protein aspartate residue is an ARF6 (e.g., UniProt P62330) aspartate residue. In embodiments, the Switch II GTPase protein aspartate residue is a TRIM23 (e.g., UniProt P36406) aspartate residue. In embodiments, the Switch II GTPase protein aspartate residue is an ARL1 (e.g., UniProt P40616) aspartate residue. In embodiments, the Switch II GTPase protein aspartate residue is an ARL2 (e.g., UniProt P36404) aspartate residue. In embodiments, the Switch II GTPase protein aspartate residue is an ARL3 (e.g., UniProt P36405) aspartate residue. In embodiments, the Switch II GTPase protein aspartate residue is an ARL4A (e.g., UniProt P40617) aspartate residue. In embodiments, the Switch II GTPase protein aspartate residue is an ARL4B aspartate residue. In embodiments, the Switch II GTPase protein aspartate residue is an ARL5 aspartate residue. In embodiments, the Switch II GTPase protein aspartate residue is an ARL6 (e.g., UniProt Q9H0F7) aspartate residue. In embodiments, the Switch II GTPase protein aspartate residue is an ARL7 aspartate residue. In embodiments, the Switch II GTPase protein aspartate residue is an ARL8 aspartate residue. In embodiments, the Switch II GTPase protein aspartate residue is an ARL9 (e.g., UniProt Q6T311) aspartate residue. In embodiments, the Switch II GTPase protein aspartate residue is an ARL12 aspartate residue. In embodiments, the Switch II GTPase protein aspartate residue is an ARL11 (e.g., UniProt Q969Q4) aspartate residue. In embodiments, the Switch II GTPase protein aspartate residue is an ARF7 aspartate residue. In embodiments, the Switch II GTPase protein aspartate residue is a 339231 (e.g., UniProt Q0P5N6) aspartate residue. In embodiments, the Switch II GTPase protein aspartate residue is a DKFZp761 aspartate residue. In embodiments, the Switch II GTPase protein aspartate residue is an ARFRP1 (e.g., UniProt Q13795) aspartate residue. In embodiments, the Switch II GTPase protein aspartate residue is an ARFRP2 (e.g., UniProt Q9NXU5) aspartate residue. In embodiments, the Switch II GTPase protein aspartate residue is an ARL10A (e.g., UniProt Q8N8L6) aspartate residue. In embodiments, the Switch II GTPase protein aspartate residue is an ARL10B (e.g., UniProt Q96BM9) aspartate residue. In embodiments, the Switch II GTPase protein aspartate residue is an ARL10C aspartate residue. In embodiments, the Switch II GTPase protein aspartate residue is a 344988 aspartate residue. In embodiments, the Switch II GTPase protein aspartate residue is a SARA1 (e.g., UniProt Q6FID4) aspartate residue. In embodiments, the Switch II GTPase protein aspartate residue is a SARA2 aspartate residue.

In embodiments, the Switch II GTPase protein glutamate residue is a Ras protein glutamate residue. In embodiments, the Switch II GTPase protein glutamate residue is a K-Ras glutamate residue. In embodiments, the Switch II GTPase protein glutamate residue is an H-Ras glutamate residue. In embodiments, the Switch II GTPase protein glutamate residue is an N-Ras glutamate residue.

In embodiments, E² is an electrophilic moiety capable of forming a covalent bond with a K-Ras aspartate residue. In embodiments, E² is an electrophilic moiety capable of forming a covalent bond with an H-Ras aspartate residue. In embodiments, E² is an electrophilic moiety capable of forming a covalent bond with an N-Ras aspartate residue. In embodiments, E² is an electrophilic moiety capable of forming a covalent bond with a K-Ras glutamate residue. In embodiments, E² is an electrophilic moiety capable of forming a covalent bond with an H-Ras glutamate residue. In embodiments, E² is an electrophilic moiety capable of forming a covalent bond with an N-Ras glutamate residue. In embodiments, E² includes a β-lactone. In embodiments, E² includes a β-lactam.

In embodiments, the compound has the formula:

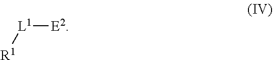

R¹, L¹, and E² are as described herein, including in embodiments.

In embodiments, the compound has the formula:

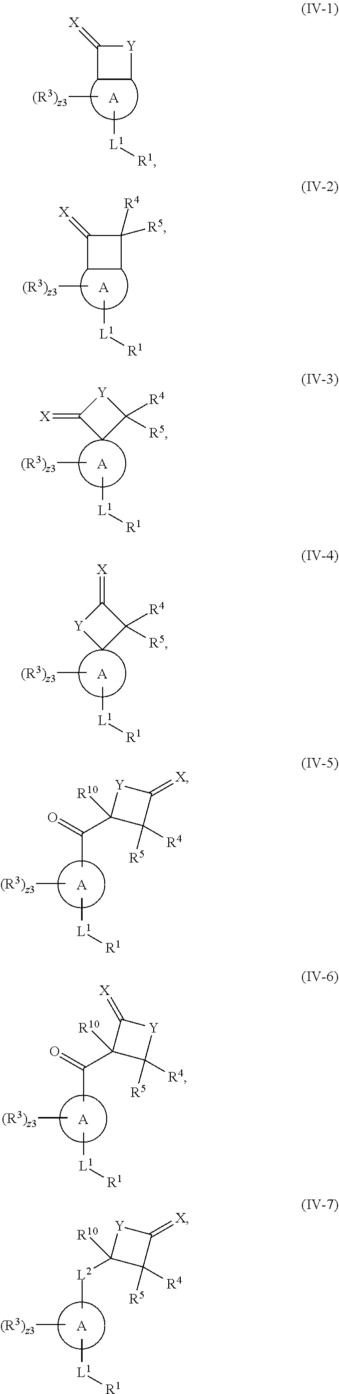

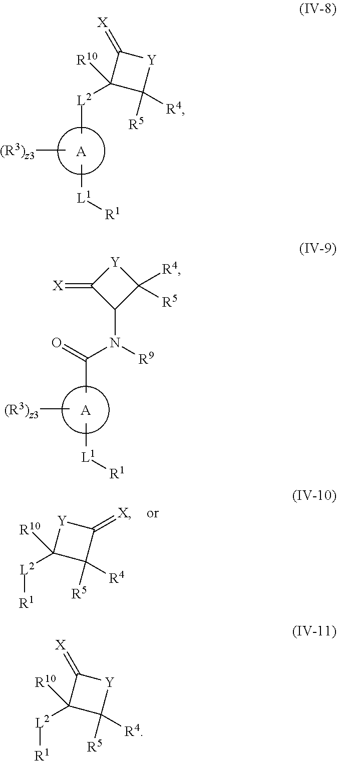

R¹ and L¹ are as described herein, including in embodiments.

Ring A is a cycloalkyl (e.g., C₃-C₈, C₃-C₆, C₄-C₆, or C₅-C₆) or heterocycloalkyl (e.g., 3 to 8 membered, 3 to 6 membered, 4 to 6 membered, 4 to 5 membered, or 5 to 6 membered).

L² is unsubstituted C₁-C₄ alkylene.

X is O or S.

Y is O, S, or NR².

R² is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R³ is independently oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); two R³ substituents may optionally be joined to form a substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

The symbol z3 is an integer from 0 to 10.

R⁴ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R⁵ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R⁹ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R¹⁰ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, Ring A is a C₃-C₈ cycloalkyl. In embodiments, Ring A is a cycloalkyl. In embodiments, Ring A is a cyclobutyl. In embodiments, Ring A is a cyclopentyl. In embodiments, Ring A is a cyclohexyl. In embodiments, Ring A is a cycloheptyl. In embodiments, Ring A is a cyclooctyl. In embodiments, Ring A is a 3 to 8 membered heterocycloalkyl. In embodiments, Ring A is a 5 to 6 membered heterocycloalkyl. In embodiments, Ring A is a piperidinyl, pyrrolidinyl, or piperazinyl. In embodiments, Ring A is a piperidinyl. In embodiments, Ring A is a pyrrolidinyl. In embodiments, Ring A is a piperazinyl.

In embodiments, the compound has the formula:

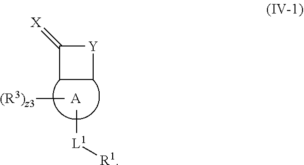

Ring A, X, Y, R¹, L¹, R³, and z3 are as described herein, including in embodiments.

In embodiments, the compound has the formula:

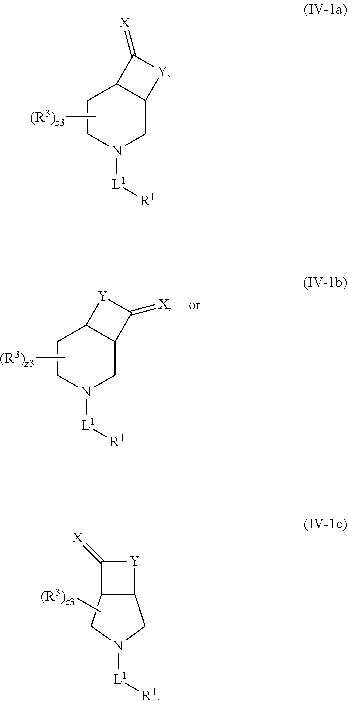

X, Y, R¹, L¹, R³, and z3 are as described herein, including in embodiments. In embodiments, the compound has the formula:

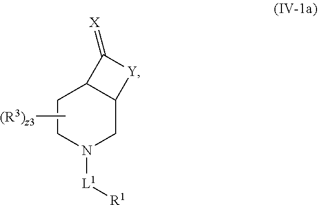

wherein X, Y, R¹, L¹, R³, and z3 are as described herein, including in embodiments. In embodiments, the compound has the formula:

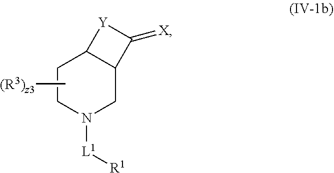

wherein X, Y, R¹, L¹, R³, and z3 are as described herein, including in embodiments. In embodiments, the compound has the formula:

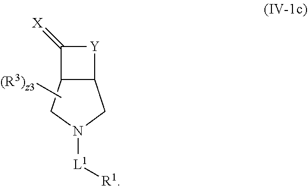

wherein X, Y, R¹, L¹, R³, and z3 are as described herein, including in embodiments.

In embodiments, the compound has the formula:

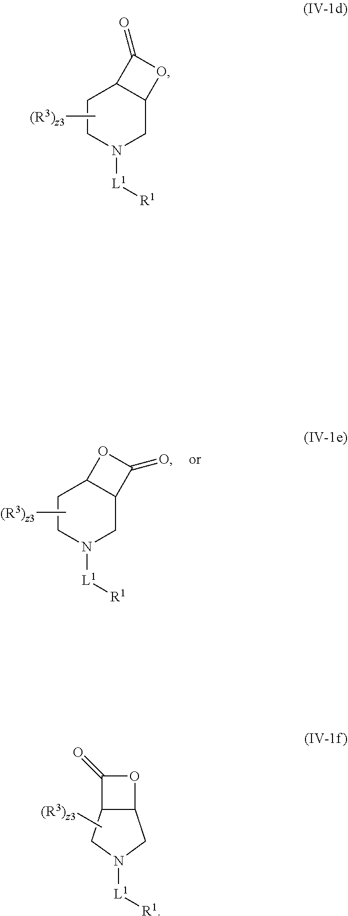

R¹, L¹, R³, and z3 are as described herein, including in embodiments. In embodiments, the compound has the formula:

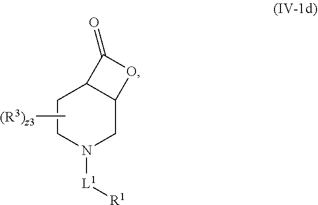

wherein R¹, L¹, R³, and z3 are as described herein, including in embodiments. In embodiments, the compound has the formula:

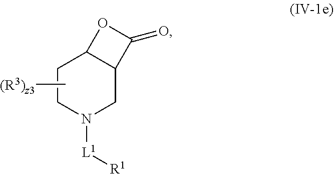

wherein R¹, L¹, R³, and z3 are as described herein, including in embodiments. In embodiments, the compound has the formula:

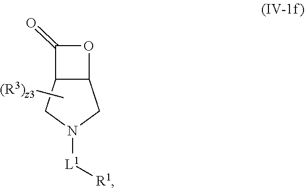

wherein R¹, L¹, R³, and z3 are as described herein, including in embodiments.

In embodiments, the compound has the formula:

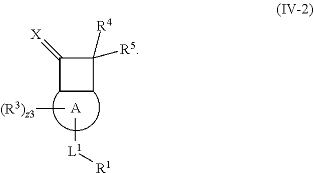

Ring A, X, R¹, L¹, R³, z3, R⁴, and R⁵ are as described herein, including in embodiments.

In embodiments, the compound has the formula:

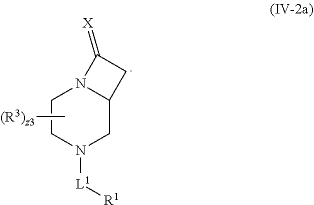

X, R¹, L¹, R³, and z3 are as described herein, including in embodiments.

In embodiments, the compound has the formula:

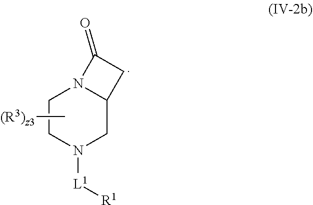

R¹, L¹, R³, and z3 are as described herein, including in embodiments.

In embodiments, the compound has the formula:

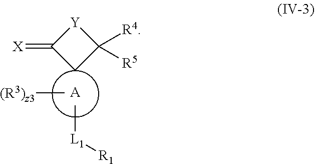

Ring A, X, Y, R¹, L¹, R³, z3, R⁴, and R⁵ are as described herein, including in embodiments.

In embodiments, the compound has the formula:

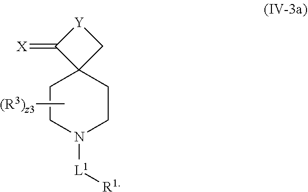

X, Y, R¹, L¹, R³, and z3 are as described herein, including in embodiments.

In embodiments, the compound has the formula:

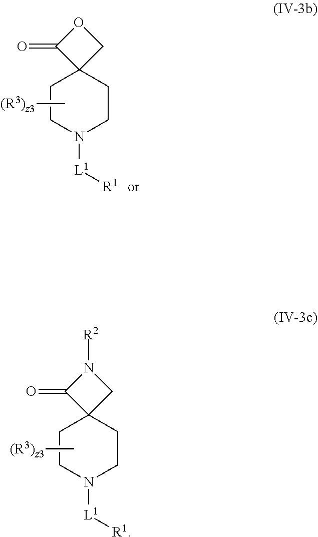

R¹, L¹, R², R³, and z3 are as described herein, including in embodiments. In embodiments, the compound has the formula:

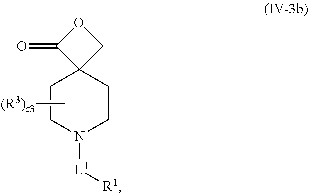

wherein R¹, L¹, R³, and z3 are as described herein, including in embodiments. In embodiments, the compound has the formula:

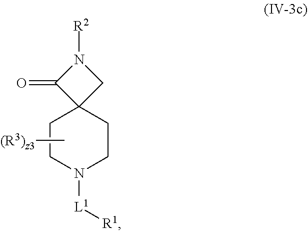

wherein R¹, L¹, R², R³, and z3 are as described herein, including in embodiments.

In embodiments, the compound has the formula:

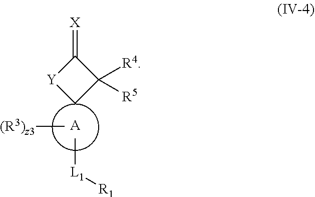

Ring A, X, Y, R¹, L¹, R³, z3, R⁴, and R⁵ are as described herein, including in embodiments.

In embodiments, the compound has the formula:

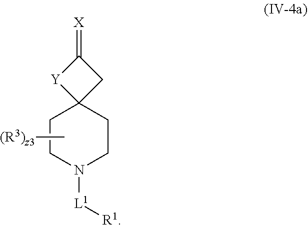

X, Y, R¹, L¹, R³, and z3 are as described herein, including in embodiments.

In embodiments, the compound has the formula:

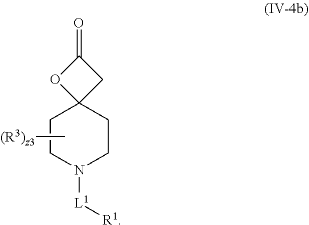

R¹, L¹, R³, and z3 are as described herein, including in embodiments.

In embodiments, the compound has the formula:

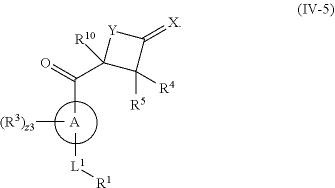

Ring A, X, Y, R¹, L¹, R³, z3, R⁴, R⁵, and R¹⁰ are as described herein, including in embodiments.

In embodiments, the compound has the formula:

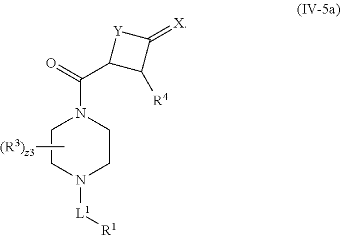

X, Y, R¹, L¹, R³, z3, and R⁴ are as described herein, including in embodiments.

In embodiments, the compound has the formula:

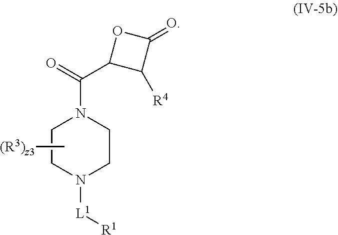

R¹, L¹, R³, z3, and R⁴ are as described herein, including in embodiments.

In embodiments, the compound has the formula:

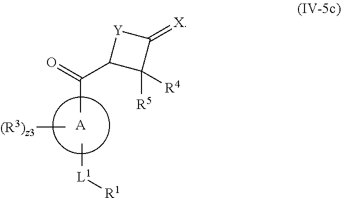

Ring A, X, Y, R¹, L¹, R³, z3, R⁴, and R⁵ are as described herein, including in embodiments.

In embodiments, the compound has the formula:

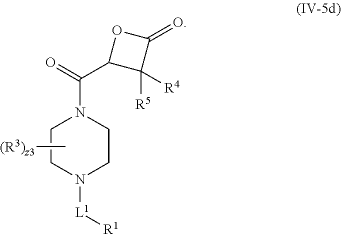

R¹, L¹, R³, z3, R⁴, and R⁵ are as described herein, including in embodiments.

In embodiments, the compound has the formula:

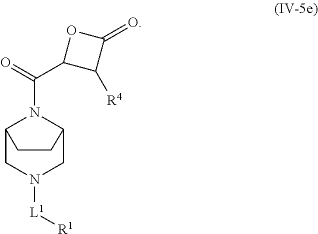

R¹, L¹, R⁴ are as described herein, including in embodiments.

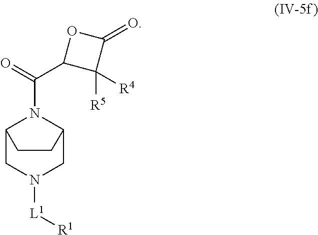

R¹, L¹, R⁴, and R⁵ are as described herein, including in embodiments.

In embodiments, the compound has the formula:

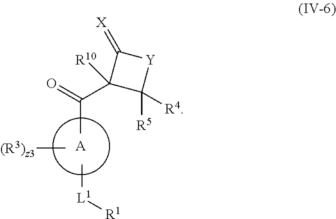

Ring A, X, Y, R¹, L¹, R³, z3, R⁴, R⁵, and R¹⁰ are as described herein, including in embodiments.

In embodiments, the compound has the formula:

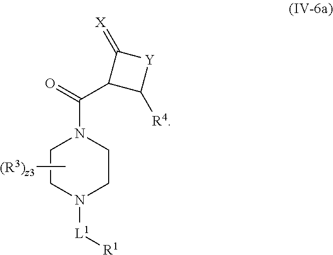

X, Y, R¹, L¹, R³, z3, and R⁴ are as described herein, including in embodiments.

In embodiments, the compound has the formula:

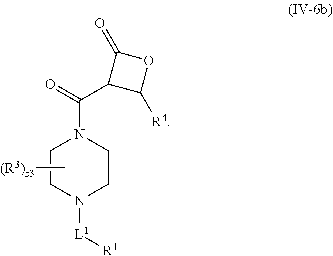

R¹, L¹, R³, z3, and R⁴ are as described herein, including in embodiments.

In embodiments, the compound has the formula:

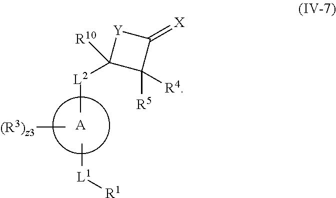

Ring A, X, Y, R¹, L¹, L², R³, z3, R⁴, R⁵, and R¹⁰ are as described herein, including in embodiments.

In embodiments, the compound has the formula:

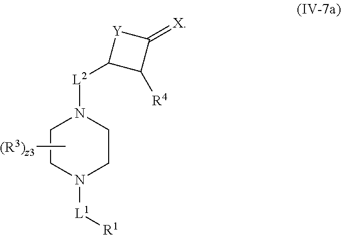

X, Y, R¹, L¹, L², R³, z3, and R⁴ are as described herein, including in embodiments.

In embodiments, the compound has the formula:

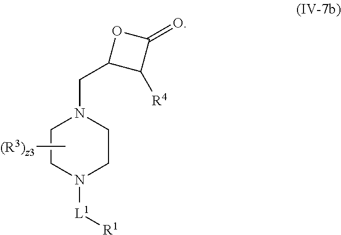

R¹, L¹, R³, z3, and R⁴ are as described herein, including in embodiments.

In embodiments, the compound has the formula:

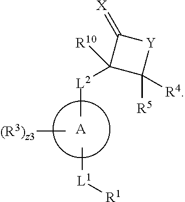

Ring A, X, Y, R¹, L¹, L², R³, z3, R⁴, R⁵, and R¹⁰ are as described herein, including in embodiments.

In embodiments, the compound has the formula:

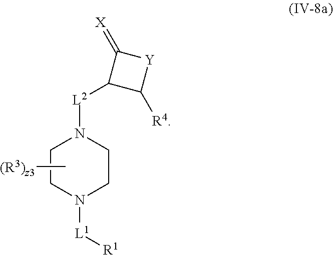

X, Y, R¹, L¹, L², R³, z3, and R⁴ are as described herein, including in embodiments.

In embodiments, the compound has the formula:

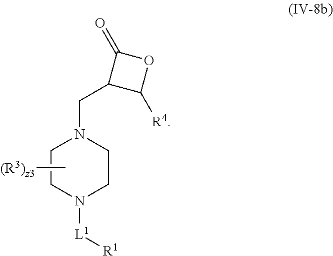

R¹, L¹, R³, z3, and R⁴ are as described herein, including in embodiments.

In embodiments, the compound has the formula:

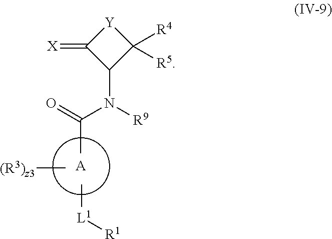

Ring A, X, Y, R¹, L¹, R³, z3, R⁴, R⁵, and R⁹ are as described herein, including in embodiments.

In embodiments, the compound has the formula:

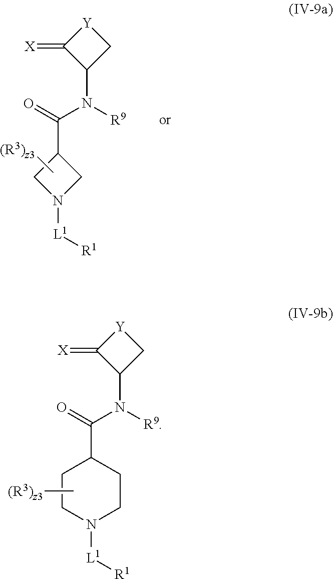

X, Y, R¹, L¹, R³, z3, and R⁹ are as described herein, including in embodiments. In embodiments, the compound has the formula:

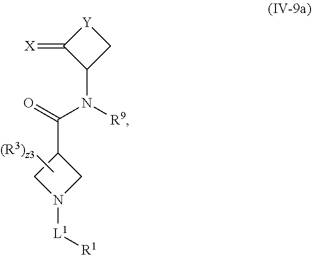

wherein X, Y, R¹, L¹, R³, z3, and R⁹ are as described herein, including in embodiments. In embodiments, the compound has the formula:

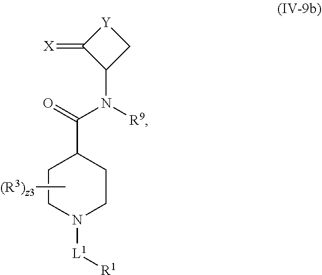

wherein X, Y, R¹, L¹, R³, z3, and R⁹ are as described herein, including in embodiments.

In embodiments, the compound has the formula:

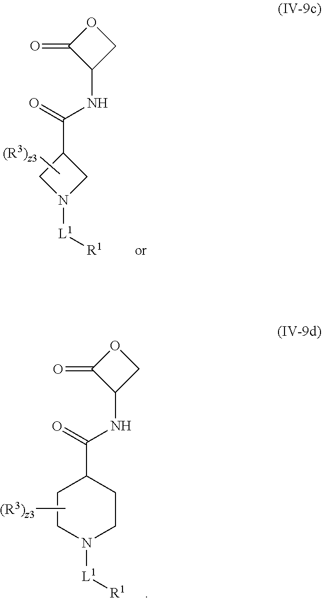

R¹, L¹, R³, and z3 are as described herein, including in embodiments. In embodiments, the compound has the formula:

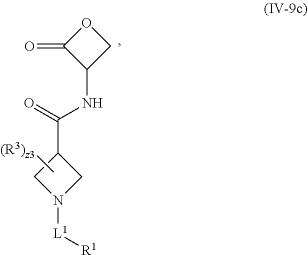

wherein R¹, L¹, R³, and z3 are as described herein, including in embodiments. In embodiments, the compound has the formula:

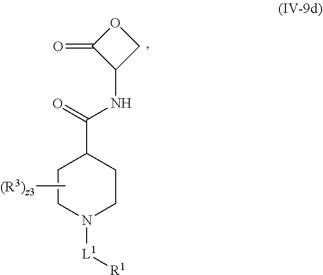

wherein R¹, L¹, R³, and z3 are as described herein, including in embodiments.

In embodiments, the compound has the formula:

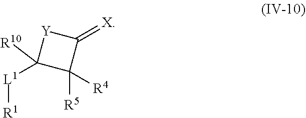

X, Y, R¹, L¹, R⁴, R⁵, and R¹⁰ are as described herein, including in embodiments.

In embodiments, the compound has the formula:

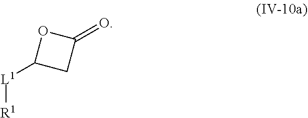

R¹ and L¹ are as described herein, including in embodiments.

In embodiments, the compound has the formula:

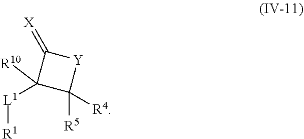

X, Y, R¹, L¹, R⁴, R⁵, and R¹⁰ are as described herein, including in embodiments.

In embodiments, the compound has the formula:

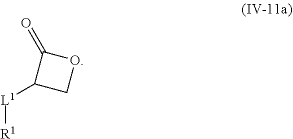

R¹ and L¹ are as described herein, including in embodiments.

In embodiments, a substituted R² (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R² is substituted with a plurality of groups selected from substituent groups, size-limited substituent groups, and lower substituent groups; each substituent group, size-limited substituent group, and/or lower substituent group may optionally be different. In embodiments, when R² is substituted, it is substituted with at least one substituent group. In embodiments, when R² is substituted, it is substituted with at least one size-limited substituent group. In embodiments, when R² is substituted, it is substituted with at least one lower substituent group.

In embodiments, R² is hydrogen, —CF₃, —CH₂F, —CHF₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R² is hydrogen or unsubstituted C₁-C₄ alkyl. In embodiments, R² is hydrogen. In embodiments, R² is unsubstituted C₁-C₄ alkyl. In embodiments, R² is unsubstituted methyl. In embodiments, R² is unsubstituted ethyl. In embodiments, R² is unsubstituted propyl. In embodiments, R² is unsubstituted n-propyl. In embodiments, R² is unsubstituted isopropyl. In embodiments, R² is unsubstituted butyl. In embodiments, R² is unsubstituted n-butyl. In embodiments, R² is unsubstituted isobutyl. In embodiments, R² is unsubstituted tert-butyl.

In embodiments, a substituted R³ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R³ is substituted with a plurality of groups selected from substituent groups, size-limited substituent groups, and lower substituent groups; each substituent group, size-limited substituent group, and/or lower substituent group may optionally be different. In embodiments, when R³ is substituted, it is substituted with at least one substituent group. In embodiments, when R³ is substituted, it is substituted with at least one size-limited substituent group. In embodiments, when R³ is substituted, it is substituted with at least one lower substituent group.

In embodiments, a substituted ring formed when two R³ substituents are joined (e.g., substituted cycloalkyl, substituted heterocycloalkyl, substituted aryl, and/or substituted heteroaryl) is substituted with at least one substituent group, size-limited substituent group, or lower substituent group; wherein if the substituted ring formed when two R³ substituents are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when two R³ substituents are joined is substituted, it is substituted with at least one substituent group. In embodiments, when the substituted ring formed when two R³ substituents are joined is substituted, it is substituted with at least one size-limited substituent group. In embodiments, when the substituted ring formed when two R³ substituents are joined is substituted, it is substituted with at least one lower substituent group.

In embodiments, R³ is independently halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); two R³ substituents may optionally be joined to form a substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, R³ is independently oxo. In embodiments, R³ is independently halogen. In embodiments, R³ is independently —F. In embodiments, R³ is independently —Cl. In embodiments, R³ is independently —Br. In embodiments, R³ is independently —I. In embodiments, R³ is independently —CCl₃. In embodiments, R³ is independently —CBr₃. In embodiments, R³ is independently —CF₃. In embodiments, R³ is independently —CI₃. In embodiments, R³ is independently —CH₂Cl. In embodiments, R³ is independently —CH₂Br. In embodiments, R³ is independently —CH₂F. In embodiments, R³ is independently —CH₂I. In embodiments, R³ is independently —CHCl₂. In embodiments, R³ is independently —CHBr₂. In embodiments, R³ is independently —CHF₂. In embodiments, R³ is independently —CHI₂. In embodiments, R³ is independently —CN. In embodiments, R³ is independently —OH. In embodiments, R³ is independently —NH₂. In embodiments, R³ is independently —COOH. In embodiments, R³ is independently —CONH₂. In embodiments, R³ is independently —NO₂. In embodiments, R³ is independently —SH. In embodiments, R³ is independently —SO₃H. In embodiments, R³ is independently —OSO₃H. In embodiments, R³ is independently —SO₂NH₂. In embodiments, R³ is independently —NHNH₂. In embodiments, R³ is independently —ONH₂. In embodiments, R³ is independently —NHC(O)NHNH₂. In embodiments, R³ is independently —NHC(O)NH₂. In embodiments, R³ is independently —NHSO₂H. In embodiments, R³ is independently —NHC(O)H. In embodiments, R³ is independently —NHC(O)OH. In embodiments, R³ is independently —NHOH. In embodiments, R³ is independently —OCCl₃. In embodiments, R³ is independently —OCBr₃. In embodiments, R³ is independently —OCF₃. In embodiments, R³ is independently —OCI₃. In embodiments, R³ is independently —OCH₂Cl. In embodiments, R³ is independently —OCH₂Br. In embodiments, R³ is independently —OCH₂F. In embodiments, R³ is independently —OCH₂I. In embodiments, R³ is independently —OCHCl₂. In embodiments, R³ is independently —OCHBr₂. In embodiments, R³ is independently —OCHF₂. In embodiments, R³ is independently —OCHI₂. In embodiments, R³ is independently unsubstituted C₁-C₄ alkyl. In embodiments, R³ is independently unsubstituted methyl. In embodiments, R³ is independently unsubstituted ethyl. In embodiments, R³ is independently unsubstituted propyl. In embodiments, R³ is independently unsubstituted n-propyl. In embodiments, R³ is independently unsubstituted isopropyl. In embodiments, R³ is independently unsubstituted butyl. In embodiments, R³ is independently unsubstituted n-butyl. In embodiments, R³ is independently unsubstituted isobutyl. In embodiments, R³ is independently unsubstituted tert-butyl. In embodiments, R³ is independently unsubstituted 2 to 4 membered heteroalkyl. In embodiments, R³ is independently unsubstituted methoxy. In embodiments, R³ is independently unsubstituted ethoxy. In embodiments, R³ is independently unsubstituted propoxy. In embodiments, R³ is independently unsubstituted n-propoxy. In embodiments, R³ is independently unsubstituted isopropoxy. In embodiments, R³ is independently unsubstituted butoxy. In embodiments, R³ is independently unsubstituted n-butoxy. In embodiments, R³ is independently unsubstituted isobutoxy. In embodiments, R³ is independently unsubstituted tert-butoxy.

In embodiments, two R³ substituents are joined to form a substituted or unsubstituted cycloalkyl or substituted or unsubstituted heterocycloalkyl. In embodiments, two R³ substituents are joined to form a substituted or unsubstituted C₃-C₈ cycloalkyl. In embodiments, two R³ substituents are joined to form a substituted or unsubstituted 3 to 8 membered heterocycloalkyl.

In embodiments, z3 is 0. In embodiments, z3 is 1. In embodiments, z3 is 2. In embodiments, z3 is 3. In embodiments, z3 is 4. In embodiments, z3 is 5. In embodiments, z3 is 6. In embodiments, z3 is 7. In embodiments, z3 is 8. In embodiments, z3 is 9. In embodiments, z3 is 10.

In embodiments, a substituted R⁴ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁴ is substituted with a plurality of groups selected from substituent groups, size-limited substituent groups, and lower substituent groups; each substituent group, size-limited substituent group, and/or lower substituent group may optionally be different. In embodiments, when R⁴ is substituted, it is substituted with at least one substituent group. In embodiments, when R⁴ is substituted, it is substituted with at least one size-limited substituent group. In embodiments, when R⁴ is substituted, it is substituted with at least one lower substituent group.

In embodiments, R⁴ is hydrogen. In embodiments, R⁴ is halogen. In embodiments, R⁴ is —F. In embodiments, R⁴ is —Cl. In embodiments, R⁴ is —Br. In embodiments, R⁴ is —I. In embodiments, R⁴ is —CCl₃. In embodiments, R⁴ is —CBr₃. In embodiments, R⁴ is —CF₃. In embodiments, R⁴ is —CI₃. In embodiments, R⁴ is —CH₂Cl. In embodiments, R⁴ is —CH₂Br. In embodiments, R⁴ is —CH₂F. In embodiments, R⁴ is —CH₂I. In embodiments, R⁴ is —CHCl₂. In embodiments, R⁴ is —CHBr₂. In embodiments, R⁴ is —CHF₂. In embodiments, R⁴ is —CHI₂. In embodiments, R⁴ is —CN. In embodiments, R⁴ is —OH. In embodiments, R⁴ is —NH₂. In embodiments, R⁴ is —COOH. In embodiments, R⁴ is —CONH₂. In embodiments, R⁴ is —NO₂. In embodiments, R⁴ is —SH. In embodiments, R⁴ is —SO₃H. In embodiments, R⁴ is —OSO₃H. In embodiments, R⁴ is —SO₂NH₂. In embodiments, R⁴ is —NHNH₂. In embodiments, R⁴ is —ONH₂. In embodiments, R⁴ is —NHC(O)NHNH₂. In embodiments, R⁴ is —NHC(O)NH₂. In embodiments, R⁴ is —NHSO₂H. In embodiments, R⁴ is —NHC(O)H. In embodiments, R⁴ is —NHC(O)OH. In embodiments, R⁴ is-NHOH. In embodiments, R⁴ is —OCCl₃. In embodiments, R⁴ is —OCBr₃. In embodiments, R⁴ is —OCF₃. In embodiments, R⁴ is —OCI₃. In embodiments, R⁴ is —OCH₂Cl. In embodiments, R⁴ is —OCH₂Br. In embodiments, R⁴ is —OCH₂F. In embodiments, R⁴ is —OCH₂I. In embodiments, R⁴ is —OCHCl₂. In embodiments, R⁴ is —OCHBr₂. In embodiments, R⁴ is —OCHF₂. In embodiments, R⁴ is —OCHI₂. In embodiments, R⁴ is unsubstituted C₁-C₄ alkyl. In embodiments, R⁴ is unsubstituted methyl. In embodiments, R⁴ is unsubstituted ethyl. In embodiments, R⁴ is unsubstituted propyl. In embodiments, R⁴ is unsubstituted n-propyl. In embodiments, R⁴ is unsubstituted isopropyl. In embodiments, R⁴ is unsubstituted butyl. In embodiments, R⁴ is unsubstituted n-butyl. In embodiments, R⁴ is unsubstituted isobutyl. In embodiments, R⁴ is unsubstituted tert-butyl. In embodiments, R⁴ is unsubstituted 2 to 4 membered heteroalkyl. In embodiments, R⁴ is unsubstituted methoxy. In embodiments, R⁴ is unsubstituted ethoxy. In embodiments, R⁴ is unsubstituted propoxy. In embodiments, R⁴ is unsubstituted n-propoxy. In embodiments, R⁴ is unsubstituted isopropoxy. In embodiments, R⁴ is unsubstituted butoxy. In embodiments, R⁴ is unsubstituted n-butoxy. In embodiments, R⁴ is unsubstituted isobutoxy. In embodiments, R⁴ is unsubstituted tert-butoxy.

In embodiments, a substituted R⁵ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁵ is substituted with a plurality of groups selected from substituent groups, size-limited substituent groups, and lower substituent groups; each substituent group, size-limited substituent group, and/or lower substituent group may optionally be different. In embodiments, when R⁵ is substituted, it is substituted with at least one substituent group. In embodiments, when R⁵ is substituted, it is substituted with at least one size-limited substituent group. In embodiments, when R⁵ is substituted, it is substituted with at least one lower substituent group.

In embodiments, R⁵ is hydrogen. In embodiments, R⁵ is halogen. In embodiments, R⁵ is —F. In embodiments, R⁵ is —Cl. In embodiments, R⁵ is —Br. In embodiments, R⁵ is —I. In embodiments, R⁵ is —CCl₃. In embodiments, R⁵ is —CBr₃. In embodiments, R⁵ is —CF₃. In embodiments, R⁵ is —CI₃. In embodiments, R⁵ is —CH₂Cl. In embodiments, R⁵ is —CH₂Br. In embodiments, R⁵ is —CH₂F. In embodiments, R⁵ is —CH₂I. In embodiments, R⁵ is —CHCl₂. In embodiments, R⁵ is —CHBr₂. In embodiments, R⁵ is —CHF₂. In embodiments, R⁵ is —CHI₂. In embodiments, R⁵ is —CN. In embodiments, R⁵ is —OH. In embodiments, R⁵ is —NH₂. In embodiments, R⁵ is —COOH. In embodiments, R⁵ is —CONH₂. In embodiments, R⁵ is —NO₂. In embodiments, R⁵ is —SH. In embodiments, R⁵ is —SO₃H. In embodiments, R⁵ is —OSO₃H. In embodiments, R⁵ is —SO₂NH₂. In embodiments, R⁵ is —NHNH₂. In embodiments, R⁵ is —ONH₂. In embodiments, R⁵ is —NHC(O)NHNH₂. In embodiments, R⁵ is —NHC(O)NH₂. In embodiments, R⁵ is —NHSO₂H. In embodiments, R⁵ is —NHC(O)H. In embodiments, R⁵ is —NHC(O)OH. In embodiments, R⁵ is —NHOH. In embodiments, R⁵ is —OCCl₃. In embodiments, R⁵ is —OCBr₃. In embodiments, R⁵ is —OCF₃. In embodiments, R⁵ is —OCI₃. In embodiments, R⁵ is —OCH₂Cl. In embodiments, R⁵ is —OCH₂Br. In embodiments, R⁵ is —OCH₂F. In embodiments, R⁵ is —OCH₂I. In embodiments, R⁵ is —OCHCl₂. In embodiments, R⁵ is —OCHBr₂. In embodiments, R⁵ is —OCHF₂. In embodiments, R⁵ is —OCHI₂. In embodiments, R⁵ is unsubstituted C₁-C₄ alkyl. In embodiments, R⁵ is unsubstituted methyl. In embodiments, R⁵ is unsubstituted ethyl. In embodiments, R⁵ is unsubstituted propyl. In embodiments, R⁵ is unsubstituted n-propyl. In embodiments, R⁵ is unsubstituted isopropyl. In embodiments, R⁵ is unsubstituted butyl. In embodiments, R⁵ is unsubstituted n-butyl. In embodiments, R⁵ is unsubstituted isobutyl. In embodiments, R⁵ is unsubstituted tert-butyl. In embodiments, R⁵ is unsubstituted 2 to 4 membered heteroalkyl. In embodiments, R⁵ is unsubstituted methoxy. In embodiments, R⁵ is unsubstituted ethoxy. In embodiments, R⁵ is unsubstituted propoxy. In embodiments, R⁵ is unsubstituted n-propoxy. In embodiments, R⁵ is unsubstituted isopropoxy. In embodiments, R⁵ is unsubstituted butoxy. In embodiments, R⁵ is unsubstituted n-butoxy. In embodiments, R⁵ is unsubstituted isobutoxy. In embodiments, R⁵ is unsubstituted tert-butoxy.

In embodiments, R⁴ is hydrogen and R⁵ is not hydrogen. In embodiments, R⁵ is hydrogen, and R⁴ is not hydrogen.

In embodiments, R⁵ is hydrogen and R⁴ is substituted or unsubstituted C₁-C₄ alkyl. In embodiments, R⁵ is hydrogen and R⁴ is unsubstituted C₁-C₄ alkyl. In embodiments, R⁵ is hydrogen and R⁴ is substituted or unsubstituted methyl or substituted or unsubstituted isopropyl. In embodiments, R⁴ is hydrogen and R⁵ is unsubstituted methyl. In embodiments, R⁵ is hydrogen, and R⁴ is unsubstituted methyl. In embodiments, R⁴ is hydrogen and R⁵ is unsubstituted isopropyl. In embodiments, R⁵ is hydrogen, and R⁴ is unsubstituted isopropyl. In embodiments, R⁴ and R⁵ are independently substituted or unsubstituted C₁-C₃ alkyl. In embodiments, R⁴ and R⁵ are independently unsubstituted C₁-C₃ alkyl. In embodiments, R⁴ and R⁵ are unsubstituted methyl.

In embodiments, a substituted R⁹ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁹ is substituted with a plurality of groups selected from substituent groups, size-limited substituent groups, and lower substituent groups; each substituent group, size-limited substituent group, and/or lower substituent group may optionally be different. In embodiments, when R⁹ is substituted, it is substituted with at least one substituent group. In embodiments, when R⁹ is substituted, it is substituted with at least one size-limited substituent group. In embodiments, when R⁹ is substituted, it is substituted with at least one lower substituent group.

In embodiments, R⁹ is hydrogen, —CF₃, —CH₂F, —CHF₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R⁹ is hydrogen or unsubstituted C₁-C₄ alkyl. In embodiments, R⁹ is hydrogen. In embodiments, R⁹ is unsubstituted C₁-C₄ alkyl. In embodiments, R⁹ is unsubstituted methyl. In embodiments, R⁹ is unsubstituted ethyl. In embodiments, R⁹ is unsubstituted propyl. In embodiments, R⁹ is unsubstituted n-propyl. In embodiments, R⁹ is unsubstituted isopropyl. In embodiments, R⁹ is unsubstituted butyl. In embodiments, R⁹ is unsubstituted n-butyl. In embodiments, R⁹ is unsubstituted isobutyl. In embodiments, R⁹ is unsubstituted tert-butyl.

In embodiments, a substituted R¹⁰ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹⁰ is substituted with a plurality of groups selected from substituent groups, size-limited substituent groups, and lower substituent groups; each substituent group, size-limited substituent group, and/or lower substituent group may optionally be different. In embodiments, when R¹⁰ is substituted, it is substituted with at least one substituent group. In embodiments, when R¹⁰ is substituted, it is substituted with at least one size-limited substituent group. In embodiments, when R¹⁰ is substituted, it is substituted with at least one lower substituent group.

In embodiments, R¹⁰ is hydrogen. In embodiments, R¹⁰ is halogen. In embodiments, R¹⁰ is —F. In embodiments, R¹⁰ is —Cl. In embodiments, R¹⁰ is —Br. In embodiments, R¹⁰ is —I. In embodiments, R¹⁰ is —CCl₃. In embodiments, R¹⁰ is —CBr₃. In embodiments, R¹⁰ is —CF₃. In embodiments, R¹⁰ is —CI₃. In embodiments, R¹⁰ is —CH₂Cl. In embodiments, R¹⁰ is —CH₂Br. In embodiments, R¹⁰ is —CH₂F. In embodiments, R¹⁰ is —CH₂I. In embodiments, R¹⁰ is —CHCl₂. In embodiments, R¹⁰ is —CHBr₂. In embodiments, R¹⁰ is —CHF₂. In embodiments, R¹⁰ is —CHI₂. In embodiments, R¹⁰ is —CN. In embodiments, R¹⁰ is —OH. In embodiments, R¹⁰ is —NH₂. In embodiments, R¹⁰ is —COOH. In embodiments, R¹⁰ is —CONH₂. In embodiments, R¹⁰ is —NO₂. In embodiments, R¹⁰ is —SH. In embodiments, R¹⁰ is —SO₃H. In embodiments, R¹⁰ is —OSO₃H. In embodiments, R¹⁰ is —SO₂NH₂. In embodiments, R¹⁰ is —NHNH₂. In embodiments, R¹⁰ is —ONH₂. In embodiments, R¹⁰ is —NHC(O)NHNH₂. In embodiments, R¹⁰ is —NHC(O)NH₂. In embodiments, R¹⁰ is —NHSO₂H. In embodiments, R¹⁰ is —NHC(O)H. In embodiments, R¹⁰ is —NHC(O)OH. In embodiments, R¹⁰ is —NHOH. In embodiments, R¹⁰ is —OCCl₃. In embodiments, R¹⁰ is —OCBr₃. In embodiments, R¹⁰ is —OCF₃. In embodiments, R¹⁰ is —OCI₃. In embodiments, R¹⁰ is —OCH₂Cl. In embodiments, R¹⁰ is —OCH₂Br. In embodiments, R¹⁰ is —OCH₂F. In embodiments, R¹⁰ is —OCH₂I. In embodiments, R¹⁰ is —OCHCl₂. In embodiments, R¹⁰ is —OCHBr₂. In embodiments, R¹⁰ is —OCHF₂. In embodiments, R¹⁰ is —OCHI₂. In embodiments, R¹⁰ is unsubstituted C₁-C₄ alkyl. In embodiments, R¹⁰ is unsubstituted methyl. In embodiments, R¹⁰ is unsubstituted ethyl. In embodiments, R¹⁰ is unsubstituted propyl. In embodiments, R¹⁰ is unsubstituted n-propyl. In embodiments, R¹⁰ is unsubstituted isopropyl. In embodiments, R¹⁰ is unsubstituted butyl. In embodiments, R¹⁰ is unsubstituted n-butyl. In embodiments, R¹⁰ is unsubstituted isobutyl. In embodiments, R¹⁰ is unsubstituted tert-butyl. In embodiments, R¹⁰ is unsubstituted 2 to 4 membered heteroalkyl. In embodiments, R¹⁰ is unsubstituted methoxy. In embodiments, R¹⁰ is unsubstituted ethoxy. In embodiments, R¹⁰ is unsubstituted propoxy. In embodiments, R¹⁰ is unsubstituted n-propoxy. In embodiments, R¹⁰ is unsubstituted isopropoxy. In embodiments, R¹⁰ is unsubstituted butoxy. In embodiments, R¹⁰ is unsubstituted n-butoxy. In embodiments, R¹⁰ is unsubstituted isobutoxy. In embodiments, R¹⁰ is unsubstituted tert-butoxy.

In embodiments, L² is unsubstituted methylene. In embodiments, L² is unsubstituted ethylene. In embodiments, L² is unsubstituted propylene. In embodiments, L² is unsubstituted n-propylene. In embodiments, L² is unsubstituted isopropylene. In embodiments, L² is unsubstituted butylene. In embodiments, L² is unsubstituted n-butylene. In embodiments, L² is unsubstituted isobutylene. In embodiments, L² is unsubstituted tert-butylene.

In embodiments, L¹ is -L¹⁰¹-L¹⁰²-L¹⁰³-.

L¹⁰¹ is connected directly to E¹ or E².

L¹⁰¹ is a bond, —C(O)—, —C(O)O—, —OC(O)—, —O—, —S—, —NR¹⁰¹—, —C(O)NR¹⁰¹—, —NR¹⁰¹C(O)—, —NR¹⁰¹C(O)O—, —OC(O)NR¹⁰¹—, —NR¹⁰¹C(O)NR¹⁰¹—, —NR¹⁰¹C(NH)NR¹⁰¹—, —S(O)₂—, —NR¹⁰¹S(O)₂—, —S(O)₂NR¹⁰¹—, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), or substituted or unsubstituted heteroarylene (e.g., 5 to 10 membered, 5 to 9 membered, or 5 to 6 membered).

L¹⁰² is a bond, —C(O)—, —C(O)O—, —OC(O)—, —O—, —S—, —NR¹⁰²—, —C(O)NR¹⁰²—, —NR¹⁰²C(O)—, —NR¹⁰²C(O)O—, —OC(O)NR¹⁰²—, —NR¹⁰²C(O)NR¹⁰²—, —NR¹⁰²C(NH)NR¹⁰²—, —S(O)₂—, —NR¹⁰²S(O)₂—, —S(O)₂NR¹⁰²—, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), or substituted or unsubstituted heteroarylene (e.g., 5 to 10 membered, 5 to 9 membered, or 5 to 6 membered).

L¹⁰³ is a bond, —C(O)—, —C(O)O—, —OC(O)—, —O—, —S—, —NR¹⁰³—, —C(O)NR¹⁰³—, —NR¹⁰³C(O)—, —NR¹⁰³C(O)O—, —OC(O)NR¹⁰³—, —NR¹⁰³C(O)NR¹⁰³—, —NR¹⁰³C(NH)NR¹⁰³—, —S(O)₂—, —NR¹⁰³S(O)₂—, —S(O)₂NR¹⁰³—, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), or substituted or unsubstituted heteroarylene (e.g., 5 to 10 membered, 5 to 9 membered, or 5 to 6 membered).

Each R¹⁰¹, R¹⁰², and R¹⁰³ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, a substituted L¹⁰¹ (e.g., substituted alkylene, substituted heteroalkylene, substituted cycloalkylene, substituted heterocycloalkylene, substituted arylene, and/or substituted heterarylene) is substituted with at least one substituent group, size-limited substituent group, or lower substituent group; wherein if the substituted L¹⁰¹ is substituted with a plurality of groups selected from substituent groups, size-limited substituent groups, and lower substituent groups; each substituent group, size-limited substituent group, and/or lower substituent group may optionally be different. In embodiments, when L¹⁰¹ is substituted, it is substituted with at least one substituent group. In embodiments, when L¹⁰¹ is substituted, it is substituted with at least one size-limited substituent group. In embodiments, when L¹⁰¹ is substituted, it is substituted with at least one lower substituent group.

In embodiments, L¹⁰¹ is a bond, —C(O)—, —C(O)O—, —OC(O)—, —O—, —S—, —NH—, —C(O)NH—, —NHC(O)—, —NHC(O)O—, —OC(O)NH—, —NHC(O)NH—, —NHC(NH)NH—, —S(O)₂—, —NHS(O)₂—, —S(O)₂NH—, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), or substituted or unsubstituted heteroarylene (e.g., 5 to 10 membered, 5 to 9 membered, or 5 to 6 membered).

In embodiments, L¹⁰¹ is a bond. In embodiments, L¹⁰¹ is —C(O)—. In embodiments, L¹⁰¹ is —C(O)O—. In embodiments, L¹⁰¹ is —OC(O)—. In embodiments, L¹⁰¹ is —O—. In embodiments, L¹⁰¹ is —S—. In embodiments, L¹⁰¹ is —NR¹⁰¹—. In embodiments, L¹⁰¹ is —NH—. In embodiments, L¹⁰¹ is —C(O)NR¹⁰¹—. In embodiments, L¹⁰¹ is —C(O)NH—. In embodiments, L¹⁰¹ is —NR¹⁰¹C(O)—. In embodiments, L¹⁰¹ is —NHC(O)—. In embodiments, L¹⁰¹ is —NR¹⁰¹C(O)O—. In embodiments, L¹⁰¹ is —NHC(O)O—. In embodiments, L¹⁰¹ is —OC(O)NR¹⁰¹—. In embodiments, L¹⁰¹ is —OC(O)NH—. In embodiments, L¹⁰¹ is —NR¹⁰¹C(O)NR¹⁰¹—. In embodiments, L¹⁰¹ is —NHC(O)NH—. In embodiments, L¹⁰¹ is —NR¹⁰¹C(NH)NR¹⁰¹—. In embodiments, L¹⁰¹ is —NHC(NH)NH—. In embodiments, L¹⁰¹ is —S(O)₂—. In embodiments, L¹⁰¹ is —NR¹⁰¹S(O)₂—. In embodiments, L¹⁰¹ is —NHS(O)₂—. In embodiments, L¹⁰¹ is —S(O)₂NR¹⁰¹—. In embodiments, L¹⁰¹ is —S(O)₂NH—. In embodiments, L¹⁰¹ is substituted (e.g., oxo-substituted) or unsubstituted C₁-C₆ alkylene. In embodiments, L¹⁰¹ is substituted (e.g., oxo-substituted) or unsubstituted methylene. In embodiments, L¹⁰¹ is substituted (e.g., oxo-substituted) or unsubstituted ethylene. In embodiments, L¹⁰¹ is substituted (e.g., oxo-substituted) or unsubstituted propylene. In embodiments, L¹⁰¹ is substituted (e.g., oxo-substituted) or unsubstituted n-propylene. In embodiments, L¹⁰¹ is substituted (e.g., oxo-substituted) or unsubstituted isopropylene. In embodiments, L¹⁰¹ is substituted (e.g., oxo-substituted) or unsubstituted butylene. In embodiments, L¹⁰¹ is substituted (e.g., oxo-substituted) or unsubstituted n-butylene. In embodiments, L¹⁰¹ is substituted (e.g., oxo-substituted) or unsubstituted isobutylene. In embodiments, L¹⁰¹ is substituted (e.g., oxo-substituted) or unsubstituted tert-butylene. In embodiments, L¹⁰¹ is substituted (e.g., oxo-substituted) or unsubstituted pentylene. In embodiments, L¹⁰¹ is substituted (e.g., oxo-substituted) or unsubstituted hexylene. In embodiments, L¹⁰¹ is substituted (e.g., oxo-substituted) or unsubstituted 2 to 6 membered heteroalkylene. In embodiments, L¹⁰¹ is

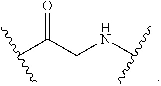 [SMILES: *NCC(*)=O]

In embodiments, a substituted R¹⁰¹ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹⁰¹ is substituted with a plurality of groups selected from substituent groups, size-limited substituent groups, and lower substituent groups; each substituent group, size-limited substituent group, and/or lower substituent group may optionally be different. In embodiments, when R¹⁰¹ is substituted, it is substituted with at least one substituent group. In embodiments, when R¹⁰¹ is substituted, it is substituted with at least one size-limited substituent group. In embodiments, when R¹⁰¹ is substituted, it is substituted with at least one lower substituent group.

In embodiments, R¹⁰¹ is independently hydrogen or unsubstituted C₁-C₄ alkyl. In embodiments, R¹⁰¹ is independently hydrogen. In embodiments, R¹⁰¹ is independently unsubstituted C₁-C₄ alkyl. In embodiments, R¹⁰¹ is independently unsubstituted methyl. In embodiments, R¹⁰¹ is independently unsubstituted ethyl. In embodiments, R¹⁰¹ is independently unsubstituted propyl. In embodiments, R¹⁰¹ is independently unsubstituted n-propyl. In embodiments, R¹⁰¹ is independently unsubstituted isopropyl. In embodiments, R¹⁰¹ is independently unsubstituted butyl. In embodiments, R¹⁰¹ is independently unsubstituted n-butyl. In embodiments, R¹⁰¹ is independently unsubstituted isobutyl. In embodiments, R¹⁰¹ is independently unsubstituted tert-butyl.

In embodiments, a substituted L¹⁰² (e.g., substituted alkylene, substituted heteroalkylene, substituted cycloalkylene, substituted heterocycloalkylene, substituted arylene, and/or substituted heterarylene) is substituted with at least one substituent group, size-limited substituent group, or lower substituent group; wherein if the substituted L¹⁰² is substituted with a plurality of groups selected from substituent groups, size-limited substituent groups, and lower substituent groups; each substituent group, size-limited substituent group, and/or lower substituent group may optionally be different. In embodiments, when L¹⁰² is substituted, it is substituted with at least one substituent group. In embodiments, when L¹⁰² is substituted, it is substituted with at least one size-limited substituent group. In embodiments, when L¹⁰² is substituted, it is substituted with at least one lower substituent group.

In embodiments, L¹⁰² is a bond, —C(O)—, —C(O)O—, —OC(O)—, —O—, —S—, —NH—, —C(O)NH—, —NHC(O)—, —NHC(O)O—, —OC(O)NH—, —NHC(O)NH—, —NHC(NH)NH—, —S(O)₂—, —NHS(O)₂—, —S(O)₂NH—, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), or substituted or unsubstituted heteroarylene (e.g., 5 to 10 membered, 5 to 9 membered, or 5 to 6 membered).

In embodiments, L¹⁰² is a bond. In embodiments, L¹⁰² is —C(O)—. In embodiments, L¹⁰² is —C(O)O—. In embodiments, L¹⁰² is —OC(O)—. In embodiments, L¹⁰² is —O—. In embodiments, L¹⁰² is —S—. In embodiments, L¹⁰² is —NR¹⁰²—. In embodiments, L¹⁰² is —NH—. In embodiments, L¹⁰² is —C(O)NR¹⁰²—. In embodiments, L¹⁰² is —C(O)NH—. In embodiments, L¹⁰² is —NR¹⁰²C(O)—. In embodiments, L¹⁰² is —NHC(O)—. In embodiments, L¹⁰² is —NR¹⁰²C(O)O—. In embodiments, L¹⁰² is —NHC(O)O—. In embodiments, L¹⁰² is —OC(O)NR¹⁰²—. In embodiments, L¹⁰² is —OC(O)NH—. In embodiments, L¹⁰² is —NR¹⁰²C(O)NR¹⁰²—. In embodiments, L¹⁰² is —NHC(O)NH—. In embodiments, L¹⁰² is —NR¹⁰²C(NH)NR¹⁰²—. In embodiments, L¹⁰² is —NHC(NH)NH—. In embodiments, L¹⁰² is —S(O)₂—. In embodiments, L¹⁰² is —NR¹⁰²S(O)₂—. In embodiments, L¹⁰² is —NHS(O)₂—. In embodiments, L¹⁰² is —S(O)₂NR¹⁰²—. In embodiments, L¹⁰² is —S(O)₂NH—. In embodiments, L¹⁰² is substituted (e.g., oxo-substituted) or unsubstituted C₁-C₆ alkylene. In embodiments, L¹⁰² is substituted (e.g., oxo-substituted) or unsubstituted methylene. In embodiments, L¹⁰² is substituted (e.g., oxo-substituted) or unsubstituted ethylene. In embodiments, L¹⁰² is substituted (e.g., oxo-substituted) or unsubstituted propylene. In embodiments, L¹⁰² is substituted (e.g., oxo-substituted) or unsubstituted n-propylene. In embodiments, L¹⁰² is substituted (e.g., oxo-substituted) or unsubstituted isopropylene. In embodiments, L¹⁰² is substituted (e.g., oxo-substituted) or unsubstituted butylene. In embodiments, L¹⁰² is substituted (e.g., oxo-substituted) or unsubstituted n-butylene. In embodiments, L¹⁰² is substituted (e.g., oxo-substituted) or unsubstituted isobutylene. In embodiments, L¹⁰² is substituted (e.g., oxo-substituted) or unsubstituted tert-butylene. In embodiments, L¹⁰² is substituted (e.g., oxo-substituted) or unsubstituted pentylene. In embodiments, L¹⁰² is substituted (e.g., oxo-substituted) or unsubstituted hexylene. In embodiments, L¹⁰² is substituted (e.g., oxo-substituted) or unsubstituted 2 to 6 membered heteroalkylene. In embodiments, L¹⁰² is

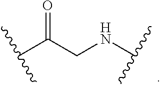 [SMILES: *NCC(*)=O]

In embodiments, a substituted R¹⁰² (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹⁰² is substituted with a plurality of groups selected from substituent groups, size-limited substituent groups, and lower substituent groups; each substituent group, size-limited substituent group, and/or lower substituent group may optionally be different. In embodiments, when R¹⁰² is substituted, it is substituted with at least one substituent group. In embodiments, when R¹⁰² is substituted, it is substituted with at least one size-limited substituent group. In embodiments, when R¹⁰² is substituted, it is substituted with at least one lower substituent group.

In embodiments, R¹⁰² is independently hydrogen or unsubstituted C₁-C₄ alkyl. In embodiments, R¹⁰² is independently hydrogen. In embodiments, R¹⁰² is independently unsubstituted C₁-C₄ alkyl. In embodiments, R¹⁰² is independently unsubstituted methyl. In embodiments, R¹⁰² is independently unsubstituted ethyl. In embodiments, R¹⁰² is independently unsubstituted propyl. In embodiments, R¹⁰² is independently unsubstituted n-propyl. In embodiments, R¹⁰² is independently unsubstituted isopropyl. In embodiments, R¹⁰² is independently unsubstituted butyl. In embodiments, R¹⁰² is independently unsubstituted n-butyl. In embodiments, R¹⁰² is independently unsubstituted isobutyl. In embodiments, R¹⁰² is independently unsubstituted tert-butyl.

In embodiments, a substituted L¹⁰³ (e.g., substituted alkylene, substituted heteroalkylene, substituted cycloalkylene, substituted heterocycloalkylene, substituted arylene, and/or substituted heterarylene) is substituted with at least one substituent group, size-limited substituent group, or lower substituent group; wherein if the substituted L¹⁰³ is substituted with a plurality of groups selected from substituent groups, size-limited substituent groups, and lower substituent groups; each substituent group, size-limited substituent group, and/or lower substituent group may optionally be different. In embodiments, when L¹⁰³ is substituted, it is substituted with at least one substituent group. In embodiments, when L¹⁰³ is substituted, it is substituted with at least one size-limited substituent group. In embodiments, when L¹⁰³ is substituted, it is substituted with at least one lower substituent group.

In embodiments, L¹⁰³ is a bond. In embodiments, L¹⁰³ is —C(O)—. In embodiments, L¹⁰³ is —C(O)O—. In embodiments, L¹⁰³ is —OC(O)—. In embodiments, L¹⁰³ is —O—. In embodiments, L¹⁰³ is —S—. In embodiments, L¹⁰³ is —NR¹⁰³—. In embodiments, L¹⁰³ is —NH—. In embodiments, L¹⁰³ is —C(O)NR¹⁰³—. In embodiments, L¹⁰³ is —C(O)NH—. In embodiments, L¹⁰³ is —NR¹⁰³C(O)—. In embodiments, L¹⁰³ is —NHC(O)—. In embodiments, L¹⁰³ is —NR¹⁰³C(O)O—. In embodiments, L¹⁰³ is —NHC(O)O—. In embodiments, L¹⁰³ is —OC(O)NR¹⁰³—. In embodiments, L¹⁰³ is —OC(O)NH—. In embodiments, L¹⁰³ is —NR¹⁰³C(O)NR¹⁰³—. In embodiments, L¹⁰³ is —NHC(O)NH—. In embodiments, L¹⁰³ is —NR¹⁰³C(NH)NR¹⁰³—. In embodiments, L¹⁰³ is —NHC(NH)NH—. In embodiments, L¹⁰³ is —S(O)₂—. In embodiments, L¹⁰³ is —NR¹⁰³S(O)₂—. In embodiments, L¹⁰³ is —NHS(O)₂—. In embodiments, L¹⁰³ is —S(O)₂NR¹⁰³—. In embodiments, L¹⁰³ is —S(O)₂NH—. In embodiments, L¹⁰³ is substituted (e.g., oxo-substituted) or unsubstituted C₁-C₆ alkylene. In embodiments, L¹⁰³ is substituted (e.g., oxo-substituted) or unsubstituted methylene. In embodiments, L¹⁰³ is substituted (e.g., oxo-substituted) or unsubstituted ethylene. In embodiments, L¹⁰³ is substituted (e.g., oxo-substituted) or unsubstituted propylene. In embodiments, L¹⁰³ is substituted (e.g., oxo-substituted) or unsubstituted n-propylene. In embodiments, L¹⁰³ is substituted (e.g., oxo-substituted) or unsubstituted isopropylene. In embodiments, L¹⁰³ is substituted (e.g., oxo-substituted) or unsubstituted butylene. In embodiments, L¹⁰³ is substituted (e.g., oxo-substituted) or unsubstituted n-butylene. In embodiments, L¹⁰³ is substituted (e.g., oxo-substituted) or unsubstituted isobutylene. In embodiments, L¹⁰³ is substituted (e.g., oxo-substituted) or unsubstituted tert-butylene. In embodiments, L¹⁰³ is substituted (e.g., oxo-substituted) or unsubstituted pentylene. In embodiments, L¹⁰³ is substituted (e.g., oxo-substituted) or unsubstituted hexylene. In embodiments, L¹⁰³ is substituted (e.g., oxo-substituted) or unsubstituted 2 to 6 membered heteroalkylene. In embodiments, L¹⁰³ is

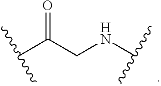 [SMILES: *NCC(*)=O]

In embodiments, a substituted R¹⁰³ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹⁰³ is substituted with a plurality of groups selected from substituent groups, size-limited substituent groups, and lower substituent groups; each substituent group, size-limited substituent group, and/or lower substituent group may optionally be different. In embodiments, when R¹⁰³ is substituted, it is substituted with at least one substituent group. In embodiments, when R¹⁰³ is substituted, it is substituted with at least one size-limited substituent group. In embodiments, when R¹⁰³ is substituted, it is substituted with at least one lower substituent group.

In embodiments, R¹⁰³ is independently hydrogen or unsubstituted C₁-C₄ alkyl. In embodiments, R¹⁰³ is independently hydrogen. In embodiments, R¹⁰³ is independently unsubstituted C₁-C₄ alkyl. In embodiments, R¹⁰³ is independently unsubstituted methyl. In embodiments, R¹⁰³ is independently unsubstituted ethyl. In embodiments, R¹⁰³ is independently unsubstituted propyl. In embodiments, R¹⁰³ is independently unsubstituted n-propyl. In embodiments, R¹⁰³ is independently unsubstituted isopropyl. In embodiments, R¹⁰³ is independently unsubstituted butyl. In embodiments, R¹⁰³ is independently unsubstituted n-butyl. In embodiments, R¹⁰³ is independently unsubstituted isobutyl. In embodiments, R¹⁰³ is independently unsubstituted tert-butyl.

In embodiments, L¹ is a bond. In embodiments, L¹ is —C(O)—. In embodiments, L¹ is a substituted 2 to 6 membered heteroalkylene. In embodiments, L¹ is

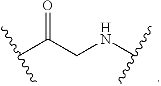 [SMILES: *NCC(*)=O]

In embodiments, the compound contacts a residue of K-Ras Switch II. In embodiments, the compound contacts a residue of H-Ras Switch II. In embodiments, the compound contacts a residue of N-Ras Switch II. In embodiments, wherein the compound contacts K-Ras (e.g., K-Ras(G12S), human K-Ras(G12S)), R¹ contacts V7, V9, G10, P34, T58, G60, Q61, E62, E63, R68, Y71, M72, Y96, Q99, or 1100. In embodiments, R¹ contacts at least one of G60, E62, or E63 of K-Ras (e.g., K-Ras(G12S), human K-Ras(G12S)). In embodiments, the compound does not contact the residues of K-Ras (e.g., K-Ras(G12S), human K-Ras(G12S)) that contact GTP. In embodiments, the compound does not contact the residues of K-Ras (e.g., K-Ras(G12S), human K-Ras(G12S)) that contact the guanine of GTP or GDP. In embodiments, the compound does not contact the residues of K-Ras (e.g., K-Ras(G12S), human K-Ras(G12S)) that contact GDP. In embodiments, R¹ contacts residues that contact Switch II in the GTP bound form of K-Ras (e.g., K-Ras(G12S), human K-Ras(G12S)). In embodiments, R¹ contacts residues that contact Switch II in the GDP bound form of K-Ras (e.g., K-Ras(G12S), human K-Ras(G12S)).

In embodiments, the compound contacts a residue of K-Ras Switch II. In embodiments, the compound contacts a residue of H-Ras Switch II. In embodiments, the compound contacts a residue of N-Ras Switch II. In embodiments, wherein the compound contacts K-Ras (e.g., K-Ras(G12D), human K-Ras(G12D), K-Ras(G13D), human K-Ras(G13D), K-Ras(G12E), human K-Ras(G12E)), R¹ contacts V7, V9, G10, P34, T58, G60, Q61, E62, E63, R68, Y71, M72, Y96, Q99, or 1100. In embodiments, R¹ contacts at least one of G60, E62, or E63 of K-Ras (e.g., K-Ras(G12D), human K-Ras(G12D), K-Ras(G13D), human K-Ras(G13D), K-Ras(G12E), human K-Ras(G12E)). In embodiments, the compound does not contact the residues of K-Ras (e.g., K-Ras(G12D), human K-Ras(G12D), K-Ras(G13D), human K-Ras(G13D), K-Ras(G12E), human K-Ras(G12E)) that contact GTP. In embodiments, the compound does not contact the residues of K-Ras (e.g., K-Ras(G12D), human K-Ras(G12D), K-Ras(G13D), human K-Ras(G13D), K-Ras(G12E), human K-Ras(G12E)) that contact the guanine of GTP or GDP. In embodiments, the compound does not contact the residues of K-Ras (e.g., K-Ras(G12D), human K-Ras(G12D), K-Ras(G13D), human K-Ras(G13D), K-Ras(G12E), human K-Ras(G12E)) that contact GDP. In embodiments, R¹ contacts residues that contact Switch II in the GTP bound form of K-Ras (e.g., K-Ras(G12D), human K-Ras(G12D), K-Ras(G13D), human K-Ras(G13D), K-Ras(G12E), human K-Ras(G12E)). In embodiments, R¹ contacts residues that contact Switch II in the GDP bound form of K-Ras (e.g., K-Ras(G12D), human K-Ras(G12D), K-Ras(G13D), human K-Ras(G13D), K-Ras(G12E), human K-Ras(G12E)).

In embodiments, R¹ is -L²⁰-R²⁰.

L²⁰ is a bond, —C(O)—, —C(O)O—, —OC(O)—, —O—, —S—, —NR²⁰⁰—, —C(O)NR²⁰⁰—, —NR²⁰⁰C(O)—, —NR²⁰⁰C(O)O—, —OC(O)NR²⁰⁰—, —NR²⁰⁰C(O)NR²⁰⁰—, —NR²⁰⁰C(NH)NR²⁰⁰—, —S(O)₂—, —NR²⁰⁰S(O)₂—, —S(O)₂NR²⁰⁰—, substituted or unsubstituted alkylene (e.g., C₁-C₈, C₁—C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), or substituted or unsubstituted heteroarylene (e.g., 5 to 10 membered, 5 to 9 membered, or 5 to 6 membered).

R²⁰⁰ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R²⁰ is hydrogen, halogen, —CX²⁰³, —CHX²⁰², —CH₂X²⁰, —OCX²⁰³, —OCH₂X²⁰, —OCHX²⁰₂, —CN, —SOₙ₂₀R^{20D}, —SOᵥ₂₀NR^{20A}R^{20B}, —NR^{20C}NR^{20A}R^{20B}, —ONR^{20A}R^{20B}, —NHC(O)NR^{20C}NR^{20A}R²⁰, —NHC(O)NR^{20A}R²⁰, —N(O)ₘ₂₀, —NR^{20A}R²⁰, —C(O)R^{20C}, —C(O)OR^{20C}, —C(O)NR^{20A}R^{20B}, —OR^{20D}, —SR^{20D}, —NR^{20A}SO₂R^{20D}, —NR^{20A}C(O)R^{20C}, —NR^{20A}C(O)OR^{20C}, —NR^{20A}OR^{20C}, —SF₅, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R^{20A}, R^{20B}, R^{20C}, and R^{20D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); R^{20A} and R^{20B} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered).

X²⁰ is independently —F, —Cl, —Br, or —I.

The symbol n20 is an integer from 0 to 4.

The symbols m20 and v20 are independently 1 or 2.

In embodiments, a substituted L²⁰ (e.g., substituted alkylene, substituted heteroalkylene, substituted cycloalkylene, substituted heterocycloalkylene, substituted arylene, and/or substituted heterarylene) is substituted with at least one substituent group, size-limited substituent group, or lower substituent group; wherein if the substituted L²⁰ is substituted with a plurality of groups selected from substituent groups, size-limited substituent groups, and lower substituent groups; each substituent group, size-limited substituent group, and/or lower substituent group may optionally be different. In embodiments, when L²⁰ is substituted, it is substituted with at least one substituent group. In embodiments, when L²⁰ is substituted, it is substituted with at least one size-limited substituent group. In embodiments, when L²⁰ is substituted, it is substituted with at least one lower substituent group.

In embodiments, L²⁰ is a bond, —C(O)—, —C(O)O—, —OC(O)—, —O—, —S—, —NH—, —C(O)NH—, —NHC(O)—, —NHC(O)O—, —OC(O)NH—, —NHC(O)NH—, —NHC(NH)NH—, —S(O)₂—, —NHS(O)₂—, —S(O)₂NH—, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), or substituted or unsubstituted heteroarylene (e.g., 5 to 10 membered, 5 to 9 membered, or 5 to 6 membered).

In embodiments, L²⁰ is a bond. In embodiments, L²⁰ is —C(O)—. In embodiments, L²⁰ is —C(O)O—. In embodiments, L²⁰ is —OC(O)—. In embodiments, L²⁰ is —O—. In embodiments, L²⁰ is —S—. In embodiments, L²⁰ is —NR²⁰⁰—. In embodiments, L²⁰ is —NH—. In embodiments, L²⁰ is —C(O)NR²⁰⁰—. In embodiments, L²⁰ is —C(O)NH—. In embodiments, L²⁰ is —NR²⁰⁰C(O)—. In embodiments, L²⁰ is —NHC(O)—. In embodiments, L²⁰ is —NR²⁰⁰C(O)O—. In embodiments, L²⁰ is —NHC(O)O—. In embodiments, L²⁰ is —OC(O)NR²⁰⁰—. In embodiments, L²⁰ is —OC(O)NH—. In embodiments, L²⁰ is —NR²⁰⁰C(O)NR²⁰⁰—. In embodiments, L²⁰ is —NHC(O)NH—. In embodiments, L²⁰ is —NR²⁰⁰C(NH)NR²⁰⁰—. In embodiments, L²⁰ is —NHC(NH)NH—. In embodiments, L²⁰ is —S(O)₂—. In embodiments, L²⁰ is —NR²⁰⁰S(O)₂—. In embodiments, L²⁰ is —NHS(O)₂—. In embodiments, L²⁰ is —S(O)₂NR²⁰⁰—. In embodiments, L²⁰ is —S(O)₂NH—. In embodiments, L²⁰ is substituted (e.g., oxo-substituted) or unsubstituted C₁-C₆ alkylene. In embodiments, L²⁰ is substituted (e.g., oxo-substituted) or unsubstituted methylene. In embodiments, L²⁰ is substituted (e.g., oxo-substituted) or unsubstituted ethylene. In embodiments, L²⁰ is substituted (e.g., oxo-substituted) or unsubstituted propylene. In embodiments, L²⁰ is substituted (e.g., oxo-substituted) or unsubstituted n-propylene. In embodiments, L²⁰ is substituted (e.g., oxo-substituted) or unsubstituted isopropylene. In embodiments, L²⁰ is substituted (e.g., oxo-substituted) or unsubstituted butylene. In embodiments, L²⁰ is substituted (e.g., oxo-substituted) or unsubstituted n-butylene. In embodiments, L²⁰ is substituted (e.g., oxo-substituted) or unsubstituted isobutylene. In embodiments, L²⁰ is substituted (e.g., oxo-substituted) or unsubstituted tert-butylene. In embodiments, L²⁰ is substituted (e.g., oxo-substituted) or unsubstituted pentylene. In embodiments, L²⁰ is substituted (e.g., oxo-substituted) or unsubstituted hexylene. In embodiments, L²⁰ is substituted (e.g., oxo-substituted) or unsubstituted 2 to 6 membered heteroalkylene.

In embodiments, a substituted R²⁰⁰ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R²⁰⁰ is substituted with a plurality of groups selected from substituent groups, size-limited substituent groups, and lower substituent groups; each substituent group, size-limited substituent group, and/or lower substituent group may optionally be different. In embodiments, when R²⁰⁰ is substituted, it is substituted with at least one substituent group. In embodiments, when R²⁰⁰ is substituted, it is substituted with at least one size-limited substituent group. In embodiments, when R²⁰⁰ is substituted, it is substituted with at least one lower substituent group.

In embodiments, R²⁰⁰ is independently hydrogen or unsubstituted C₁-C₄ alkyl. In embodiments, R²⁰⁰ is independently hydrogen. In embodiments, R²⁰⁰ is independently unsubstituted C₁-C₄ alkyl. In embodiments, R²⁰⁰ is independently unsubstituted methyl. In embodiments, R²⁰⁰ is independently unsubstituted ethyl. In embodiments, R²⁰⁰ is independently unsubstituted propyl. In embodiments, R²⁰⁰ is independently unsubstituted n-propyl. In embodiments, R²⁰⁰ is independently unsubstituted isopropyl. In embodiments, R²⁰⁰ is independently unsubstituted butyl. In embodiments, R²⁰⁰ is independently unsubstituted n-butyl. In embodiments, R²⁰⁰ is independently unsubstituted isobutyl. In embodiments, R²⁰⁰ is independently unsubstituted tert-butyl.

In embodiments, a substituted R²⁰ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R²⁰ is substituted with a plurality of groups selected from substituent groups, size-limited substituent groups, and lower substituent groups; each substituent group, size-limited substituent group, and/or lower substituent group may optionally be different. In embodiments, when R²⁰ is substituted, it is substituted with at least one substituent group. In embodiments, when R²⁰ is substituted, it is substituted with at least one size-limited substituent group. In embodiments, when R²⁰ is substituted, it is substituted with at least one lower substituent group.

In embodiments, a substituted R^{20A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{20A} is substituted with a plurality of groups selected from substituent groups, size-limited substituent groups, and lower substituent groups; each substituent group, size-limited substituent group, and/or lower substituent group may optionally be different. In embodiments, when R^{20A} is substituted, it is substituted with at least one substituent group. In embodiments, when R^{20A} is substituted, it is substituted with at least one size-limited substituent group. In embodiments, when R^{20A} is substituted, it is substituted with at least one lower substituent group.

In embodiments, a substituted R^{20B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{20B} is substituted with a plurality of groups selected from substituent groups, size-limited substituent groups, and lower substituent groups; each substituent group, size-limited substituent group, and/or lower substituent group may optionally be different. In embodiments, when R^{20B} is substituted, it is substituted with at least one substituent group. In embodiments, when R^{20B} is substituted, it is substituted with at least one size-limited substituent group. In embodiments, when R^{20B} is substituted, it is substituted with at least one lower substituent group.

In embodiments, a substituted ring formed when R^{20A} and R^{20B} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{20A} and R^{20B} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R^{20A} and R^{20B} substituents bonded to the same nitrogen atom are joined is substituted, it is substituted with at least one substituent group. In embodiments, when the substituted ring formed when R^{20A} and R^{20B} substituents bonded to the same nitrogen atom are joined is substituted, it is substituted with at least one size-limited substituent group. In embodiments, when the substituted ring formed when R^{20A} and R^{20B} substituents bonded to the same nitrogen atom are joined is substituted, it is substituted with at least one lower substituent group.

In embodiments, a substituted R^{20C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{20C} is substituted with a plurality of groups selected from substituent groups, size-limited substituent groups, and lower substituent groups; each substituent group, size-limited substituent group, and/or lower substituent group may optionally be different. In embodiments, when R^{20C} is substituted, it is substituted with at least one substituent group. In embodiments, when R^{20C} is substituted, it is substituted with at least one size-limited substituent group. In embodiments, when R^{20C} is substituted, it is substituted with at least one lower substituent group.

In embodiments, a substituted R^{20D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{20D} is substituted with a plurality of groups selected from substituent groups, size-limited substituent groups, and lower substituent groups; each substituent group, size-limited substituent group, and/or lower substituent group may optionally be different. In embodiments, when R^{20D} is substituted, it is substituted with at least one substituent group. In embodiments, when R^{20D} is substituted, it is substituted with at least one size-limited substituent group. In embodiments, when R^{20D} is substituted, it is substituted with at least one lower substituent group.

In embodiments, R²⁰ is substituted or unsubstituted cycloalkyl, substituted or unsubstituted heterocycloalkyl, substituted or unsubstituted aryl, or substituted or unsubstituted heteroaryl. In embodiments, R²⁰ is substituted or unsubstituted C₃-C₈ cycloalkyl, substituted or unsubstituted 3 to 8 membered heterocycloalkyl, substituted or unsubstituted C₆-C₁₀ aryl, or substituted or unsubstituted 5 to 10 membered heteroaryl. In embodiments, R²⁰ is substituted C₃-C₈ cycloalkyl. In embodiments, R²⁰ is substituted 3 to 8 membered heterocycloalkyl. In embodiments, R²⁰ is substituted C₆-C₁₀ aryl. In embodiments, R²⁰ is substituted phenyl. In embodiments, R²⁰ is substituted 5 to 10 membered heteroaryl. In embodiments, R²⁰ is substituted pyrimidyl. In embodiments, R²⁰ is substituted tetrahydropyridopyrimidyl. In embodiments, R²⁰ is substituted pyridopyrimidyl. In embodiments, R²⁰ is substituted quinazolinyl. In embodiments, R²⁰ is substituted pyrazolyl. In embodiments, R²⁰ is substituted piperazinyl.

In embodiments, R¹ is

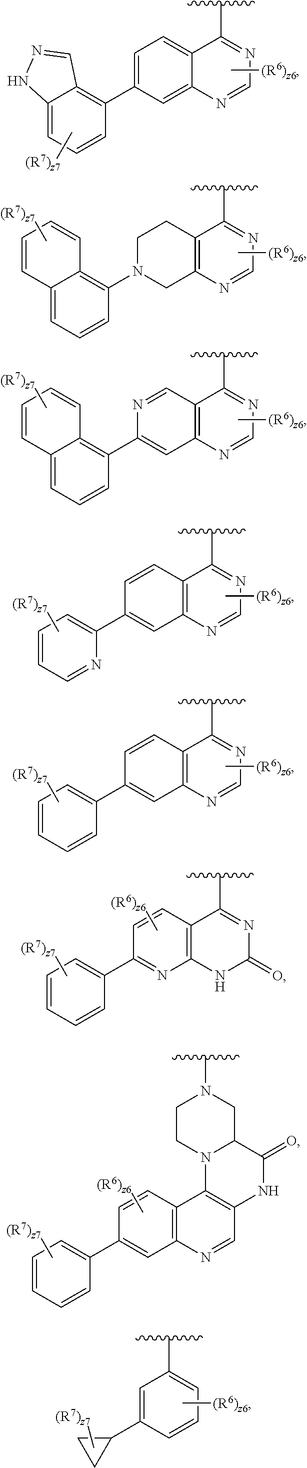

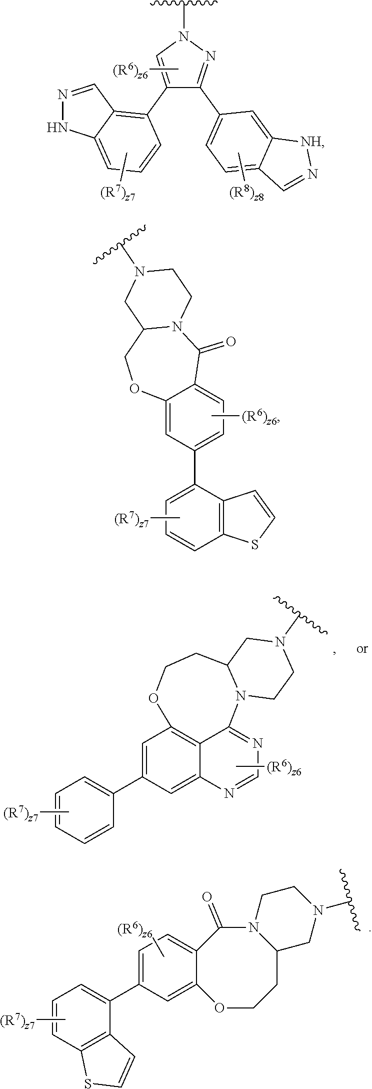

In embodiments, R¹ is

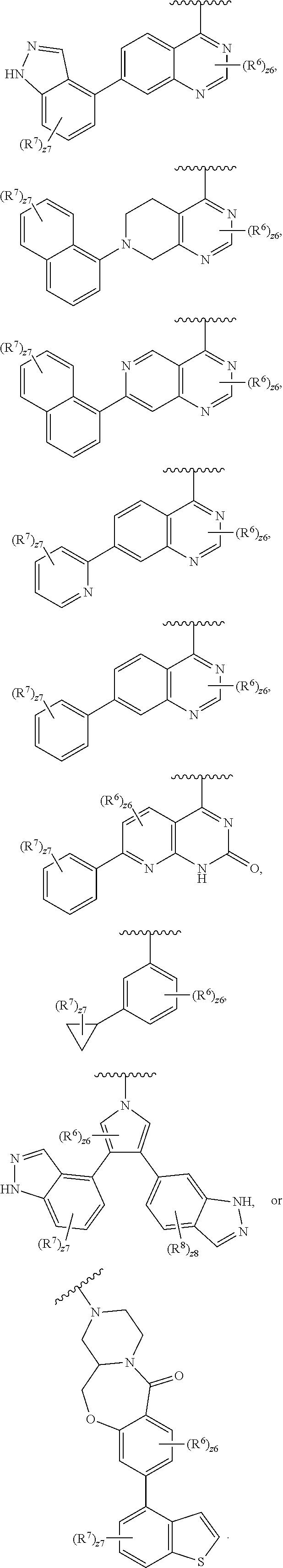

In embodiments, R¹ is

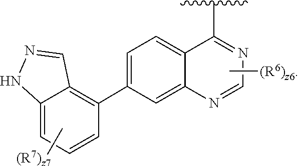

In embodiments, R¹ is

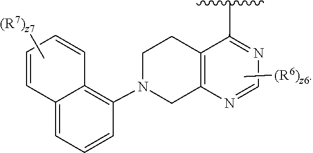

In embodiments, R¹ is

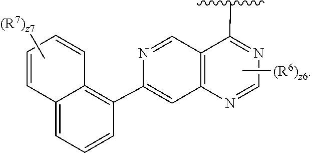

In embodiments, R¹ is

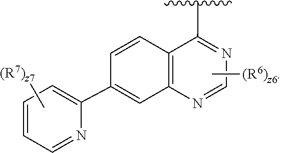

In embodiments, R¹ is

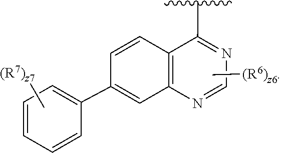

In embodiments, R¹ is

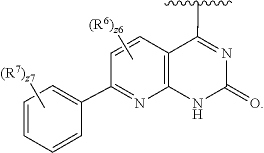

In embodiments, R¹ is

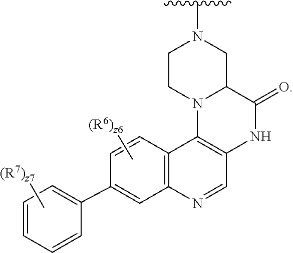

In embodiments, R¹ is

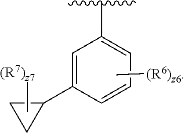

In embodiments, R¹ is

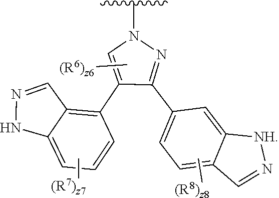

In embodiments, R¹ is

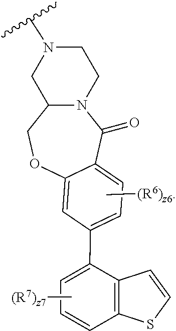

In embodiments, R¹ is

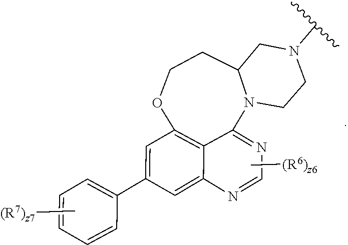

In embodiments, R¹ is

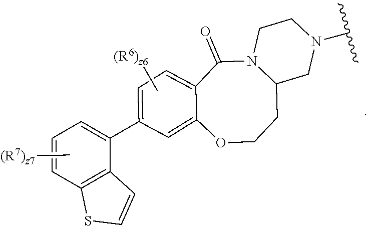

In embodiments, R¹ is

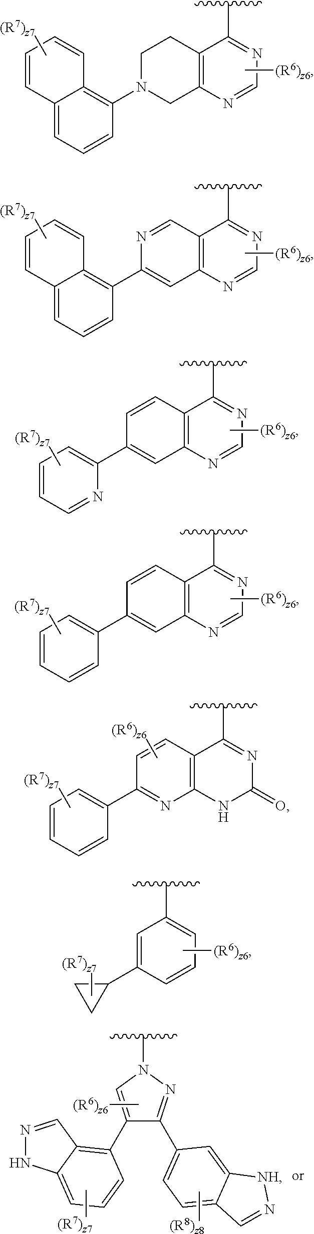

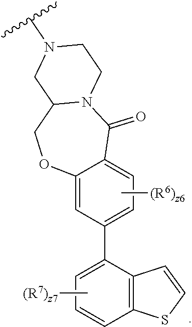

In embodiments, R¹ is

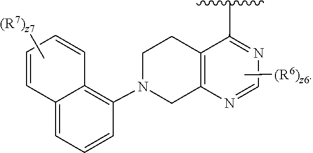

In embodiments, R¹ is

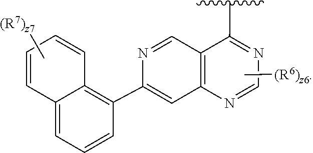

In embodiments, R¹ is

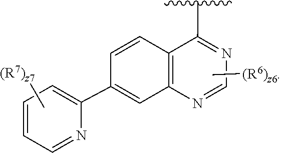

In embodiments, R¹ is

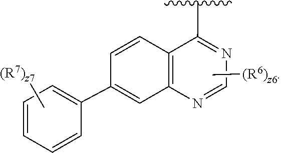

In embodiments, R¹ is

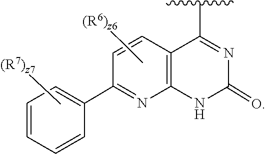

In embodiments, R¹ is

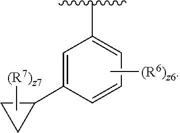

In embodiments, R¹ is

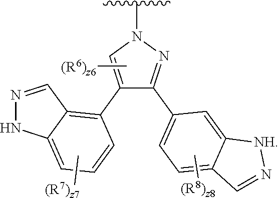

In embodiments, R¹ is

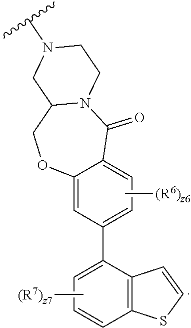

R⁶ is independently oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R⁷ is independently oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R⁵ is independently halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

The symbol z6 is an integer from 0 to 7.

The symbol z7 is an integer from 0 to 7.

The symbol z8 is an integer from 0 to 5.

In embodiments, a substituted R⁶ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁶ is substituted with a plurality of groups selected from substituent groups, size-limited substituent groups, and lower substituent groups; each substituent group, size-limited substituent group, and/or lower substituent group may optionally be different. In embodiments, when R⁶ is substituted, it is substituted with at least one substituent group. In embodiments, when R⁶ is substituted, it is substituted with at least one size-limited substituent group. In embodiments, when R⁶ is substituted, it is substituted with at least one lower substituent group.

In embodiments, R⁶ is independently halogen, —CCl₃, —CBr₃, —CF₃, —Cl₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, OCI₃, —OCH₂Cl, —OCH₂Br, —OCH₂F, —OCH₂I, —OCHCl₂, —OCHBr₂, —OCHF₂, —OCHI₂, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, R⁶ is independently oxo. In embodiments, R⁶ is independently halogen. In embodiments, R⁶ is independently —F. In embodiments, R⁶ is independently —Cl. In embodiments, R⁶ is independently —Br. In embodiments, R⁶ is independently —I. In embodiments, R⁶ is independently —CCl₃. In embodiments, R⁶ is independently —CBr₃. In embodiments, R⁶ is independently —CF₃. In embodiments, R⁶ is independently —CI₃. In embodiments, R⁶ is independently —CH₂Cl. In embodiments, R⁶ is independently —CH₂Br. In embodiments, R⁶ is independently —CH₂F. In embodiments, R⁶ is independently —CH₂I. In embodiments, R⁶ is independently —CHCl₂. In embodiments, R⁶ is independently —CHBr₂. In embodiments, R⁶ is independently —CHF₂. In embodiments, R⁶ is independently —CHI₂. In embodiments, R⁶ is independently —CN. In embodiments, R⁶ is independently —OH. In embodiments, R⁶ is independently —NH₂. In embodiments, R⁶ is independently —COOH. In embodiments, R⁶ is independently —CONH₂. In embodiments, R⁶ is independently —NO₂. In embodiments, R⁶ is independently —SH. In embodiments, R⁶ is independently —SO₃H. In embodiments, R⁶ is independently —OSO₃H. In embodiments, R⁶ is independently —SO₂NH₂. In embodiments, R⁶ is independently —NHNH₂. In embodiments, R⁶ is independently —ONH₂. In embodiments, R⁶ is independently —NHC(O)NHNH₂. In embodiments, R⁶ is independently —NHC(O)NH₂. In embodiments, R⁶ is independently —NHSO₂H. In embodiments, R⁶ is independently —NHC(O)H. In embodiments, R⁶ is independently —NHC(O)OH. In embodiments, R⁶ is independently —NHOH. In embodiments, R⁶ is independently —OCCl₃. In embodiments, R⁶ is independently —OCBr₃. In embodiments, R⁶ is independently —OCF₃. In embodiments, R⁶ is independently —OCI₃. In embodiments, R⁶ is independently —OCH₂Cl. In embodiments, R⁶ is independently —OCH₂Br. In embodiments, R⁶ is independently —OCH₂F. In embodiments, R⁶ is independently —OCH₂I. In embodiments, R⁶ is independently —OCHCl₂. In embodiments, R⁶ is independently —OCHBr₂. In embodiments, R⁶ is independently —OCHF₂. In embodiments, R⁶ is independently —OCHI₂. In embodiments, R⁶ is independently unsubstituted C₁-C₄ alkyl. In embodiments, R⁶ is independently unsubstituted methyl. In embodiments, R⁶ is independently unsubstituted ethyl. In embodiments, R⁶ is independently unsubstituted propyl. In embodiments, R⁶ is independently unsubstituted n-propyl. In embodiments, R⁶ is independently unsubstituted isopropyl. In embodiments, R⁶ is independently unsubstituted butyl. In embodiments, R⁶ is independently unsubstituted n-butyl. In embodiments, R⁶ is independently unsubstituted isobutyl. In embodiments, R⁶ is independently unsubstituted tert-butyl. In embodiments, R⁶ is independently substituted or unsubstituted 2 to 6 membered heteroalkyl. In embodiments, R⁶ is independently substituted 2 to 6 membered heteroalkyl. In embodiments, R⁶ is independently unsubstituted methoxy. In embodiments, R⁶ is independently unsubstituted ethoxy. In embodiments, R⁶ is independently unsubstituted propoxy. In embodiments, R⁶ is independently unsubstituted n-propoxy. In embodiments, R⁶ is independently unsubstituted isopropoxy. In embodiments, R⁶ is independently unsubstituted butoxy. In embodiments, R⁶ is independently unsubstituted n-butoxy. In embodiments, R⁶ is independently unsubstituted isobutoxy. In embodiments, R⁶ is independently unsubstituted tert-butoxy. In embodiments, R⁶ is independently substituted or unsubstituted phenyl. In embodiments, R⁶ is independently substituted or unsubstituted 5 to 6 membered heteroaryl. In embodiments, R⁶ is independently —O-alkyl-(substituted or unsubstituted heteterocycloalkyl). In embodiments, R⁶ is independently —O—CH₂-(substituted or unsubstituted heteterocycloalkyl). In embodiments, R⁶ is independently

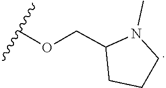 [SMILES: *OCC1CCCN1C]

In embodiments, R⁶ is independently

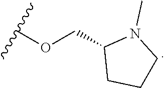 [SMILES: *OC[C@H]1CCCN1C]

In embodiments, R⁶ is independently

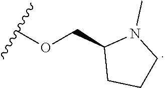 [SMILES: *OC[C@@H]1CCCN1C]

In embodiments, R⁶ is independently

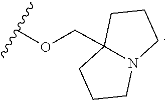 [SMILES: *OCC12CCCN1CCC2]

In embodiments, R⁶ is independently

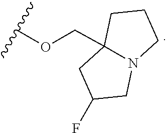 [SMILES: *OCC12CCCN1CC(F)C2]

In embodiments, R⁶ is independently

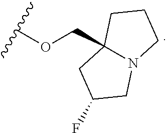 [SMILES: *OC[C@@]12CCCN1C[C@H](F)C2]

In embodiments, R⁶ is independently

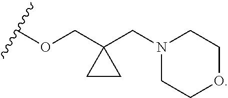 [SMILES: *OCC1(CN2CCOCC2)CC1]

In embodiments, R⁶ is independently

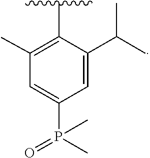 [SMILES: *c1c(C)cc(P(C)(C)=O)cc1C(C)C]

In embodiments, R⁶ is independently

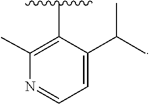 [SMILES: *c1c(C(C)C)ccnc1C]

In embodiments, R⁶ is independently

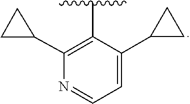 [SMILES: *c1c(C2CC2)ccnc1C1CC1]

In embodiments, R⁶ is independently a halogen, —OH, unsubstituted C₁-C₄ alkyl, substituted 2 to 6 membered heteroalkyl, or substituted 5 to 6 membered heteroaryl. In embodiments, R⁶ is independently —F, —Cl, —OH, or unsubstituted methyl. In embodiments, R⁶ is independently a 2 to 6 membered heteroalkyl, substituted with substituted heterocycloalkyl or unsubstituted fused heterocycloalkyl. In embodiments, R⁶ is independently a substituted pyridyl.

In embodiments, z6 is 0. In embodiments, z6 is 1. In embodiments, z6 is 2. In embodiments, z6 is 3. In embodiments, z6 is 4. In embodiments, z6 is 5. In embodiments, z6 is 6. In embodiments, z6 is 7.

In embodiments, a substituted R⁷ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁷ is substituted with a plurality of groups selected from substituent groups, size-limited substituent groups, and lower substituent groups; each substituent group, size-limited substituent group, and/or lower substituent group may optionally be different. In embodiments, when R⁷ is substituted, it is substituted with at least one substituent group. In embodiments, when R⁷ is substituted, it is substituted with at least one size-limited substituent group. In embodiments, when R⁷ is substituted, it is substituted with at least one lower substituent group.

In embodiments, R⁷ is independently halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, R⁷ is independently oxo. In embodiments, R⁷ is independently halogen. In embodiments, R⁷ is independently —F. In embodiments, R⁷ is independently —Cl. In embodiments, R⁷ is independently —Br. In embodiments, R⁷ is independently —I. In embodiments, R⁷ is independently —CCl₃. In embodiments, R⁷ is independently —CBr₃. In embodiments, R⁷ is independently —CF₃. In embodiments, R⁷ is independently —Cl₃. In embodiments, R⁷ is independently —CH₂Cl. In embodiments, R⁷ is independently —CH₂Br. In embodiments, R⁷ is independently —CH₂F. In embodiments, R⁷ is independently —CH₂I. In embodiments, R⁷ is independently —CHCl₂. In embodiments, R⁷ is independently —CHBr₂. In embodiments, R⁷ is independently —CHF₂. In embodiments, R⁷ is independently —CHI₂. In embodiments, R⁷ is independently —CN. In embodiments, R⁷ is independently —OH. In embodiments, R⁷ is independently —NH₂. In embodiments, R⁷ is independently —COOH. In embodiments, R⁷ is independently —CONH₂. In embodiments, R⁷ is independently —NO₂. In embodiments, R⁷ is independently —SH. In embodiments, R⁷ is independently —SO₃H. In embodiments, R⁷ is independently —OSO₃H. In embodiments, R⁷ is independently —SO₂NH₂. In embodiments, R⁷ is independently —NHNH₂. In embodiments, R⁷ is independently —ONH₂. In embodiments, R⁷ is independently —NHC(O)NHNH₂. In embodiments, R⁷ is independently —NHC(O)NH₂. In embodiments, R⁷ is independently —NHSO₂H. In embodiments, R⁷ is independently —NHC(O)H. In embodiments, R⁷ is independently —NHC(O)OH. In embodiments, R⁷ is independently —NHOH. In embodiments, R⁷ is independently —OCCl₃. In embodiments, R⁷ is independently —OCBr₃. In embodiments, R⁷ is independently —OCF₃. In embodiments, R⁷ is independently —OCI₃. In embodiments, R⁷ is independently —OCH₂Cl. In embodiments, R⁷ is independently —OCH₂Br. In embodiments, R⁷ is independently —OCH₂F. In embodiments, R⁷ is independently —OCH₂I. In embodiments, R⁷ is independently —OCHCl₂. In embodiments, R⁷ is independently —OCHBr₂. In embodiments, R⁷ is independently —OCHF₂. In embodiments, R⁷ is independently —OCHI₂. In embodiments, R⁷ is independently unsubstituted C₁-C₄ alkyl. In embodiments, R⁷ is independently unsubstituted methyl. In embodiments, R⁷ is independently unsubstituted ethyl. In embodiments, R⁷ is independently unsubstituted propyl. In embodiments, R⁷ is independently unsubstituted n-propyl. In embodiments, R⁷ is independently unsubstituted isopropyl. In embodiments, R⁷ is independently unsubstituted butyl. In embodiments, R⁷ is independently unsubstituted n-butyl. In embodiments, R⁷ is independently unsubstituted isobutyl. In embodiments, R⁷ is independently unsubstituted tert-butyl. In embodiments, R⁷ is independently unsubstituted C₂-C₄ alkynyl. In embodiments, R⁷ is independently unsubstituted ethynyl. In embodiments, R⁷ is independently unsubstituted propynyl. In embodiments, R⁷ is independently unsubstituted butynyl. In embodiments, R⁷ is independently substituted or unsubstituted 2 to 6 membered heteroalkyl. In embodiments, R⁷ is independently unsubstituted methoxy. In embodiments, R⁷ is independently unsubstituted ethoxy. In embodiments, R⁷ is independently unsubstituted propoxy. In embodiments, R⁷ is independently unsubstituted n-propoxy. In embodiments, R⁷ is independently unsubstituted isopropoxy. In embodiments, R⁷ is independently unsubstituted butoxy. In embodiments, R⁷ is independently unsubstituted n-butoxy. In embodiments, R⁷ is independently unsubstituted isobutoxy. In embodiments, R⁷ is independently unsubstituted tert-butoxy.

In embodiments, R⁷ is independently a halogen, —CF₃, —CN, —OH, —NH₂, or unsubstituted C₁-C₄ alkyl. In embodiments, R⁷ is independently —F, —Cl, —CF₃, —CN, —OH, —NH₂, or unsubstituted methyl.

In embodiments, R⁷ is independently a halogen, —CF₃, —CN, —OH, —NH₂, unsubstituted C₁-C₄ alkyl, or unsubstituted C₂-C₄ alkynyl. In embodiments, R⁷ is independently —F, —Cl, —CF₃, —CN, —OH, —NH₂, unsubstituted methyl, or unsubstituted ethynyl.

In embodiments, z7 is 0. In embodiments, z7 is 1. In embodiments, z7 is 2. In embodiments, z7 is 3. In embodiments, z7 is 4. In embodiments, z7 is 5. In embodiments, z7 is 6. In embodiments, z7 is 7.

In embodiments, a substituted R⁸ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁸ is substituted with a plurality of groups selected from substituent groups, size-limited substituent groups, and lower substituent groups; each substituent group, size-limited substituent group, and/or lower substituent group may optionally be different. In embodiments, when R⁸ is substituted, it is substituted with at least one substituent group. In embodiments, when R⁸ is substituted, it is substituted with at least one size-limited substituent group. In embodiments, when R⁸ is substituted, it is substituted with at least one lower substituent group.

In embodiments, R⁸ is independently halogen. In embodiments, R⁸ is independently —F. In embodiments, R⁸ is independently —Cl. In embodiments, R⁸ is independently —Br. In embodiments, R⁸ is independently —I. In embodiments, R⁸ is independently —CCl₃. In embodiments, R⁸ is independently —CBr₃. In embodiments, R⁸ is independently —CF₃. In embodiments, R⁸ is independently —CI₃. In embodiments, R⁸ is independently —CH₂Cl. In embodiments, R⁸ is independently —CH₂Br. In embodiments, R⁸ is independently —CH₂F. In embodiments, R⁸ is independently —CH₂I. In embodiments, R⁸ is independently —CHCl₂. In embodiments, R⁸ is independently —CHBr₂. In embodiments, R⁸ is independently —CHF₂. In embodiments, R⁸ is independently —CHI₂. In embodiments, R⁸ is independently —CN. In embodiments, R⁸ is independently —OH. In embodiments, R⁸ is independently —NH₂. In embodiments, R⁸ is independently —COOH. In embodiments, R⁸ is independently —CONH₂. In embodiments, R⁸ is independently —NO₂. In embodiments, R⁸ is independently —SH. In embodiments, R⁸ is independently —SO₃H. In embodiments, R⁸ is independently —OSO₃H. In embodiments, R⁸ is independently —SO₂NH₂. In embodiments, R⁸ is independently —NHNH₂. In embodiments, R⁸ is independently —ONH₂. In embodiments, R⁸ is independently —NHC(O)NHNH₂. In embodiments, R⁸ is independently —NHC(O)NH₂. In embodiments, R⁸ is independently —NHSO₂H. In embodiments, R⁸ is independently —NHC(O)H. In embodiments, R⁸ is independently —NHC(O)OH. In embodiments, R⁸ is independently —NHOH. In embodiments, R⁸ is independently —OCCl₃. In embodiments, R⁸ is independently —OCBr₃. In embodiments, R⁸ is independently —OCF₃. In embodiments, R⁸ is independently —OCI₃. In embodiments, R⁸ is independently —OCH₂Cl. In embodiments, R⁸ is independently —OCH₂Br. In embodiments, R⁸ is independently —OCH₂F. In embodiments, R⁸ is independently —OCH₂I. In embodiments, R⁸ is independently —OCHCl₂. In embodiments, R⁸ is independently —OCHBr₂. In embodiments, R⁸ is independently —OCHF₂. In embodiments, R⁸ is independently —OCHI₂. In embodiments, R⁸ is independently unsubstituted C₁-C₄ alkyl. In embodiments, R⁸ is independently unsubstituted methyl. In embodiments, R⁸ is independently unsubstituted ethyl. In embodiments, R⁸ is independently unsubstituted propyl. In embodiments, R⁸ is independently unsubstituted n-propyl. In embodiments, R⁸ is independently unsubstituted isopropyl. In embodiments, R⁸ is independently unsubstituted butyl. In embodiments, R⁸ is independently unsubstituted n-butyl. In embodiments, R⁸ is independently unsubstituted isobutyl. In embodiments, R⁸ is independently unsubstituted tert-butyl. In embodiments, R⁸ is independently substituted or unsubstituted 2 to 6 membered heteroalkyl. In embodiments, R⁸ is independently unsubstituted methoxy. In embodiments, R⁸ is independently unsubstituted ethoxy. In embodiments, R⁸ is independently unsubstituted propoxy. In embodiments, R⁸ is independently unsubstituted n-propoxy. In embodiments, R⁸ is independently unsubstituted isopropoxy. In embodiments, R⁸ is independently unsubstituted butoxy. In embodiments, R⁸ is independently unsubstituted n-butoxy. In embodiments, R⁸ is independently unsubstituted isobutoxy. In embodiments, R⁸ is independently unsubstituted tert-butoxy.

In embodiments, R⁸ is independently a halogen or unsubstituted C₁-C₄ alkyl. In embodiments, R⁸ is independently —Cl or unsubstituted methyl.

In embodiments, z8 is 0. In embodiments, z8 is 1. In embodiments, z8 is 2. In embodiments, z8 is 3. In embodiments, z8 is 4. In embodiments, z8 is 5.

In embodiments, R¹ is

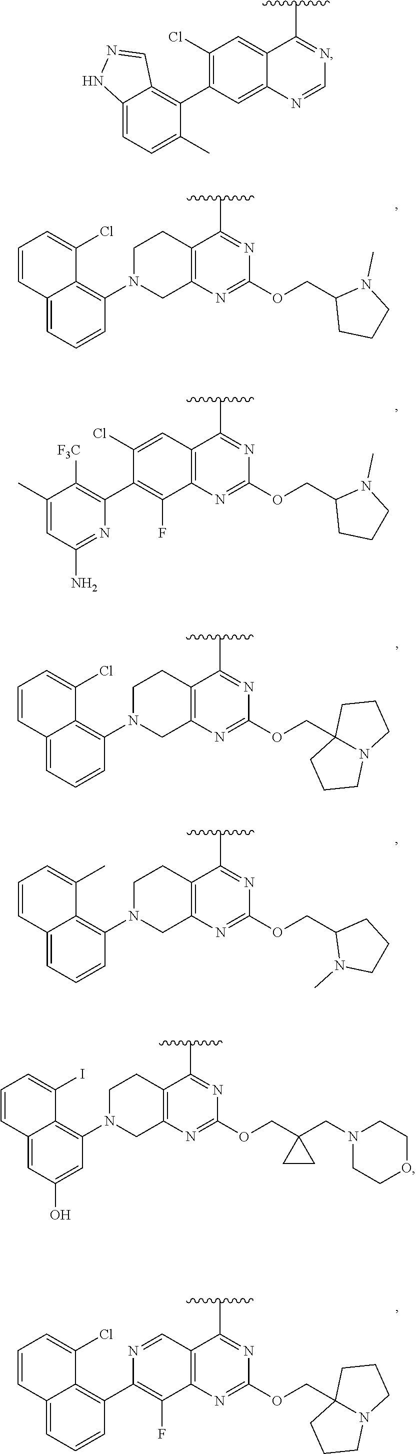

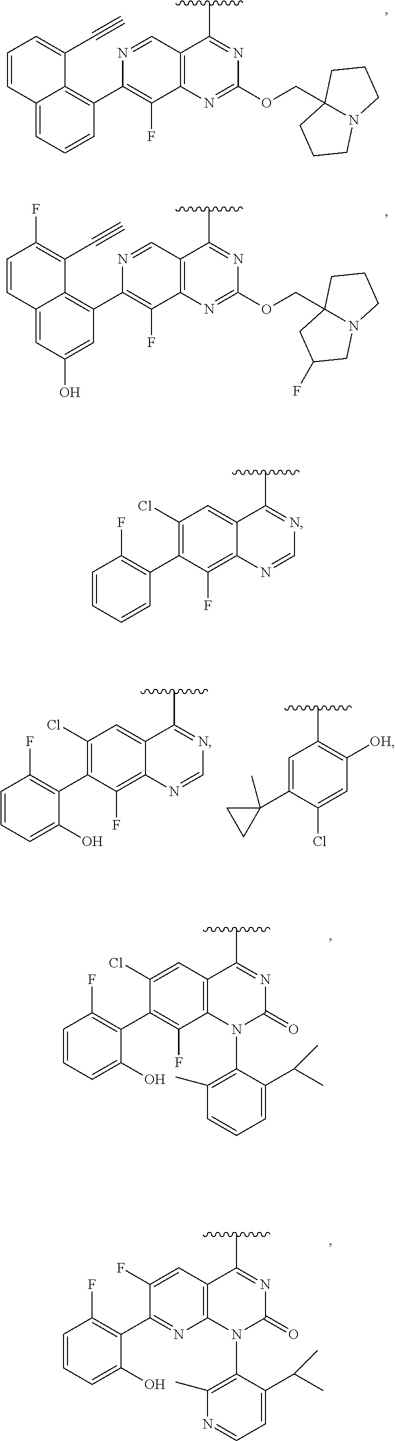

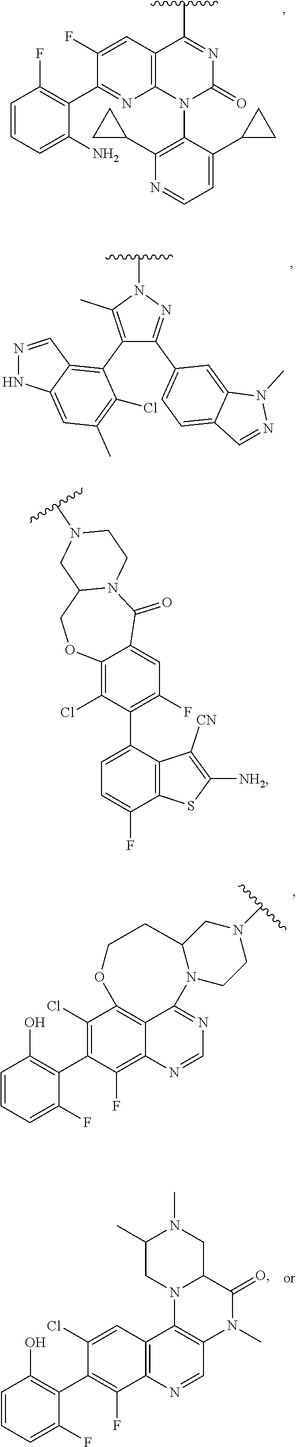 [SMILES: *c1nc(=O)n(-c2c(C3CC3)ccnc2C2CC2)c2nc(-c3c(N)cccc3F)c(F)cc12 ; *n1nc(-c2ccc3cnn(C)c3c2)c(-c2c(Cl)c(C)cc3[nH]ncc23)c1C ; *N1CCN2C(=O)c3cc(F)c(-c4ccc(F)c5sc(N)c(C#N)c45)c(Cl)c3OCC2C1 ; *N1CCN2c3ncnc4c(F)c(-c5c(O)cccc5F)c(Cl)c(c34)OCCC2C1 ; CC1CN2c3c(cnc4c(F)c(-c5c(O)cccc5F)c(Cl)cc34)N(C)C(=O)C2CN1C]

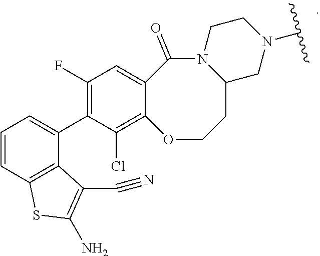 [SMILES: *N1CCN2C(=O)c3cc(F)c(-c4cccc5sc(N)c(C#N)c45)c(Cl)c3OCCC2C1]

In embodiments, R¹ is

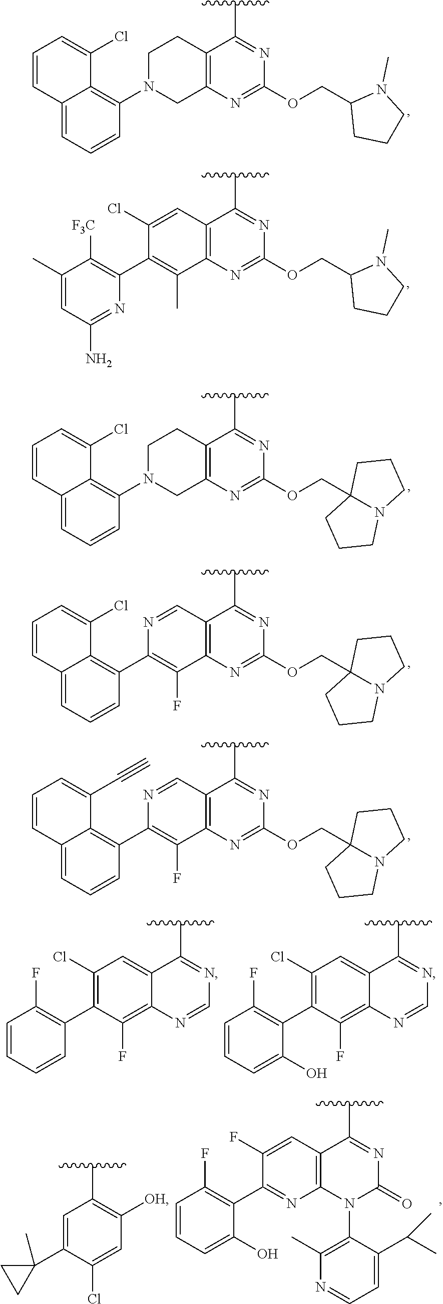

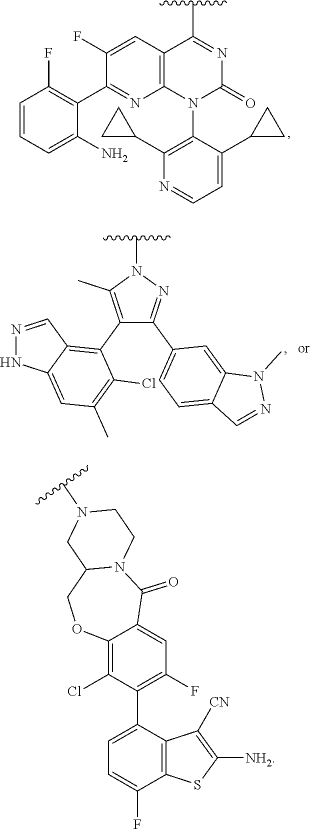 [SMILES: *c1nc(=O)n(-c2c(C3CC3)ccnc2C2CC2)c2nc(-c3c(N)cccc3F)c(F)cc12 ; *n1nc(-c2ccc3cnn(C)c3c2)c(-c2c(Cl)c(C)cc3[nH]ncc23)c1C ; *N1CCN2C(=O)c3cc(F)c(-c4ccc(F)c5sc(N)c(C#N)c45)c(Cl)c3OCC2C1]

In embodiments, R¹ is

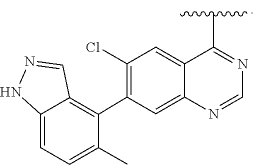 [SMILES: *c1ncnc2cc(-c3c(C)ccc4[nH]ncc34)c(Cl)cc12]

In embodiments, R¹ is

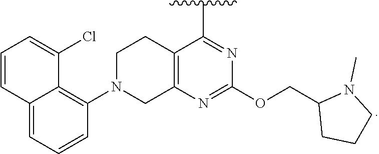 [SMILES: *c1nc(OCC2CCCN2C)nc2c1CCN(c1cccc3cccc(Cl)c13)C2]

In embodiments, R¹ is

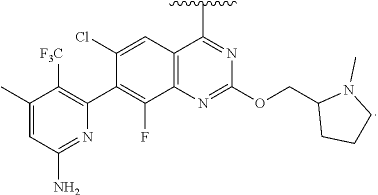 [SMILES: *c1nc(OCC2CCCN2C)nc2c(F)c(-c3nc(N)cc(C)c3C(F)(F)F)c(Cl)cc12]

In embodiments, R¹ is

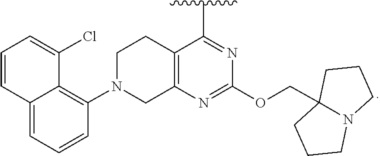 [SMILES: *c1nc(OCC23CCCN2CCC3)nc2c1CCN(c1cccc3cccc(Cl)c13)C2]

In embodiments, R¹ is

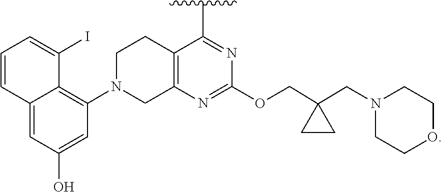 [SMILES: *c1nc(OCC2(CN3CCOCC3)CC2)nc2c1CCN(c1cc(O)cc3cccc(I)c13)C2]

In embodiments, R¹ is

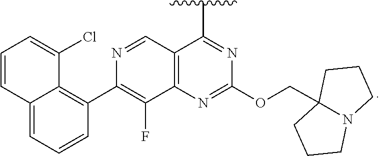 [SMILES: *c1nc(OCC23CCCN2CCC3)nc2c(F)c(-c3cccc4cccc(Cl)c34)ncc12]

In embodiments, R¹ is

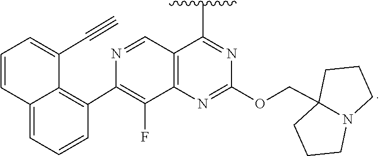 [SMILES: *c1nc(OCC23CCCN2CCC3)nc2c(F)c(-c3cccc4cccc(C#C)c34)ncc12]

In embodiments, R¹ is

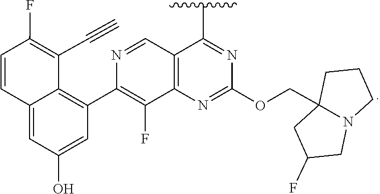 [SMILES: *c1nc(OCC23CCCN2CC(F)C3)nc2c(F)c(-c3cc(O)cc4ccc(F)c(C#C)c34)ncc12]

In embodiments, R¹ is

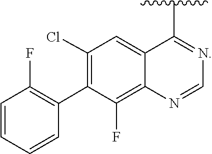 [SMILES: *c1ncnc2c(F)c(-c3ccccc3F)c(Cl)cc12]

In embodiments, R¹ is

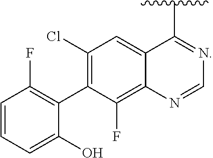 [SMILES: *c1ncnc2c(F)c(-c3c(O)cccc3F)c(Cl)cc12]

In embodiments, R¹ is

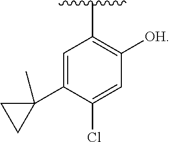 [SMILES: *c1cc(C2(C)CC2)c(Cl)cc1O]

In embodiments, R¹ is

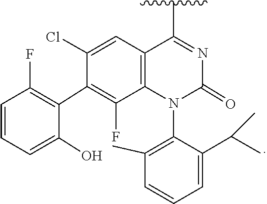 [SMILES: *c1nc(=O)n(-c2c(C)cccc2C(C)C)c2c(F)c(-c3c(O)cccc3F)c(Cl)cc12]

In embodiments, R¹ is

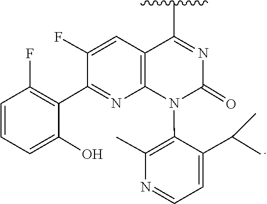 [SMILES: *c1nc(=O)n(-c2c(C(C)C)ccnc2C)c2nc(-c3c(O)cccc3F)c(F)cc12]

In embodiments, R¹ is

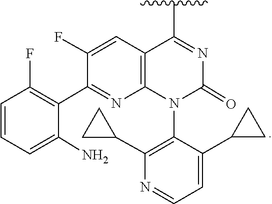 [SMILES: *c1nc(=O)n(-c2c(C3CC3)ccnc2C2CC2)c2nc(-c3c(N)cccc3F)c(F)cc12]

In embodiments, R¹ is

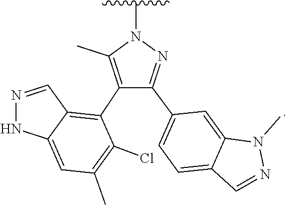 [SMILES: *n1nc(-c2ccc3cnn(C)c3c2)c(-c2c(Cl)c(C)cc3[nH]ncc23)c1C]

In embodiments, R¹ is

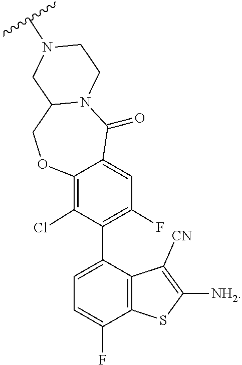 [SMILES: *N1CCN2C(=O)c3cc(F)c(-c4ccc(F)c5sc(N)c(C#N)c45)c(Cl)c3OCC2C1]

In embodiments, R¹ is

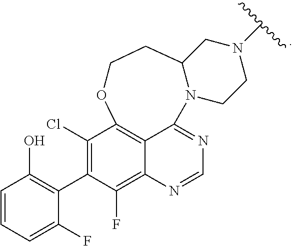 [SMILES: *N1CCN2c3ncnc4c(F)c(-c5c(O)cccc5F)c(Cl)c(c34)OCCC2C1]

In embodiments, R¹ is

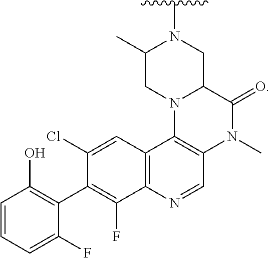 [SMILES: *N1CC2C(=O)N(C)c3cnc4c(F)c(-c5c(O)cccc5F)c(Cl)cc4c3N2CC1C]

In embodiments, R¹ is

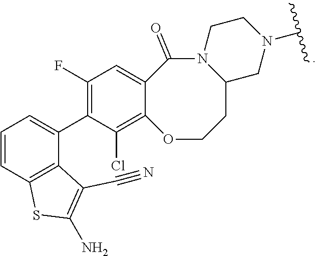 [SMILES: *N1CCN2C(=O)c3cc(F)c(-c4cccc5sc(N)c(C#N)c45)c(Cl)c3OCCC2C1]

In embodiments, R¹ is

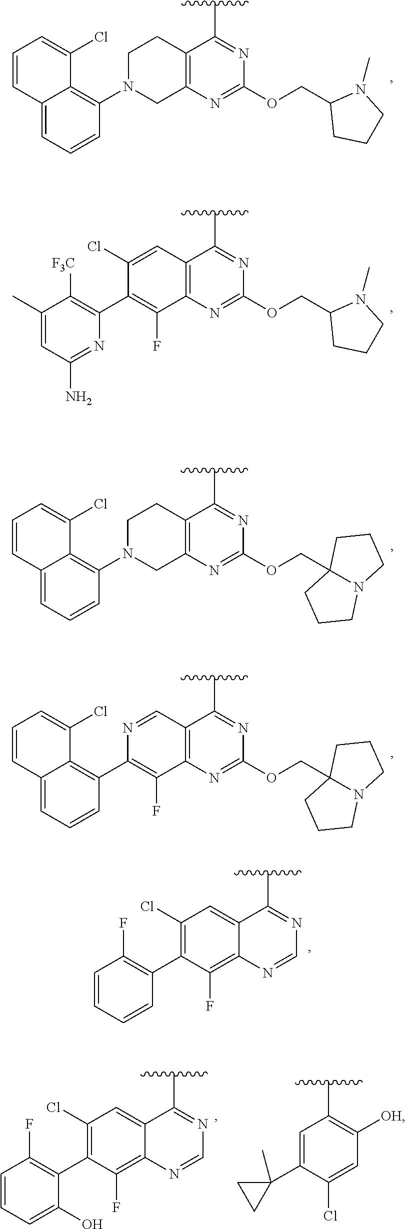

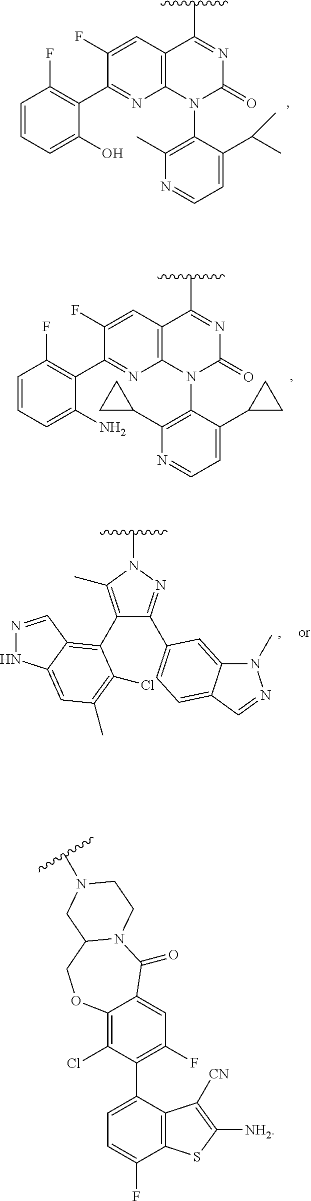 [SMILES: *c1nc(=O)n(-c2c(C(C)C)ccnc2C)c2nc(-c3c(O)cccc3F)c(F)cc12 ; *c1nc(=O)n(-c2c(C3CC3)ccnc2C2CC2)c2nc(-c3c(N)cccc3F)c(F)cc12 ; *n1nc(-c2ccc3cnn(C)c3c2)c(-c2c(Cl)c(C)cc3[nH]ncc23)c1C ; *N1CCN2C(=O)c3cc(F)c(-c4ccc(F)c5sc(N)c(C#N)c45)c(Cl)c3OCC2C1]

In embodiments, R¹ is

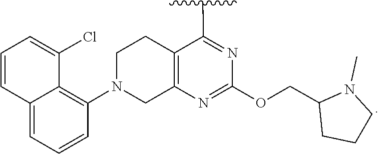 [SMILES: *c1nc(OCC2CCCN2C)nc2c1CCN(c1cccc3cccc(Cl)c13)C2]

In embodiments, R¹ is

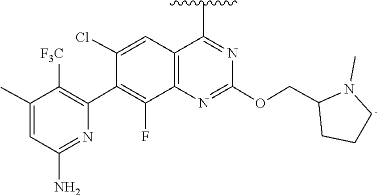 [SMILES: *c1nc(OCC2CCCN2C)nc2c(F)c(-c3nc(N)cc(C)c3C(F)(F)F)c(Cl)cc12]

In embodiments, R¹ is

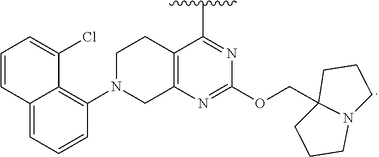 [SMILES: *c1nc(OCC23CCCN2CCC3)nc2c1CCN(c1cccc3cccc(Cl)c13)C2]

In embodiments, R¹ is

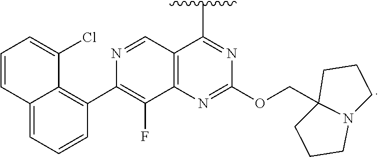 [SMILES: *c1nc(OCC23CCCN2CCC3)nc2c(F)c(-c3cccc4cccc(Cl)c34)ncc12]

In embodiments, R¹ is

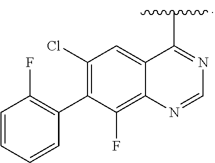 [SMILES: *c1ncnc2c(F)c(-c3ccccc3F)c(Cl)cc12]

In embodiments, R¹ is

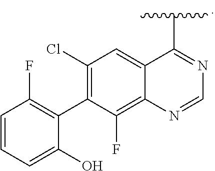 [SMILES: *c1ncnc2c(F)c(-c3c(O)cccc3F)c(Cl)cc12]

In embodiments, R¹ is

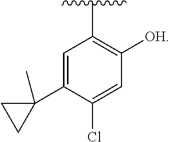 [SMILES: *c1cc(C2(C)CC2)c(Cl)cc1O]

In embodiments, R¹ is

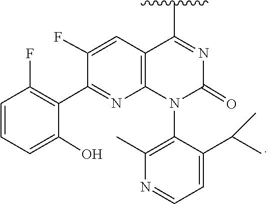 [SMILES: *c1nc(=O)n(-c2c(C(C)C)ccnc2C)c2nc(-c3c(O)cccc3F)c(F)cc12]

In embodiments, R¹ is

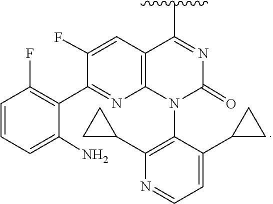 [SMILES: *c1nc(=O)n(-c2c(C3CC3)ccnc2C2CC2)c2nc(-c3c(N)cccc3F)c(F)cc12]

In embodiments, R¹ is

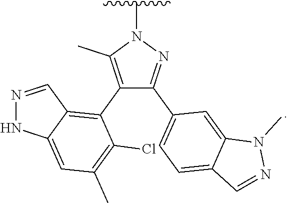 [SMILES: *n1nc(-c2ccc3cnn(C)c3c2)c(-c2c(Cl)c(C)cc3[nH]ncc23)c1C]

In embodiments, R¹ is

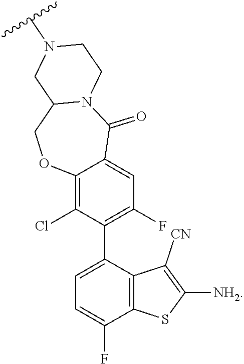 [SMILES: *N1CCN2C(=O)c3cc(F)c(-c4ccc(F)c5sc(N)c(C#N)c45)c(Cl)c3OCC2C1]

In embodiments, R¹ is a monovalent form of ARS-1620. In embodiments, R¹ is a monovalent form of

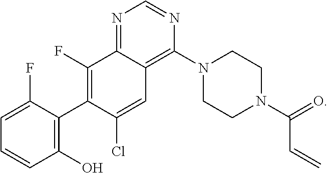 [SMILES: C=CC(=O)N1CCN(c2ncnc3c(F)c(-c4c(O)cccc4F)c(Cl)cc23)CC1]

In embodiments, R¹ is

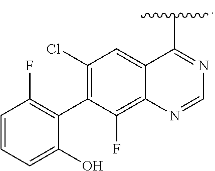 [SMILES: *c1ncnc2c(F)c(-c3c(O)cccc3F)c(Cl)cc12]

In embodiments, R¹ is a monovalent form of a portion of ARS-1620, wherein R¹ does not include the substituted piperazinyl moiety.

In embodiments, R¹ is a monovalent form of AMG-510. In embodiments, R¹ is a monovalent form of a compound as described in Canon, J. et al. Nature 575, 217-223 (2019), which is herein incorporated by reference in its entirety for all purposes. In embodiments, R¹ is a monovalent form of

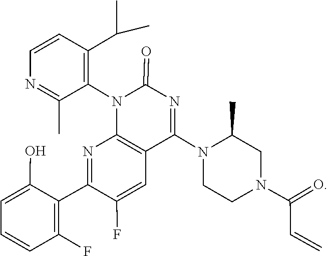 [SMILES: C=CC(=O)N1CCN(c2nc(=O)n(-c3c(C(C)C)ccnc3C)c3nc(-c4c(O)cccc4F)c(F)cc23)[C@@H](C)C1]

In embodiments, R¹ is

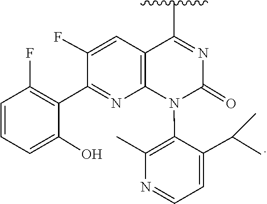 [SMILES: *c1nc(=O)n(-c2c(C(C)C)ccnc2C)c2nc(-c3c(O)cccc3F)c(F)cc12]

In embodiments, R¹ is a monovalent form of a portion of AMG-510, wherein R¹ does not include the substituted piperazinyl moiety or equivalent for compounds described in Canon, et al.

In embodiments, R¹ is a monovalent form of MRTX-849. In embodiments, R¹ is a monovalent form of a compound as described in Fell, J. B. et al. J. Med. Chem. 63, 6679-6693 (2020), which is herein incorporated by reference in its entirety for all purposes. In embodiments, R¹ is a monovalent form of

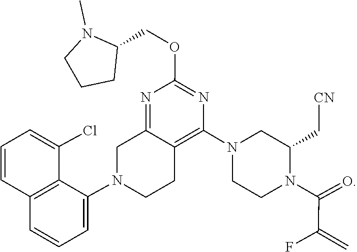 [SMILES: C=C(F)C(=O)N1CCN(c2nc(OC[C@@H]3CCCN3C)nc3c2CCN(c2cccc4cccc(Cl)c24)C3)C[C@@H]1CC#N]

In embodiments, R¹ is

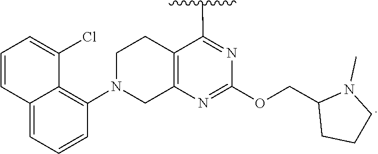 [SMILES: *c1nc(OCC2CCCN2C)nc2c1CCN(c1cccc3cccc(Cl)c13)C2]

In embodiments, R¹ is

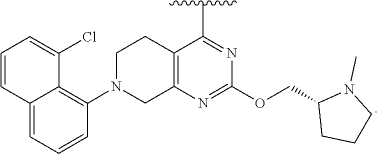 [SMILES: *c1nc(OC[C@H]2CCCN2C)nc2c1CCN(c1cccc3cccc(Cl)c13)C2]

In embodiments, R¹ is

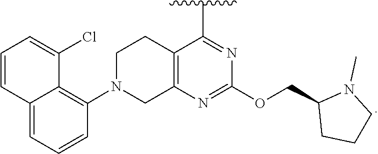 [SMILES: *c1nc(OC[C@@H]2CCCN2C)nc2c1CCN(c1cccc3cccc(Cl)c13)C2]

In embodiments, R¹ is a monovalent form of a portion of MRTX-849, wherein R¹ does not include the substituted piperazinyl moiety or equivalent for compounds described in Fell, et al.

In embodiments, R¹ is a monovalent form of GDC-6036. In embodiments, R¹ is a monovalent form of a compound as described in WO2020097537, which is herein incorporated by reference in its entirety for all purposes. In embodiments, R¹ is a monovalent form of

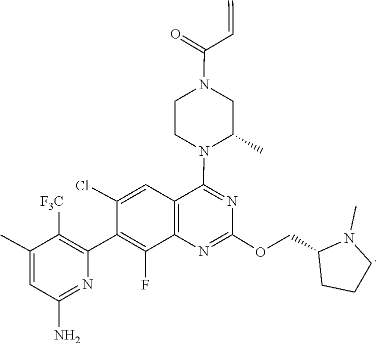 [SMILES: C=CC(=O)N1CCN(c2nc(OC[C@H]3CCCN3C)nc3c(F)c(-c4nc(N)cc(C)c4C(F)(F)F)c(Cl)cc23)[C@@H](C)C1]

In embodiments, R¹ is

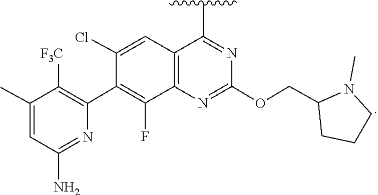 [SMILES: *c1nc(OCC2CCCN2C)nc2c(F)c(-c3nc(N)cc(C)c3C(F)(F)F)c(Cl)cc12]

In embodiments, R¹ is

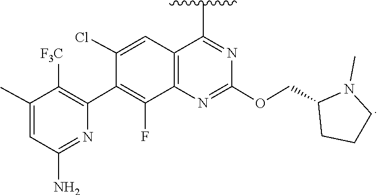 [SMILES: *c1nc(OC[C@H]2CCCN2C)nc2c(F)c(-c3nc(N)cc(C)c3C(F)(F)F)c(Cl)cc12]

In embodiments, R¹ is

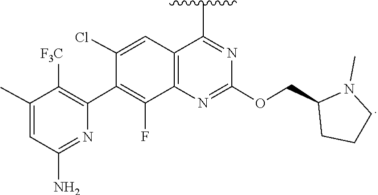 [SMILES: *c1nc(OC[C@@H]2CCCN2C)nc2c(F)c(-c3nc(N)cc(C)c3C(F)(F)F)c(Cl)cc12]

In embodiments, R¹ is a monovalent form of a portion of GDC-6036, wherein R¹ does not include the substituted piperazinyl moiety or equivalent for compounds described in WO2020097537.

In embodiments, R¹ is a monovalent form of MRTX1133. In embodiments, R¹ is a monovalent form of a compound as described in Wang, X. et al. J. Med. Chem. 65, 3123-3133 (2022), which is herein incorporated by reference in its entirety for all purposes. In embodiments, R¹ is a monovalent form of

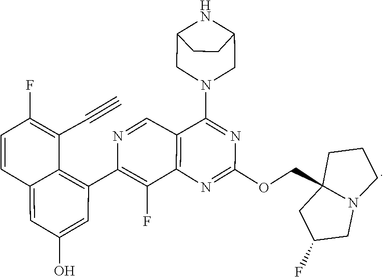 [SMILES: C#Cc1c(F)ccc2cc(O)cc(-c3ncc4c(N5CC6CCC(C5)N6)nc(OC[C@@]56CCCN5C[C@H](F)C6)nc4c3F)c12]

In embodiments, R¹ is

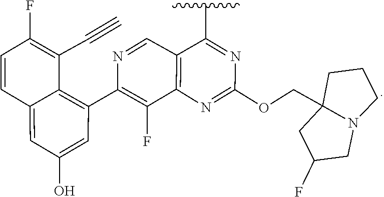 [SMILES: *c1nc(OCC23CCCN2CC(F)C3)nc2c(F)c(-c3cc(O)cc4ccc(F)c(C#C)c34)ncc12]

In embodiments, R¹ is

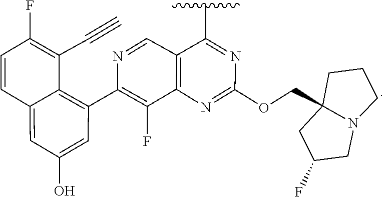 [SMILES: *c1nc(OC[C@@]23CCCN2C[C@H](F)C3)nc2c(F)c(-c3cc(O)cc4ccc(F)c(C#C)c34)ncc12]

In embodiments, R¹ is a monovalent form of a portion of MRTX1133, wherein R¹ does not include the diazabicyclooctanyl moiety or equivalent for compounds described in Wang, et al.

In embodiments, R¹ is a monovalent form of JDQ-443. In embodiments, R¹ is a monovalent form of a compound as described in WO2021120890, which is herein incorporated by reference in its entirety for all purposes. In embodiments, R¹ is a monovalent form of

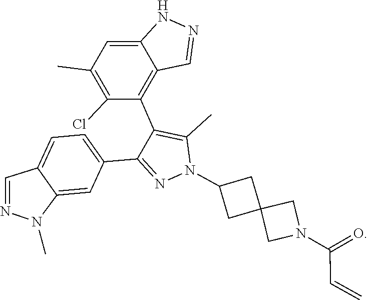 [SMILES: C=CC(=O)N1CC2(CC(n3nc(-c4ccc5cnn(C)c5c4)c(-c4c(Cl)c(C)cc5[nH]ncc45)c3C)C2)C1]

In embodiments, R¹ is

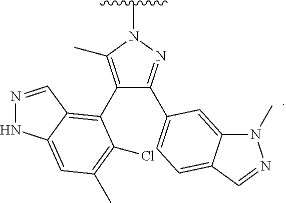 [SMILES: *n1nc(-c2ccc3cnn(C)c3c2)c(-c2c(Cl)c(C)cc3[nH]ncc23)c1C]

In embodiments, R¹ is a monovalent form of a portion of JDQ-443, wherein R¹ does not include the azaspiroheptanyl moiety or equivalent for compounds described in WO2021120890.

In embodiments, R¹ is a monovalent form of a compound as described in WO2021118877, which is herein incorporated by reference in its entirety for all purposes. In embodiments, R¹ is

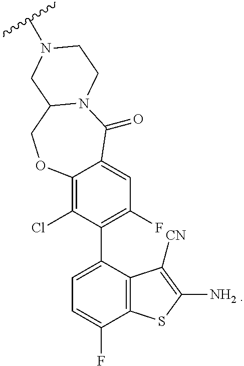 [SMILES: *N1CCN2C(=O)c3cc(F)c(-c4ccc(F)c5sc(N)c(C#N)c45)c(Cl)c3OCC2C1]

In embodiments, R¹ is a monovalent form of a portion of a compound described in WO2021118877, wherein R¹ does not include the acryloyl moiety or equivalent for compounds described in WO2021118877.

In embodiments, R¹ is a monovalent form of a compound as described in WO2021120045, which is herein incorporated by reference in its entirety for all purposes. In embodiments, R¹ is

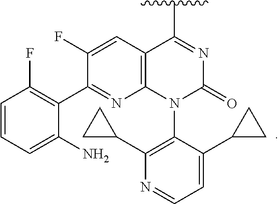 [SMILES: *c1nc(=O)n(-c2c(C3CC3)ccnc2C2CC2)c2nc(-c3c(N)cccc3F)c(F)cc12]

In embodiments, R¹ is a monovalent form of a portion of a compound described in WO2021120045, wherein R¹ does not include the substituted piperazinyl moiety or equivalent for compounds described in WO2021120045.

In embodiments, R¹ is a monovalent form of sotorasib. In embodiments, R¹ is a monovalent form of a compound as described in U.S. Pat. Nos. 10,519,146, 11,236,091, and 11,426,404, which are herein incorporated by reference in their entirety for all purposes. In embodiments, R¹ is a monovalent form of

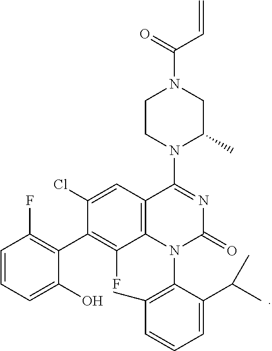 [SMILES: C=CC(=O)N1CCN(c2nc(=O)n(-c3c(C)cccc3C(C)C)c3c(F)c(-c4c(O)cccc4F)c(Cl)cc23)[C@@H](C)C1]

In embodiments, R¹ is

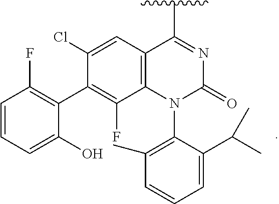 [SMILES: *c1nc(=O)n(-c2c(C)cccc2C(C)C)c2c(F)c(-c3c(O)cccc3F)c(Cl)cc12]

In embodiments, R¹ is a monovalent form of a portion of sotorasib, wherein R¹ does not include the substituted piperazinyl moiety or equivalent for compounds described in U.S. Pat. Nos. 10,519,146, 11,236,091, and 11,426,404.

In embodiments, R¹ is a monovalent form of adagrasib. In embodiments, R¹ is a monovalent form of a compound as described in WO 2021/037018, which is herein incorporated by reference in its entirety for all purposes. In embodiments, R¹ is a monovalent form of

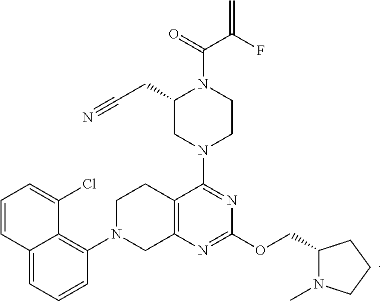 [SMILES: C=C(F)C(=O)N1CCN(c2nc(OC[C@@H]3CCCN3C)nc3c2CCN(c2cccc4cccc(Cl)c24)C3)C[C@@H]1CC#N]

In embodiments, R¹ is

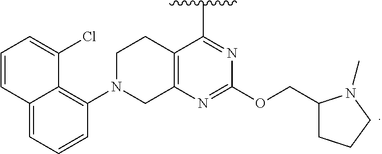 [SMILES: *c1nc(OCC2CCCN2C)nc2c1CCN(c1cccc3cccc(Cl)c13)C2]

In embodiments, R¹ is

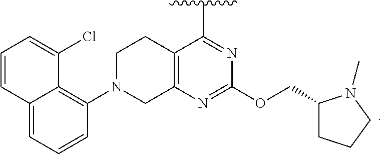 [SMILES: *c1nc(OC[C@H]2CCCN2C)nc2c1CCN(c1cccc3cccc(Cl)c13)C2]

In embodiments, R¹ is

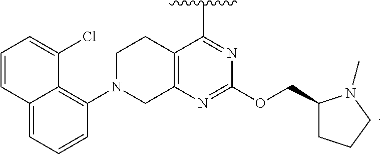 [SMILES: *c1nc(OC[C@@H]2CCCN2C)nc2c1CCN(c1cccc3cccc(Cl)c13)C2]

In embodiments, R¹ is a monovalent form of a portion of adagrasib, wherein R¹ does not include the substituted piperazinyl moiety or equivalent for compounds described in WO 2021/037018.

In embodiments, R¹ is a monovalent form of MRTX1257. In embodiments, R¹ is a monovalent form of a compound as described in US 2018/0072723, which is herein incorporated by reference in its entirety for all purposes. In embodiments, R¹ is a monovalent form of

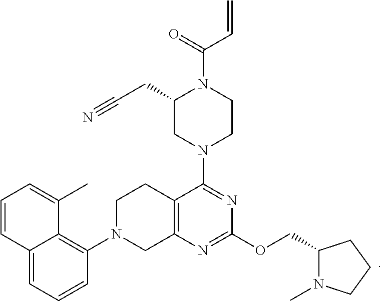 [SMILES: C=CC(=O)N1CCN(c2nc(OC[C@@H]3CCCN3C)nc3c2CCN(c2cccc4cccc(C)c24)C3)C[C@@H]1CC#N]

In embodiments, R¹ is

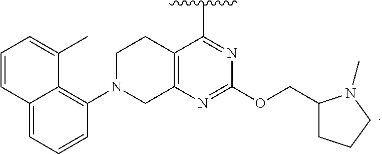 [SMILES: *c1nc(OCC2CCCN2C)nc2c1CCN(c1cccc3cccc(C)c13)C2]

In embodiments, R¹ is

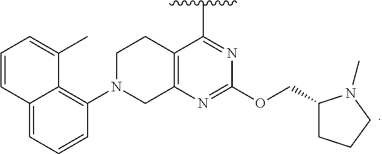 [SMILES: *c1nc(OC[C@H]2CCCN2C)nc2c1CCN(c1cccc3cccc(C)c13)C2]

In embodiments, R¹ is

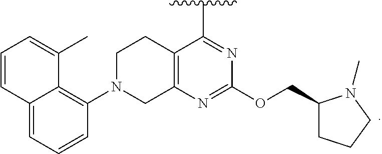 [SMILES: *c1nc(OC[C@@H]2CCCN2C)nc2c1CCN(c1cccc3cccc(C)c13)C2]

In embodiments, R¹ is a monovalent form of a portion of MRTX1257, wherein R¹ does not include the substituted piperazinyl moiety or equivalent for compounds described in US 2018/0072723.

In embodiments, the compound has the formula:

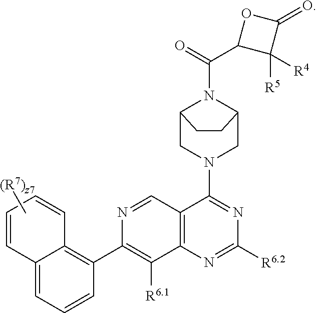

R⁴, R⁵, R⁷, and z7 are as described herein, including in embodiments R^{6.1} and R^{6.2} are independently hydrogen or any value of R⁶ as described herein, including in embodiments.

In embodiments, the compound has the formula:

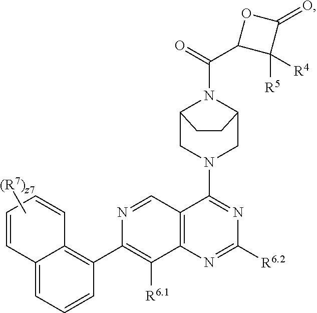

wherein R⁴ and R⁵ are independently hydrogen or unsubstituted C₁-C₄ alkyl; R^{6.1} is halogen; R^{6.2} is —O—(C₁-C₄ alkyl), wherein the C₁-C₄ alkyl is substituted with a 5 to 8 membered heterocycloalkyl optionally substituted with halogen or unsubstituted C₁-C₃ alkyl; R⁷ is independently halogen, —OH, or unsubstituted C₂-C₄ alkynyl (e.g., C₂ alkynyl); and z7 is 1, 2, or 3. In embodiments, R⁴ and R⁵ are independently hydrogen or unsubstituted C₁-C₄ alkyl; R^{6.1} is halogen; R^{6.2} is —O—CH₂-(5 to 8 membered heterocycloalkyl), wherein the 5 to 8 membered heterocycloalkyl is optionally substituted with halogen; R⁷ is independently halogen, —OH, or unsubstituted C₂ alkynyl; and z7 is 1, 2, or 3.

In embodiments, the compound has the formula:

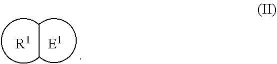

R¹ and E¹ are as described herein, including in embodiments.

In embodiments, the compound has the formula:

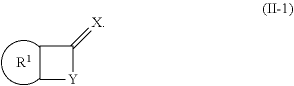

X, Y, and R¹ are as described herein, including in embodiments.

In embodiments, the compound has the formula:

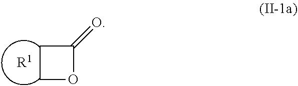

R¹ is as described herein, including in embodiments.

In embodiments, the compound has the formula:

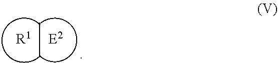

R¹ and E² are as described herein, including in embodiments.

In embodiments, the compound has the formula:

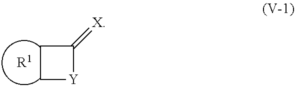

X, Y, and R¹ are as described herein, including in embodiments.

In embodiments, the compound has the formula:

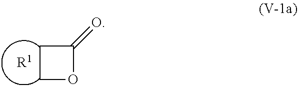

R¹ is as described herein, including in embodiments.

In embodiments, R¹ is

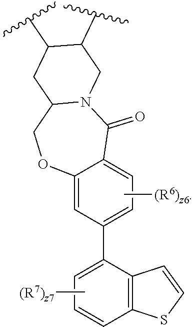

R⁶, z6, R⁷, and z7 are as described herein, including in embodiments.

In embodiments, R¹ is a divalent form of a compound as described in WO2021118877, which is herein incorporated by reference in its entirety for all purposes. In embodiments, R¹ is

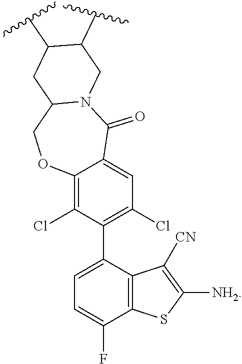 [SMILES: *C1CC2COc3c(cc(Cl)c(-c4ccc(F)c5sc(N)c(C#N)c45)c3Cl)C(=O)N2CC1*]

In embodiments, the compound has the formula:

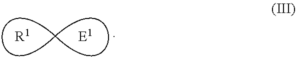

R¹ and E¹ are as described herein, including in embodiments.

In embodiments, the compound has the formula:

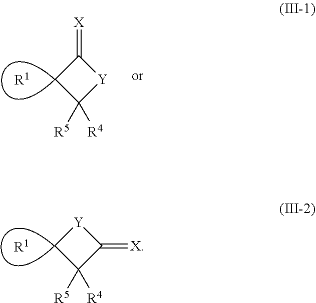

X, Y, R¹, R⁴, and R⁵ are as described herein, including in embodiments. In embodiments, the compound has the formula:

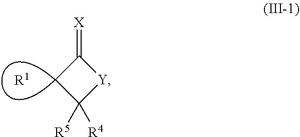

wherein X, Y, R¹, R⁴, and R⁵ are as described herein, including in embodiments. In embodiments, the compound has the formula:

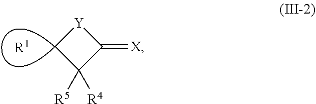

wherein X, Y, R¹, R⁴, and R⁵ are as described herein, including in embodiments.

In embodiments, the compound has the formula:

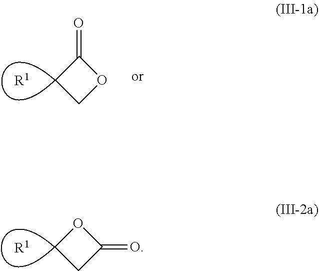

R¹ is as described herein, including in embodiments. In embodiments, the compound has the formula:

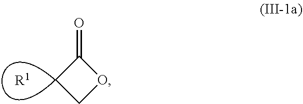

wherein R¹ is as described herein, including in embodiments. In embodiments, the compound has the formula:

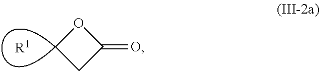

wherein R¹ is as described herein, including in embodiments.

In embodiments, the compound has the formula:

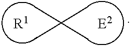

R¹ and E² are as described herein, including in embodiments.

In embodiments, the compound has the formula:

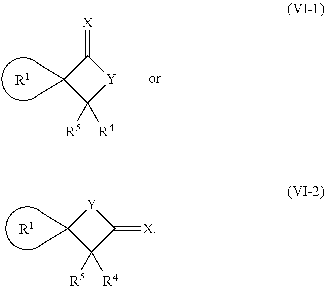

X, Y, R¹, R⁴, and R⁵ are as described herein, including in embodiments. In embodiments, the compound has the formula:

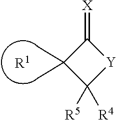

wherein X, Y, R¹, R⁴, and R⁵ are as described herein, including in embodiments. In embodiments, the compound has the formula:

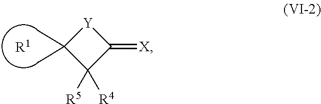

wherein X, Y, R¹, R⁴, and R⁵ are as described herein, including in embodiments.

In embodiments, the compound has the formula:

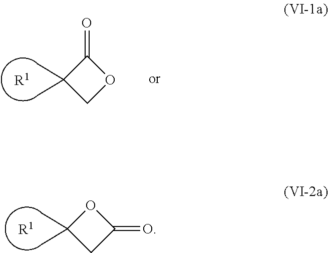

R¹ is as described herein, including in embodiments. In embodiments, the compound has the formula:

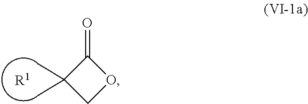

wherein R¹ is as described herein, including in embodiments. In embodiments, the compound has the formula:

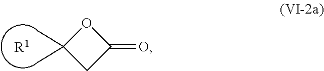

wherein R¹ is as described herein, including in embodiments.

In embodiments, R¹ is

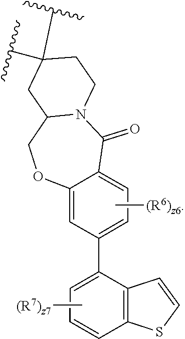

R⁶, z6, R⁷, and z7 are as described herein, including in embodiments.

In embodiments, R¹ is a divalent form of a compound as described in WO2021118877, which is herein incorporated by reference in its entirety for all purposes. In embodiments, R¹ is

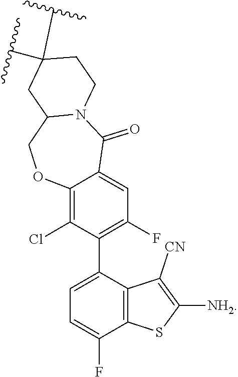 [SMILES: *C1(*)CCN2C(=O)c3cc(F)c(-c4ccc(F)c5sc(N)c(C#N)c45)c(Cl)c3OCC2C1]

In embodiments, when R² is substituted, R² is substituted with one or more first substituent groups denoted by R^{2.1} as explained in the definitions section above in the description of “first substituent group(s)”. In embodiments, when an R²¹ substituent group is substituted, the R^{2.1} substituent group is substituted with one or more second substituent groups denoted by R^{2.2} as explained in the definitions section above in the description of “first substituent group(s)”. In embodiments, when an R^{2.2} substituent group is substituted, the R^{2.2} substituent group is substituted with one or more third substituent groups denoted by R^{2.3} as explained in the definitions section above in the description of “first substituent group(s)”. In the above embodiments, R², R^{2.1}, R^{2.2}, and R^{2.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R², R^{2.1}, R^{2.2}, and R^{2.3}, respectively.

In embodiments, when R³ is substituted, R³ is substituted with one or more first substituent groups denoted by R^{3.1} as explained in the definitions section above in the description of “first substituent group(s)”. In embodiments, when an R^{3.1} substituent group is substituted, the R^{3.1} substituent group is substituted with one or more second substituent groups denoted by R^{3.2} as explained in the definitions section above in the description of “first substituent group(s)”. In embodiments, when an R^{3.2} substituent group is substituted, the R^{3.2} substituent group is substituted with one or more third substituent groups denoted by R^{3.3} as explained in the definitions section above in the description of “first substituent group(s)”. In the above embodiments, R³, R^{3.1}, R^{3.2}, and R^{3.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R³, R^{3.1}, R^{3.2}, and R^{3.3}, respectively.

In embodiments, when two R³ substituents are optionally joined to form a moiety that is substituted (e.g., a substituted cycloalkyl, substituted heterocycloalkyl, substituted aryl, or substituted heteroaryl), the moiety is substituted with one or more first substituent groups denoted by R^{3.1} as explained in the definitions section above in the description of “first substituent group(s)”. In embodiments, when an R^{3.1} substituent group is substituted, the R^{3.1} substituent group is substituted with one or more second substituent groups denoted by R^{3.2} as explained in the definitions section above in the description of “first substituent group(s)”. In embodiments, when an R^{3.2} substituent group is substituted, the R^{3.2} substituent group is substituted with one or more third substituent groups denoted by R^{3.3} as explained in the definitions section above in the description of “first substituent group(s)”. In the above embodiments, R³, R^{3.1}, R^{3.2}, and R^{3.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R³, R^{3.1}, R^{3.2}, and R^{3.3}, respectively.

In embodiments, when R⁴ is substituted, R⁴ is substituted with one or more first substituent groups denoted by R^{4.1} as explained in the definitions section above in the description of “first substituent group(s)”. In embodiments, when an R^{4.1} substituent group is substituted, the R^{4.1} substituent group is substituted with one or more second substituent groups denoted by R^{4.2} as explained in the definitions section above in the description of “first substituent group(s)”. In embodiments, when an R^{4.2} substituent group is substituted, the R^{4.2} substituent group is substituted with one or more third substituent groups denoted by R^{4.3} as explained in the definitions section above in the description of “first substituent group(s)”. In the above embodiments, R⁴, R^{4.1}, R^{4.2}, and R^{4.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R⁴, R^{4.1}, R^{4.2}, and R⁴-3, respectively.

In embodiments, when R⁵ is substituted, R⁵ is substituted with one or more first substituent groups denoted by R^{5.1} as explained in the definitions section above in the description of “first substituent group(s)”. In embodiments, when an R^{5.1} substituent group is substituted, the R^{5.1} substituent group is substituted with one or more second substituent groups denoted by R^{5.2} as explained in the definitions section above in the description of “first substituent group(s)”. In embodiments, when an R^{5.2} substituent group is substituted, the R^{5.2} substituent group is substituted with one or more third substituent groups denoted by R⁵ as explained in the definitions section above in the description of “first substituent group(s)”. In the above embodiments, R⁵, R^{5.1}, R^{5.2}, and R⁵³ have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R⁵, R^{5.1}, R⁵0.2, and R⁵³, respectively.

In embodiments, when R⁶ is substituted, R⁶ is substituted with one or more first substituent groups denoted by R^{6.1} as explained in the definitions section above in the description of “first substituent group(s)”. In embodiments, when an R^{6.1} substituent group is substituted, the R^{6.1} substituent group is substituted with one or more second substituent groups denoted by R^{6.2} as explained in the definitions section above in the description of “first substituent group(s)”. In embodiments, when an R^{6.2} substituent group is substituted, the R^{6.2} substituent group is substituted with one or more third substituent groups denoted by R^{6.3} as explained in the definitions section above in the description of “first substituent group(s)”. In the above embodiments, R⁶, R^{6.1}, R^{6.2}, and R^{6.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R⁶, R^{6.1}, R^{6.2}, and R^{6.3}, respectively.

In embodiments, when R⁷ is substituted, R⁷ is substituted with one or more first substituent groups denoted by R⁷¹ as explained in the definitions section above in the description of “first substituent group(s)”. In embodiments, when an R⁷¹ substituent group is substituted, the R⁷¹ substituent group is substituted with one or more second substituent groups denoted by R^{7.2} as explained in the definitions section above in the description of “first substituent group(s)”. In embodiments, when an R^{7.2} substituent group is substituted, the R^{7.2} substituent group is substituted with one or more third substituent groups denoted by R^{7.3} as explained in the definitions section above in the description of “first substituent group(s)”. In the above embodiments, R⁷, R^{7.1}, R^{7.2}, and R^{7.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R⁷, R^{7.1}, R^{7.2}, and R^{7.3}, respectively.

In embodiments, when R⁸ is substituted, R⁸ is substituted with one or more first substituent groups denoted by R^{8.1} as explained in the definitions section above in the description of “first substituent group(s)”. In embodiments, when an R^{8.1} substituent group is substituted, the R^{8.1} substituent group is substituted with one or more second substituent groups denoted by R^{8.2} as explained in the definitions section above in the description of “first substituent group(s)”. In embodiments, when an R^{8.2} substituent group is substituted, the R^{8.2} substituent group is substituted with one or more third substituent groups denoted by R^{8.3} as explained in the definitions section above in the description of “first substituent group(s)”. In the above embodiments, R⁸, R^{8.1}, R^{8.2}, and R^{8.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R⁸, R^{8.1}, R^{8.2}, and R^{8.3}, respectively.

In embodiments, when R⁹ is substituted, R⁹ is substituted with one or more first substituent groups denoted by R^{9.1} as explained in the definitions section above in the description of “first substituent group(s)”. In embodiments, when an R^{9.1} substituent group is substituted, the R^{9.1} substituent group is substituted with one or more second substituent groups denoted by R^{9.2} as explained in the definitions section above in the description of “first substituent group(s)”. In embodiments, when an R^{9.2} substituent group is substituted, the R^{9.2} substituent group is substituted with one or more third substituent groups denoted by R^{9.3} as explained in the definitions section above in the description of “first substituent group(s)”. In the above embodiments, R⁹, R^{9.1}, R^{9.2}, and R^{9.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R⁹, R^{9.1}, R^{9.2}, and R^{9.3}, respectively.

In embodiments, when R¹⁰ is substituted, R¹⁰ is substituted with one or more first substituent groups denoted by R¹⁰¹ as explained in the definitions section above in the description of “first substituent group(s)”. In embodiments, when an R¹⁰ substituent group is substituted, the R¹⁰¹ substituent group is substituted with one or more second substituent groups denoted by R^{10.2} as explained in the definitions section above in the description of “first substituent group(s)”. In embodiments, when an R^{10.2} substituent group is substituted, the R^{10.2} substituent group is substituted with one or more third substituent groups denoted by R^{10.3} as explained in the definitions section above in the description of “first substituent group(s)”. In the above embodiments, R¹⁰, R^{10.1}, R^{10.2}, and R^{10.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R¹⁰, R¹⁰, R^{10.2}, and R¹⁰³, respectively.

In embodiments, when R²⁰ is substituted, R²⁰ is substituted with one or more first substituent groups denoted by R^{20.1} as explained in the definitions section above in the description of “first substituent group(s)”. In embodiments, when an R^{20.1} substituent group is substituted, the R^{20.1} substituent group is substituted with one or more second substituent groups denoted by R^{20.2} as explained in the definitions section above in the description of “first substituent group(s)”. In embodiments, when an R^{20.2} substituent group is substituted, the R^{20.2} substituent group is substituted with one or more third substituent groups denoted by R^{20.3} as explained in the definitions section above in the description of “first substituent group(s)”. In the above embodiments, R²⁰, R^{20.1}, R^{20.2}, and R^{20.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R²⁰, R^{20.1}, R^{20.2}, and R^{20.3}, respectively.

In embodiments, when R^{20A} is substituted, R^{20A} is substituted with one or more first substituent groups denoted by R^{20A.1} as explained in the definitions section above in the description of “first substituent group(s)”. In embodiments, when an R^{20A.1} substituent group is substituted, the R^{20A.1} substituent group is substituted with one or more second substituent groups denoted by R^{20A.2} as explained in the definitions section above in the description of “first substituent group(s)”. In embodiments, when an R^{20A.2} substituent group is substituted, the R^{20A.2} substituent group is substituted with one or more third substituent groups denoted by R^{20A.3} as explained in the definitions section above in the description of “first substituent group(s)”. In the above embodiments, R^{20A}, R^{20A.1}, R^{20A.2}, and R^{20A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{20A}, R^{20A.1}, R^{20A.2} and R^{20A.3} respectively.

In embodiments, when R^{20B} is substituted, R^{20B} is substituted with one or more first substituent groups denoted by R^{20B.1} as explained in the definitions section above in the description of “first substituent group(s)”. In embodiments, when an R^{20B.1} substituent group is substituted, the R^{20B.1} substituent group is substituted with one or more second substituent groups denoted by R^{20B.2} as explained in the definitions section above in the description of “first substituent group(s)”. In embodiments, when an R^{20B.2} substituent group is substituted, the R^{20B.2} substituent group is substituted with one or more third substituent groups denoted by R^{20B.3} as explained in the definitions section above in the description of “first substituent group(s)”. In the above embodiments, R^{20B}, R^{20B.1}, R^{20B.2}, and R^{20B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{20B}, R^{20B.1}, R^{20B.2}, and R^{20B.3}, respectively.

In embodiments, when R^{20A} and R^{20B} substituents bonded to the same nitrogen atom are optionally joined to form a moiety that is substituted (e.g., a substituted heterocycloalkyl or substituted heteroaryl), the moiety is substituted with one or more first substituent groups denoted by R^{20A.1} as explained in the definitions section above in the description of “first substituent group(s)”. In embodiments, when an R^{20A.1} substituent group is substituted, the R^{20A.1} substituent group is substituted with one or more second substituent groups denoted by R^{20A.2} as explained in the definitions section above in the description of “first substituent group(s)”. In embodiments, when an R^{20A.2} substituent group is substituted, the R^{20A.2} substituent group is substituted with one or more third substituent groups denoted by R^{20A.3} as explained in the definitions section above in the description of “first substituent group(s)”. In the above embodiments, R^{20A.1}, R^{20A.2}, and R^{20A.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{20A.1}, R^{20A.2}, and R^{20A.3}, respectively.

In embodiments, when R^{20A} and R^{20B} substituents bonded to the same nitrogen atom are optionally joined to form a moiety that is substituted (e.g., a substituted heterocycloalkyl or substituted heteroaryl), the moiety is substituted with one or more first substituent groups denoted by R^{20B.1} as explained in the definitions section above in the description of “first substituent group(s)”. In embodiments, when an R^{20B.1} substituent group is substituted, the R^{20B.1} substituent group is substituted with one or more second substituent groups denoted by R^{20B.2} as explained in the definitions section above in the description of “first substituent group(s)”. In embodiments, when an R^{20B.2} substituent group is substituted, the R^{20B.2} substituent group is substituted with one or more third substituent groups denoted by R^{20B.3} as explained in the definitions section above in the description of “first substituent group(s)”. In the above embodiments, R^{20B.1}, R^{20B.2}, and R^{20B.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{20B.1}, R^{20B.2}, and R^{20B.3}, respectively.

In embodiments, when R^{20C} is substituted, R^{20C} is substituted with one or more first substituent groups denoted by R^{20C.1} as explained in the definitions section above in the description of “first substituent group(s)”. In embodiments, when an R^{20C.1} substituent group is substituted, the R^{20C.1} substituent group is substituted with one or more second substituent groups denoted by R^{20C.2} as explained in the definitions section above in the description of “first substituent group(s)”. In embodiments, when an R^{20C.2} substituent group is substituted, the R^{20C.2} substituent group is substituted with one or more third substituent groups denoted by R^{20C.3} as explained in the definitions section above in the description of “first substituent group(s)”. In the above embodiments, R^{20C}, R^{20C.1}, R^{20C.2}, and R^{20C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{20C}, R^{20C.1}, R^{20C.2} and R^{20C.3}, respectively.

In embodiments, when R^{20D} is substituted, R^{20D} is substituted with one or more first substituent groups denoted by R^{20D.1} as explained in the definitions section above in the description of “first substituent group(s)”. In embodiments, when an R^{20D.1} substituent group is substituted, the R^{20D.1} substituent group is substituted with one or more second substituent groups denoted by R^{20D.2} as explained in the definitions section above in the description of “first substituent group(s)”. In embodiments, when an R^{20D.2} substituent group is substituted, the R^{20D.2} substituent group is substituted with one or more third substituent groups denoted by R^{20D.3} as explained in the definitions section above in the description of “first substituent group(s)”. In the above embodiments, R^{20D}, R^{20D.1}, R^{20D.2}, and R^{20D.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{20D}, R^{20D.1}, R^{20D.2} and R^{20D.3} respectively.

In embodiments, when R¹⁰¹ is substituted, R¹⁰¹ is substituted with one or more first substituent groups denoted by R^{101.1} as explained in the definitions section above in the description of “first substituent group(s)”. In embodiments, when an R^{101.1} substituent group is substituted, the R^{101.1} substituent group is substituted with one or more second substituent groups denoted by R^{101.2} as explained in the definitions section above in the description of “first substituent group(s)”. In embodiments, when an R^{101.2} substituent group is substituted, the R^{101.2} substituent group is substituted with one or more third substituent groups denoted by R^{101.3} as explained in the definitions section above in the description of “first substituent group(s)”. In the above embodiments, R¹⁰¹, R^{101.1}, R^{101.2}, and R^{101.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R¹⁰¹, R^{101.1}, R^{101.2} and R^{101.3} respectively.

In embodiments, when R¹⁰² is substituted, R¹⁰² is substituted with one or more first substituent groups denoted by R^{102.1} as explained in the definitions section above in the description of “first substituent group(s)”. In embodiments, when an R^{102.1} substituent group is substituted, the R^{102.1} substituent group is substituted with one or more second substituent groups denoted by R^{102.2} as explained in the definitions section above in the description of “first substituent group(s)”. In embodiments, when an R^{102.2} substituent group is substituted, the R^{102.2} substituent group is substituted with one or more third substituent groups denoted by R^{102.3} as explained in the definitions section above in the description of “first substituent group(s)”. In the above embodiments, R¹⁰², R^{102.1}, R^{102.2}, and R^{102.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R¹⁰², R^{102.1}, R^{102.2} and R^{102.3}, respectively.

In embodiments, when R¹⁰³ is substituted, R¹⁰³ is substituted with one or more first substituent groups denoted by R^{13.1} as explained in the definitions section above in the description of “first substituent group(s)”. In embodiments, when an R^{13.1} substituent group is substituted, the R^{103.1} substituent group is substituted with one or more second substituent groups denoted by R^{103.2} as explained in the definitions section above in the description of “first substituent group(s)”. In embodiments, when an R^{103.2} substituent group is substituted, the R^{103.2} substituent group is substituted with one or more third substituent groups denoted by R^{103.3} as explained in the definitions section above in the description of “first substituent group(s)”. In the above embodiments, R¹⁰³, R^{103.1}, R^{103.2}, and R^{103.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R¹⁰³, R^{103.1}, R^{103.2} and R^{103.3} respectively.

In embodiments, when R²⁰⁰ is substituted, R²⁰⁰ is substituted with one or more first substituent groups denoted by R^{200.1} as explained in the definitions section above in the description of “first substituent group(s)”. In embodiments, when an R^{200.1} substituent group is substituted, the R^{200.1} substituent group is substituted with one or more second substituent groups denoted by R^{200.2} as explained in the definitions section above in the description of “first substituent group(s)”. In embodiments, when an R^{200.2} substituent group is substituted, the R^{200.2} substituent group is substituted with one or more third substituent groups denoted by R^{200.3} as explained in the definitions section above in the description of “first substituent group(s)”. In the above embodiments, R²⁰⁰, R^{200.1}, R^{200.2}, and R^{200.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R²⁰⁰, R^{200.1}, R^{200.2}, and R^{200.3}, respectively.

In embodiments, when L²⁰ is substituted, L²⁰ is substituted with one or more first substituent groups denoted by R^{L20.1} as explained in the definitions section above in the description of “first substituent group(s)”. In embodiments, when an R^{L20.1} substituent group is substituted, the R^{L20.1} substituent group is substituted with one or more second substituent groups denoted by R^{L20.2} as explained in the definitions section above in the description of “first substituent group(s)”. In embodiments, when an R^{L20.2} substituent group is substituted, the R^{L20.2} substituent group is substituted with one or more third substituent groups denoted by R^{L20.3} as explained in the definitions section above in the description of “first substituent group(s)”. In the above embodiments, L²⁰, R^{L20.1}, R^{L20.2}, and R^{L20.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L²⁰, R^{L20.1}, R^{L20.2}, and R^{L20.3}, respectively.

In embodiments, when L¹⁰¹ is substituted, L¹⁰¹ is substituted with one or more first substituent groups denoted by R^{L101.1} as explained in the definitions section above in the description of “first substituent group(s)”. In embodiments, when an R^{L101.1} substituent group is substituted, the R^{L101.1} substituent group is substituted with one or more second substituent groups denoted by R^{L101.2} as explained in the definitions section above in the description of “first substituent group(s)”. In embodiments, when an R^{L101.2} substituent group is substituted, the R^{L101.2} substituent group is substituted with one or more third substituent groups denoted by R^{L101.3} as explained in the definitions section above in the description of “first substituent group(s)”. In the above embodiments, L¹⁰¹, R^{L101.1}, R^{L101.2}, and R^{L101.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L¹⁰¹, R^{L101.1}, R^{L101.2}, and R^{L101.3}, respectively.

In embodiments, when L¹⁰² is substituted, L¹⁰² is substituted with one or more first substituent groups denoted by R^{L102.1} as explained in the definitions section above in the description of “first substituent group(s)”. In embodiments, when an R^{L102.1} substituent group is substituted, the R^{L102.1} substituent group is substituted with one or more second substituent groups denoted by R^{L102.2} as explained in the definitions section above in the description of “first substituent group(s)”. In embodiments, when an R^{L102.2} substituent group is substituted, the R^{L102.2} substituent group is substituted with one or more third substituent groups denoted by R^{L102.3} as explained in the definitions section above in the description of “first substituent group(s)”. In the above embodiments, L¹⁰², R^{L102.1}, R^{L102.2}, and R^{L102.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L¹⁰², R^{L102.1}, R^{L102.2}, and R^{L102.3}, respectively.

In embodiments, when L¹⁰³ is substituted, L¹⁰³ is substituted with one or more first substituent groups denoted by R^{L103.1} as explained in the definitions section above in the description of “first substituent group(s)”. In embodiments, when an R^{L103.1} substituent group is substituted, the R^{L103.1} substituent group is substituted with one or more second substituent groups denoted by R^{L103.2} as explained in the definitions section above in the description of “first substituent group(s)”. In embodiments, when an R^{L103.2} substituent group is substituted, the R^{L103.2} substituent group is substituted with one or more third substituent groups denoted by R^{L103.3} as explained in the definitions section above in the description of “first substituent group(s)”. In the above embodiments, L¹⁰³, R^{L103.1}, R^{L103.2}, and R^{L103.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L¹⁰³, R^{L103.1}, R^{L103.2}, and R^{L103.3}, respectively.

In embodiments, the compound has the formula:

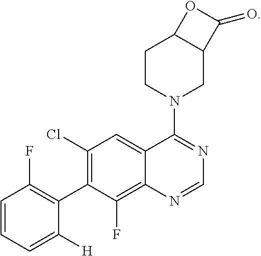 [SMILES: O=C1OC2CCN(c3ncnc4c(F)c(-c5ccccc5F)c(Cl)cc34)CC12]

In embodiments, the compound has the formula:

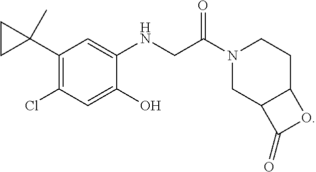 [SMILES: CC1(c2cc(NCC(=O)N3CCC4OC(=O)C4C3)c(O)cc2Cl)CC1]

In embodiments, the compound has the formula:

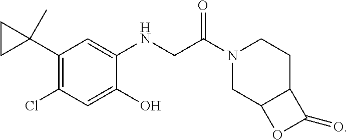 [SMILES: CC1(c2cc(NCC(=O)N3CCC4C(=O)OC4C3)c(O)cc2Cl)CC1]

In embodiments, the compound has the formula:

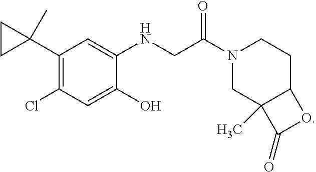 [SMILES: CC1(c2cc(NCC(=O)N3CCC4OC(=O)C4(C)C3)c(O)cc2Cl)CC1]

In embodiments, the compound has the formula:

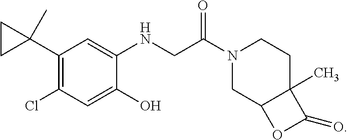 [SMILES: CC1(c2cc(NCC(=O)N3CCC4(C)C(=O)OC4C3)c(O)cc2Cl)CC1]

In embodiments, the compound has the formula:

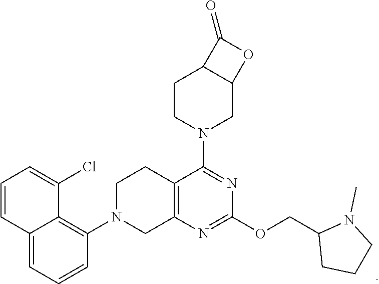 [SMILES: CN1CCCC1COc1nc2c(c(N3CCC4C(=O)OC4C3)n1)CCN(c1cccc3cccc(Cl)c13)C2]

In embodiments, the compound has the formula.

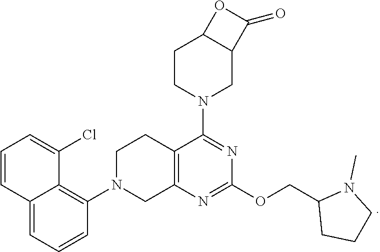 [SMILES: CN1CCCC1COc1nc2c(c(N3CCC4OC(=O)C4C3)n1)CCN(c1cccc3cccc(Cl)c13)C2]

In embodiments, the compound has the formula:

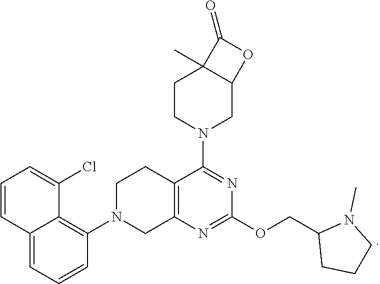 [SMILES: CN1CCCC1COc1nc2c(c(N3CCC4(C)C(=O)OC4C3)n1)CCN(c1cccc3cccc(Cl)c13)C2]

In embodiments, the compound has the formula:

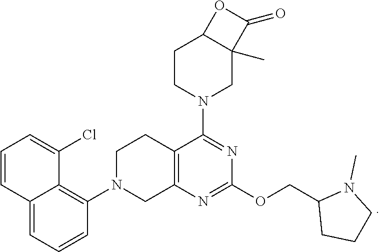 [SMILES: CN1CCCC1COc1nc2c(c(N3CCC4OC(=O)C4(C)C3)n1)CCN(c1cccc3cccc(Cl)c13)C2]

In embodiments, the compound has the formula:

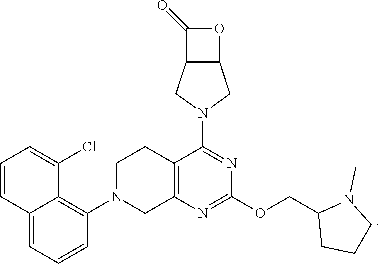 [SMILES: CN1CCCC1COc1nc2c(c(N3CC4OC(=O)C4C3)n1)CCN(c1cccc3cccc(Cl)c13)C2]

In embodiments, the compound has the formula:

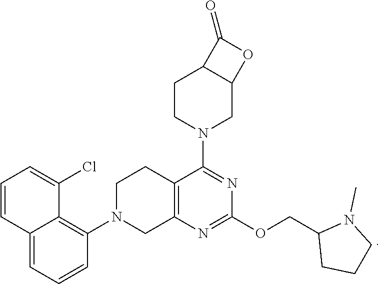 [SMILES: CN1CCCC1COc1nc2c(c(N3CCC4C(=O)OC4C3)n1)CCN(c1cccc3cccc(Cl)c13)C2]

In embodiments, the compound has the formula:

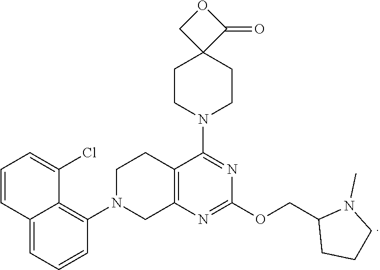 [SMILES: CN1CCCC1COc1nc2c(c(N3CCC4(CC3)COC4=O)n1)CCN(c1cccc3cccc(Cl)c13)C2]

In embodiments, the compound has the formula:

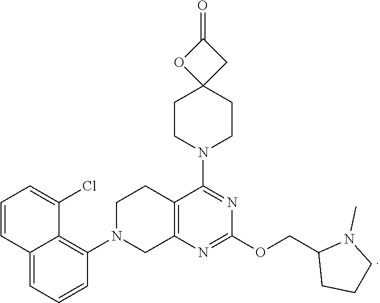 [SMILES: CN1CCCC1COc1nc2c(c(N3CCC4(CC3)CC(=O)O4)n1)CCN(c1cccc3cccc(Cl)c13)C2]

In embodiments, the compound has the formula:

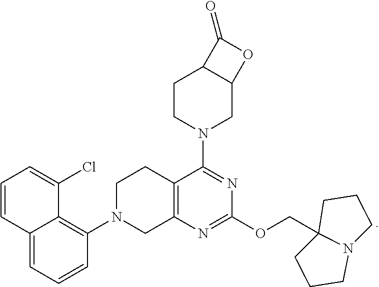 [SMILES: O=C1OC2CN(c3nc(OCC45CCCN4CCC5)nc4c3CCN(c3cccc5cccc(Cl)c35)C4)CCC12]

In embodiments, the compound has the formula:

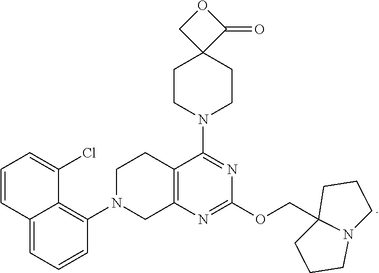 [SMILES: O=C1OCC12CCN(c1nc(OCC34CCCN3CCC4)nc3c1CCN(c1cccc4cccc(Cl)c14)C3)CC2]

In embodiments, the compound has the formula:

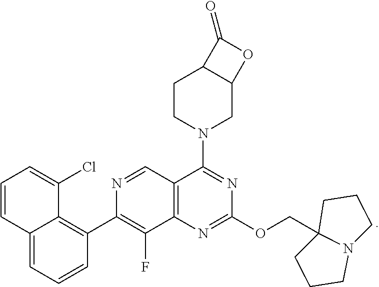 [SMILES: O=C1OC2CN(c3nc(OCC45CCCN4CCC5)nc4c(F)c(-c5cccc6cccc(Cl)c56)ncc34)CCC12]

In embodiments, the compound has the formula:

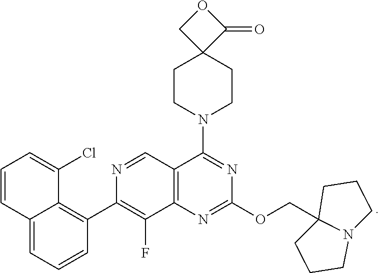 [SMILES: O=C1OCC12CCN(c1nc(OCC34CCCN3CCC4)nc3c(F)c(-c4cccc5cccc(Cl)c45)ncc13)CC2]

In embodiments, the compound has the formula:

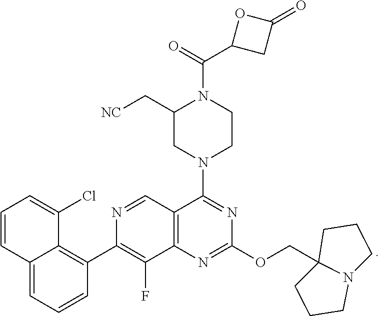 [SMILES: N#CCC1CN(c2nc(OCC34CCCN3CCC4)nc3c(F)c(-c4cccc5cccc(Cl)c45)ncc23)CCN1C(=O)C1CC(=O)O1]

In embodiments, the compound has the formula:

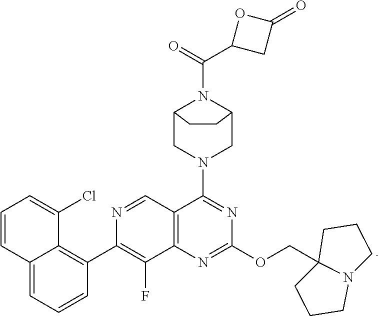 [SMILES: O=C1CC(C(=O)N2C3CCC2CN(c2nc(OCC45CCCN4CCC5)nc4c(F)c(-c5cccc6cccc(Cl)c56)ncc24)C3)O1]

In embodiments, the compound has the formula.

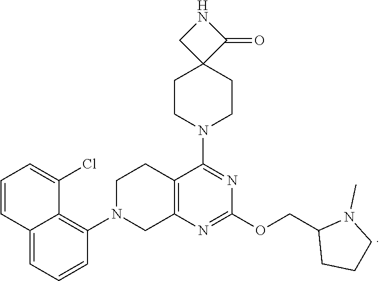 [SMILES: CN1CCCC1COc1nc2c(c(N3CCC4(CC3)CNC4=O)n1)CCN(c1cccc3cccc(Cl)c13)C2]

In embodiments, the compound has the formula:

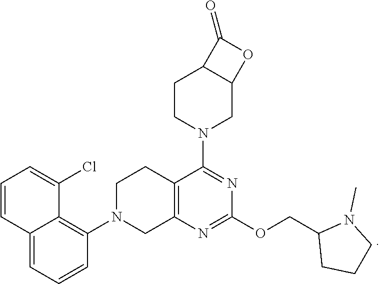 [SMILES: CN1CCCC1COc1nc2c(c(N3CCC4C(=O)OC4C3)n1)CCN(c1cccc3cccc(Cl)c13)C2]

In embodiments, the compound has the formula:

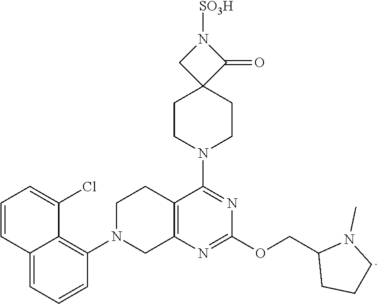 [SMILES: CN1CCCC1COc1nc2c(c(N3CCC4(CC3)CN(S(=O)(=O)O)C4=O)n1)CCN(c1cccc3cccc(Cl)c13)C2]

In embodiments, the compound has the formula:

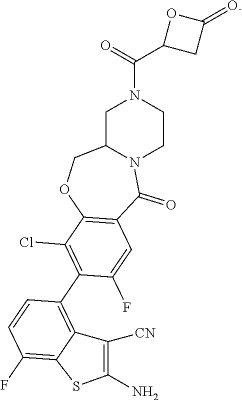 [SMILES: N#Cc1c(N)sc2c(F)ccc(-c3c(F)cc4c(c3Cl)OCC3CN(C(=O)C5CC(=O)O5)CCN3C4=O)c12]

In embodiments, the compound has the formula:

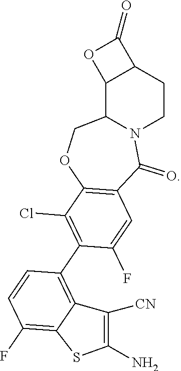 [SMILES: N#Cc1c(N)sc2c(F)ccc(-c3c(F)cc4c(c3Cl)OCC3C5OC(=O)C5CCN3C4=O)c12]

In embodiments, the compound has the formula:

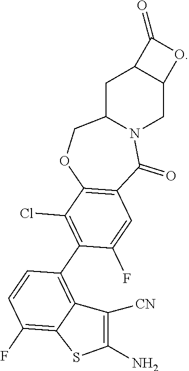 [SMILES: N#Cc1c(N)sc2c(F)ccc(-c3c(F)cc4c(c3Cl)OCC3CC5C(=O)OC5CN3C4=O)c12]

In embodiments, the compound has the formula:

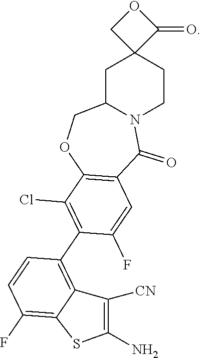 [SMILES: N#Cc1c(N)sc2c(F)ccc(-c3c(F)cc4c(c3Cl)OCC3CC5(CCN3C4=O)COC5=O)c12]

In embodiments, the compound has the formula:

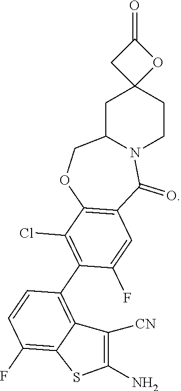 [SMILES: N#Cc1c(N)sc2c(F)ccc(-c3c(F)cc4c(c3Cl)OCC3CC5(CCN3C4=O)CC(=O)O5)c12]

In embodiments, the compound has the formula:

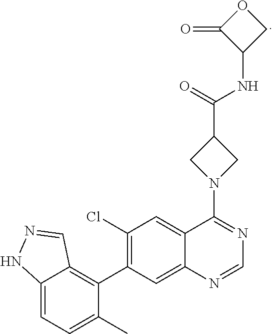 [SMILES: Cc1ccc2[nH]ncc2c1-c1cc2ncnc(N3CC(C(=O)NC4COC4=O)C3)c2cc1Cl]

In embodiments, the compound has the formula:

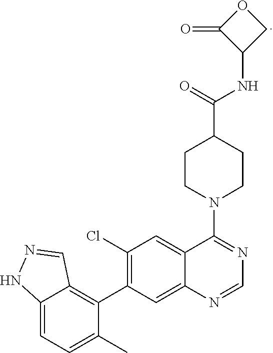 [SMILES: Cc1ccc2[nH]ncc2c1-c1cc2ncnc(N3CCC(C(=O)NC4COC4=O)CC3)c2cc1Cl]

In embodiments, the compound has the formula:

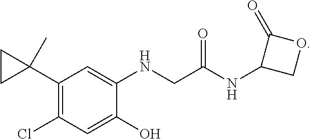 [SMILES: CC1(c2cc(NCC(=O)NC3COC3=O)c(O)cc2Cl)CC1]

In embodiments, the compound has the formula:

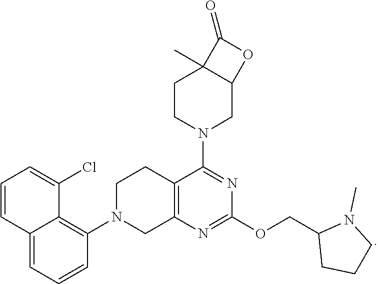 [SMILES: CN1CCCC1COc1nc2c(c(N3CCC4(C)C(=O)OC4C3)n1)CCN(c1cccc3cccc(Cl)c13)C2]

In embodiments, the compound has the formula:

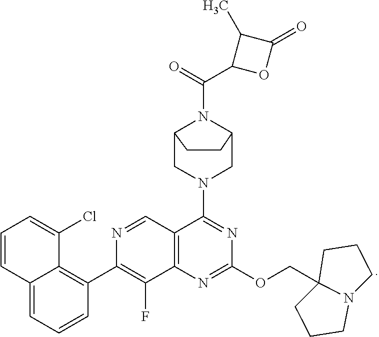 [SMILES: CC1C(=O)OC1C(=O)N1C2CCC1CN(c1nc(OCC34CCCN3CCC4)nc3c(F)c(-c4cccc5cccc(Cl)c45)ncc13)C2]

In embodiments, the compound has the formula:

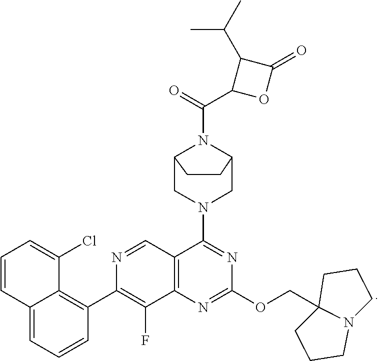 [SMILES: CC(C)C1C(=O)OC1C(=O)N1C2CCC1CN(c1nc(OCC34CCCN3CCC4)nc3c(F)c(-c4cccc5cccc(Cl)c45)ncc13)C2]

In embodiments, the compound has the formula:

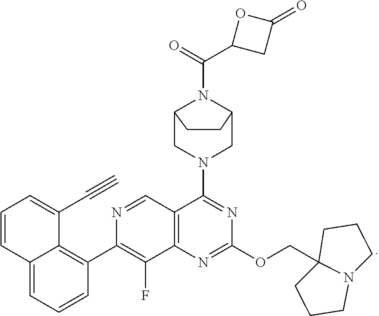 [SMILES: C#Cc1cccc2cccc(-c3ncc4c(N5CC6CCC(C5)N6C(=O)C5CC(=O)O5)nc(OCC56CCCN5CCC6)nc4c3F)c12]

In embodiments, the compound has the formula:

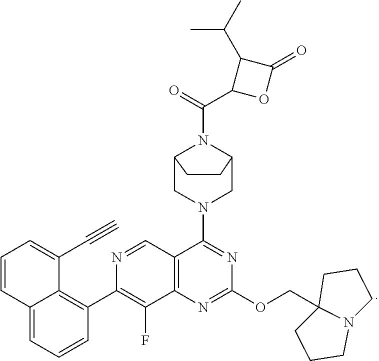 [SMILES: C#Cc1cccc2cccc(-c3ncc4c(N5CC6CCC(C5)N6C(=O)C5OC(=O)C5C(C)C)nc(OCC56CCCN5CCC6)nc4c3F)c12]

In embodiments, the compound has the formula:

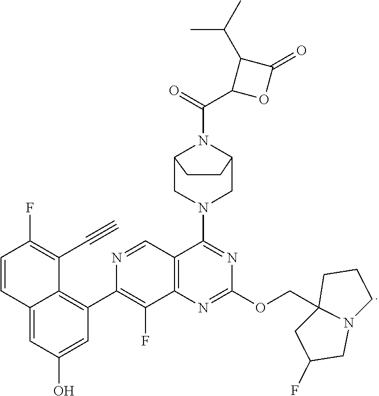 [SMILES: C#Cc1c(F)ccc2cc(O)cc(-c3ncc4c(N5CC6CCC(C5)N6C(=O)C5OC(=O)C5C(C)C)nc(OCC56CCCN5CC(F)C6)nc4c3F)c12]

In embodiments, the compound has the formula:

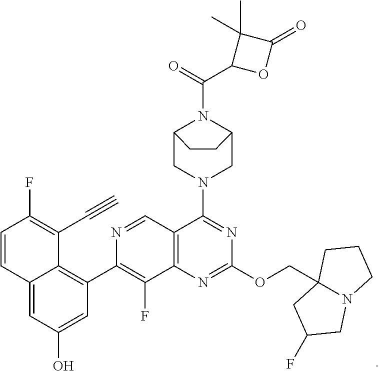 [SMILES: C#Cc1c(F)ccc2cc(O)cc(-c3ncc4c(N5CC6CCC(C5)N6C(=O)C5OC(=O)C5(C)C)nc(OCC56CCCN5CC(F)C6)nc4c3F)c12]

In embodiments, the compound has the formula:

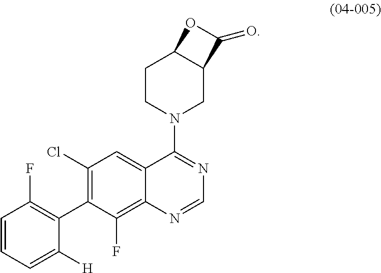 [SMILES: O=C1O[C@@H]2CCN(c3ncnc4c(F)c(-c5ccccc5F)c(Cl)cc34)C[C@H]12]

In embodiments, the compound has the formula:

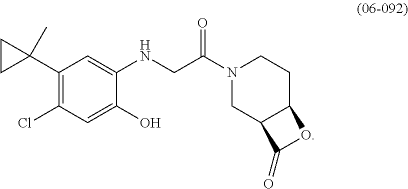 [SMILES: CC1(c2cc(NCC(=O)N3CC[C@H]4OC(=O)[C@H]4C3)c(O)cc2Cl)CC1]

In embodiments, the compound has the formula:

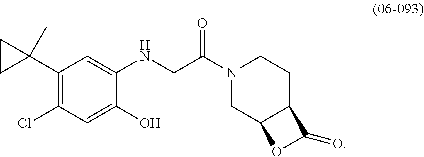 [SMILES: CC1(c2cc(NCC(=O)N3CC[C@H]4C(=O)O[C@H]4C3)c(O)cc2Cl)CC1]

In embodiments, the compound has the formula:

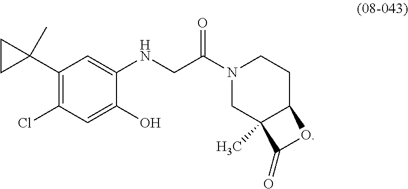 [SMILES: CC1(c2cc(NCC(=O)N3CC[C@H]4OC(=O)[C@@]4(C)C3)c(O)cc2Cl)CC1]

In embodiments, the compound has the formula:

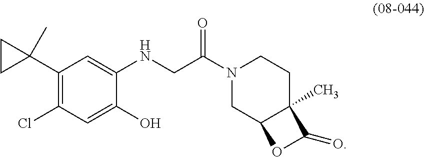 [SMILES: CC1(c2cc(NCC(=O)N3CC[C@@]4(C)C(=O)O[C@H]4C3)c(O)cc2Cl)CC1]

In embodiments, the compound has the formula:

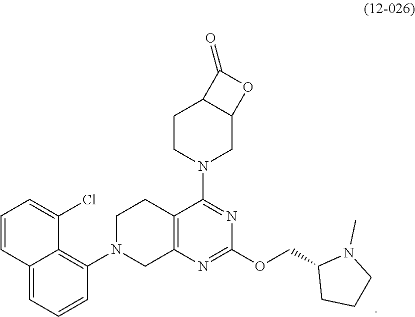 [SMILES: CN1CCC[C@@H]1COc1nc2c(c(N3CCC4C(=O)OC4C3)n1)CCN(c1cccc3cccc(Cl)c13)C2]

In embodiments, the compound has the formula:

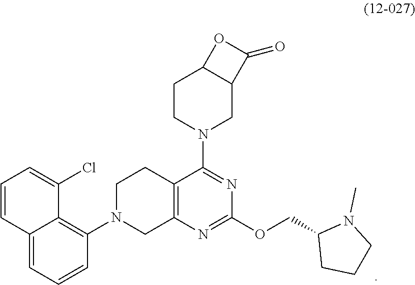 [SMILES: CN1CCC[C@@H]1COc1nc2c(c(N3CCC4OC(=O)C4C3)n1)CCN(c1cccc3cccc(Cl)c13)C2]

In embodiments, the compound has the formula:

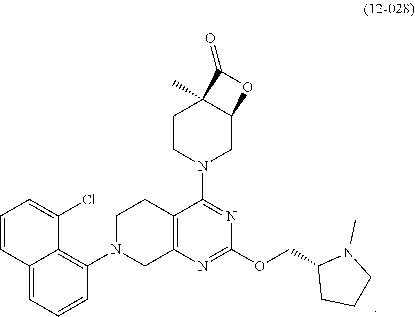 [SMILES: CN1CCC[C@@H]1COc1nc2c(c(N3CC[C@@]4(C)C(=O)O[C@H]4C3)n1)CCN(c1cccc3cccc(Cl)c13)C2]

In embodiments, the compound has the formula:

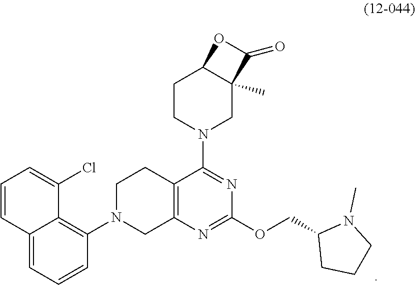 [SMILES: CN1CCC[C@@H]1COc1nc2c(c(N3CC[C@H]4OC(=O)[C@@]4(C)C3)n1)CCN(c1cccc3cccc(Cl)c13)C2]

In embodiments, the compound has the formula:

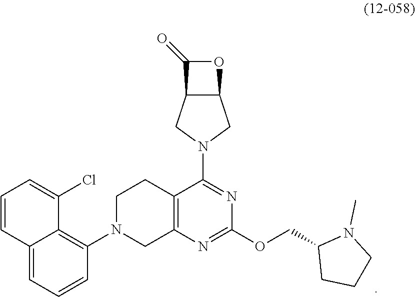 [SMILES: CN1CCC[C@@H]1COc1nc2c(c(N3C[C@@H]4OC(=O)[C@@H]4C3)n1)CCN(c1cccc3cccc(Cl)c13)C2]

In embodiments, the compound has the formula:

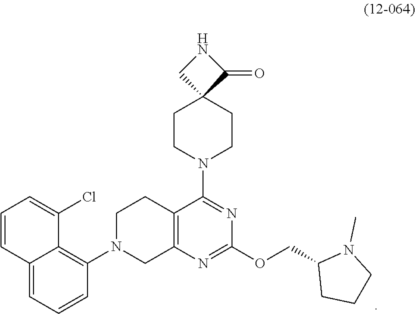

In embodiments, the compound has the formula:

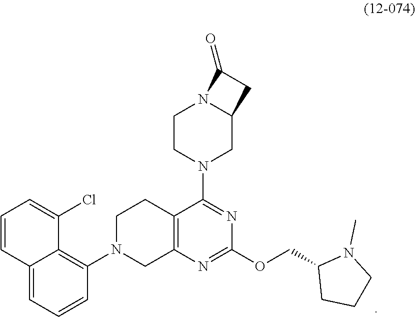

In embodiments, the compound has the formula:

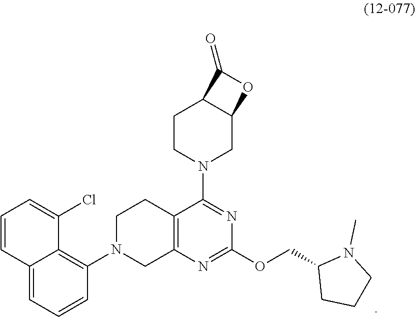 [SMILES: CN1CCC[C@@H]1COc1nc2c(c(N3CC[C@H]4C(=O)O[C@H]4C3)n1)CCN(c1cccc3cccc(Cl)c13)C2]

In embodiments, the compound has the formula:

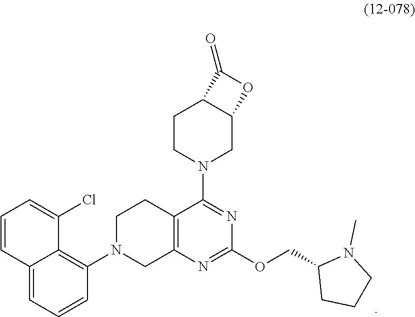 [SMILES: CN1CCC[C@@H]1COc1nc2c(c(N3CC[C@@H]4C(=O)O[C@@H]4C3)n1)CCN(c1cccc3cccc(Cl)c13)C2]

In embodiments, the compound has the formula:

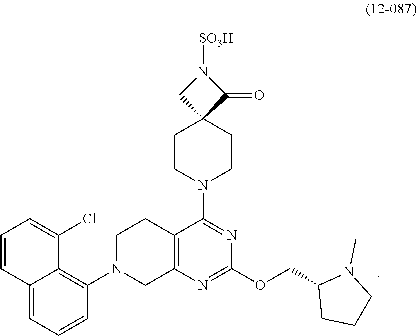

In embodiments, the compound has the formula:

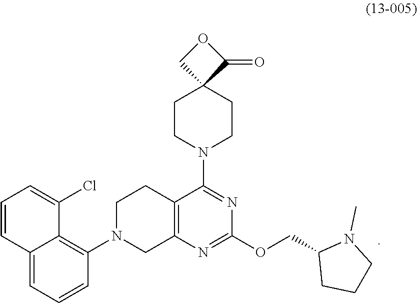

In embodiments, the compound has the formula:

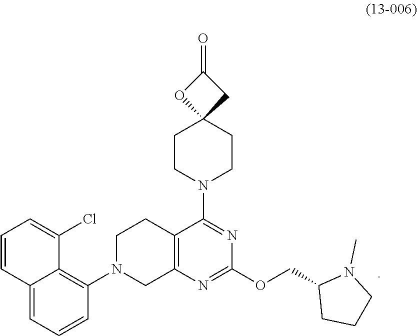

In embodiments, the compound has the formula:

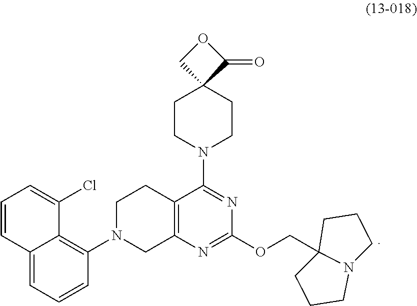

In embodiments, the compound has the formula:

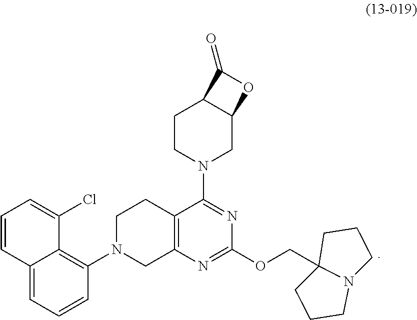 [SMILES: O=C1O[C@H]2CN(c3nc(OCC45CCCN4CCC5)nc4c3CCN(c3cccc5cccc(Cl)c35)C4)CC[C@@H]12]

In embodiments, the compound has the formula:

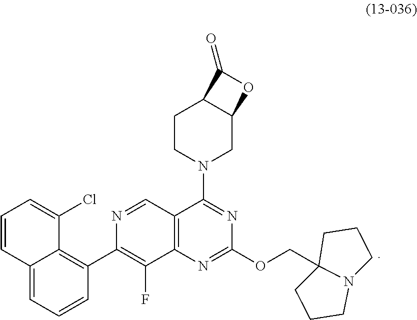 [SMILES: O=C1O[C@H]2CN(c3nc(OCC45CCCN4CCC5)nc4c(F)c(-c5cccc6cccc(Cl)c56)ncc34)CC[C@@H]12]

In embodiments, the compound has the formula:

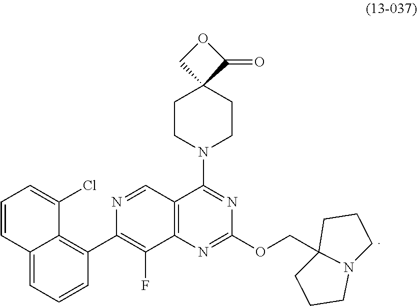

In embodiments, the compound has the formula:

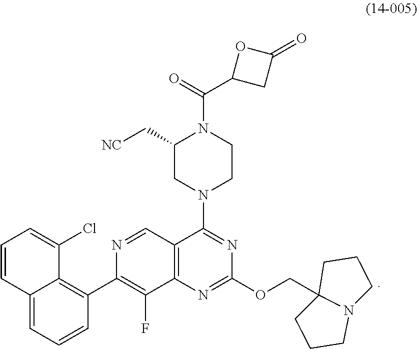 [SMILES: N#CC[C@H]1CN(c2nc(OCC34CCCN3CCC4)nc3c(F)c(-c4cccc5cccc(Cl)c45)ncc23)CCN1C(=O)C1CC(=O)O1]

In embodiments, the compound has the formula:

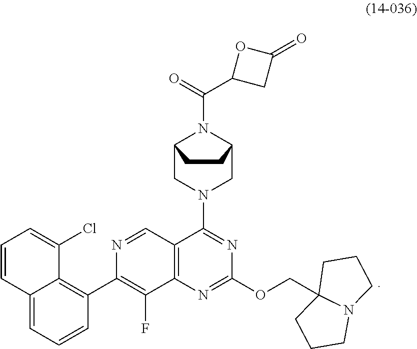

In embodiments, the compound has the formula:

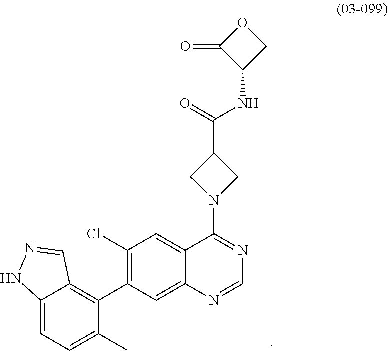 [SMILES: Cc1ccc2[nH]ncc2c1-c1cc2ncnc(N3CC(C(=O)N[C@H]4COC4=O)C3)c2cc1Cl]

In embodiments, the compound has the formula:

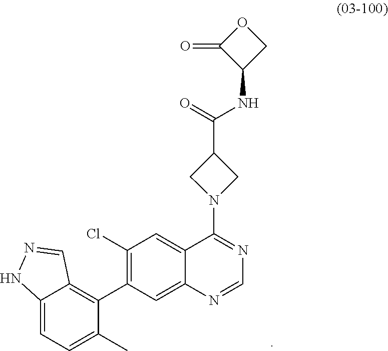 [SMILES: Cc1ccc2[nH]ncc2c1-c1cc2ncnc(N3CC(C(=O)N[C@@H]4COC4=O)C3)c2cc1Cl]

In embodiments, the compound has the formula:

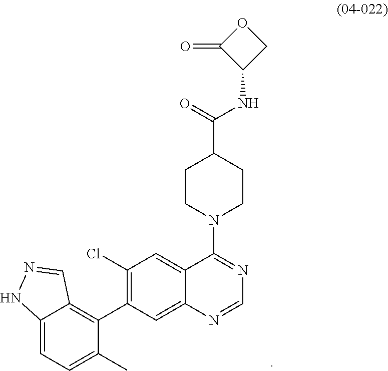 [SMILES: Cc1ccc2[nH]ncc2c1-c1cc2ncnc(N3CCC(C(=O)N[C@H]4COC4=O)CC3)c2cc1Cl]

In embodiments, the compound has the formula:

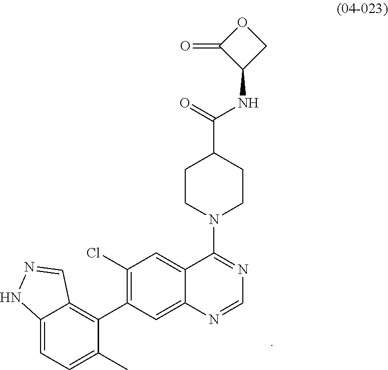 [SMILES: Cc1ccc2[nH]ncc2c1-c1cc2ncnc(N3CCC(C(=O)N[C@@H]4COC4=O)CC3)c2cc1Cl]

In embodiments, the compound has the formula:

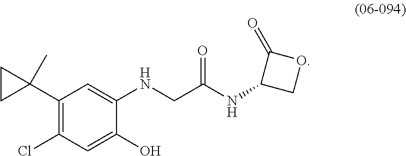 [SMILES: CC1(c2cc(NCC(=O)N[C@H]3COC3=O)c(O)cc2Cl)CC1]

In embodiments, the compound has the formula:

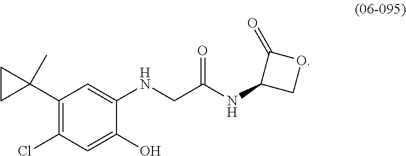 [SMILES: CC1(c2cc(NCC(=O)N[C@@H]3COC3=O)c(O)cc2Cl)CC1]

In embodiments, the compound has the formula:

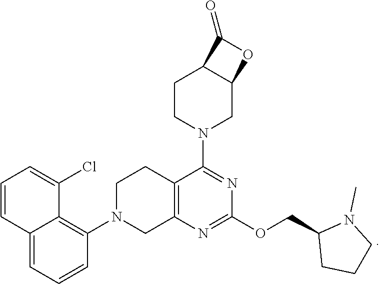 [SMILES: CN1CCC[C@H]1COc1nc2c(c(N3CC[C@H]4C(=O)O[C@H]4C3)n1)CCN(c1cccc3cccc(Cl)c13)C2]

In embodiments, the compound has the formula:

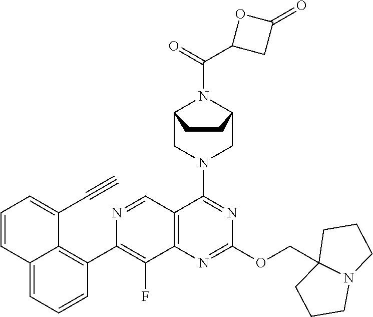

(14-049, also referred to herein as Compound 1 or (RS)-1). In embodiments, the compound has the formula:

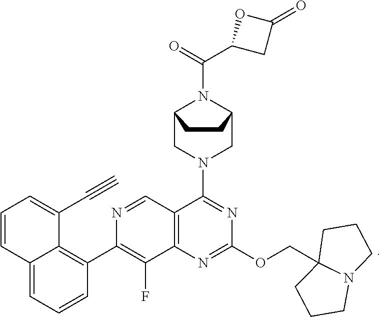

In embodiments, the compound has the formula:

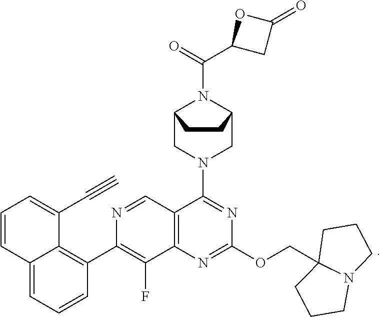

In embodiments, the compound has the formula:

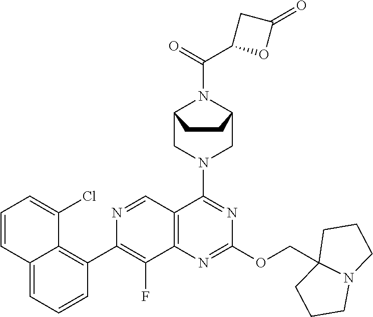

(14-075, also referred to herein as (S)-2). In embodiments, the compound has the formula:

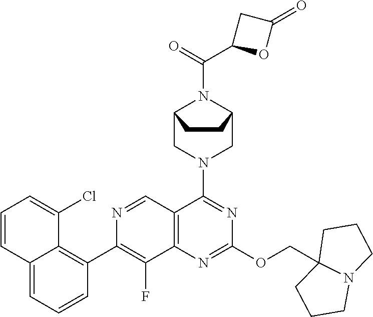

(14-076, also referred to herein as Compound 2 or (R)-2). In embodiments, the compound has the formula:

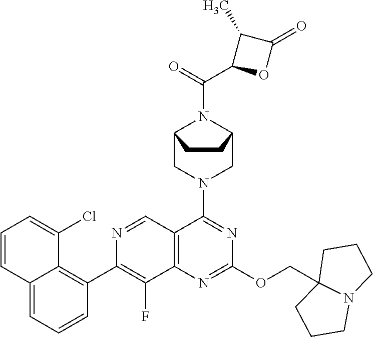

(14-082, also referred to herein as Compound 3 or (2R, 3S)-3). In embodiments, the compound has the formula:

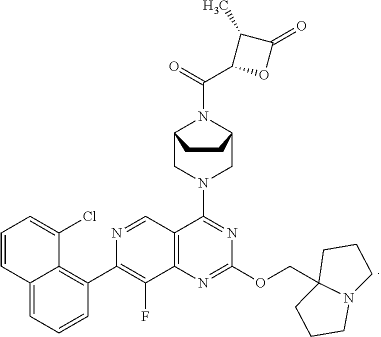

In embodiments, the compound has the formula:

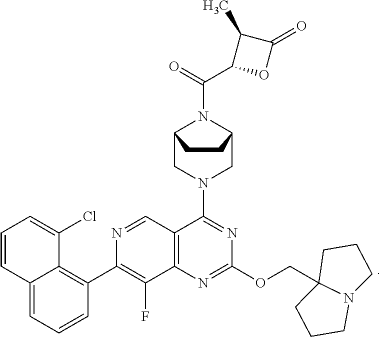

In embodiments, the compound has the formula:

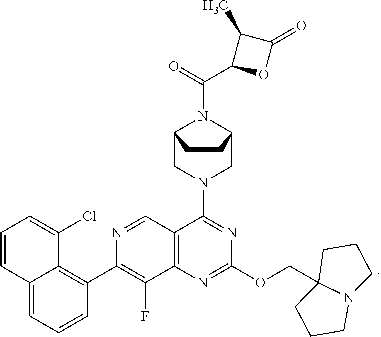

In embodiments, the compound has the formula:

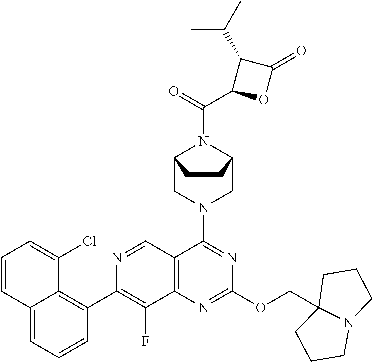

(15-011, also referred to herein as Compound 4 or (2R, 3S)-4). In embodiments, the compound has the formula:

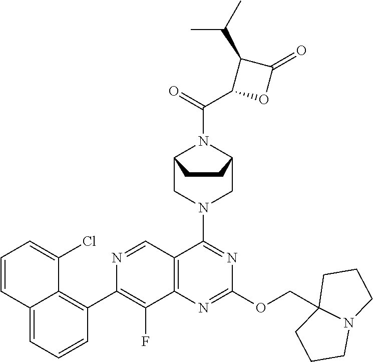

(15-014, also referred to herein as (2S, 3R)-4). In embodiments, the compound has the formula:

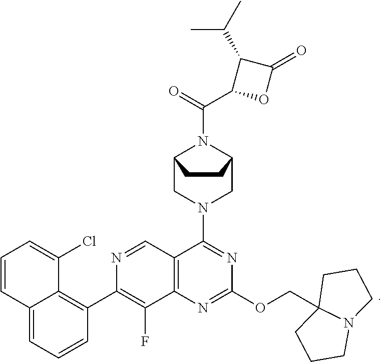

In embodiments, the compound has the formula:

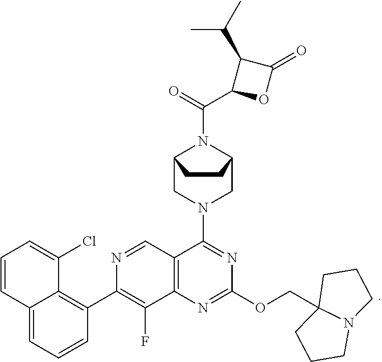

In embodiments, the compound has the formula:

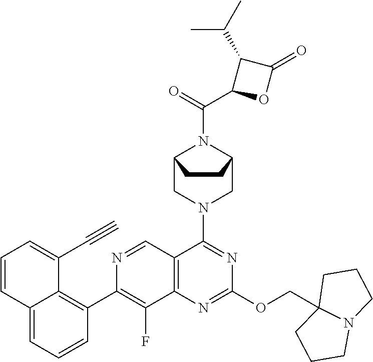

(Compound 5 or (2R, 3S)-5. In embodiments, the compound has the formula:

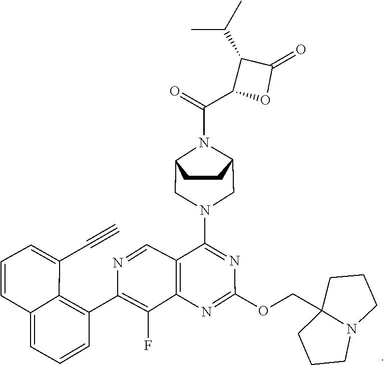

In embodiments, the compound has the formula:

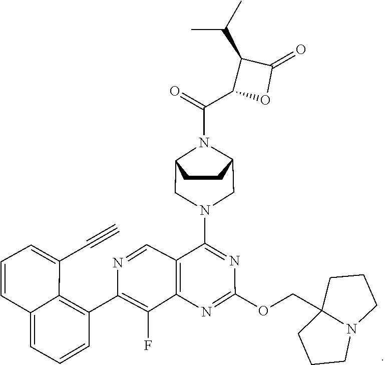

In embodiments, the compound has the formula:

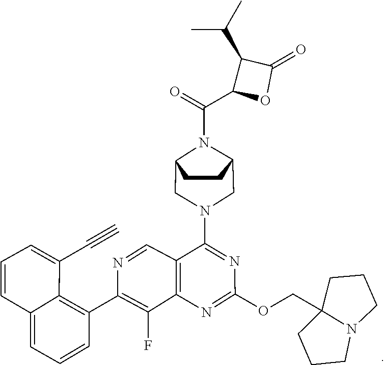

In embodiments, the compound has the formula:

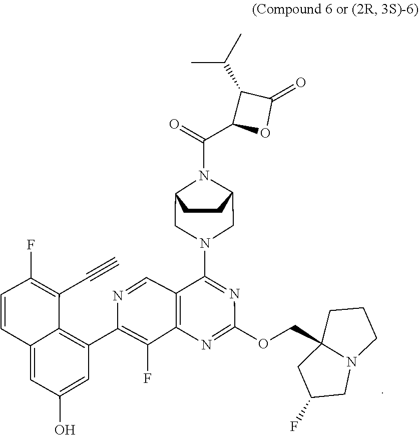

In embodiments, the compound has the formula:

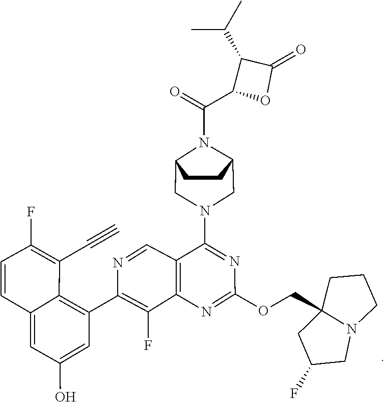

In embodiments, the compound has the formula:

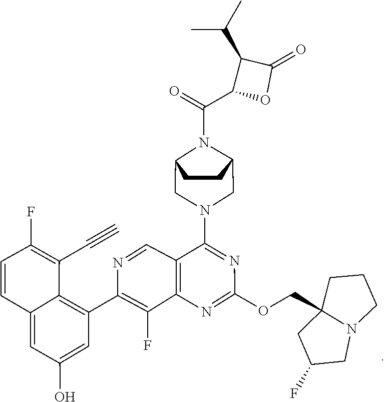

In embodiments, the compound has the formula:

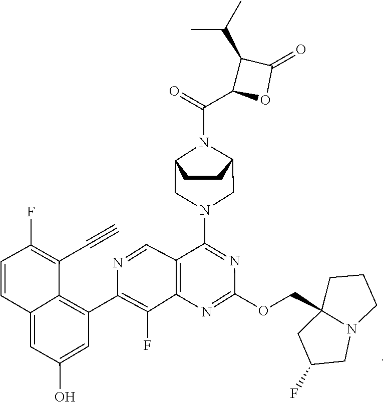

In embodiments, the compound has the formula:

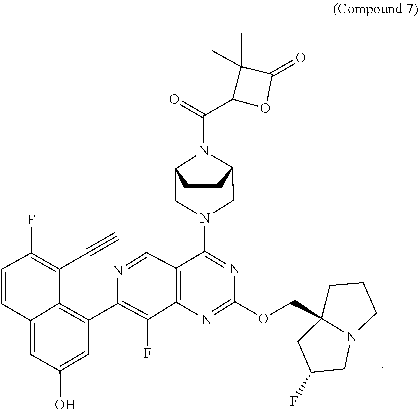

In embodiments, the compound has the formula:

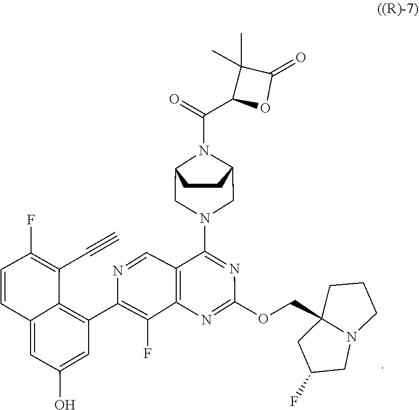

In embodiments, the compound has the formula:

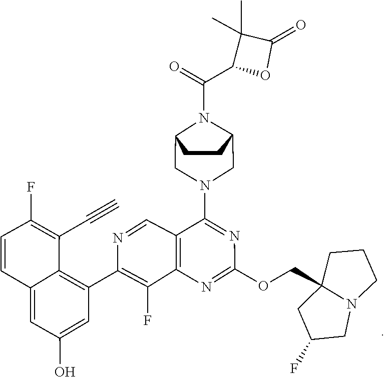

In embodiments, the compound binds Ras(G12S) (e.g., K-Ras(G12S), H-Ras(G12S), or N-Ras(G12S)) behind Switch II. In embodiments, the compound modulates the conformation of Switch II. In embodiments, the compound inhibits (e.g., by about 0.1, 0.2, 0.3, 0.4, 0.5, 0.6, 0.7, 0.8, 0.9, 1.0, 2.0, 3.0, 4.0, 5.0, 6.0, 7.0, 8.0, 9.0, 10, 11, 12, 13, 14, 15, 16, 17, 18, 19, 20, 25, 30, 35, 40, 45, 50, 55, 60, 65, 70, 75, 80, 85, 90, 95, 100, 200, 300, 400, 500, 600, 700, 800, 900, 1000, 2000, 3000, 4000, 5000, 6000, 7000, 8000, 9000, 10000 fold or more) Ras(G12S) (e.g., K-Ras(G12S), H-Ras(G12S), or N-Ras(G12S)) nucleotide exchange (e.g., GDP for GTP or GTP for GDP) relative to the absence of the compound. In embodiments, the compound inhibits release of GDP from Ras(G12S) (e.g., K-Ras(G12S), H-Ras(G12S), or N-Ras(G12S)) relative to the absence of the compound. In embodiments, the compound inhibits binding of GDP to Ras(G12S) (e.g., K-Ras(G12S), H-Ras(G12S), or N-Ras(G12S)) relative to the absence of the compound. In embodiments, the compound inhibits binding of GTP to Ras(G12S) (e.g., K-Ras(G12S), H-Ras(G12S), or N-Ras(G12S)) relative to the absence of the compound. In embodiments, the compound increases (e.g., by about 0.1, 0.2, 0.3, 0.4, 0.5, 0.6, 0.7, 0.8, 0.9, 1.0, 2.0, 3.0, 4.0, 5.0, 6.0, 7.0, 8.0, 9.0, 10, 11, 12, 13, 14, 15, 16, 17, 18, 19, 20, 25, 30, 35, 40, 45, 50, 55, 60, 65, 70, 75, 80, 85, 90, 95, 100, 200, 300, 400, 500, 600, 700, 800, 900, 1000, 2000, 3000, 4000, 5000, 6000, 7000, 8000, 9000, 10000 fold or more) Ras(G12S) (e.g., K-Ras(G12S), H-Ras(G12S), or N-Ras(G12S)) nucleotide exchange (e.g., GDP for GTP or GTP for GDP) relative to the absence of the compound. In embodiments, the compound increases release of GDP from Ras(G12S) (e.g., K-Ras(G12S), H-Ras(G12S), or N-Ras(G12S)) relative to the absence of the compound. In embodiments, the compound increases release of GTP from Ras(G12S) (e.g., K-Ras(G12S), H-Ras(G12S), or N-Ras(G12S)) relative to the absence of the compound. In embodiments, the compound increases binding of GDP to Ras(G12S) (e.g., K-Ras(G12S), H-Ras(G12S), or N-Ras(G12S)) relative to the absence of the compound. In embodiments, the compound inhibits binding of GTP to Ras(G12S) (e.g., K-Ras(G12S), H-Ras(G12S), or N-Ras(G12S)) relative to the absence of the compound. In embodiments, the compound inhibits binding of a GTP analog (e.g., mant-dGTP) to Ras(G12S) (e.g., K-Ras(G12S), H-Ras(G12S), or N-Ras(G12S)) relative to the absence of the compound. In embodiments, the compound modulates the conformation of a Ras(G12S) (e.g., K-Ras(G12S), H-Ras(G12S), or N-Ras(G12S)) amino acid that contacts GTP in the absence of the compound. In embodiments, the compound modulates the conformation of a Ras(G12S) (e.g., K-Ras(G12S), H-Ras(G12S), or N-Ras(G12S)) amino acid that contacts GDP in the absence of the compound. In embodiments, the compound modulates the conformation of a plurality of Ras(G12S) (e.g., K-Ras(G12S), H-Ras(G12S), or N-Ras(G12S)) amino acids that contact GTP in the absence of the compound. In embodiments, the compound modulates the conformation of a plurality of Ras(G12S) (e.g., K-Ras(G12S), H-Ras(G12S), or N-Ras(G12S)) amino acids that contact GDP in the absence of the compound. In embodiments, the compound modulates the binding of GTP and/or GDP to Ras(G12S) (e.g., K-Ras(G12S), H-Ras(G12S), or N-Ras(G12S)) compared to binding in the absence of the compound. In embodiments, the compound modulates the release of GTP and/or GDP from Ras(G12S) (e.g., K-Ras(G12S), H-Ras(G12S), or N-Ras(G12S)) compared to release in the absence of the compound. In embodiments, the compound modulates the ratio of the binding of GTP and GDP to Ras(G12S) (e.g., K-Ras(G12S), H-Ras(G12S), or N-Ras(G12S)) compared to the ratio in the absence of the compound. In embodiments, the compound modulates the ratio of the rate of release of GTP and GDP from Ras(G12S) (e.g., K-Ras(G12S), H-Ras(G12S), or N-Ras(G12S)) compared to the ratio in the absence of the compound. In embodiments, the compound binds Ras(G12S) (e.g., K-Ras(G12S), H-Ras(G12S), or N-Ras(G12S)) protein bound to GDP and, after release of the GDP, modulates the subsequent binding of GDP or GTP to the Ras bound to the compound. In embodiments, the compound binds Ras(G12S) (e.g., K-Ras(G12S), H-Ras(G12S), or N-Ras(G12S)) protein bound to GDP and, after release of the GDP, modulates the subsequent binding of GDP to the Ras bound to the compound. In embodiments, the compound binds Ras(G12S) (e.g., K-Ras(G12S), H-Ras(G12S), or N-Ras(G12S)) protein bound to GDP and after release of the GDP, modulates the subsequent binding of GTP to the Ras bound to the compound.

In embodiments, the compound binds Ras(G13S) (e.g., K-Ras(G13S), H-Ras(G13S), or N-Ras(G13S)) behind Switch II. In embodiments, the compound modulates the conformation of Switch II. In embodiments, the compound inhibits (e.g., by about 0.1, 0.2, 0.3, 0.4, 0.5, 0.6, 0.7, 0.8, 0.9, 1.0, 2.0, 3.0, 4.0, 5.0, 6.0, 7.0, 8.0, 9.0, 10, 11, 12, 13, 14, 15, 16, 17, 18, 19, 20, 25, 30, 35, 40, 45, 50, 55, 60, 65, 70, 75, 80, 85, 90, 95, 100, 200, 300, 400, 500, 600, 700, 800, 900, 1000, 2000, 3000, 4000, 5000, 6000, 7000, 8000, 9000, 10000 fold or more) Ras(G13S) (e.g., K-Ras(G13S), H-Ras(G13S), or N-Ras(G13S)) nucleotide exchange (e.g., GDP for GTP or GTP for GDP) relative to the absence of the compound. In embodiments, the compound inhibits release of GDP from Ras(G13S) (e.g., K-Ras(G13S), H-Ras(G13S), or N-Ras(G13S)) relative to the absence of the compound. In embodiments, the compound inhibits binding of GDP to Ras(G13S) (e.g., K-Ras(G13S), H-Ras(G13S), or N-Ras(G13S)) relative to the absence of the compound. In embodiments, the compound inhibits binding of GTP to Ras(G13S) (e.g., K-Ras(G13S), H-Ras(G13S), or N-Ras(G13S)) relative to the absence of the compound. In embodiments, the compound increases (e.g., by about 0.1, 0.2, 0.3, 0.4, 0.5, 0.6, 0.7, 0.8, 0.9, 1.0, 2.0, 3.0, 4.0, 5.0, 6.0, 7.0, 8.0, 9.0, 10, 11, 12, 13, 14, 15, 16, 17, 18, 19, 20, 25, 30, 35, 40, 45, 50, 55, 60, 65, 70, 75, 80, 85, 90, 95, 100, 200, 300, 400, 500, 600, 700, 800, 900, 1000, 2000, 3000, 4000, 5000, 6000, 7000, 8000, 9000, 10000 fold or more) Ras(G13S) (e.g., K-Ras(G13S), H-Ras(G13S), or N-Ras(G13S)) nucleotide exchange (e.g., GDP for GTP or GTP for GDP) relative to the absence of the compound. In embodiments, the compound increases release of GDP from Ras(G13S) (e.g., K-Ras(G13S), H-Ras(G13S), or N-Ras(G13S)) relative to the absence of the compound. In embodiments, the compound increases release of GTP from Ras(G13S) (e.g., K-Ras(G13S), H-Ras(G13S), or N-Ras(G13S)) relative to the absence of the compound. In embodiments, the compound increases binding of GDP to Ras(G13S) (e.g., K-Ras(G13S), H-Ras(G13S), or N-Ras(G13S)) relative to the absence of the compound. In embodiments, the compound inhibits binding of GTP to Ras(G13S) (e.g., K-Ras(G13S), H-Ras(G13S), or N-Ras(G13S)) relative to the absence of the compound. In embodiments, the compound inhibits binding of a GTP analog (e.g., mant-dGTP) to Ras(G13S) (e.g., K-Ras(G13S), H-Ras(G13S), or N-Ras(G13S)) relative to the absence of the compound. In embodiments, the compound modulates the conformation of a Ras(G13S) (e.g., K-Ras(G13S), H-Ras(G13S), or N-Ras(G13S)) amino acid that contacts GTP in the absence of the compound. In embodiments, the compound modulates the conformation of a Ras(G13S) (e.g., K-Ras(G13S), H-Ras(G13S), or N-Ras(G13S)) amino acid that contacts GDP in the absence of the compound. In embodiments, the compound modulates the conformation of a plurality of Ras(G13S) (e.g., K-Ras(G13S), H-Ras(G13S), or N-Ras(G13S)) amino acids that contact GTP in the absence of the compound. In embodiments, the compound modulates the conformation of a plurality of Ras(G13S) (e.g., K-Ras(G13S), H-Ras(G13S), or N-Ras(G13S)) amino acids that contact GDP in the absence of the compound. In embodiments, the compound modulates the binding of GTP and/or GDP to Ras(G13S) (e.g., K-Ras(G13S), H-Ras(G13S), or N-Ras(G13S)) compared to binding in the absence of the compound. In embodiments, the compound modulates the release of GTP and/or GDP from Ras(G13S) (e.g., K-Ras(G13S), H-Ras(G13S), or N-Ras(G13S)) compared to release in the absence of the compound. In embodiments, the compound modulates the ratio of the binding of GTP and GDP to Ras(G13S) (e.g., K-Ras(G13S), H-Ras(G13S), or N-Ras(G13S)) compared to the ratio in the absence of the compound. In embodiments, the compound modulates the ratio of the rate of release of GTP and GDP from Ras(G13S) (e.g., K-Ras(G13S), H-Ras(G13S), or N-Ras(G13S)) compared to the ratio in the absence of the compound. In embodiments, the compound binds Ras(G13S) (e.g., K-Ras(G13S), H-Ras(G13S), or N-Ras(G13S)) protein bound to GDP and, after release of the GDP, modulates the subsequent binding of GDP or GTP to the Ras bound to the compound. In embodiments, the compound binds Ras(G13S) (e.g., K-Ras(G13S), H-Ras(G13S), or N-Ras(G13S)) protein bound to GDP and, after release of the GDP, modulates the subsequent binding of GDP to the Ras bound to the compound. In embodiments, the compound binds Ras(G13S) (e.g., K-Ras(G13S), H-Ras(G13S), or N-Ras(G13S)) protein bound to GDP and after release of the GDP, modulates the subsequent binding of GTP to the Ras bound to the compound.

In embodiments, the compound binds Ras(G12T) (e.g., K-Ras(G12T), H-Ras(G12T), or N-Ras(G12T)) behind Switch II. In embodiments, the compound modulates the conformation of Switch II. In embodiments, the compound inhibits (e.g., by about 0.1, 0.2, 0.3, 0.4, 0.5, 0.6, 0.7, 0.8, 0.9, 1.0, 2.0, 3.0, 4.0, 5.0, 6.0, 7.0, 8.0, 9.0, 10, 11, 12, 13, 14, 15, 16, 17, 18, 19, 20, 25, 30, 35, 40, 45, 50, 55, 60, 65, 70, 75, 80, 85, 90, 95, 100, 200, 300, 400, 500, 600, 700, 800, 900, 1000, 2000, 3000, 4000, 5000, 6000, 7000, 8000, 9000, 10000 fold or more) Ras(G12T) (e.g., K-Ras(G12T), H-Ras(G12T), or N-Ras(G12T)) nucleotide exchange (e.g., GDP for GTP or GTP for GDP) relative to the absence of the compound. In embodiments, the compound inhibits release of GDP from Ras(G12T) (e.g., K-Ras(G12T), H-Ras(G12T), or N-Ras(G12T)) relative to the absence of the compound. In embodiments, the compound inhibits binding of GDP to Ras(G12T) (e.g., K-Ras(G12T), H-Ras(G12T), or N-Ras(G12T)) relative to the absence of the compound. In embodiments, the compound inhibits binding of GTP to Ras(G12T) (e.g., K-Ras(G12T), H-Ras(G12T), or N-Ras(G12T)) relative to the absence of the compound. In embodiments, the compound increases (e.g., by about 0.1, 0.2, 0.3, 0.4, 0.5, 0.6, 0.7, 0.8, 0.9, 1.0, 2.0, 3.0, 4.0, 5.0, 6.0, 7.0, 8.0, 9.0, 10, 11, 12, 13, 14, 15, 16, 17, 18, 19, 20, 25, 30, 35, 40, 45, 50, 55, 60, 65, 70, 75, 80, 85, 90, 95, 100, 200, 300, 400, 500, 600, 700, 800, 900, 1000, 2000, 3000, 4000, 5000, 6000, 7000, 8000, 9000, 10000 fold or more) Ras(G12T) (e.g., K-Ras(G12T), H-Ras(G12T), or N-Ras(G12T)) nucleotide exchange (e.g., GDP for GTP or GTP for GDP) relative to the absence of the compound. In embodiments, the compound increases release of GDP from Ras(G12T) (e.g., K-Ras(G12T), H-Ras(G12T), or N-Ras(G12T)) relative to the absence of the compound. In embodiments, the compound increases release of GTP from Ras(G12T) (e.g., K-Ras(G12T), H-Ras(G12T), or N-Ras(G12T)) relative to the absence of the compound. In embodiments, the compound increases binding of GDP to Ras(G12T) (e.g., K-Ras(G12T), H-Ras(G12T), or N-Ras(G12T)) relative to the absence of the compound. In embodiments, the compound inhibits binding of GTP to Ras(G12T) (e.g., K-Ras(G12T), H-Ras(G12T), or N-Ras(G12T)) relative to the absence of the compound. In embodiments, the compound inhibits binding of a GTP analog (e.g., mant-dGTP) to Ras(G12T) (e.g., K-Ras(G12T), H-Ras(G12T), or N-Ras(G12T)) relative to the absence of the compound. In embodiments, the compound modulates the conformation of a Ras(G12T) (e.g., K-Ras(G12T), H-Ras(G12T), or N-Ras(G12T)) amino acid that contacts GTP in the absence of the compound. In embodiments, the compound modulates the conformation of a Ras(G12T) (e.g., K-Ras(G12T), H-Ras(G12T), or N-Ras(G12T)) amino acid that contacts GDP in the absence of the compound. In embodiments, the compound modulates the conformation of a plurality of Ras(G12T) (e.g., K-Ras(G12T), H-Ras(G12T), or N-Ras(G12T)) amino acids that contact GTP in the absence of the compound. In embodiments, the compound modulates the conformation of a plurality of Ras(G12T) (e.g., K-Ras(G12T), H-Ras(G12T), or N-Ras(G12T)) amino acids that contact GDP in the absence of the compound. In embodiments, the compound modulates the binding of GTP and/or GDP to Ras(G12T) (e.g., K-Ras(G12T), H-Ras(G12T), or N-Ras(G12T)) compared to binding in the absence of the compound. In embodiments, the compound modulates the release of GTP and/or GDP from Ras(G12T) (e.g., K-Ras(G12T), H-Ras(G12T), or N-Ras(G12T)) compared to release in the absence of the compound. In embodiments, the compound modulates the ratio of the binding of GTP and GDP to Ras(G12T) (e.g., K-Ras(G12T), H-Ras(G12T), or N-Ras(G12T)) compared to the ratio in the absence of the compound. In embodiments, the compound modulates the ratio of the rate of release of GTP and GDP from Ras(G12T) (e.g., K-Ras(G12T), H-Ras(G12T), or N-Ras(G12T)) compared to the ratio in the absence of the compound. In embodiments, the compound binds Ras(G12T) (e.g., K-Ras(G12T), H-Ras(G12T), or N-Ras(G12T)) protein bound to GDP and, after release of the GDP, modulates the subsequent binding of GDP or GTP to the Ras bound to the compound. In embodiments, the compound binds Ras(G12T) (e.g., K-Ras(G12T), H-Ras(G12T), or N-Ras(G12T)) protein bound to GDP and, after release of the GDP, modulates the subsequent binding of GDP to the Ras bound to the compound. In embodiments, the compound binds Ras(G12T) (e.g., K-Ras(G12T), H-Ras(G12T), or N-Ras(G12T)) protein bound to GDP and after release of the GDP, modulates the subsequent binding of GTP to the Ras bound to the compound.

In embodiments, the compound binds Ras(G12D) (e.g., K-Ras(G12D), H-Ras(G12D), or N-Ras(G12D)) behind Switch II. In embodiments, the compound modulates the conformation of Switch II. In embodiments, the compound inhibits (e.g., by about 0.1, 0.2, 0.3, 0.4, 0.5, 0.6, 0.7, 0.8, 0.9, 1.0, 2.0, 3.0, 4.0, 5.0, 6.0, 7.0, 8.0, 9.0, 10, 11, 12, 13, 14, 15, 16, 17, 18, 19, 20, 25, 30, 35, 40, 45, 50, 55, 60, 65, 70, 75, 80, 85, 90, 95, 100, 200, 300, 400, 500, 600, 700, 800, 900, 1000, 2000, 3000, 4000, 5000, 6000, 7000, 8000, 9000, 10000 fold or more) Ras(G12D) (e.g., K-Ras(G12D), H-Ras(G12D), or N-Ras(G12D)) nucleotide exchange (e.g., GDP for GTP or GTP for GDP) relative to the absence of the compound. In embodiments, the compound inhibits release of GDP from Ras(G12D) (e.g., K-Ras(G12D), H-Ras(G12D), or N-Ras(G12D)) relative to the absence of the compound. In embodiments, the compound inhibits binding of GDP to Ras(G12D) (e.g., K-Ras(G12D), H-Ras(G12D), or N-Ras(G12D)) relative to the absence of the compound. In embodiments, the compound inhibits binding of GTP to Ras(G12D) (e.g., K-Ras(G12D), H-Ras(G12D), or N-Ras(G12D)) relative to the absence of the compound. In embodiments, the compound increases (e.g., by about 0.1, 0.2, 0.3, 0.4, 0.5, 0.6, 0.7, 0.8, 0.9, 1.0, 2.0, 3.0, 4.0, 5.0, 6.0, 7.0, 8.0, 9.0, 10, 11, 12, 13, 14, 15, 16, 17, 18, 19, 20, 25, 30, 35, 40, 45, 50, 55, 60, 65, 70, 75, 80, 85, 90, 95, 100, 200, 300, 400, 500, 600, 700, 800, 900, 1000, 2000, 3000, 4000, 5000, 6000, 7000, 8000, 9000, 10000 fold or more) Ras(G12D) (e.g., K-Ras(G12D), H-Ras(G12D), or N-Ras(G12D)) nucleotide exchange (e.g., GDP for GTP or GTP for GDP) relative to the absence of the compound. In embodiments, the compound increases release of GDP from Ras(G12D) (e.g., K-Ras(G12D), H-Ras(G12D), or N-Ras(G12D)) relative to the absence of the compound. In embodiments, the compound increases release of GTP from Ras(G12D) (e.g., K-Ras(G12D), H-Ras(G12D), or N-Ras(G12D)) relative to the absence of the compound. In embodiments, the compound increases binding of GDP to Ras(G12D) (e.g., K-Ras(G12D), H-Ras(G12D), or N-Ras(G12D)) relative to the absence of the compound. In embodiments, the compound inhibits binding of GTP to Ras(G12D) (e.g., K-Ras(G12D), H-Ras(G12D), or N-Ras(G12D)) relative to the absence of the compound. In embodiments, the compound inhibits binding of a GTP analog (e.g., mant-dGTP) to Ras(G12D) (e.g., K-Ras(G12D), H-Ras(G12D), or N-Ras(G12D)) relative to the absence of the compound. In embodiments, the compound modulates the conformation of a Ras(G12D) (e.g., K-Ras(G12D), H-Ras(G12D), or N-Ras(G12D)) amino acid that contacts GTP in the absence of the compound. In embodiments, the compound modulates the conformation of a Ras(G12D) (e.g., K-Ras(G12D), H-Ras(G12D), or N-Ras(G12D)) amino acid that contacts GDP in the absence of the compound. In embodiments, the compound modulates the conformation of a plurality of Ras(G12D) (e.g., K-Ras(G12D), H-Ras(G12D), or N-Ras(G12D)) amino acids that contact GTP in the absence of the compound. In embodiments, the compound modulates the conformation of a plurality of Ras(G12D) (e.g., K-Ras(G12D), H-Ras(G12D), or N-Ras(G12D)) amino acids that contact GDP in the absence of the compound. In embodiments, the compound modulates the binding of GTP and/or GDP to Ras(G12D) (e.g., K-Ras(G12D), H-Ras(G12D), or N-Ras(G12D)) compared to binding in the absence of the compound. In embodiments, the compound modulates the release of GTP and/or GDP from Ras(G12D) (e.g., K-Ras(G12D), H-Ras(G12D), or N-Ras(G12D)) compared to release in the absence of the compound. In embodiments, the compound modulates the ratio of the binding of GTP and GDP to Ras(G12D) (e.g., K-Ras(G12D), H-Ras(G12D), or N-Ras(G12D)) compared to the ratio in the absence of the compound. In embodiments, the compound modulates the ratio of the rate of release of GTP and GDP from Ras(G12D) (e.g., K-Ras(G12D), H-Ras(G12D), or N-Ras(G12D)) compared to the ratio in the absence of the compound. In embodiments, the compound binds Ras(G12D) (e.g., K-Ras(G12D), H-Ras(G12D), or N-Ras(G12D)) protein bound to GDP and, after release of the GDP, modulates the subsequent binding of GDP or GTP to the Ras bound to the compound. In embodiments, the compound binds Ras(G12D) (e.g., K-Ras(G12D), H-Ras(G12D), or N-Ras(G12D)) protein bound to GDP and, after release of the GDP, modulates the subsequent binding of GDP to the Ras bound to the compound. In embodiments, the compound binds Ras(G12D) (e.g., K-Ras(G12D), H-Ras(G12D), or N-Ras(G12D)) protein bound to GDP and after release of the GDP, modulates the subsequent binding of GTP to the Ras bound to the compound.

In embodiments, the compound binds Ras(G13D) (e.g., K-Ras(G13D), H-Ras(G13D), or N-Ras(G13D)) behind Switch II. In embodiments, the compound modulates the conformation of Switch II. In embodiments, the compound inhibits (e.g., by about 0.1, 0.2, 0.3, 0.4, 0.5, 0.6, 0.7, 0.8, 0.9, 1.0, 2.0, 3.0, 4.0, 5.0, 6.0, 7.0, 8.0, 9.0, 10, 11, 12, 13, 14, 15, 16, 17, 18, 19, 20, 25, 30, 35, 40, 45, 50, 55, 60, 65, 70, 75, 80, 85, 90, 95, 100, 200, 300, 400, 500, 600, 700, 800, 900, 1000, 2000, 3000, 4000, 5000, 6000, 7000, 8000, 9000, 10000 fold or more) Ras(G13D) (e.g., K-Ras(G13D), H-Ras(G13D), or N-Ras(G13D)) nucleotide exchange (e.g., GDP for GTP or GTP for GDP) relative to the absence of the compound. In embodiments, the compound inhibits release of GDP from Ras(G13D) (e.g., K-Ras(G13D), H-Ras(G13D), or N-Ras(G13D)) relative to the absence of the compound. In embodiments, the compound inhibits binding of GDP to Ras(G13D) (e.g., K-Ras(G13D), H-Ras(G13D), or N-Ras(G13D)) relative to the absence of the compound. In embodiments, the compound inhibits binding of GTP to Ras(G13D) (e.g., K-Ras(G13D), H-Ras(G13D), or N-Ras(G13D)) relative to the absence of the compound. In embodiments, the compound increases (e.g., by about 0.1, 0.2, 0.3, 0.4, 0.5, 0.6, 0.7, 0.8, 0.9, 1.0, 2.0, 3.0, 4.0, 5.0, 6.0, 7.0, 8.0, 9.0, 10, 11, 12, 13, 14, 15, 16, 17, 18, 19, 20, 25, 30, 35, 40, 45, 50, 55, 60, 65, 70, 75, 80, 85, 90, 95, 100, 200, 300, 400, 500, 600, 700, 800, 900, 1000, 2000, 3000, 4000, 5000, 6000, 7000, 8000, 9000, 10000 fold or more) Ras(G13D) (e.g., K-Ras(G13D), H-Ras(G13D), or N-Ras(G13D)) nucleotide exchange (e.g., GDP for GTP or GTP for GDP) relative to the absence of the compound. In embodiments, the compound increases release of GDP from Ras(G13D) (e.g., K-Ras(G13D), H-Ras(G13D), or N-Ras(G13D)) relative to the absence of the compound. In embodiments, the compound increases release of GTP from Ras(G13D) (e.g., K-Ras(G13D), H-Ras(G13D), or N-Ras(G13D)) relative to the absence of the compound. In embodiments, the compound increases binding of GDP to Ras(G13D) (e.g., K-Ras(G13D), H-Ras(G13D), or N-Ras(G13D)) relative to the absence of the compound. In embodiments, the compound inhibits binding of GTP to Ras(G13D) (e.g., K-Ras(G13D), H-Ras(G13D), or N-Ras(G13D)) relative to the absence of the compound. In embodiments, the compound inhibits binding of a GTP analog (e.g., mant-dGTP) to Ras(G13D) (e.g., K-Ras(G13D), H-Ras(G13D), or N-Ras(G13D)) relative to the absence of the compound. In embodiments, the compound modulates the conformation of a Ras(G13D) (e.g., K-Ras(G13D), H-Ras(G13D), or N-Ras(G13D)) amino acid that contacts GTP in the absence of the compound. In embodiments, the compound modulates the conformation of a Ras(G13D) (e.g., K-Ras(G13D), H-Ras(G13D), or N-Ras(G13D)) amino acid that contacts GDP in the absence of the compound. In embodiments, the compound modulates the conformation of a plurality of Ras(G13D) (e.g., K-Ras(G13D), H-Ras(G13D), or N-Ras(G13D)) amino acids that contact GTP in the absence of the compound. In embodiments, the compound modulates the conformation of a plurality of Ras(G13D) (e.g., K-Ras(G13D), H-Ras(G13D), or N-Ras(G13D)) amino acids that contact GDP in the absence of the compound. In embodiments, the compound modulates the binding of GTP and/or GDP to Ras(G13D) (e.g., K-Ras(G13D), H-Ras(G13D), or N-Ras(G13D)) compared to binding in the absence of the compound. In embodiments, the compound modulates the release of GTP and/or GDP from Ras(G13D) (e.g., K-Ras(G13D), H-Ras(G13D), or N-Ras(G13D)) compared to release in the absence of the compound. In embodiments, the compound modulates the ratio of the binding of GTP and GDP to Ras(G13D) (e.g., K-Ras(G13D), H-Ras(G13D), or N-Ras(G13D)) compared to the ratio in the absence of the compound. In embodiments, the compound modulates the ratio of the rate of release of GTP and GDP from Ras(G13D) (e.g., K-Ras(G13D), H-Ras(G13D), or N-Ras(G13D)) compared to the ratio in the absence of the compound. In embodiments, the compound binds Ras(G13D) (e.g., K-Ras(G13D), H-Ras(G13D), or N-Ras(G13D)) protein bound to GDP and, after release of the GDP, modulates the subsequent binding of GDP or GTP to the Ras bound to the compound. In embodiments, the compound binds Ras(G13D) (e.g., K-Ras(G13D), H-Ras(G13D), or N-Ras(G13D)) protein bound to GDP and, after release of the GDP, modulates the subsequent binding of GDP to the Ras bound to the compound. In embodiments, the compound binds Ras(G13D) (e.g., K-Ras(G13D), H-Ras(G13D), or N-Ras(G13D)) protein bound to GDP and after release of the GDP, modulates the subsequent binding of GTP to the Ras bound to the compound.

In embodiments, the compound binds Ras(G12E) (e.g., K-Ras(G12E), H-Ras(G12E), or N-Ras(G12E)) behind Switch II. In embodiments, the compound modulates the conformation of Switch II. In embodiments, the compound inhibits (e.g., by about 0.1, 0.2, 0.3, 0.4, 0.5, 0.6, 0.7, 0.8, 0.9, 1.0, 2.0, 3.0, 4.0, 5.0, 6.0, 7.0, 8.0, 9.0, 10, 11, 12, 13, 14, 15, 16, 17, 18, 19, 20, 25, 30, 35, 40, 45, 50, 55, 60, 65, 70, 75, 80, 85, 90, 95, 100, 200, 300, 400, 500, 600, 700, 800, 900, 1000, 2000, 3000, 4000, 5000, 6000, 7000, 8000, 9000, 10000 fold or more) Ras(G12E) (e.g., K-Ras(G12E), H-Ras(G12E), or N-Ras(G12E)) nucleotide exchange (e.g., GDP for GTP or GTP for GDP) relative to the absence of the compound. In embodiments, the compound inhibits release of GDP from Ras(G12E) (e.g., K-Ras(G12E), H-Ras(G12E), or N-Ras(G12E)) relative to the absence of the compound. In embodiments, the compound inhibits binding of GDP to Ras(G12E) (e.g., K-Ras(G12E), H-Ras(G12E), or N-Ras(G12E)) relative to the absence of the compound. In embodiments, the compound inhibits binding of GTP to Ras(G12E) (e.g., K-Ras(G12E), H-Ras(G12E), or N-Ras(G12E)) relative to the absence of the compound. In embodiments, the compound increases (e.g., by about 0.1, 0.2, 0.3, 0.4, 0.5, 0.6, 0.7, 0.8, 0.9, 1.0, 2.0, 3.0, 4.0, 5.0, 6.0, 7.0, 8.0, 9.0, 10, 11, 12, 13, 14, 15, 16, 17, 18, 19, 20, 25, 30, 35, 40, 45, 50, 55, 60, 65, 70, 75, 80, 85, 90, 95, 100, 200, 300, 400, 500, 600, 700, 800, 900, 1000, 2000, 3000, 4000, 5000, 6000, 7000, 8000, 9000, 10000 fold or more) Ras(G12E) (e.g., K-Ras(G12E), H-Ras(G12E), or N-Ras(G12E)) nucleotide exchange (e.g., GDP for GTP or GTP for GDP) relative to the absence of the compound. In embodiments, the compound increases release of GDP from Ras(G12E) (e.g., K-Ras(G12E), H-Ras(G12E), or N-Ras(G12E)) relative to the absence of the compound. In embodiments, the compound increases release of GTP from Ras(G12E) (e.g., K-Ras(G12E), H-Ras(G12E), or N-Ras(G12E)) relative to the absence of the compound. In embodiments, the compound increases binding of GDP to Ras(G12E) (e.g., K-Ras(G12E), H-Ras(G12E), or N-Ras(G12E)) relative to the absence of the compound. In embodiments, the compound inhibits binding of GTP to Ras(G12E) (e.g., K-Ras(G12E), H-Ras(G12E), or N-Ras(G12E)) relative to the absence of the compound. In embodiments, the compound inhibits binding of a GTP analog (e.g., mant-dGTP) to Ras(G12E) (e.g., K-Ras(G12E), H-Ras(G12E), or N-Ras(G12E)) relative to the absence of the compound. In embodiments, the compound modulates the conformation of a Ras(G12E) (e.g., K-Ras(G12E), H-Ras(G12E), or N-Ras(G12E)) amino acid that contacts GTP in the absence of the compound. In embodiments, the compound modulates the conformation of a Ras(G12E) (e.g., K-Ras(G12E), H-Ras(G12E), or N-Ras(G12E)) amino acid that contacts GDP in the absence of the compound. In embodiments, the compound modulates the conformation of a plurality of Ras(G12E) (e.g., K-Ras(G12E), H-Ras(G12E), or N-Ras(G12E)) amino acids that contact GTP in the absence of the compound. In embodiments, the compound modulates the conformation of a plurality of Ras(G12E) (e.g., K-Ras(G12E), H-Ras(G12E), or N-Ras(G12E)) amino acids that contact GDP in the absence of the compound. In embodiments, the compound modulates the binding of GTP and/or GDP to Ras(G12E) (e.g., K-Ras(G12E), H-Ras(G12E), or N-Ras(G12E)) compared to binding in the absence of the compound. In embodiments, the compound modulates the release of GTP and/or GDP from Ras(G12E) (e.g., K-Ras(G12E), H-Ras(G12E), or N-Ras(G12E)) compared to release in the absence of the compound. In embodiments, the compound modulates the ratio of the binding of GTP and GDP to Ras(G12E) (e.g., K-Ras(G12E), H-Ras(G12E), or N-Ras(G12E)) compared to the ratio in the absence of the compound. In embodiments, the compound modulates the ratio of the rate of release of GTP and GDP from Ras(G12E) (e.g., K-Ras(G12E), H-Ras(G12E), or N-Ras(G12E)) compared to the ratio in the absence of the compound. In embodiments, the compound binds Ras(G12E) (e.g., K-Ras(G12E), H-Ras(G12E), or N-Ras(G12E)) protein bound to GDP and, after release of the GDP, modulates the subsequent binding of GDP or GTP to the Ras bound to the compound. In embodiments, the compound binds Ras(G12E) (e.g., K-Ras(G12E), H-Ras(G12E), or N-Ras(G12E)) protein bound to GDP and, after release of the GDP, modulates the subsequent binding of GDP to the Ras bound to the compound. In embodiments, the compound binds Ras(G12E) (e.g., K-Ras(G12E), H-Ras(G12E), or N-Ras(G12E)) protein bound to GDP and after release of the GDP, modulates the subsequent binding of GTP to the Ras bound to the compound.

In embodiments, the compound contacts the Switch II Binding Pocket of human K-Ras protein. In embodiments, the compound contacts a Switch II Binding Pocket amino acid corresponding to G60, Q61, D69, D92, H95, Y96, or Q99 of human K-Ras protein. In embodiments, the compound contacts a Switch II Binding Pocket amino acid corresponding to G60 of human K-Ras protein. In embodiments, the compound contacts a Switch II Binding Pocket amino acid corresponding to Q61 of human K-Ras protein. In embodiments, the compound contacts a Switch II Binding Pocket amino acid corresponding to D69 of human K-Ras protein. In embodiments, the compound contacts a Switch II Binding Pocket amino acid corresponding to D92 of human K-Ras protein. In embodiments, the compound contacts a Switch II Binding Pocket amino acid corresponding to H95 of human K-Ras protein. In embodiments, the compound contacts a Switch II Binding Pocket amino acid corresponding to Y96 of human K-Ras protein. In embodiments, the compound contacts a Switch II Binding Pocket amino acid corresponding to Q99 of human K-Ras protein.

In embodiments, the compound contacts the Switch II Binding Pocket of human H-Ras protein. In embodiments, the compound contacts a Switch II Binding Pocket amino acid corresponding to G60, Q61, D69, D92, Q95, Y96, or Q99 of human H-Ras protein. In embodiments, the compound contacts a Switch II Binding Pocket amino acid corresponding to G60 of human H-Ras protein. In embodiments, the compound contacts a Switch II Binding Pocket amino acid corresponding to Q61 of human H-Ras protein. In embodiments, the compound contacts a Switch II Binding Pocket amino acid corresponding to D69 of human H-Ras protein. In embodiments, the compound contacts a Switch II Binding Pocket amino acid corresponding to D92 of human H-Ras protein. In embodiments, the compound contacts a Switch II Binding Pocket amino acid corresponding to Q95 of human H-Ras protein. In embodiments, the compound contacts a Switch II Binding Pocket amino acid corresponding to Y96 of human H-Ras protein. In embodiments, the compound contacts a Switch II Binding Pocket amino acid corresponding to Q99 of human H-Ras protein.

In embodiments, the compound contacts the Switch II Binding Pocket of human N-Ras protein. In embodiments, the compound contacts a Switch II Binding Pocket amino acid corresponding to G60, Q61, D69, D92, L95, Y96, or Q99 of human N-Ras protein. In embodiments, the compound contacts a Switch II Binding Pocket amino acid corresponding to G60 of human N-Ras protein. In embodiments, the compound contacts a Switch II Binding Pocket amino acid corresponding to Q61 of human N-Ras protein. In embodiments, the compound contacts a Switch II Binding Pocket amino acid corresponding to D69 of human N-Ras protein. In embodiments, the compound contacts a Switch II Binding Pocket amino acid corresponding to D92 of human N-Ras protein. In embodiments, the compound contacts a Switch II Binding Pocket amino acid corresponding to L95 of human N-Ras protein. In embodiments, the compound contacts a Switch II Binding Pocket amino acid corresponding to Y96 of human N-Ras protein. In embodiments, the compound contacts a Switch II Binding Pocket amino acid corresponding to Q99 of human N-Ras protein.

In embodiments, the compound binds a human Ras(G12S) (e.g., human K-Ras(G12S), human H-Ras(G12S), or human N-Ras(G12S)) protein-GDP complex more strongly than the compound binds a human Ras(G12S) (e.g., human K-Ras(G12S), human H-Ras(G12S), or human N-Ras(G12S)) protein-GTP complex under identical conditions. In embodiments, the compound binds a human Ras(G12S) (e.g., human K-Ras(G12S), human H-Ras(G12S), or human N-Ras(G12S)) protein-GDP complex at least 2-fold stronger than the compound binds a human Ras(G12S) (e.g., human K-Ras(G12S), human H-Ras(G12S), or human N-Ras(G12S)) protein-GTP complex under identical conditions. In embodiments, the compound binds a human Ras(G12S) (e.g., human K-Ras(G12S), human H-Ras(G12S), or human N-Ras(G12S)) protein-GDP complex at least 5-fold stronger than the compound binds a human Ras(G12S) (e.g., human K-Ras(G12S), human H-Ras(G12S), or human N-Ras(G12S)) protein-GTP complex under identical conditions. In embodiments, the compound binds a human Ras(G12S) (e.g., human K-Ras(G12S), human H-Ras(G12S), or human N-Ras(G12S)) protein-GDP complex at least 10-fold stronger than the compound binds a human Ras(G12S) (e.g., human K-Ras(G12S), human H-Ras(G12S), or human N-Ras(G12S)) protein-GTP complex under identical conditions. In embodiments, the compound binds a human Ras(G12S) (e.g., human K-Ras(G12S), human H-Ras(G12S), or human N-Ras(G12S)) protein-GDP complex at least 20-fold stronger than the compound binds a human Ras(G12S) (e.g., human K-Ras(G12S), human H-Ras(G12S), or human N-Ras(G12S)) protein-GTP complex under identical conditions. In embodiments, the compound binds a human Ras(G12S) (e.g., human K-Ras(G12S), human H-Ras(G12S), or human N-Ras(G12S)) protein-GDP complex at least 40-fold stronger than said compound binds a human Ras(G12S) (e.g., human K-Ras(G12S), human H-Ras(G12S), or human N-Ras(G12S)) protein-GTP complex under identical conditions. In embodiments, the compound binds a human Ras(G12S) (e.g., human K-Ras(G12S), human H-Ras(G12S), or human N-Ras(G12S)) protein-GDP complex at least 60-fold stronger than the compound binds a human Ras(G12S) (e.g., human K-Ras(G12S), human H-Ras(G12S), or human N-Ras(G12S)) protein-GTP complex under identical conditions. In embodiments, the compound binds a human Ras(G12S) (e.g., human K-Ras(G12S), human H-Ras(G12S), or human N-Ras(G12S)) protein-GDP complex at least 80-fold stronger than the compound binds a human Ras(G12S) (e.g., human K-Ras(G12S), human H-Ras(G12S), or human N-Ras(G12S)) protein-GTP complex under identical conditions. In embodiments, the compound binds a human Ras(G12S) (e.g., human K-Ras(G12S), human H-Ras(G12S), or human N-Ras(G12S)) protein-GDP complex at least 100-fold stronger than said compound binds a human Ras(G12S) (e.g., human K-Ras(G12S), human H-Ras(G12S), or human N-Ras(G12S)) protein-GTP complex under identical conditions. In embodiments, the compound binds a human Ras(G12S) (e.g., human K-Ras(G12S), human H-Ras(G12S), or human N-Ras(G12S)) protein-GDP complex at least 500-fold stronger than the compound binds a human Ras(G12S) (e.g., human K-Ras(G12S), human H-Ras(G12S), or human N-Ras(G12S)) protein-GTP complex under identical conditions.

In embodiments, the compound binds a human Ras(G13S) (e.g., human K-Ras(G13S), human H-Ras(G13S), or human N-Ras(G13S)) protein-GDP complex more strongly than the compound binds a human Ras(G13S) (e.g., human K-Ras(G13S), human H-Ras(G13S), or human N-Ras(G13S)) protein-GTP complex under identical conditions. In embodiments, the compound binds a human Ras(G13S) (e.g., human K-Ras(G13S), human H-Ras(G13S), or human N-Ras(G13S)) protein-GDP complex at least 2-fold stronger than the compound binds a human Ras(G13S) (e.g., human K-Ras(G13S), human H-Ras(G13S), or human N-Ras(G13S)) protein-GTP complex under identical conditions. In embodiments, the compound binds a human Ras(G13S) (e.g., human K-Ras(G13S), human H-Ras(G13S), or human N-Ras(G13S)) protein-GDP complex at least 5-fold stronger than the compound binds a human Ras(G13S) (e.g., human K-Ras(G13S), human H-Ras(G13S), or human N-Ras(G13S)) protein-GTP complex under identical conditions. In embodiments, the compound binds a human Ras(G13S) (e.g., human K-Ras(G13S), human H-Ras(G13S), or human N-Ras(G13S)) protein-GDP complex at least 10-fold stronger than the compound binds a human Ras(G13S) (e.g., human K-Ras(G13S), human H-Ras(G13S), or human N-Ras(G13S)) protein-GTP complex under identical conditions. In embodiments, the compound binds a human Ras(G13S) (e.g., human K-Ras(G13S), human H-Ras(G13S), or human N-Ras(G13S)) protein-GDP complex at least 20-fold stronger than the compound binds a human Ras(G13S) (e.g., human K-Ras(G13S), human H-Ras(G13S), or human N-Ras(G13S)) protein-GTP complex under identical conditions. In embodiments, the compound binds a human Ras(G13S) (e.g., human K-Ras(G13S), human H-Ras(G13S), or human N-Ras(G13S)) protein-GDP complex at least 40-fold stronger than said compound binds a human Ras(G13S) (e.g., human K-Ras(G13S), human H-Ras(G13S), or human N-Ras(G13S)) protein-GTP complex under identical conditions. In embodiments, the compound binds a human Ras(G13S) (e.g., human K-Ras(G13S), human H-Ras(G13S), or human N-Ras(G13S)) protein-GDP complex at least 60-fold stronger than the compound binds a human Ras(G13S) (e.g., human K-Ras(G13S), human H-Ras(G13S), or human N-Ras(G13S)) protein-GTP complex under identical conditions. In embodiments, the compound binds a human Ras(G13S) (e.g., human K-Ras(G13S), human H-Ras(G13S), or human N-Ras(G13S)) protein-GDP complex at least 80-fold stronger than the compound binds a human Ras(G13S) (e.g., human K-Ras(G13S), human H-Ras(G13S), or human N-Ras(G13S)) protein-GTP complex under identical conditions. In embodiments, the compound binds a human Ras(G13S) (e.g., human K-Ras(G13S), human H-Ras(G13S), or human N-Ras(G13S)) protein-GDP complex at least 100-fold stronger than said compound binds a human Ras(G13S) (e.g., human K-Ras(G13S), human H-Ras(G13S), or human N-Ras(G13S)) protein-GTP complex under identical conditions. In embodiments, the compound binds a human Ras(G13S) (e.g., human K-Ras(G13S), human H-Ras(G13S), or human N-Ras(G13S)) protein-GDP complex at least 500-fold stronger than the compound binds a human Ras(G13S) (e.g., human K-Ras(G13S), human H-Ras(G13S), or human N-Ras(G13S)) protein-GTP complex under identical conditions.

In embodiments, the compound binds a human Ras(G12T) (e.g., human K-Ras(G12T), human H-Ras(G12T), or human N-Ras(G12T)) protein-GDP complex more strongly than the compound binds a human Ras(G12T) (e.g., human K-Ras(G12T), human H-Ras(G12T), or human N-Ras(G12T)) protein-GTP complex under identical conditions. In embodiments, the compound binds a human Ras(G12T) (e.g., human K-Ras(G12T), human H-Ras(G12T), or human N-Ras(G12T)) protein-GDP complex at least 2-fold stronger than the compound binds a human Ras(G12T) (e.g., human K-Ras(G12T), human H-Ras(G12T), or human N-Ras(G12T)) protein-GTP complex under identical conditions. In embodiments, the compound binds a human Ras(G12T) (e.g., human K-Ras(G12T), human H-Ras(G12T), or human N-Ras(G12T)) protein-GDP complex at least 5-fold stronger than the compound binds a human Ras(G12T) (e.g., human K-Ras(G12T), human H-Ras(G12T), or human N-Ras(G12T)) protein-GTP complex under identical conditions. In embodiments, the compound binds a human Ras(G12T) (e.g., human K-Ras(G12T), human H-Ras(G12T), or human N-Ras(G12T)) protein-GDP complex at least 10-fold stronger than the compound binds a human Ras(G12T) (e.g., human K-Ras(G12T), human H-Ras(G12T), or human N-Ras(G12T)) protein-GTP complex under identical conditions. In embodiments, the compound binds a human Ras(G12T) (e.g., human K-Ras(G12T), human H-Ras(G12T), or human N-Ras(G12T)) protein-GDP complex at least 20-fold stronger than the compound binds a human Ras(G12T) (e.g., human K-Ras(G12T), human H-Ras(G12T), or human N-Ras(G12T)) protein-GTP complex under identical conditions. In embodiments, the compound binds a human Ras(G12T) (e.g., human K-Ras(G12T), human H-Ras(G12T), or human N-Ras(G12T)) protein-GDP complex at least 40-fold stronger than said compound binds a human Ras(G12T) (e.g., human K-Ras(G12T), human H-Ras(G12T), or human N-Ras(G12T)) protein-GTP complex under identical conditions. In embodiments, the compound binds a human Ras(G12T) (e.g., human K-Ras(G12T), human H-Ras(G12T), or human N-Ras(G12T)) protein-GDP complex at least 60-fold stronger than the compound binds a human Ras(G12T) (e.g., human K-Ras(G12T), human H-Ras(G12T), or human N-Ras(G12T)) protein-GTP complex under identical conditions. In embodiments, the compound binds a human Ras(G12T) (e.g., human K-Ras(G12T), human H-Ras(G12T), or human N-Ras(G12T)) protein-GDP complex at least 80-fold stronger than the compound binds a human Ras(G12T) (e.g., human K-Ras(G12T), human H-Ras(G12T), or human N-Ras(G12T)) protein-GTP complex under identical conditions. In embodiments, the compound binds a human Ras(G12T) (e.g., human K-Ras(G12T), human H-Ras(G12T), or human N-Ras(G12T)) protein-GDP complex at least 100-fold stronger than said compound binds a human Ras(G12T) (e.g., human K-Ras(G12T), human H-Ras(G12T), or human N-Ras(G12T)) protein-GTP complex under identical conditions. In embodiments, the compound binds a human Ras(G12T) (e.g., human K-Ras(G12T), human H-Ras(G12T), or human N-Ras(G12T)) protein-GDP complex at least 500-fold stronger than the compound binds a human Ras(G12T) (e.g., human K-Ras(G12T), human H-Ras(G12T), or human N-Ras(G12T)) protein-GTP complex under identical conditions.

In embodiments, the compound binds a human Ras(G12D) (e.g., human K-Ras(G12D), human H-Ras(G12D), or human N-Ras(G12D)) protein-GDP complex more strongly than the compound binds a human Ras(G12D) (e.g., human K-Ras(G12D), human H-Ras(G12D), or human N-Ras(G12D)) protein-GTP complex under identical conditions. In embodiments, the compound binds a human Ras(G12D) (e.g., human K-Ras(G12D), human H-Ras(G12D), or human N-Ras(G12D)) protein-GDP complex at least 2-fold stronger than the compound binds a human Ras(G12D) (e.g., human K-Ras(G12D), human H-Ras(G12D), or human N-Ras(G12D)) protein-GTP complex under identical conditions. In embodiments, the compound binds a human Ras(G12D) (e.g., human K-Ras(G12D), human H-Ras(G12D), or human N-Ras(G12D)) protein-GDP complex at least 5-fold stronger than the compound binds a human Ras(G12D) (e.g., human K-Ras(G12D), human H-Ras(G12D), or human N-Ras(G12D)) protein-GTP complex under identical conditions. In embodiments, the compound binds a human Ras(G12D) (e.g., human K-Ras(G12D), human H-Ras(G12D), or human N-Ras(G12D)) protein-GDP complex at least 10-fold stronger than the compound binds a human Ras(G12D) (e.g., human K-Ras(G12D), human H-Ras(G12D), or human N-Ras(G12D)) protein-GTP complex under identical conditions. In embodiments, the compound binds a human Ras(G12D) (e.g., human K-Ras(G12D), human H-Ras(G12D), or human N-Ras(G12D)) protein-GDP complex at least 20-fold stronger than the compound binds a human Ras(G12D) (e.g., human K-Ras(G12D), human H-Ras(G12D), or human N-Ras(G12D)) protein-GTP complex under identical conditions. In embodiments, the compound binds a human Ras(G12D) (e.g., human K-Ras(G12D), human H-Ras(G12D), or human N-Ras(G12D)) protein-GDP complex at least 40-fold stronger than said compound binds a human Ras(G12D) (e.g., human K-Ras(G12D), human H-Ras(G12D), or human N-Ras(G12D)) protein-GTP complex under identical conditions. In embodiments, the compound binds a human Ras(G12D) (e.g., human K-Ras(G12D), human H-Ras(G12D), or human N-Ras(G12D)) protein-GDP complex at least 60-fold stronger than the compound binds a human Ras(G12D) (e.g., human K-Ras(G12D), human H-Ras(G12D), or human N-Ras(G12D)) protein-GTP complex under identical conditions. In embodiments, the compound binds a human Ras(G12D) (e.g., human K-Ras(G12D), human H-Ras(G12D), or human N-Ras(G12D)) protein-GDP complex at least 80-fold stronger than the compound binds a human Ras(G12D) (e.g., human K-Ras(G12D), human H-Ras(G12D), or human N-Ras(G12D)) protein-GTP complex under identical conditions. In embodiments, the compound binds a human Ras(G12D) (e.g., human K-Ras(G12D), human H-Ras(G12D), or human N-Ras(G12D)) protein-GDP complex at least 100-fold stronger than said compound binds a human Ras(G12D) (e.g., human K-Ras(G12D), human H-Ras(G12D), or human N-Ras(G12D)) protein-GTP complex under identical conditions. In embodiments, the compound binds a human Ras(G12D) (e.g., human K-Ras(G12D), human H-Ras(G12D), or human N-Ras(G12D)) protein-GDP complex at least 500-fold stronger than the compound binds a human Ras(G12D) (e.g., human K-Ras(G12D), human H-Ras(G12D), or human N-Ras(G12D)) protein-GTP complex under identical conditions.

In embodiments, the compound binds a human Ras(G13D) (e.g., human K-Ras(G13D), human H-Ras(G13D), or human N-Ras(G13D)) protein-GDP complex more strongly than the compound binds a human Ras(G13D) (e.g., human K-Ras(G13D), human H-Ras(G13D), or human N-Ras(G13D)) protein-GTP complex under identical conditions. In embodiments, the compound binds a human Ras(G13D) (e.g., human K-Ras(G13D), human H-Ras(G13D), or human N-Ras(G13D)) protein-GDP complex at least 2-fold stronger than the compound binds a human Ras(G13D) (e.g., human K-Ras(G13D), human H-Ras(G13D), or human N-Ras(G13D)) protein-GTP complex under identical conditions. In embodiments, the compound binds a human Ras(G13D) (e.g., human K-Ras(G13D), human H-Ras(G13D), or human N-Ras(G13D)) protein-GDP complex at least 5-fold stronger than the compound binds a human Ras(G13D) (e.g., human K-Ras(G13D), human H-Ras(G13D), or human N-Ras(G13D)) protein-GTP complex under identical conditions. In embodiments, the compound binds a human Ras(G13D) (e.g., human K-Ras(G13D), human H-Ras(G13D), or human N-Ras(G13D)) protein-GDP complex at least 10-fold stronger than the compound binds a human Ras(G13D) (e.g., human K-Ras(G13D), human H-Ras(G13D), or human N-Ras(G13D)) protein-GTP complex under identical conditions. In embodiments, the compound binds a human Ras(G13D) (e.g., human K-Ras(G13D), human H-Ras(G13D), or human N-Ras(G13D)) protein-GDP complex at least 20-fold stronger than the compound binds a human Ras(G13D) (e.g., human K-Ras(G13D), human H-Ras(G13D), or human N-Ras(G13D)) protein-GTP complex under identical conditions. In embodiments, the compound binds a human Ras(G13D) (e.g., human K-Ras(G13D), human H-Ras(G13D), or human N-Ras(G13D)) protein-GDP complex at least 40-fold stronger than said compound binds a human Ras(G13D) (e.g., human K-Ras(G13D), human H-Ras(G13D), or human N-Ras(G13D)) protein-GTP complex under identical conditions. In embodiments, the compound binds a human Ras(G13D) (e.g., human K-Ras(G13D), human H-Ras(G13D), or human N-Ras(G13D)) protein-GDP complex at least 60-fold stronger than the compound binds a human Ras(G13D) (e.g., human K-Ras(G13D), human H-Ras(G13D), or human N-Ras(G13D)) protein-GTP complex under identical conditions. In embodiments, the compound binds a human Ras(G13D) (e.g., human K-Ras(G13D), human H-Ras(G13D), or human N-Ras(G13D)) protein-GDP complex at least 80-fold stronger than the compound binds a human Ras(G13D) (e.g., human K-Ras(G13D), human H-Ras(G13D), or human N-Ras(G13D)) protein-GTP complex under identical conditions. In embodiments, the compound binds a human Ras(G13D) (e.g., human K-Ras(G13D), human H-Ras(G13D), or human N-Ras(G13D)) protein-GDP complex at least 100-fold stronger than said compound binds a human Ras(G13D) (e.g., human K-Ras(G13D), human H-Ras(G13D), or human N-Ras(G13D)) protein-GTP complex under identical conditions. In embodiments, the compound binds a human Ras(G13D) (e.g., human K-Ras(G13D), human H-Ras(G13D), or human N-Ras(G13D)) protein-GDP complex at least 500-fold stronger than the compound binds a human Ras(G13D) (e.g., human K-Ras(G13D), human H-Ras(G13D), or human N-Ras(G13D)) protein-GTP complex under identical conditions.

In embodiments, the compound binds a human Ras(G12E) (e.g., human K-Ras(G12E), human H-Ras(G12E), or human N-Ras(G12E)) protein-GDP complex more strongly than the compound binds a human Ras(G12E) (e.g., human K-Ras(G12E), human H-Ras(G12E), or human N-Ras(G12E)) protein-GTP complex under identical conditions. In embodiments, the compound binds a human Ras(G12E) (e.g., human K-Ras(G12E), human H-Ras(G12E), or human N-Ras(G12E)) protein-GDP complex at least 2-fold stronger than the compound binds a human Ras(G12E) (e.g., human K-Ras(G12E), human H-Ras(G12E), or human N-Ras(G12E)) protein-GTP complex under identical conditions. In embodiments, the compound binds a human Ras(G12E) (e.g., human K-Ras(G12E), human H-Ras(G12E), or human N-Ras(G12E)) protein-GDP complex at least 5-fold stronger than the compound binds a human Ras(G12E) (e.g., human K-Ras(G12E), human H-Ras(G12E), or human N-Ras(G12E)) protein-GTP complex under identical conditions. In embodiments, the compound binds a human Ras(G12E) (e.g., human K-Ras(G12E), human H-Ras(G12E), or human N-Ras(G12E)) protein-GDP complex at least 10-fold stronger than the compound binds a human Ras(G12E) (e.g., human K-Ras(G12E), human H-Ras(G12E), or human N-Ras(G12E)) protein-GTP complex under identical conditions. In embodiments, the compound binds a human Ras(G12E) (e.g., human K-Ras(G12E), human H-Ras(G12E), or human N-Ras(G12E)) protein-GDP complex at least 20-fold stronger than the compound binds a human Ras(G12E) (e.g., human K-Ras(G12E), human H-Ras(G12E), or human N-Ras(G12E)) protein-GTP complex under identical conditions. In embodiments, the compound binds a human Ras(G12E) (e.g., human K-Ras(G12E), human H-Ras(G12E), or human N-Ras(G12E)) protein-GDP complex at least 40-fold stronger than said compound binds a human Ras(G12E) (e.g., human K-Ras(G12E), human H-Ras(G12E), or human N-Ras(G12E)) protein-GTP complex under identical conditions. In embodiments, the compound binds a human Ras(G12E) (e.g., human K-Ras(G12E), human H-Ras(G12E), or human N-Ras(G12E)) protein-GDP complex at least 60-fold stronger than the compound binds a human Ras(G12E) (e.g., human K-Ras(G12E), human H-Ras(G12E), or human N-Ras(G12E)) protein-GTP complex under identical conditions. In embodiments, the compound binds a human Ras(G12E) (e.g., human K-Ras(G12E), human H-Ras(G12E), or human N-Ras(G12E)) protein-GDP complex at least 80-fold stronger than the compound binds a human Ras(G12E) (e.g., human K-Ras(G12E), human H-Ras(G12E), or human N-Ras(G12E)) protein-GTP complex under identical conditions. In embodiments, the compound binds a human Ras(G12E) (e.g., human K-Ras(G12E), human H-Ras(G12E), or human N-Ras(G12E)) protein-GDP complex at least 100-fold stronger than said compound binds a human Ras(G12E) (e.g., human K-Ras(G12E), human H-Ras(G12E), or human N-Ras(G12E)) protein-GTP complex under identical conditions. In embodiments, the compound binds a human Ras(G12E) (e.g., human K-Ras(G12E), human H-Ras(G12E), or human N-Ras(G12E)) protein-GDP complex at least 500-fold stronger than the compound binds a human Ras(G12E) (e.g., human K-Ras(G12E), human H-Ras(G12E), or human N-Ras(G12E)) protein-GTP complex under identical conditions.

In embodiments, the compound is useful as a comparator compound. In embodiments, the comparator compound can be used to assess the activity of a test compound as set forth in an assay described herein (e.g., in the examples section, figures, or tables).

In embodiments, the compound is a compound as described herein, including in embodiments. In embodiments the compound is a compound described herein (e.g., in the examples section, figures, tables, or claims).

### III. Pharmaceutical Compositions

In an aspect is provided a pharmaceutical composition including a compound described herein, or a pharmaceutically acceptable salt thereof, and a pharmaceutically acceptable excipient.

In embodiments, the pharmaceutical composition includes an effective amount of the compound. In embodiments, the pharmaceutical composition includes a therapeutically effective amount of the compound. In embodiments, the compound is a compound of formula (I), (I-1), (I-1a), (I-1b), (I-1c), (I-1d), (I-1e), (I-1f), (I-2), (I-2a), (I-2b), (I-3), (I-3a), (I-3b), (I-3c), (I-4), (I-4a), (I-4b), (I-5), (I-5a), (I-5b), (I-6), (I-6a), (I-7), (I-7a), (II), (II-1), (II-1a), (III), (III-1), (III-1a), (III-2), or (III-2a). In embodiments, the compound is a compound of formula (IV), (IV-1), (IV-1a), (IV-1b), (IV-1c), (IV-1d), (IV-1e), (IV-1f), (IV-2), (IV-2a), (IV-2b), (IV-3), (IV-3a), (IV-3b), (IV-3c), (IV-4), (IV-4a), (IV-4b), (IV-5), (IV-5a), (IV-5b), (IV-6), (IV-6a), (IV-6b), (IV-7), (IV-7a), (IV-7b), (IV-8), (IV-8a), (IV-8b), (IV-9), (IV-9a), (IV-9b), (IV-9c), (IV-9d), (IV-10), (IV-10a), (IV-11), (IV-11a), (V), (V-1), (V-1a), (VI), (VI-1), (VI-1a), (VI-2), or (VI-2a).

### IV. Methods of Use

In an aspect is provided a method of treating cancer in a subject in need thereof, the method including administering to the subject in need thereof a therapeutically effective amount of a compound described herein, or a pharmaceutically acceptable salt thereof.

In embodiments, the cancer is rectal carcinoma, colorectal adenocarcinoma, colorectal carcinoma, non-small cell lung carcinoma, squamous cell lung carcinoma, myelodysplastic syndrome, or acute myeloid leukemia.

In an aspect is provided a method of treating a Switch II GTPase protein-associated disease in a subject in need thereof, the method including administering to the subject in need thereof a therapeutically effective amount of a compound described herein, or a pharmaceutically acceptable salt thereof. In embodiments, the Switch II GTPase protein-associated disease is cancer (e.g., rectal carcinoma, colorectal adenocarcinoma, colorectal carcinoma, non-small cell lung carcinoma, squamous cell lung carcinoma, myelodysplastic syndrome, or acute myeloid leukemia). In embodiments, the Switch II GTPase protein-associated disease is a RASopathy (e.g., capillary malformation-AV malformation syndrome, autoimmune lymphoproliferative syndrome, cardiofaciocutaneous syndrome, hereditary gingival fibromatosis type 1, neurofibromatosis type 1, Noonan syndrome, Costello syndrome, or Legius syndrome).

In an aspect is provided a method of treating a K-Ras(G12S)-associated disease in a subject in need thereof, the method including administering to the subject in need thereof a therapeutically effective amount of a compound described herein, or a pharmaceutically acceptable salt thereof. In embodiments, the K-Ras(G12S)-associated disease is cancer (e.g., rectal carcinoma, colorectal adenocarcinoma, colorectal carcinoma, non-small cell lung carcinoma, squamous cell lung carcinoma, myelodysplastic syndrome, or acute myeloid leukemia). In embodiments, the K-Ras(G12S)-associated disease is a RASopathy (e.g., capillary malformation-AV malformation syndrome, autoimmune lymphoproliferative syndrome, cardiofaciocutaneous syndrome, hereditary gingival fibromatosis type 1, neurofibromatosis type 1, Noonan syndrome, Costello syndrome, or Legius syndrome).

In an aspect is provided a method of treating an H-Ras(G12S)-associated disease in a subject in need thereof, the method including administering to the subject in need thereof a therapeutically effective amount of a compound described herein, or a pharmaceutically acceptable salt thereof. In embodiments, the H-Ras(G12S)-associated disease is cancer (e.g., rectal carcinoma, colorectal adenocarcinoma, colorectal carcinoma, non-small cell lung carcinoma, squamous cell lung carcinoma, myelodysplastic syndrome, or acute myeloid leukemia). In embodiments, the H-Ras(G12S)-associated disease is a RASopathy (e.g., capillary malformation-AV malformation syndrome, autoimmune lymphoproliferative syndrome, cardiofaciocutaneous syndrome, hereditary gingival fibromatosis type 1, neurofibromatosis type 1, Noonan syndrome, Costello syndrome, or Legius syndrome).

In embodiments, the H-Ras(G12S)-associated disease is Costello syndrome.

In an aspect is provided a method of treating an N-Ras(G12S)-associated disease in a subject in need thereof, the method including administering to the subject in need thereof a therapeutically effective amount of a compound described herein, or a pharmaceutically acceptable salt thereof. In embodiments, the N-Ras(G12S)-associated disease is cancer (e.g., rectal carcinoma, colorectal adenocarcinoma, colorectal carcinoma, non-small cell lung carcinoma, squamous cell lung carcinoma, myelodysplastic syndrome, or acute myeloid leukemia). In embodiments, the N-Ras(G12S)-associated disease is a RASopathy (e.g., capillary malformation-AV malformation syndrome, autoimmune lymphoproliferative syndrome, cardiofaciocutaneous syndrome, hereditary gingival fibromatosis type 1, neurofibromatosis type 1, Noonan syndrome, Costello syndrome, or Legius syndrome).

In an aspect is provided a method of treating a K-Ras(G13S)-associated disease in a subject in need thereof, the method including administering to the subject in need thereof a therapeutically effective amount of a compound described herein, or a pharmaceutically acceptable salt thereof. In embodiments, the K-Ras(G13S)-associated disease is cancer (e.g., rectal carcinoma, colorectal adenocarcinoma, colorectal carcinoma, non-small cell lung carcinoma, squamous cell lung carcinoma, myelodysplastic syndrome, or acute myeloid leukemia). In embodiments, the K-Ras(G13S)-associated disease is a RASopathy (e.g., capillary malformation-AV malformation syndrome, autoimmune lymphoproliferative syndrome, cardiofaciocutaneous syndrome, hereditary gingival fibromatosis type 1, neurofibromatosis type 1, Noonan syndrome, Costello syndrome, or Legius syndrome).

In an aspect is provided a method of treating an H-Ras(G13S)-associated disease in a subject in need thereof, the method including administering to the subject in need thereof a therapeutically effective amount of a compound described herein, or a pharmaceutically acceptable salt thereof. In embodiments, the H-Ras(G13S)-associated disease is cancer (e.g., rectal carcinoma, colorectal adenocarcinoma, colorectal carcinoma, non-small cell lung carcinoma, squamous cell lung carcinoma, myelodysplastic syndrome, or acute myeloid leukemia). In embodiments, the H-Ras(G13S)-associated disease is a RASopathy (e.g., capillary malformation-AV malformation syndrome, autoimmune lymphoproliferative syndrome, cardiofaciocutaneous syndrome, hereditary gingival fibromatosis type 1, neurofibromatosis type 1, Noonan syndrome, Costello syndrome, or Legius syndrome).

In an aspect is provided a method of treating an N-Ras(G13S)-associated disease in a subject in need thereof, the method including administering to the subject in need thereof a therapeutically effective amount of a compound described herein, or a pharmaceutically acceptable salt thereof. In embodiments, the N-Ras(G13S)-associated disease is cancer (e.g., rectal carcinoma, colorectal adenocarcinoma, colorectal carcinoma, non-small cell lung carcinoma, squamous cell lung carcinoma, myelodysplastic syndrome, or acute myeloid leukemia). In embodiments, the N-Ras(G13S)-associated disease is a RASopathy (e.g., capillary malformation-AV malformation syndrome, autoimmune lymphoproliferative syndrome, cardiofaciocutaneous syndrome, hereditary gingival fibromatosis type 1, neurofibromatosis type 1, Noonan syndrome, Costello syndrome, or Legius syndrome).

In an aspect is provided a method of treating a K-Ras(G12T)-associated disease in a subject in need thereof, the method including administering to the subject in need thereof a therapeutically effective amount of a compound described herein, or a pharmaceutically acceptable salt thereof. In embodiments, the K-Ras(G12T)-associated disease is cancer (e.g., rectal carcinoma, colorectal adenocarcinoma, colorectal carcinoma, non-small cell lung carcinoma, squamous cell lung carcinoma, myelodysplastic syndrome, or acute myeloid leukemia). In embodiments, the K-Ras(G12T)-associated disease is a RASopathy (e.g., capillary malformation-AV malformation syndrome, autoimmune lymphoproliferative syndrome, cardiofaciocutaneous syndrome, hereditary gingival fibromatosis type 1, neurofibromatosis type 1, Noonan syndrome, Costello syndrome, or Legius syndrome).

In an aspect is provided a method of treating an H-Ras(G12T)-associated disease in a subject in need thereof, the method including administering to the subject in need thereof a therapeutically effective amount of a compound described herein, or a pharmaceutically acceptable salt thereof. In embodiments, the H-Ras(G12T)-associated disease is cancer (e.g., rectal carcinoma, colorectal adenocarcinoma, colorectal carcinoma, non-small cell lung carcinoma, squamous cell lung carcinoma, myelodysplastic syndrome, or acute myeloid leukemia). In embodiments, the H-Ras(G12T)-associated disease is a RASopathy (e.g., capillary malformation-AV malformation syndrome, autoimmune lymphoproliferative syndrome, cardiofaciocutaneous syndrome, hereditary gingival fibromatosis type 1, neurofibromatosis type 1, Noonan syndrome, Costello syndrome, or Legius syndrome).

In an aspect is provided a method of treating an N-Ras(G12T)-associated disease in a subject in need thereof, the method including administering to the subject in need thereof a therapeutically effective amount of a compound described herein, or a pharmaceutically acceptable salt thereof. In embodiments, the N-Ras(G12T)-associated disease is cancer (e.g., rectal carcinoma, colorectal adenocarcinoma, colorectal carcinoma, non-small cell lung carcinoma, squamous cell lung carcinoma, myelodysplastic syndrome, or acute myeloid leukemia). In embodiments, the N-Ras(G12T)-associated disease is a RASopathy (e.g., capillary malformation-AV malformation syndrome, autoimmune lymphoproliferative syndrome, cardiofaciocutaneous syndrome, hereditary gingival fibromatosis type 1, neurofibromatosis type 1, Noonan syndrome, Costello syndrome, or Legius syndrome).

In an aspect is provided a method of treating a K-Ras(G12D)-associated disease in a subject in need thereof, the method including administering to the subject in need thereof a therapeutically effective amount of a compound described herein, or a pharmaceutically acceptable salt thereof. In embodiments, the K-Ras(G12D)-associated disease is cancer (e.g., rectal carcinoma, colorectal adenocarcinoma, colorectal carcinoma, non-small cell lung carcinoma, squamous cell lung carcinoma, myelodysplastic syndrome, or acute myeloid leukemia). In embodiments, the K-Ras(G12D)-associated disease is a RASopathy (e.g., capillary malformation-AV malformation syndrome, autoimmune lymphoproliferative syndrome, cardiofaciocutaneous syndrome, hereditary gingival fibromatosis type 1, neurofibromatosis type 1, Noonan syndrome, Costello syndrome, or Legius syndrome).

In an aspect is provided a method of treating an H-Ras(G12D)-associated disease in a subject in need thereof, the method including administering to the subject in need thereof a therapeutically effective amount of a compound described herein, or a pharmaceutically acceptable salt thereof. In embodiments, the H-Ras(G12D)-associated disease is cancer (e.g., rectal carcinoma, colorectal adenocarcinoma, colorectal carcinoma, non-small cell lung carcinoma, squamous cell lung carcinoma, myelodysplastic syndrome, or acute myeloid leukemia). In embodiments, the H-Ras(G12D)-associated disease is a RASopathy (e.g., capillary malformation-AV malformation syndrome, autoimmune lymphoproliferative syndrome, cardiofaciocutaneous syndrome, hereditary gingival fibromatosis type 1, neurofibromatosis type 1, Noonan syndrome, Costello syndrome, or Legius syndrome).

In an aspect is provided a method of treating an N-Ras(G12D)-associated disease in a subject in need thereof, the method including administering to the subject in need thereof a therapeutically effective amount of a compound described herein, or a pharmaceutically acceptable salt thereof. In embodiments, the N-Ras(G12D)-associated disease is cancer (e.g., rectal carcinoma, colorectal adenocarcinoma, colorectal carcinoma, non-small cell lung carcinoma, squamous cell lung carcinoma, myelodysplastic syndrome, or acute myeloid leukemia). In embodiments, the N-Ras(G12D)-associated disease is a RASopathy (e.g., capillary malformation-AV malformation syndrome, autoimmune lymphoproliferative syndrome, cardiofaciocutaneous syndrome, hereditary gingival fibromatosis type 1, neurofibromatosis type 1, Noonan syndrome, Costello syndrome, or Legius syndrome).

In an aspect is provided a method of treating a K-Ras(G13D)-associated disease in a subject in need thereof, the method including administering to the subject in need thereof a therapeutically effective amount of a compound described herein, or a pharmaceutically acceptable salt thereof. In embodiments, the K-Ras(G13D)-associated disease is cancer (e.g., rectal carcinoma, colorectal adenocarcinoma, colorectal carcinoma, non-small cell lung carcinoma, squamous cell lung carcinoma, myelodysplastic syndrome, or acute myeloid leukemia). In embodiments, the K-Ras(G13D)-associated disease is a RASopathy (e.g., capillary malformation-AV malformation syndrome, autoimmune lymphoproliferative syndrome, cardiofaciocutaneous syndrome, hereditary gingival fibromatosis type 1, neurofibromatosis type 1, Noonan syndrome, Costello syndrome, or Legius syndrome).

In an aspect is provided a method of treating an H-Ras(G13D)-associated disease in a subject in need thereof, the method including administering to the subject in need thereof a therapeutically effective amount of a compound described herein, or a pharmaceutically acceptable salt thereof. In embodiments, the H-Ras(G13D)-associated disease is cancer (e.g., rectal carcinoma, colorectal adenocarcinoma, colorectal carcinoma, non-small cell lung carcinoma, squamous cell lung carcinoma, myelodysplastic syndrome, or acute myeloid leukemia). In embodiments, the H-Ras(G13D)-associated disease is a RASopathy (e.g., capillary malformation-AV malformation syndrome, autoimmune lymphoproliferative syndrome, cardiofaciocutaneous syndrome, hereditary gingival fibromatosis type 1, neurofibromatosis type 1, Noonan syndrome, Costello syndrome, or Legius syndrome).

In an aspect is provided a method of treating an N-Ras(G13D)-associated disease in a subject in need thereof, the method including administering to the subject in need thereof a therapeutically effective amount of a compound described herein, or a pharmaceutically acceptable salt thereof. In embodiments, the N-Ras(G13D)-associated disease is cancer (e.g., rectal carcinoma, colorectal adenocarcinoma, colorectal carcinoma, non-small cell lung carcinoma, squamous cell lung carcinoma, myelodysplastic syndrome, or acute myeloid leukemia). In embodiments, the N-Ras(G13D)-associated disease is a RASopathy (e.g., capillary malformation-AV malformation syndrome, autoimmune lymphoproliferative syndrome, cardiofaciocutaneous syndrome, hereditary gingival fibromatosis type 1, neurofibromatosis type 1, Noonan syndrome, Costello syndrome, or Legius syndrome).

In an aspect is provided a method of treating a K-Ras(G12E)-associated disease in a subject in need thereof, the method including administering to the subject in need thereof a therapeutically effective amount of a compound described herein, or a pharmaceutically acceptable salt thereof. In embodiments, the K-Ras(G12E)-associated disease is cancer (e.g., rectal carcinoma, colorectal adenocarcinoma, colorectal carcinoma, non-small cell lung carcinoma, squamous cell lung carcinoma, myelodysplastic syndrome, or acute myeloid leukemia). In embodiments, the K-Ras(G12E)-associated disease is a RASopathy (e.g., capillary malformation-AV malformation syndrome, autoimmune lymphoproliferative syndrome, cardiofaciocutaneous syndrome, hereditary gingival fibromatosis type 1, neurofibromatosis type 1, Noonan syndrome, Costello syndrome, or Legius syndrome).

In an aspect is provided a method of treating an H-Ras(G12E)-associated disease in a subject in need thereof, the method including administering to the subject in need thereof a therapeutically effective amount of a compound described herein, or a pharmaceutically acceptable salt thereof. In embodiments, the H-Ras(G12E)-associated disease is cancer (e.g., rectal carcinoma, colorectal adenocarcinoma, colorectal carcinoma, non-small cell lung carcinoma, squamous cell lung carcinoma, myelodysplastic syndrome, or acute myeloid leukemia). In embodiments, the H-Ras(G12E)-associated disease is a RASopathy (e.g., capillary malformation-AV malformation syndrome, autoimmune lymphoproliferative syndrome, cardiofaciocutaneous syndrome, hereditary gingival fibromatosis type 1, neurofibromatosis type 1, Noonan syndrome, Costello syndrome, or Legius syndrome).

In an aspect is provided a method of treating an N-Ras(G12E)-associated disease in a subject in need thereof, the method including administering to the subject in need thereof a therapeutically effective amount of a compound described herein, or a pharmaceutically acceptable salt thereof. In embodiments, the N-Ras(G12E)-associated disease is cancer (e.g., rectal carcinoma, colorectal adenocarcinoma, colorectal carcinoma, non-small cell lung carcinoma, squamous cell lung carcinoma, myelodysplastic syndrome, or acute myeloid leukemia). In embodiments, the N-Ras(G12E)-associated disease is a RASopathy (e.g., capillary malformation-AV malformation syndrome, autoimmune lymphoproliferative syndrome, cardiofaciocutaneous syndrome, hereditary gingival fibromatosis type 1, neurofibromatosis type 1, Noonan syndrome, Costello syndrome, or Legius syndrome).

In an aspect is provided a method of modulating the level of activity of a GTPase protein in a cell, the method including contacting the cell with an effective amount of a compound described herein, or a pharmaceutically acceptable salt thereof.

In an aspect is provided a method of modulating the level of activity of a K-Ras protein in a cell, the method including contacting the cell with an effective amount of a compound described herein, or a pharmaceutically acceptable salt thereof.

In embodiments, the modulating of the activity includes modulating GTPase activity, nucleotide exchange, differential GDP or GTP binding, effector protein binding, effector protein activation, guanine exchange factor (GEF) binding, GEF-facilitated nucleotide exchange, phosphate release, nucleotide release, nucleotide binding, K-Ras subcellular localization, K-Ras post-translational processing, or K-Ras post-translational modifications.

In embodiments, the modulating is increasing the activity of the K-Ras protein. In embodiments, the level of activity of the K-Ras protein is increased by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold relative to a control (e.g., absence of the compound). In embodiments, the level of activity of the K-Ras protein is increased by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold relative to a control (e.g., absence of the compound).

In embodiments, the level of activity of the K-Ras protein is increased by about 1.5-fold relative to a control (e.g., absence of the compound). In embodiments, the level of activity of the K-Ras protein is increased by about 2-fold relative to a control (e.g., absence of the compound). In embodiments, the level of activity of the K-Ras protein is increased by about 5-fold relative to a control (e.g., absence of the compound). In embodiments, the level of activity of the K-Ras protein is increased by about 10-fold relative to a control (e.g., absence of the compound). In embodiments, the level of activity of the K-Ras protein is increased by about 25-fold relative to a control (e.g., absence of the compound). In embodiments, the level of activity of the K-Ras protein is increased by about 50-fold relative to a control (e.g., absence of the compound). In embodiments, the level of activity of the K-Ras protein is increased by about 100-fold relative to a control (e.g., absence of the compound). In embodiments, the level of activity of the K-Ras protein is increased by about 500-fold relative to a control (e.g., absence of the compound). In embodiments, the level of activity of the K-Ras protein is increased by about 1000-fold relative to a control (e.g., absence of the compound).

In embodiments, the level of activity of the K-Ras protein is increased by at least 1.5-fold relative to a control (e.g., absence of the compound). In embodiments, the level of activity of the K-Ras protein is increased by at least 2-fold relative to a control (e.g., absence of the compound). In embodiments, the level of activity of the K-Ras protein is increased by at least 5-fold relative to a control (e.g., absence of the compound). In embodiments, the level of activity of the K-Ras protein is increased by at least 10-fold relative to a control (e.g., absence of the compound). In embodiments, the level of activity of the K-Ras protein is increased by at least 25-fold relative to a control (e.g., absence of the compound). In embodiments, the level of activity of the K-Ras protein is increased by at least 50-fold relative to a control (e.g., absence of the compound). In embodiments, the level of activity of the K-Ras protein is increased by at least 100-fold relative to a control (e.g., absence of the compound). In embodiments, the level of activity of the K-Ras protein is increased by at least 500-fold relative to a control (e.g., absence of the compound). In embodiments, the level of activity of the K-Ras protein is increased by at least 1000-fold relative to a control (e.g., absence of the compound).

In embodiments, the modulating is reducing the activity of the K-Ras protein. In embodiments, the level of activity of the K-Ras protein is reduced by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold relative to a control (e.g., absence of the compound). In embodiments, the level of activity of the K-Ras protein is reduced by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold relative to a control (e.g., absence of the compound).

In embodiments, the level of activity of the K-Ras protein is reduced by about 1.5-fold relative to a control (e.g., absence of the compound). In embodiments, the level of activity of the K-Ras protein is reduced by about 2-fold relative to a control (e.g., absence of the compound). In embodiments, the level of activity of the K-Ras protein is reduced by about 5-fold relative to a control (e.g., absence of the compound). In embodiments, the level of activity of the K-Ras protein is reduced by about 10-fold relative to a control (e.g., absence of the compound). In embodiments, the level of activity of the K-Ras protein is reduced by about 25-fold relative to a control (e.g., absence of the compound). In embodiments, the level of activity of the K-Ras protein is reduced by about 50-fold relative to a control (e.g., absence of the compound). In embodiments, the level of activity of the K-Ras protein is reduced by about 100-fold relative to a control (e.g., absence of the compound). In embodiments, the level of activity of the K-Ras protein is reduced by about 500-fold relative to a control (e.g., absence of the compound). In embodiments, the level of activity of the K-Ras protein is reduced by about 1000-fold relative to a control (e.g., absence of the compound).

In embodiments, the level of activity of the K-Ras protein is reduced by at least 1.5-fold relative to a control (e.g., absence of the compound). In embodiments, the level of activity of the K-Ras protein is reduced by at least 2-fold relative to a control (e.g., absence of the compound). In embodiments, the level of activity of the K-Ras protein is reduced by at least 5-fold relative to a control (e.g., absence of the compound). In embodiments, the level of activity of the K-Ras protein is reduced by at least 10-fold relative to a control (e.g., absence of the compound). In embodiments, the level of activity of the K-Ras protein is reduced by at least 25-fold relative to a control (e.g., absence of the compound). In embodiments, the level of activity of the K-Ras protein is reduced by at least 50-fold relative to a control (e.g., absence of the compound). In embodiments, the level of activity of the K-Ras protein is reduced by at least 100-fold relative to a control (e.g., absence of the compound). In embodiments, the level of activity of the K-Ras protein is reduced by at least 500-fold relative to a control (e.g., absence of the compound). In embodiments, the level of activity of the K-Ras protein is reduced by at least 1000-fold relative to a control (e.g., absence of the compound).

In embodiments, the K-Ras protein is a human K-Ras protein. In embodiments, the human K-Ras protein contains a G12S mutation. In embodiments, the human K-Ras protein contains a G13S mutation. In embodiments, the human K-Ras protein contains a G12T mutation.

In embodiments, the K-Ras protein is a human K-Ras protein. In embodiments, the human K-Ras protein contains a G12D mutation. In embodiments, the human K-Ras protein contains a G13D mutation. In embodiments, the human K-Ras protein contains a G12E mutation.

In an aspect is provided a method of modulating the level of activity of an H-Ras protein in a cell, the method including contacting the cell with an effective amount of a compound described herein, or a pharmaceutically acceptable salt thereof.

In embodiments, the modulating of the activity includes modulating GTPase activity, nucleotide exchange, differential GDP or GTP binding, effector protein binding, effector protein activation, guanine exchange factor (GEF) binding, GEF-facilitated nucleotide exchange, phosphate release, nucleotide release, nucleotide binding, H-Ras subcellular localization, H-Ras post-translational processing, or H-Ras post-translational modifications.

In embodiments, the modulating is increasing the activity of the H-Ras protein. In embodiments, the level of activity of the H-Ras protein is increased by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold relative to a control (e.g., absence of the compound). In embodiments, the level of activity of the H-Ras protein is increased by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold relative to a control (e.g., absence of the compound).

In embodiments, the level of activity of the H-Ras protein is increased by about 1.5-fold relative to a control (e.g., absence of the compound). In embodiments, the level of activity of the H-Ras protein is increased by about 2-fold relative to a control (e.g., absence of the compound). In embodiments, the level of activity of the H-Ras protein is increased by about 5-fold relative to a control (e.g., absence of the compound). In embodiments, the level of activity of the H-Ras protein is increased by about 10-fold relative to a control (e.g., absence of the compound). In embodiments, the level of activity of the H-Ras protein is increased by about 25-fold relative to a control (e.g., absence of the compound). In embodiments, the level of activity of the H-Ras protein is increased by about 50-fold relative to a control (e.g., absence of the compound). In embodiments, the level of activity of the H-Ras protein is increased by about 100-fold relative to a control (e.g., absence of the compound). In embodiments, the level of activity of the H-Ras protein is increased by about 500-fold relative to a control (e.g., absence of the compound). In embodiments, the level of activity of the H-Ras protein is increased by about 1000-fold relative to a control (e.g., absence of the compound).

In embodiments, the level of activity of the H-Ras protein is increased by at least 1.5-fold relative to a control (e.g., absence of the compound). In embodiments, the level of activity of the H-Ras protein is increased by at least 2-fold relative to a control (e.g., absence of the compound). In embodiments, the level of activity of the H-Ras protein is increased by at least 5-fold relative to a control (e.g., absence of the compound). In embodiments, the level of activity of the H-Ras protein is increased by at least 10-fold relative to a control (e.g., absence of the compound). In embodiments, the level of activity of the H-Ras protein is increased by at least 25-fold relative to a control (e.g., absence of the compound). In embodiments, the level of activity of the H-Ras protein is increased by at least 50-fold relative to a control (e.g., absence of the compound). In embodiments, the level of activity of the H-Ras protein is increased by at least 100-fold relative to a control (e.g., absence of the compound). In embodiments, the level of activity of the H-Ras protein is increased by at least 500-fold relative to a control (e.g., absence of the compound). In embodiments, the level of activity of the H-Ras protein is increased by at least 1000-fold relative to a control (e.g., absence of the compound).

In embodiments, the modulating is reducing the activity of the H-Ras protein. In embodiments, the level of activity of the H-Ras protein is reduced by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold relative to a control (e.g., absence of the compound). In embodiments, the level of activity of the H-Ras protein is reduced by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold relative to a control (e.g., absence of the compound).

In embodiments, the level of activity of the H-Ras protein is reduced by about 1.5-fold relative to a control (e.g., absence of the compound). In embodiments, the level of activity of the H-Ras protein is reduced by about 2-fold relative to a control (e.g., absence of the compound). In embodiments, the level of activity of the H-Ras protein is reduced by about 5-fold relative to a control (e.g., absence of the compound). In embodiments, the level of activity of the H-Ras protein is reduced by about 10-fold relative to a control (e.g., absence of the compound). In embodiments, the level of activity of the H-Ras protein is reduced by about 25-fold relative to a control (e.g., absence of the compound). In embodiments, the level of activity of the H-Ras protein is reduced by about 50-fold relative to a control (e.g., absence of the compound). In embodiments, the level of activity of the H-Ras protein is reduced by about 100-fold relative to a control (e.g., absence of the compound). In embodiments, the level of activity of the H-Ras protein is reduced by about 500-fold relative to a control (e.g., absence of the compound). In embodiments, the level of activity of the H-Ras protein is reduced by about 1000-fold relative to a control (e.g., absence of the compound).

In embodiments, the level of activity of the H-Ras protein is reduced by at least 1.5-fold relative to a control (e.g., absence of the compound). In embodiments, the level of activity of the H-Ras protein is reduced by at least 2-fold relative to a control (e.g., absence of the compound). In embodiments, the level of activity of the H-Ras protein is reduced by at least 5-fold relative to a control (e.g., absence of the compound). In embodiments, the level of activity of the H-Ras protein is reduced by at least 10-fold relative to a control (e.g., absence of the compound). In embodiments, the level of activity of the H-Ras protein is reduced by at least 25-fold relative to a control (e.g., absence of the compound). In embodiments, the level of activity of the H-Ras protein is reduced by at least 50-fold relative to a control (e.g., absence of the compound). In embodiments, the level of activity of the H-Ras protein is reduced by at least 100-fold relative to a control (e.g., absence of the compound). In embodiments, the level of activity of the H-Ras protein is reduced by at least 500-fold relative to a control (e.g., absence of the compound). In embodiments, the level of activity of the H-Ras protein is reduced by at least 1000-fold relative to a control (e.g., absence of the compound).

In embodiments, the H-Ras protein is a human H-Ras protein. In embodiments, the human H-Ras protein contains a G12S mutation. In embodiments, the human H-Ras protein contains a G13S mutation. In embodiments, the human H-Ras protein contains a G12T mutation.

In embodiments, the H-Ras protein is a human H-Ras protein. In embodiments, the human H-Ras protein contains a G12D mutation. In embodiments, the human H-Ras protein contains a G13D mutation. In embodiments, the human H-Ras protein contains a G12E mutation.

In an aspect is provided a method of modulating the level of activity of an N-Ras protein in a cell, the method including contacting the cell with an effective amount of a compound described herein, or a pharmaceutically acceptable salt thereof.

In embodiments, the modulating of the activity includes modulating GTPase activity, nucleotide exchange, differential GDP or GTP binding, effector protein binding, effector protein activation, guanine exchange factor (GEF) binding, GEF-facilitated nucleotide exchange, phosphate release, nucleotide release, nucleotide binding, N-Ras subcellular localization, N-Ras post-translational processing, or N-Ras post-translational modifications.

In embodiments, the modulating is increasing the activity of the N-Ras protein. In embodiments, the level of activity of the N-Ras protein is increased by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold relative to a control (e.g., absence of the compound). In embodiments, the level of activity of the N-Ras protein is increased by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold relative to a control (e.g., absence of the compound).

In embodiments, the level of activity of the N-Ras protein is increased by about 1.5-fold relative to a control (e.g., absence of the compound). In embodiments, the level of activity of the N-Ras protein is increased by about 2-fold relative to a control (e.g., absence of the compound). In embodiments, the level of activity of the N-Ras protein is increased by about 5-fold relative to a control (e.g., absence of the compound). In embodiments, the level of activity of the N-Ras protein is increased by about 10-fold relative to a control (e.g., absence of the compound). In embodiments, the level of activity of the N-Ras protein is increased by about 25-fold relative to a control (e.g., absence of the compound). In embodiments, the level of activity of the N-Ras protein is increased by about 50-fold relative to a control (e.g., absence of the compound). In embodiments, the level of activity of the N-Ras protein is increased by about 100-fold relative to a control (e.g., absence of the compound). In embodiments, the level of activity of the N-Ras protein is increased by about 500-fold relative to a control (e.g., absence of the compound). In embodiments, the level of activity of the N-Ras protein is increased by about 1000-fold relative to a control (e.g., absence of the compound).

In embodiments, the level of activity of the N-Ras protein is increased by at least 1.5-fold relative to a control (e.g., absence of the compound). In embodiments, the level of activity of the N-Ras protein is increased by at least 2-fold relative to a control (e.g., absence of the compound). In embodiments, the level of activity of the N-Ras protein is increased by at least 5-fold relative to a control (e.g., absence of the compound). In embodiments, the level of activity of the N-Ras protein is increased by at least 10-fold relative to a control (e.g., absence of the compound). In embodiments, the level of activity of the N-Ras protein is increased by at least 25-fold relative to a control (e.g., absence of the compound). In embodiments, the level of activity of the N-Ras protein is increased by at least 50-fold relative to a control (e.g., absence of the compound). In embodiments, the level of activity of the N-Ras protein is increased by at least 100-fold relative to a control (e.g., absence of the compound). In embodiments, the level of activity of the N-Ras protein is increased by at least 500-fold relative to a control (e.g., absence of the compound). In embodiments, the level of activity of the N-Ras protein is increased by at least 1000-fold relative to a control (e.g., absence of the compound).

In embodiments, the modulating is reducing the activity of the N-Ras protein. In embodiments, the level of activity of the N-Ras protein is reduced by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold relative to a control (e.g., absence of the compound). In embodiments, the level of activity of the N-Ras protein is reduced by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold relative to a control (e.g., absence of the compound).

In embodiments, the level of activity of the N-Ras protein is reduced by about 1.5-fold relative to a control (e.g., absence of the compound). In embodiments, the level of activity of the N-Ras protein is reduced by about 2-fold relative to a control (e.g., absence of the compound). In embodiments, the level of activity of the N-Ras protein is reduced by about 5-fold relative to a control (e.g., absence of the compound). In embodiments, the level of activity of the N-Ras protein is reduced by about 10-fold relative to a control (e.g., absence of the compound). In embodiments, the level of activity of the N-Ras protein is reduced by about 25-fold relative to a control (e.g., absence of the compound). In embodiments, the level of activity of the N-Ras protein is reduced by about 50-fold relative to a control (e.g., absence of the compound). In embodiments, the level of activity of the N-Ras protein is reduced by about 100-fold relative to a control (e.g., absence of the compound). In embodiments, the level of activity of the N-Ras protein is reduced by about 500-fold relative to a control (e.g., absence of the compound). In embodiments, the level of activity of the N-Ras protein is reduced by about 1000-fold relative to a control (e.g., absence of the compound).

In embodiments, the level of activity of the N-Ras protein is reduced by at least 1.5-fold relative to a control (e.g., absence of the compound). In embodiments, the level of activity of the N-Ras protein is reduced by at least 2-fold relative to a control (e.g., absence of the compound). In embodiments, the level of activity of the N-Ras protein is reduced by at least 5-fold relative to a control (e.g., absence of the compound). In embodiments, the level of activity of the N-Ras protein is reduced by at least 10-fold relative to a control (e.g., absence of the compound). In embodiments, the level of activity of the N-Ras protein is reduced by at least 25-fold relative to a control (e.g., absence of the compound). In embodiments, the level of activity of the N-Ras protein is reduced by at least 50-fold relative to a control (e.g., absence of the compound). In embodiments, the level of activity of the N-Ras protein is reduced by at least 100-fold relative to a control (e.g., absence of the compound). In embodiments, the level of activity of the N-Ras protein is reduced by at least 500-fold relative to a control (e.g., absence of the compound). In embodiments, the level of activity of the N-Ras protein is reduced by at least 1000-fold relative to a control (e.g., absence of the compound).

In embodiments, the N-Ras protein is a human N-Ras protein. In embodiments, the human N-Ras protein contains a G12S mutation. In embodiments, the human N-Ras protein contains a G13S mutation. In embodiments, the human N-Ras protein contains a G12T mutation.

In embodiments, the N-Ras protein is a human N-Ras protein. In embodiments, the human N-Ras protein contains a G12D mutation. In embodiments, the human N-Ras protein contains a G13D mutation. In embodiments, the human N-Ras protein contains a G12E mutation.

### V. Protein Compositions

In an aspect is provided a Switch II GTPase protein covalently bound to a compound described herein, or a pharmaceutically acceptable salt thereof, wherein the compound is covalently bound to a serine residue of the Switch II GTPase protein.

In an aspect is provided a Switch II GTPase protein covalently bound to a compound described herein, or a pharmaceutically acceptable salt thereof, wherein the compound is covalently bound to a threonine residue of the Switch II GTPase protein.

In an aspect is provided a Switch II GTPase protein covalently bound to a compound described herein, or a pharmaceutically acceptable salt thereof, wherein the compound is covalently bound to an aspartate residue of the Switch II GTPase protein.

In an aspect is provided a Switch II GTPase protein covalently bound to a compound described herein, or a pharmaceutically acceptable salt thereof, wherein the compound is covalently bound to a glutamate residue of the Switch II GTPase protein.

In an aspect is provided a Switch II GTPase protein covalently bonded to a fragment (e.g., moiety, moiety of a fragment) of a compound described herein.

In an aspect is provided a K-Ras protein covalently bound to a compound described herein, or a pharmaceutically acceptable salt thereof, wherein the compound is covalently bound to a serine residue of the K-Ras protein. In embodiments, the K-Ras protein is a human K-Ras protein. In embodiments, the human K-Ras protein contains a G12S mutation. In embodiments, the human K-Ras protein contains a G13S mutation.

In an aspect is provided a K-Ras protein covalently bound to a compound described herein, or a pharmaceutically acceptable salt thereof, wherein the compound is covalently bound to a threonine residue of the K-Ras protein. In embodiments, the K-Ras protein is a human K-Ras protein. In embodiments, the human K-Ras protein contains a G12T mutation.

In embodiments, the compound is reversibly covalently bound to a serine residue of the K-Ras protein. In embodiments, the compound is irreversibly covalently bound to a serine residue of the K-Ras protein. In embodiments, the K-Ras protein contains a G12S mutation. In embodiments, the compound is covalently bonded to serine residue 12. In embodiments, the K-Ras protein contains a G13S mutation. In embodiments, the compound is covalently bonded to serine residue 13. In embodiments, the compound is reversibly covalently bound to a threonine residue of the K-Ras protein. In embodiments, the compound is irreversibly covalently bound to a threonine residue of the K-Ras protein. In embodiments, the K-Ras protein contains a G12T mutation. In embodiments, the compound is covalently bonded to threonine residue 12.

In an aspect is provided a K-Ras protein covalently bound to a compound described herein, or a pharmaceutically acceptable salt thereof, wherein the compound is covalently bound to an aspartate residue of the K-Ras protein. In embodiments, the K-Ras protein is a human K-Ras protein. In embodiments, the human K-Ras protein contains a G12D mutation. In embodiments, the human K-Ras protein contains a G13D mutation.

In an aspect is provided a K-Ras protein covalently bound to a compound described herein, or a pharmaceutically acceptable salt thereof, wherein the compound is covalently bound to a glutamate residue of the K-Ras protein. In embodiments, the K-Ras protein is a human K-Ras protein. In embodiments, the human K-Ras protein contains a G12E mutation.

In embodiments, the compound is reversibly covalently bound to an aspartate residue of the K-Ras protein. In embodiments, the compound is irreversibly covalently bound to an aspartate residue of the K-Ras protein. In embodiments, the K-Ras protein contains a G12D mutation. In embodiments, the compound is covalently bonded to aspartate residue 12. In embodiments, the K-Ras protein contains a G13D mutation. In embodiments, the compound is covalently bonded to aspartate residue 13. In embodiments, the compound is reversibly covalently bound to a glutamate residue of the K-Ras protein. In embodiments, the compound is irreversibly covalently bound to a glutamate residue of the K-Ras protein. In embodiments, the K-Ras protein contains a G12E mutation. In embodiments, the compound is covalently bonded to glutamate residue 12.

In embodiments, the human K-Ras protein is covalently bonded (e.g., reversibly or irreversibly) to a portion of a compound described herein.

In an aspect is provided a K-Ras protein covalently bonded to a fragment (e.g., moiety, moiety of a fragment) of a compound described herein.

In embodiments, a K-Ras(G12S) protein (e.g., human K-Ras(G12S)) is covalently bonded to a compound (e.g., compound described herein or a portion of a compound described herein). In embodiments, a K-Ras(G12S) protein (e.g., human K-Ras(G12S)) is irreversibly covalently bonded to a compound (e.g., compound described herein or a portion of a compound described herein). In embodiments, the K-Ras(G12S) protein (e.g., human K-Ras(G12S)) is reversibly covalently bonded to a compound (e.g., compound described herein or a portion of a compound described herein). In embodiments, the K-Ras(G12S) protein (e.g., human K-Ras(G12S)) is covalently bonded to a portion of a compound (e.g., compound described herein). In embodiments, the K-Ras(G12S) protein (e.g., human K-Ras(G12S)) is irreversibly covalently bonded to a portion of a compound described herein. In embodiments, the K-Ras(G12S) protein (e.g., human K-Ras(G12S)) is reversibly covalently bonded to a portion of a compound described herein. In embodiments, the compound described herein is bonded to a serine residue (e.g., G12S of human K-Ras(G12S) or serine corresponding to G12S of human K-Ras(G12S)) of the K-Ras(G12S) protein (e.g., human K-Ras(G12S)).

In embodiments, the K-Ras(G12S) protein covalently bonded to a compound described herein is the product of a reaction between the K-Ras(G12S) protein and a compound described herein. It will be understood that the covalently bonded K-Ras(G12S) protein and compound described herein are the remnants of the reactant K-Ras(G12S) protein and compound, wherein each reactant now participates in the covalent bond between the K-Ras(G12S) protein and compound. In embodiments of the covalently bonded K-Ras(G12S) protein and compound described herein, the remnant of the E substituent is a linker including a covalent bond between the K-Ras(G12S) protein and the remainder of the compound described herein. It will be understood by a person of ordinary skill in the art that when a K-Ras(G12S) protein is covalently bonded to a compound described herein, the compound described herein forms a remnant of the pre-reacted compound wherein a bond connects the remnant of the compound to the remnant of the K-Ras(G12S) protein (e.g., serine oxygen, oxygen of amino acid corresponding to G12S of human K-Ras(G12S)). In embodiments, the remnant of the E¹ substituent is a linker selected from a bond, —S(O)₂—, —NH—, —O—, —S—, —C(O)—, —C(O)NH—, —NHC(O)—, —NHC(O)NH—, —NHC(O)NH—, —C(O)O—, —OC(O)—, —CH₂NH—, substituted (e.g., substituted with a substituent group, a size-limited substituent group, or lower substituent group) or unsubstituted alkylene (e.g., C₁-C₈, C₁-C₆, C₁-C₄, or C₁-C₂), substituted (e.g., substituted with a substituent group, a size-limited substituent group, or lower substituent group) or unsubstituted heteroalkylene (e.g., 2 to 8 membered, 2 to 6 membered, 4 to 6 membered, 2 to 3 membered, or 4 to 5 membered), substituted (e.g., substituted with a substituent group, a size-limited substituent group, or lower substituent group) or unsubstituted cycloalkylene (e.g., C₃-C₈, C₃-C₆, C₄-C₆, or C₅-C₆), substituted (e.g., substituted with a substituent group, a size-limited substituent group, or lower substituent group) or unsubstituted heterocycloalkylene (e.g., 3 to 8 membered, 3 to 6 membered, 4 to 6 membered, 4 to 5 membered, or 5 to 6 membered), substituted (e.g., substituted with a substituent group, a size-limited substituent group, or lower substituent group) or unsubstituted arylene (e.g., C₆-C₁₀ or phenyl), or substituted (e.g., substituted with a substituent group, a size-limited substituent group, or lower substituent group) or unsubstituted heteroarylene (e.g., 5 to 10 membered, 5 to 9 membered, or 5 to 6 membered). As a non-limiting example, the K-Ras(G12S) protein covalently bonded to a compound may have the formula:

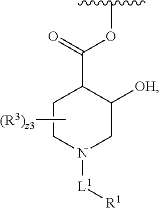

wherein O is the oxygen of a K-Ras(G12S) protein serine (e.g., corresponding to serine residue 12 of human K-Ras(G12S)), which is bonded to the remainder of the K-Ras(G12S) protein and wherein R¹, L¹, R³, and z3 are as described herein, including in embodiments.

In embodiments, a K-Ras(G13S) protein (e.g., human K-Ras(G13S)) is covalently bonded to a compound (e.g., compound described herein or a portion of a compound described herein). In embodiments, a K-Ras(G13S) protein (e.g., human K-Ras(G13S)) is irreversibly covalently bonded to a compound (e.g., compound described herein or a portion of a compound described herein). In embodiments, the K-Ras(G13S) protein (e.g., human K-Ras(G13S)) is reversibly covalently bonded to a compound (e.g., compound described herein or a portion of a compound described herein). In embodiments, the K-Ras(G13S) protein (e.g., human K-Ras(G13S)) is covalently bonded to a portion of a compound (e.g., compound described herein). In embodiments, the K-Ras(G13S) protein (e.g., human K-Ras(G13S)) is irreversibly covalently bonded to a portion of a compound described herein. In embodiments, the K-Ras(G13S) protein (e.g., human K-Ras(G13S)) is reversibly covalently bonded to a portion of a compound described herein. In embodiments, the compound described herein is bonded to a serine residue (e.g., G13S of human K-Ras(G13S) or serine corresponding to G13S of human K-Ras(G13S)) of the K-Ras(G13S) protein (e.g., human K-Ras(G13S)).

In embodiments, a K-Ras(G12T) protein (e.g., human K-Ras(G12T)) is covalently bonded to a compound (e.g., compound described herein or a portion of a compound described herein). In embodiments, a K-Ras(G12T) protein (e.g., human K-Ras(G12T)) is irreversibly covalently bonded to a compound (e.g., compound described herein or a portion of a compound described herein). In embodiments, the K-Ras(G12T) protein (e.g., human K-Ras(G12T)) is reversibly covalently bonded to a compound (e.g., compound described herein or a portion of a compound described herein). In embodiments, the K-Ras(G12T) protein (e.g., human K-Ras(G12T)) is covalently bonded to a portion of a compound (e.g., compound described herein). In embodiments, the K-Ras(G12T) protein (e.g., human K-Ras(G12T)) is irreversibly covalently bonded to a portion of a compound described herein. In embodiments, the K-Ras(G12T) protein (e.g., human K-Ras(G12T)) is reversibly covalently bonded to a portion of a compound described herein. In embodiments, the compound described herein is bonded to a threonine residue (e.g., G12T of human K-Ras(G12T) or threonine corresponding to G12T of human K-Ras(G12T)) of the K-Ras(G12T) protein (e.g., human K-Ras(G12T)).

In embodiments, a K-Ras(G12D) protein (e.g., human K-Ras(G12D)) is covalently bonded to a compound (e.g., compound described herein or a portion of a compound described herein). In embodiments, a K-Ras(G12D) protein (e.g., human K-Ras(G12D)) is irreversibly covalently bonded to a compound (e.g., compound described herein or a portion of a compound described herein). In embodiments, the K-Ras(G12D) protein (e.g., human K-Ras(G12D)) is reversibly covalently bonded to a compound (e.g., compound described herein or a portion of a compound described herein). In embodiments, the K-Ras(G12D) protein (e.g., human K-Ras(G12D)) is covalently bonded to a portion of a compound (e.g., compound described herein). In embodiments, the K-Ras(G12D) protein (e.g., human K-Ras(G12D)) is irreversibly covalently bonded to a portion of a compound described herein. In embodiments, the K-Ras(G12D) protein (e.g., human K-Ras(G12D)) is reversibly covalently bonded to a portion of a compound described herein. In embodiments, the compound described herein is bonded to an aspartate residue (e.g., G12D of human K-Ras(G12D) or aspartate corresponding to G12D of human K-Ras(G12D)) of the K-Ras(G12D) protein (e.g., human K-Ras(G12D)).

In embodiments, the K-Ras(G12D) protein covalently bonded to a compound described herein is the product of a reaction between the K-Ras(G12D) protein and a compound described herein. It will be understood that the covalently bonded K-Ras(G12D) protein and compound described herein are the remnants of the reactant K-Ras(G12D) protein and compound, wherein each reactant now participates in the covalent bond between the K-Ras(G12D) protein and compound. In embodiments of the covalently bonded K-Ras(G12D) protein and compound described herein, the remnant of the E substituent is a linker including a covalent bond between the K-Ras(G12D) protein and the remainder of the compound described herein. It will be understood by a person of ordinary skill in the art that when a K-Ras(G12D) protein is covalently bonded to a compound described herein, the compound described herein forms a remnant of the pre-reacted compound wherein a bond connects the remnant of the compound to the remnant of the K-Ras(G12D) protein (e.g., aspartate oxygen, oxygen of amino acid corresponding to G12D of human K-Ras(G12D)). In embodiments, the remnant of the E² substituent is a linker selected from a bond, —S(O)₂—, —NH—, —O—, —S—, —C(O)—, —C(O)NH—, —NHC(O)—, —NHC(O)NH—, —NHC(O)NH—, —C(O)O—, —OC(O)—, —CH₂NH—, substituted (e.g., substituted with a substituent group, a size-limited substituent group, or lower substituent group) or unsubstituted alkylene (e.g., C₁-C₈, C₁-C₆, C₁-C₄, or C₁-C₂), substituted (e.g., substituted with a substituent group, a size-limited substituent group, or lower substituent group) or unsubstituted heteroalkylene (e.g., 2 to 8 membered, 2 to 6 membered, 4 to 6 membered, 2 to 3 membered, or 4 to 5 membered), substituted (e.g., substituted with a substituent group, a size-limited substituent group, or lower substituent group) or unsubstituted cycloalkylene (e.g., C₃-C₈, C₃-C₆, C₄-C₆, or C₅-C₆), substituted (e.g., substituted with a substituent group, a size-limited substituent group, or lower substituent group) or unsubstituted heterocycloalkylene (e.g., 3 to 8 membered, 3 to 6 membered, 4 to 6 membered, 4 to 5 membered, or 5 to 6 membered), substituted (e.g., substituted with a substituent group, a size-limited substituent group, or lower substituent group) or unsubstituted arylene (e.g., C₆-C₁₀ or phenyl), or substituted (e.g., substituted with a substituent group, a size-limited substituent group, or lower substituent group) or unsubstituted heteroarylene (e.g., 5 to 10 membered, 5 to 9 membered, or 5 to 6 membered). As a non-limiting example, the K-Ras(G12D) protein covalently bonded to a compound of formula (IV-5b) may have the formula:

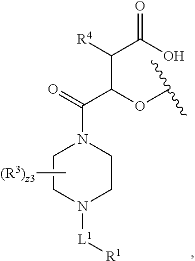

wherein the monovalent O is the oxygen of a K-Ras(G12D) protein aspartate (e.g., corresponding to aspartate residue 12 of human K-Ras(G12D)), which is bonded to the remainder of the K-Ras(G12D) protein and wherein R¹, L¹, R³, and z3 are as described herein, including in embodiments. As a further non-limiting example, the K-Ras(G12D) protein covalently bonded to a compound of formula (IV-5d) may have the formula:

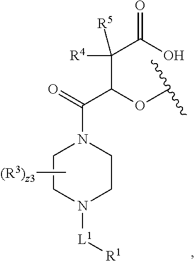

wherein the monovalent O is the oxygen of a K-Ras(G12D) protein aspartate (e.g., corresponding to aspartate residue 12 of human K-Ras(G12D)), which is bonded to the remainder of the K-Ras(G12D) protein and wherein R¹, L¹, R³, z3, R⁴, and R⁵ are as described herein, including in embodiments.

In embodiments, a K-Ras(G13D) protein (e.g., human K-Ras(G13D)) is covalently bonded to a compound (e.g., compound described herein or a portion of a compound described herein). In embodiments, a K-Ras(G13D) protein (e.g., human K-Ras(G13D)) is irreversibly covalently bonded to a compound (e.g., compound described herein or a portion of a compound described herein). In embodiments, the K-Ras(G13D) protein (e.g., human K-Ras(G13D)) is reversibly covalently bonded to a compound (e.g., compound described herein or a portion of a compound described herein). In embodiments, the K-Ras(G13D) protein (e.g., human K-Ras(G13D)) is covalently bonded to a portion of a compound (e.g., compound described herein). In embodiments, the K-Ras(G13D) protein (e.g., human K-Ras(G13D)) is irreversibly covalently bonded to a portion of a compound described herein. In embodiments, the K-Ras(G13D) protein (e.g., human K-Ras(G13D)) is reversibly covalently bonded to a portion of a compound described herein. In embodiments, the compound described herein is bonded to an aspartate residue (e.g., G13D of human K-Ras(G13D) or aspartate corresponding to G13D of human K-Ras(G13D)) of the K-Ras(G13D) protein (e.g., human K-Ras(G13D)).

In embodiments, a K-Ras(G12E) protein (e.g., human K-Ras(G12E)) is covalently bonded to a compound (e.g., compound described herein or a portion of a compound described herein). In embodiments, a K-Ras(G12E) protein (e.g., human K-Ras(G12E)) is irreversibly covalently bonded to a compound (e.g., compound described herein or a portion of a compound described herein). In embodiments, the K-Ras(G12E) protein (e.g., human K-Ras(G12E)) is reversibly covalently bonded to a compound (e.g., compound described herein or a portion of a compound described herein). In embodiments, the K-Ras(G12E) protein (e.g., human K-Ras(G12E)) is covalently bonded to a portion of a compound (e.g., compound described herein). In embodiments, the K-Ras(G12E) protein (e.g., human K-Ras(G12E)) is irreversibly covalently bonded to a portion of a compound described herein. In embodiments, the K-Ras(G12E) protein (e.g., human K-Ras(G12E)) is reversibly covalently bonded to a portion of a compound described herein. In embodiments, the compound described herein is bonded to a glutamate residue (e.g., G12E of human K-Ras(G12E) or glutamate corresponding to G12E of human K-Ras(G12E)) of the K-Ras(G12E) protein (e.g., human K-Ras(G12E)).

In embodiments, the covalently modified K-Ras protein has a modulated activity relative to a control, wherein the activity is selected from GTPase activity, nucleotide exchange, differential GDP or GTP binding, effector protein binding, effector protein activation, guanine exchange factor (GEF) binding, GEF-facilitated nucleotide exchange, phosphate release, nucleotide release, nucleotide binding, K-Ras subcellular localization, K-Ras post-translational processing, and K-Ras post-translational modifications.

In an aspect is provided an H-Ras protein covalently bound to a compound described herein, or a pharmaceutically acceptable salt thereof, wherein the compound is covalently bound to a serine residue of the H-Ras protein. In embodiments, the H-Ras protein is a human H-Ras protein. In embodiments, the human H-Ras protein contains a G12S mutation. In embodiments, the human H-Ras protein contains a G13S mutation.

In an aspect is provided an H-Ras protein covalently bound to a compound described herein, or a pharmaceutically acceptable salt thereof, wherein the compound is covalently bound to a threonine residue of the H-Ras protein. In embodiments, the H-Ras protein is a human H-Ras protein. In embodiments, the human H-Ras protein contains a G12T mutation.

In embodiments, the compound is reversibly covalently bound to a serine residue of the H-Ras protein. In embodiments, the compound is irreversibly covalently bound to a serine residue of the H-Ras protein. In embodiments, the H-Ras protein contains a G12S mutation. In embodiments, the compound is covalently bonded to serine residue 12. In embodiments, the H-Ras protein contains a G13S mutation. In embodiments, the compound is covalently bonded to serine residue 13. In embodiments, the compound is reversibly covalently bound to a threonine residue of the H-Ras protein. In embodiments, the compound is irreversibly covalently bound to a threonine residue of the H-Ras protein. In embodiments, the H-Ras protein contains a G12T mutation. In embodiments, the compound is covalently bonded to threonine residue 12.

In an aspect is provided an H-Ras protein covalently bound to a compound described herein, or a pharmaceutically acceptable salt thereof, wherein the compound is covalently bound to an aspartate residue of the H-Ras protein. In embodiments, the H-Ras protein is a human H-Ras protein. In embodiments, the human H-Ras protein contains a G12D mutation. In embodiments, the human H-Ras protein contains a G13D mutation.

In an aspect is provided an H-Ras protein covalently bound to a compound described herein, or a pharmaceutically acceptable salt thereof, wherein the compound is covalently bound to a glutamate residue of the H-Ras protein. In embodiments, the H-Ras protein is a human H-Ras protein. In embodiments, the human H-Ras protein contains a G12E mutation.

In embodiments, the compound is reversibly covalently bound to an aspartate residue of the H-Ras protein. In embodiments, the compound is irreversibly covalently bound to an aspartate residue of the H-Ras protein. In embodiments, the H-Ras protein contains a G12D mutation. In embodiments, the compound is covalently bonded to aspartate residue 12. In embodiments, the H-Ras protein contains a G13D mutation. In embodiments, the compound is covalently bonded to aspartate residue 13. In embodiments, the compound is reversibly covalently bound to a glutate residue of the H-Ras protein. In embodiments, the compound is irreversibly covalently bound to a glutamate residue of the H-Ras protein. In embodiments, the H-Ras protein contains a G12E mutation. In embodiments, the compound is covalently bonded to glutamate residue 12.

In embodiments, the human H-Ras protein is covalently bonded (e.g., reversibly or irreversibly) to a portion of a compound described herein.

In an aspect is provided an H-Ras protein covalently bonded to a fragment (e.g., moiety, moiety of a fragment) of a compound described herein.

In embodiments, an H-Ras(G12S) protein (e.g., human H-Ras(G12S)) is covalently bonded to a compound (e.g., compound described herein or a portion of a compound described herein). In embodiments, an H-Ras(G12S) protein (e.g., human H-Ras(G12S)) is irreversibly covalently bonded to a compound (e.g., compound described herein or a portion of a compound described herein). In embodiments, the H-Ras(G12S) protein (e.g., human H-Ras(G12S)) is reversibly covalently bonded to a compound (e.g., compound described herein or a portion of a compound described herein). In embodiments, the H-Ras(G12S) protein (e.g., human H-Ras(G12S)) is covalently bonded to a portion of a compound (e.g., compound described herein). In embodiments, the H-Ras(G12S) protein (e.g., human H-Ras(G12S)) is irreversibly covalently bonded to a portion of a compound described herein. In embodiments, the H-Ras(G12S) protein (e.g., human H-Ras(G12S)) is reversibly covalently bonded to a portion of a compound described herein. In embodiments, the compound described herein is bonded to a serine residue (e.g., G12S of human H-Ras(G12S) or serine corresponding to G12S of human H-Ras(G12S)) of the H-Ras(G12S) protein (e.g., human H-Ras(G12S)).

In embodiments, the H-Ras(G12S) protein covalently bonded to a compound described herein is the product of a reaction between the H-Ras(G12S) protein and a compound described herein. It will be understood that the covalently bonded H-Ras(G12S) protein and compound described herein are the remnants of the reactant H-Ras(G12S) protein and compound, wherein each reactant now participates in the covalent bond between the H-Ras(G12S) protein and compound. In embodiments of the covalently bonded H-Ras(G12S) protein and compound described herein, the remnant of the E substituent is a linker including a covalent bond between the H-Ras(G12S) protein and the remainder of the compound described herein. It will be understood by a person of ordinary skill in the art that when an H-Ras(G12S) protein is covalently bonded to a compound described herein, the compound described herein forms a remnant of the pre-reacted compound wherein a bond connects the remnant of the compound to the remnant of the H-Ras(G12S) protein (e.g., serine oxygen, oxygen of amino acid corresponding to G12S of human H-Ras(G12S)). In embodiments, the remnant of the E¹ substituent is a linker selected from a bond, —S(O)₂—, —NH—, —O—, —S—, —C(O)—, —C(O)NH—, —NHC(O)—, —NHC(O)NH—, —NHC(O)NH—, —C(O)O—, —OC(O)—, —CH₂NH—, substituted (e.g., substituted with a substituent group, a size-limited substituent group, or lower substituent group) or unsubstituted alkylene (e.g., C₁-C₈, C₁-C₆, C₁-C₄, or C₁-C₂), substituted (e.g., substituted with a substituent group, a size-limited substituent group, or lower substituent group) or unsubstituted heteroalkylene (e.g., 2 to 8 membered, 2 to 6 membered, 4 to 6 membered, 2 to 3 membered, or 4 to 5 membered), substituted (e.g., substituted with a substituent group, a size-limited substituent group, or lower substituent group) or unsubstituted cycloalkylene (e.g., C₃-C₈, C₃-C₆, C₄-C₆, or C₅-C₆), substituted (e.g., substituted with a substituent group, a size-limited substituent group, or lower substituent group) or unsubstituted heterocycloalkylene (e.g., 3 to 8 membered, 3 to 6 membered, 4 to 6 membered, 4 to 5 membered, or 5 to 6 membered), substituted (e.g., substituted with a substituent group, a size-limited substituent group, or lower substituent group) or unsubstituted arylene (e.g., C₆-C₁₀ or phenyl), or substituted (e.g., substituted with a substituent group, a size-limited substituent group, or lower substituent group) or unsubstituted heteroarylene (e.g., 5 to 10 membered, 5 to 9 membered, or 5 to 6 membered). As a non-limiting example, the H-Ras(G12S) protein covalently bonded to a compound may have the formula:

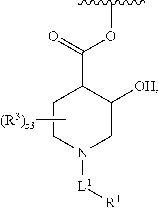

wherein O is the oxygen of an H-Ras(G12S) protein serine (e.g., corresponding to serine residue 12 of human H-Ras(G12S)), which is bonded to the remainder of the H-Ras(G12S) protein and wherein R¹, L¹, R³, and z3 are as described herein, including in embodiments.

In embodiments, an H-Ras(G13S) protein (e.g., human H-Ras(G13S)) is covalently bonded to a compound (e.g., compound described herein or a portion of a compound described herein). In embodiments, an H-Ras(G13S) protein (e.g., human H-Ras(G13S)) is irreversibly covalently bonded to a compound (e.g., compound described herein or a portion of a compound described herein). In embodiments, the H-Ras(G13S) protein (e.g., human H-Ras(G13S)) is reversibly covalently bonded to a compound (e.g., compound described herein or a portion of a compound described herein). In embodiments, the H-Ras(G13S) protein (e.g., human H-Ras(G13S)) is covalently bonded to a portion of a compound (e.g., compound described herein). In embodiments, the H-Ras(G13S) protein (e.g., human H-Ras(G13S)) is irreversibly covalently bonded to a portion of a compound described herein. In embodiments, the H-Ras(G13S) protein (e.g., human H-Ras(G13S)) is reversibly covalently bonded to a portion of a compound described herein. In embodiments, the compound described herein is bonded to a serine residue (e.g., G13S of human H-Ras(G13S) or serine corresponding to G13S of human H-Ras(G13S)) of the H-Ras(G13S) protein (e.g., human H-Ras(G13S)).

In embodiments, an H-Ras(G12T) protein (e.g., human H-Ras(G12T)) is covalently bonded to a compound (e.g., compound described herein or a portion of a compound described herein). In embodiments, an H-Ras(G12T) protein (e.g., human H-Ras(G12T)) is irreversibly covalently bonded to a compound (e.g., compound described herein or a portion of a compound described herein). In embodiments, the H-Ras(G12T) protein (e.g., human H-Ras(G12T)) is reversibly covalently bonded to a compound (e.g., compound described herein or a portion of a compound described herein). In embodiments, the H-Ras(G12T) protein (e.g., human H-Ras(G12T)) is covalently bonded to a portion of a compound (e.g., compound described herein). In embodiments, the H-Ras(G12T) protein (e.g., human H-Ras(G12T)) is irreversibly covalently bonded to a portion of a compound described herein. In embodiments, the H-Ras(G12T) protein (e.g., human H-Ras(G12T)) is reversibly covalently bonded to a portion of a compound described herein. In embodiments, the compound described herein is bonded to a threonine residue (e.g., G12T of human H-Ras(G12T) or threonine corresponding to G12T of human H-Ras(G12T)) of the H-Ras(G12T) protein (e.g., human H-Ras(G12T)).

In embodiments, an H-Ras(G12D) protein (e.g., human H-Ras(G12D)) is covalently bonded to a compound (e.g., compound described herein or a portion of a compound described herein). In embodiments, an H-Ras(G12D) protein (e.g., human H-Ras(G12D)) is irreversibly covalently bonded to a compound (e.g., compound described herein or a portion of a compound described herein). In embodiments, the H-Ras(G12D) protein (e.g., human H-Ras(G12D)) is reversibly covalently bonded to a compound (e.g., compound described herein or a portion of a compound described herein). In embodiments, the H-Ras(G12D) protein (e.g., human H-Ras(G12D)) is covalently bonded to a portion of a compound (e.g., compound described herein). In embodiments, the H-Ras(G12D) protein (e.g., human H-Ras(G12D)) is irreversibly covalently bonded to a portion of a compound described herein. In embodiments, the H-Ras(G12D) protein (e.g., human H-Ras(G12D)) is reversibly covalently bonded to a portion of a compound described herein. In embodiments, the compound described herein is bonded to an aspartate residue (e.g., G12D of human H-Ras(G12D) or aspartate corresponding to G12D of human H-Ras(G12D)) of the H-Ras(G12D) protein (e.g., human H-Ras(G12D)).

In embodiments, the H-Ras(G12D) protein covalently bonded to a compound described herein is the product of a reaction between the H-Ras(G12D) protein and a compound described herein. It will be understood that the covalently bonded H-Ras(G12D) protein and compound described herein are the remnants of the reactant H-Ras(G12D) protein and compound, wherein each reactant now participates in the covalent bond between the H-Ras(G12D) protein and compound. In embodiments of the covalently bonded H-Ras(G12D) protein and compound described herein, the remnant of the E substituent is a linker including a covalent bond between the H-Ras(G12D) protein and the remainder of the compound described herein. It will be understood by a person of ordinary skill in the art that when an H-Ras(G12D) protein is covalently bonded to a compound described herein, the compound described herein forms a remnant of the pre-reacted compound wherein a bond connects the remnant of the compound to the remnant of the H-Ras(G12D) protein (e.g., aspartate oxygen, oxygen of amino acid corresponding to G12D of human H-Ras(G12D)). In embodiments, the remnant of the E² substituent is a linker selected from a bond, —S(O)₂—, —NH—, —O—, —S—, —C(O)—, —C(O)NH—, —NHC(O)—, —NHC(O)NH—, —NHC(O)NH—, —C(O)O—, —OC(O)—, —CH₂NH—, substituted (e.g., substituted with a substituent group, a size-limited substituent group, or lower substituent group) or unsubstituted alkylene (e.g., C₁-C₈, C₁-C₆, C₁-C₄, or C₁-C₂), substituted (e.g., substituted with a substituent group, a size-limited substituent group, or lower substituent group) or unsubstituted heteroalkylene (e.g., 2 to 8 membered, 2 to 6 membered, 4 to 6 membered, 2 to 3 membered, or 4 to 5 membered), substituted (e.g., substituted with a substituent group, a size-limited substituent group, or lower substituent group) or unsubstituted cycloalkylene (e.g., C₃-C₈, C₃-C₆, C₄-C₆, or C₅-C₆), substituted (e.g., substituted with a substituent group, a size-limited substituent group, or lower substituent group) or unsubstituted heterocycloalkylene (e.g., 3 to 8 membered, 3 to 6 membered, 4 to 6 membered, 4 to 5 membered, or 5 to 6 membered), substituted (e.g., substituted with a substituent group, a size-limited substituent group, or lower substituent group) or unsubstituted arylene (e.g., C₆-C₁₀ or phenyl), or substituted (e.g., substituted with a substituent group, a size-limited substituent group, or lower substituent group) or unsubstituted heteroarylene (e.g., 5 to 10 membered, 5 to 9 membered, or 5 to 6 membered). As a non-limiting example, the H-Ras(G12D) protein covalently bonded to a compound of formula (IV-5b) may have the formula:

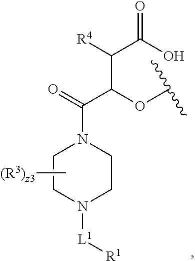

wherein the monovalent O is the oxygen of an H-Ras(G12D) protein aspartate (e.g., corresponding to aspartate residue 12 of human H-Ras(G12D)), which is bonded to the remainder of the H-Ras(G12D) protein and wherein R¹, L¹, R³, and z3 are as described herein, including in embodiments. As a further non-limiting example, the H-Ras(G12D) protein covalently bonded to a compound of formula (IV-5d) may have the formula:

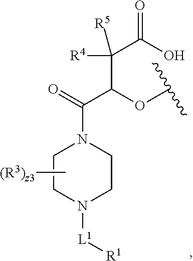

wherein the monovalent O is the oxygen of an H-Ras(G12D) protein aspartate (e.g., corresponding to aspartate residue 12 of human H-Ras(G12D)), which is bonded to the remainder of the H-Ras(G12D) protein and wherein R¹, L¹, R³, z3, R⁴, and R⁵ are as described herein, including in embodiments.

In embodiments, an H-Ras(G13D) protein (e.g., human H-Ras(G13D)) is covalently bonded to a compound (e.g., compound described herein or a portion of a compound described herein). In embodiments, an H-Ras(G13D) protein (e.g., human H-Ras(G13D)) is irreversibly covalently bonded to a compound (e.g., compound described herein or a portion of a compound described herein). In embodiments, the H-Ras(G13D) protein (e.g., human H-Ras(G13D)) is reversibly covalently bonded to a compound (e.g., compound described herein or a portion of a compound described herein). In embodiments, the H-Ras(G13D) protein (e.g., human H-Ras(G13D)) is covalently bonded to a portion of a compound (e.g., compound described herein). In embodiments, the H-Ras(G13D) protein (e.g., human H-Ras(G13D)) is irreversibly covalently bonded to a portion of a compound described herein. In embodiments, the H-Ras(G13D) protein (e.g., human H-Ras(G13D)) is reversibly covalently bonded to a portion of a compound described herein. In embodiments, the compound described herein is bonded to an aspartate residue (e.g., G13D of human H-Ras(G13D) or aspartate corresponding to G13D of human H-Ras(G13D)) of the H-Ras(G13D) protein (e.g., human H-Ras(G13D)).

In embodiments, an H-Ras(G12E) protein (e.g., human H-Ras(G12E)) is covalently bonded to a compound (e.g., compound described herein or a portion of a compound described herein). In embodiments, an H-Ras(G12E) protein (e.g., human H-Ras(G12E)) is irreversibly covalently bonded to a compound (e.g., compound described herein or a portion of a compound described herein). In embodiments, the H-Ras(G12E) protein (e.g., human H-Ras(G12E)) is reversibly covalently bonded to a compound (e.g., compound described herein or a portion of a compound described herein). In embodiments, the H-Ras(G12E) protein (e.g., human H-Ras(G12E)) is covalently bonded to a portion of a compound (e.g., compound described herein). In embodiments, the H-Ras(G12E) protein (e.g., human H-Ras(G12E)) is irreversibly covalently bonded to a portion of a compound described herein. In embodiments, the H-Ras(G12E) protein (e.g., human H-Ras(G12E)) is reversibly covalently bonded to a portion of a compound described herein. In embodiments, the compound described herein is bonded to a glutamate residue (e.g., G12E of human H-Ras(G12E) or glutamate corresponding to G12E of human H-Ras(G12E)) of the H-Ras(G12E) protein (e.g., human H-Ras(G12E)).

In embodiments, the covalently modified H-Ras protein has a modulated activity relative to a control, wherein the activity is selected from GTPase activity, nucleotide exchange, differential GDP or GTP binding, effector protein binding, effector protein activation, guanine exchange factor (GEF) binding, GEF-facilitated nucleotide exchange, phosphate release, nucleotide release, nucleotide binding, H-Ras subcellular localization, H-Ras post-translational processing, and H-Ras post-translational modifications.

In an aspect is provided an N-Ras protein covalently bound to a compound described herein, or a pharmaceutically acceptable salt thereof, wherein the compound is covalently bound to a serine residue of the N-Ras protein. In embodiments, the N-Ras protein is a human N-Ras protein. In embodiments, the human N-Ras protein contains a G12S mutation. In embodiments, the human N-Ras protein contains a G13S mutation.

In an aspect is provided an N-Ras protein covalently bound to a compound described herein, or a pharmaceutically acceptable salt thereof, wherein the compound is covalently bound to a threonine residue of the N-Ras protein. In embodiments, the N-Ras protein is a human N-Ras protein. In embodiments, the human N-Ras protein contains a G12T mutation.

In embodiments, the compound is reversibly covalently bound to a serine residue of the N-Ras protein. In embodiments, the compound is irreversibly covalently bound to a serine residue of the N-Ras protein. In embodiments, the N-Ras protein contains a G12S mutation. In embodiments, the compound is covalently bonded to serine residue 12. In embodiments, the N-Ras protein contains a G13S mutation. In embodiments, the compound is covalently bonded to serine residue 13. In embodiments, the compound is reversibly covalently bound to a threonine residue of the N-Ras protein. In embodiments, the compound is irreversibly covalently bound to a threonine residue of the N-Ras protein. In embodiments, the N-Ras protein contains a G12T mutation. In embodiments, the compound is covalently bonded to threonine residue 12.

In an aspect is provided an N-Ras protein covalently bound to a compound described herein, or a pharmaceutically acceptable salt thereof, wherein the compound is covalently bound to an aspartate residue of the N-Ras protein. In embodiments, the N-Ras protein is a human N-Ras protein. In embodiments, the human N-Ras protein contains a G12D mutation. In embodiments, the human N-Ras protein contains a G13D mutation.

In an aspect is provided an N-Ras protein covalently bound to a compound described herein, or a pharmaceutically acceptable salt thereof, wherein the compound is covalently bound to a glutamate residue of the N-Ras protein. In embodiments, the N-Ras protein is a human N-Ras protein. In embodiments, the human N-Ras protein contains a G12E mutation.

In embodiments, the compound is reversibly covalently bound to an aspartate residue of the N-Ras protein. In embodiments, the compound is irreversibly covalently bound to an aspartate residue of the N-Ras protein. In embodiments, the N-Ras protein contains a G12D mutation. In embodiments, the compound is covalently bonded to aspartate residue 12. In embodiments, the N-Ras protein contains a G13D mutation. In embodiments, the compound is covalently bonded to aspartate residue 13. In embodiments, the compound is reversibly covalently bound to a glutamate residue of the N-Ras protein. In embodiments, the compound is irreversibly covalently bound to a glutamate residue of the N-Ras protein. In embodiments, the N-Ras protein contains a G12E mutation. In embodiments, the compound is covalently bonded to glutamate residue 12.

In embodiments, the human N-Ras protein is covalently bonded (e.g., reversibly or irreversibly) to a portion of a compound described herein.

In an aspect is provided an N-Ras protein covalently bonded to a fragment (e.g., moiety, moiety of a fragment) of a compound described herein.

In embodiments, an N-Ras(G12S) protein (e.g., human N-Ras(G12S)) is covalently bonded to a compound (e.g., compound described herein or a portion of a compound described herein). In embodiments, an N-Ras(G12S) protein (e.g., human N-Ras(G12S)) is irreversibly covalently bonded to a compound (e.g., compound described herein or a portion of a compound described herein). In embodiments, the N-Ras(G12S) protein (e.g., human N-Ras(G12S)) is reversibly covalently bonded to a compound (e.g., compound described herein or a portion of a compound described herein). In embodiments, the N-Ras(G12S) protein (e.g., human N-Ras(G12S)) is covalently bonded to a portion of a compound (e.g., compound described herein). In embodiments, the N-Ras(G12S) protein (e.g., human N-Ras(G12S)) is irreversibly covalently bonded to a portion of a compound described herein. In embodiments, the N-Ras(G12S) protein (e.g., human N-Ras(G12S)) is reversibly covalently bonded to a portion of a compound described herein. In embodiments, the compound described herein is bonded to a serine residue (e.g., G12S of human N-Ras(G12S) or serine corresponding to G12S of human N-Ras(G12S)) of the N-Ras(G12S) protein (e.g., human N-Ras(G12S)).

In embodiments, the N-Ras(G12S) protein covalently bonded to a compound described herein is the product of a reaction between the N-Ras(G12S) protein and a compound described herein. It will be understood that the covalently bonded N-Ras(G12S) protein and compound described herein are the remnants of the reactant N-Ras(G12S) protein and compound, wherein each reactant now participates in the covalent bond between the N-Ras(G12S) protein and compound. In embodiments of the covalently bonded N-Ras(G12S) protein and compound described herein, the remnant of the E substituent is a linker including a covalent bond between the N-Ras(G12S) protein and the remainder of the compound described herein. It will be understood by a person of ordinary skill in the art that when an N-Ras(G12S) protein is covalently bonded to a compound described herein, the compound described herein forms a remnant of the pre-reacted compound wherein a bond connects the remnant of the compound to the remnant of the N-Ras(G12S) protein (e.g., serine oxygen, oxygen of amino acid corresponding to G12S of human N-Ras(G12S)). In embodiments, the remnant of the E¹ substituent is a linker selected from a bond, —S(O)₂—, —NH—, —O—, —S—, —C(O)—, —C(O)NH—, —NHC(O)—, —NHC(O)NH—, —NHC(O)NH—, —C(O)O—, —OC(O)—, —CH₂NH—, substituted (e.g., substituted with a substituent group, a size-limited substituent group, or lower substituent group) or unsubstituted alkylene (e.g., C₁-C₈, C₁-C₆, C₁-C₄, or C₁-C₂), substituted (e.g., substituted with a substituent group, a size-limited substituent group, or lower substituent group) or unsubstituted heteroalkylene (e.g., 2 to 8 membered, 2 to 6 membered, 4 to 6 membered, 2 to 3 membered, or 4 to 5 membered), substituted (e.g., substituted with a substituent group, a size-limited substituent group, or lower substituent group) or unsubstituted cycloalkylene (e.g., C₃-C₈, C₃-C₆, C₄-C₆, or C₅-C₆), substituted (e.g., substituted with a substituent group, a size-limited substituent group, or lower substituent group) or unsubstituted heterocycloalkylene (e.g., 3 to 8 membered, 3 to 6 membered, 4 to 6 membered, 4 to 5 membered, or 5 to 6 membered), substituted (e.g., substituted with a substituent group, a size-limited substituent group, or lower substituent group) or unsubstituted arylene (e.g., C₆-C₁₀ or phenyl), or substituted (e.g., substituted with a substituent group, a size-limited substituent group, or lower substituent group) or unsubstituted heteroarylene (e.g., 5 to 10 membered, 5 to 9 membered, or 5 to 6 membered). As a non-limiting example, the N-Ras(G12S) protein covalently bonded to a compound may have the formula:

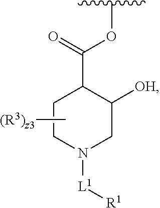

wherein O is the oxygen of an N-Ras(G12S) protein serine (e.g., corresponding to serine residue 12 of human N-Ras(G12S)), which is bonded to the remainder of the N-Ras(G12S) protein and wherein R¹, L¹, R³, and z3 are as described herein, including in embodiments.

In embodiments, an N-Ras(G13S) protein (e.g., human N-Ras(G13S)) is covalently bonded to a compound (e.g., compound described herein or a portion of a compound described herein). In embodiments, an N-Ras(G13S) protein (e.g., human N-Ras(G13S)) is irreversibly covalently bonded to a compound (e.g., compound described herein or a portion of a compound described herein). In embodiments, the N-Ras(G13S) protein (e.g., human N-Ras(G13S)) is reversibly covalently bonded to a compound (e.g., compound described herein or a portion of a compound described herein). In embodiments, the N-Ras(G13S) protein (e.g., human N-Ras(G13S)) is covalently bonded to a portion of a compound (e.g., compound described herein). In embodiments, the N-Ras(G13S) protein (e.g., human N-Ras(G13S)) is irreversibly covalently bonded to a portion of a compound described herein. In embodiments, the N-Ras(G13S) protein (e.g., human N-Ras(G13S)) is reversibly covalently bonded to a portion of a compound described herein. In embodiments, the compound described herein is bonded to a serine residue (e.g., G13S of human N-Ras(G13S) or serine corresponding to G13S of human N-Ras(G13S)) of the N-Ras(G13S) protein (e.g., human N-Ras(G13S)).

In embodiments, an N-Ras(G12T) protein (e.g., human N-Ras(G12T)) is covalently bonded to a compound (e.g., compound described herein or a portion of a compound described herein). In embodiments, an N-Ras(G12T) protein (e.g., human N-Ras(G12T)) is irreversibly covalently bonded to a compound (e.g., compound described herein or a portion of a compound described herein). In embodiments, the N-Ras(G12T) protein (e.g., human N-Ras(G12T)) is reversibly covalently bonded to a compound (e.g., compound described herein or a portion of a compound described herein). In embodiments, the N-Ras(G12T) protein (e.g., human N-Ras(G12T)) is covalently bonded to a portion of a compound (e.g., compound described herein). In embodiments, the N-Ras(G12T) protein (e.g., human N-Ras(G12T)) is irreversibly covalently bonded to a portion of a compound described herein. In embodiments, the N-Ras(G12T) protein (e.g., human N-Ras(G12T)) is reversibly covalently bonded to a portion of a compound described herein. In embodiments, the compound described herein is bonded to a threonine residue (e.g., G12T of human N-Ras(G12T) or threonine corresponding to G12T of human N-Ras(G12T)) of the N-Ras(G12T) protein (e.g., human N-Ras(G12T)).

In embodiments, an N-Ras(G12D) protein (e.g., human N-Ras(G12D)) is covalently bonded to a compound (e.g., compound described herein or a portion of a compound described herein). In embodiments, an N-Ras(G12D) protein (e.g., human N-Ras(G12D)) is irreversibly covalently bonded to a compound (e.g., compound described herein or a portion of a compound described herein). In embodiments, the N-Ras(G12D) protein (e.g., human N-Ras(G12D)) is reversibly covalently bonded to a compound (e.g., compound described herein or a portion of a compound described herein). In embodiments, the N-Ras(G12D) protein (e.g., human N-Ras(G12D)) is covalently bonded to a portion of a compound (e.g., compound described herein). In embodiments, the N-Ras(G12D) protein (e.g., human N-Ras(G12D)) is irreversibly covalently bonded to a portion of a compound described herein. In embodiments, the N-Ras(G12D) protein (e.g., human N-Ras(G12D)) is reversibly covalently bonded to a portion of a compound described herein. In embodiments, the compound described herein is bonded to an aspartate residue (e.g., G12D of human N-Ras(G12D) or aspartate corresponding to G12D of human N-Ras(G12D)) of the N-Ras(G12D) protein (e.g., human N-Ras(G12D)).

In embodiments, the N-Ras(G12D) protein covalently bonded to a compound described herein is the product of a reaction between the N-Ras(G12D) protein and a compound described herein. It will be understood that the covalently bonded N-Ras(G12D) protein and compound described herein are the remnants of the reactant N-Ras(G12D) protein and compound, wherein each reactant now participates in the covalent bond between the N-Ras(G12D) protein and compound. In embodiments of the covalently bonded N-Ras(G12D) protein and compound described herein, the remnant of the E substituent is a linker including a covalent bond between the N-Ras(G12D) protein and the remainder of the compound described herein. It will be understood by a person of ordinary skill in the art that when an N-Ras(G12D) protein is covalently bonded to a compound described herein, the compound described herein forms a remnant of the pre-reacted compound wherein a bond connects the remnant of the compound to the remnant of the N-Ras(G12D) protein (e.g., aspartate oxygen, oxygen of amino acid corresponding to G12D of human N-Ras(G12D)). In embodiments, the remnant of the E² substituent is a linker selected from a bond, —S(O)₂—, —NH—, —O—, —S—, —C(O)—, —C(O)NH—, —NHC(O)—, —NHC(O)NH—, —NHC(O)NH—, —C(O)O—, —OC(O)—, —CH₂NH—, substituted (e.g., substituted with a substituent group, a size-limited substituent group, or lower substituent group) or unsubstituted alkylene (e.g., C₁-C₈, C₁-C₆, C₁-C₄, or C₁-C₂), substituted (e.g., substituted with a substituent group, a size-limited substituent group, or lower substituent group) or unsubstituted heteroalkylene (e.g., 2 to 8 membered, 2 to 6 membered, 4 to 6 membered, 2 to 3 membered, or 4 to 5 membered), substituted (e.g., substituted with a substituent group, a size-limited substituent group, or lower substituent group) or unsubstituted cycloalkylene (e.g., C₃-C₈, C₃-C₆, C₄-C₆, or C₅-C₆), substituted (e.g., substituted with a substituent group, a size-limited substituent group, or lower substituent group) or unsubstituted heterocycloalkylene (e.g., 3 to 8 membered, 3 to 6 membered, 4 to 6 membered, 4 to 5 membered, or 5 to 6 membered), substituted (e.g., substituted with a substituent group, a size-limited substituent group, or lower substituent group) or unsubstituted arylene (e.g., C₆-C₁₀ or phenyl), or substituted (e.g., substituted with a substituent group, a size-limited substituent group, or lower substituent group) or unsubstituted heteroarylene (e.g., 5 to 10 membered, 5 to 9 membered, or 5 to 6 membered). As a non-limiting example, the N-Ras(G12D) protein covalently bonded to a compound of formula (IV-5d) may have the formula:

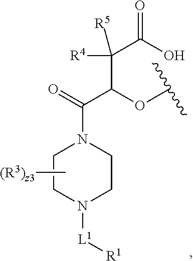

wherein the monovalent O is the oxygen of an N-Ras(G12D) protein aspartate (e.g., corresponding to aspartate residue 12 of human N-Ras(G12D)), which is bonded to the remainder of the N-Ras(G12D) protein and wherein R¹, L¹, R³, z3, R⁴, and R⁵ are as described herein, including in embodiments.

In embodiments, an N-Ras(G13D) protein (e.g., human N-Ras(G13D)) is covalently bonded to a compound (e.g., compound described herein or a portion of a compound described herein). In embodiments, an N-Ras(G13D) protein (e.g., human N-Ras(G13D)) is irreversibly covalently bonded to a compound (e.g., compound described herein or a portion of a compound described herein). In embodiments, the N-Ras(G13D) protein (e.g., human N-Ras(G13D)) is reversibly covalently bonded to a compound (e.g., compound described herein or a portion of a compound described herein). In embodiments, the N-Ras(G13D) protein (e.g., human N-Ras(G13D)) is covalently bonded to a portion of a compound (e.g., compound described herein). In embodiments, the N-Ras(G13D) protein (e.g., human N-Ras(G13D)) is irreversibly covalently bonded to a portion of a compound described herein. In embodiments, the N-Ras(G13D) protein (e.g., human N-Ras(G13D)) is reversibly covalently bonded to a portion of a compound described herein. In embodiments, the compound described herein is bonded to an aspartate residue (e.g., G13D of human N-Ras(G13D) or aspartate corresponding to G13D of human N-Ras(G13D)) of the N-Ras(G13D) protein (e.g., human N-Ras(G13D)).

In embodiments, an N-Ras(G12E) protein (e.g., human N-Ras(G12E)) is covalently bonded to a compound (e.g., compound described herein or a portion of a compound described herein). In embodiments, an N-Ras(G12E) protein (e.g., human N-Ras(G12E)) is irreversibly covalently bonded to a compound (e.g., compound described herein or a portion of a compound described herein). In embodiments, the N-Ras(G12E) protein (e.g., human N-Ras(G12E)) is reversibly covalently bonded to a compound (e.g., compound described herein or a portion of a compound described herein). In embodiments, the N-Ras(G12E) protein (e.g., human N-Ras(G12E)) is covalently bonded to a portion of a compound (e.g., compound described herein). In embodiments, the N-Ras(G12E) protein (e.g., human N-Ras(G12E)) is irreversibly covalently bonded to a portion of a compound described herein. In embodiments, the N-Ras(G12E) protein (e.g., human N-Ras(G12E)) is reversibly covalently bonded to a portion of a compound described herein. In embodiments, the compound described herein is bonded to a glutamate residue (e.g., G12E of human N-Ras(G12E) or glutamate corresponding to G12E of human N-Ras(G12E)) of the N-Ras(G12E) protein (e.g., human N-Ras(G12E)).

In embodiments, the covalently modified N-Ras protein has a modulated activity relative to a control, wherein the activity is selected from GTPase activity, nucleotide exchange, differential GDP or GTP binding, effector protein binding, effector protein activation, guanine exchange factor (GEF) binding, GEF-facilitated nucleotide exchange, phosphate release, nucleotide release, nucleotide binding, N-Ras subcellular localization, N-Ras post-translational processing, and N-Ras post-translational modifications.

### VI. Embodiments

Embodiment Q1. A compound, or a pharmaceutically acceptable salt thereof, having the formula:

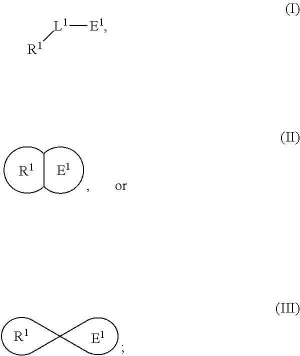

wherein
R¹ is a Switch II Binding Pocket binding moiety;
L¹ is a bond or divalent linker; and
E¹ is an electrophilic moiety capable of forming a covalent bond with a Switch II GTPase protein serine residue or a Switch II GTPase protein threonine residue.

Embodiment Q2. The compound of embodiment Q1, wherein E¹ is an electrophilic moiety capable of forming a covalent bond with a Switch II GTPase protein serine residue.

Embodiment Q3. The compound of embodiment Q2, wherein the Switch II GTPase protein serine residue is a natural Switch II GTPase protein serine residue.

Embodiment Q4. The compound of embodiment Q2, wherein the Switch II GTPase protein serine residue is a mutant Switch II GTPase protein serine residue.

Embodiment Q5. The compound of embodiment Q4, wherein the mutant Switch II GTPase protein serine residue is serine residue 12 of K-Ras(G12S), H-Ras(G12S), or N-Ras(G12S).

Embodiment Q6. The compound of embodiment Q4, wherein the mutant Switch II GTPase protein serine residue is serine residue 13 of K-Ras(G13S), H-Ras(G13S), or N-Ras(G13S).

Embodiment Q7. The compound of embodiment Q1, wherein E¹ is an electrophilic moiety capable of forming a covalent bond with a Switch II GTPase protein threonine residue.

Embodiment Q8. The compound of embodiment Q7, wherein the Switch II GTPase protein threonine residue is a natural Switch II GTPase protein threonine residue.

Embodiment Q9. The compound of embodiment Q7, wherein the Switch II GTPase protein threonine residue is a mutant Switch II GTPase protein serine threonine.

Embodiment Q10. The compound of embodiment Q9, wherein the mutant Switch II GTPase protein threonine residue is threonine residue 12 of K-Ras(G12T), H-Ras(G12T), or N-Ras(G12T).

Embodiment Q11. The compound of one of embodiments Q1 to Q10, wherein E¹ comprises a β-lactone.

Embodiment Q12. The compound of one of embodiments Q1 to Q10, wherein E¹ comprises a β-lactam.

Embodiment Q13. The compound of one of embodiments Q1 to Q12, having the formula:

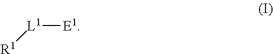

Embodiment Q14. The compound of embodiment Q13, having the formula:

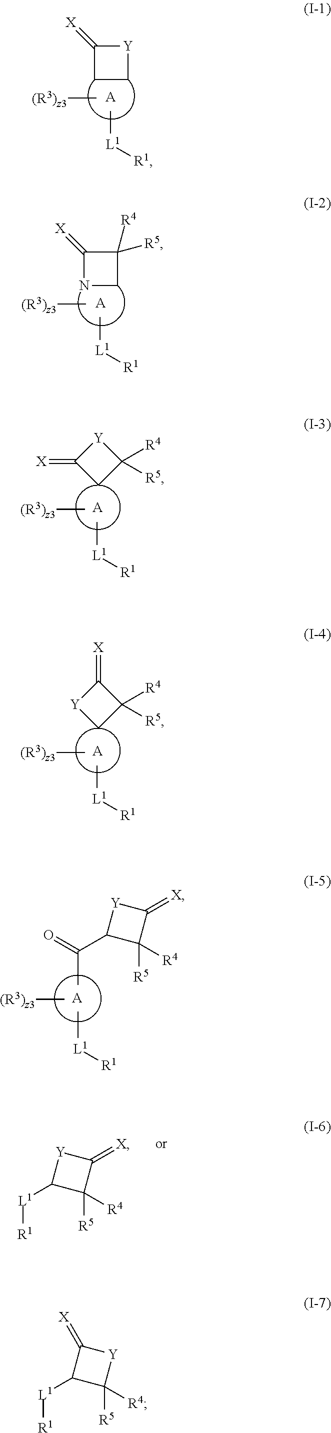

wherein
Ring A is a cycloalkyl or heterocycloalkyl;
X is O or S;
Y is O, S, or NR²;
R² is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³ is independently oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R³ substituents may optionally be joined to form a substituted or unsubstituted cycloalkyl, substituted or unsubstituted heterocycloalkyl, substituted or unsubstituted aryl, or substituted or unsubstituted heteroaryl;
z3 is an integer from 0 to 10;
R⁴ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and
R⁵ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment Q15. The compound of embodiment Q14, wherein Ring A is a 5 to 6 membered heterocycloalkyl.

Embodiment Q16. The compound of embodiment Q14, wherein Ring A is a piperidinyl, pyrrolidinyl, or piperazinyl.

Embodiment Q17. The compound of embodiment Q14, having the formula:

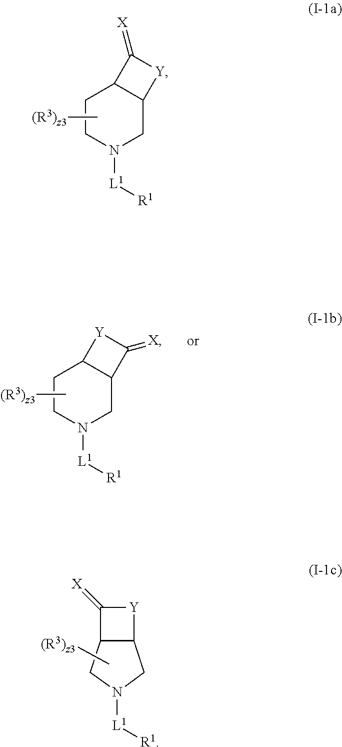

Embodiment Q18. The compound of embodiment Q17, having the formula:

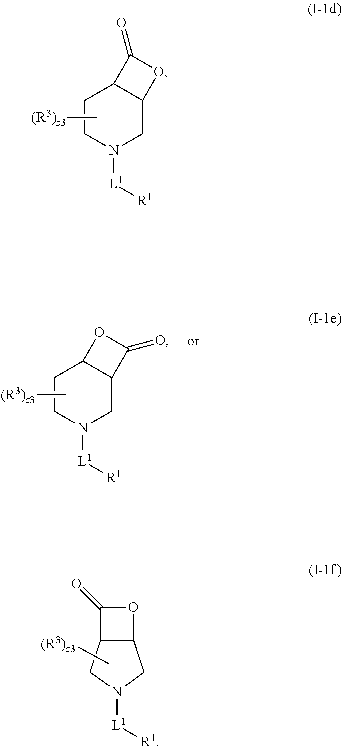

Embodiment Q19. The compound of embodiment Q14, having the formula:

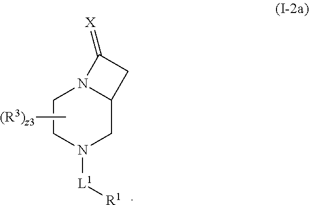

Embodiment Q20. The compound of embodiment Q19, having the formula:

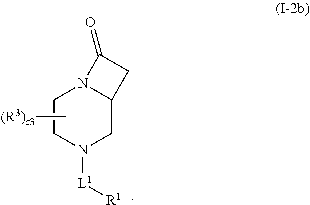

Embodiment Q21. The compound of embodiment Q14, having the formula:

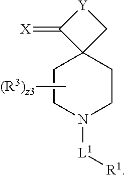

Embodiment Q22. The compound of embodiment Q21, having the formula:

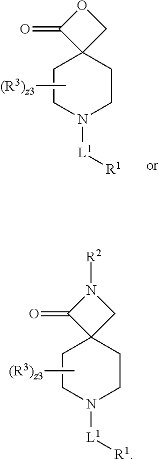

Embodiment Q23. The compound of embodiment Q14, having the formula:

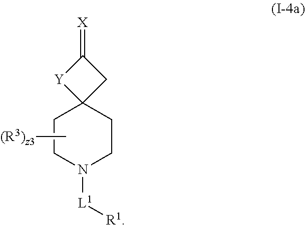

Embodiment Q24. The compound of embodiment Q23, having the formula:

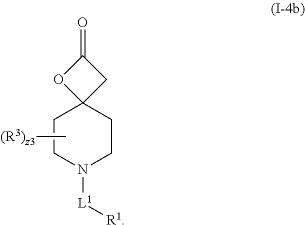

Embodiment Q25. The compound of embodiment Q14, having the formula:

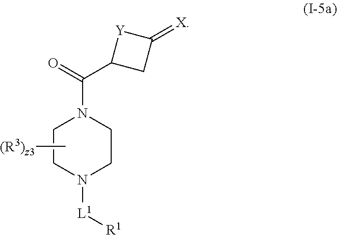

Embodiment Q26. The compound of embodiment Q25, having the formula:

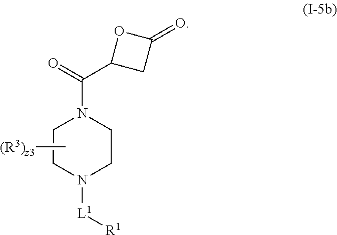

Embodiment Q27. The compound of embodiment Q14, having the formula:

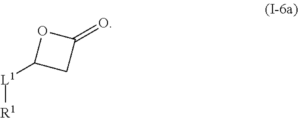

Embodiment Q28. The compound of embodiment Q14, having the formula:

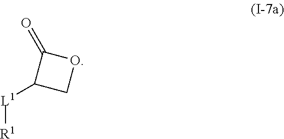

Embodiment Q29. The compound of one of embodiments Q14 to Q26, wherein R³ is independently unsubstituted C₁-C₄ alkyl.

Embodiment Q30. The compound of one of embodiments Q14 to Q26, wherein R³ is independently unsubstituted methyl.

Embodiment Q31. The compound of one of embodiments Q14 to Q26, wherein two R³ substituents are joined to form a substituted or unsubstituted cycloalkyl or substituted or unsubstituted heterocycloalkyl.

Embodiment Q32. The compound of one of embodiments Q1 to Q31, wherein
L¹ is -L¹⁰¹-L¹⁰²-L¹⁰³-;
L¹⁰¹ is connected directly to E¹;
L¹⁰¹ is a bond, —C(O)—, —C(O)O—, —OC(O)—, —O—, —S—, —NR¹⁰¹—, —C(O)NR¹⁰¹—, —NR¹⁰¹C(O)—, —NR¹⁰¹C(O)O—, —OC(O)NR¹⁰¹—, —NR¹⁰¹C(O)NR¹⁰¹—, —NR¹⁰¹C(NH)NR¹⁰¹—, —S(O)₂—, —NR¹⁰¹S(O)₂—, —S(O)₂NR¹⁰¹—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L¹⁰² is a bond, —C(O)—, —C(O)O—, —OC(O)—, —O—, —S—, —NR¹⁰²—, —C(O)NR¹⁰²—, —NR¹⁰²C(O)—, —NR¹⁰²C(O)O—, —OC(O)NR¹⁰²—, —NR¹⁰²C(O)NR¹⁰²—, —NR¹⁰²C(NH)NR¹⁰²—, —S(O)₂—, —NR¹⁰²S(O)₂—, —S(O)₂NR¹⁰²—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L¹⁰³ is a bond, —C(O)—, —C(O)O—, —OC(O)—, —O—, —S—, —NR¹⁰³—, —C(O)NR¹⁰³—, —NR¹⁰³C(O)—, —NR¹⁰³C(O)O—, —OC(O)NR¹⁰³—, —NR¹⁰³C(O)NR¹⁰³—, —NR¹⁰³C(NH)NR¹⁰³—, —S(O)₂—, —NR¹⁰³S(O)₂—, —S(O)₂NR¹⁰³—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; and
each R¹⁰¹, R¹⁰², and R¹⁰³ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment Q33. The compound of one of embodiments Q1 to Q31, wherein L¹ is a bond.

Embodiment Q34. The compound of one of embodiments Q1 to Q31, wherein L¹ is —C(O)—.

Embodiment Q35. The compound of one of embodiments Q1 to Q31, wherein L¹ is a substituted 2 to 6 membered heteroalkylene.

Embodiment Q36. The compound of one of embodiments Q1 to Q31, wherein L¹ is

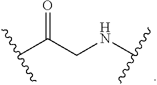 [SMILES: *NCC(*)=O]

Embodiment Q37. The compound of one of embodiments Q1 to Q36, wherein R¹ is -L²⁰-R²⁰.
L²⁰ is a bond, —C(O)—, —C(O)O—, —OC(O)—, —O—, —S—, —NR²⁰⁰—, —C(O)NR²⁰⁰—, —NR²⁰⁰C(O)—, —NR²⁰⁰C(O)O—, —OC(O)NR²⁰⁰—, —NR²⁰⁰C(O)NR²⁰⁰—, —NR²⁰⁰C(NH)NR²⁰⁰—, —S(O)₂—, —NR²⁰⁰S(O)₂—, —S(O)₂NR²⁰⁰—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R²⁰⁰ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R²⁰ is hydrogen, halogen, —CX²⁰₃, —CHX²⁰₂, —CH₂X²⁰, —OCX²⁰₃, —OCH₂X²⁰, —OCHX²⁰₂, —CN, —SOₙ₂₀R^{20D}, —SOᵥ₂₀NR^{20A}R^{20B}, —NR^{20C}NR^{20A}R^{20B}, —ONR^{20A}R^{20B}, —NHC(O)NR^{20C}NR^{20A}R²⁰, —NHC(O)NR^{20A}R²⁰, —N(O)ₘ₂₀, —NR^{20A}R^{20B}, —C(O)R^{20C}, —C(O)OR^{20C}, —C(O)NR^{20A}R^{20B}, —OR^{20D}, —SR^{20D}, —NR^{20A}SO₂R^{20D}, —NR^{20A}C(O)R^{20C}, —NR^{20A}C(O)OR^{20C}, —NR^{20A}OR^{20C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{20A}, R^{20B}, R^{20C}, and R^{20D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{20A} and R^{20B} substituents bonded to the same nitrogen atom may optionally be joined to form a substituted or unsubstituted heterocycloalkyl or substituted or unsubstituted heteroaryl;
X²⁰ is independently —F, —Cl, —Br, or —I;
n20 is an integer from 0 to 4; and
m20 and v20 are independently 1 or 2.

Embodiment Q38. The compound of one of embodiments Q1 to Q36, wherein R¹ is

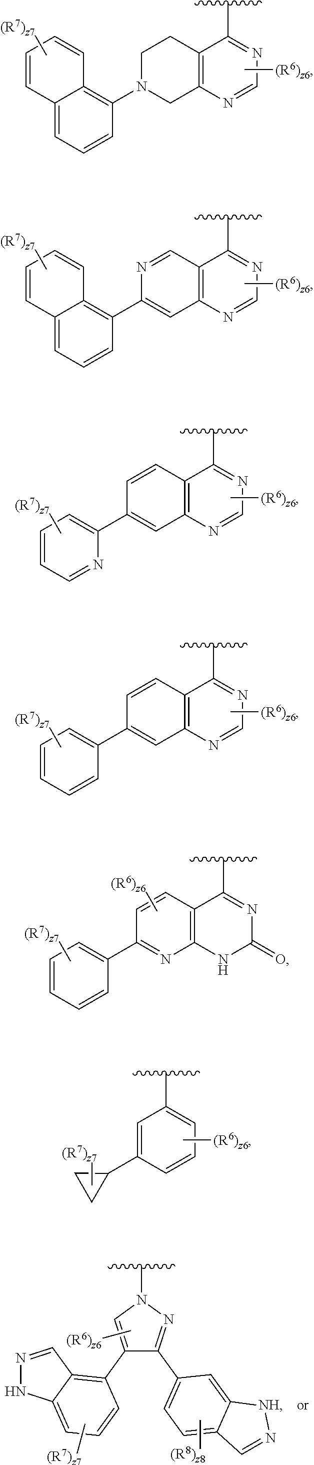

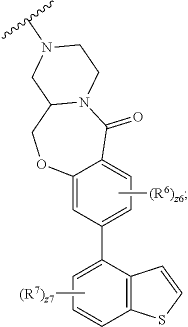

R⁶ is independently oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁷ is independently oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁸ is independently halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
z6 is an integer from 0 to 7;
z7 is an integer from 0 to 7; and
z8 is an integer from 0 to 5.

Embodiment Q39. The compound of embodiment Q38, wherein R⁶ is independently a halogen, —OH, unsubstituted C₁-C₄ alkyl, substituted 2 to 6 membered heteroalkyl, or substituted 5 to 6 membered heteroaryl.

Embodiment Q40. The compound of embodiment Q38, wherein R⁶ is independently —F, —Cl, —OH, or unsubstituted methyl.

Embodiment Q41. The compound of embodiment Q38, wherein R⁶ is independently a 2 to 6 membered heteroalkyl, substituted with substituted heterocycloalkyl or unsubstituted fused heterocycloalkyl.

Embodiment Q42. The compound of embodiment Q38, wherein R⁶ is independently a substituted pyridyl.

Embodiment Q43. The compound of one of embodiments Q38 to Q42, wherein z6 is 1, 2, or 3.

Embodiment Q44. The compound of one of embodiments Q38 to Q43, wherein R⁷ is independently a halogen, —CF₃, —CN, —OH, —NH₂, or unsubstituted C₁-C₄ alkyl.

Embodiment Q45. The compound of one of embodiments Q38 to Q43, wherein R⁷ is independently —F, —Cl, —CF₃, —CN, —OH, —NH₂, or unsubstituted methyl.

Embodiment Q46. The compound of one of embodiments Q38 to Q45, wherein z7 is 1, 2, or 3.

Embodiment Q47. The compound of one of embodiments Q38 to Q46, wherein R⁸ is independently a halogen or unsubstituted C₁-C₄ alkyl.

Embodiment Q48. The compound of one of embodiments Q38 to Q46, wherein R⁸ is independently —Cl or unsubstituted methyl.

Embodiment Q49. The compound of one of embodiments Q38 to Q48, wherein z8 is 1.

Embodiment Q50. The compound of one of embodiments Q1 to Q36, wherein R¹ is

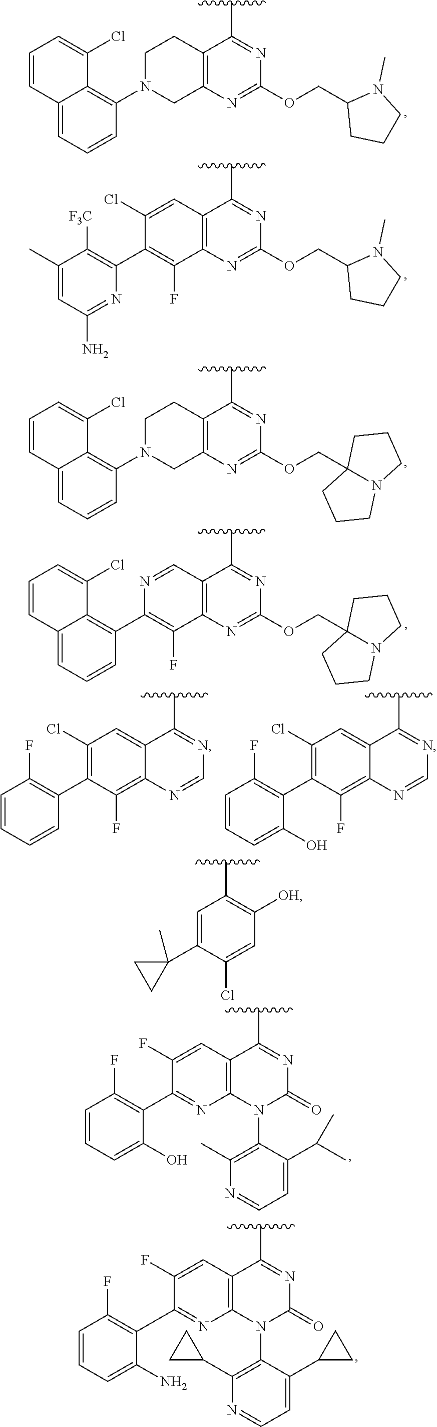

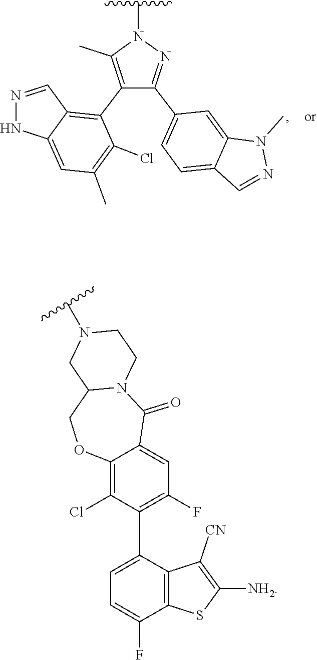 [SMILES: *n1nc(-c2ccc3cnn(C)c3c2)c(-c2c(Cl)c(C)cc3[nH]ncc23)c1C ; *N1CCN2C(=O)c3cc(F)c(-c4ccc(F)c5sc(N)c(C#N)c45)c(Cl)c3OCC2C1]

Embodiment Q51. The compound of one of embodiments Q1 to Q12, having the formula:

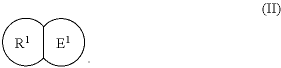

Embodiment Q52. The compound of embodiment Q51, having the formula:

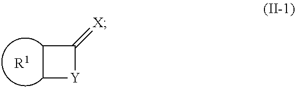

wherein
X is O or S;
Y is O, S, or NR²; and
R² is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment Q53. The compound of embodiment Q52, having the formula:

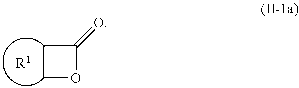

Embodiment Q54. The compound of one of embodiments Q51 to Q53, wherein R¹ is

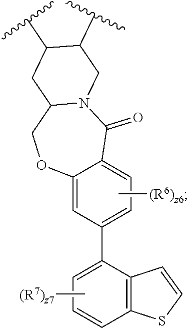

wherein
R⁶ is independently oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁷ is independently halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
z6 is an integer from 0 to 3; and
z7 is an integer from 0 to 5.

Embodiment Q55. The compound of embodiment Q54, wherein R¹ is

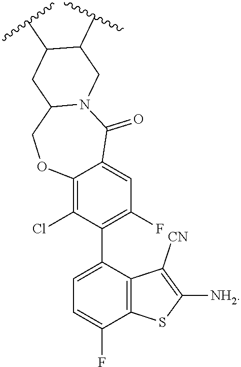 [SMILES: *C1CC2COc3c(cc(F)c(-c4ccc(F)c5sc(N)c(C#N)c45)c3Cl)C(=O)N2CC1*]

Embodiment Q56. The compound of one of embodiments Q1 to Q12, having the formula:

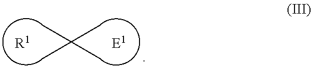

Embodiment Q57. The compound of embodiment Q56, having the formula:

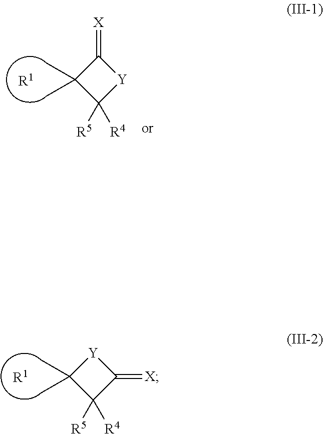

wherein
X is O or S;
Y is O, S, or NR²; R² is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁴ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and
R⁵ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment Q58. The compound of embodiment Q57, having the formula:

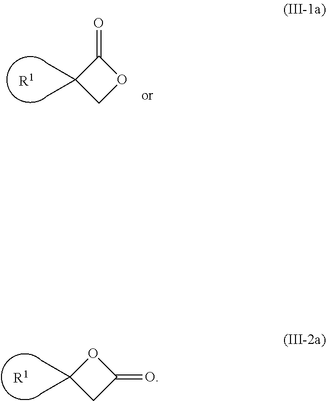

Embodiment Q59. The compound of one of embodiments Q56 to Q58, wherein R¹ is

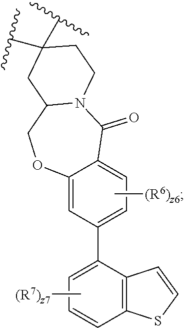

wherein
R⁶ is independently oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁷ is independently halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
z6 is an integer from 0 to 3; and
z7 is an integer from 0 to 5.

Embodiment Q60. The compound of embodiment Q59, wherein R¹ is

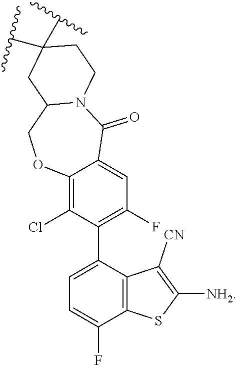 [SMILES: *C1(*)CCN2C(=O)c3cc(F)c(-c4ccc(F)c5sc(N)c(C#N)c45)c(Cl)c3OCC2C1]

Embodiment Q61. A pharmaceutical composition comprising a pharmaceutically acceptable excipient and a compound of one of embodiments Q1 to Q60, or a pharmaceutically acceptable salt thereof.

Embodiment Q62. A method of treating cancer in a subject in need thereof, said method comprising administering to the subject in need thereof a therapeutically effective amount of a compound of one of embodiments Q1 to Q60, or a pharmaceutically acceptable salt thereof.

Embodiment Q63. The method of embodiment Q62, wherein the cancer is rectal carcinoma, colorectal adenocarcinoma, colorectal carcinoma, non-small cell lung carcinoma, squamous cell lung carcinoma, myelodysplastic syndrome, or acute myeloid leukemia.

Embodiment Q64. A method of treating a K-Ras(G12S)-associated disease in a subject in need thereof, said method comprising administering to the subject in need thereof a therapeutically effective amount of a compound of one of embodiments Q1 to Q60, or a pharmaceutically acceptable salt thereof.

Embodiment Q65. A method of treating an H-Ras(G12S)-associated disease in a subject in need thereof, said method comprising administering to the subject in need thereof a therapeutically effective amount of a compound of one of embodiments Q1 to Q60, or a pharmaceutically acceptable salt thereof.

Embodiment Q66. The method of embodiment Q65, wherein said H-Ras(G12S)-associated disease is Costello syndrome.

Embodiment Q67. A method of treating an N-Ras(G12S)-associated disease in a subject in need thereof, said method comprising administering to the subject in need thereof a therapeutically effective amount of a compound of one of embodiments Q1 to Q60, or a pharmaceutically acceptable salt thereof.

Embodiment Q68. A method of modulating the level of activity of a K-Ras protein in a cell, said method comprising contacting the cell with an effective amount of a compound of one of embodiments Q1 to Q60, or a pharmaceutically acceptable salt thereof.

Embodiment Q69. The method of embodiment Q68, wherein said modulating of said activity comprises modulating GTPase activity, nucleotide exchange, differential GDP or GTP binding, effector protein binding, effector protein activation, guanine exchange factor (GEF) binding, GEF-facilitated nucleotide exchange, phosphate release, nucleotide release, nucleotide binding, K-Ras subcellular localization, K-Ras post-translational processing, or K-Ras post-translational modifications.

Embodiment Q70. The method of one of embodiments Q68 to Q69, wherein said modulating is increasing the activity of said K-Ras protein.

Embodiment Q71. The method of one of embodiments Q68 to Q69, wherein said modulating is reducing the activity of said K-Ras protein.

Embodiment Q72. The method of one of embodiments Q68 to Q71, wherein said K-Ras protein is a human K-Ras protein.

Embodiment Q73. The method of embodiment Q72, wherein said human K-Ras protein contains a G12S mutation.

Embodiment Q74. A method of modulating the level of activity of an H-Ras protein in a cell, said method comprising contacting the cell with an effective amount of a compound of one of embodiments Q1 to Q60, or a pharmaceutically acceptable salt thereof.

Embodiment Q75. The method of embodiment Q74, wherein said modulating of said activity comprises modulating GTPase activity, nucleotide exchange, differential GDP or GTP binding, effector protein binding, effector protein activation, guanine exchange factor (GEF) binding, GEF-facilitated nucleotide exchange, phosphate release, nucleotide release, nucleotide binding, H-Ras subcellular localization, H-Ras post-translational processing, or H-Ras post-translational modifications.

Embodiment Q76. The method of one of embodiments Q74 to Q75, wherein said modulating is increasing the activity of said H-Ras protein.

Embodiment Q77. The method of one of embodiments Q74 to Q75, wherein said modulating is reducing the activity of said H-Ras protein.

Embodiment Q78. The method of one of embodiments Q74 to Q77, wherein said H-Ras protein is a human H-Ras protein.

Embodiment Q79. The method of embodiment Q78, wherein said human H-Ras protein contains a G12S mutation.

Embodiment Q80. A method of modulating the level of activity of an N-Ras protein in a cell, said method comprising contacting the cell with an effective amount of a compound of one of embodiments Q1 to Q60, or a pharmaceutically acceptable salt thereof.

Embodiment Q81. The method of embodiment Q80, wherein said modulating of said activity comprises modulating GTPase activity, nucleotide exchange, differential GDP or GTP binding, effector protein binding, effector protein activation, guanine exchange factor (GEF) binding, GEF-facilitated nucleotide exchange, phosphate release, nucleotide release, nucleotide binding, N-Ras subcellular localization, N-Ras post-translational processing, or N-Ras post-translational modifications.

Embodiment Q82. The method of one of embodiments Q80 to Q81, wherein said modulating is increasing the activity of said N-Ras protein.

Embodiment Q83. The method of one of embodiments Q80 to Q81, wherein said modulating is reducing the activity of said N-Ras protein.

Embodiment Q84. The method of one of embodiments Q80 to Q83, wherein said N-Ras protein is a human K-Ras protein.

Embodiment Q85. The method of embodiment Q84, wherein said human N-Ras protein contains a G12S mutation.

Embodiment Q86. A K-Ras protein covalently bound to a compound of one of embodiments Q1 to Q60, or a pharmaceutically acceptable salt thereof, wherein said compound is covalently bound to a serine residue of said K-Ras protein.

Embodiment Q87. The covalently modified K-Ras protein of embodiment Q86, wherein said compound is reversibly covalently bound to a serine residue of said K-Ras protein.

Embodiment Q88. The covalently modified K-Ras protein of embodiment Q86, wherein said compound is irreversibly covalently bound to a serine residue of said K-Ras protein.

Embodiment Q89. The covalently modified K-Ras protein of one of embodiments Q86 to Q88, wherein said covalently modified K-Ras protein has a modulated activity relative to a control, wherein said activity is selected from GTPase activity, nucleotide exchange, differential GDP or GTP binding, effector protein binding, effector protein activation, guanine exchange factor (GEF) binding, GEF-facilitated nucleotide exchange, phosphate release, nucleotide release, nucleotide binding, K-Ras subcellular localization, K-Ras post-translational processing, and K-Ras post-translational modifications.

Embodiment Q90. The covalently modified K-Ras protein of one of embodiments Q86 to Q89, wherein said K-Ras protein contains a G12S mutation.

Embodiment Q91. The covalently modified K-Ras protein of embodiment Q90, wherein said compound is covalently bonded to serine residue 12.

Embodiment Q92. An H-Ras protein covalently bound to a compound of one of embodiments Q1 to Q60, or a pharmaceutically acceptable salt thereof, wherein said compound is covalently bound to a serine residue of said H-Ras protein.

Embodiment Q93. The covalently modified H-Ras protein of embodiment Q92, wherein said compound is reversibly covalently bound to a serine residue of said H-Ras protein.

Embodiment Q94. The covalently modified H-Ras protein of embodiment Q92, wherein said compound is irreversibly covalently bound to a serine residue of said H-Ras protein.

Embodiment Q95. The covalently modified H-Ras protein of one of embodiments Q92 to Q94, wherein said covalently modified H-Ras protein has a modulated activity relative to a control, wherein said activity is selected from GTPase activity, nucleotide exchange, differential GDP or GTP binding, effector protein binding, effector protein activation, guanine exchange factor (GEF) binding, GEF-facilitated nucleotide exchange, phosphate release, nucleotide release, nucleotide binding, H-Ras subcellular localization, H-Ras post-translational processing, and H-Ras post-translational modifications.

Embodiment Q96. The covalently modified H-Ras protein of one of embodiments Q92 to Q95, wherein said H-Ras protein contains a G12S mutation.

Embodiment Q97. The covalently modified H-Ras protein of embodiment Q96, wherein said compound is covalently bonded to serine residue 12.

Embodiment Q98. An N-Ras protein covalently bound to a compound of one of embodiments Q1 to Q60, or a pharmaceutically acceptable salt thereof, wherein said compound is covalently bound to a serine residue of said N-Ras protein.

Embodiment Q99. The covalently modified N-Ras protein of embodiment Q98, wherein said compound is reversibly covalently bound to a serine residue of said N-Ras protein.

Embodiment Q100. The covalently modified N-Ras protein of embodiment Q98, wherein said compound is irreversibly covalently bound to a serine residue of said N-Ras protein.

Embodiment Q101. The covalently modified N-Ras protein of one of embodiments Q98 to Q100, wherein said covalently modified H-Ras protein has a modulated activity relative to a control, wherein said activity is selected from GTPase activity, nucleotide exchange, differential GDP or GTP binding, effector protein binding, effector protein activation, guanine exchange factor (GEF) binding, GEF-facilitated nucleotide exchange, phosphate release, nucleotide release, nucleotide binding, N-Ras subcellular localization, N-Ras post-translational processing, and N-Ras post-translational modifications.

Embodiment Q102. The covalently modified N-Ras protein of one of embodiments Q98 to Q101, wherein said H-Ras protein contains a G12S mutation.

Embodiment Q103. The covalently modified N-Ras protein of embodiment Q102, wherein said compound is covalently bonded to serine residue 12.

Embodiment D1. A compound, or a pharmaceutically acceptable salt thereof, having the formula:

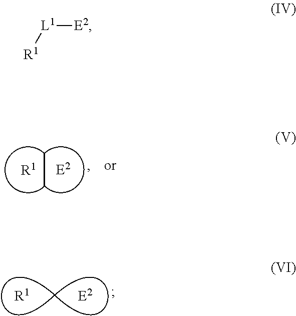

wherein
R¹ is a Switch II Binding Pocket binding moiety;
L¹ is a bond or divalent linker; and
E² is an electrophilic moiety capable of forming a covalent bond with a Switch II GTPase protein aspartate residue or a Switch II GTPase protein glutamate residue.

Embodiment D2. The compound of embodiment D1, wherein E² is an electrophilic moiety capable of forming a covalent bond with a Switch II GTPase protein aspartate residue.

Embodiment D3. The compound of embodiment D2, wherein the Switch II GTPase protein aspartate residue is a natural Switch II GTPase protein aspartate residue.

Embodiment D4. The compound of embodiment D2, wherein the Switch II GTPase protein aspartate residue is a mutant Switch II GTPase protein aspartate residue.

Embodiment D5. The compound of embodiment D4, wherein the mutant Switch II GTPase protein aspartate residue is aspartate residue 12 of K-Ras(G12D), H-Ras(G12D), or N-Ras(G12D).

Embodiment D6. The compound of embodiment D4, wherein the mutant Switch II GTPase protein aspartate residue is aspartate residue 13 of K-Ras(G13D), H-Ras(G13D), or N-Ras(G13D).

Embodiment D7. The compound of embodiment D1, wherein E² is an electrophilic moiety capable of forming a covalent bond with a Switch II GTPase protein glutamate residue.

Embodiment D8. The compound of embodiment D7, wherein the Switch II GTPase protein glutamate residue is a natural Switch II GTPase protein glutamate residue.

Embodiment D9. The compound of embodiment D7, wherein the Switch II GTPase protein glutamate residue is a mutant Switch II GTPase protein glutamate residue.

Embodiment D10. The compound of one of embodiments D1 to D9, wherein E² comprises a β-lactone.

Embodiment D11. The compound of one of embodiments D1 to D9, wherein E² comprises a β-lactam.

Embodiment D12. The compound of one of embodiments D1 to D11, having the formula:

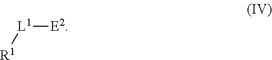

Embodiment D13. The compound of embodiment D12, having the formula:

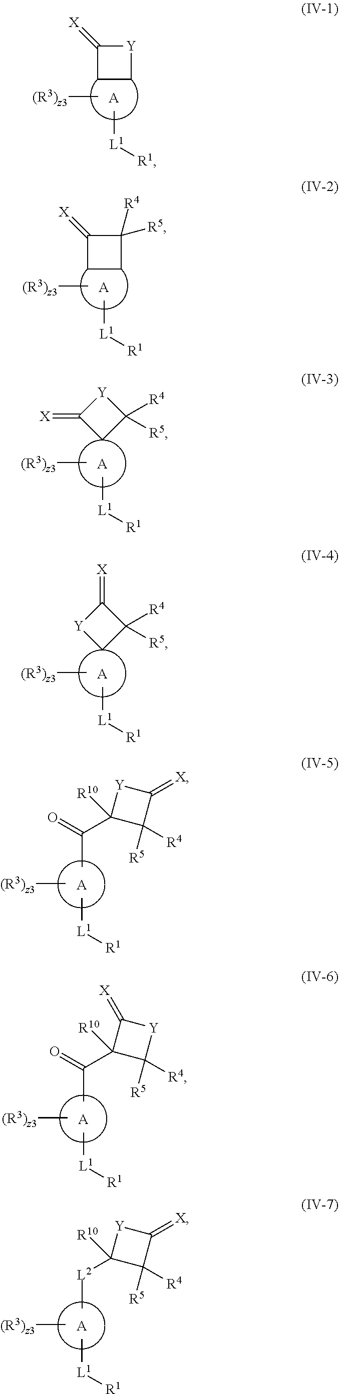

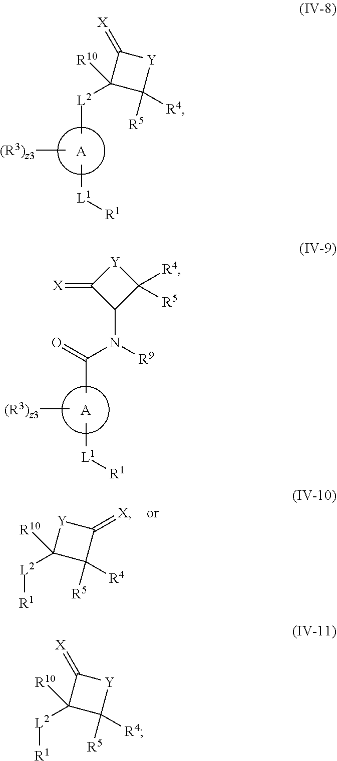

wherein
Ring A is a cycloalkyl or heterocycloalkyl;
L² is unsubstituted C₁-C₄ alkylene;
X is O or S;
Y is O, S, or NR²;
R² is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³ is independently oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R³ substituents may optionally be joined to form a substituted or unsubstituted cycloalkyl, substituted or unsubstituted heterocycloalkyl, substituted or unsubstituted aryl, or substituted or unsubstituted heteroaryl;
z3 is an integer from 0 to 10;
R⁴ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁵ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁹ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and
R¹⁰ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment D14. The compound of embodiment D13, wherein Ring A is a 5 to 6 membered heterocycloalkyl.

Embodiment D15. The compound of embodiment D13, wherein Ring A is a piperidinyl, pyrrolidinyl, or piperazinyl.

Embodiment D16. The compound of embodiment D13, having the formula:

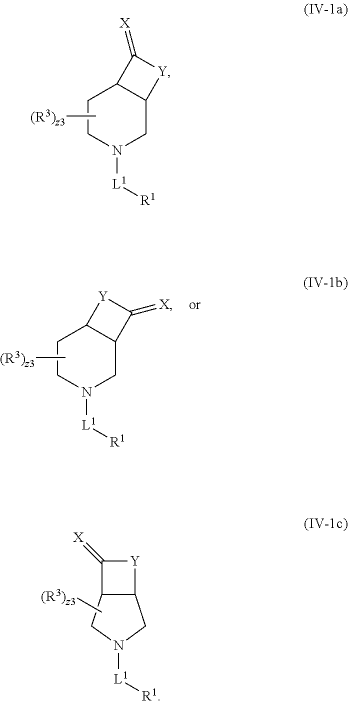

Embodiment D17. The compound of embodiment D16, having the formula:

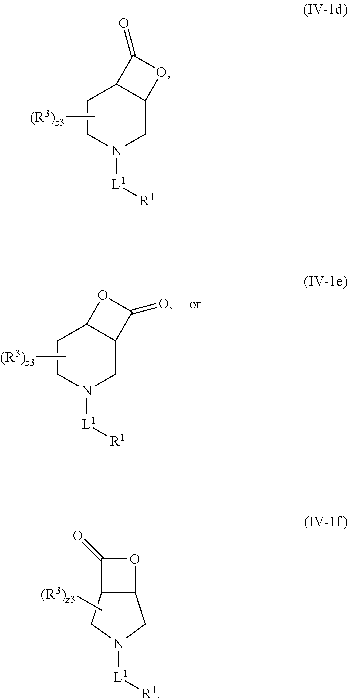

Embodiment D18. The compound of embodiment D13, having the formula:

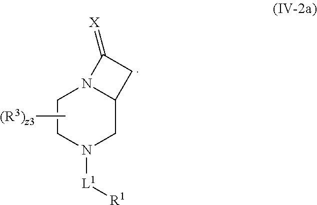

Embodiment D19. The compound of embodiment D18, having the formula:

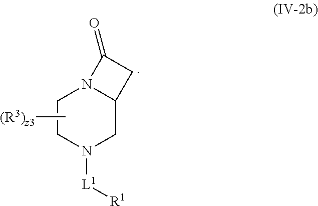

Embodiment D20. The compound of embodiment D13, having the formula:

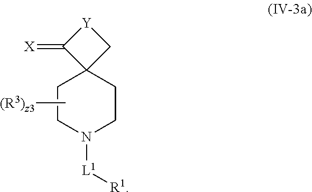

Embodiment D21. The compound of embodiment D20, having the formula:

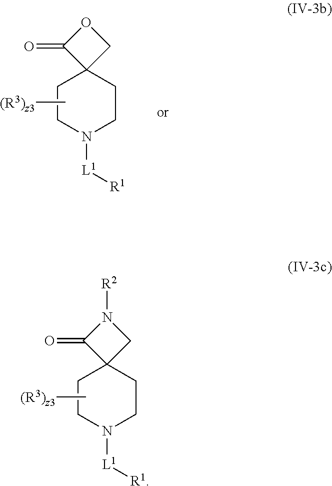

Embodiment D22. The compound of embodiment D13, having the formula:

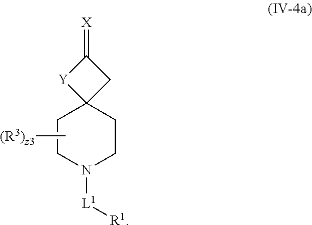

Embodiment D23. The compound of embodiment D22, having the formula:

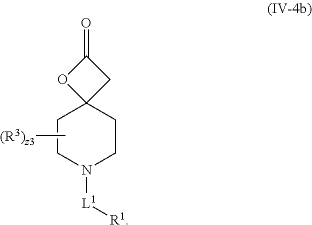

Embodiment D24. The compound of embodiment D13, having the formula:

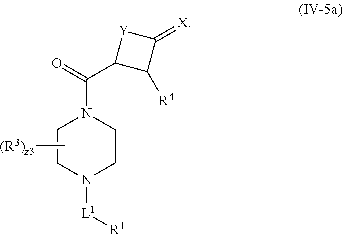

Embodiment D25. The compound of embodiment D24, having the formula:

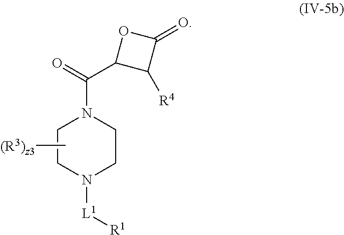

Embodiment D26. The compound of embodiment D13, having the formula:

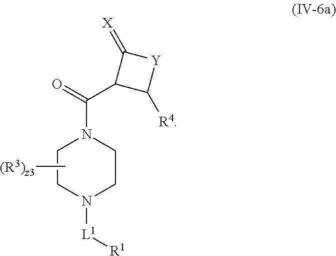

Embodiment D27. The compound of embodiment D26, having the formula:

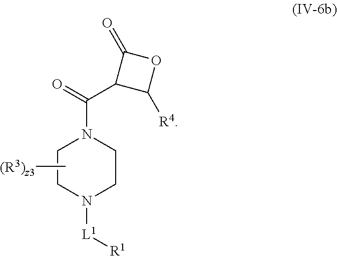

Embodiment D28. The compound of embodiment D13, having the formula:

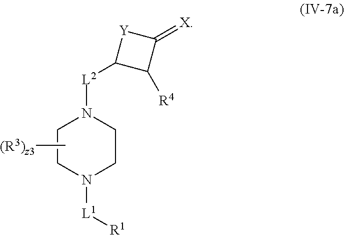

Embodiment D29. The compound of embodiment D28, having the formula:

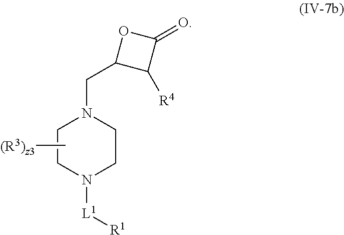

Embodiment D30. The compound of embodiment D13, having the formula:

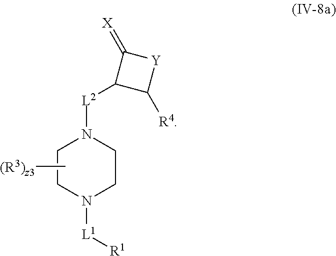

Embodiment D31. The compound of embodiment D30, having the formula:

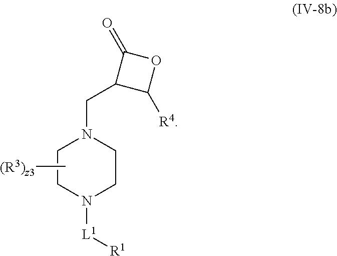

Embodiment D32. The compound of embodiment D13, having the formula:

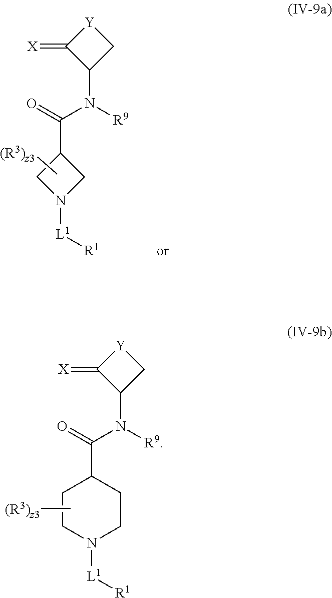

Embodiment D33. The compound of embodiment D32, having the formula:

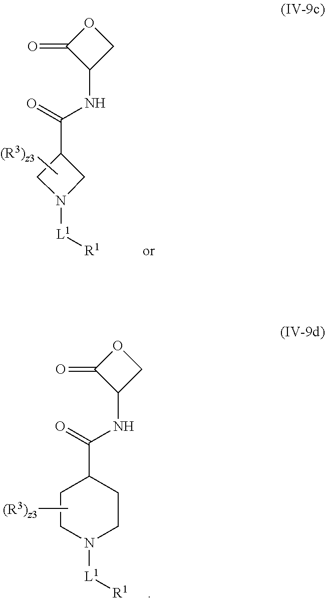

Embodiment D34. The compound of embodiment D13, having the formula:

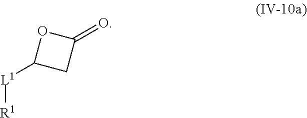

Embodiment D35. The compound of embodiment D13, having the formula:

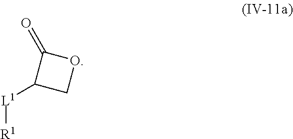

Embodiment D36. The compound of one of embodiments D13 to D35, wherein R³ is independently unsubstituted C₁-C₄ alkyl.

Embodiment D37. The compound of one of embodiments D13 to D35, wherein R³ is independently unsubstituted methyl.

Embodiment D38. The compound of one of embodiments D13 to D35, wherein two R³ substituents are joined to form a substituted or unsubstituted cycloalkyl or substituted or unsubstituted heterocycloalkyl.

Embodiment D39. The compound of one of embodiments D1 to D38, wherein
L¹ is -L¹⁰¹-L¹⁰²-L¹⁰³-;
L¹⁰¹ is connected directly to E².
L¹⁰¹ is a bond, —C(O)—, —C(O)O—, —OC(O)—, —O—, —S—, —NR¹⁰¹—, —C(O)NR¹⁰¹—, —NR¹⁰¹C(O)—, —NR¹⁰¹C(O)O—, —OC(O)NR¹⁰¹—, —NR¹⁰¹C(O)NR¹⁰¹—, —NR¹⁰¹C(NH)NR¹⁰¹—, —S(O)₂—, —NR¹⁰¹S(O)₂—, —S(O)₂NR¹⁰¹—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L¹⁰² is a bond, —C(O)—, —C(O)O—, —OC(O)—, —O—, —S—, —NR¹⁰²—, —C(O)NR¹⁰²—, —NR¹⁰²C(O)—, —NR¹⁰²C(O)O—, —OC(O)NR¹⁰²—, —NR¹⁰²C(O)NR¹⁰²—, —NR¹⁰²C(NH)NR¹⁰²—, —S(O)₂—, —NR¹⁰²S(O)₂—, —S(O)₂NR¹⁰²—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L¹⁰³ is a bond, —C(O)—, —C(O)O—, —OC(O)—, —O—, —S—, —NR¹⁰³—, —C(O)NR¹⁰³—, —NR¹⁰³C(O)—, —NR¹⁰³C(O)O—, —OC(O)NR¹⁰³—, —NR¹⁰³C(O)NR¹⁰³—, —NR¹⁰³C(NH)NR¹⁰³—, —S(O)₂—, —NR¹⁰³S(O)₂—, —S(O)₂NR¹⁰³—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; and
each R¹⁰¹, R¹⁰², and R¹⁰³ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment D40. The compound of one of embodiments D1 to D38, wherein L¹ is a bond.

Embodiment D41. The compound of one of embodiments D1 to D38, wherein L¹ is —C(O)—.

Embodiment D42. The compound of one of embodiments D1 to D38, wherein L¹ is a substituted 2 to 6 membered heteroalkylene.

Embodiment D43. The compound of one of embodiments D1 to D38, wherein L¹ is

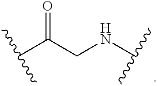 [SMILES: *NCC(*)=O]

Embodiment D44. The compound of one of embodiments D1 to D38, wherein L¹ is

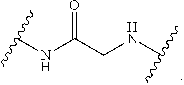 [SMILES: *NCC(=O)N*]

Embodiment D45. The compound of one of embodiments D1 to D44, wherein R¹ is -L²⁰-R²⁰.
L²⁰ is a bond, —C(O)—, —C(O)O—, —OC(O)—, —O—, —S—, —NR²⁰⁰—, —C(O)NR²⁰⁰, —NR²⁰⁰C(O)—, —NR²⁰⁰C(O)O—, —OC(O)NR²⁰⁰—, —NR²⁰⁰C(O)NR²⁰⁰—, —NR²⁰⁰C(NH)NR²⁰⁰—, —S(O)₂—, —NR²⁰⁰S(O)₂—, —S(O)₂NR²⁰⁰—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R²⁰⁰ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R²⁰ is hydrogen, halogen, —CX²⁰₃, —CHX²⁰₂, —CH₂X²⁰, —OCX²⁰₃, —OCH₂X²⁰, —OCHX²⁰₂, —CN, —SOₙ₂R^{20D}, —SOᵥ₂₀NR^{20A}R^{20B}, —NR^{20C}NR^{20A}R^{20B}, —ONR^{20A}R^{20B}, —NHC(O)NR^{20C}NR^{20A}R²⁰, —NHC(O)NR^{20A}R²⁰, —N(O)ₘ₂₀, —NR^{20A}R²⁰, —C(O)R^{20C}, —C(O)OR^{20C}, —C(O)NR^{20A}R^{20B}, —OR^{20D}, SR^{20D}, —NR^{20A}SO₂R^{20D}, —NR^{20A}C(O)R^{20C}, —NR^{20A}C(O)OR^{20C}, —NR^{20A}OR^{20C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{20A}, R^{20B}, R^{20C}, and R^{20D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{20A} and R^{20B} substituents bonded to the same nitrogen atom may optionally be joined to form a substituted or unsubstituted heterocycloalkyl or substituted or unsubstituted heteroaryl;
X²⁰ is independently —F, —Cl, —Br, or —I;
n20 is an integer from 0 to 4; and
m20 and v20 are independently 1 or 2.

Embodiment D46. The compound of one of embodiments D1 to D44, wherein R¹ is

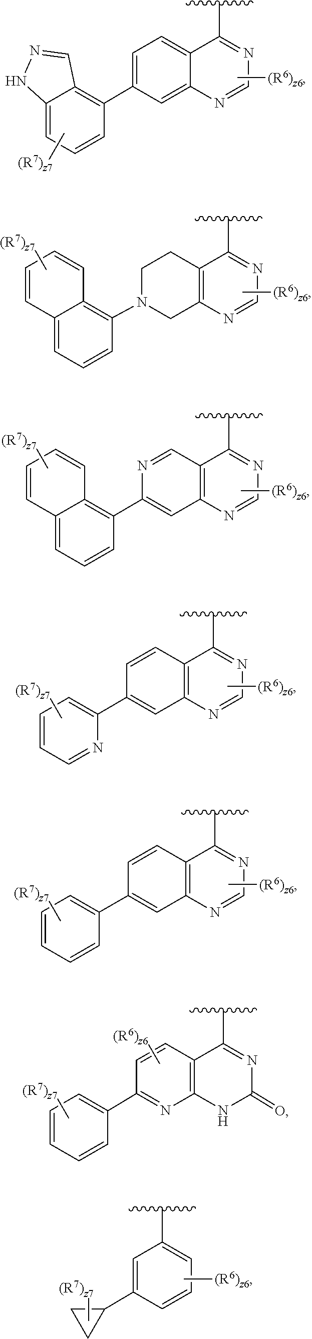

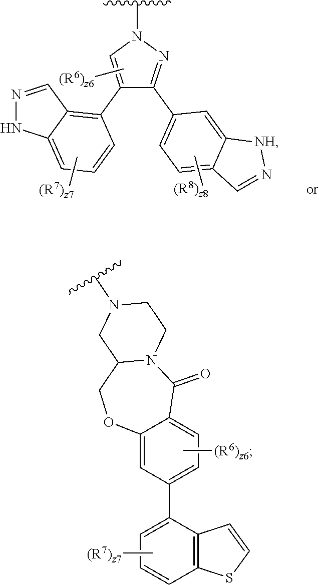

R⁶ is independently oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁷ is independently oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁸ is independently halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
z6 is an integer from 0 to 7;
z7 is an integer from 0 to 7; and
z8 is an integer from 0 to 5.

Embodiment D47. The compound of embodiment D46, wherein R⁶ is independently a halogen, —OH, unsubstituted C₁-C₄ alkyl, substituted 2 to 6 membered heteroalkyl, or substituted 5 to 6 membered heteroaryl.

Embodiment D48. The compound of embodiment D46, wherein R⁶ is independently —F, —Cl, —OH, or unsubstituted methyl.

Embodiment D49. The compound of embodiment D46, wherein R⁶ is independently a 2 to 6 membered heteroalkyl, substituted with substituted heterocycloalkyl or unsubstituted fused heterocycloalkyl.

Embodiment D50. The compound of embodiment D46, wherein R⁶ is independently a substituted pyridyl.

Embodiment D51. The compound of one of embodiments D46 to D50, wherein z6 is 1, 2, or 3.

Embodiment D52. The compound of one of embodiments D46 to D51, wherein R⁷ is independently a halogen, —CF₃, —CN, —OH, —NH₂, unsubstituted C₁-C₄ alkyl, or unsubstituted C₂-C₄ alkynyl.

Embodiment D53. The compound of one of embodiments D46 to D51, wherein R⁷ is independently —F, —Cl, —CF₃, —CN, —OH, —NH₂, unsubstituted methyl, or unsubstituted ethynyl.

Embodiment D54. The compound of one of embodiments D46 to D53, wherein z7 is 1, 2, or 3.

Embodiment D55. The compound of one of embodiments D46 to D54, wherein R⁸ is independently a halogen or unsubstituted C₁-C₄ alkyl.

Embodiment D56. The compound of one of embodiments D46 to D54, wherein R⁸ is independently —Cl or unsubstituted methyl.

Embodiment D57. The compound of one of embodiments D46 to D56, wherein z8 is 1.

Embodiment D58. The compound of one of embodiments D1 to D44, wherein R¹ is

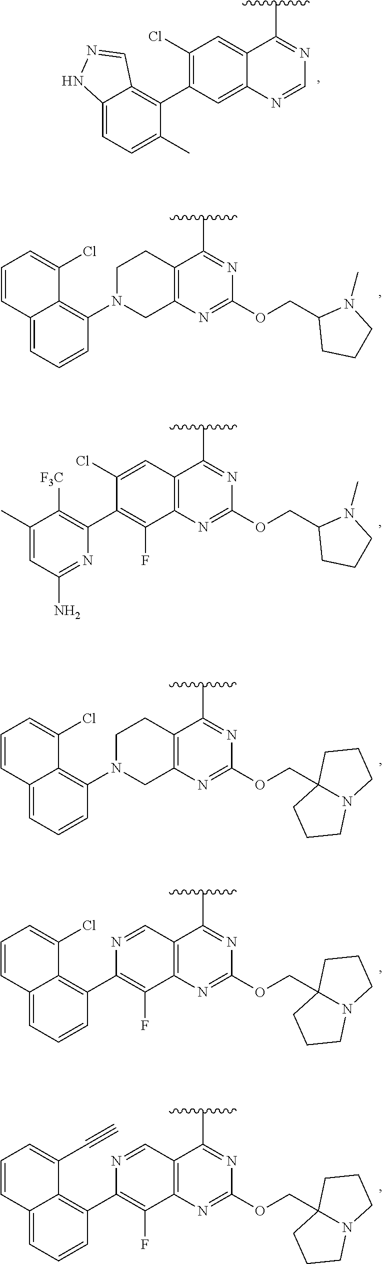 [SMILES: *c1ncnc2cc(-c3c(C)ccc4[nH]ncc34)c(Cl)cc12 ; *c1nc(OCC2CCCN2C)nc2c1CCN(c1cccc3cccc(Cl)c13)C2 ; *c1nc(OCC2CCCN2C)nc2c(F)c(-c3nc(N)cc(C)c3C(F)(F)F)c(Cl)cc12 ; *c1nc(OCC23CCCN2CCC3)nc2c1CCN(c1cccc3cccc(Cl)c13)C2 ; *c1nc(OCC23CCCN2CCC3)nc2c(F)c(-c3cccc4cccc(Cl)c34)ncc12 ; *c1nc(OCC23CCCN2CCC3)nc2c(F)c(-c3cccc4cccc(C#C)c34)ncc12]

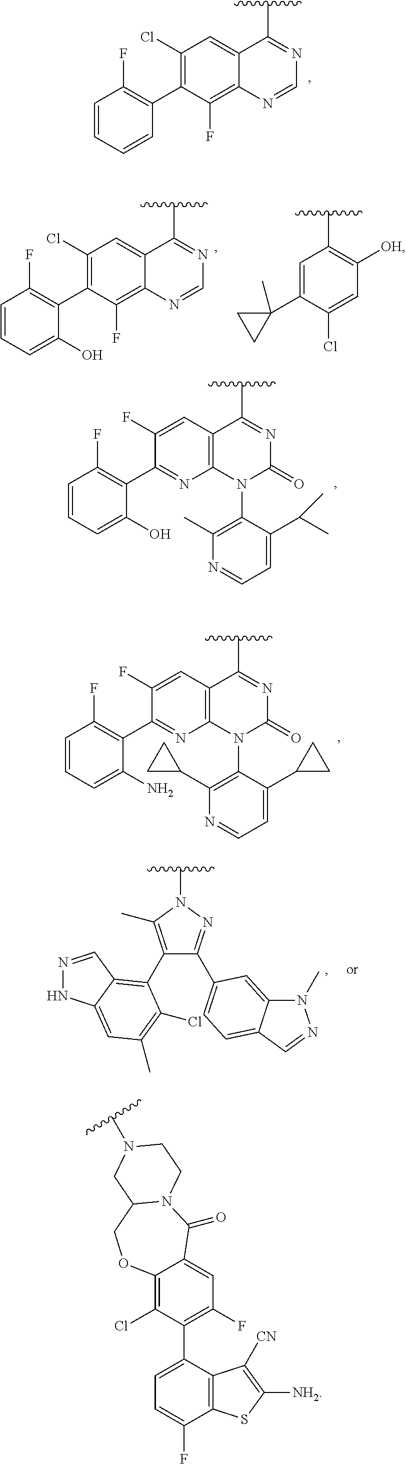

Embodiment D59. The compound of one of embodiments D1 to D11, having the formula:

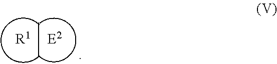

Embodiment D60. The compound of embodiment D59, having the formula:

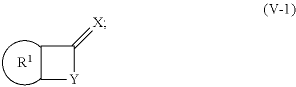

wherein
X is O or S;
Y is O, S, or NR²; and
R² is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH2, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment D61. The compound of embodiment D60, having the formula:

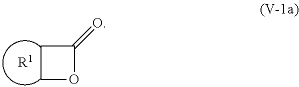

Embodiment D62. The compound of one of embodiments D59 to D61, wherein R¹ is

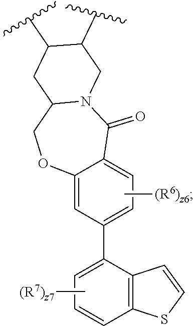

wherein
R⁶ is independently oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁷ is independently halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
z6 is an integer from 0 to 3; and
z7 is an integer from 0 to 5.

Embodiment D63. The compound of embodiment D62, wherein R¹ is

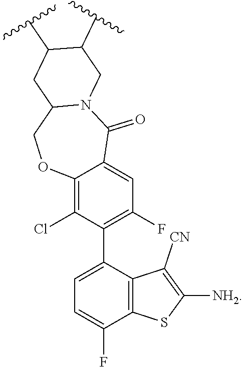 [SMILES: *C1CC2COc3c(cc(F)c(-c4ccc(F)c5sc(N)c(C#N)c45)c3Cl)C(=O)N2CC1*]

Embodiment D64. The compound of one of embodiments D1 to D11, having the formula:

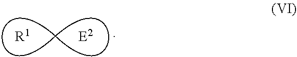

Embodiment D65. The compound of embodiment D64, having the formula:

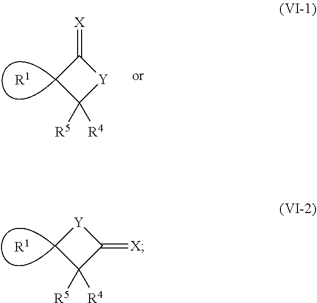

wherein
X is O or S;
Y is O, S, or NR²;
R² is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁴ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and
R⁵ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment D66. The compound of embodiment D65, having the formula:

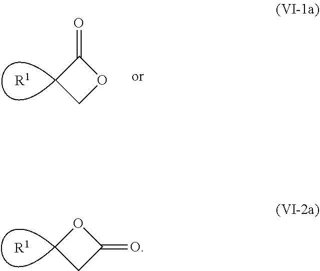

Embodiment D67. The compound of one of embodiments D64 to D66, wherein R¹ is

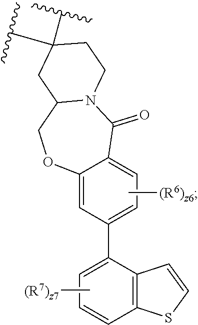

wherein
R⁶ is independently oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁷ is independently halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
z6 is an integer from 0 to 3; and
z7 is an integer from 0 to 5.

Embodiment D68. The compound of embodiment D67, wherein R¹ is

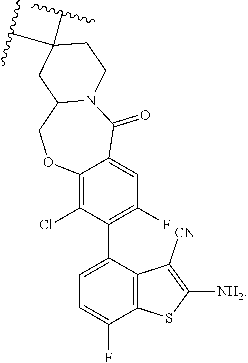 [SMILES: *C1(*)CCN2C(=O)c3cc(F)c(-c4ccc(F)c5sc(N)c(C#N)c45)c(Cl)c3OCC2C1]

Embodiment D69. A pharmaceutical composition comprising a pharmaceutically acceptable excipient and a compound of one of embodiments D1 to D68, or a pharmaceutically acceptable salt thereof.

Embodiment D70. A method of treating cancer in a subject in need thereof, said method comprising administering to the subject in need thereof a therapeutically effective amount of a compound of one of embodiments D1 to D68, or a pharmaceutically acceptable salt thereof.

Embodiment D71. The method of embodiment D70, wherein the cancer is rectal carcinoma, colorectal adenocarcinoma, colorectal carcinoma, non-small cell lung carcinoma, squamous cell lung carcinoma, myelodysplastic syndrome, or acute myeloid leukemia.

Embodiment D72. A method of treating a K-Ras(G12D)-associated disease in a subject in need thereof, said method comprising administering to the subject in need thereof a therapeutically effective amount of a compound of one of embodiments D1 to D68, or a pharmaceutically acceptable salt thereof.

Embodiment D73. A method of treating an H-Ras(G12D)-associated disease in a subject in need thereof, said method comprising administering to the subject in need thereof a therapeutically effective amount of a compound of one of embodiments D1 to D68, or a pharmaceutically acceptable salt thereof.

Embodiment D74. A method of treating an N-Ras(G12D)-associated disease in a subject in need thereof, said method comprising administering to the subject in need thereof a therapeutically effective amount of a compound of one of embodiments D1 to D68, or a pharmaceutically acceptable salt thereof.

Embodiment D75. A method of modulating the level of activity of a K-Ras protein in a cell, said method comprising contacting the cell with an effective amount of a compound of one of embodiments D1 to D68, or a pharmaceutically acceptable salt thereof.

Embodiment D76. The method of embodiment D75, wherein said modulating of said activity comprises modulating GTPase activity, nucleotide exchange, differential GDP or GTP binding, effector protein binding, effector protein activation, guanine exchange factor (GEF) binding, GEF-facilitated nucleotide exchange, phosphate release, nucleotide release, nucleotide binding, K-Ras subcellular localization, K-Ras post-translational processing, or K-Ras post-translational modifications.

Embodiment D77. The method of one of embodiments D75 to D76, wherein said modulating is increasing the activity of said K-Ras protein.

Embodiment D78. The method of one of embodiments D75 to D76, wherein said modulating is reducing the activity of said K-Ras protein.

Embodiment D79. The method of one of embodiments D75 to D78, wherein said K-Ras protein is a human K-Ras protein.

Embodiment D80. The method of embodiment 79, wherein said human K-Ras protein contains a G12D mutation.

Embodiment D81. A method of modulating the level of activity of an H-Ras protein in a cell, said method comprising contacting the cell with an effective amount of a compound of one of embodiments D1 to D68, or a pharmaceutically acceptable salt thereof.

Embodiment D82. The method of embodiment D81, wherein said modulating of said activity comprises modulating GTPase activity, nucleotide exchange, differential GDP or GTP binding, effector protein binding, effector protein activation, guanine exchange factor (GEF) binding, GEF-facilitated nucleotide exchange, phosphate release, nucleotide release, nucleotide binding, H-Ras subcellular localization, H-Ras post-translational processing, or H-Ras post-translational modifications.

Embodiment D83. The method of one of embodiments D81 to D82, wherein said modulating is increasing the activity of said H-Ras protein.

Embodiment D84. The method of one of embodiments D81 to D82, wherein said modulating is reducing the activity of said H-Ras protein.

Embodiment D85. The method of one of embodiments D81 to D84, wherein said H-Ras protein is a human H-Ras protein.

Embodiment D86. The method of embodiment D85, wherein said human H-Ras protein contains a G12D mutation.

Embodiment D87. A method of modulating the level of activity of an N-Ras protein in a cell, said method comprising contacting the cell with an effective amount of a compound of one of embodiments D1 to D68, or a pharmaceutically acceptable salt thereof.

Embodiment D88. The method of embodiment D87, wherein said modulating of said activity comprises modulating GTPase activity, nucleotide exchange, differential GDP or GTP binding, effector protein binding, effector protein activation, guanine exchange factor (GEF) binding, GEF-facilitated nucleotide exchange, phosphate release, nucleotide release, nucleotide binding, N-Ras subcellular localization, N-Ras post-translational processing, or N-Ras post-translational modifications.

Embodiment D89. The method of one of embodiments D87 to D88, wherein said modulating is increasing the activity of said N-Ras protein.

Embodiment D90. The method of one of embodiments D87 to D88, wherein said modulating is reducing the activity of said N-Ras protein.

Embodiment D91. The method of one of embodiments D87 to D90, wherein said N-Ras protein is a human K-Ras protein.

Embodiment D92. The method of embodiment D91, wherein said human N-Ras protein contains a G12D mutation.

Embodiment D93. A K-Ras protein covalently bound to a compound of one of embodiments D1 to D68, or a pharmaceutically acceptable salt thereof, wherein said compound is covalently bound to an aspartate residue of said K-Ras protein.

Embodiment D94. The covalently modified K-Ras protein of embodiment D93, wherein said compound is reversibly covalently bound to an aspartate residue of said K-Ras protein.

Embodiment D95. The covalently modified K-Ras protein of embodiment D93, wherein said compound is irreversibly covalently bound to an aspartate residue of said K-Ras protein.

Embodiment D96. The covalently modified K-Ras protein of one of embodiments D93 to D95, wherein said covalently modified K-Ras protein has a modulated activity relative to a control, wherein said activity is selected from GTPase activity, nucleotide exchange, differential GDP or GTP binding, effector protein binding, effector protein activation, guanine exchange factor (GEF) binding, GEF-facilitated nucleotide exchange, phosphate release, nucleotide release, nucleotide binding, K-Ras subcellular localization, K-Ras post-translational processing, and K-Ras post-translational modifications.

Embodiment D97. The covalently modified K-Ras protein of one of embodiments D93 to D96, wherein said K-Ras protein contains a G12D mutation.

Embodiment D98. The covalently modified K-Ras protein of embodiment D97, wherein said compound is covalently bonded to aspartate residue 12.

Embodiment D99. An H-Ras protein covalently bound to a compound of one of embodiments D1 to D68, or a pharmaceutically acceptable salt thereof, wherein said compound is covalently bound to an aspartate residue of said H-Ras protein.

Embodiment D100. The covalently modified H-Ras protein of embodiment D99, wherein said compound is reversibly covalently bound to an aspartate residue of said H-Ras protein.

Embodiment D101. The covalently modified H-Ras protein of embodiment D99, wherein said compound is irreversibly covalently bound to an aspartate residue of said H-Ras protein.

Embodiment D102. The covalently modified H-Ras protein of one of embodiments D99 to D101, wherein said covalently modified H-Ras protein has a modulated activity relative to a control, wherein said activity is selected from GTPase activity, nucleotide exchange, differential GDP or GTP binding, effector protein binding, effector protein activation, guanine exchange factor (GEF) binding, GEF-facilitated nucleotide exchange, phosphate release, nucleotide release, nucleotide binding, H-Ras subcellular localization, H-Ras post-translational processing, and H-Ras post-translational modifications.

Embodiment D103. The covalently modified H-Ras protein of one of embodiments D99 to D102, wherein said H-Ras protein contains a G12D mutation.

Embodiment D104. The covalently modified H-Ras protein of embodiment D103, wherein said compound is covalently bonded to aspartate residue 12.

Embodiment D105. An N-Ras protein covalently bound to a compound of one of embodiments D1 to D68, or a pharmaceutically acceptable salt thereof, wherein said compound is covalently bound to an aspartate residue of said N-Ras protein.

Embodiment D106. The covalently modified N-Ras protein of embodiment D105, wherein said compound is reversibly covalently bound to an aspartate residue of said N-Ras protein.

Embodiment D107. The covalently modified N-Ras protein of embodiment D105, wherein said compound is irreversibly covalently bound to an aspartate residue of said N-Ras protein.

Embodiment D108. The covalently modified N-Ras protein of one of embodiments D105 to D107, wherein said covalently modified H-Ras protein has a modulated activity relative to a control, wherein said activity is selected from GTPase activity, nucleotide exchange, differential GDP or GTP binding, effector protein binding, effector protein activation, guanine exchange factor (GEF) binding, GEF-facilitated nucleotide exchange, phosphate release, nucleotide release, nucleotide binding, N-Ras subcellular localization, N-Ras post-translational processing, and N-Ras post-translational modifications.

Embodiment D109. The covalently modified N-Ras protein of one of embodiments D105 to D108, wherein said H-Ras protein contains a G12D mutation.

Embodiment D110. The covalently modified N-Ras protein of embodiment D109, wherein said compound is covalently bonded to aspartate residue 12.

### VII. Additional Embodiments

Embodiment 1. A compound, or a pharmaceutically acceptable salt thereof, having the formula:

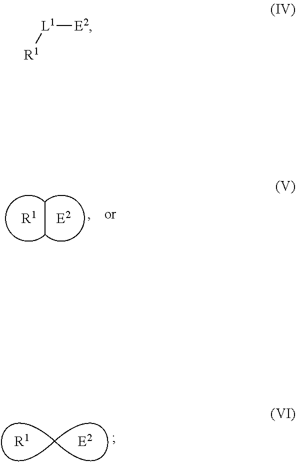

wherein
R¹ is a Switch II Binding Pocket binding moiety;
L¹ is a bond or divalent linker; and
E² is an electrophilic moiety capable of forming a covalent bond with a Switch II GTPase protein aspartate residue or a Switch II GTPase protein glutamate residue.

Embodiment 2. The compound of embodiment 1, wherein E² is an electrophilic moiety capable of forming a covalent bond with a Switch II GTPase protein aspartate residue.

Embodiment 3. The compound of embodiment 2, wherein the Switch II GTPase protein aspartate residue is a natural Switch II GTPase protein aspartate residue.

Embodiment 4. The compound of embodiment 2, wherein the Switch II GTPase protein aspartate residue is a mutant Switch II GTPase protein aspartate residue.

Embodiment 5. The compound of embodiment 4, wherein the mutant Switch II GTPase protein aspartate residue is aspartate residue 12 of K-Ras(G12D), H-Ras(G12D), or N-Ras(G12D).

Embodiment 6. The compound of embodiment 4, wherein the mutant Switch II GTPase protein aspartate residue is aspartate residue 13 of K-Ras(G13D), H-Ras(G13D), or N-Ras(G13D).

Embodiment 7. The compound of embodiment 1, wherein E² is an electrophilic moiety capable of forming a covalent bond with a Switch II GTPase protein glutamate residue.

Embodiment 8. The compound of embodiment 7, wherein the Switch II GTPase protein glutamate residue is a natural Switch II GTPase protein glutamate residue.

Embodiment 9. The compound of embodiment 7, wherein the Switch II GTPase protein glutamate residue is a mutant Switch II GTPase protein glutamate residue.

Embodiment 10. The compound of one of embodiments 1 to 9, wherein E² comprises a β-lactone.

Embodiment 11. The compound of one of embodiments 1 to 9, wherein E² comprises a β-lactam.

Embodiment 12. The compound of one of embodiments 1 to 11, having the formula:

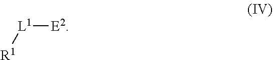

Embodiment 13. The compound of embodiment 12, having the formula:

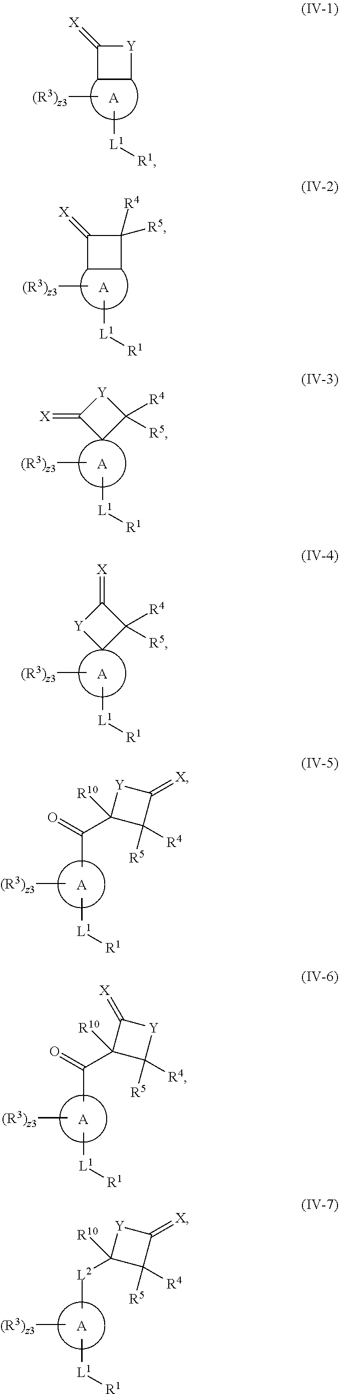

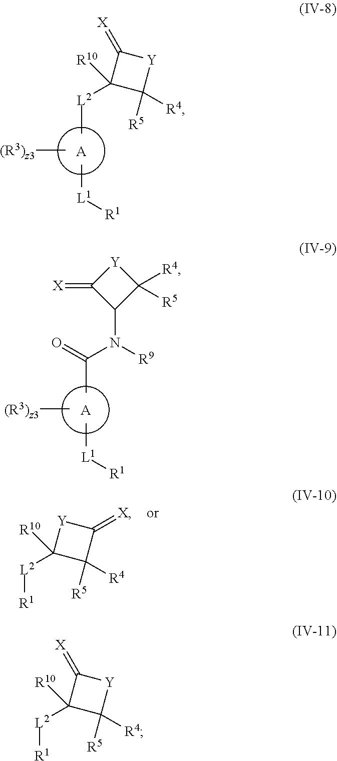

wherein
Ring A is a cycloalkyl or heterocycloalkyl;
L² is unsubstituted C₁-C₄ alkylene;
X is O or S;
Y is O, S, or NR²;
R² is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³ is independently oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R³ substituents may optionally be joined to form a substituted or unsubstituted cycloalkyl, substituted or unsubstituted heterocycloalkyl, substituted or unsubstituted aryl, or substituted or unsubstituted heteroaryl;
z3 is an integer from 0 to 10;
R⁴ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁵ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁹ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and
R¹⁰ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment 14. The compound of embodiment 13, wherein Ring A is a 5 to 6 membered heterocycloalkyl.

Embodiment 15. The compound of embodiment 13, wherein Ring A is a piperidinyl, pyrrolidinyl, or piperazinyl.

Embodiment 16. The compound of embodiment 13, having the formula:

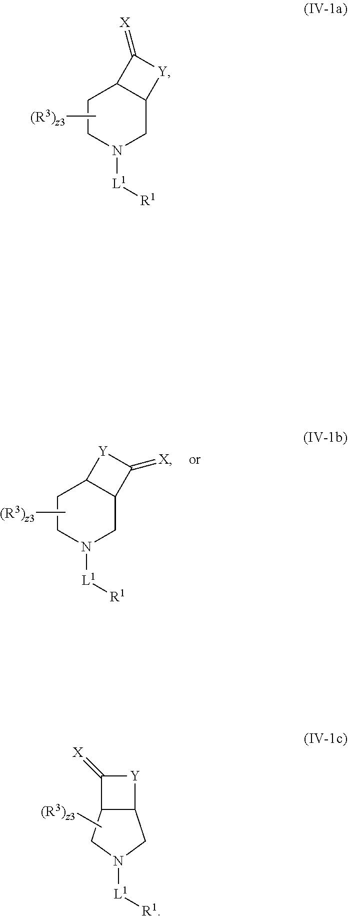

Embodiment 17. The compound of embodiment 16, having the formula:

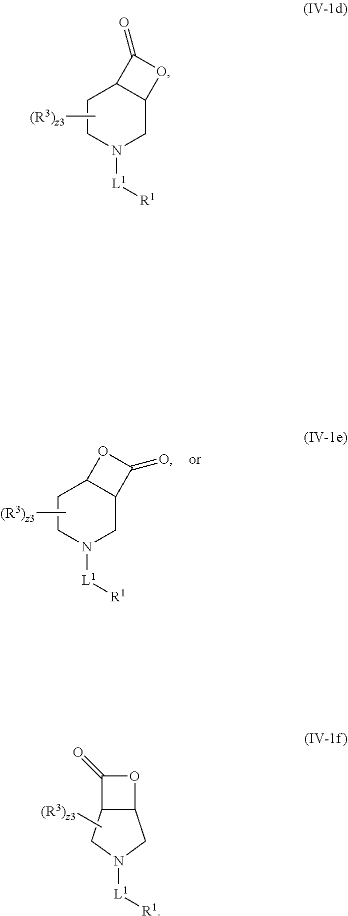

Embodiment 18. The compound of embodiment 13, having the formula:

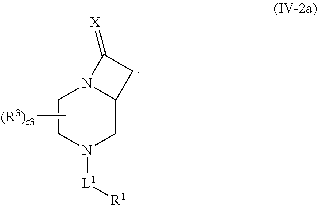

Embodiment 19. The compound of embodiment 18, having the formula:

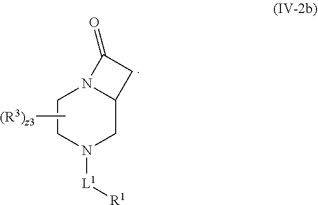

Embodiment 20. The compound of embodiment 13, having the formula:

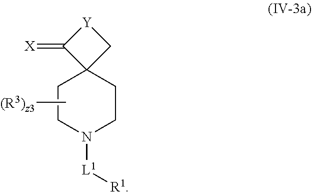

Embodiment 21. The compound of embodiment 20, having the formula:

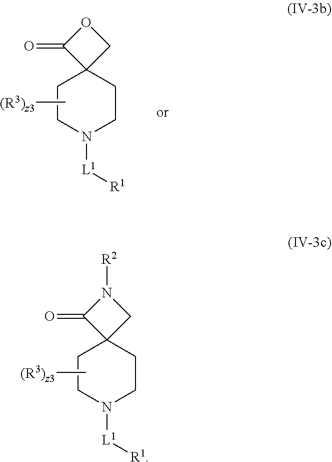

Embodiment 22. The compound of embodiment 13, having the formula:

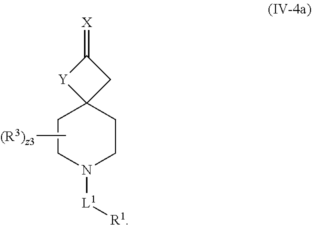

Embodiment 23. The compound of embodiment 22, having the formula:

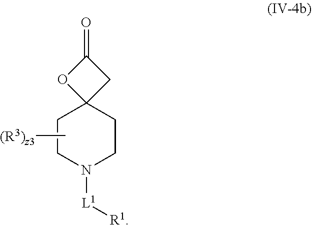

Embodiment 24. The compound of embodiment 13, having the formula:

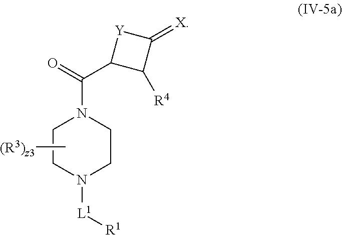

Embodiment 25. The compound of embodiment 24, having the formula:

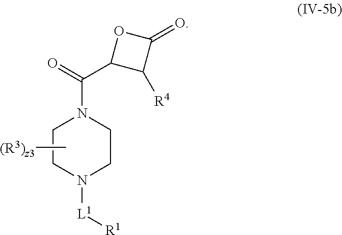

Embodiment 26. The compound of one of embodiments 24 to 25, having the formula:

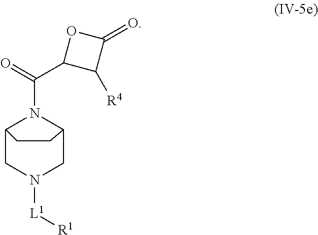

Embodiment 27. The compound of embodiment 13, having the formula:

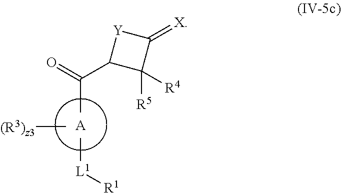

Embodiment 28. The compound of embodiment 27, having the formula:

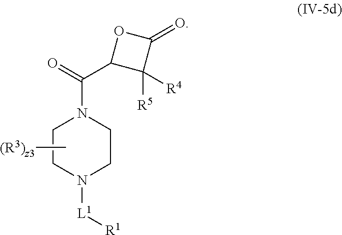

Embodiment 29. The compound of one of embodiments 27 to 28, having the formula:

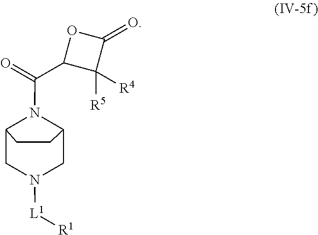

Embodiment 30. The compound of embodiment 13, having the formula:

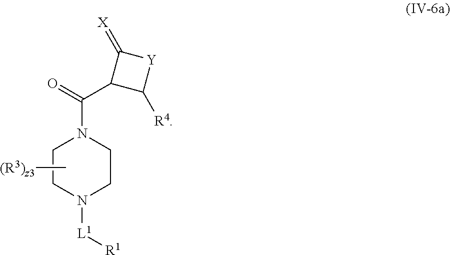

Embodiment 31. The compound of embodiment 30, having the formula:

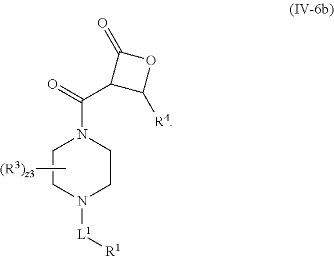

Embodiment 32. The compound of embodiment 13, having the formula:

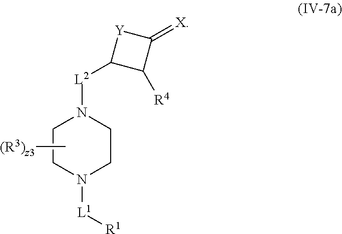

Embodiment 33. The compound of embodiment 32, having the formula:

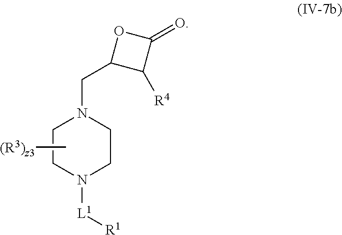

Embodiment 34. The compound of embodiment 13, having the formula:

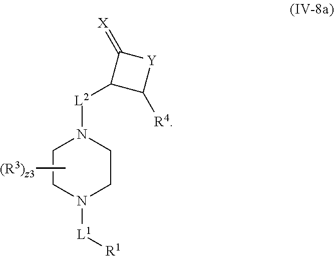

Embodiment 35. The compound of embodiment 34, having the formula:

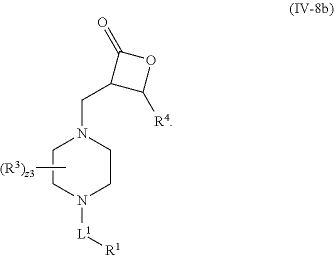

Embodiment 36. The compound of embodiment 13, having the formula:

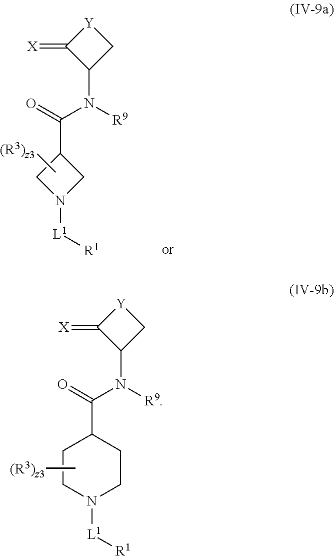

Embodiment 37. The compound of embodiment 36, having the formula:

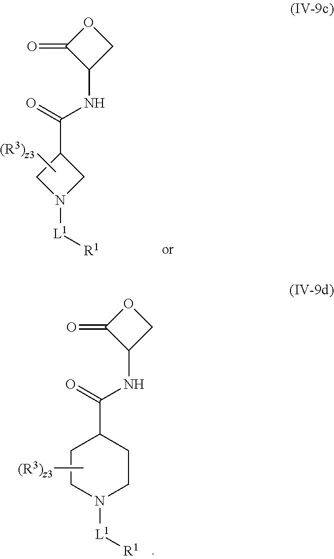

Embodiment 38. The compound of embodiment 13, having the formula:

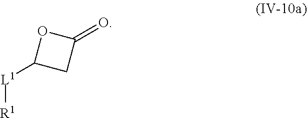

Embodiment 39. The compound of embodiment 13, having the formula:

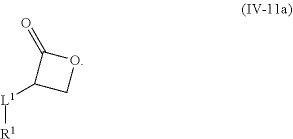

Embodiment 40. The compound of one of embodiments 13 to 25, 27, 28, and 30 to 37, wherein R³ is independently unsubstituted C₁-C₄ alkyl.

Embodiment 41. The compound of one of embodiments 13 to 25, 27, 28, and 30 to 37, wherein R³ is independently unsubstituted methyl.

Embodiment 42. The compound of one of embodiments 13 to 25, 27, 28, and 30 to 37, wherein two R³ substituents are joined to form a substituted or unsubstituted cycloalkyl or substituted or unsubstituted heterocycloalkyl.

Embodiment 43. The compound of one of embodiments 1 to 42, wherein
L¹ is -L¹⁰¹-L¹⁰²-L¹⁰³-;
L¹⁰¹ is connected directly to E²;
L¹⁰¹ is a bond, —C(O)—, —C(O)O—, —OC(O)—, —O—, —S—, —NR¹⁰¹—, —C(O)NR¹⁰¹—, —NR¹⁰¹C(O)—, —NR¹⁰¹C(O)O—, —OC(O)NR¹⁰¹—, —NR¹⁰¹C(O)NR¹⁰¹—, —NR¹⁰¹C(NH)NR¹⁰¹—, —S(O)₂—, —NR¹⁰¹S(O)₂—, —S(O)₂NR¹⁰¹—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L¹⁰² is a bond, —C(O)—, —C(O)O—, —OC(O)—, —O—, —S—, —NR¹⁰²—, —C(O)NR¹⁰²—, —NR¹⁰²C(O)—, —NR¹⁰²C(O)O—, —OC(O)NR¹⁰²—, —NR¹⁰²C(O)NR¹⁰²—, —NR¹⁰²C(NH)NR¹⁰²—, —S(O)₂—, —NR¹⁰²S(O)₂—, —S(O)₂NR¹⁰²—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L¹⁰³ is a bond, —C(O)—, —C(O)O—, —OC(O)—, —O—, —S—, —NR¹⁰³—, —C(O)NR¹⁰³—, —NR¹⁰³C(O)—, —NR¹⁰³C(O)O—, —OC(O)NR¹⁰³—, —NR¹⁰³C(O)NR¹⁰³—, —NR¹⁰³C(NH)NR¹⁰³—, —S(O)₂—, —NR¹⁰³S(O)₂—, —S(O)₂NR¹⁰³—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; and
each R¹⁰¹, R¹⁰², and R¹⁰³ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment 44. The compound of one of embodiments 1 to 42, wherein L¹ is a bond.

Embodiment 45. The compound of one of embodiments 1 to 42, wherein L¹ is —C(O)—.

Embodiment 46. The compound of one of embodiments 1 to 42, wherein L¹ is a substituted 2 to 6 membered heteroalkylene.

Embodiment 47. The compound of one of embodiments 1 to 42, wherein L¹ is

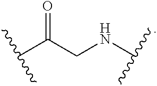 [SMILES: *NCC(*)=O]

Embodiment 48. The compound of one of embodiments 1 to 42, wherein L¹ is

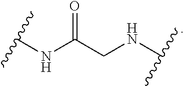 [SMILES: *NCC(=O)N*]

Embodiment 49. The compound of one of embodiments 1 to 48, wherein R¹ is -L²⁰-R²⁰;
L²⁰ is a bond, —C(O)—, —C(O)O—, —OC(O)—, —O—, —S—, —NR²⁰⁰—, —C(O)NR²⁰⁰—, —NR²⁰⁰C(O)—, —NR²⁰⁰C(O)O—, —OC(O)NR²⁰⁰—, —NR²⁰⁰C(O)NR²⁰⁰—, —NR²⁰⁰C(NH)NR²⁰⁰—, —S(O)₂—, —NR²⁰⁰S(O)₂—, —S(O)₂NR²⁰⁰—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R²⁰⁰ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R²⁰ is hydrogen, halogen, —CX²⁰₃, —CHX²⁰₂, —CH₂X²⁰, —OCX²⁰₃, —OCH₂X²⁰, —OCHX²⁰₂, —CN, —SOₙ₂₀R^{20D}, SOᵥ₂₀NR^{20A}R^{20B}, —NR^{20C}NR^{20A}R^{20B}, —ONR^{20A}R^{20B}, —NHC(O)NR^{20C}NR^{20A}R^{20B}, —NHC(O)NR^{20A}R²⁰, —N(O)ₘ₂₀, —NR^{20A}R^{20B}, —C(O)R^{20C}, —C(O)OR^{20C}, —C(O)NR^{20A}R^{20B}, —OR^{20D}, —SR^{20D}, —NR^{20A}SO₂R^{20D}, —NR^{20A}C(O)R^{20C}, —NR^{20A}C(O)OR^{20C}, —NR^{20A}OR^{20C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{20A}, R^{20B}, R^{20C}, and R^{20D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{20A} and R^{20B} substituents bonded to the same nitrogen atom may optionally be joined to form a substituted or unsubstituted heterocycloalkyl or substituted or unsubstituted heteroaryl;
X²⁰ is independently —F, —Cl, —Br, or —I;
n20 is an integer from 0 to 4; and
m20 and v20 are independently 1 or 2.

Embodiment 50. The compound of one of embodiments 1 to 48, wherein R¹ is

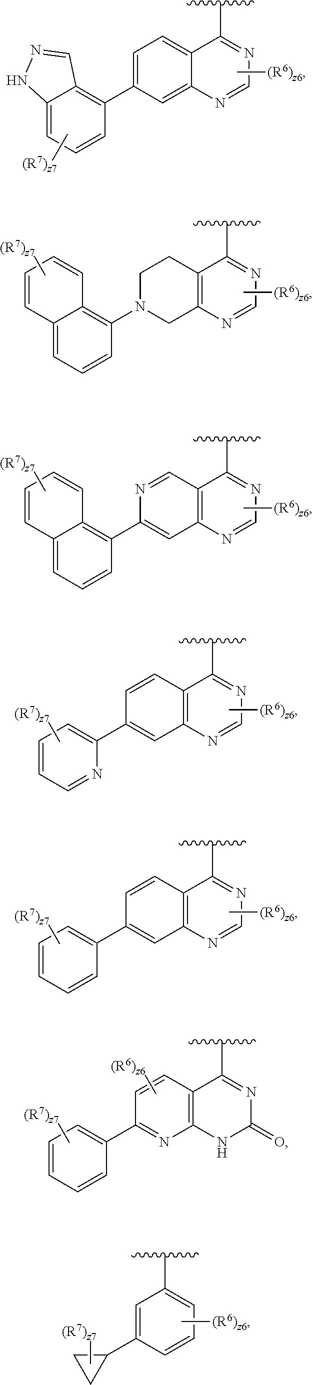

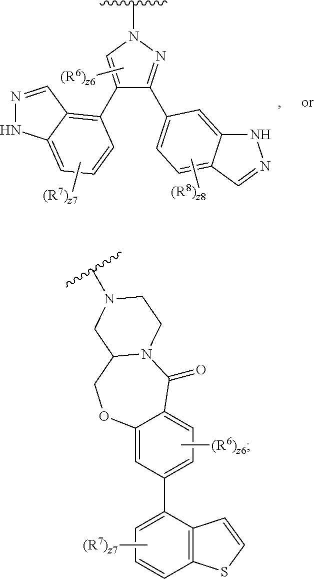

R⁶ is independently oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁷ is independently oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁸ is independently halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
z6 is an integer from 0 to 7;
z7 is an integer from 0 to 7; and
z8 is an integer from 0 to 5.

Embodiment 51. The compound of embodiment 50, wherein R⁶ is independently a halogen, —OH, unsubstituted C₁-C₄ alkyl, substituted 2 to 6 membered heteroalkyl, or substituted 5 to 6 membered heteroaryl.

Embodiment 52. The compound of embodiment 50, wherein R⁶ is independently —F, —Cl, —OH, or unsubstituted methyl.

Embodiment 53. The compound of embodiment 50, wherein R⁶ is independently a 2 to 6 membered heteroalkyl, substituted with substituted heterocycloalkyl or substituted or unsubstituted fused heterocycloalkyl.

Embodiment 54. The compound of embodiment 50, wherein R⁶ is independently

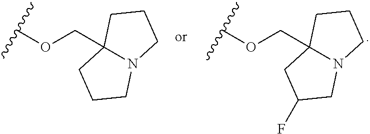 [SMILES: *OCC12CCCN1CCC2 ; *OCC12CCCN1CC(F)C2]

Embodiment 55. The compound of embodiment 50, wherein R⁶ is independently a substituted pyridyl.

Embodiment 56. The compound of one of embodiments 50 to 55, wherein z6 is 1, 2, or 3.

Embodiment 57. The compound of one of embodiments 50 to 56, wherein R⁷ is independently a halogen, —CF₃, —CN, —OH, —NH₂, unsubstituted C₁-C₄ alkyl, or unsubstituted C₂-C₄ alkynyl.

Embodiment 58. The compound of one of embodiments 50 to 56, wherein R⁷ is independently —F, —Cl, —CF₃, —CN, —OH, —NH₂, unsubstituted methyl, or unsubstituted ethynyl.

Embodiment 59. The compound of one of embodiments 50 to 58, wherein z7 is 1, 2, or 3.

Embodiment 60. The compound of one of embodiments 50 to 59, wherein R⁸ is independently a halogen or unsubstituted C₁-C₄ alkyl.

Embodiment 61. The compound of one of embodiments 50 to 59, wherein R⁸ is independently —Cl or unsubstituted methyl.

Embodiment 62. The compound of one of embodiments 50 to 61, wherein z8 is 1.

Embodiment 63. The compound of one of embodiments 1 to 48, wherein R¹ is

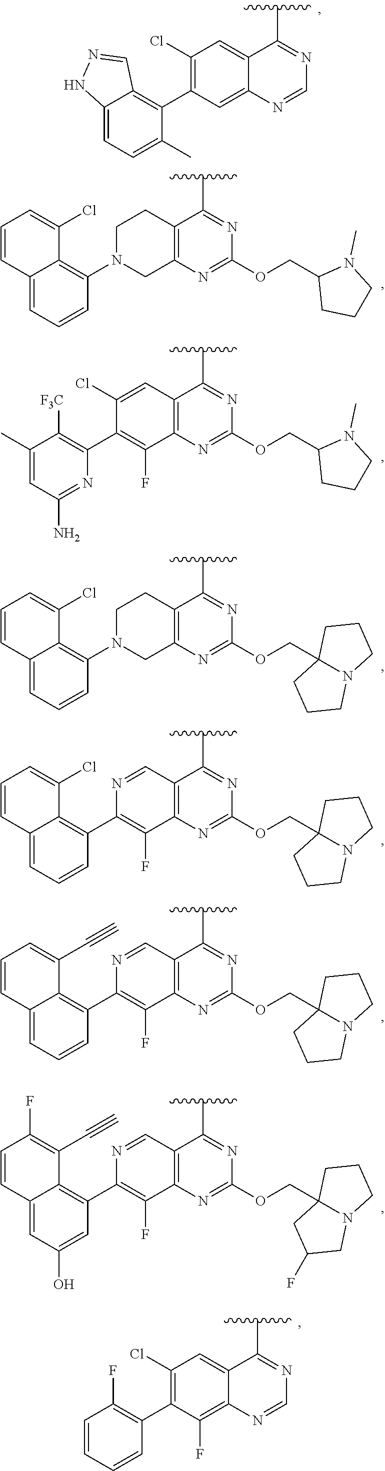

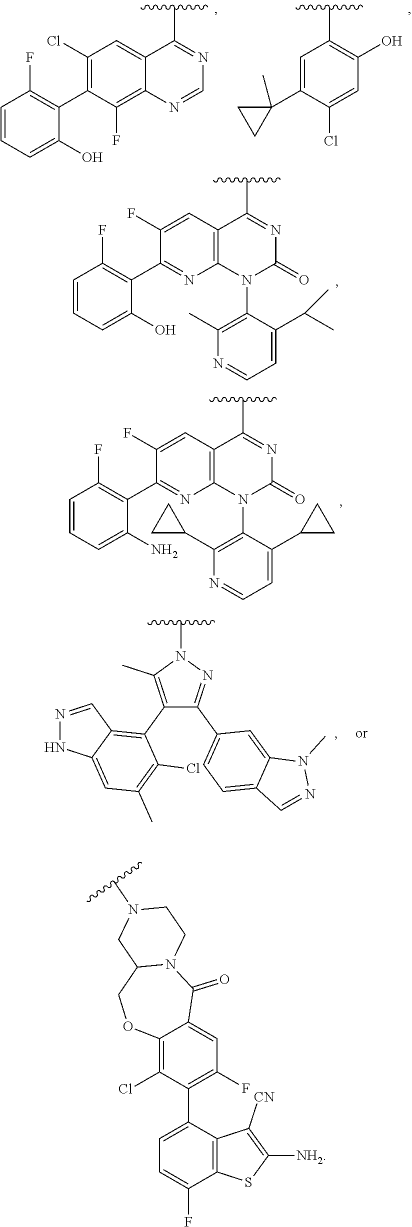

Embodiment 64. The compound of embodiment 50, having the formula:

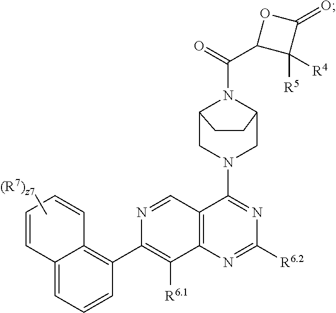

wherein
R⁴ and R⁵ are independently hydrogen or unsubstituted C₁-C₄ alkyl;
R^{6.1} is halogen;
R^{6.2} is —O—(C₁-C₄ alkyl), wherein the C₁-C₄ alkyl is substituted with a 5 to 8 membered heterocycloalkyl optionally substituted with halogen or unsubstituted C₁-C₃ alkyl;
R⁷ is independently halogen, —OH, or unsubstituted C₂ alkynyl; and
z7 is 1, 2, or 3.

Embodiment 65. The compound of one of embodiments 1 to 11, having the formula:

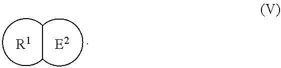

Embodiment 66. The compound of embodiment 65, having the formula:

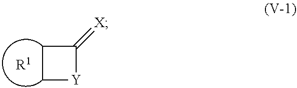

wherein
X is O or S;
Y is O, S, or NR²; and
R² is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment 67. The compound of embodiment 66, having the formula:

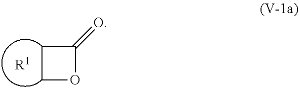

Embodiment 68. The compound of one of embodiments 65 to 67, wherein R¹ is

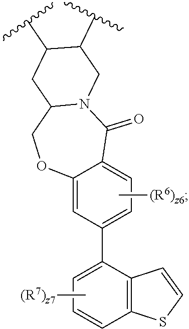

wherein
R⁶ is independently oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁷ is independently halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
z6 is an integer from 0 to 3; and
z7 is an integer from 0 to 5.

Embodiment 69. The compound of embodiment 68, wherein R¹ is

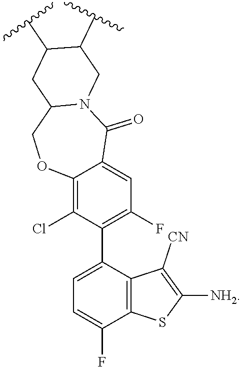 [SMILES: *C1CC2COc3c(cc(F)c(-c4ccc(F)c5sc(N)c(C#N)c45)c3Cl)C(=O)N2CC1*]

Embodiment 70. The compound of one of embodiments 1 to 11, having the formula:

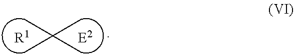

Embodiment 71. The compound of embodiment 70, having the formula:

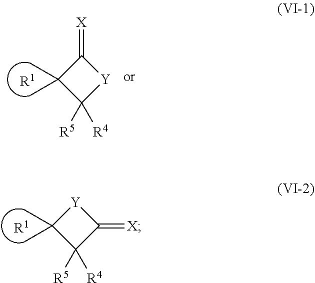

wherein
X is O or S;
Y is O, S, or NR²;
R² is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁴ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and
R⁵ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment 72. The compound of embodiment 71, having the formula:

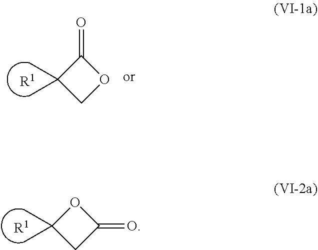

Embodiment 73. The compound of one of embodiments 70 to 72, wherein R¹ is

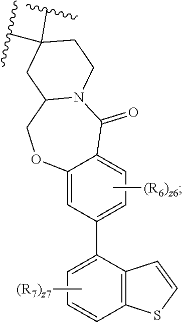

wherein
R⁶ is independently oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁷ is independently halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
z6 is an integer from 0 to 3; and
z7 is an integer from 0 to 5.

Embodiment 74. The compound of embodiment 73, wherein R¹ is

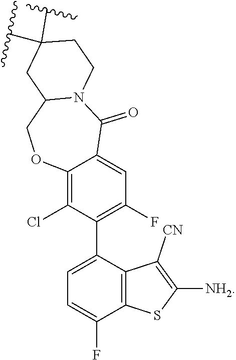 [SMILES: *C1(*)CCN2C(=O)c3cc(F)c(-c4ccc(F)c5sc(N)c(C#N)c45)c(Cl)c3OCC2C1]

Embodiment 75. The compound of one of embodiments 1 to 10 and 12 to 15, having the formula:

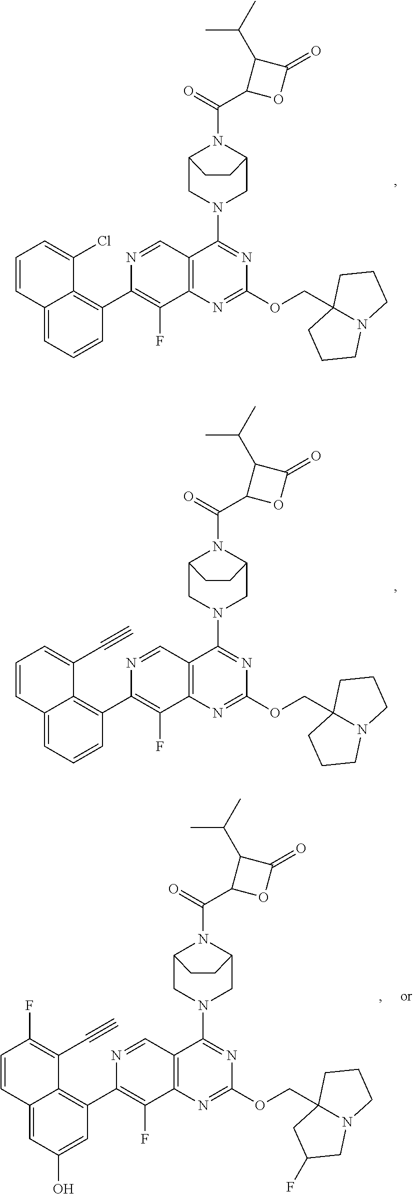 [SMILES: CC(C)C1C(=O)OC1C(=O)N1C2CCC1CN(c1nc(OCC34CCCN3CCC4)nc3c(F)c(-c4cccc5cccc(Cl)c45)ncc13)C2 ; C#Cc1cccc2cccc(-c3ncc4c(N5CC6CCC(C5)N6C(=O)C5OC(=O)C5C(C)C)nc(OCC56CCCN5CCC6)nc4c3F)c12 ; C#Cc1c(F)ccc2cc(O)cc(-c3ncc4c(N5CC6CCC(C5)N6C(=O)C5OC(=O)C5C(C)C)nc(OCC56CCCN5CC(F)C6)nc4c3F)c12]

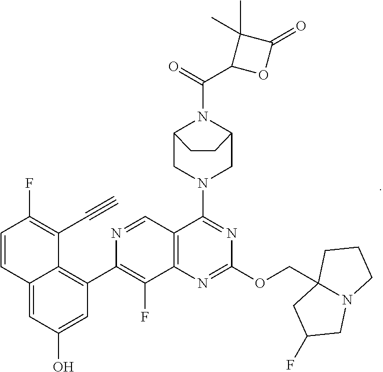 [SMILES: C#Cc1c(F)ccc2cc(O)cc(-c3ncc4c(N5CC6CCC(C5)N6C(=O)C5OC(=O)C5(C)C)nc(OCC56CCCN5CC(F)C6)nc4c3F)c12]

Embodiment 76. A pharmaceutical composition comprising a pharmaceutically acceptable excipient and a compound of one of embodiments 1 to 75, or a pharmaceutically acceptable salt thereof.

Embodiment 77. A method of treating cancer in a subject in need thereof, said method comprising administering to the subject in need thereof a therapeutically effective amount of a compound of one of embodiments 1 to 75, or a pharmaceutically acceptable salt thereof.

Embodiment 78. The method of embodiment 77, wherein the cancer is rectal carcinoma, colorectal adenocarcinoma, colorectal carcinoma, non-small cell lung carcinoma, squamous cell lung carcinoma, myelodysplastic syndrome, or acute myeloid leukemia.

Embodiment 79. A method of treating a K-Ras(G12D)-associated disease in a subject in need thereof, said method comprising administering to the subject in need thereof a therapeutically effective amount of a compound of one of embodiments 1 to 75, or a pharmaceutically acceptable salt thereof.

Embodiment 80. A method of treating an H-Ras(G12D)-associated disease in a subject in need thereof, said method comprising administering to the subject in need thereof a therapeutically effective amount of a compound of one of embodiments 1 to 75, or a pharmaceutically acceptable salt thereof.

Embodiment 81. A method of treating an N-Ras(G12D)-associated disease in a subject in need thereof, said method comprising administering to the subject in need thereof a therapeutically effective amount of a compound of one of embodiments 1 to 75, or a pharmaceutically acceptable salt thereof.

Embodiment 82. A method of modulating the level of activity of a K-Ras protein in a cell, said method comprising contacting the cell with an effective amount of a compound of one of embodiments 1 to 75, or a pharmaceutically acceptable salt thereof.

Embodiment 83. The method of embodiment 82, wherein said modulating of said activity comprises modulating GTPase activity, nucleotide exchange, differential GDP or GTP binding, effector protein binding, effector protein activation, guanine exchange factor (GEF) binding, GEF-facilitated nucleotide exchange, phosphate release, nucleotide release, nucleotide binding, K-Ras subcellular localization, K-Ras post-translational processing, or K-Ras post-translational modifications.

Embodiment 84. The method of one of embodiments 82 to 83, wherein said modulating is increasing the activity of said K-Ras protein.

Embodiment 85. The method of one of embodiments 82 to 83, wherein said modulating is reducing the activity of said K-Ras protein.

Embodiment 86. The method of one of embodiments 82 to 85, wherein said K-Ras protein is a human K-Ras protein.

Embodiment 87. The method of embodiment 86, wherein said human K-Ras protein contains a G12D mutation.

Embodiment 88. A method of modulating the level of activity of an H-Ras protein in a cell, said method comprising contacting the cell with an effective amount of a compound of one of embodiments 1 to 75, or a pharmaceutically acceptable salt thereof.

Embodiment 89. The method of embodiment 88, wherein said modulating of said activity comprises modulating GTPase activity, nucleotide exchange, differential GDP or GTP binding, effector protein binding, effector protein activation, guanine exchange factor (GEF) binding, GEF-facilitated nucleotide exchange, phosphate release, nucleotide release, nucleotide binding, H-Ras subcellular localization, H-Ras post-translational processing, or H-Ras post-translational modifications.

Embodiment 90. The method of one of embodiments 88 to 89, wherein said modulating is increasing the activity of said H-Ras protein.

Embodiment 91. The method of one of embodiments 88 to 89, wherein said modulating is reducing the activity of said H-Ras protein.

Embodiment 92. The method of one of embodiments 88 to 89, wherein said H-Ras protein is a human H-Ras protein.

Embodiment 93. The method of embodiment 92, wherein said human H-Ras protein contains a G12D mutation.

Embodiment 94. A method of modulating the level of activity of an N-Ras protein in a cell, said method comprising contacting the cell with an effective amount of a compound of one of embodiments 1 to 75, or a pharmaceutically acceptable salt thereof.

Embodiment 95. The method of embodiment 94, wherein said modulating of said activity comprises modulating GTPase activity, nucleotide exchange, differential GDP or GTP binding, effector protein binding, effector protein activation, guanine exchange factor (GEF) binding, GEF-facilitated nucleotide exchange, phosphate release, nucleotide release, nucleotide binding, N-Ras subcellular localization, N-Ras post-translational processing, or N-Ras post-translational modifications.

Embodiment 96. The method of one of embodiments 94 to 95, wherein said modulating is increasing the activity of said N-Ras protein.

Embodiment 97. The method of one of embodiments 94 to 95, wherein said modulating is reducing the activity of said N-Ras protein.

Embodiment 98. The method of one of embodiments 94 to 97, wherein said N-Ras protein is a human K-Ras protein.

Embodiment 99. The method of embodiment 98, wherein said human N-Ras protein contains a G12D mutation.

Embodiment 100. A K-Ras protein covalently bound to a compound of one of embodiments 1 to 75, or a pharmaceutically acceptable salt thereof, wherein said compound is covalently bound to an aspartate residue of said K-Ras protein.

Embodiment 101. The covalently modified K-Ras protein of embodiment 100, wherein said compound is reversibly covalently bound to an aspartate residue of said K-Ras protein.

Embodiment 102. The covalently modified K-Ras protein of embodiment 100, wherein said compound is irreversibly covalently bound to an aspartate residue of said K-Ras protein.

Embodiment 103. The covalently modified K-Ras protein of one of embodiments 100 to 102, wherein said covalently modified K-Ras protein has a modulated activity relative to a control, wherein said activity is selected from GTPase activity, nucleotide exchange, differential GDP or GTP binding, effector protein binding, effector protein activation, guanine exchange factor (GEF) binding, GEF-facilitated nucleotide exchange, phosphate release, nucleotide release, nucleotide binding, K-Ras subcellular localization, K-Ras post-translational processing, and K-Ras post-translational modifications.

Embodiment 104. The covalently modified K-Ras protein of one of embodiments 100 to 103, wherein said K-Ras protein contains a G12D mutation.

Embodiment 105. The covalently modified K-Ras protein of embodiment 104, wherein said compound is covalently bonded to aspartate residue 12.

Embodiment 106. An H-Ras protein covalently bound to a compound of one of embodiments 1 to 75, or a pharmaceutically acceptable salt thereof, wherein said compound is covalently bound to an aspartate residue of said H-Ras protein.

Embodiment 107. The covalently modified H-Ras protein of embodiment 106, wherein said compound is reversibly covalently bound to an aspartate residue of said H-Ras protein.

Embodiment 108. The covalently modified H-Ras protein of embodiment 106, wherein said compound is irreversibly covalently bound to an aspartate residue of said H-Ras protein.

Embodiment 109. The covalently modified H-Ras protein of one of embodiments 106 to 108, wherein said covalently modified H-Ras protein has a modulated activity relative to a control, wherein said activity is selected from GTPase activity, nucleotide exchange, differential GDP or GTP binding, effector protein binding, effector protein activation, guanine exchange factor (GEF) binding, GEF-facilitated nucleotide exchange, phosphate release, nucleotide release, nucleotide binding, H-Ras subcellular localization, H-Ras post-translational processing, and H-Ras post-translational modifications.

Embodiment 110. The covalently modified H-Ras protein of one of embodiments 106 to 109, wherein said H-Ras protein contains a G12D mutation.

Embodiment 111. The covalently modified H-Ras protein of embodiment 110, wherein said compound is covalently bonded to aspartate residue 12.

Embodiment 112. An N-Ras protein covalently bound to a compound of one of embodiments 1 to 75, or a pharmaceutically acceptable salt thereof, wherein said compound is covalently bound to an aspartate residue of said N-Ras protein.

Embodiment 113. The covalently modified N-Ras protein of embodiment 112, wherein said compound is reversibly covalently bound to an aspartate residue of said N-Ras protein.

Embodiment 114. The covalently modified N-Ras protein of embodiment 112, wherein said compound is irreversibly covalently bound to an aspartate residue of said N-Ras protein.

Embodiment 115. The covalently modified N-Ras protein of one of embodiments 112 to 114, wherein said covalently modified H-Ras protein has a modulated activity relative to a control, wherein said activity is selected from GTPase activity, nucleotide exchange, differential GDP or GTP binding, effector protein binding, effector protein activation, guanine exchange factor (GEF) binding, GEF-facilitated nucleotide exchange, phosphate release, nucleotide release, nucleotide binding, N-Ras subcellular localization, N-Ras post-translational processing, and N-Ras post-translational modifications.

Embodiment 116. The covalently modified N-Ras protein of one of embodiments 112 to 115, wherein said H-Ras protein contains a G12D mutation.

Embodiment 117. The covalently modified N-Ras protein of embodiment 116, wherein said compound is covalently bonded to aspartate residue 12.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### EXAMPLES

### Example 1: Chemical Acylation of an Acquired Serine Suppresses Oncogenic Signaling of Ras(G12S)

Mutations in the KRAS protooncogene are the most frequently observed genetic lesion in human cancer and are estimated to account for one million deaths every year worldwide (1, 2). The KRAS gene encodes a small GTPase that controls pro-growth signaling in cells by cycling between the GTP-bound and the GDP-bound states. Hotspot mutations on KRAS compromise GTP hydrolysis (3-5), leading to prolonged and enhanced signaling transduction. Direct, mutant-selective inhibition of oncogenic K-Ras mutants present ideal therapeutic opportunities, which have been pursued with various modalities including small molecules (6, 7, 8-18), cyclic peptides (19-21), therapeutic macromolecules (22-27) and targeted protein degraders (28, 29), etc. (30). The discovery of the allosteric Switch-II pocket (S—IIP) and identification of covalent ligands of K-Ras(G12C) demonstrated that K-Ras is a druggable target, whose inhibition confers marked clinical benefit. To date, one such inhibitor (sotorasib) has received approval by the US FDA, and at least five additional drug candidates (adagrasib (31), JNJ-74699157 (32), LY3499446 (33), GDC6036 (34), and JDQ443 (35)) are under clinical investigation. Despite this success, covalent drugs that target other non-cysteine hotspot mutants of K-Ras remain to be developed due to the low nucleophilicity of residues other than cysteine. The Gly-12 codon in KRAS (GGT) is a site of multiple base changes observed in cancer. The smoking induced transversion mutation (c. 34G>T) to produce the TGT codon gives rise to the druggable G12C oncogene. The non-smoking related transition mutation (c. 35G>A) at the second position produces the GAT codon which is most common G12D allele. The c. 34G>A transition at the first position of produces the serine codon (AGT). Disclosed herein, inter alia, are small molecules that irreversibly bind to K-Ras(G12S), a hot spot mutant accounting for 4.4% of all KRAS mutations (36). In order to covalently target a serine residue we were guided by a family of natural products which possess a strained β-lactone (e.g., salinosporamide A, omuralide, etc.) and inhibit the 20S proteasome by forming a covalent bond with the catalytic threonine (Thr1) (37, 38). It is shown that K-Ras ligands which possess a β-lactone are potent electrophiles that bind to K-Ras(G12S) in the Switch-II Pocket (S—IIP) and rapidly acylate the mutant serine residue, in much the same way that acrylamides are used to target the much more nucleophilic cysteine side chain. Molecules have been identified that fully engage K-Ras(G12S) in cells and suppress its oncogenic signaling without affecting its wildtype counterpart.

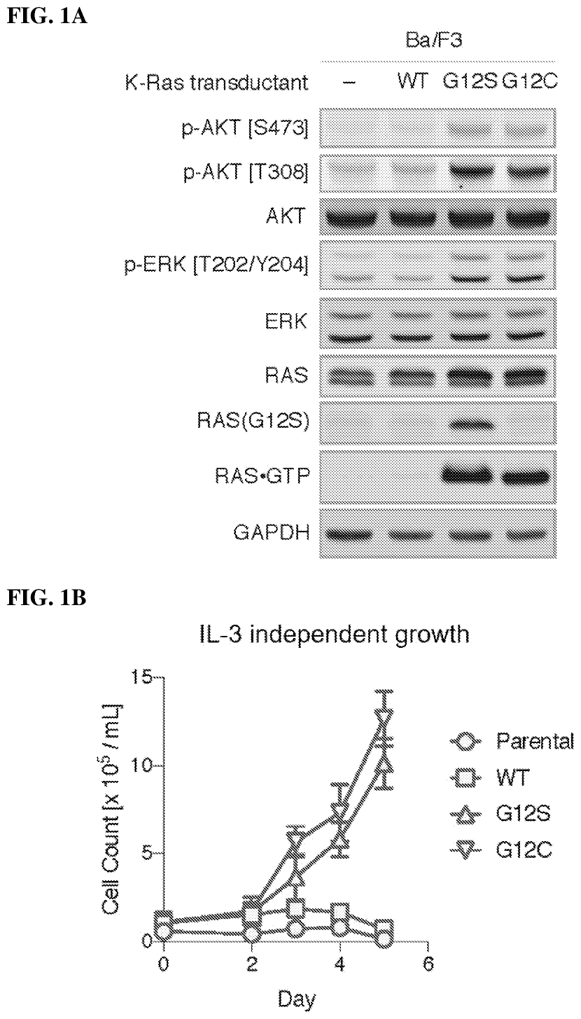

The KRAS p. G12S mutation has been observed in thousands of patient tumors (39), occurring in 2.8% of colorectal adenocarcinoma and 2.5% of non-small cell lung cancer (40). The same mutation on the HRAS gene is reported to be an activating mutation (41) and a prevalent mutation in Costello syndrome (42). We asked whether the glycine-to-serine change at codon 12 alone is sufficient to create a mutant K-Ras protein capable of oncogenic signaling. To assess oncogenic transformation by K-Ras(G12S), we took advantage of the Ba/F3 system, an immortalized murine cell line whose growth depends on exogenous IL-3 but exhibits IL-3-independent growth upon transformation. We generated Ba/F3 variants that stably express wildtype K-Ras or K-Ras(G12S) by infecting the cells with ecotropic retroviruses and verified the ectopic expression of K-Ras by immunoblot using pan-Ras and Ras(G12S)-specific antibodies (FIG. 1B). We also generated a variant expressing K-Ras(G12C), a common hotspot mutant of K-Ras in human cancer, as a comparator. Relative to the parental line, the Ba/F3 cells expressing K-Ras(G12S) and K-Ras(G12C) had elevated phospho-ERK and phospho-AKT levels, whereas those expressing wild-type K-Ras did not (FIG. 1B). Consistent with this observation, the K-Ras(G12S) and K-Ras(G12C) expressing cells continued to proliferate at comparable rates after IL-3 had been removed from the culture medium (FIG. 1C). Together, these results suggest that KRAS G12S is an oncogenic driver mutation with similar transformation potential to KRAS G12C.

We next asked whether the G12S mutation hampers the rate of GTP hydrolysis, a common biochemical mechanism that confers functional activation and extended pro-growth signaling to K-Ras hotspot mutants (5). We measured single turnover GTP hydrolysis by K-Ras(G12S) using a purine nucleoside phosphorylase-coupled assay that monitors free phosphate formation (FIG. 1A). Compared to wild-type K-Ras, K-Ras(G12S) showed a diminished intrinsic GTP hydrolysis rate, and importantly, was insensitive to the GTPase-activating protein NF1-mediated acceleration.

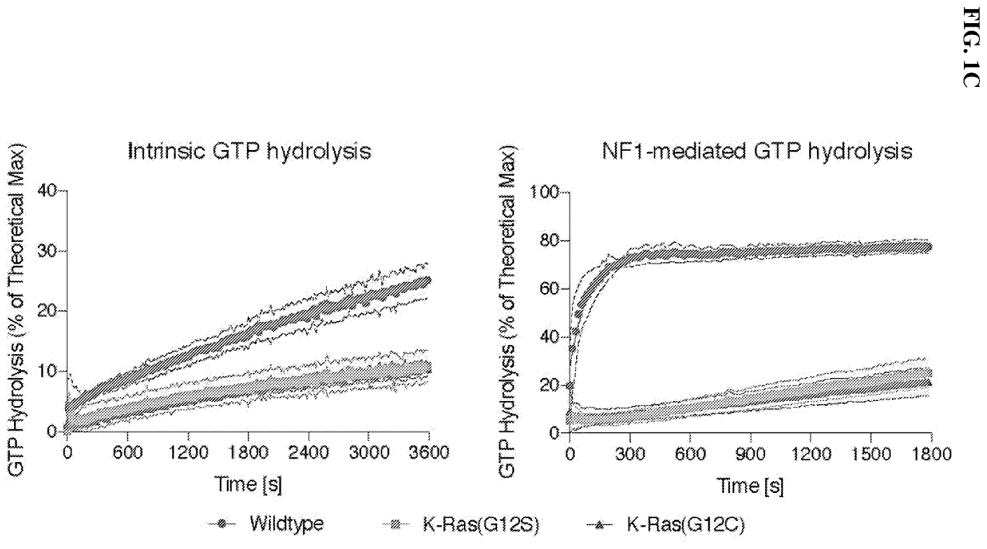

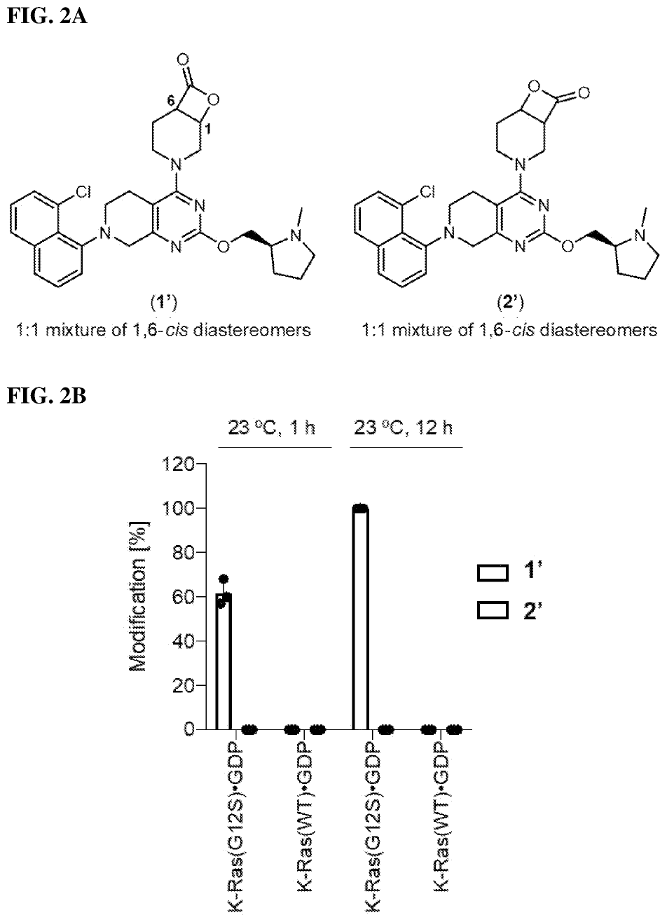

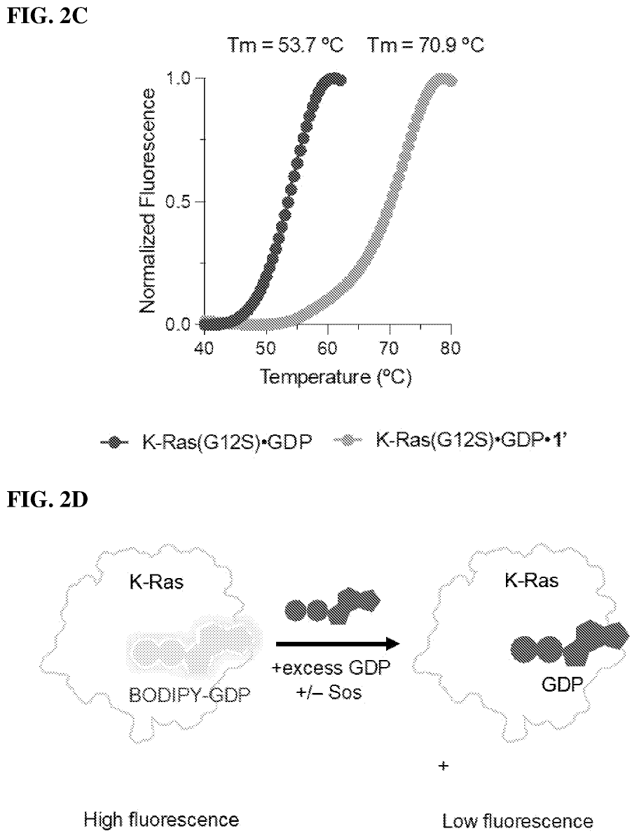

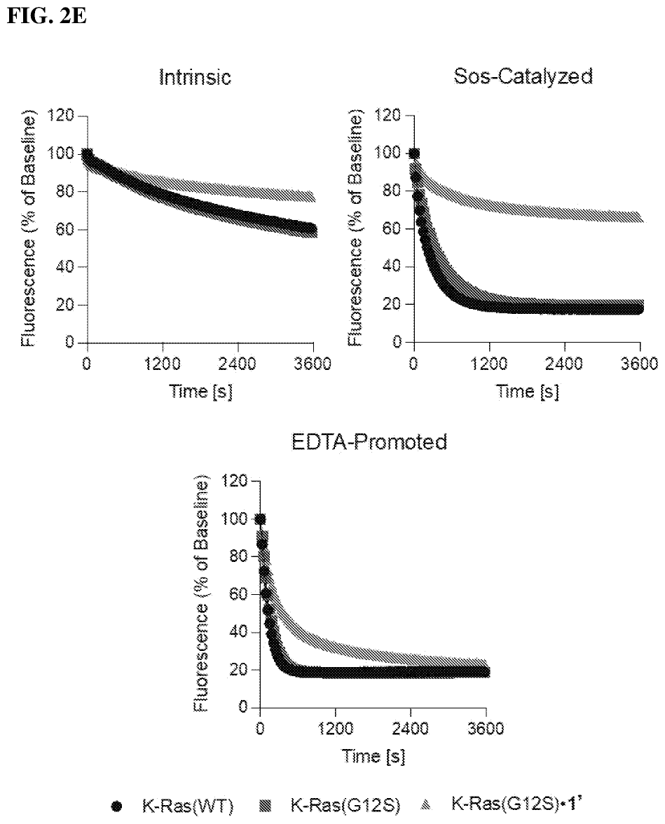

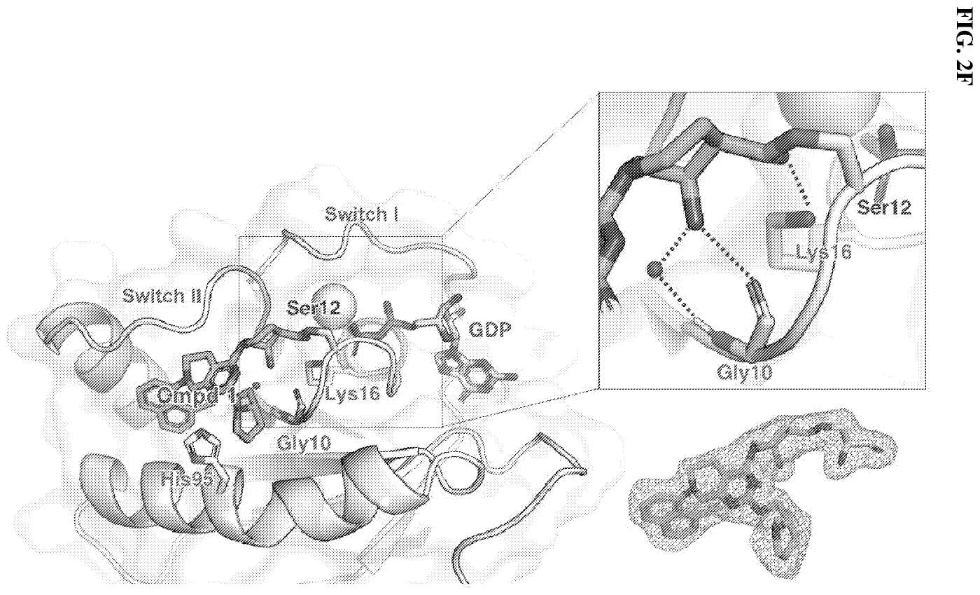

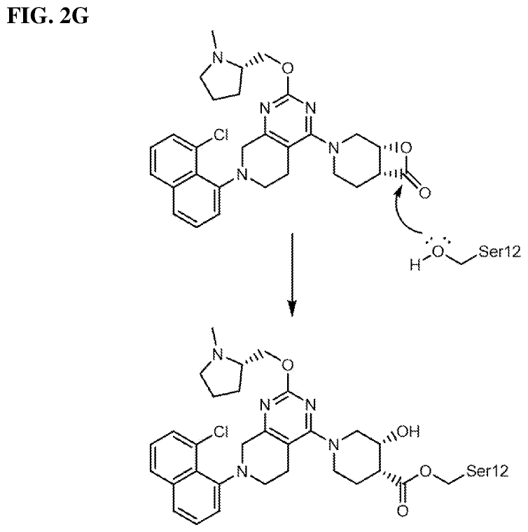

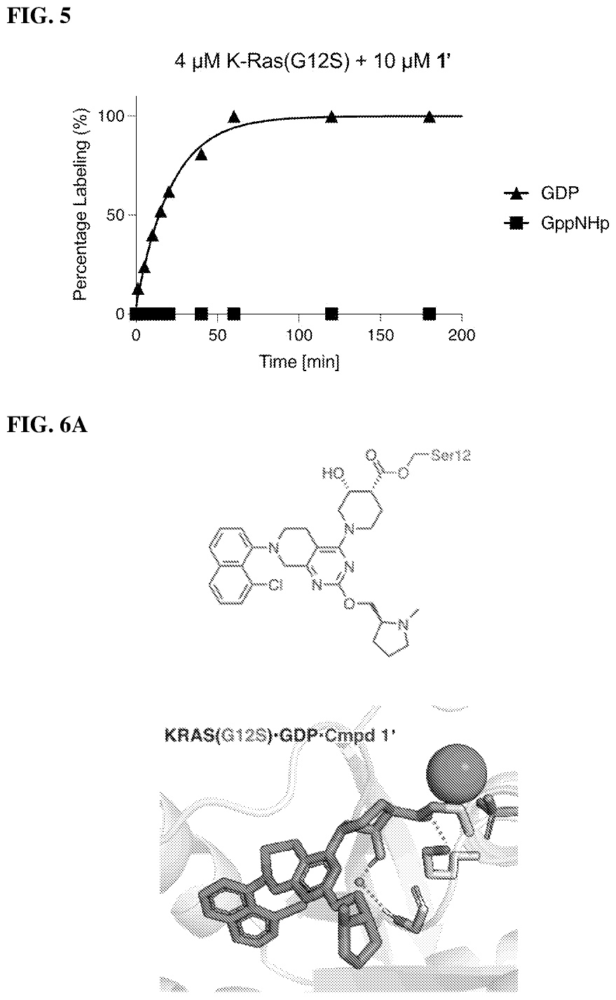

The residual intrinsic hydrolytic activity of GTP suggests that the GDP-bound state may constitute a significant population of cellular K-Ras(G12S) and may be targetable by small molecule ligands, especially those that irreversibly engage the protein through covalent bond formation. To design K-Ras(G12S) targeting compounds, we drew lessons from the successful drug discovery efforts directed against K-Ras(G12C) (11, 16, 18, 43), as well as a family of β-lactone-containing natural products (e.g., salinosporamide A, omuralide, etc.) that inhibit the 20S proteasome by forming a covalent bond with the catalytic threonine (Thr1) (37, 38). We chose to target the S—IIP because it proves to be a privileged drug binding pocket for K-Ras and offers direct access to the mutant residues at codon 12. Compounds 1′ and 2′ (FIG. 2A) were synthesized by attaching a pair of β-lactone electrophiles to the tetrahydropyridopyrimidine moiety found in the clinical candidate MRTX849 and evaluated their ability to covalently engage recombinant K-Ras(G12S) at the mutant serine residue using whole-protein mass spectrometry. Covalent adduct formation was observed between compound 1′ (10 μM) and K-Ras(G12S)·GDP (4 μM), with the extent of modification reaching 64% after 1 hour at 23° C. and 100% after 12 h (FIG. 2B). By contrast, compound 2′, a regioisomer of compound 1′, did not yield any covalent adduct under identical conditions, and neither of these compounds formed covalent adducts with wild-type K-Ras protein. The adduct formation was accompanied with demonstrable thermal stabilization (FIG. 2C), increasing the melting temperature (Tm) of K-Ras(G12S)·GDP from 53.7° C. to 70.9° C. (+17.2° C.). The reaction between 1′ and K-Ras(G12S) is selective for the GDP-bound state; no reaction was observed with GppNHp-loaded K-Ras(G12S) protein under identical conditions (FIG. 5).

To assess whether compound 1′ affects nucleotide cycling of K-Ras, nucleotide exchange experiments were performed by monitoring the exchange of a fluorescent GDP analog (BODIPY-GDP) for unlabeled GDP in the presence of the guanine nucleotide exchange factor Sos or the metal chelator EDTA (FIGS. 2D-2E). As was seen with S—IIP ligands of K-Ras(G12C), compound 1′ blocked Sos-catalyzed exchange and decreased the rate of EDTA promoted exchange.

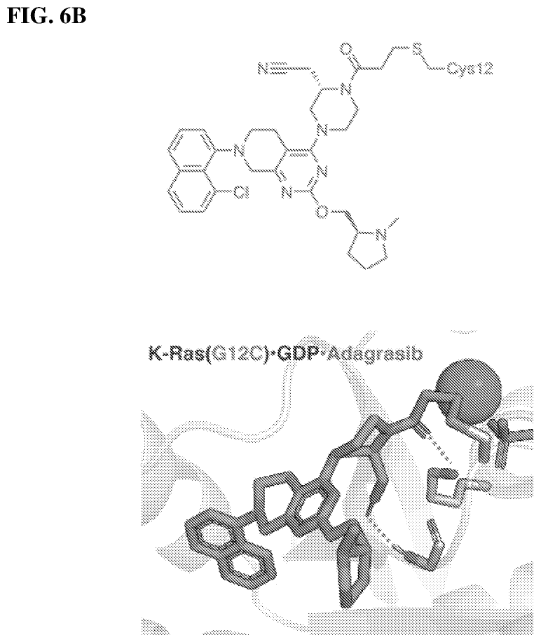

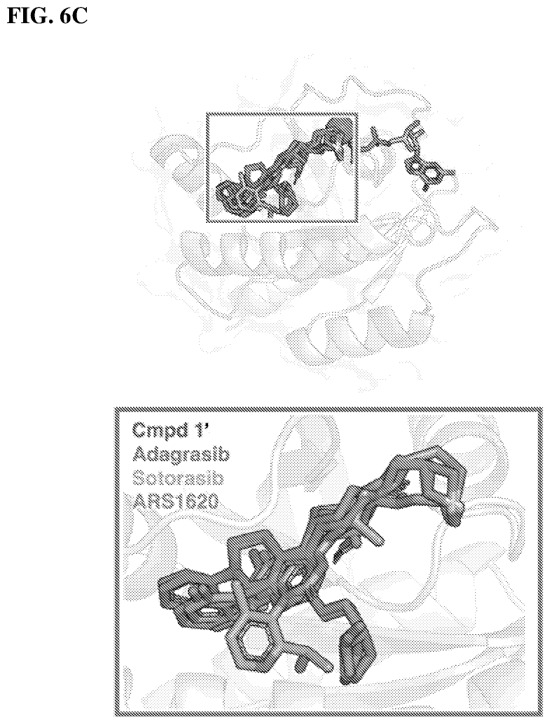

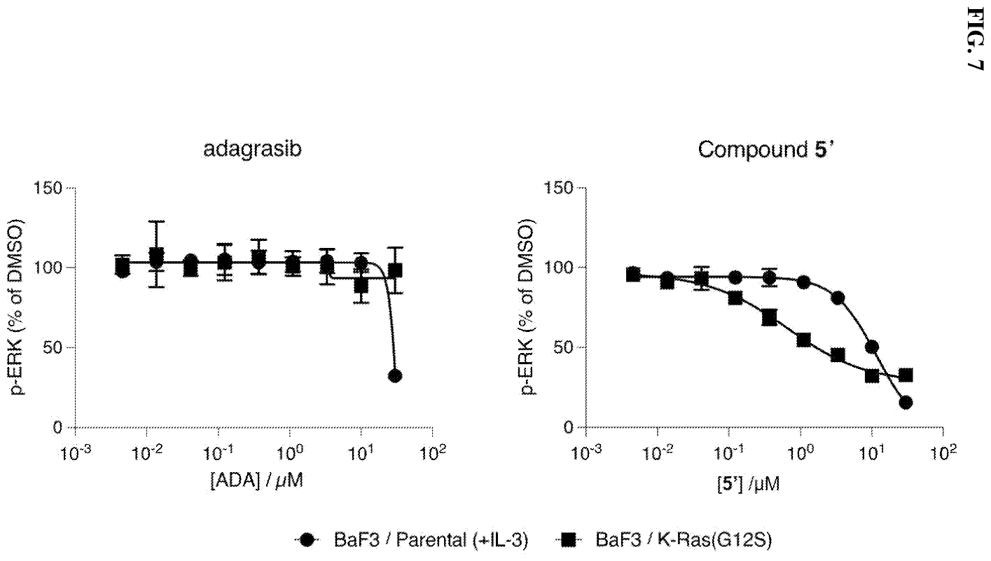

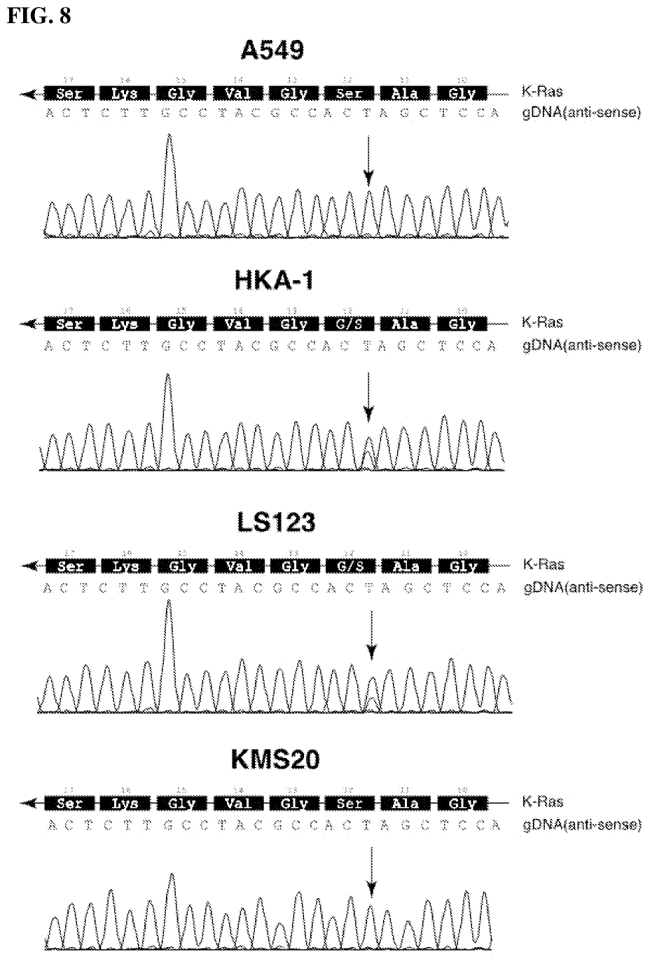

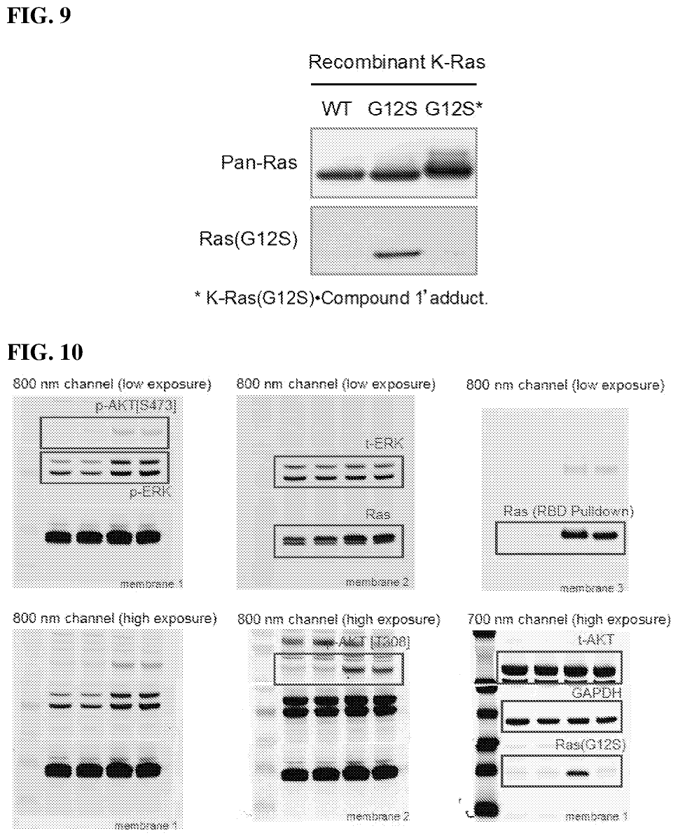

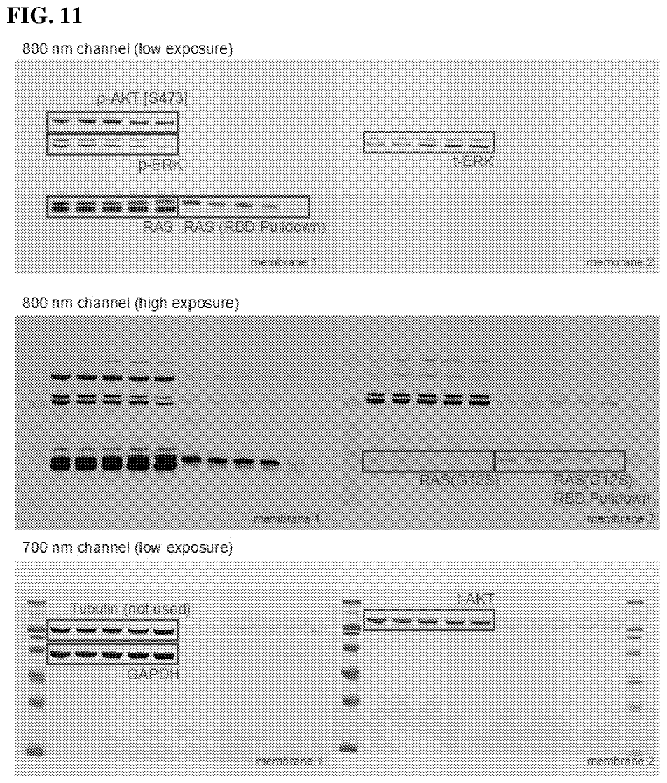

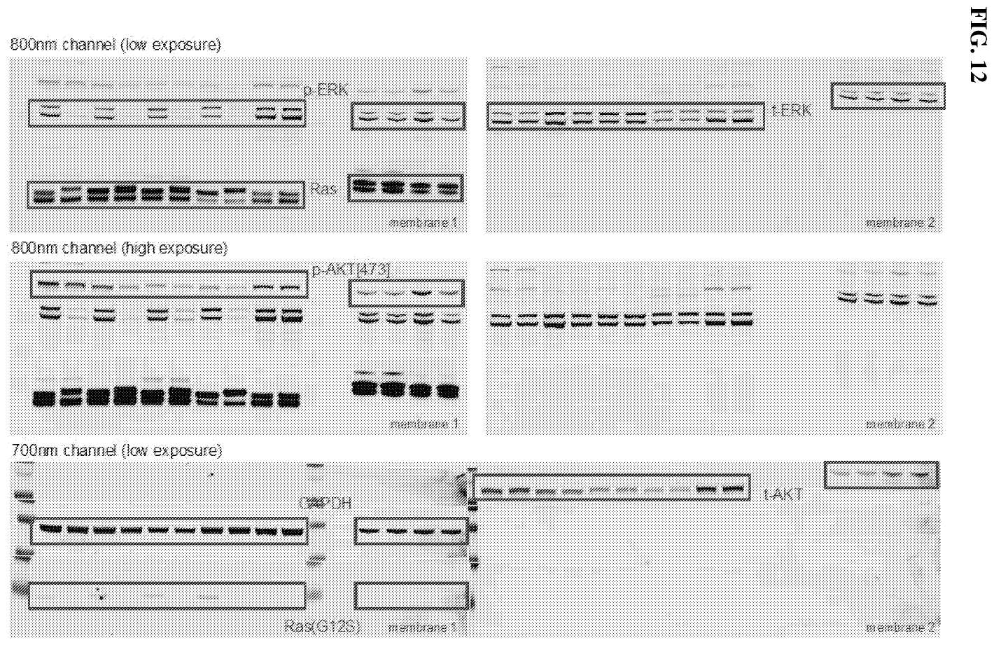

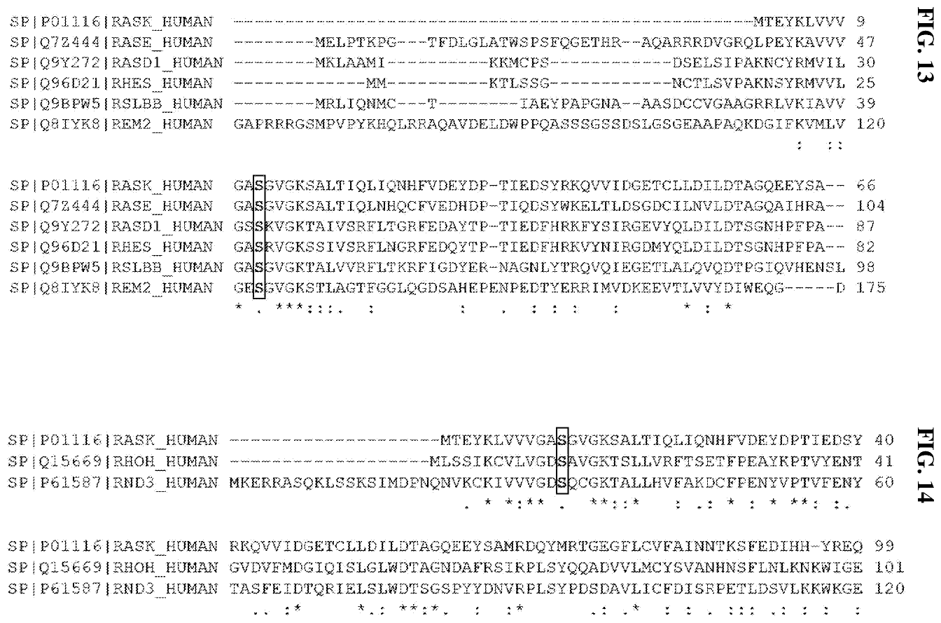

β-Lactones are highly strained electrophiles with an estimated strain energy of 22.7 kcal/mol (44). We hypothesized that the adduct formation between K-Ras(G12S)·GDP and compound 1′ involved a nucleophilic attack from the mutant serine residue to the carbonyl group of the β-lactone in 1′ and subsequent ring opening (FIG. 2G). To test this hypothesis, we solved a 2.0-Å crystal structure of the K-Ras(G12S)·GDP·1′ complex (FIG. 2F). Comparison of this structure with previously reported co-crystal structures of S—IIP ligands with K-Ras(G12C) shows that compound 1′ binds in the S—IIP and adopts an orientation similar to the G12C ligands (RMSD 0.273 Å, FIGS. 6A-6C). Well-defined electron density confirmed that Ser12 was acylated by compound 1′ (FIG. 2F inset), giving rise to a protein-drug complex mediated by an ester linkage in which the strained β-lactone ester is replaced by an unstrained ester. We also observed that the carbonyl oxygen of this ester group is engaged in a hydrogen bond with Lys16, and that the secondary alcohol resulting from the β-lactone opening formed a hydrogen bond with the backbone carbonyl of glycine 10 (FIG. 2F inset). A water molecule was also observed bridging the same secondary alcohol and the backbone N—H of glycine 10. Hydrogen bonding between Lys16 and the carbonyl group on acrylamide electrophiles has been proposed to great enhance the reactivity for K-Ras(G12C) inhibitors (45). These anchoring interactions explain the drastic difference in activity between the closely related compounds 1′ and 2′, as the latter not only has a misaligned electrophile but also lacks the correct geometry to form either of these hydrogen bonds.

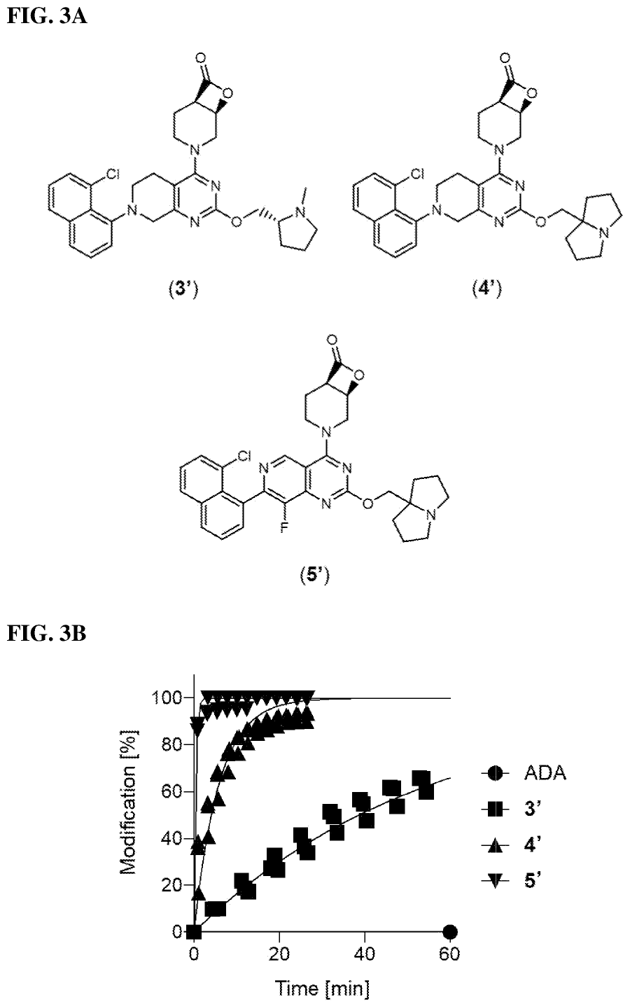

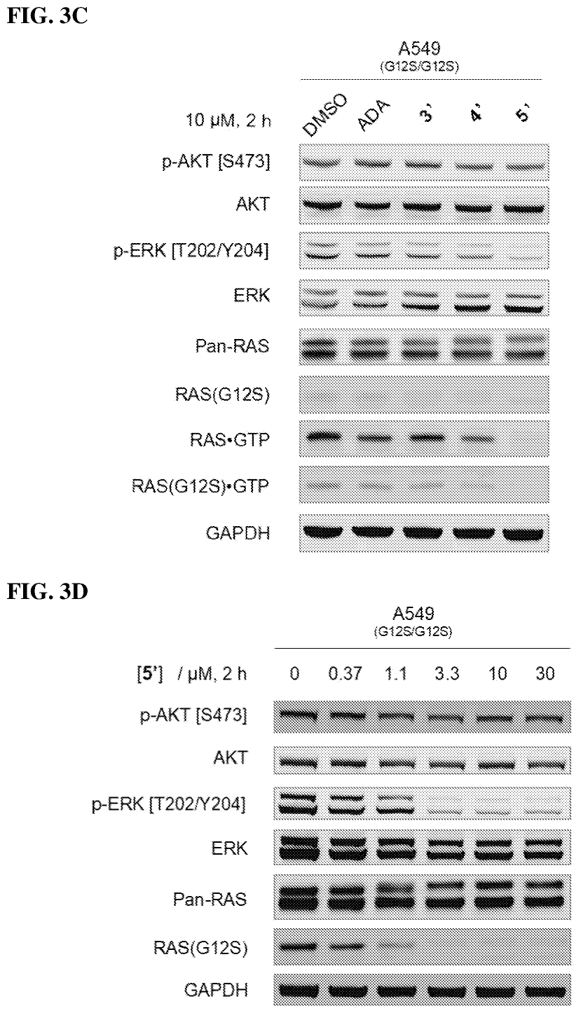

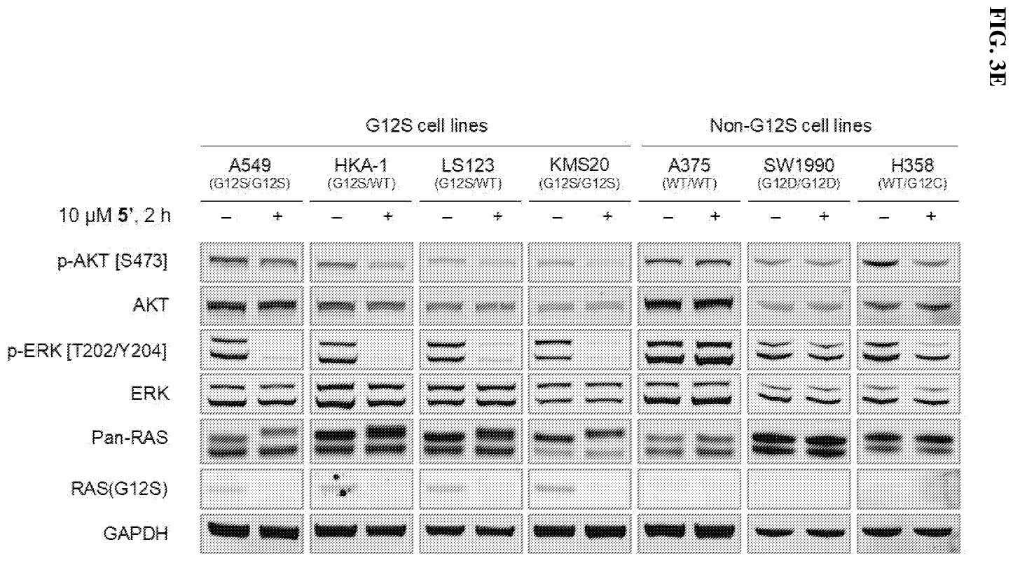

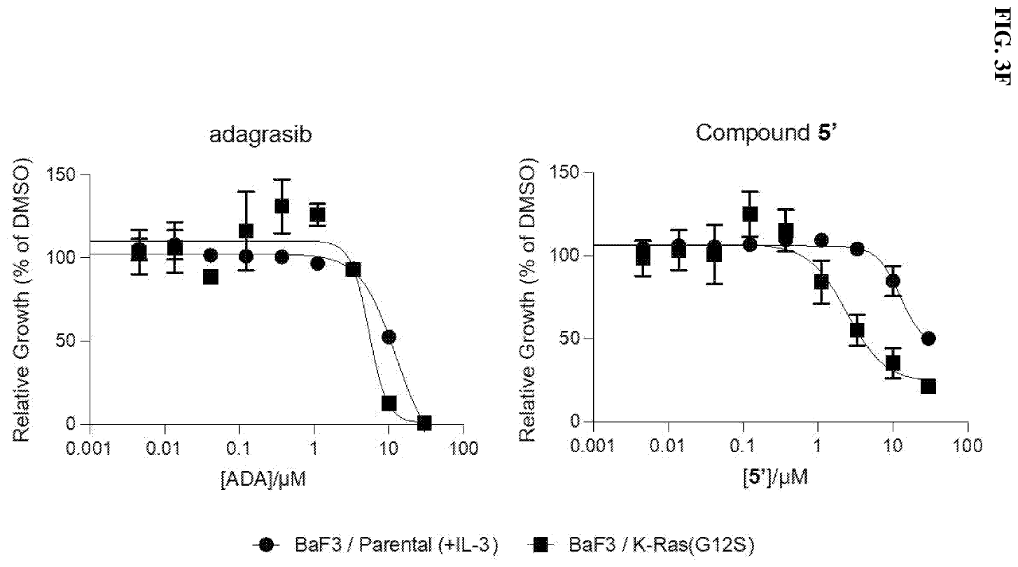

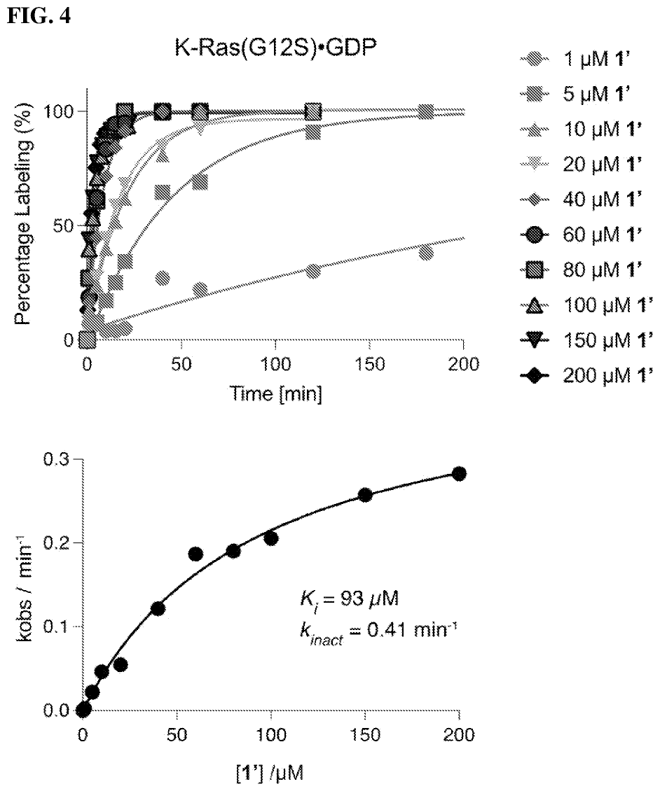

This structural analysis also reaffirmed the β-lactone, being part of a [4.2.0] bicyclic system, as the core pharmacophore in 1′. Although compound 1′ was prepared as a mixture of diastereomers, it is evident that (1R, 6R)-1′ (depicted in FIG. 2G) was the dominantly active diastereomer. As we sought to improve the potency of 1′, we first synthesized the (1R, 6R)-1′ with high diastereomeric purity (hereafter referred to as 3′) using the intramolecular nucleophile-catalyzed aldol lactonization (NCAL) method (see Example 2) (46, 47). Because the reversible inhibitory constant Kᵢ (97 μM) and the first order rate constant k_{inact} (0.41 min⁻¹) we measured for 1′ (FIG. 4) suggested potential benefits of improving the reversible binding, we also varied the N-methylprolinol substituent and the tetrahydropyridopyrimidine moiety based on recent patent literature (48), yielding compounds 4′ and 5′ (FIG. 3A). Compounds 3′, 4′ and 5′ all underwent enhanced reaction with recombinant K-Ras(G12S)·GDP protein, with 5′ being the most potent, reaching 100% modification in <10 min at 10 μM (FIG. 3B). By comparison, the K-Ras(G12C)-targeting clinical candidate adagrasib, bearing a 2-fluoroacrylamide electrophile, did not react with K-Ras(G12S)·GDP even after extended incubation.

We asked whether these optimized β-lactones allows targeted inhibition of K-Ras(G12S) in genetically characterized cancer cell lines. We treated A549 cells (homozygous KRAS p.G12S mutation) with 10 μM adagrasib, 3′, 4′, or 5′ for 2 h and monitored phospho-Akt and phospho-Erk levels. We also took advantage of a mutant selective antibody which recognizes Ras(G12S) that does not react with covalently modified K-Ras(G12S) protein (see Example 2 for its validation) and used it in concert with Raf-RBD pulldown to assess the intracellular level of GTP-bound K-Ras(G12S). While neither adagrasib nor 3′ had any observable effects on these markers, treatment by 4′ decreased the level of Ras·GTP, and treatment by 5′ led to a nearly complete loss of Ras·GTP and concomitant inhibition of phospho-Erk (FIG. 3C). These changes were accompanied by direct adduct formation with cellular K-Ras(G12S), as seen in the upward shift of the K-Ras band in the anti-Ras blot and the disappearance of the signal in the anti-Ras(G12S) blot. The inhibition of Ras signaling by 5′ in A549 cells was dose-dependent, with an apparent IC₅₀ of about 3 μM under these treatment conditions (FIG. 3E). We next examined four cell lines with confirmed KRAS p.G12S mutations (see Example 2 for sequencing data). In each of these cell lines, treatment with compound 5′ led to a reduction of phospho-ERK level and gel mobility shift of the K-Ras protein (FIG. 3D). Meanwhile, compound 5′ did not perturb the signaling in A375 cells (wild-type KRAS) or SW1990 cells (homozygous KRAS p.G12D), and only had a weak inhibition of phospho-Erk in H358 cells (heterozygous KRAS p.G12C), an effect we ascribed to the possible ring opening of the β-lactone by the nucleophilic cysteine.

While these data confirmed the specificity of 5′ against the KRAS G12S allele, we reasoned that the chemoselectivity encoded by the β-lactone group could afford a therapeutic window between cells expressing the mutant K-Ras(G12S) and those expressing wild-type K-Ras. To test this, we compared the effects of 5′ on the proliferation of Ba/F3:K-Ras(G12S) cells (without IL-3) and the parental Ba/F3 cells (with IL-3). Compound 5′ preferentially inhibited the growth of the K-Ras(G12S)-transduced cells with an IC₅₀ of 2.4 μM and, to a lesser extent, that of the parental cells with an IC₅₀ of 12.5 μM. Notably, the non-G12S targeting compound adagrasib did not display such selectivity and caused complete cell death at 10 μM for both cell lines. Consistent with this result, treatment of Ba/F3:K-Ras(G12S) cells with compound 5′ led to a dose-dependent reduction of phospho-ERK levels (FIG. 7). Although the five-fold selectivity observed with compound 5′ merits extensive optimization, the data herein provides the first example of selective targeting of the K-Ras(G12S) mutant using small molecule agents.

The breakthrough discovery that the mutant cysteine in K-Ras(G12C) can be exploited by small molecule electrophiles has fueled renewed efforts to develop K-Ras targeting agents, culminating in the recent FDA approval of sotorasib with five additional drug candidates under clinical investigation. Nevertheless, therapeutic strategies for other hotspot mutants of K-Ras have not been reported. Compared to cysteine, the acquired serine residue in K-Ras(G12S) has much weaker nucleophilicity and does not react with electrophiles tailored for K-Ras(G12C). Inspired by a family of threonine targeting natural products containing the four membered strained β-lactone we sought to target the G12S allele of K-Ras. Using structure-guided chemical design, we identified β-lactone as a privileged electrophile for the acquired serine in K-Ras(G12S) and synthesized S—IIP ligands that rapidly undergo covalent engagement with this mutant residue. Similar to the case of K-Ras(G12C) inhibitors, even though the compounds in Example 1 do not bind to the GTP-bound form of K-Ras(G12S), its intrinsic GTPase activity is sufficient to support the complete irreversible trapping of this mutant allele and allele-specific suppression of oncogenic cellular signaling.

Natural (49) and synthetic β-lactones are known to undergo ring opening following nucleophilic attack from catalytically active serine or threonine residues in enzymes (e.g., omuralide/20S proteasome, lipstatin/pancreatic lipase, palmostatin B/acyl protein thioesterase, etc.). Yet, to the best of our knowledge, such reactivity has not been observed with non-catalytic serines, including the acquired serines in mutant proteins. Our work demonstrates that chemical acylation of a non-catalytic serine can be achieved using β-lactone electrophiles (50, 51).

### REFERENCES FOR EXAMPLE 1

1. Simanshu, D. K.; Nissley, D. V.; McCormick, F. Cell 2017, 170 (1), 17-33. 2. Prior, I. A.; Hood, F. E.; Hartley, J. L. Cancer Res. 2020, 80 (14), 2969-2974. 3. Gibbs, J. B.; Sigal, I. S.; Poe, M.; Scolnick, E. M. Proc. Natl. Acad. Sci. U.S.A. 1984, 81 (18 I), 5704-5708. 4. Trahey, M.; Mccormick, F. Science 1987, 238 (4826), 542-545. 5. Hunter, J. C.; Manandhar, A.; Carrasco, M. A.; Gurbani, D.; Gondi, S.; Westover, K. D. Mol. Cancer Res. 2015, 1325-1336. 6. Taveras, A. G.; Remiszewski, S. W.; Doll, R. J.; Cesarz, D.; Huang, E. C.; Kirschmeier, P.; Pramanik, B. N.; Snow, M. E.; Wang, Y. S.; Del Rosario, J. D.; Vibulbhan, B.; Bauer, B. B.; Brown, J. E.; Carr, D.; Catino, J.; Evans, C. A.; Girijavallabhan, V.; Heimark, L.; James, L.; Liberles, S.; Nash, C.; Perkins, L.; Senior, M. M.; Tsarbopoulos, A.; Ganguly, A. K.; Aust, R.; Brown, E.; Delisle, D.; Fuhrman, S.; Hendrickson, T.; Kissinger, C.; Love, R.; Sisson, W.; Villafranca, E.; Webber, S. E. Bioorganic Med. Chem. 1997, 5 (1), 125-133. 7. Patgiri, A.; Yadav, K. K.; Arora, P. S.; Bar-Sagi, D. Nat. Chem. Biol. 2011, 7 (9), 585-587. 8. Maurer, T.; Garrenton, L. S.; Oh, A.; Pitts, K.; Anderson, D. J.; Skelton, N. J.; Fauber, B. P.; Pan, B.; Malek, S.; Stokoe, D.; Ludlam, M. J. C.; Bowman, K. K.; Wu, J.; Giannetti, A. M.; Starovasnik, M. A.; Mellman, I.; Jackson, P. K.; Rudolph, J.; Wang, W.; Fang, G. Proc. Natl. Acad. Sci. U.S.A. 2012, 109 (14), 5299-5304. 9. Sun, Q.; Burke, J. P.; Phan, J.; Burns, M. C.; Olejniczak, E. T.; Waterson, A. G.; Lee, T.; Rossanese, O. W.; Fesik, S. W. Angew. Chem. Int. Ed. 2012, 51 (25), 6140-6143. 10. Shima, F.; Yoshikawa, Y.; Ye, M.; Araki, M.; Matsumoto, S.; Liao, J.; Hu, L.; Sugimoto, T.; Ijiri, Y.; Takeda, A.; Nishiyama, Y.; Sato, C.; Muraoka, S.; Tamura, A.; Osoda, T.; Tsuda, K.-i.; Miyakawa, T.; Fukunishi, H.; Shimada, J.; Kumasaka, T.; Yamamoto, M.; Kataoka, T. Proc. Natl. Acad. Sci. 2013, 110 (20), 8182-8187. 11. Ostrem, J. M.; Peters, U.; Sos, M. L.; Wells, J. A.; Shokat, K. M. K-Ras(G12C) Nature 2013, 503 (7477), 548-551. 12. Lim, S. M.; Westover, K. D.; Ficarro, S. B.; Harrison, R. A.; Choi, H. G.; Pacold, M. E.; Carrasco, M.; Hunter, J.; Kim, N. D.; Xie, T.; Sim, T.; Janne, P. A.; Meyerson, M.; Marto, J. A.; Engen, J. R.; Gray, N. S. Angew. Chem. Int. Ed. 2014, 53 (1), 199-204. 13. Spencer-Smith, R.; Koide, A.; Zhou, Y.; Eguchi, R. R.; Sha, F.; Gajwani, P.; Santana, D.; Gupta, A.; Jacobs, M.; Herrero-Garcia, E.; Cobbert, J.; Lavoie, H.; Smith, M.; Rajakulendran, T.; Dowdell, E.; Okur, M. N.; Dementieva, I.; Sicheri, F.; Therrien, M.; Hancock, J. F.; Ikura, M.; Koide, S.; O'Bryan, J. P. Nat. Chem. Biol. 2017, 13 (1), 62-68. 14. Martin-Gago, P.; Fansa, E. K.; Klein, C. H.; Murarka, S.; Janning, P.; Schurmann, M.; Metz, M.; Ismail, S.; Schultz-Fademrecht, C.; Baumann, M.; Bastiaens, P. I. H.; Wittinghofer, A.; Waldmann, H. Angew. Chem. Int. Ed. 2017, 56 (9), 2423-2428. 15. Welsch, M. E.; Kaplan, A.; Chambers, J. M.; Stokes, M. E.; Bos, P. H.; Zask, A.; Zhang, Y.; Sanchez-Martin, M.; Badgley, M. A.; Huang, C. S.; Tran, T. H.; Akkiraju, H.; Brown, L. M.; Nandakumar, R.; Cremers, S.; Yang, W. S.; Tong, L.; Olive, K. P.; Ferrando, A.; Stockwell, B. R. Cell 2017, 168 (5), 878-889.e29. 16. Janes, M. R.; Zhang, J.; Li, L. S.; Hansen, R.; Peters, U.; Guo, X.; Chen, Y.; Babbar, A.; Firdaus, S. J.; Darjania, L.; Feng, J.; Chen, J. H.; Li, S.; Li, S.; Long, Y. O.; Thach, C.; Liu, Y.; Zarieh, A.; Ely, T.; Kucharski, J. M.; Kessler, L. V.; Wu, T.; Yu, K.; Wang, Y.; Yao, Y.; Deng, X.; Zarrinkar, P. P.; Brehmer, D.; Dhanak, D.; Lorenzi, M. V.; Hu-Lowe, D.; Patricelli, M. P.; Ren, P.; Liu, Y. Cell 2018, 172 (3), 578-589.e17. 17. Canon, J.; Rex, K.; Saiki, A. Y.; Mohr, C.; Cooke, K.; Bagal, D.; Gaida, K.; Holt, T.; Knutson, C. G.; Koppada, N.; Lanman, B. A.; Werner, J.; Rapaport, A. S.; San Miguel, T.; Ortiz, R.; Osgood, T.; Sun, J. R.; Zhu, X.; McCarter, J. D.; Volak, L. P.; Houk, B. E.; Fakih, M. G.; O'Neil, B. H.; Price, T. J.; Falchook, G. S.; Desai, J.; Kuo, J.; Govindan, R.; Hong, D. S.; Ouyang, W.; Henary, H.; Arvedson, T.; Cee, V. J.; Lipford, J. R. Nature 2019, 575 (7781), 217-223. 18. Fell, J. B.; Fischer, J. P.; Baer, B. R.; Blake, J. F.; Bouhana, K.; Briere, D. M.; Brown, K. D.; Burgess, L. E.; Burns, A. C.; Burkard, M. R.; Chiang, H.; Chicarelli, M. J.; Cook, A. W.; Gaudino, J. J.; Hallin, J.; Hanson, L.; Hartley, D. P.; Hicken, E. J.; Hingorani, G. P.; Hinklin, R. J.; Mejia, M. J.; Olson, P.; Otten, J. N.; Rhodes, S. P.; Rodriguez, M. E.; Savechenkov, P.; Smith, D. J.; Sudhakar, N.; Sullivan, F. X.; Tang, T. P.; Vigers, G. P.; Wollenberg, L.; Christensen, J. G.; Marx, M. A. J. Med. Chem. 2020, 63 (13), 6679-6693. 19. Trinh, T. B.; Upadhyaya, P.; Qian, Z.; Pei, D. ACS Comb. Sci. 2016, 18 (1), 75-85. 20. Sakamoto, K.; Kamada, Y.; Sameshima, T.; Yaguchi, M.; Niida, A.; Sasaki, S.; Miwa, M.; Ohkubo, S.; Sakamoto, J. ichi; Kamaura, M.; Cho, N.; Tani, A. Biochem. Biophys. Res. Commun. 2017, 484 (3), 605-611. 21. Zhang, Z.; Gao, R.; Hu, Q.; Peacock, H.; Peacock, D. M.; Dai, S.; Shokat, K. M.; Suga, H. ACS Cent Sci. 2020, 6 (10) 1753-1761. 22. Hons, M.; Niebel, B.; Karnowski, N.; Weiche, B.; Famulok, M. ChemBioChem 2012, 13 (10), 1433-1437. 23. Guillard, S.; Kolasinska-Zwierz, P.; Debreczeni, J.; Breed, J.; Zhang, J.; Bery, N.; Marwood, R.; Tart, J.; Overman, R.; Stocki, P.; Mistry, B.; Phillips, C.; Rabbitts, T.; Jackson, R.; Minter, R. Nat. Commun. 2017, 8 (1), 16111. 24. Cetin, M.; Evenson, W. E.; Gross, G. G.; Jalali-Yazdi, F.; Krieger, D.; Arnold, D.; Takahashi, T. T.; Roberts, R. W. J. Mol. Biol. 2017, 429 (4), 562-573. 25. Kauke, M. J.; Traxlmayr, M. W.; Parker, J. A.; Kiefer, J. D.; Knihtila, R.; McGee, J.; Verdine, G.; Mattos, C.; Dane Wittrup, K. Sci. Rep. 2017, 7 (1), 1-9. 26. McGee, J. H.; Shim, S. Y.; Lee, S. J.; Swanson, P. K.; Jiang, S. Y.; Durney, M. A.; Verdine, G. L. J. Biol. Chem. 2018, 293 (9), 3265-3280. 27. Teng, K. W.; Tsai, S. T.; Hattori, T.; Fedele, C.; Koide, A.; Yang, C.; Hou, X.; Zhang, Y.; Neel, B. G.; O'Bryan, J. P.; Koide, S. Nat. Commun. 2021, 12 (1), 1-13. 28. Zeng, M.; Xiong, Y.; Safaee, N.; Nowak, R. P.; Donovan, K. A.; Yuan, C. J.; Nabet, B.; Gero, T. W.; Fern, F.; Li, L.; Gondi, S.; Ombelets, L. J.; Quan, C.; Janne, P. A.; Kostic, M.; Scott, D. A.; Westover, K. D.; Fischer, E. S.; Gray, N. S. Cell Chem. Biol. 2020, 27 (1), 19-31.e6. 29. Bond, M. J.; Chu, L.; Nalawansha, D. A.; Li, K.; Crews, C. ChemRxiv 2020, https://doi.org/10.26434/chemrxiv.12091176.v1. 30. Reck, M.; Carbone, D. P.; Garassino, M.; Barlesi, F. Ann. Oncol. 2021, 32 (9), 1101-1110. 31. Papadopoulos, K. P.; Ou, S.-H. I.; Johnson, M. L.; Christensen, J.; Velastegui, K.; Potvin, D.; Faltaos, D.; Chao, R. C. J. Clin. Oncol. 2019, 37 (15_suppl), TPS3161-TPS3161. 32. First-in-Human Study of JNJ-74699157 in Participants With Tumors Harboring the KRAS G12C Mutation https://clinicaltrials.gov/ct2/show/NCT04006301 (accessed Aug. 5, 2021). 33. A Study of LY3499446 in Participants With Advanced Solid Tumors With KRAS G12C Mutation https://clinicaltrials.gov/ct2/show/NCT04165031 (accessed Aug. 5, 2021). 34. A Study to Evaluate the Safety, Pharmacokinetics, and Activity of GDC-6036 Alone or in Combination in Participants With Advanced or Metastatic Solid Tumors With a KRAS G12C Mutation https://clinicaltrials.gov/ct2/show/NCT04449874 (accessed Aug. 5, 2021). 35. Study of JDQ443 in Patients With Advanced Solid Tumors Harboring the KRAS G12C Mutation https://clinicaltrials.gov/ct2/show/NCT04699188 (accessed Aug. 5, 2021). 36. O'Bryan, J. P. Pharmacol. Res. 2019, 139 (October 2018), 503-511. 37. Feling, R. H.; Buchanan, G. O.; Mincer, T. J.; Kauffman, C. A.; Jensen, P. R.; Fenical, W. Angew. Chem. Int. Ed. Engl. 2003, 42 (3), 355-357. 38. Groll, M.; Balskus, E. P.; Jacobsen, E. N. J. Am. Chem. Soc. 2008, 130 (45), 14981-14983. 39. Catalogue of Somatic Mutations in Cancer (COSMIC) Version 94. https:/cancer.sanger.ac.uk/cosmic/ (accessed Aug. 4, 2021). 40. Sweeney, S. M.; Cerami, E.; Baras, A.; Pugh, T. J.; Schultz, N.; Stricker, T.; Lindsay, J.; Del Vecchio Fitz, C.; Kumari, P.; Micheel, C.; Shaw, K.; Gao, J.; Moore, N.; Stricker, T.; Kandoth, C.; Reardon, B.; Lepisto, E.; Gardos, S.; Dang, K.; Guinney, J.; Omberg, L.; Yu, T.; Gross, B.; Heins, Z.; Hyman, D.; Rollins, B.; Sawyers, C.; Solit, D.; Schrag, D.; Velculescu, V.; Andre, F.; Bedard, P.; Levy, M.; Meijer, G.; Rollins, B.; Shaw, K. Cancer Discov. 2017, 7 (8), 818-831. 41. Seeburg, P. H.; Colby, W. W.; Capon, D. J.; Goeddel, D. V.; Levinson, A. D. Nature 2009, 312 (5989), 71-75. 42. Wey, M.; Lee, J.; Jeong, S. S.; Kim, J.; Heo, J. Biochemistry 2013, 52 (47), 8465-8479. 43. Lanman, B. A.; Allen, J. R.; Allen, J. G.; Amegadzie, A. K.; Ashton, K. S.; Booker, S. K.; Chen, J. J.; Chen, N.; Frohn, M. J.; Goodman, G.; Kopecky, D. J.; Liu, L.; Lopez, P.; Low, J. D.; Ma, V.; Minatti, A. E.; Nguyen, T. T.; Nishimura, N.; Pickrell, A. J.; Reed, A. B.; Shin, Y.; Siegmund, A. C.; Tamayo, N. A.; Tegley, C. M.; Walton, M. C.; Wang, H. L.; Wurz, R. P.; Xue, M.; Yang, K. C.; Achanta, P.; Bartberger, M. D.; Canon, J.; Hollis, L. S.; McCarter, J. D.; Mohr, C.; Rex, K.; Saiki, A. Y.; San Miguel, T.; Volak, L. P.; Wang, K. H.; Whittington, D. A.; Zech, S. G.; Lipford, J. R.; Cee, V. J. J. Med. Chem. 2020, 63 (1), 52-65. 44. Bach, R. D.; Dmitrenko, O. J. Am. Chem. Soc. 2006, 128 (14), 4598-4611. 45. Hansen, R.; Peters, U.; Babbar, A.; Chen, Y.; Feng, J.; Janes, M. R.; Li, L. S.; Ren, P.; Liu, Y.; Zarrinkar, P. P. Nat. Struct. Mol. Biol. 2018, 25 (6), 454-462. 46. Cortez, G. S.; Tennyson, R. L.; Romo, D. Journal of the American Chemical Society. 2001, pp 7945-7946. 47. Sikriwal, D.; Dikshit, D. K. Tetrahedron 2011, 67 (1), 210-215. 48. KRAS G12C Inhibitors. United States Patent Application US2020/0331911A1, 2020. 49. Robinson, S. L.; Christenson, J. K.; Wackett, L. P. Nat. Prod. Rep. 2019, 36 (3), 458-475. 50. Shannon, D. A.; Weerapana, E. Curr. Opin. Chem. Biol. 2015, 24, 18-26. 51. Martin, J. S.; MacKenzie, C. J.; Fletcher, D.; Gilbert, I. H. Bioorganic Med. Chem. 2019, 27 (10), 2066-2074.

### Example 2: Experimental Methods

### Cell Culture

Ba/F3 cells were a gift from Dr. Trevor Bivona (UCSF) and were maintained in RPMI 1640 (Gibco 11875093) supplemented with 10% heat-inactivated fetal bovine serum (Axenia Biologix) and 10 ng/mL recombinant mouse interleukin-3 (Gibco PMC0031). A549 cells were obtained from UCSF Cell Culture Facility and maintained in high-glucose (4.5 g/L) DMEM (Gibco 11995073) supplemented with 4 mM L-glutamine, 1 mM sodium pyruvate, and 10% heat-inactivated fetal bovine serum (Axenia Biologix). LS123 cells were obtained from ATCC (CCL-255) and maintained in high-glucose (4.5 g/L) DMEM (Gibco 11995073) supplemented with 4 mM L-glutamine, 1 mM sodium pyruvate, and 10% heat-inactivated fetal bovine serum (Axenia Biologix). HKA-1 cells were obtained from JRCB Cell Bank (JRCB1017) and maintained in high-glucose (4.5 g/L) DMEM (Gibco 11995073) supplemented with 4 mM L-glutamine, 1 mM sodium pyruvate, and 10% heat-inactivated fetal bovine serum (Axenia Biologix). KMS-20 cells were obtained from JRCB Cell Bank (JRCB1196) and maintained in RPMI 1640 (Gibco 11875093) supplemented with 10% heat-inactivated fetal bovine serum (Axenia Biologix). NCI-H358 cells were obtained from ATCC (CRL-5807) and maintained in high-glucose (4.5 g/L) DMEM (Gibco 11995073) supplemented with 4 mM L-glutamine, 1 mM sodium pyruvate, and 10% heat-inactivated fetal bovine serum (Axenia Biologix). A375 cells were obtained from ATCC (CRL-1619) and maintained in high-glucose (4.5 g/L) DMEM (Gibco 11995073) supplemented with 4 mM L-glutamine, 1 mM sodium pyruvate, and 10% heat-inactivated fetal bovine serum (Axenia Biologix). SW1990 cells were obtained from ATCC (CRL-2172) and maintained in high-glucose (4.5 g/L) DMEM (Gibco 11995073) supplemented with 4 mM L-glutamine, 1 mM sodium pyruvate, and 10% heat-inactivated fetal bovine serum (Axenia Biologix).

Cells were passed for at least two generations after cryorecovery before they were used for assays. All cell lines were tested mycoplasma negative using MycoAlert™ Mycoplasma Detection Kit (Lonza). When indicated, cells were treated with drugs at 40-60% confluency at a final DMSO concentration of 1%. At the end of treatment period, cells were placed on ice. Unless otherwise indicated, adherent cells were washed once with ice-cold PBS (1 mL), scraped with a spatula, and pelleted by centrifugation (500×g, 5 min). Suspension cells were pelleted by centrifugation (500×g, 5 min), washed with 1 mL ice-cold PBS, and pelleted again. Cells were lysed in RIPA buffer supplemented with protease and phosphatase inhibitors (cOmplete and phosSTOP, Roche) on ice for 10 min. For RBD pulldown experiments, cells were lysed in Co-IP Lysis Buffer in lieu of RIPA buffer. Lysates were clarified by high-speed centrifugation (19,000×g, 10 min). Concentrations of lysates were determined with protein BCA assay (Thermo Fisher) and adjusted to 2 mg/mL with additional RIPA buffer (or Co-IP Lysis Buffer). Samples were mixed with 5×SDS Loading Dye and heated at 95° C. for 5 min.

### Analysis of GTP-Bound Ras by Raf-RBD Pulldown

Ras·GTP pulldown was performed with GST-RBD agarose beads (Cytoskeleton, 10 μL, 30 μg loaded GST-RBD). 50 μL lysate (2 mg/mL) was mixed with 10 μL beads, and the suspension was incubated at 4° C. for 1 h with constant end-to-end rotation. The beads were settled by centrifugation (500×g, 5 min) and the supernatant was carefully removed. The beads were washed with 2×500 μL ice-cold Co-IP wash buffer. Bound protein was eluted with 10 μL 2×SDS Loading Buffer.

### Gel Electrophoresis and Immunoblot

Unless otherwise noted, SDS-PAGE was run with Novex 16% Bis-Tris gel (Invitrogen) in MES running buffer (Invitrogen) at 200 V for 60 min following the manufacturer's instructions. Protein bands were transferred onto 0.2-μm nitrocellulose membranes (Bio-Rad) using a wet-tank transfer apparatus (Bio-Rad Criterion Blotter) in lx TOWBIN buffer with 10% methanol at 75V for 45 min. Membranes were blocked in 5% BSA-TBST for 1 h at 23° C. Primary antibody binding was performed with the indicated antibodies diluted in 5% BSA-TBST at 4° C. for at least 16 h. After washing the membrane three times with TBST (5 min each wash), secondary antibodies (goat anti-rabbit IgG-IRDye 800 and goat anti-mouse IgG-IRDye 680, Li-COR) were added as solutions in 5% skim milk-TBST at the dilutions recommended by the manufacturer. Secondary antibody binding was allowed to proceed for 1 h at 23° C. The membrane was washed three times with TBST (5 min each wash) and imaged on a Li-COR Odyssey fluorescence imager.

### Preparation of Mouse Stem Cell Virus (MSCV)

pMSCV-Puro plasmids containing full length human KRAS genes (wildtype, G12S, G12C) were constructed using standard molecule biology techniques by inserting the KRAS gene fragment between the BamHI and XhoI sites. Transfection-grade plasmids were prepared using ZymoPure II Plasmid Midiprep kit. EcoPack 293 cells (Takara Bio) were plated in 6-well plates (3×10⁵/mL, 2 mL). The next day, cells were transfected with 2.5 μg pMSCV plasmid using lipofectamine 3000 following the manufacturer's instructions. The cells were incubated for 66 h, and then the virus-containing supernatants were collected and passed through a 0.22-μm syringe filter. The harvested virus was used immediately for spinfection of Ba/F3 cells or stored at −80° C.

### Generation of Stable Ba/F3 Transductants

1 mL of MSCV-containing supernatant (vide supra) was added to one well of a 6-well plate containing 1×10⁶ Ba/F3 cells in 1 mL of media comprised of 60% RMPI 1640, 40% heat-inactivated FBS, 10 ng mouse IL-3 and 4 μg polybrene. Cells were spinfected by centrifugation at 2,000 g for 90 minutes at room temperature and then placed in the incubator for 24 hours. After 1 day, the cells were diluted into 10 mL culture medium (RPMI 1640+10% heat-inactivated FBS, 10 ng/mL mouse IL-3) and recovered for a second day after spinfection. On the third day after spinfection, cells were pelleted at 500×g for 5 min and resuspended in 10 mL selection medium (RPMI 1640+10% heat-inactivated FBS, 10 ng/mL mouse IL-3, 1.25 μg/mL puromycin). Cells were maintained under puromycin selection for 4-7 days, splitting as required to maintain density <2×10⁶ cells/mL. After 7 days, cells were pelleted, washed once with IL-3 free culture medium (RPMI 1640+10% heat-inactivated FBS) and pelleted again before resuspending at 2-4×10⁵ cells/mL in IL-3 free culture medium. Cells were maintained under these conditions for 7 days, passaging as needed to maintain density <2×10⁶ cells/mL. Growth was monitored (Countess II Cell Counter) over these 7 days to confirm that an IL-3 independent population has been achieved.

### Phospho-ERK Assay

Ba/F3 parental cells were pelleted and resuspend in IL-3 supplemented growth medium (RPMI 1640, 10% heat-inactivated FBS, 10 ng/mL mouse IL-3 medium) to 1×10⁷/mL. Ba/F3 K-Ras(G12S) transductant cells were pelleted and resuspended in IL-3 free growth medium (RPMI 1640, 10% heat-inactivated FBS) to 1×10⁷/mL. 25 μL cells were added to each well of a 96-well plate (2.5×10⁵ cells/well), followed by 5 μL 6× compound solutions (6% DMSO). The mixture was incubated at 37° C. for 1 h. Cell lysis and subsequent high-throughput phospho-ERK assay was performed using Cisbio Advanced phospho-ERK (Thr202/Tyr204) cellular kit (PerkinElmer 64AERPEG) following the manufacturer's instructions.

### GTPase Assay

GTPase assay was performed using EnzCheck Phosphate Assay Kit (Invitrogen E6646) using a previously reported procedure (1) with modifications. K-Ras proteins were loaded with GTP as follows. K-Ras proteins were diluted in EDTA Buffer (25 mM HEPES 7.5, 150 mM NaCl, 5 mM EDTA, 1 mM DTT) supplemented with 1 mM GTP at 0° C. to 100 μM. After incubation for 1 h on ice, the protein solutions were exchanged into Reaction Buffer (20 mM HEPES 7.5, 150 mM NaCl, 1 mM DTT) using a PD-10 column following the manufacturer's instructions. The eluted protein (3.5 mL) was concentrated using a 10K-MWCO Amicon-4 concentrator and protein concentrations were adjusted to 100 μM with Reaction Buffer. GTPase assay was performed in a clear 96-well half-volume UV-star plate (Greiner Bio-one 675801) as follows. To each well was added the following components: 50 μL of protein at 100 μM, 20 μL of 2-amino-6-mercapto-7-methylpurine riboside (MESG) at 1.0 mM, 5 μL of purine nucleotide phosphorylase at 0.1 U/μL. Control conditions where the protein solution was substituted for Reaction Buffer (“blank control”) or 100 μM free phosphate standard (“Pi control”) were also included. GTPase reaction was initiated by the addition of 25 μL 4×Mg Buffer (40 mM MgCl₂) or 4×NF1 buffer (200 μM NF1-GRD, 40 mM MgCl₂). This should be completed within 15 seconds. The absorbance at 360 nm was immediately read every 30 seconds for 3,600 seconds at 23° C. using a TECAN Spark 20M plate reader. For each data point, absorbance was subtracted with the reading in the blank control, then normalized to the difference between Pi control and blank control and reported as percentage of theoretical maximum hydrolysis.

### Sos- or EDTA-Mediated Nucleotide Exchange Assay

This assay was performed as previously reported (2-5) with slight modifications. BODIPY-GDP-loaded K-Ras proteins were prepared freshly as follows. To a 10 μM solution of K-Ras(wildtype)·GDP, K-Ras(G12S)·GDP, or K-Ras(G12S)·GDP·1′ in SEC Buffer (1 mL) was added sequentially BODIPY-GDP (5 mM, 40 μL, Thermo Fisher, final concentration 200 μM) and Na-EDTA pH 8.0 (0.5 M, 5 μL, final concentration 2.5 mM). The mixture was incubated at 23° C. for 1 h, and a solution of MgCl₂ (1.0 M, 20 μL, final concentration 10 mM) was added to the reaction mixture. The protein solution was run through a PD-10 column to remove the excess nucleotide following the manufacturer's protocol. Briefly, sample (˜1.0 mL) and excess buffer (1.5 mL) were loaded onto the column (equilibrated with NucEx Buffer), and desalted protein was eluted with NucEx Buffer (3.5 mL). Protein concentration was measured with Bradford assay and adjusted to 1.25 μM with NucEx Buffer. 12 μL of this solution (triplicate for each condition) was added to wells of a black 384-well low-volume assay plate (Corning 4514). 3 μL of either 1 mM GDP, 1 mM GDP+5 μM Sos, or 1 mM GDP+40 mM EDTA (all prepared in NucEx Buffer) was added via a multichannel pipet rapidly to the wells. This should take less than 15 s to finish. The plate was immediately placed in a TECAN Spark 20M plate reader, and fluorescence for BODIPY (excitation 488 nm, emission 520 nm) was read every 30 s over 1 h. Fluorescence intensity was normalized to values at time 0 and plotted again time. Observed rate constant (k_{obs}) was derived by fitting the curve to first-order kinetic equation

$$F = ( {F}_{0} - {F}_{∞} ) exp [ - {k}_{o b s} t ] + {F}_{∞}$$

and plotted against time.

### Differential Scanning Fluorimetry

The protein of interest was diluted with SEC Buffer [20 mM HEPES 7.5, 150 mM NaCl, 1 mM MgCl₂] to 8 μM. This solution was dispensed into wells of a white 96-well PCR plate in triplicate (25 μL/well). Fluorescence was measured at 0.5-° C. temperature intervals every 30 s from 25° C. to 95° C. on a Bio-Rad CFX96 qPCR system using the FRET setting. Each data set was normalized to the highest fluorescence and the normalized fluorescence reading was plotted against temperature in GraphPad Prism 8.0. Tm values were determined as the temperature(s) corresponding to the maximum(ma) of the first derivative of the curve.

### Detection of Covalent Modification of K-Ras Proteins by Mass Spectrometry

Test compounds were prepared as 100× stock solutions in DMSO. K-Ras proteins were diluted with SEC Buffer (20 mM HEPES 7.5, 150 mM NaCl, 1 mM MgCl₂) to 400 nM or 1 μM. In a typical reaction, 0.5 μL 100× compound stock was mixed with 50 μL diluted K-Ras protein, and the resulting mixture was incubated for the desired amount of time. The extent of modification was assessed by electrospray mass spectrometry using a Waters Xevo G2-XS system equipped with an Acquity UPLC BEH C4 1.7 μm column. The mobile phase was a linear gradient of 5-95% acetonitrile/water+0.05% formic acid. For kinetic measurements, a 2× compound solution was first prepared in SEC Buffer, which was then mixed with 400 nM K-Ras(G12S) protein at 1:1 (v/v) ratio. Injection time stamps were used to calculate elapsed time.

### Cell Viability Assay

Cells were seeded into 96-well white flat bottom plates (1,000 cells/well) (Corning) and incubated overnight. Cells were treated with the indicated compounds in a nine-point threefold dilution series (100 μL final volume) and incubated for 72 h. Cell viability was assessed using a commercial CellTiter-Glo (CTG) luminescence-based assay (Promega). Briefly, the 96-well plates were equilibrated to room temperature before the addition of diluted CTG reagent (100 L) (1:4 CTG reagent:PBS). Plates were placed on an orbital shaker for 30 min before recording luminescence using a Spark 20M (Tecan) plate reader.

### Recombinant Protein Expression and Purification

K-Ras (wildtype), K-Ras (G12S) and K-Ras (G12S) Cyslight

DNA sequences encoding human K-Ras (wildtype, aa 1-169), human K-Ras (G12S, aa 1-169), human K-Ras G12S Cyslight (G12S/C51S/C80L/C118S, aa 1-169), human NF1-GRD (aa 1203-1530) were codon optimized, synthesized by Twist Biosciences and cloned into pJExpress411 vector using the Gibson Assembly method (6). The resulting construct contains N-terminal 6×His tag and a TEV cleavage site (ENLYFQG). The proteins were expressed and purified following previously reported protocols (2, 7). Briefly, chemically competent BL21(DE3) cells were transformed with the corresponding plasmid and grown on LB agar plates containing 50 μg/mL kanamycin. A single colony was used to inoculate a culture at 37° C., 220 rpm in terrific broth containing 50 μg/mL kanamycin. When the optical density reached 0.6, the culture temperature was reduced to 20° C., and protein expression was induced by the addition of IPTG to 1 mM. After 16 h at 20° C., the cells were pelleted by centrifugation (6,500×g, 10 min) and lysed in lysis buffer [20 mM Tris 8.0, 500 mM NaCl, 5 mM imidazole] with a high-pressure homogenizer (Microfluidics, Westwood, MA). The lysate was clarified by high-speed centrifugation (19,000×g, 15 min) and the supernatant was used in subsequent purification by immobilized metal affinity chromatography (IMAC). His-TEV tagged protein was captured with Co-TALON resin (Clonetech, Takara Bio USA, 2 mL slurry/liter culture) at 4° C. for 1 h with constant end-to-end mixing. The loaded beads were then washed with lysis buffer (50 mL/liter culture) and the protein was eluted with elution buffer [20 mM Tris 8.0, 300 mM NaCl, 300 mM imidazole]. To this protein solution was added His-tagged TEV protease (0.05 mg TEV/mg Ras protein) and GDP (1 mg/mg Ras protein), and the mixture was dialyzed against TEV Cleavage Buffer [20 mM Tris 8.0, 300 mM NaCl, 1 mM EDTA, 1 mM DTT] at 4° C. using a 10K MWCO dialysis cassette until LC-MS analysis showed full cleavage (typically 16-24 h). MgCl₂ was added to a final concentration of 5 mM, and the mixture was incubated with 1 mL Ni-NTA (Qiagen) beads at 4° C. for 1 h to remove TEV protease, any residual His-tagged proteins and peptides. The protein solution was diluted 1:10 v/v with 20 mM Tris 8.0 and further purified with anion exchange chromatography (HiTrapQ column, GE Healthcare Life Sciences) using a NaCl gradient of 50 mM to 500 mM in 20 mM Tris 8.0. Nucleotide loading was performed by mixing the ion exchange-purified protein with an excess of GDP (5 mg/liter culture) or GppNHp (5 mg/liter culture) and 5 mM EDTA at 23° C. for 30 min. The reaction was stopped by the addition of MgCl₂ to 10 mM. For GppNHp, an additional calf intestine phosphatase treatment was performed as follows to ensure high homogeneity of the loaded nucleotide. The protein buffer was exchanged into Phosphatase Buffer [32 mM Tris 8.0, 200 mM ammonium sulfate, 0.1 mM ZnCl₂] with a HiTrap Desalting Column (GE Healthcare Life Sciences). To the buffer-exchanged protein solutions, GppNHp was added to 5 mg/mL, and Calf Intestine Phosphatase (NEB) was added to 10 U/mL. The reaction mixture was incubated on ice for 1 h, and MgCl₂ was added to a final concentration of 20 mM. After nucleotide loading, the protein was concentrated using an 10K MWCO centrifugal concentrator (Amicon-15, Millipore) to 20 mg/mL and purified by size exclusion chromatography on a Superdex 75 10/300 GL column (GE Healthcare Life Sciences). Fractions containing pure biotinylated Ras protein were pooled and concentrated to 20 mg/mL and stored at −78° C. In our hands, this protocol gives a typical yield of 5-15 mg/liter culture.

### NF1-GRD

DNA sequence encoding human NF1-GRD (aa 1203-1530) was codon optimized, synthesized by Twist Biosciences and cloned into pJExpress411 vector using the Gibson Assembly method. The resulting construct contains N-terminal 6×His tag and a TEV cleavage site (ENLYFQG (SEQ ID NO:50)). Protein was expressed and purified using the identical protocol as for the Ras proteins, except that the ion exchange and nucleotide loading steps were omitted. In our hands, this protocol gives a typical yield of 5-15 mg/liter culture.

### Sos^{cat}

The catalytic domain of Sos (residues 466-1049, Sos^{cat}) was expressed and purified following a published protocol (8).

### Crystallization

K-Ras(G12S) Cyslight (G12S/C51S/C80L/C118S) bound by GDP purified by size exclusion chromatography was diluted to 100 μM in Reaction Buffer (20 mM HEPES 7.5, 150 mM NaCl, 1 mM MgCl₂). Compound 1′ was added as a 10 mM solution in DMSO to a final concentration of 200 μM. The mixture was allowed to stand at 23° C. until LC-MS analysis of the reaction mixture showed full conversion to a single covalent adduct. The reaction mixture was purified by size exclusion chromatography (Superdex75, 20 mM HEPES 7.5, 150 mM NaCl, 1 mM MgCl₂) and concentrated to 20 mg/mL. For crystallization, 0.1 μL of the protein was mixed with 0.1 μL well buffer containing 0.1 M sodium acetate 4.6, 30% w/v PEG MME 2K, 0.2 M ammonium sulfate. Crystals were grown at 20° C. in a 96-well plate using the hanging-drop vapor diffusion method. Maximal crystal growth was achieved after 7 days. The crystals were transferred to a cryoprotectant solution (0.1 M sodium acetate 4.6, 30% w/v PEG MME 2K, 0.2 M ammonium sulfate, 20% glycerol) and flash-frozen in liquid nitrogen.

### X-Ray Data Collection and Structure Determination

Dataset was collected at the Advanced Light Source beamline 8.2.2 with X-ray at a wavelength of 0.999907 Å. The dataset was indexed and integrated using iMosfim (Battye et al., 2011), scaled with Scala (Evans, 2006) and solved by molecular replacement using Phaser (McCoy et al., 2007) in CCP4 software suite (Winn et al., 2011). The crystal structure of GDP-bound K-Ras(G12C)-MRTX849 adduct (PDB code: 6USZ) was used as the initial model. The structure was manually refined with Coot (Emsley et al., 2010) and PHENIX (Adams et al., 2010).

TABLE 1
List of antibodies.

| Target | Supplier | Identifier | Dilution |
|---|---|---|---|
| P-AKT [S473] | Cell Signaling Technology | 4060 | 1:1000 |
| P-AKT [T308] | Cell Signaling Technology | 4056 | 1:1000 |
| AKT | Cell Signaling Technology | 2920 | 1:1000 |
| P-ERK [T202/Y204] | Cell Signaling Technology | 9101 | 1:1000 |
| Total ERK | Cell Signaling Technology | 4695 | 1:1000 |
| Pan-Ras | Abcam | 108602 | 1:5000 |
| Ras(G12S) | NewEast Bio | 26186 | 1:1000 |
| GAPDH | Proteintech | 60004-1-Ig | 1:50000 |

TABLE 2
List of buffer compositions.

| Name | Composition |
|---|---|
| RIPA Buffer | 25 mM Tris 7.4 |
| | 150 mM NaCl |
| | 0.1% SDS |
| | 1% NP-40 |
| | 0.5% sodium deoxycholate |
| Co-IP Lysis Buffer | 50 mM HEPES 7.4 |
| (for NBD Pulldown) | 120 mM NaCl |
| | 1% NP-40 |
| | 1 mM MgCl₂ |
| Co-IP Wash Buffer | 50 mM HEPES 7.4 |
| (for NBD Pulldown) | 120 mM NaCl |
| | 0.01% NP-40 |
| | 1 mM MgCl2 |
| 5× SDS | 250 mM Tris 6.8 |
| Loading Buffer | 500 mM DTT |
| | 10% w/v SDS |
| | 0.1% w/v Bromophenol Blue |
| | 50% Glycerol |
| TOWBIN Buffer | 25 mM Tris |
| | 192 mM Glycine |
| | pH 8.3 |
| Lysis Buffer | 20 mM Tris 8.0 |
| | 500 mM NaCl |
| | 5 mM imidazole |
| Elution Buffer | 20 mM Tris 8.0 |
| | 300 mM NaCl |
| | 300 mM imidazole |
| TEV Cleavage | 20 mM Tris 8.0 |
| Buffer | 300 mM NaCl |
| | 1 mM EDTA |
| | 1 mM DTT |
| Phosphatase Buffer | 32 mM Tris 8.0 |
| | 200 mM ammonium sulfate |
| | 0.1 mM ZnCl₂ |
| SEC Buffer | 20 mM HEPES 7.5 |
| | 150 mM NaCl |
| | 1 mM MgCl₂ |
| Nucleotide | 20 mM HEPES 7.5 |
| Exchange Buffer | 150 mM NaCl |
| | 1 mM MgCl₂ |
| | 1 mM DTT |
| GTPase Reaction | 20 mM HEPS 7.5 |
| Buffer | 150 mM NaCl |
| | 1 mM DTT |

List of Recombinant Protein Sequences Used in this Study

K-Ras wildtype
(SEQ ID NO: 51)
MHHHHHHSSGRENLYFQGMTEYKLVVVGAGGVGKSALTIQLIQNHFVDE
YDPTIEDSYRKQVVIDGETCLLDILDTAGQEEYSAMRDQYMRTGEGFLC
VFAINNTKSFEDIHHYREQIKRVKDSEDVPMVLVGNKCDLPSRTVDTKQ
AQDLARSYGIPFIETSAKTRQGVDDAFYTLVREIRKHKEK
K-Ras wildtype CysLight
(SEQ ID NO: 52)
MHHHHHHSSGRENLYFQGMTEYKLVVVGAGGVGKSALTIQLIQNHFVDE
YDPTIEDSYRKQVVIDGETSLLDILDTAGQEEYSAMRDQYMRTGEGFLL
VFAINNTKSFEDIHHYREQIKRVKDSEDVPMVLVGNKSDLPSRTVDTKQ
AQDLARSYGIPFIETSAKTRQGVDDAFYTLVREIRKHKEK
K-Ras G12S
(SEQ ID NO: 53)
MHHHHHHSSGRENLYFQGMTEYKLVVVGASGVGKSALTIQLIQNHFVDE
YDPTIEDSYRKQVVIDGETCLLDILDTAGQEEYSAMRDQYMRTGEGFLC
VFAINNTKSFEDIHHYREQIKRVKDSEDVPMVLVGNKCDLPSRTVDTKQ
AQDLARSYGIPFIETSAKTRQGVDDAFYTLVREIRKHKEK
K-Ras G12S CysLight
(SEQ ID NO: 54)
MHHHHHHSSGRENLYFQGMTEYKLVVVGASGVGKSALTIQLIQNHFVDE
YDPTIEDSYRKQVVIDGETSLLDILDTAGQEEYSAMRDQYMRTGEGFLL
VFAINNTKSFEDIHHYREQIKRVKDSEDVPMVLVGNKSDLPSRTVDTKQ
AQDLARSYGIPFIETSAKTRQGVDDAFYTLVREIRKHKEK
NF1-GRD
(SEQ ID NO: 55)
MHHHHHHSSGRENLYFQGDRFERLVELVTMMGDQGELPIAMALANVVPC
SQWDELARVLVTLFDSRHLLYQLLWNMFSKEVELADSMQTLFRGNSLAS
KIMTFCFKVYGATYLQKLLDPLLRIVITSSDWQHVSFEVDPTRLEPSES
LEENQRNLLQMTEKFFHAIISSSSEFPPQLRSVCHCLYQVVSQRFPQNS
IGAVGSAMFLRFINPAIVSPYEAGILDKKPPPRIERGLKLMSKILQSIA
NHVLFTKEEHMRPFNDFVKSNFDAARRFFLDIASDCPTSDAVNHSLSFI
SDGNVLALHRLLWNNQEKIGQYLSSNRDHKAVGRRPFDKMATLLAYLGP
PEH

### Chemical Synthesis

### General Experiment Procedure

All reactions were performed in oven-dried glassware fitted with rubber septa under a positive pressure of argon, unless otherwise noted. Air- and moisture-sensitive liquids were transferred via syringe. Solutions were concentrated by rotary evaporation at or below 40° C. Analytical thin-layer chromatography (TLC) was performed using glass plates pre-coated with silica gel (0.25-mm, 60-A pore size, 230-400 mesh, Merck KGA) impregnated with a fluorescent indicator (254 nm). TLC plates were visualized by exposure to ultraviolet light (UV), then were stained by submersion in a 10% solution of phosphomolybdic acid (PMA) in ethanol or an acidic ethanolic solution of p-anisaldehyde (this solution was prepared by sequential additions of concentrated sulfuric acid (5.0 mL), glacial acetic acid (1.5 mL) and p-anisaldehyde (3.7 mL) to absolute ethanol (135 mL) at 23° C. with efficient stirring), followed by brief heating on a hot plate. Flash column chromatography was performed with Teledyne ISCO CombiFlash EZ Prep chromatography system, employing pre-packed silica gel cartridges (Teledyne ISCO RediSep).

### Solvents and Reagents

Anhydrous solvents were purchased from Acros Organics. Unless specified below, all chemical reagents were purchased from Sigma-Aldrich, AK Scientific or Chemscene. Commercial solvents and reagents were used as received.

### Instrumentation

Proton nuclear magnetic resonance (¹H NMR) spectra, carbon nuclear magnetic resonance (¹³C NMR) spectra, and fluorine nuclear magnetic resonance (¹⁹F NMR) spectra were recorded on Bruker AvanceIII HD instrument (400 MHz/100 MHz/376 MHz) at 23° C. Proton chemical shifts are expressed in parts per million (ppm, δ scale) and are referenced to residual protium in the NMR solvent (CHCl₃: δ 7.26, D₂HCOD: δ 3.31). Carbon chemical shifts are expressed in parts per million (ppm, δ scale) and are referenced to the carbon resonance of the NMR solvent (CDCl₃: δ 77.0, CD₃OD: δ 49.0). Fluorine chemical shifts are expressed in parts per million (ppm, δ scale) and are referenced to an external standard of trifluoroacetic acid (−76.55 ppm). Data are represented as follows: chemical shift, multiplicity (s=singlet, d=doublet, t=triplet, q=quartet, dd=doublet of doublets, dt=doublet of triplets, m=multiplet, br=broad, app=apparent), integration, and coupling constant (J) in Hertz (Hz). High-resolution mass spectra were obtained using a Waters Xevo G2-XS time-of-flight mass spectrometer.

### Mini-Workup

When a mini-workup (A/B) is indicated in the procedure, it was performed as follows: an aliquot (5 μL) of the reaction mixture was retrieved with a glass pipet and added to a plastic vial containing 0.2 mL organic solvent A and 0.2 mL aqueous solution B. The vial was shaken vigorously and allowed to stand until the two layers partitioned. The organic layer was then used for TLC or LC-MS analysis as specified in the procedure.

### Monitoring Reaction Progress by LC-MS

When LC-MS analysis of the reaction mixture is indicated in the procedure, it was performed as follows. An aliquot (1 μL) of the reaction mixture (or the organic phase of a mini-workup mixture) was diluted with 100 μL 1:1 acetonitrile:water. 1 μL of the diluted solution was injected onto a Waters Acquity UPLC BEH C18 1.7 μm column and eluted with a linear gradient of 5-95% acetonitrile/water (+0.1% formic acid) over 3.0 min. Chromatograms were recorded with a UV detector set at 254 nm and a time-of-flight mass spectrometer (Waters Xevo G2-XS).

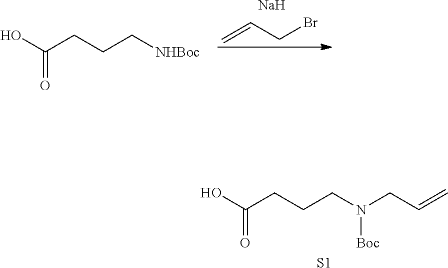

### 4-(allyl(tert-butoxycarbonyl)amino)butanoic acid (S1)

A solution of 4-[(tert-butoxycarbonyl)amino]butanoic acid (5.00 g, 24.6 mmol, 1 equiv.) in THF (10.0 mL) was added dropwise to a suspension of sodium hydride (60% in mineral oil, 2.46 g, 2.50 equiv.) in THF (15.0 mL) at 23° C. After gas evolution had subsided, allyl bromide (3.19 mL, 36.9 mmol, 1.50 equiv.) was added dropwise via syringe. The resulting mixture was stirred at 23° C. for 24 h. 10% Citric acid was added dropwise until the pH of the solution reached 5. Care must be taken at the beginning of the addition as the excess sodium hydride may react violently with water if the solution is added too fast. The reaction mixture was extracted with ether (3×20 mL). The combined ether layers were washed with water (2×30 mL) and saturated aqueous sodium chloride solution (30 mL). The washed solution was dried over magnesium sulfate, and the dried solution was concentrated. The residue was purified by column chromatography (20-50% ethyl acetate-hexanes) to afford the product as a colorless oil (4.70 g, 78.5%).

¹H NMR (400 MHz, CDCl₃) δ 5.79 (ddt, J=16.3, 11.0, 5.7 Hz, 1H), 5.19-5.10 (m, 2H), 3.82 (br s, 2H), 3.30 (br s, 2H), 2.39 (t, J=7.1 Hz, 2H), 1.87 (p, J=7.0 Hz, 2H), 1.48 (s, 9H). ¹³C NMR (100 MHz, CDCl₃) δ 178.53, 155.70 (br), 133.93, 116.55 (br), 79.93, 49.52 (br), 45.63, 31.22 (br), 28.34, 23.31. HRMS (C₁₂H₂₁NO₄+Na)⁺ Calc'd: 266.1368, Found: 266.1383.

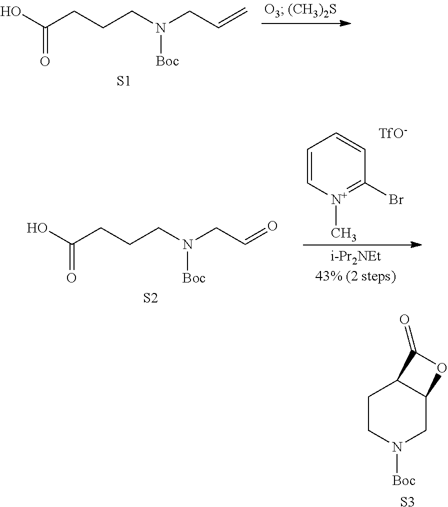

### 4-((tert-butoxycarbonyl)(2-oxoethyl)amino)butanoic acid (S2)

A solution of S1 (4.70 g, 19.3 mmol, 1 equiv.) in dichloromethane (96.5 mL) was cooled to −78° C. (dry ice/acetone bath). A stream of ozone was bubbled through the solution until a persistent pale blue color was observed. Dimethyl sulfide (7.09 mL, 96.5 mmol, 5.00 equiv.) was added to the reaction mixture, and the solution was allowed to warm to 23° C. The resulting mixture was stirred for 24 h at 23° C. The reaction mixture was directly washed with water (100 mL), and the washed organic layer was dried over sodium sulfate. The dried solution was filtered and concentrated under reduced pressure to afford the product as a colorless oil. The product was used without further purification.

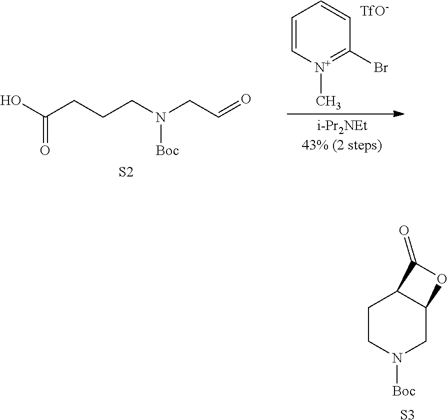

### tert-butyl (1R,6R)-7-oxo-8-oxa-3-azabicyclo[4.2.0]octane-3-carboxylate (S3)

A solution of S2 (800 mg, 3.26 mmol, 1 equiv.) in acetonitrile (32.62 mL) was added dropwise over 1 h to a stirred solution of 2-bromo-1-methylpyridinium trifluoromethanesulfonate (3.15 g, 9.78 mmol, 3.00 equiv.), quinine acetate (239 mg, 0.652 mmol, 0.200 equiv.), and triethylamine (1.82 mL, 13.05 mmol, 4.00 equiv.) in acetonitrile (32.6 mL) at 23° C. The resulting mixture was further stirred at 23° C. for 22 h. The reaction mixture was directly concentrated, and the residue was purified by column chromatography (30-100% ethyl acetate-hexanes) to afford the product as a colorless oil, which solidified upon standing (322 mg, 43.4%).

¹H NMR (1:1 mixture of rotamers, some peaks are resolved for the two rotamers, 400 MHz, CDCl₃) δ 4.82 (br s, 0.5H), 4.77 (br s, 0.5H), 4.41 (br d, J=15.6 Hz, 0.5H), 4.28 (br d, J=15.6 Hz, 0.5H), 3.90-3.82 (m, 1H), 3.61 (s, 1H), 3.52-3.28 (m, 2H), 2.25-2.06 (m, 1H), 2.06-1.86 (m, 1H), 1.48 (s, 9H). ¹³C NMR (1:1 mixture of rotamers, some peaks are resolved for the two rotamers, 100 MHz, CDCl₃) δ 169.66 (br), 155.27 (br), 80.41, 69.32 (br), 68.97 (br), 47.54 (br), 42.32 (br), 40.87 (br), 40.06 (br), 39.15(br), 28.38, 19.83. HRMS (C₁₁H₁₇NO₄+H−tBu)⁺ Calc'd: 172.0610, Found: 172.0621.

The racemate of this compound was prepared in a similar manner except that quinine acetate was omitted from the reaction mixture.

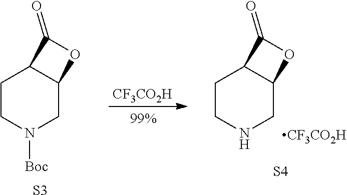

### (1R,6R)-8-oxa-3-azabicyclo[4.2.0]octan-7-one trifluoroacetate (S4)

S3 (200 mg, 0.880 mmol, 1 equiv.) was dissolved in 1:1 trifluoroacetic acid:dichloromethane (1.0 mL). The resulting yellow solution was allowed to stand at 0° C. for 1 h, then was concentrated in vacuo. The resulting yellow oil was triturated with anhydrous ether (10 mL) and the resulting solids were collected by centrifugation. Drying under vacuum afforded the product as a yellow powder (210 mg, 99.0%).

¹H NMR (400 MHz, MeOD) δ 4.99 (ddd, J=5.9, 3.4, 2.0 Hz, 1H), 4.11 (td, J=6.8, 3.8 Hz, 1H), 3.73 (dd, J=15.1, 2.1 Hz, 1H), 3.58 (dd, J=15.1, 3.4 Hz, 1H), 3.38-3.20 (m, 2H), 2.35-2.13 (m, 2H). ¹³C NMR (100 MHz, MeOD) δ 169.33, 65.77, 45.81, 41.15, 38.29, 16.72. Signals from the trifluoroacetate anion were too weak to be observed. ¹⁹F NMR (376 MHz, MeOD) δ −76.92. HRMS (C₆H9NO₂+H)⁺ Calc'd: 128.0712, Found: 128.0707.

The racemate of this compound was prepared in a similar manner.

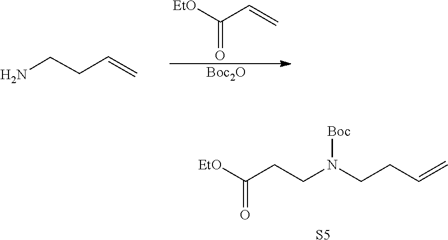

### Ethyl 3-(but-3-en-1-yl(tert-butoxycarbonyl)amino)propanoate (S5)

1-amino-3-butene hydrochloride (538 mg, 5 mmol, 1 equiv.) was added dropwise to a solution of ethyl acrylate (500 mg, 5.00 mmol, 1.00 equiv.) and triethylamine (697 μL, 5.00 mmol, 1.00 equiv) in THF (5.0 mL) at 23° C. The resulting mixture was stirred at 23° C. In 16 h, TLC analysis (100% ethyl acetate) showed full consumption of the amine starting material. The reaction mixture was concentrated in vacuo. THF (10 mL) was added to the residue, followed by triethylamine (697 μL, 5.00 mmol, 1.00 equiv.) and Di-tert-butyl dicarbonate (1.09 g, 5.00 mmol, 1.00 equiv.). Gas evolution immediately ensued and subsided over −20 min. The reaction mixture was concentrated, and the residue was purified by column chromatography (20-50% ethyl acetate-hexanes) to afford the intermediate Michael addition product as a colorless oil (650 mg, 47.9%).

¹H NMR (400 MHz, CDCl₃) δ 5.78 (td, J=17.0, 6.9 Hz, 1H), 5.17-5.00 (m, 2H), 4.16 (q, J=7.1 Hz, 2H), 3.48 (s, 2H), 3.28 (s, 2H), 2.58 (s, 2H), 2.35-2.25 (m, 2H), 1.48 (s, 9H), 1.28 (t, J=7.1 Hz, 3H).

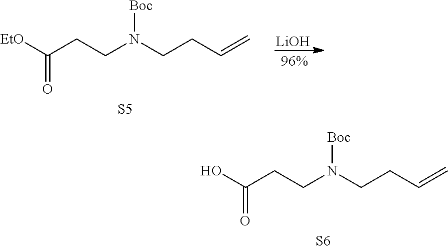

### 3-(but-3-en-1-yl(tert-butoxycarbonyl)amino)propanoic acid (S6)

Lithium hydroxide hydrate (64.8 mg, 1.54 mmol) was added to a solution of S5 (209 mg, 0.772 mmol, 1 equiv.) in 1:1 THF (1.5 mL):Water (1.5 mL) at 23° C. The resulting biphasic mixture was stirred at that temperature for 4 h. TLC analysis (50% ethyl acetate-hexanes) at this point showed full consumption of the ester starting material. 10% Citric acid (10 mL) was added to the reaction mixture. The reaction mixture was extracted with ether (3×10 mL). The combined organic layers were dried over magnesium sulfate. The dried solution was filtered, and the filtrate was concentrated to afford the product as a colorless oil (180 mg, 95.8%).

¹H NMR (400 MHz, CDCl₃) δ 5.78 (ddt, J=17.1, 10.2, 6.9 Hz, 1H), 5.13-5.02 (m, 2H), 3.50 (t, J=6.9 Hz, 2H), 3.31 (t, J=7.3 Hz, 2H), 2.67 (s, 2H), 2.29 (p, J=7.3 Hz, 2H), 1.49 (s, 9H). HRMS (C₁₂H₂₁NO₄+Na)⁺ Calc'd: 266.1368, Found: 266.1383.

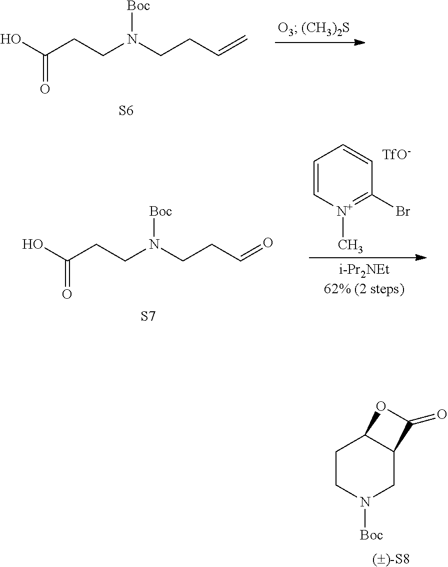

### 3-((tert-butoxycarbonyl)β-oxopropyl)amino)propanoic acid (S7)

Ozone was bubbled through a solution of S6 (246 mg, 1.01 mmol, 1 equiv.) in dichloromethane (4.52 mL) at −78° C. until a purple color persisted. The gas source was switched to argon, and bubbling was continued for 10 min to remove excess ozone. Dimethyl sulfide (0.74 mL, 10.1 mmol, 10.0 equiv.) was added, and the reaction mixture was allowed to warm to 23° C. and kept stirred at that temperature for 15 h. The reaction mixture was partitioned between water (5 mL) and dichloromethane (5 mL). The layers were separated, and the aqueous layer was extracted with dichloromethane (2×5 mL). The combined organic layers were dried over sodium sulfate. The dried solution was filtered, and the filtrate was concentrated to afford the product as a yellow oil. The product was used in the next reaction without further purification.

### Rac-tert-butyl (1S,6R)-8-oxo-7-oxa-3-azabicyclo[4.2.0]octane-3-carboxylate (±-S8)

A flame-dried 100-ml flask was charged with 2-bromo-1-methylpyridinium trifluoromethanesulfonate (969 mg, 3.01 mmol, 3.00 equiv), acetonitrile (17.1 mL), triethylamine (0.56 mL, 4.012 mmol, 4.00 equiv.) and a magnetic stir bar. A solution of S7 (246 mg, 1.00 mmol, 1 equiv.) in acetonitrile (6 mL) was added with a syringe pump at rate of 2 mL/min. After addition, the reaction solution was kept stirred at 23° C. for 16 h. The reaction mixture was concentrated in vacuo. The residue was partitioned between 0.5 M pH 7 phosphate buffer (10 mL) and dichloromethane (10 mL). The layers were separated, and the aqueous layer was extracted with dichloromethane (2×10 mL). The combined organic layers were dried over sodium sulfate, and the dried solution was concentrated. The residue was purified by column chromatography (10-100% ethyl acetate-hexanes) to afford the product as a colorless oil, which solidified upon standing (141 mg, 61.9%).

¹H NMR (1:1 mixture of rotamers, some peaks are resolved for the two rotamers, 400 MHz, CDCl₃) δ 4.89 (dt, J=6.0, 2.8 Hz, 1H), 4.24-4.08 (m, 1H), 3.90 (s, 0.5H), 3.84 (s, 0.5H), 3.63-3.44 (m, 2H), 3.63-3.44 (m, 2H), 3.38-3.25 (m, 1H), 2.42-2.25 (m, 1H), 2.18-2.01 (m, 1H), 1.45 (s, 9H). ¹³C NMR (100 MHz, CDCl₃) δ 169.80, 154.66 (br), 80.38 (br), 68.40, 68.27, 50.16, 49.84, 37.99, 36.95, 36.46, 35.68, 28.34, 25.31. HRMS (C₁₁H₁₇NO₄+Na)⁺ Calc'd: 250.1055, Found: 250.1060.

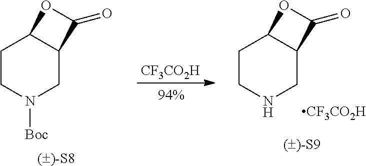

### Rac-(1S,6R)-7-oxa-3-azabicyclo[4.2.0]octan-8-one (±-S9)

Rac-tert-butyl (1S,6R)-8-oxo-7-oxa-3-azabicyclo[4.2.0]octane-3-carboxylate (±-S8) (200 mg, 0.880 mmol, 1 equiv.) was dissolved in 1:1 trifluoroacetic acid:dichloromethane (1.0 mL). The resulting yellow solution was allowed to stand at 0° C. for 1 h, then was concentrated in vacuo. The resulting yellow oil was triturated with anhydrous ether (10 mL) and the resulting solids were collected by centrifugation. Drying under vacuum afforded the product as a yellow powder (200 mg, 94.2%).

¹H NMR (400 MHz, MeOD) δ 5.02 (ddd, J=7.1, 4.3, 3.0 Hz, 1H), 4.20 (ddd, J=7.5, 6.7, 2.8 Hz, 1H), 3.59 (dd, J=14.2, 2.8 Hz, 1H), 3.47 (dd, J=14.2, 7.5 Hz, 1H), 3.35-3.23 (m, 2H), 2.48-2.32 (m, 2H). ¹³C NMR (100 MHz, MeOD) δ 168.92, 66.53, 44.69, 36.39, 35.77, 23.48. Signals from the trifluoroacetate anion were too weak to be observed. ¹⁹F NMR (376 MHz, MeOD) δ −76.92. HRMS (C₆H₉NO₂+H)⁺ Calc'd: 128.0712, Found: 128.0707.

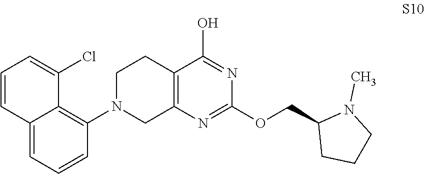 [SMILES: CN1CCC[C@H]1COc1nc(O)c2c(n1)CN(c1cccc3cccc(Cl)c13)CC2]

7-(8-chloro-1-naphthyl)-2-[[(2S)-1-methylpyrrolidin-2-yl]methoxy]-6,8-dihydro-5H-pyrido[3,4-d]pyrimidin-4-ol (S10) was prepared according to the protocol described in US Patent App. 2020/0331911.

¹H NMR (400 MHz, MeOD) δ 8.37 (s, 2H), 7.79 (d, J=8.1 Hz, 1H), 7.64 (d, J=8.1 Hz, 1H), 7.50 (d, J=7.4 Hz, 1H), 7.44 (t, J=7.8 Hz, 1H), 7.40-7.31 (m, 1H), 7.27 (dd, J=7.5, 2.3 Hz, 1H), 4.73 (td, J=13.1, 3.1 Hz, 1H), 4.61-4.50 (m, 1H), 4.04 (dd, J=17.5, 2.8 Hz, 1H), 3.86-3.67 (m, 2H), 3.62 (dd, J=17.4, 2.5 Hz, 1H), 3.48 (dd, J=12.6, 5.6 Hz, 1H), 3.19 (dt, J=11.2, 8.0 Hz, 1H), 3.09 (dd, J=10.6, 4.0 Hz, 1H), 3.04 (s, 3H), 2.89-2.76 (m, 1H), 2.54 (d, J=16.4 Hz, 1H), 2.40-2.26 (m, 1H), 2.22-2.03 (m, 2H), 2.03-1.89 (m, 1H). ¹³C NMR (100 MHz, MeOD) δ 166.56, 165.02, 158.63, 154.85, 154.82, 148.37, 148.33, 137.51, 129.53, 129.30, 128.17, 126.32, 125.71, 125.70, 125.34, 124.88, 124.85, 118.67, 118.63, 113.05, 113.03, 66.84, 65.02, 57.03, 56.91, 56.85, 49.61, 49.51, 40.01, 26.18, 26.16, 21.98, 21.95, 21.84, 21.78. HRMS (C₂₃H₂₅ClN₄O₂+H)⁺ Calc'd: 425.1744, Found: 425.1732.

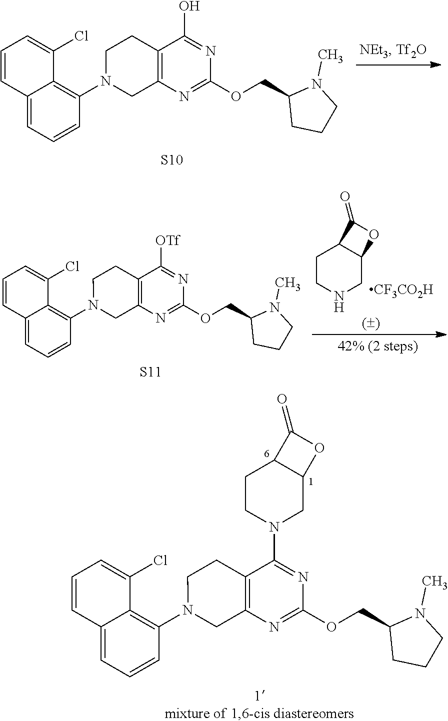

β-lactone 1′ (mixture of diastereomers), also referred to herein as 12-026.

Triethylamine (29.5 μL, 0.212 mmol, 3.00 equiv.) and triflic anhydride (14.2 μL, 0.0850 mmol, 1.20 equiv.) were added sequentially to a stirred solution of S10 (30.0 mg, 0.0710 mmol, 1 equiv.) in dichloromethane (0.18 mL) at 0° C. In 15 min, TLC analysis (10% methanol-dichloromethane) showed full conversion of the starting material to a slightly less polar spot. The reaction mixture was partitioned between saturated aqueous sodium bicarbonate solution (5 mL) and dichloromethane (5 mL). The layers were separated, and the aqueous layer was extracted with dichloromethane (2×5 mL). The combined organic layers were dried over sodium sulfate. The dried solution was filtered, and the filtrate was concentrated to afford the triflate as a crude product. DMF (0.350 mL) was added to the residue. Rac-(1R,6R)-7-oxa-4-azabicyclo[4.2.0]octan-8-one trifluoroacetic acid salt (34.0 mg, 0.141 mmol, 2.00 equiv.) was added to the resulting solution as a solid. Triethylamine (29.5 μL, 0.212 mmol, 3.00 equiv.) was added via syringe. The resulting brown mixture was stirred at 23° C. for 1 h. The residue was diluted with 50% acetonitrile-water to a volume of 2.0 mL, and the solution was filtered through a 0.45 μM PTFE syringe filter. The filtrate was loaded onto a Redi-Sep C18 30g Gold column equilibrated with 10% acetonitrile-water+0.1% formic acid, and the product was purified by column chromatography (10-100% acetonitrile-water+0.1% formic acid). The product-containing fractions were lyophilized to afford the product as a white solid (15.7 mg, 41.6%).

¹H NMR (400 MHz, CDCl₃) δ 7.80-7.72 (m, 1H), 7.67-7.56 (m, 1H), 7.56-7.40 (m, 2H), 7.39-7.32 (m, 1H), 7.27-7.16 (m, 1H), 5.04-4.81 (m, 1H), 4.49-4.30 (m, 3H), 4.22-4.12 (m, 1H), 4.06-3.68 (m, 3H), 3.66-3.43 (m, 2H), 3.39-2.90 (m, 3H), 2.87-2.58 (m, 2H), 2.54-2.46 (m, 3H), 2.41-2.23 (m, 2H), 2.16-2.00 (m, 1H), 1.90-1.73 (m, 3H). Note: Product was a mixture of diastereomers. NMR peaks were reported as seen. HRMS (C₂₉H₃₂ClN₅O₃+H)⁺ Calc'd: 534.2272, Found: 534.2305.

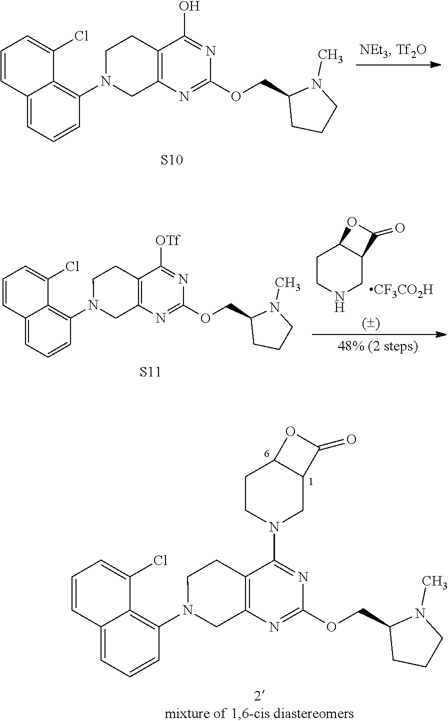

β-lactone 2′ (mixture of diastereomers), also referred to herein as 12-027.

Triethylamine (29.5 μL, 0.212 mmol, 3.00 equiv.) and triflic anhydride (14.2 μL, 0.0850 mmol, 1.20 equiv.) were added sequentially to a stirred solution of S10 (30.0 mg, 0.0710 mmol, 1 equiv.) in DCM (0.18 mL) at 0° C. In 15 min, TLC analysis (10% methanol-dichloromethane) showed full conversion of the starting material to a slightly less polar spot. The reaction mixture was partitioned between saturated aqueous sodium bicarbonate solution (5 mL) and dichloromethane (5 mL). The layers were separated, and the aqueous layer was extracted with dichloromethane (2×5 mL). The combined organic layers were dried over sodium sulfate. The dried solution was filtered, and the filtrate was concentrated to afford the triflate as a crude product. DMF (0.350 mL) was added to the residue. Rac-(1S,6R)-7-oxa-3-azabicyclo[4.2.0]octan-8-one trifluoroacetate salt (34.0 mg, 0.141 mmol, 2.00 equiv.) was added to the resulting solution as a solid. Triethylamine (29.5 μL, 0.212 mmol, 3.00 equiv.) was added via syringe. The resulting brown mixture was stirred at 23° C. for 1 h. The residue was diluted with 50% acetonitrile-water to a volume of 2.0 mL, and the solution was filtered through a 0.45 μM PTFE syringe filter. The filtrate was loaded onto a Redi-Sep C18 30g Gold column equilibrated with 10% acetonitrile-water+0.1 formic acid, and the product was purified by column chromatography (10-100% acetonitrile-water+0.1% formic acid). The product-containing fractions were lyophilized to afford the product as a white solid (18.0 mg, 47.7%).

¹H NMR (400 MHz, CDCl₃) δ 7.79-7.72 (m, 1H), 7.66-7.58 (m, 1H), 7.58-7.40 (m, 2H), 7.37-7.31 (m, 1H), 7.27-7.12 (m, 1H), 5.03-4.92 (m, 1H), 4.52-4.34 (m, 3H), 4.28-4.14 (m, 1H), 4.13-3.95 (m, 2H), 3.94-3.69 (m, 1H), 3.69-3.45 (m, 2H), 3.43-2.88 (m, 4H), 2.85-2.59 (m, 2H), 2.59-2.50 (m, 3H), 2.50-2.16 (m, 2H), 2.16-2.01 (m, 2H), 2.01-1.72 (m, 3H). Note: Product was a mixture of diastereoisomers. NMR peaks were reported as seen. HRMS (C₂₉H₃₂ClN₅O₃+H)⁺ Calc'd: 534.2272, Found: 534.2305.

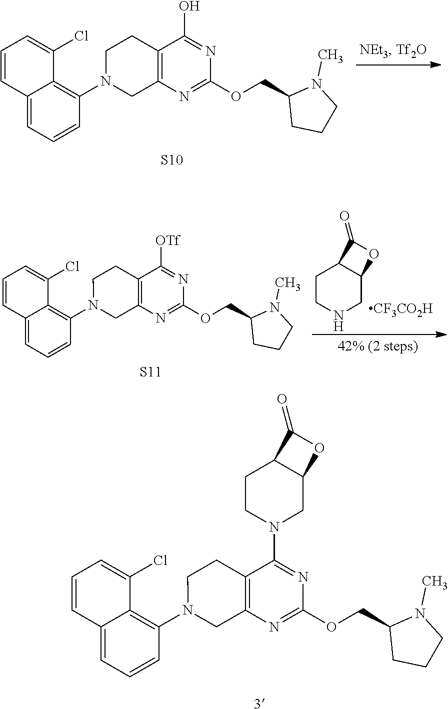

β-lactone 3′.

Triethylamine (29.5 μL, 0.212 mmol, 3.00 equiv.) and triflic anhydride (14.2 μL, 0.0850 mmol, 1.20 equiv.) were added sequentially to a stirred solution of S10 (30.0 mg, 0.0710 mmol, 1 equiv.) in DCM (0.18 mL) at 0° C. In 15 min, TLC analysis (10% methanol-dichloromethane) showed full conversion of the starting material to a slightly less polar spot. The reaction mixture was partitioned between saturated aqueous sodium bicarbonate solution (5 mL) and dichloromethane (5 mL). The layers were separated, and the aqueous layer was extracted with dichloromethane (2×5 mL). The combined organic layers were dried over sodium sulfate. The dried solution was filtered, and the filtrate was concentrated to afford the triflate as a crude product. DMF (0.350 mL) was added to the residue. (1R,6R)-7-oxa-4-azabicyclo[4.2.0]octan-8-one trifluoroacetic acid salt (34.0 mg, 0.141 mmol, 2.00 equiv.) was added to the resulting solution as a solid. Triethylamine (29.5 μL, 0.212 mmol, 3.00 equiv.) was added via syringe. The resulting brown mixture was stirred at 23° C. for 1 h. The residue was diluted with 50% acetonitrile-water to a volume of 2.0 mL, and the solution was filtered through a 0.45 μM PTFE syringe filter. The filtrate was loaded onto a Redi-Sep C18 30g Gold column equilibrated with 10% acetonitrile-water+0.1% formic acid, and the product was purified by column chromatography (10-100% acetonitrile-water+0.1% formic acid). The product-containing fractions were lyophilized to afford the product as a white solid (16.0 mg, 42.4%).

¹H NMR (400 MHz, CDCl₃) δ 7.80-7.70 (m, 1H), 7.67-7.58 (m, 1H), 7.57-7.41 (m, 2H), 7.35 (app t, J=7.8 Hz, 1H), 7.27-7.14 (m, 1H), 4.97-4.81 (m, 1H), 4.49-4.34 (m, 3H), 4.22-4.09 (m, 1H), 4.01-3.89 (m, 2H), 3.89-3.67 (m, 2H), 3.64-3.39 (m, 2H), 3.38-3.18 (m, 1H), 3.18-2.87 (m, 2H), 2.81-2.58 (m, 2H), 2.50 (app d, 3H), 2.41-2.22 (m, 2H), 2.16-2.01 (m, 2H), 1.92-1.70 (m, 3H). Note: Product appears as a mixture of conformational isomers. NMR peaks were reported as seen. HRMS (C₂₉H₃₂ClN₅O₃+H)⁺ Calc'd: 534.2272, Found: 534.2257.

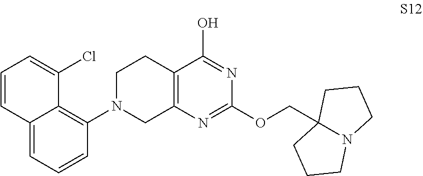 [SMILES: Oc1nc(OCC23CCCN2CCC3)nc2c1CCN(c1cccc3cccc(Cl)c13)C2]

7-(8-chloronaphthalen-1-yl)-2-((tetrahydro-1H-pyrrolizin-7a(5H)-yl)methoxy)-5,6,7,8-tetrahydropyrido[3,4-d]pyrimidin-4-ol (S12) was prepared according to the protocol described for compound S10, except where (2S)-1-methylpyrrolidine was substituted for 1,2,3,5,6,7-hexahydropyrrolizin-8-ylmethanol.

¹H NMR (formate salt, 400 MHz, MeOD) δ 8.30 (s, 2H), 7.83 (dd, J=8.2, 1.3 Hz, 1H), 7.68 (dd, J=8.2, 1.2 Hz, 1H), 7.56-7.44 (m, 2H), 7.42-7.31 (m, 2H), 4.63-4.43 (m, 2H), 4.07 (d, J=17.4 Hz, 1H), 3.75-3.62 (m, 3H), 3.55 (ddt, J=11.9, 5.9, 2.1 Hz, 1H), 3.28 (dt, J=12.1, 6.5 Hz, 2H), 3.17 (ddd, J=11.9, 10.3, 4.1 Hz, 1H), 2.93-2.79 (m, 1H), 2.60 (ddt, J=16.6, 4.1, 2.0 Hz, 1H), 2.32-2.02 (m, 8H). ¹³C NMR (formate salt, 100 MHz, MeOD) δ 165.60, 158.74, 155.07, 148.38, 137.55, 129.53, 129.28, 128.16, 126.31, 125.72, 125.33, 124.86, 118.63, 113.18, 79.65, 68.77, 57.12, 55.30, 49.48, 33.98, 33.96, 23.74, 21.85. HRMS (C₂₅H₂₇ClN₄O₂+H)⁺ Calc'd: 451.1901, Found: 451.1911.

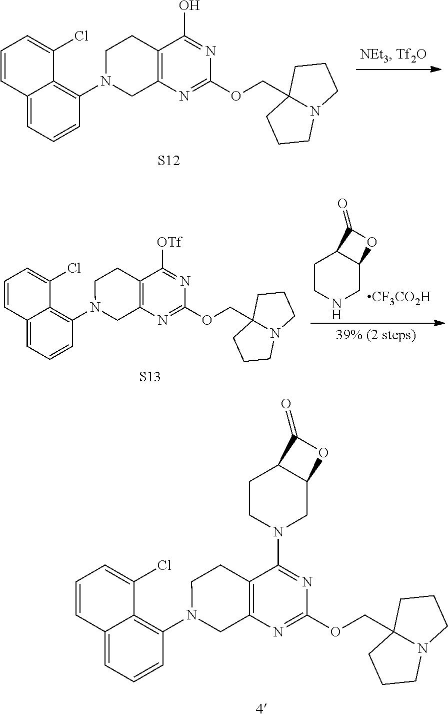

β-lactone 4′, also referred to herein as 13-019.

Triethylamine (29.5 μL, 0.212 mmol, 3.00 equiv.) and triflic anhydride (14.2 μL, 0.0850 mmol, 1.20 equiv.) were added sequentially to a stirred solution of S12 (30.0 mg, 0.0710 mmol, 1 equiv.) in dichloromethane (0.18 mL) at 0° C. In 15 min, TLC analysis (10% methanol-dichloromethane) showed full conversion of the starting material to a slightly less polar spot. The reaction mixture was partitioned between saturated aqueous sodium bicarbonate solution (5 mL) and dichloromethane (5 mL). The layers were separated, and the aqueous layer was extracted with dichloromethane (2×5 mL). The combined organic layers were dried over sodium sulfate. The dried solution was filtered, and the filtrate was concentrated to afford the triflate as a crude product. DMF (0.350 mL) was added to the residue. (1R,6R)-7-oxa-4-azabicyclo[4.2.0]octan-8-one trifluoroacetic acid salt (34.0 mg, 0.141 mmol, 2.00 equiv.) was added to the resulting solution as a solid. Triethylamine (29.5 μL, 0.212 mmol, 3.00 equiv.) was added via syringe. The resulting brown mixture was stirred at 23° C. for 1 h. The residue was diluted with 50% acetonitrile-water to a volume of 2.0 mL, and the solution was filtered through a 0.45 μM PTFE syringe filter. The filtrate was loaded onto a Redi-Sep C18 30g Gold column equilibrated with 10% acetonitrile-water+0.1% formic acid, and the product was purified by column chromatography (10-100% acetonitrile-water+0.1% formic acid). The product-containing fractions were lyophilized to afford the product as a white solid (14.7 mg, 39.0%).

¹H NMR (formate salt, 400 MHz, MeOD) δ7.83 (ddd, J=8.2, 3.2, 1.2 Hz, 1H), 7.68 (td, J=8.3, 1.2 Hz, 1H), 7.57-7.42 (m, 2H), 7.42-7.24 (m, 2H), 4.98-4.91 (m, 1H), 4.50-4.24 (m, 4H), 4.06-3.96 (m, 1H), 3.93-3.75 (m, 1H), 3.75-3.46 (m, 3H), 3.47-3.37 (m, 2H), 3.26-2.90 (m, 4H), 2.86-2.56 (m, 2H), 2.41-1.84 (m, 10H). ¹³C NMR (formate salt, 100 MHz, MeOD) δ 171.40, 168.86, 166.52, 164.39, 161.57, 148.47, 137.49, 129.51, 129.37, 128.17, 126.42, 125.71, 125.37, 124.96, 118.57, 107.77, 76.95, 70.06, 69.83, 59.34, 55.17, 49.83, 46.17, 44.00, 43.81, 34.76, 26.87, 25.74, 24.08, 19.51, 18.99. Note: Product appears as a mixture of conformational isomers. NMR peaks were reported as seen. HRMS (C₃₁H₃₄ClN₅O₃+H)⁺ Calc'd: 560.2428, Found: 560.2427.

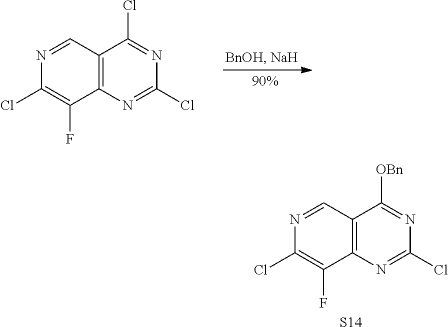 [SMILES: Fc1c(Cl)ncc2c(Cl)nc(Cl)nc12 ; Fc1c(Cl)ncc2c(OCc3ccccc3)nc(Cl)nc12 ; "S14"]

### 4-(benzyloxy)-2,7-dichloro-8-fluoropyrido[4,3-d]pyrimidine (S14)

An oven-dried vial was charged with sodium hydride (60% dispersion in mineral oil, 1.0 g, 25.0 mmol), THF (12.5 mL), and a magnetic stir bar. A solution of benzyl alcohol (2.5 mL in 12.5 mL THF) was added dropwise at 23° C. In 10 min, gas evolution had subsided. The resulting cloudy mixture was allowed to settle for −30 min, and the supernatant was used as a 1.0 M BnONa solution in THF. In a separate 100-mL flask, 2,4,7-trichloro-8-fluoro-pyrido[4,3-d]pyrimidine (2.50 g, 9.90 mmol, 1 equiv) was dissolved in THF (7.6 mL), and the resulting solution was cooled to 0° C. BnONa (1.0 M in THF, 17.0 mL, 1.70 equiv) was added via syringe, and the resulting mixture was stirred for 30 min at 0° C. The reaction mixture was partitioned between saturated aqueous sodium bicarbonate solution (50 mL) and ethyl acetate (50 mL). The layers were separated, and the aqueous layer was extracted with ethyl acetate (2×50 mL). The combined organic layers were dried over sodium sulfate. The dried solution was filtered, and the filtrate was concentrated. The residue was purified by column chromatography (0-30% ethyl acetate-hexanes, 80-g RediSep(R) Rf column, Teledyne ISCO, Lincoln, NE) to afford the product as a white solid (2.90 g, 90%).

¹H NMR (400 MHz, CDCl₃) δ 9.10 (s, 1H), 7.57-7.49 (m, 2H), 7.49-7.39 (m, 3H), 5.72 (s, 2H). ¹³C NMR (100 MHz, CDCl₃) δ 167.93 (d, J=2 Hz), 161.85, 148.20 (d, J=270 Hz), 146.38 (d, J=12 Hz), 143.64 (d, J=8 Hz), 141.06 (d, J=16 Hz), 133.89, 129.26, 129.03, 128.91, 111.70, 71.25. ¹⁹F NMR (376 MHz, CDCl₃) δ −131.58. HRMS (C₁₄H₈Cl₂FN₃O+H)⁺ Calc'd: 324.0107, Found: 324.0267.

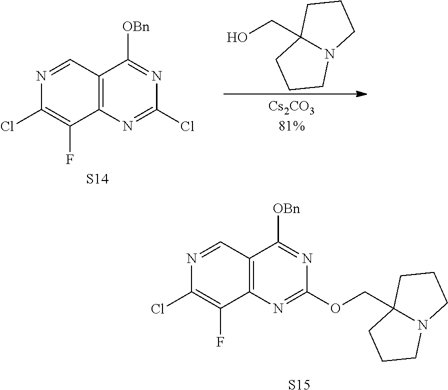 [SMILES: Fc1c(Cl)ncc2c(OCc3ccccc3)nc(Cl)nc12 ; OCC12CCCN1CCC2 ; "S14" ; Fc1c(Cl)ncc2c(OCc3ccccc3)nc(OCC34CCCN3CCC4)nc12 ; "S15"]

### 4-(benzyloxy)-7-chloro-8-fluoro-2-((tetrahydro-1H-pyrrolizin-7a(5H)-yl)methoxy)pyrido[4,3-d]pyrimidine (S15)

An oven-dried 20-mL vial was charged with 4-benzyloxy-2,7-dichloro-8-fluoro-pyrido[4,3-d]pyrimidine (2.50 g, 7.71 mmol, 1 equiv), cesium carbonate (6.28 g, 19.3 mmol, 2.50 equiv), and a magnetic stir bar. 1,4-Dioxane (15.4 mL) and 1,2,3,5,6,7-hexahydropyrrolizin-8-ylmethanol (1.63 g, 11.6 mmol, 1.50 equiv) were added sequentially via syringe. The resulting mixture was heated to 60° C. for 3 h. The reaction mixture was filtered through a 10-micron polyethylene funnel, and the filtrate and concentrated under reduced pressure. The residue was purified by column chromatography (0-20% methanol-dichloromethane+0.2% ammonium hydroxide, 80-g RediSep(R) Rf column, Teledyne ISCO, Lincoln, NE) to afford the product as a white solid (2.69 g, 81.3%).

¹H NMR (400 MHz, CDCl₃) δ 8.93 (s, 1H), 7.53-7.47 (m, 2H), 7.47-7.36 (m, 3H), 5.65 (s, 2H), 4.33 (s, 2H), 3.25-3.07 (m, 2H), 2.67 (dt, J=10.2, 6.8 Hz, 2H), 2.06 (dt, J=12.4, 6.1 Hz, 2H), 1.90 (p, J=6.5 Hz, 4H), 1.69 (dt, J=12.5, 7.5 Hz, 2H). ¹³C NMR (100 MHz, CDCl₃) δ 169.03, 165.23, 149.63, 147.78, 147.67, 146.96, 143.12, 143.04, 140.07, 134.74, 128.85, 128.79, 128.56, 111.15, 74.30, 72.29, 70.05, 55.69, 36.04, 25.58. ¹⁹F NMR (376 MHz, CDCl₃) δ −134.41. HRMS (C₂₂H₂₂ClFN₄O₂+H)⁺ Calc'd: 429.1494, Found: 429.1590.

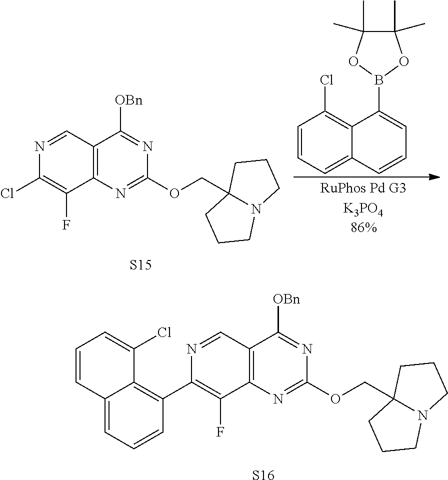

### 4-benzyloxy-7-(8-chloro-1-naphthyl)-8-fluoro-2-(1,2,3,5,6,7-hexahydropyrrolizin-8-ylmethoxy)pyrido[4,3-d]pyrimidine (S16)

A 100-mL flask was charged with 4-benzyloxy-7-chloro-8-fluoro-2-(1,2,3,5,6,7-hexahydropyrrolizin-8-ylmethoxy)pyrido[4,3-d]pyrimidine (1.50 g, 3.50 mmol, 1 equiv.), 2-(8-chloro-1-naphthyl)-4,4,5,5-tetramethyl-1,3,2-dioxaborolane (1.51 g, 5.25 mmol, 1.50 equiv.) and a magnetic stir bar. Toluene (5.8 mL) and an aqueous solution of potassium phosphate (1.50 M, 7.0 mL, 10.5 mmol, 3.00 equiv.) was added sequentially, giving rise to a biphasic mixture. A stream of argon was bubbled through the mixture for 5 min via a 20G needle. RuPhos Pd G3 (585 mg, 0.700 mmol, 0.200 equiv.) was added in one portion, the flask was fitted with a rubber septum, and the reaction mixture was heated to 60° C. for 18 h. The reaction mixture was cooled to 23° C. and then partitioned between water (20 mL) and ethyl acetate (20 mL). The layers were separated, and the aqueous layer was extracted with ethyl acetate (2×20 mL). The combined organic layers were washed with saturated sodium chloride solution, and the washed solution was dried over sodium sulfate. The dried solution was filtered, and the filtrate was concentrated. The residue was purified by column chromatography (0-30% methanol-dichloromethane, 40-g RediSep(R) Rf column, Teledyne ISCO, Lincoln, NE) to afford the product as a yellow powder (1.67 g, 86%).

¹H NMR (400 MHz, CDCl₃) δ 9.23 (s, 1H), 8.02 (dd, J=8.0, 1.6 Hz, 1H), 7.89 (dd, J=8.2, 1.3 Hz, 1H), 7.67-7.52 (m, 6H), 7.51-7.37 (m, 5H), 5.74 (s, 2H), 4.41 (s, 2H), 3.23 (dt, J=11.0, 5.7 Hz, 2H), 2.71 (dt, J=10.3, 6.8 Hz, 2H), 2.29-2.05 (m, 2H), 2.05-1.86 (m, 4H), 1.80-1.68 (m, 2H). ¹³C NMR (100 MHz, CDCl₃) δ 169.20, 164.87, 151.39, 149.83, 149.69, 147.83, 146.79, 142.60, 142.53, 135.93, 135.14, 131.76, 130.98, 130.62, 130.58, 129.38, 128.80, 128.76, 128.69, 128.56, 128.52, 128.35, 126.00, 125.56, 111.03, 73.95, 72.48, 69.75, 55.69, 55.67, 36.06, 36.04, 25.56, 25.54. ¹⁹F NMR (376 MHz, CDCl₃) δ −138.14. HRMS (C₃₂H₂₈ClFN₄O₂+H)⁺ Calc'd: 555.1963, Found: 555.1953.

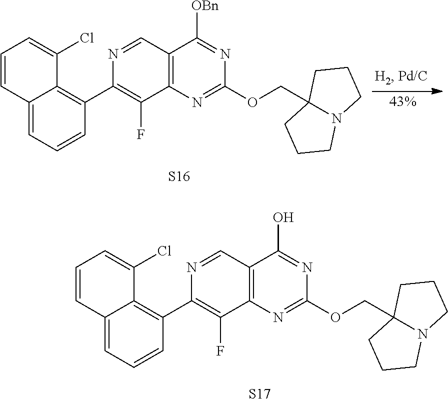 [SMILES: Fc1c(-c2cccc3cccc(Cl)c23)ncc2c(OCc3ccccc3)nc(OCC34CCCN3CCC4)nc12 ; "S16" ; Oc1nc(OCC23CCCN2CCC3)nc2c(F)c(-c3cccc4cccc(Cl)c34)ncc12 ; "S17"]

### 7-(8-chloro-1-naphthyl)-8-fluoro-2-(1,2,3,5,6,7-hexahydropyrrolizin-8-ylmethoxy)pyrido[4,3-d]pyrimidin-4-ol (S17)

Argon was bubbled through a solution of 4-benzyloxy-7-(8-chloro-1-naphthyl)-8-fluoro-2-(1,2,3,5,6,7-hexahydropyrrolizin-8-ylmethoxy)pyrido[4,3-d]pyrimidine (2.50 g, 4.50 mmol, 1 equiv) in methanol (45 mL) for 5 min via a 20G needle. Palladium on carbon (10% wt, 479 mg, 0.450 mmol, 0.10 equiv.) was added carefully under a blanket of argon. The vial was fitted with a rubber septum, and a stream of hydrogen (balloon) was bubbled through the reaction mixture through a 22G×4″ needle. In 1 h, LC-MS analysis indicated full consumption of the starting material. The gas source was switched to argon and bubbling was continued for 5 min. The reaction mixture was filtered through a tightly packed pad of Celite under the protection of argon, and the filter cake was rinsed with methanol (2×10 mL). Care was taken not to allow the filter cake to become dry, and the filter cake was immediately moisturized with water upon completion of the filtration. The combined filtrate was concentrated under reduced pressure. The residue was purified by column chromatography (0-30% methanol-dichloromethane, 4-g RediSep(R) Rf column, Teledyne ISCO, Lincoln, NE) to afford the product as a yellow powder (890 mg, 42.5%).

¹H NMR (400 MHz, CDCl₃) δ 9.21 (s, 1H), 7.99 (dd, J=6.7, 2.8 Hz, 1H), 7.87 (dd, J=8.2, 1.3 Hz, 1H), 7.64-7.52 (m, 3H), 7.41 (t, J=7.8 Hz, 1H), 4.47 (app q, J=12.7 Hz, 2H), 3.64-3.49 (m, 2H), 3.04-2.89 (m, 2H), 2.29-2.03 (m, 6H), 1.98-1.84 (m, 2H). ¹⁹F NMR (376 MHz, CDCl₃) δ −139.61. ¹³C NMR (100 MHz, CDCl₃) δ 168.68, 162.34, 151.65, 149.89, 149.74, 149.08, 145.20, 145.09, 144.21, 144.15, 135.83, 132.22, 132.19, 131.00, 130.72, 130.17, 129.21, 128.69, 128.23, 125.77, 125.64, 116.97, 71.29, 55.51, 54.93, 35.19, 34.69, 24.90, 24.62. HRMS (C₂₅H₂₂ClFN₄O₂+H)⁺ Calc'd: 465.1494, Found: 465.1518.

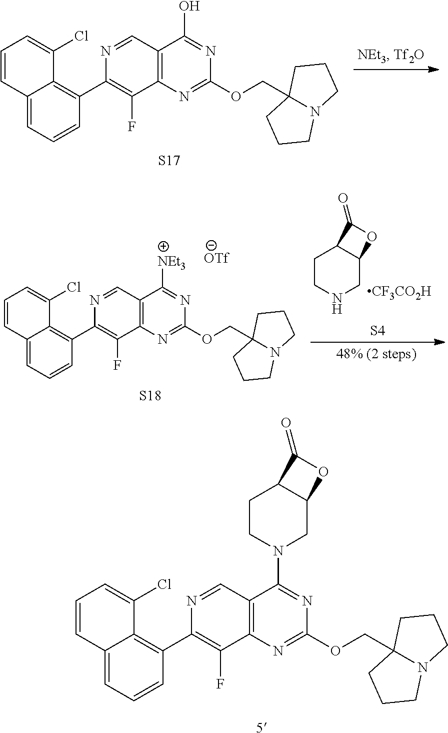

β-lactone 5′, also referred to herein as 13-036.

A solution of S17 (50.0 mg, 0.107 mmol, 1 equiv.) in dichloromethane (0.27 mL) was cooled to 0° C., and triethylamine (45.0 μL, 0.323 mmol, 3.00 equiv.) and triflic anhydride (36.1 μL, 0.215 mmol, 2.00 equiv.) were added sequentially via syringe. In 1 h at 0° C., LC-MS analysis indicated that the starting material had been fully converted to the trimethylamine adduct. The reaction mixture was directly concentrated under reduced pressure. DMF (0.27 mL) and triethylamine (45.0 μL, 0.323 mmol, 3.00 equiv.) were added to the residue, giving rise to a brown solution. (1R,6R)-8-oxa-3-azabicyclo[4.2.0]octan-7-one trifluoroacetate salt (25.9 mg, 0.107 mmol, 2.00 equiv.) was added as a solid. After stirring at 23° C. for 1 h, the reaction mixture was diluted with 2.0 mL 1:1 acetonitrile:water+5% formic acid. The resulting solution was loaded onto reverse-phase C18 column (Teledyne ISCO RediSep C18 30g Gold). Elution was performed with a linear gradient of 10-95% acetonitrile-water+0.1% formic acid, and the product-containing fractions were lyophilized to afford the product as a yellow powder (31.0 mg, 48.3%).

¹H NMR (formate salt, 400 MHz, MeOD) δ 8.17 (dd, J=8.2, 1.4 Hz, 1H), 8.04 (dd, J=8.2, 1.3 Hz, 1H), 7.75-7.61 (m, 3H), 7.57-7.48 (m, 1H), 5.18-5.08 (m, 1H), 4.93 (dtd, J=15.7, 6.3, 2.3 Hz, 1H), 4.69 (s, 2H), 4.36 (q, J=7.1 Hz, 1H), 4.28-4.08 (m, 3H), 3.76-3.67 (m, 2H), 3.33-3.25 (m, 2H), 2.42-2.30 (m, 4H), 2.30-2.03 (m, 5H), 1.43-1.29 (m, 2H). ¹⁹F NMR (formate salt, 376 MHz, MeOD) δ −139.57, −139.90. ¹³C NMR (formate salt, 100 MHz, MeOD) δ 170.17, 164.48, 163.18, 144.08, 144.01, 136.00, 135.95, 130.90, 130.76, 129.88, 129.26, 128.47, 128.24, 126.08, 125.48, 125.43, 121.77, 118.83, 111.03, 80.32, 69.59, 68.97, 55.26, 46.42, 45.91, 45.69, 34.00, 23.86, 19.63, 19.54, 7.59. Note: Product appears as a mixture of conformational isomers. NMR peaks were reported as seen. HRMS (C₃₁H₂₉ClFN₅O₃+H)⁺ Calc'd: 574.2021, Found: 574.2015.

### REFERENCES FOR EXAMPLE 2

1. Hunter, J. C.; Manandhar, A.; Carrasco, M. A.; Gurbani, D.; Gondi, S.; Westover, K. D. Mol. Cancer Res. 2015, 1325-1336. 2. Ostrem, J. M.; Peters, U.; Sos, M. L.; Wells, J. A.; Shokat, K. M. Nature 2013, 503 (7477), 548-551. 3. Ahmadian, M. R.; Zor, T.; Vogt, D.; Kabsch, W.; Selinger, Z.; Wittinghofer, A; Scheffzek, K. Proc. Natl. Acad. Sci. U.S.A. 1999, 96 (12), 7065-7070. 4. Huehls, A. M.; Coupet, T. A.; Sentman, C. L. Immunol. Cell Biol. 2014, 93 (10), 290-296. 5. Maurer, T.; Garrenton, L. S.; Oh, A.; Pitts, K.; Anderson, D. J.; Skelton, N. J.; Fauber, B. P.; Pan, B.; Malek, S.; Stokoe, D.; Ludlam, M. J. C.; Bowman, K. K.; Wu, J.; Giannetti, A. M.; Starovasnik, M. A.; Mellman, I.; Jackson, P. K.; Rudolph, J.; Wang, W.; Fang, G. Proc. Natl. Acad. Sci. U.S.A. 2012, 109 (14), 5299-5304. 6. Gibson, D. G.; Young, L.; Chuang, R. Y.; Venter, J. C.; Hutchison, C. A.; Smith, H. O. Nat. Methods 2009, 6 (5), 343-345. 7. Gentile, D. R.; Rathinaswamy, M. K.; Jenkins, M. L.; Moss, S. M.; Siempelkamp, B. D.; Renslo, A. R.; Burke, J. E.; Shokat, K. M. Cell Chem. Biol. 2017, 24 (12), 1455-1466.e14. 8. Sondermann, H.; Soisson, S. M.; Boykevisch, S.; Yang, S. S.; Bar-Sagi, D.; Kuriyan, J. Cell 2004, 119 (3), 393-405.

### Example 3: Biological Data

TABLE 3
Data represents the percentage of covalent
modification of test proteins after 24-h incubation with
100 μM compound at 23° C. at pH 7.5.

| | % covalent | % covalent |
|---|---|---|
| | modification of | modification of |
| Compound | wild type K-Ras | K-Ras G12S |
| 04-005 | 0% | 0% |
| 06-092 | 8.0% | 0% |
| 06-093 | 4.8% | 0% |
| 08-043 | 0% | n/a |
| 08-044 | 0% | n/a |
| 12-026 | 7.0% | 100% |
| 12-027 | 0% | 23.0% |
| 12-028 | 0% | 19.0% |
| 12-044 | 0% | 0% |
| 12-058 | 6.0% | 94.0% |
| 12-064 | 0% | 0% |
| 12-074 | 0% | 0% |
| 12-077 | 7.5% | 100% |
| 12-078 | n/a | n/a |
| 12-087 | 0% | 0% |
| 13-005 | 73.7% | 72.1% |
| 13-006 | 0% | 0% |
| 13-018 | 85.8% | 92.8% |
| 13-019 | 0% | 100% |
| 13-036 | 0% | 100% |
| 13-037 | 0% | 42.5% |
| 14-005 | 0% | 100% |
| 14-036 | 22.0% | 42.0% |

### Example 4: Chemical Alkylation of Asp12 Enables Mutant Selective Targeting of Ras(G12D)

Disclosed herein, inter alia, is a set of ligands that alkylate the mutant aspartate in K-Ras(G12D) in both GDP- and GTP-bound states and block effector interactions. In embodiments, these ligands are highly specific for the G12D mutant and do not react with wildtype K-Ras in the assays tested.

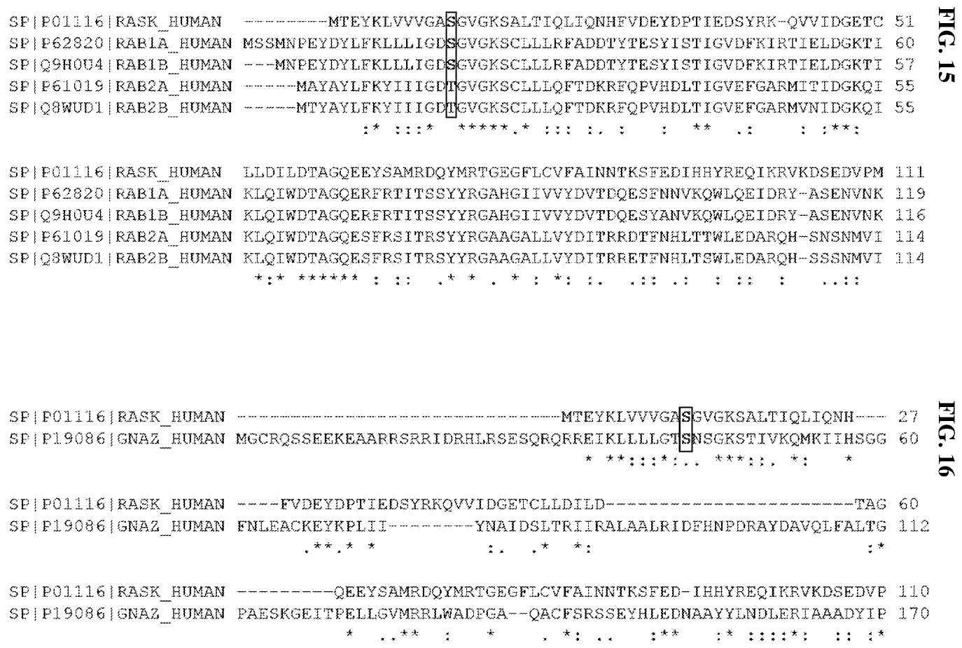

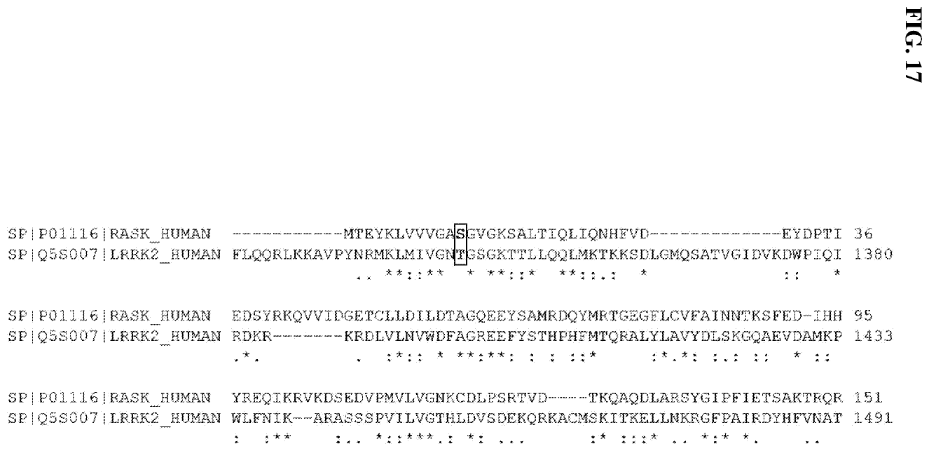

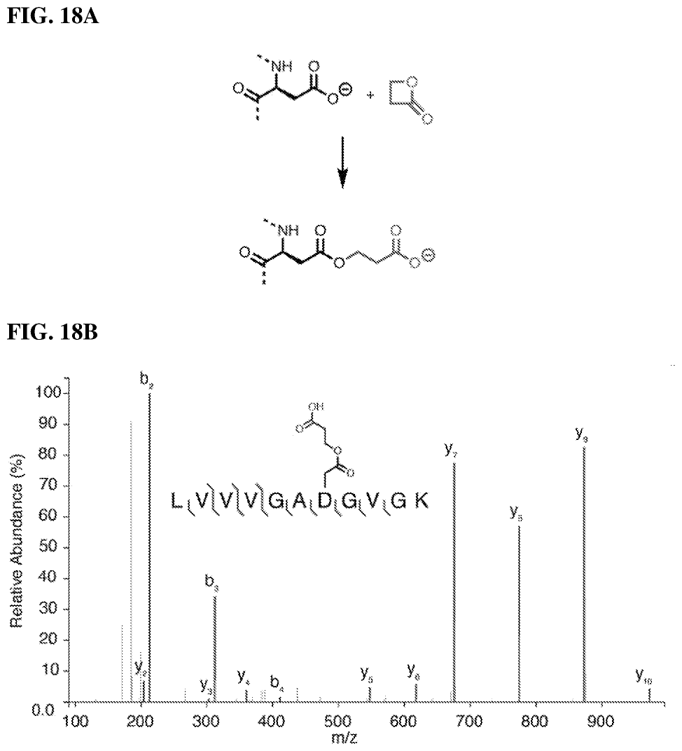

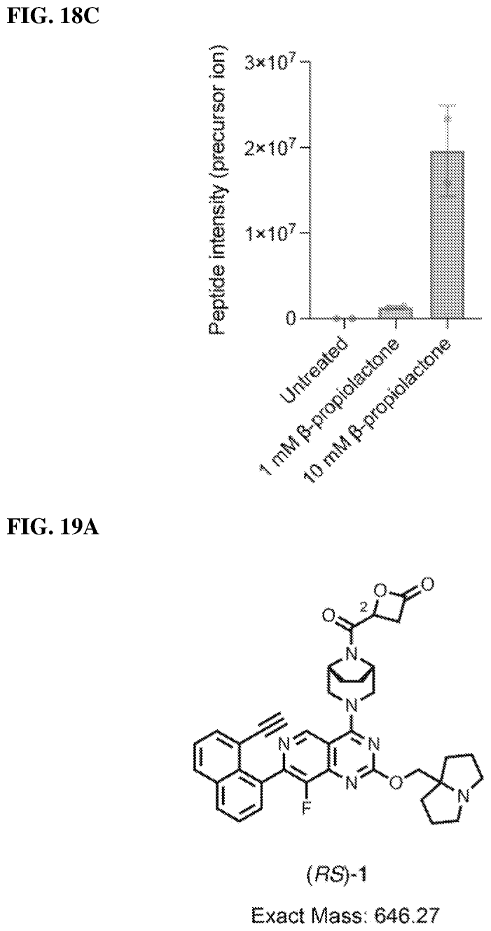

Recombinant K-Ras(G12D)·GDP protein was treated with 1 mM or 10 mM β-propiolactone at 23° C. for 1 h, digested the protein with trypsin, and analyzed the samples by liquid chromatography-tandem mass spectrometry. A range of peptides was detected carrying a β-propiolactone adduct (FIG. 18A). Among these was the peptide LVVVGAD*GVGK (SEQ ID NO:19) (aa. 7-16), where the mutant aspartate was covalently alkylated by a 2-carboxylethyl group (FIG. 18B). The intensity of this peptide increased with the concentration of β-propiolactone (FIG. 18C), suggesting that the site was not saturated under these conditions. These data show that Asp12 is susceptible to alkylation by a β-lactone electrophile.

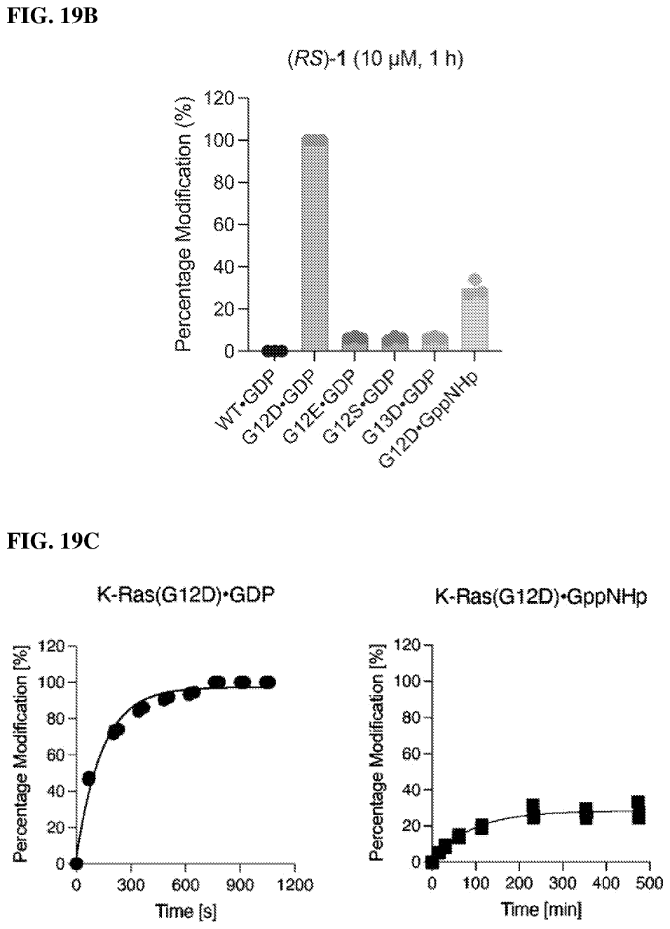

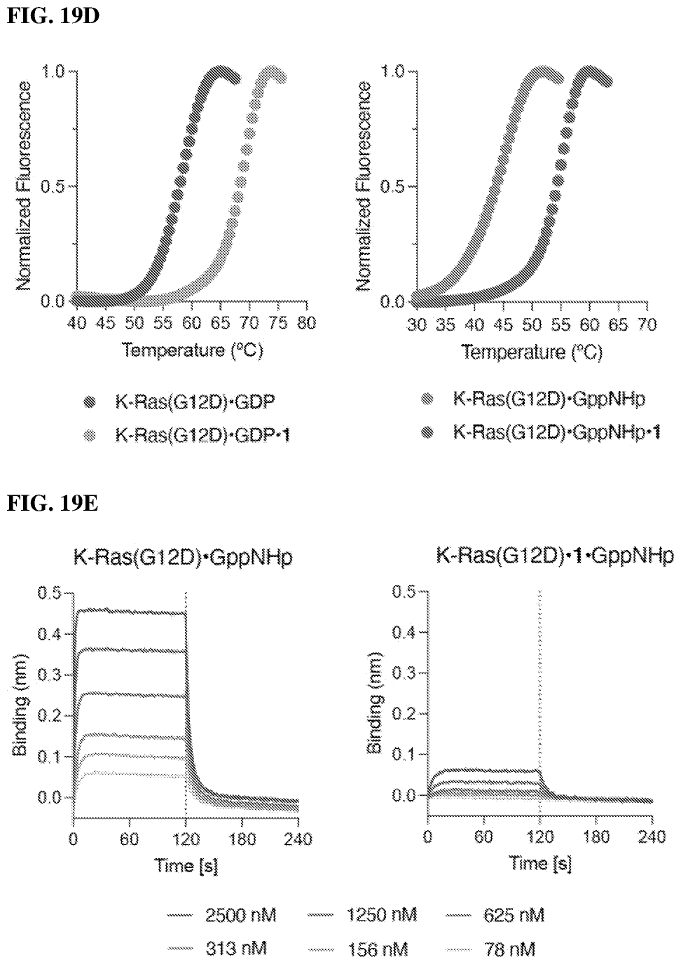

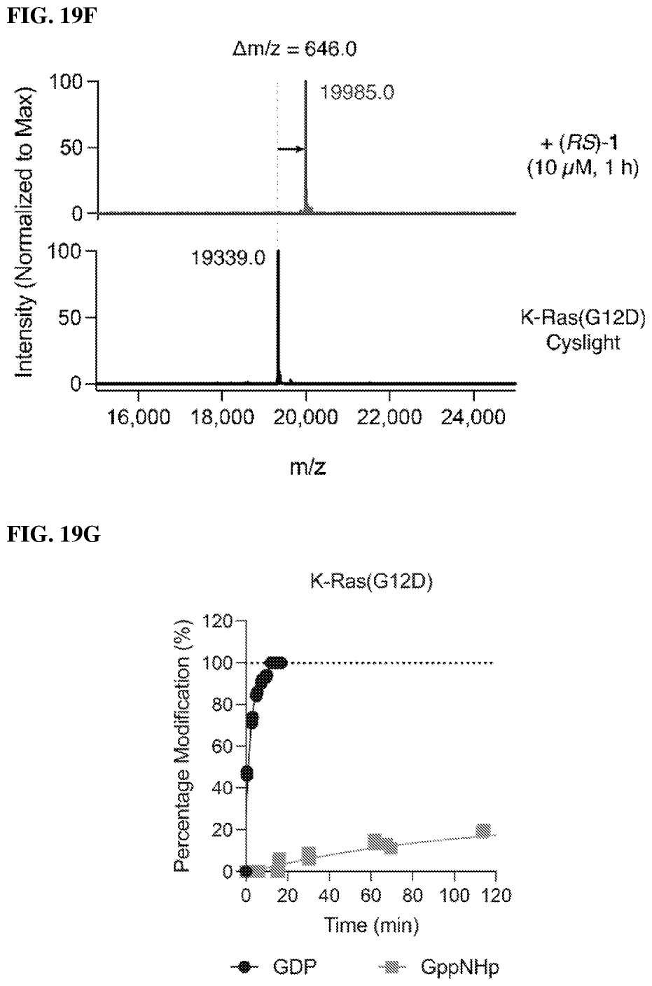

To design chemical compounds that selectively engage K-Ras(G12D), 0-propiolactone was grafted as an electrophilic fragment onto a K-Ras Switch-II pocket (S—IIP) ligand scaffold. The S—IIP ligands occupy an allosteric pocket directly adjacent to the P-loop of K-Ras and provide chemical vectors that allow direct access to the codon 12 mutant residue. Compound 1 was prepared as a racemate by coupling (±)-β-carboxyl-β-propiolactone to the piperazine group of a S—IIP ligand scaffold for pan-K-Ras (FIG. 19A). Using whole protein mass spectrometry, the reactions between recombinant K-Ras proteins and 10 μM Compound 1 were assessed at 23° C. (FIG. 19B, FIG. 19F). Compound 1 reacted rapidly with K-Ras(G12D)·GDP with a half-life of 99 s (95% CI: 83-118 s) and fully modified K-Ras(G12D)·GDP in less than 15 minutes (FIG. 19G). By contrast, it only achieved 6.5±0.3% modification of K-Ras(G13D)·GDP and no detectable modification of K-Ras(wildtype)·GDP after 1 h. Interestingly, Compound 1 showed markedly reduced reactivity with modified K-Ras(G12E)·GDP, a non-natural mutant that positions its carboxylate nucleophile (Glu) further from the backbone than that in G12D (Asp). Compound 1 also did not react with K-Ras(G12S)·GDP, a mutant that was recently targeted with a different 0-lactone electrophile (22). Although less potent, compound 1 was also active against the GppNHp-bound K-Ras(G12D). This is distinct from S—IIP K-Ras(G12C) inhibitors, which are reported to specifically recognize the GDP-bound state. The covalent modification of Asp12 by compound 1 stabilized both K-Ras(G12D)·GDP and K-Ras(G12D)·GppNHp toward thermal denaturation (ΔTm=+10.3° C. and +2.5° C., respectively, FIG. 19D). The adduct between K-Ras(G12D) and compound 1 was stable over a range of pH (5.0-8.5) and did not degrade in the presence of 1 mM DTT at 23° C.

It was further tested whether such a covalent modification could disrupt the interaction with effector proteins such as Raf. Using a time-resolved fluorescence energy transfer assay, it was found that compound 1 inhibited the interaction between Raf-RBD and K-Ras(G12D), but not wildtype K-Ras, in the dose-dependent fashion (FIG. 19H). The interaction between immobilized Raf-RBD with fully labeled K-Ras(G12D)·GppNHp·compound 1 was measured using biolayer interferometry. Compared to unmodified K-Ras(G12D)·GppNHp, the K-Ras(G12D)·GppNHp·compound 1 complex showed substantially decreased binding to Raf-RBD (FIG. 19E), indicating that compound 1 directly impeded Ras-effector interaction in the GTP-state.

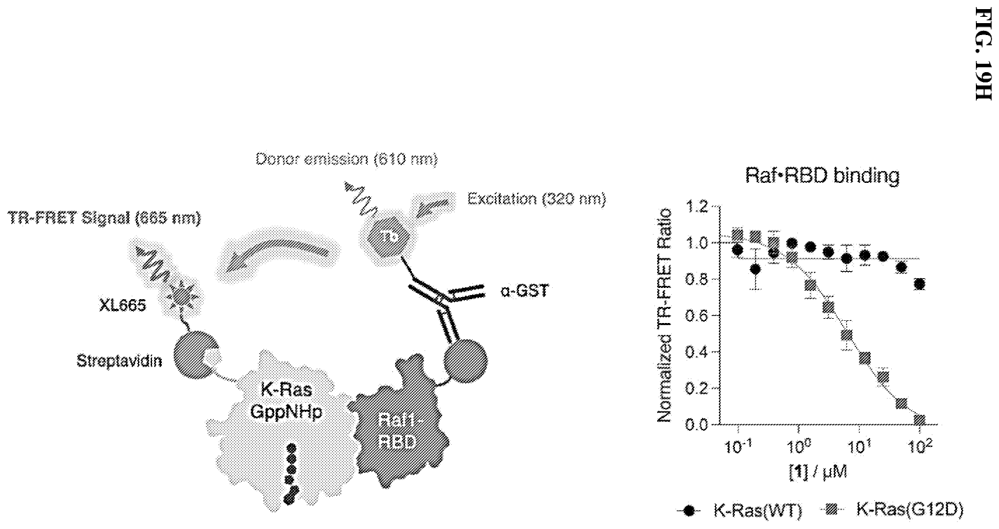

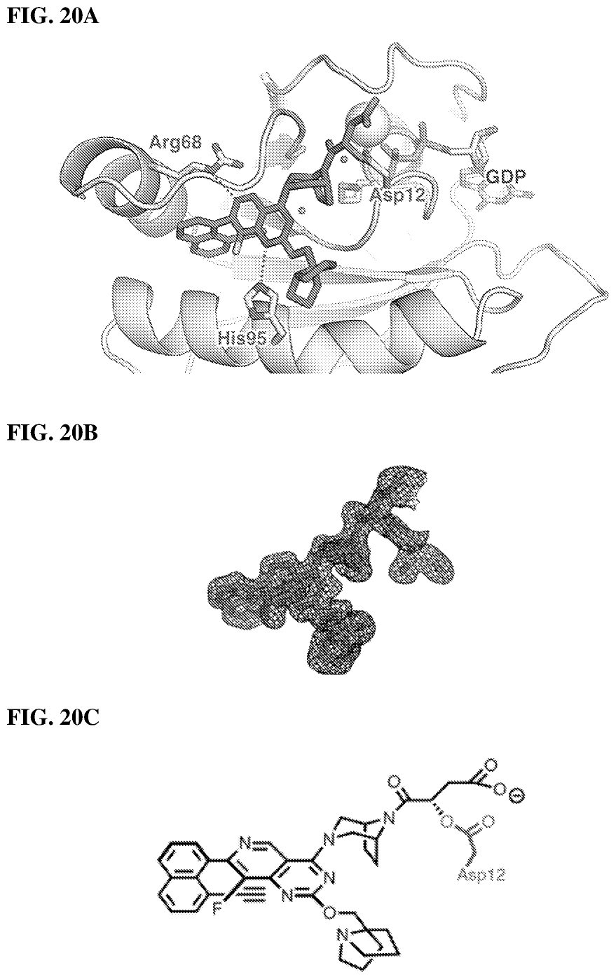

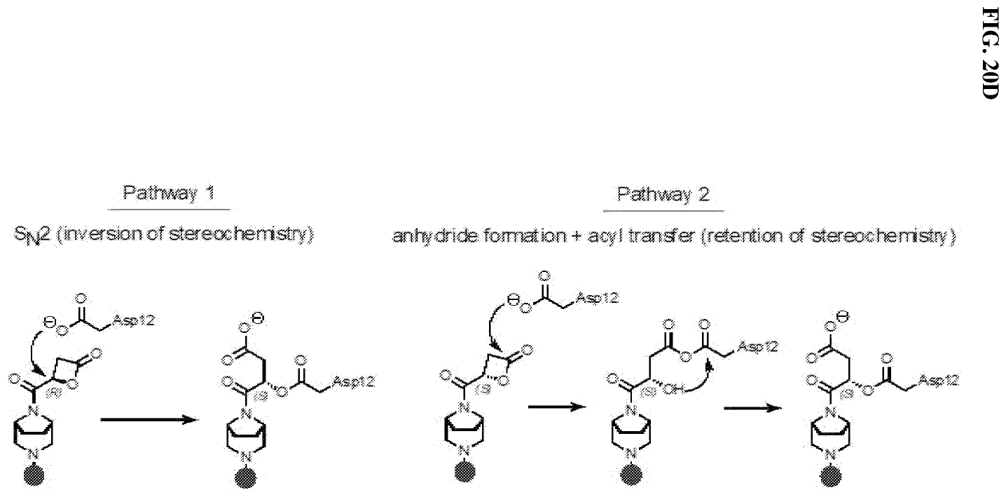

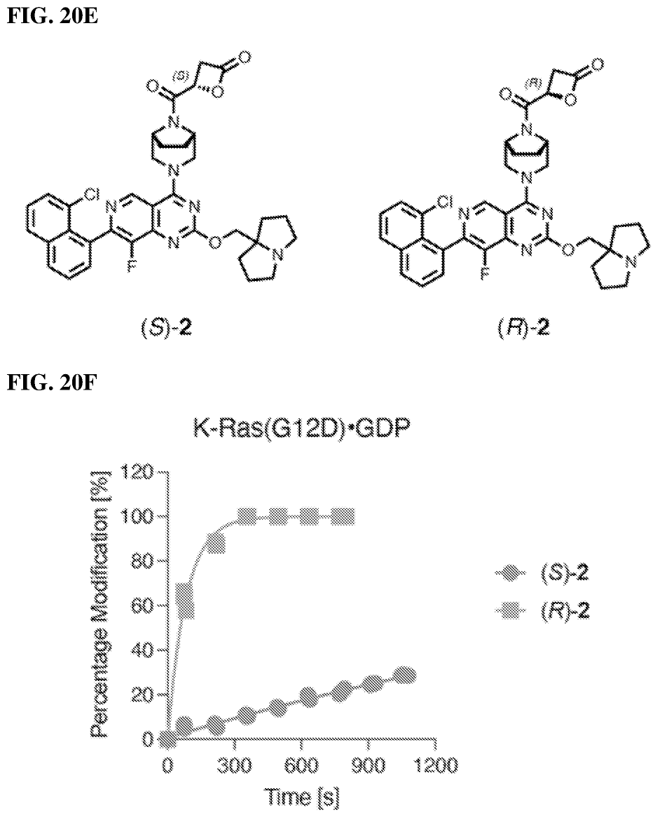

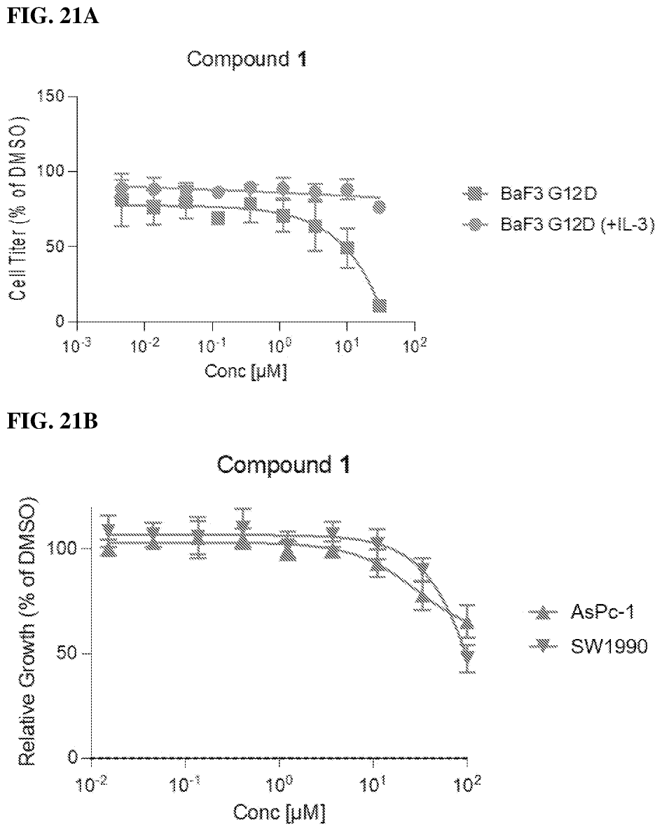

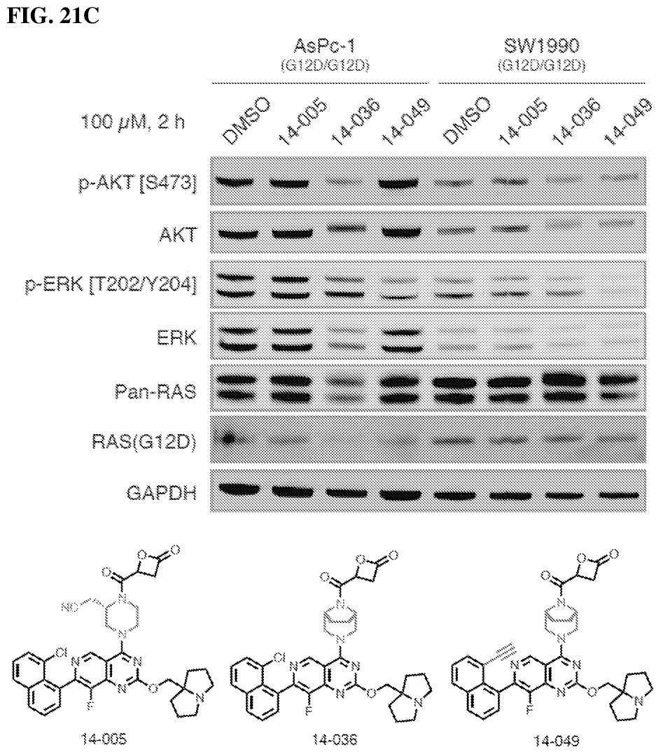

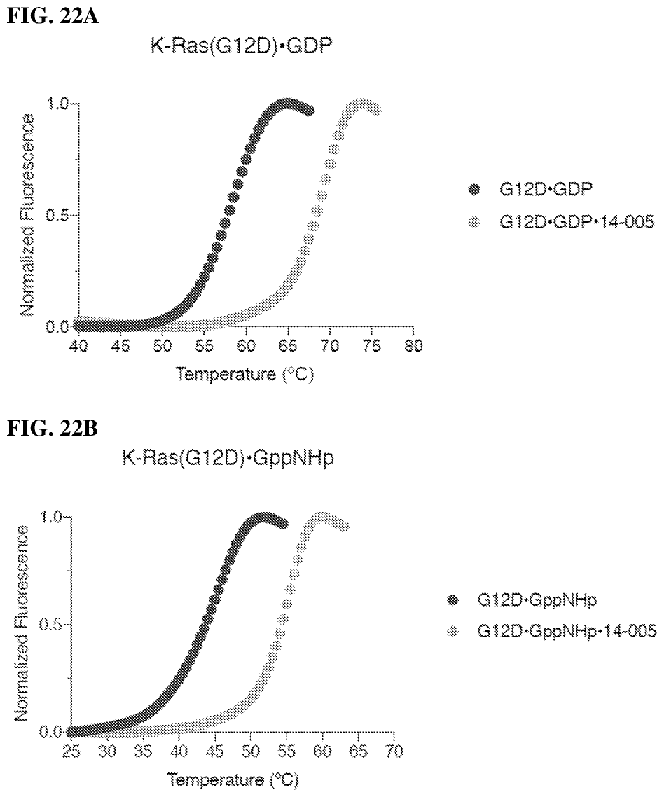

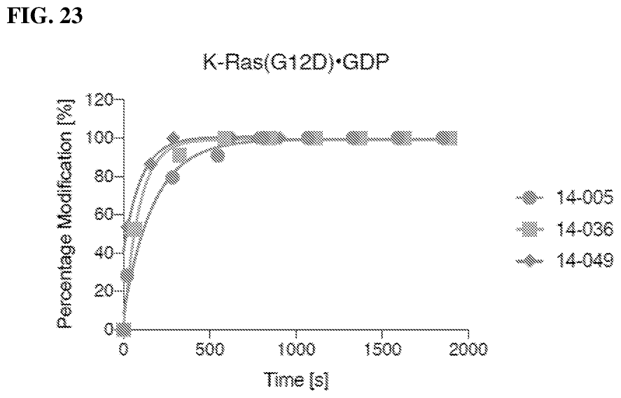

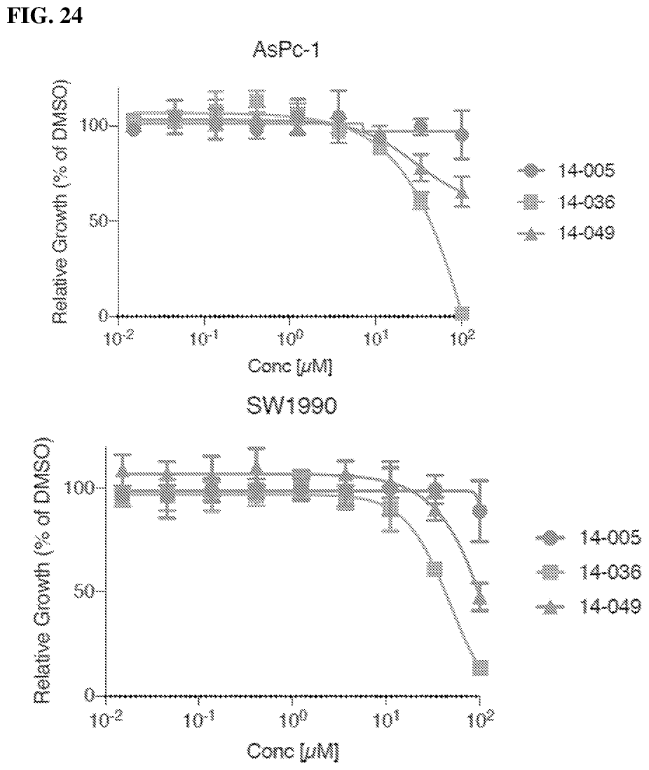

Since β-lactones can act either as an acylating reagent (via nucleophilic attack on the carbonyl group) or as an alkylating reagent (via nucleophilic attack on the β-carbon), the reaction between compound 1 and K-Ras(G12D) could give an anhydride product or an ester product. A 1.7-Å crystal structure of the K-Ras(G12D)·GDP·1 adduct (FIG. 20A) was used to solve the reaction pathway. Compound 1 in the S—IIP pocket was observed of adopting a conformation similar to that seen for K-Ras(G12C) inhibitors, with clear electron density for the covalent bond between Asp12 and the compound as well as the free carboxyl group resulting from the ring opening (FIG. 20B). This high-resolution structure also allowed assigning the stereochemistry of the adduct as S at the R carbon (FIG. 20C). Because the co-crystal was obtained using racemic compound 1, this S stereochemistry could in theory result from (1) a S_{N}2 attack on the β-carbon of R enantiomer of 1, or (2) an attack on the carbonyl of the S enantiomer of 1 followed by an acyl transfer (FIG. 20D). Pure R and S enantiomers of a structural analog 2 were prepared. Monitoring the reaction rate with K-Ras(G12D)·GDP showed that the R enantiomer was more reactive toward K-Ras(G12D)·GDP (FIG. 20F).

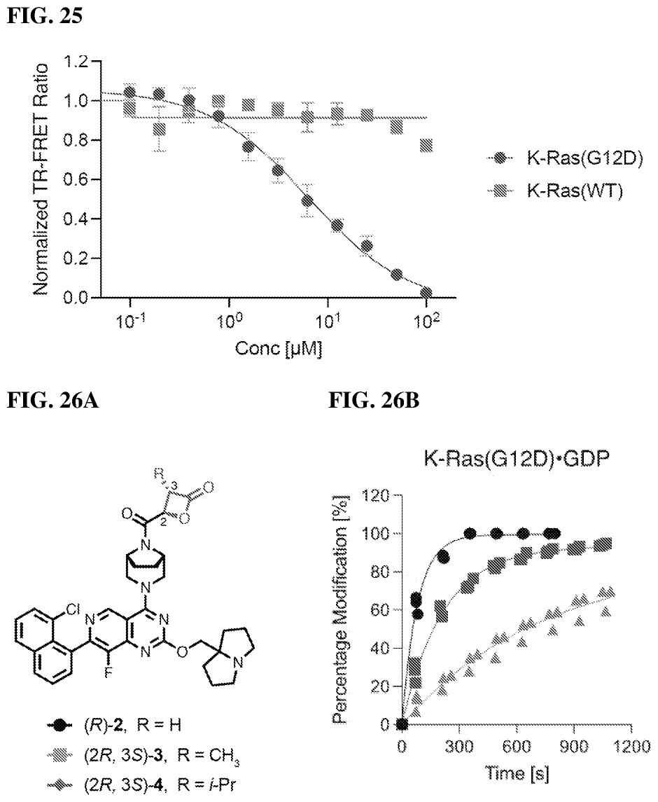

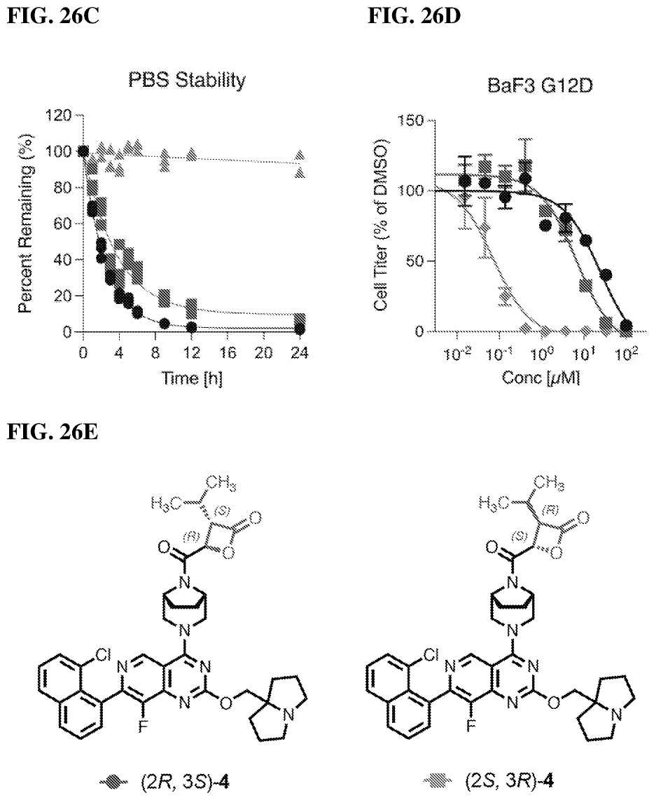

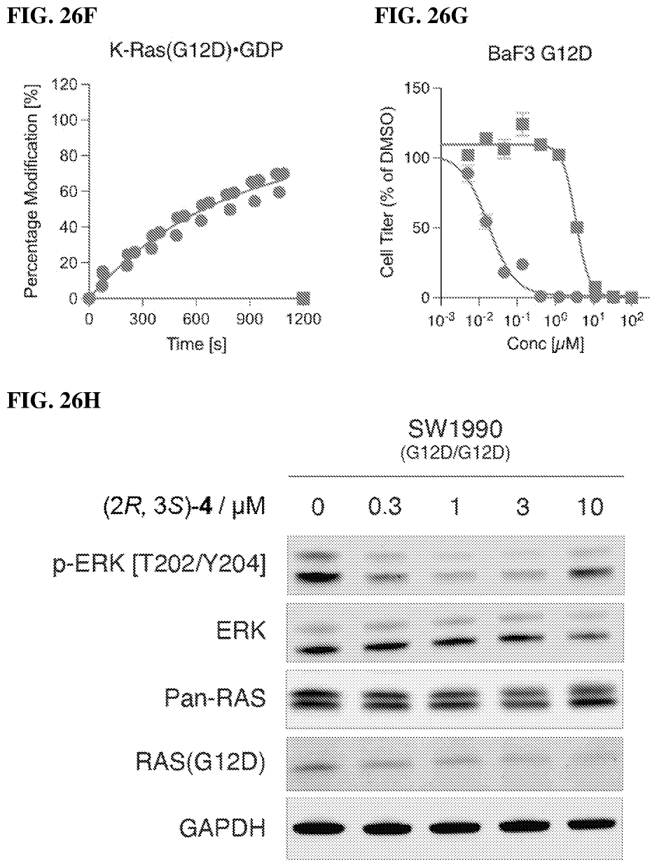

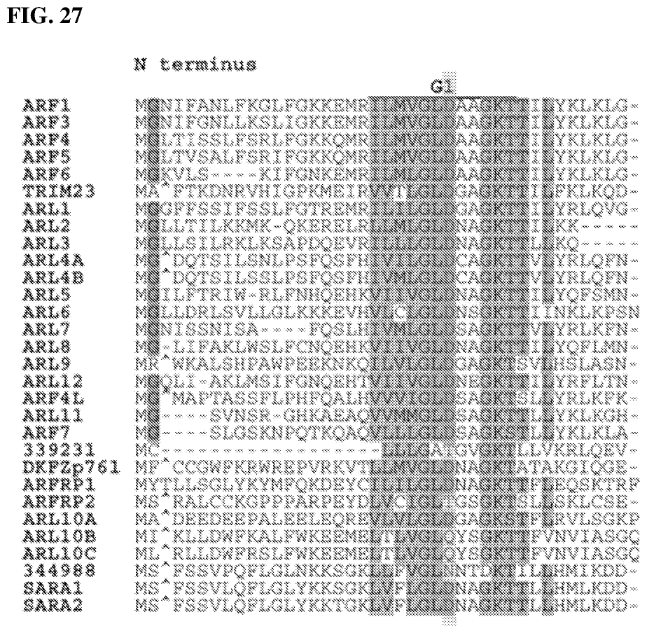

Compounds 2-4 were synthesized with increasing steric hinderance at the pro-S position of the α-carbon (FIG. 26A). Compounds 2-4 maintained the ability to react with K-Ras(G12D), although the bulkier compounds exhibited decreased reaction rates (FIG. 26B). Meanwhile, compound 4, bearing an isopropyl group, resisted hydrolysis, with >90% of the material remaining intact after 24 h at 23° C. and pH 7.4. By contrast, compounds 2 and 3 had half-lives of 1.7 h and 2.5 h, respectively (FIG. 26C). Compound 4 inhibited the growth of BaF3/K-Ras(G12D) cells (an engineered cell line whose proliferation is dependent on KRAS G12D) with an IC₅₀ of 71 nM (95% CI: 48-105 nM) (FIG. 26D). Interestingly, the enantiomer of (2R, 3S)-4 did not show any detectable reactivity with K-Ras(G12D) even after extended incubation, confirming that the 2R, 3S stereochemistry of the β-lactone in compound 4 is essential for the ligand-protein recognition. This enantiomeric compound ((2S, 3R)-4) was also found much less potent at inhibiting BaF3/K-Ras(G12D) proliferation (IC₅₀=4.4 μM, 95% CI: 3.1-6.3 μM). Together, the data suggests that (2R, 3S)-4 is a potent covalent ligand of K-Ras(G12D) with cellular activity.

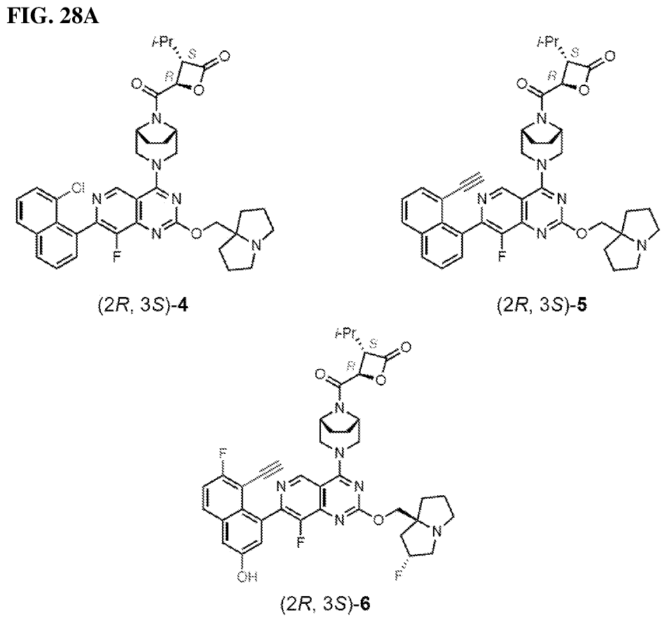

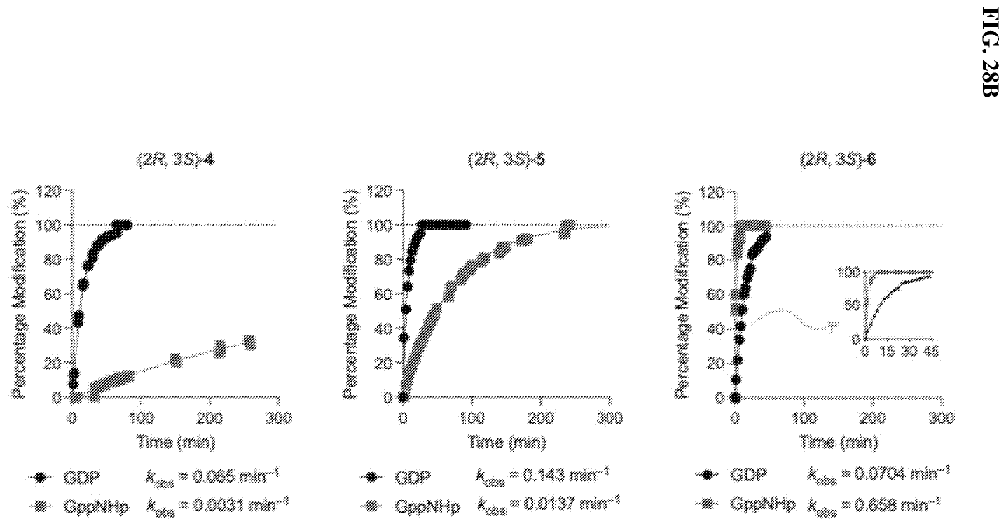

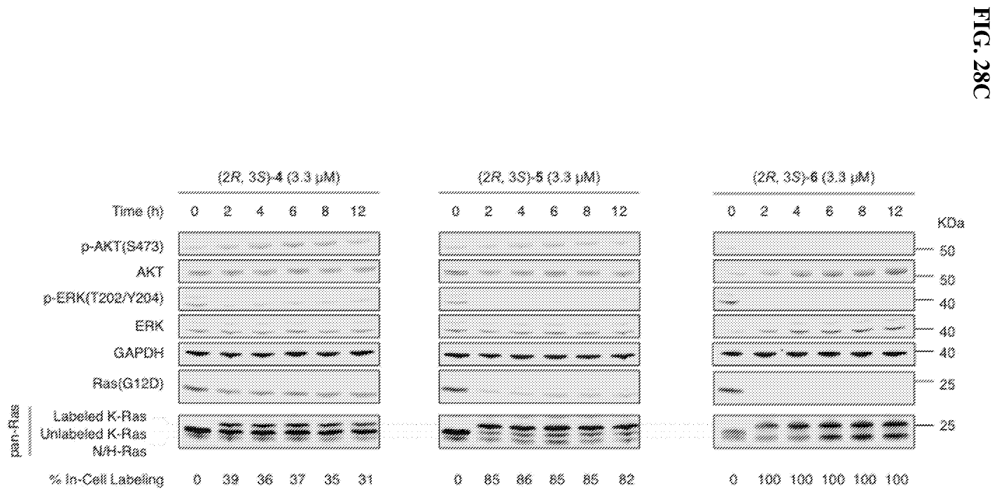

With the fast kinetics of GDP-state labeling persisted, Compounds 5 and 6 labeled the GTP-state up to 200-fold faster than compound 4. When tested at 10 PM, Compound 6 labeled K-Ras(G12D)·GppNHp to completeness within 5 min. The in-cell K-Ras(G12D) covalent labeling kinetics by Western blot time course mirrored the recombinant protein results (FIG. 28C), where Compound 6 labeled endogenous K-Ras(G12D) completely in homozygous KRAS^{G12D/G12D} cancer cell line SW1990 within 2 h as indicated by the gel mobility shift in the anti-Ras immunoblot, and concomitant reduction of the phospho-ERK levels. This is consistent with its higher potency observed with K-Ras(G12D)·GppNHp, as cellular K-Ras(G12D) is enriched in the GTP-bound state as a result of its poor GTPase activity (23). The lack of effect of both compounds on phospho-AKT levels agrees with previous reports that K-Ras(G12D) does not induce Akt phosphorylation (24).

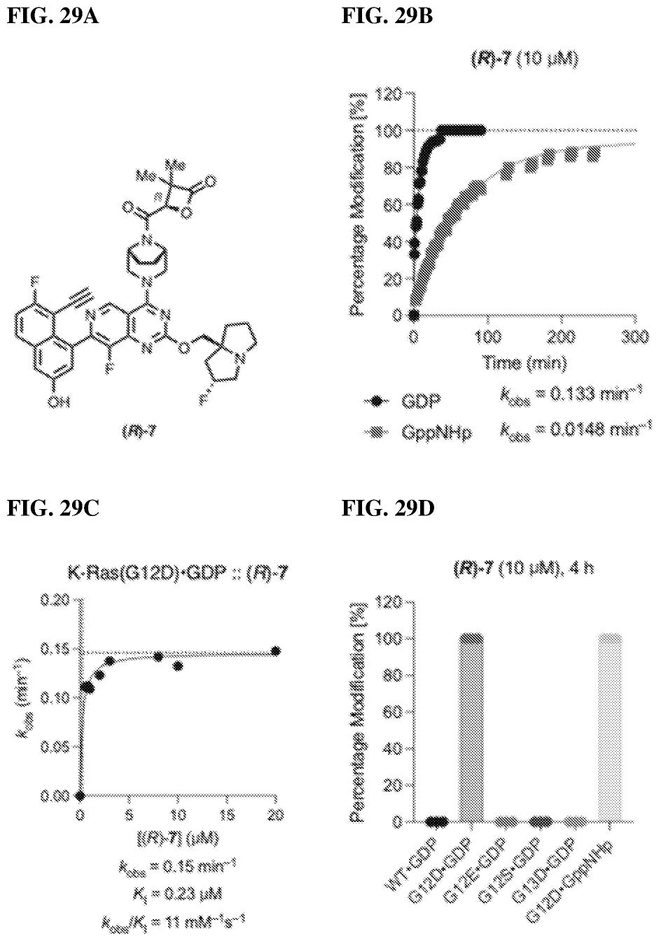

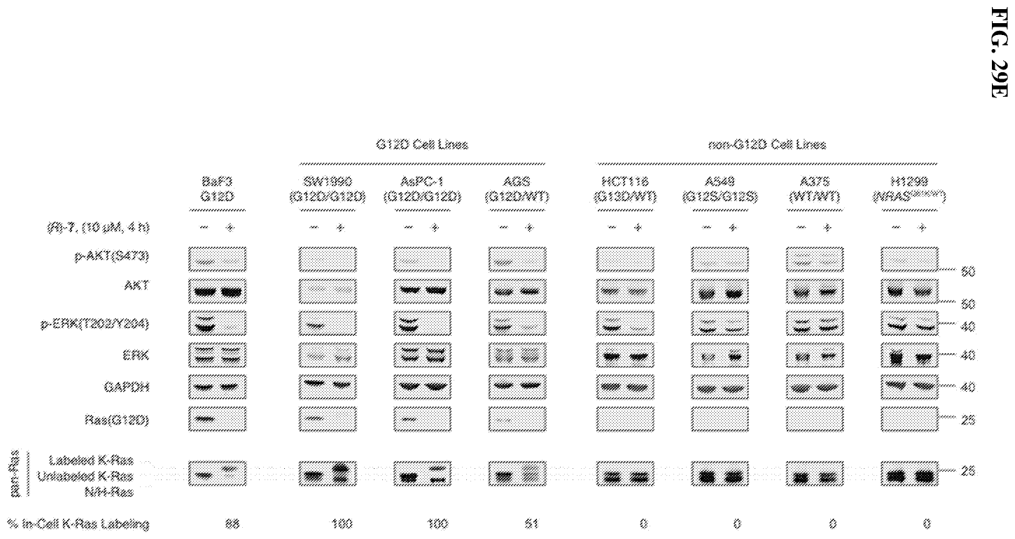

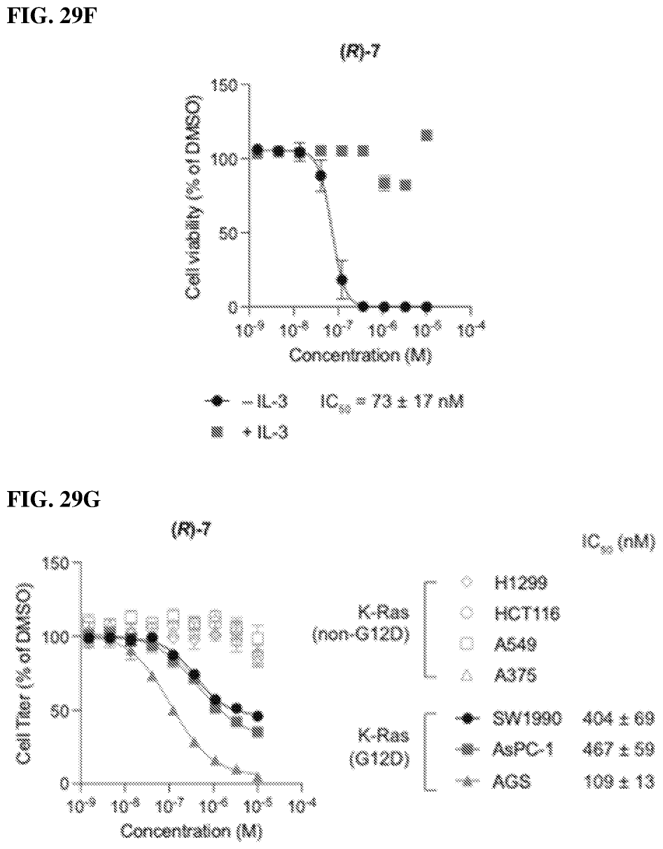

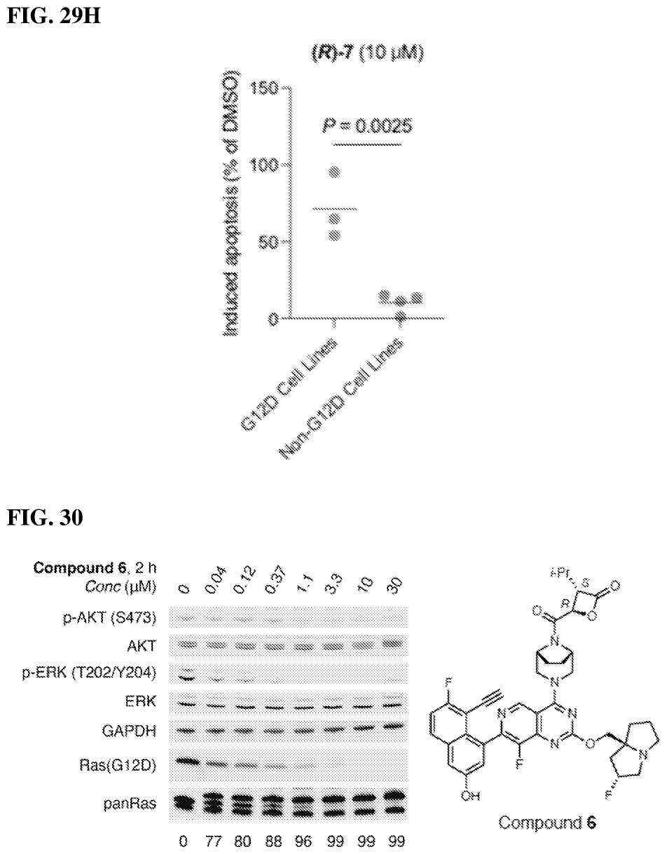

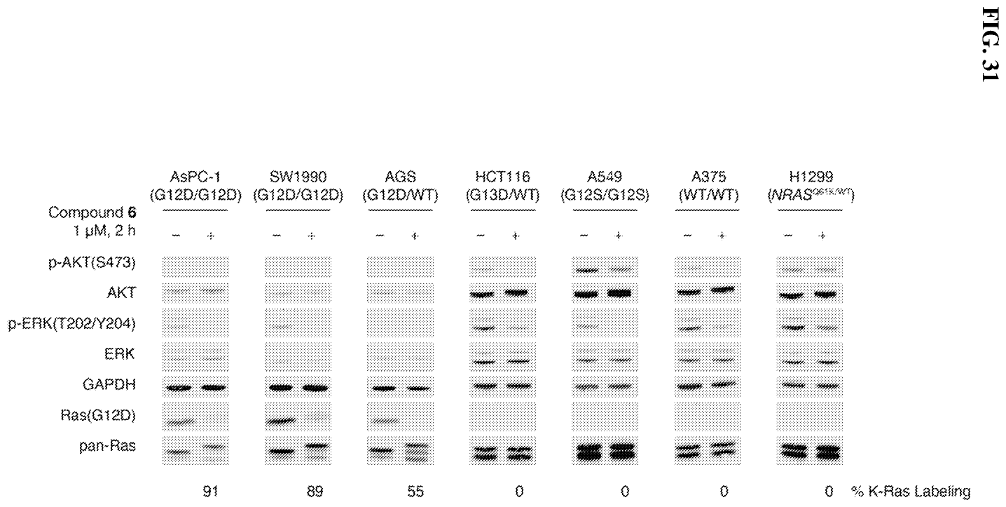

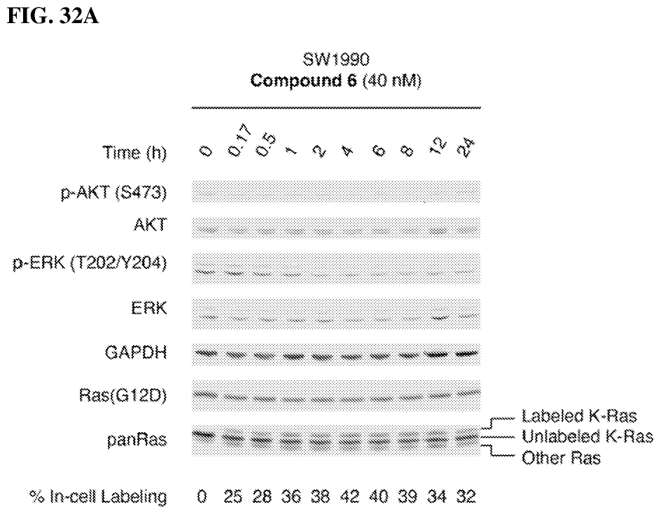

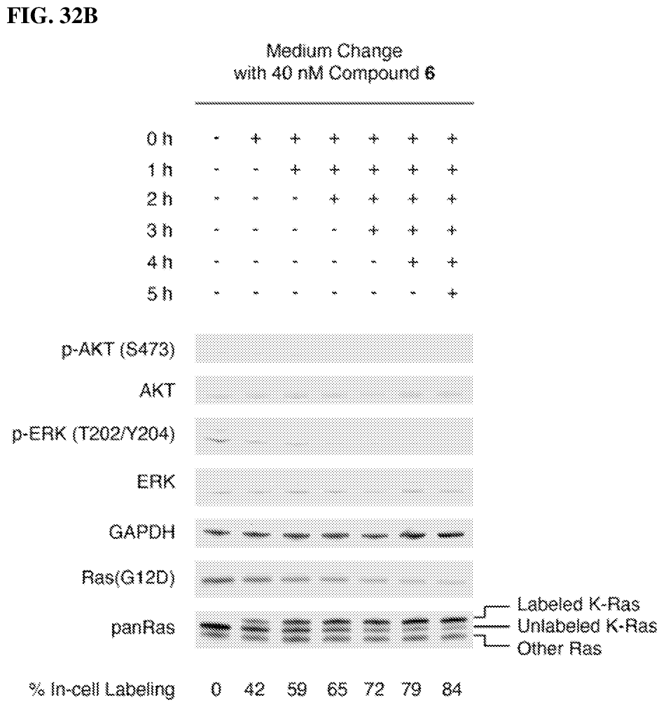

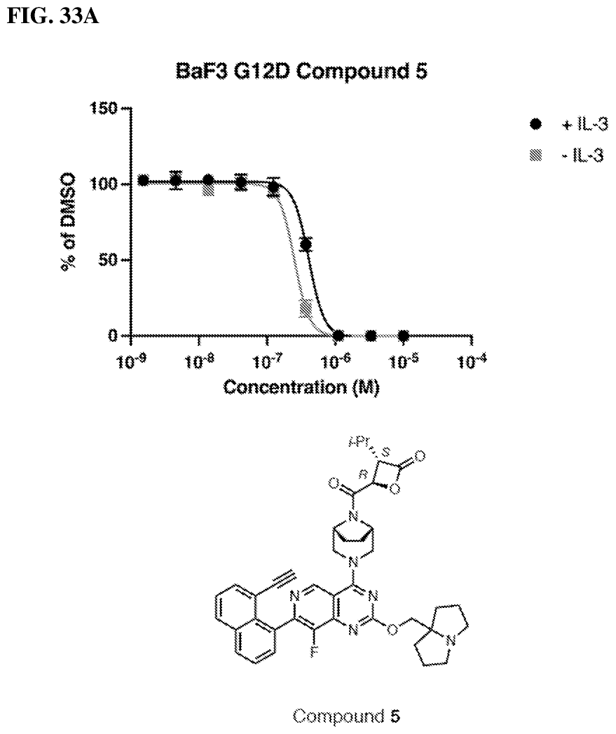

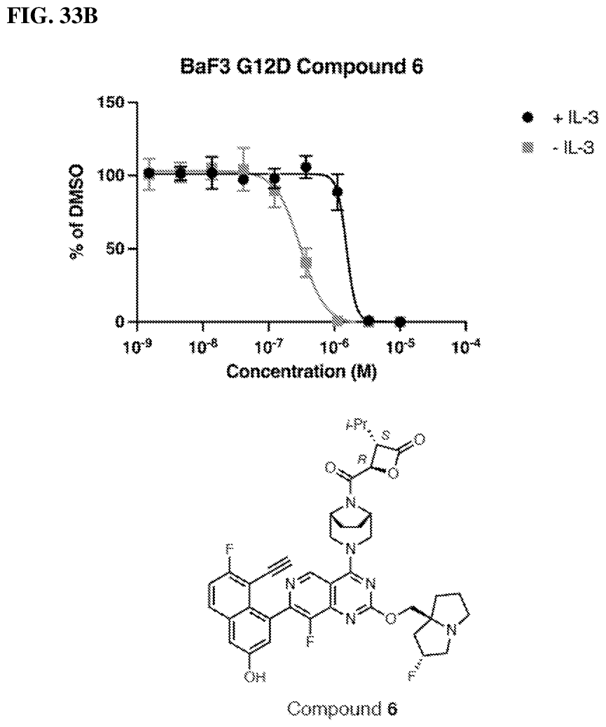

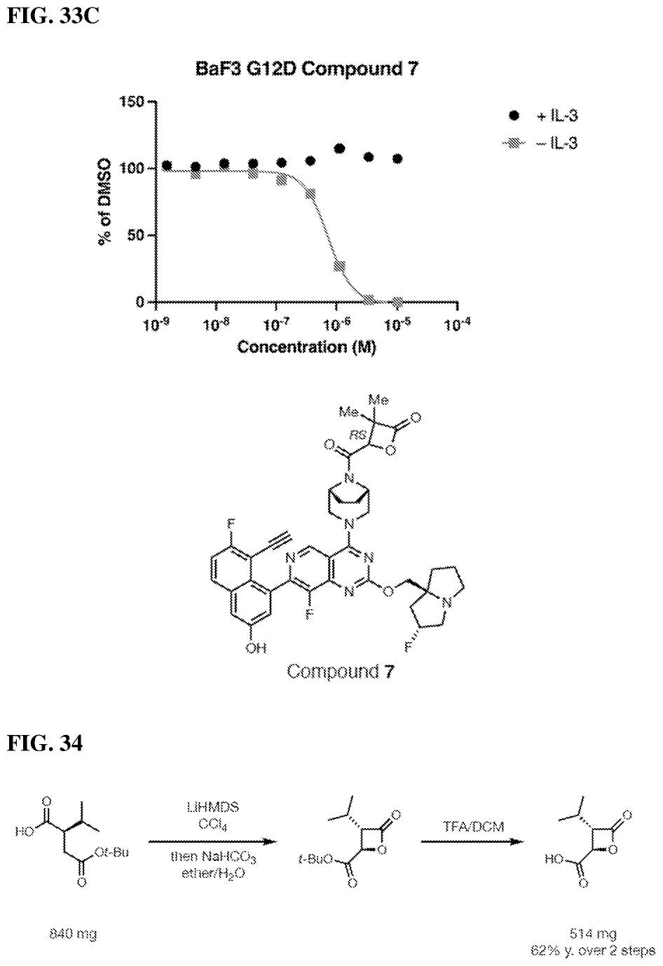

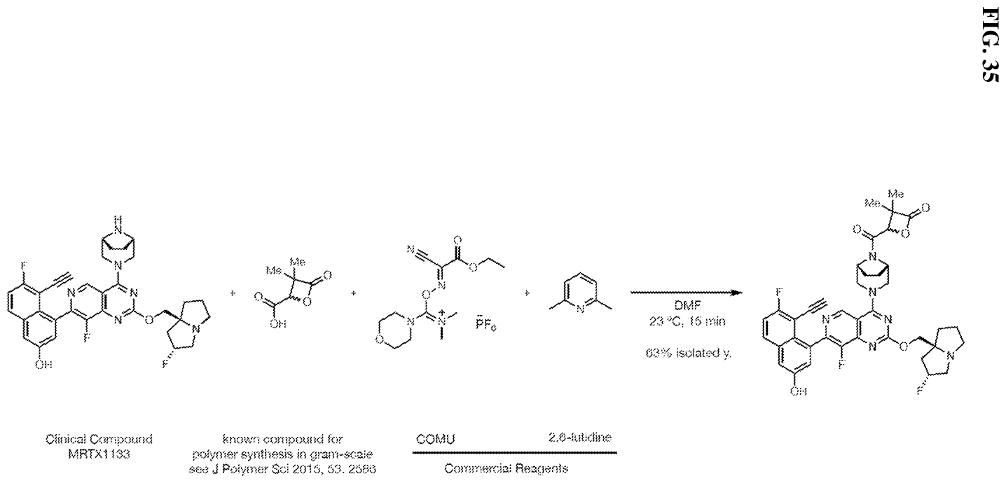

A second substitution was introduced to the pro-R position leading to a 3,3-gem-dimethyl substituted Compound (R)-7. Compound (R)-7 showed comparable labeling kinetics to compound 5; it completely labeled K-Ras(G12D)·GDP and K-Ras(G12D)·GppNHp within 30 min and 300 min, respectively (FIG. 29B). The potency of this molecule against K-Ras(G12D)·GDP was assessed by k_{inact}/K_{I}, 11 mM⁻¹ s⁻¹. This molecule showed high mutant selectivity, labeling only GDP and GppNHp state of G12D but not that of WT, G12E, G12S, or G13D (FIG. 29D).

The mutant-selective, in-cell covalent labeling was further demonstrated by Western blot, where (R)-7 labeled endogenous K-Ras(G12D) completely in homozygous KRAS^{G12D/G12D} cell lines SW1990 and AsPC-1, only half of the two alleles in heterozygous KRAS^{G12D/WT} AGS cell line, and none in non-G12D mutation cell lines (FIG. 29E). Compound (R)-7 inhibited growth of Ba/F3 with an IC₅₀ 73±17 nM. The same cells co-treated with interleukin-3 (IL-3, 10 ng/mL) lost sensitivity to (R)-7 up to 10 μM suggesting the observed cell killing was due to an on-target inhibition. This compound furthered showed significantly differentiated inhibition profile (10 μM, P=0.0025) between KRAS G12D mutation (SW1990, AsPC-1 and AGS) and non-G12D mutation cancer cell lines (H1299, HCT116, A549, A375). Compound (R)-7 showed minimal toxicity to the latter cell lines up to 10 μM concentration.

### REFERENCES FOR EXAMPLE 4

1. I. A. Prior, F. E. Hood, J. L. Hartley, Cancer Res. 2020; 80: 2969. 2. M. E. Welsch, A. Kaplan, J. M. Chambers, M. E. Stokes, P. H. Bos, A. Zask, Y. Zhang, M. Sanchez-Martin, M. A. Badgley, C. S. Huang, T. H. Tran, H. Akkiraju, L. M. Brown, R. Nandakumar, S. Cremers, W. S. Yang, L. Tong, K. P. Olive, A. Ferrando, B. R. Stockwell, Cell 2017; 168: 878. 3. D. Kessler, M. Gmachl, A. Mantoulidis, L. J. Martin, A. Zoephel, M. Mayer, A. Gollner, D. Covini, S. Fischer, T. Gerstberger, T. Gmaschitz, C. Goodwin, P. Greb, D. Haring, W. Hela, J. Hoffmann, J. Karolyi-Oezguer, P. Knesl, S. Kornigg, M. Koegl, R. Kousek, L. Lamarre, F. Moser, S. Munico-Martinez, C. Peinsipp, J. Phan, J. Rinnenthal, J. Sai, C. Salamon, Y. Scherbantin, K. Schipany, R. Schnitzer, A. Schrenk, B. Sharps, G. Siszler, Q. Sun, A. Waterson, B. Wolkerstorfer, M. Zeeb, M. Pearson, S. W. Fesik, D. B. McConnell, Proc. Natl. Acad. Sci. U.S.A. 2019; 116: 15823. 4. H. Feng, Y. Zhang, P. H. Bos, J. M. Chambers, M. M. Dupont, B. R. Stockwell, Biochemistry 2019; 58: 2542. 5. Z. Zhang, R. Gao, Q. Hu, H. Peacock, D. M. Peacock, S. Dai, K. M. Shokat, H. Suga, 2020; DOI 10.1021/acscentsci.0c00514. 6. X. Wang, S. Allen, J. F. Blake, V. Bowcut, D. M. Briere, A. Calinisan, J. R. Dahlke, J. B. Fell, J. P. Fischer, R. J. Gunn, J. Hallin, J. Laguer, J. D. Lawson, J. Medwid, B. Newhouse, P. Nguyen, J. M. O'Leary, P. Olson, S. Pajk, L. Rahbaek, M. Rodriguez, C. R. Smith, T. P. Tang, N. C. Thomas, D. Vanderpool, G. P. Vigers, J. G. Christensen, M. A. Marx, J. Med. Chem. 2021; DOI 10.1021/acs.jmedchem.1c01688. 7. Z. Mao, H. Xiao, P. Shen, Y. Yang, J. Xue, Y. Yang, Y. Shang, L. Zhang, X. Li, Y. Zhang, Y. Du, C.-C. Chen, R.-T. Guo, Y. Zhang, bioRxiv 2021; 2021.12.13.472365. 8. J. M. Ostrem, U. Peters, M. L. Sos, J. A. Wells, K. M. Shokat, Nature 2013; 503: 548. 9. M. R. Janes, J. Zhang, L.-S. Li, R. Hansen, U. Peters, X. Guo, Y. Chen, A. Babbar, S. J. Firdaus, L. Darjania, J. Feng, J. H. Chen, S. Li, S. Li, Y. O. Long, C. Thach, Y. Liu, A. Zarieh, T. Ely, J. M. Kucharski, L. V. Kessler, T. Wu, K. Yu, Y. Wang, Y. Yao, X. Deng, P. P. Zarrinkar, D. Brehmer, D. Dhanak, M. V. Lorenzi, D. Hu-Lowe, M. P. Patricelli, P. Ren, Y. Liu, Cell 2018; 172: 578. 10. B. A. Lanman, J. R. Allen, J. G. Allen, A. K. Amegadzie, K. S. Ashton, S. K. Booker, J. J. Chen, N. Chen, M. J. Frohn, G. Goodman, D. J. Kopecky, L. Liu, P. Lopez, J. D. Low, V. Ma, A. E. Minatti, T. T. Nguyen, N. Nishimura, A. J. Pickrell, A. B. Reed, Y. Shin, A. C. Siegmund, N. A. Tamayo, C. M. Tegley, M. C. Walton, H. L. Wang, R. P. Wurz, M. Xue, K. C. Yang, P. Achanta, M. D. Bartberger, J. Canon, L. S. Hollis, J. D. McCarter, C. Mohr, K. Rex, A. Y. Saiki, T. San Miguel, L. P. Volak, K. H. Wang, D. A. Whittington, S. G. Zech, J. R. Lipford, V. J. Cee, J. Med. Chem. 2020; 63: 52. 11. J. B. Fell, J. P. Fischer, B. R. Baer, J. F. Blake, K. Bouhana, D. M. Briere, K. D. Brown, L. E. Burgess, A. C. Burns, M. R. Burkard, H. Chiang, M. J. Chicarelli, A. W. Cook, J. J. Gaudino, J. Hallin, L. Hanson, D. P. Hartley, E. J. Hicken, G. P. Hingorani, R. J. Hinklin, M. J. Mejia, P. Olson, J. N. Otten, S. P. Rhodes, M. E. Rodriguez, P. Savechenkov, D. J. Smith, N. Sudhakar, F. X. Sullivan, T. P. Tang, G. P. Vigers, L. Wollenberg, J. G. Christensen, M. A. Marx, J. Med. Chem. 2020; 63: 6679. 12. L. M. McGregor, M. L. Jenkins, C. Kerwin, J. E. Burke, K. M. Shokat, Biochemistry 2017; 56: 3178. 13. H.-L. Wang, V. J. Cee, 2021, WO/2021/081212. 14. A. Q. Hassan, C. A. Kirby, W. Zhou, T. Schuhmann, R. Kityk, D. R. Kipp, J. Baird, J. Chen, Y. Chen, F. Chung, D. Hoepfner, N. R. Movva, R. Pagliarini, F. Petersen, C. Quinn, D. Quinn, R. Riedl, E. K. Schmitt, A. Schitter, T. Stams, C. Studer, P. D. Fortin, M. P. Mayer, H. Sadlish, Chem. Biol. 2015; 22: 87. 15. G. A. LoGrippo, Ann. N. Y. Acad. Sci. 1960; 83: 578. 16. E. I. Budowsky, E. A. Friedman, N. V. Zheleznova, F. S. Noskov, Vaccine 1991; 9: 398. 17. C. Fan, X. Ye, Z. Ku, L. Kong, Q. Liu, C. Xu, Y. Cong, Z. Huang, J. Virol. 2017; 91; DOI 10.1128/jvi.00038-17. 18. Q. Gao, L. Bao, H. Mao, L. Wang, K. Xu, M. Yang, Y. Li, L. Zhu, N. Wang, Z. Lv, H. Gao, X. Ge, B. Kan, Y. Hu, J. Liu, F. Cai, D. Jiang, Y. Yin, C. Qin, J. Li, X. Gong, X. Lou, W. Shi, D. Wu, H. Zhang, L. Zhu, W. Deng, Y. Li, J. Lu, C. Li, X. Wang, W. Yin, Y. Zhang, C. Qin, Science 2020; 369: 77. 19. H. Wang, Y. Zhang, B. Huang, W. Deng, Y. Quan, W. Wang, W. Xu, Y. Zhao, N. Li, J. Zhang, H. Liang, L. Bao, Y. Xu, L. Ding, W. Zhou, H. Gao, J. Liu, P. Niu, L. Zhao, W. Zhen, H. Fu, S. Yu, Z. Zhang, G. Xu, C. Li, Z. Lou, M. Xu, C. Qin, G. Wu, G. F. Gao, W. Tan, X. Yang, Cell 2020; 182: 713. 20. J. P. Uittenbogaard, B. Zomer, P. Hoogerhout, B. Metz, 2011; 286: 36198. 21. Y. M. She, K. Cheng, A. Farnsworth, X. Li, T. D. Cyr, Proteomics 2013; 13: 3537. 22. Z. Zhang, K. Z. Guiley, K. M. Shokat, Nat. Chem. Biol. 2022; 18: 1177. 23. J. C. Hunter, A. Manandhar, M. A. Carrasco, et al., Mol. Cancer Res. 2015; 13: 1325. 24. C. W. Johnson, Y. Lin, D. Reid, et al., CellReports 2019; 28: 1538.

### Example 5: Additional Experimental Methods

### Cell Culture

Ba/F3 cells were a gift from Dr. Trevor Bivona (UCSF) and were maintained in RPMI 1640 (Gibco 11875093) supplemented with 10% heat-inactivated fetal bovine serum (Axenia Biologix) and 10 ng/mL recombinant mouse interleukin-3 (Gibco PMC0031). AsPc-1 cells were obtained from ATCC (CRL-1682) and maintained in RPMI 1640 (Gibco 11875093) supplemented with 10% heat-inactivated fetal bovine serum (Axenia Biologix). SW1990 cells were obtained from ATCC (CRL-2172) and maintained in high-glucose (4.5 g/L) DMEM (Gibco 11995073) supplemented with 4 mM L-glutamine, 1 mM sodium pyruvate, and 10% heat-inactivated fetal bovine serum (Axenia Biologix).

Cells were passed for at least two generations after cryorecovery before they were used for assays. All cell lines were tested mycoplasma negative using MycoAlert™ Mycoplasma Detection Kit (Lonza). When indicated, cells were treated with drugs at 40-60% confluency at a final DMSO concentration of 1%. At the end of treatment period, cells were placed on ice. Unless otherwise indicated, adherent cells were washed once with ice-cold PBS (1 mL), scraped with a spatula, and pelleted by centrifugation (500×g, 5 min). Suspension cells were pelleted by centrifugation (500×g, 5 min), washed with 1 mL ice-cold PBS, and pelleted again. Cells were lysed in RIPA buffer supplemented with protease and phosphatase inhibitors (cOmplete and phosSTOP, Roche) on ice for 10 min. For RBD pulldown experiments, cells were lysed in Co-IP Lysis Buffer in lieu of RIPA buffer. Lysates were clarified by high-speed centrifugation (19,000×g, 10 min). Concentrations of lysates were determined with protein BCA assay (Thermo Fisher) and adjusted to 2 mg/mL with additional RIPA buffer (or Co-IP Lysis Buffer). Samples were mixed with 5×SDS Loading Dye and heated at 95° C. for 5 min.

### Gel Electrophoresis and Immunoblot

Unless otherwise noted, SDS-PAGE was run with Novex 12% Bis-Tris gel (Invitrogen) in MES running buffer (Invitrogen) at 200 V for 60 min following the manufacturer's instructions. Protein bands were transferred onto 0.2-μm nitrocellulose membranes (Bio-Rad) using a wet-tank transfer apparatus (Bio-Rad Criterion Blotter) in lx TOWBIN buffer with 10% methanol at 75V for 45 min. Membranes were blocked in 5% BSA-TBST for 1 h at 23° C. Primary antibody binding was performed with the indicated antibodies diluted in 5% BSA-TBST at 4° C. for at least 16 h. After washing the membrane three times with TBST (5 min each wash), secondary antibodies (goat anti-rabbit IgG-IRDye 800 and goat anti-mouse IgG-IRDye 680, Li-COR) were added as solutions in 5% skim milk-TBST at the dilutions recommended by the manufacturer. Secondary antibody binding was allowed to proceed for 1 h at 23° C. The membrane was washed three times with TBST (5 min each wash) and imaged on a Li-COR Odyssey fluorescence imager.

### Preparation of Mouse Stem Cell Virus (MSCV)

pMSCV-Puro plasmids containing full length human KRAS genes (wildtype, G12D) were constructed using standard molecule biology techniques by inserting the KRAS gene fragment between the BamHI and XhoI sites. Transfection-grade plasmids were prepared using ZymoPure II Plasmid Midiprep kit. EcoPack 293 cells (Takara Bio) were plated in 6-well plates (3×10⁵/mL, 2 mL). The next day, cells were transfected with 2.5 μg pMSCV plasmid using lipofectamine 3000 following the manufacturer's instructions. The cells were incubated for 66 h, and then the virus-containing supernatants were collected and passed through a 0.22-μm syringe filter. The harvested virus was used immediately for spinfection of Ba/F3 cells or stored at −80° C.

### Generation of Stable Ba/F3 Transductants

1 mL of MSCV-containing supernatant (vide supra) was added to one well of a 6-well plate containing 1×10⁶ Ba/F3 cells in 1 mL of media comprised of 60% RMPI 1640, 40% heat-inactivated FBS, 10 ng mouse IL-3 and 4 μg polybrene. Cells were spinfected by centrifugation at 2,000 g for 90 minutes at room temperature and then placed in the incubator for 24 hours. After 1 day, the cells were diluted into 10 mL culture medium (RPMI 1640+10% heat-inactivated FBS, 10 ng/mL mouse IL-3) and recovered for a second day after spinfection. On the third day after spinfection, cells were pelleted at 500×g for 5 min and resuspended in 10 mL selection medium (RPMI 1640+10% heat-inactivated FBS, 10 ng/mL mouse IL-3, 1.25 μg/mL puromycin). Cells were maintained under puromycin selection for 4-7 days, splitting as required to maintain density <2×10⁶ cells/mL. After 7 days, cells were pelleted, washed once with IL-3 free culture medium (RPMI 1640+10% heat-inactivated FBS) and pelleted again before resuspending at 2-4×10⁵ cells/mL in IL-3 free culture medium. Cells were maintained under these conditions for 7 days, passaging as needed to maintain density <2×10⁶ cells/mL. Growth was monitored (Countess II Cell Counter) over these 7 days to confirm that an IL-3 independent population has been achieved.

### Differential Scanning Fluorimetry

The protein of interest was diluted with SEC Buffer [20 mM HEPES 7.5, 150 mM NaCl, 1 mM MgCl₂] to 8 μM. This solution was dispensed into wells of a white 96-well PCR plate in triplicate (25 μL/well). Fluorescence was measured at 0.5-° C. temperature intervals every 30 s from 25° C. to 95° C. on a Bio-Rad CFX96 qPCR system using the FRET setting. Each data set was normalized to the highest fluorescence and the normalized fluorescence reading was plotted against temperature in GraphPad Prism 8.0. Tm values were determined as the temperature(s) corresponding to the maximum(ma) of the first derivative of the curve.

### Detection of Covalent Modification of K-Ras by Whole-Protein Mass Spectrometry

Test compounds were prepared as 100× stock solutions in DMSO. K-Ras proteins were diluted with SEC Buffer (20 mM HEPES 7.5, 150 mM NaCl, 1 mM MgCl₂) to 400 nM or 1 μM. In a typical reaction, 0.5 μL 100× compound stock was mixed with 50 μL diluted K-Ras protein, and the resulting mixture was incubated for the desired amount of time. The extent of modification was assessed by electrospray mass spectrometry using a Waters Xevo G2-XS system equipped with an Acquity UPLC BEH C4 1.7 μm column. The mobile phase was a linear gradient of 5-95% acetonitrile/water+0.05% formic acid. For kinetic measurements, a 2× compound solution was first prepared in SEC Buffer, which was then mixed with 400 nM K-Ras(G12D) protein at 1:1 (v/v) ratio. Injection time stamps were used to calculate elapsed time.

### Detection of Covalent Modification of K-Ras by Tandem Mass Spectrometry

K-Ras(G12D) protein (1 μM, 100 μL) in PBS 7.4 was treated with β-propiolactone (1 mM or 10 mM) at 23° C. for 1 h. The reaction buffer was exchanged into Digestion Buffer (20 mM Tris 8.0, 2 mM CaCl₂)) using a Zeba 0.5-mL desalting column (7K MWCO, Thermo Scientific). 80 μL of the resulting protein solution was mixed with 2 μL 200 mM DTT. The mixture was heated at 56° C. for 30 min. After cooling to 23° C., 4 μL 200 mM iodoacetamide was added. After 15 min at 23° C., 2.1 μL 200 mM DTT was added. After an additional 5 min, 500 ng trypsin was added to the mixture, and the samples were incubated at 37° C. overnight. 5 μL 10% formic acid was added to stop the digestion (final formic acid concentration: 0.5% v/v). The tryptic peptides were enriched and desalted using OMIX C18 tips (Agilent) following the manufacturer's instructions. Peptides (0.5% of total) were resolved on an Easy-Spray nano-HPLC column (Thermo Fisher ES800A, 150 mm length, 3 μL particle size, 100-A particle size) over a 54-min gradient of 2-37% acetonitrile-water+0.1% formic acid and analyzed by a Q-Exactive hybrid quadrupole-orbitrap mass spectrometer (MS1 resolution 70,000, AGC target: 3e6, range: 350-1500 m/z; MS2 resolution 17,500, AGC target: 5e4, max injection time: 120 ms, Top 10, NCE: 25%, dynamic exclusion: 15 s). Peptides were searched against the K-Ras(G12D) sequence using MaxQuant (v.2.0.3.0, https://www.maxquant.org/), with β-propiolactone (C₃H₄O₂) as a variable modification on serine, threonine, lysine, aspartate, tyrosine, glutamate and histidine residues. Peptides were identified with a false discovery rate cutoff of 0.01. Only the peptides with sufficient MS2 fragment information to assign the modification site with >0.9 probability were used for analysis.

### Cell Viability Assay

Cells were seeded into 96-well white flat bottom plates (1,000 cells/well) (Corning) and incubated overnight. Cells were treated with the indicated compounds in a nine-point threefold dilution series (100 μL final volume) and incubated for 72 h. Cell viability was assessed using a commercial CellTiter-Glo (CTG) luminescence-based assay (Promega). Briefly, the 96-well plates were equilibrated to room temperature before the addition of diluted CTG reagent (100 L) (1:4 CTG reagent:PBS). Plates were placed on an orbital shaker for 30 min before recording luminescence using a Spark 20M (Tecan) plate reader.

### Recombinant Protein Expression and Purification

K-Ras(wildtype), K-Ras(G12D), K-Ras(G12E), K-Ras(G13D), K-Ras (G12D) Cyslight

DNA sequences encoding human K-Ras (wildtype, aa 1-169), human K-Ras (G12D, aa 1-169), human K-Ras (G12E, aa 1-169), human K-Ras (G13D, aa 1-169), and human K-Ras G12D Cyslight (G12D/C51S/C80L/C118S, aa 1-169) were codon optimized, synthesized by Twist Biosciences and cloned into pJExpress411 vector using the Gibson Assembly method (7). The resulting construct contains N-terminal 6×His tag and a TEV cleavage site (ENLYFQG). The proteins were expressed and purified following previously reported protocols (8,9). Briefly, chemically competent BL21(DE3) cells were transformed with the corresponding plasmid and grown on LB agar plates containing 50 μg/mL kanamycin. A single colony was used to inoculate a culture at 37° C., 220 rpm in terrific broth containing 50 μg/mL kanamycin. When the optical density reached 0.6, the culture temperature was reduced to 20° C., and protein expression was induced by the addition of IPTG to 1 mM. After 16 h at 20° C., the cells were pelleted by centrifugation (6,500×g, 10 min) and lysed in lysis buffer [20 mM Tris 8.0, 500 mM NaCl, 5 mM imidazole] with a high-pressure homogenizer (Microfluidics, Westwood, MA). The lysate was clarified by high-speed centrifugation (19,000×g, 15 min) and the supernatant was used in subsequent purification by immobilized metal affinity chromatography (IMAC). His-TEV tagged protein was captured with Co-TALON resin (Clonetech, Takara Bio USA, 2 mL slurry/liter culture) at 4° C. for 1 h with constant end-to-end mixing. The loaded beads were then washed with lysis buffer (50 mL/liter culture) and the protein was eluted with elution buffer [20 mM Tris 8.0, 300 mM NaCl, 300 mM imidazole]. To this protein solution was added His-tagged TEV protease (0.05 mg TEV/mg Ras protein) and GDP (1 mg/mg Ras protein), and the mixture was dialyzed against TEV Cleavage Buffer [20 mM Tris 8.0, 300 mM NaCl, 1 mM EDTA, 1 mM DTT] at 4° C. using a 10K MWCO dialysis cassette until LC-MS analysis showed full cleavage (typically 16-24 h). MgCl₂ was added to a final concentration of 5 mM, and the mixture was incubated with 1 mL Ni-NTA (Qiagen) beads at 4° C. for 1 h to remove TEV protease, any residual His-tagged proteins and peptides. The protein solution was diluted 1:10 v/v with 20 mM Tris 8.0 and further purified with anion exchange chromatography (HiTrapQ column, GE Healthcare Life Sciences) using a NaCl gradient of 50 mM to 500 mM in 20 mM Tris 8.0. Nucleotide loading was performed by mixing the ion exchange-purified protein with an excess of GDP (5 mg/liter culture) or GppNHp (5 mg/liter culture) and 5 mM EDTA at 23° C. for 30 min. The reaction was stopped by the addition of MgCl₂ to 10 mM. For GppNHp, an additional calf intestine phosphatase treatment was performed as follows to ensure high homogeneity of the loaded nucleotide. The protein buffer was exchanged into Phosphatase Buffer [32 mM Tris 8.0, 200 mM ammonium sulfate, 0.1 mM ZnCl₂] with a HiTrap Desalting Column (GE Healthcare Life Sciences). To the buffer-exchanged protein solutions, GppNHp was added to 5 mg/mL, and Calf Intestine Phosphatase (NEB) was added to 10 U/mL. The reaction mixture was incubated on ice for 1 h, and MgCl₂ was added to a final concentration of 20 mM. After nucleotide loading, the protein was concentrated using an 10K MWCO centrifugal concentrator (Amicon-15, Millipore) to 20 mg/mL and purified by size exclusion chromatography on a Superdex 75 10/300 GL column (GE Healthcare Life Sciences). Fractions containing pure Ras protein were pooled and concentrated to 20 mg/mL and stored at −78° C. In our hands, this protocol gives a typical yield of 5-15 mg/liter culture.

### Crystallization

K-Ras(G12D) Cyslight (G12D/C51S/C80L/C118S) bound by GDP purified by size exclusion chromatography was diluted to 100 μM in Reaction Buffer (20 mM HEPES 7.5, 150 mM NaCl, 1 mM MgCl₂). For co-crystal structures, compound 1 was added as a 10 mM solution in DMSO to a final concentration of 200 μM. The mixture was allowed to stand at 23° C. until LC-MS analysis of the reaction mixture showed full conversion to a single covalent adduct. The reaction mixture was purified by size exclusion chromatography (Superdex75, 20 mM HEPES 7.5, 150 mM NaCl, 1 mM MgCl₂) and concentrated to 20 mg/mL. For crystallization, 0.1 μL of the protein was mixed with 0.1 μL well buffer. Crystals were grown at 20° C. in a 96-well plate using the hanging-drop vapor diffusion method. Maximal crystal growth was achieved after 7 days. The crystals were transferred to a cryoprotectant solution (same composition as the well solution with 20% glycerol) and flash-frozen in liquid nitrogen.

### X-Ray Data Collection and Structure Determination

Dataset was collected at the Advanced Light Source beamline 8.2.2 with X-ray at a wavelength of 0.999907 Å. The dataset was indexed and integrated using iMosflm (Battye et al., 2011), scaled with Scala (Evans, 2006) and solved by molecular replacement using Phaser (McCoy et al., 2007) in CCP4 software suite (Winn et al., 2011). The crystal structure of GDP-bound K-Ras(G12C)-MRTX849 adduct (PDB code: 6USZ) was used as the initial model. The structure was manually refined with Coot (Emsley et al., 2010) and PHENIX (Adams et al., 2010).

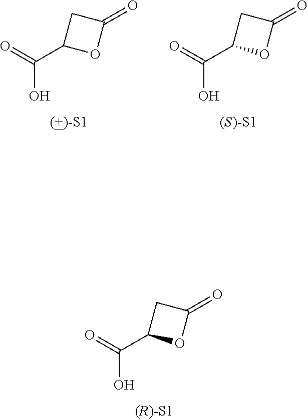 [SMILES: O=C1CC(C(=O)O)O1 ; O=C1C[C@@H](C(=O)O)O1 ; O=C1C[C@H](C(=O)O)O1]

(±)—S1, (S)—S1, and (R)—S1 were prepared following reported protocols (1) from DL-malic acid, L-malic acid, and D-malic acid, respectively.

(2R,3S)—S4, (2R,3S)—S7, and (2S,3R)—S7 were prepared using an protocol adapted from Tello-Aburto et al. (2).

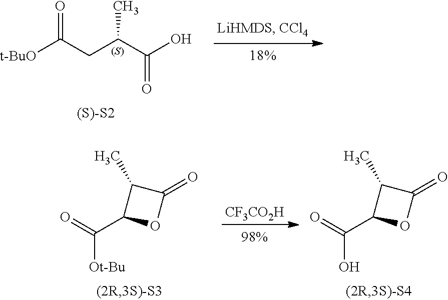

### tert-Butyl (2R,3S)-3-methyl-4-oxo-oxetane-2-carboxylate [(2R,3S)—S3]

A solution of (S)-4-tert-butoxy-2-methyl-4-oxo-butanoic acid (200 mg, 1.06 mmol) in THF (1.06 mL) was added dropwise to a solution of Lithium bis(trimethylsilyl)amide (2.66 mL, 2.656 mmol) in THF at −78° C. The resulting mixture was stirred at −78° C. for 1 h. Freshly distilled carbon tetrachloride (123 μL, 1.27 mmol) was added dropwise to the solution, and the resulting mixture was allowed to warm to 23° C. over 30 min. The mixture was further stirred for 1 h at 23° C. At this point, TLC analysis (50% ethyl acetate-hexanes, after miniworkup in ether/ammonium chloride) showed full consumption of the starting acid. The solvent was removed by rotary evaporation, and the residue was resuspended in ether (10 mL). Saturated aqueous sodium bicarbonate solution (10 mL) was added to the mixture, and the resulting biphasic mixture was stirred vigorously at 23° C. for 12 h. The layers were separated, and the aqueous layer was extracted with ether (2×10 mL). The combined ether layers were washed with saturated aqueous sodium chloride solution. The washed organic phase was dried over magnesium sulfate. The dried solution was filtered, and the filtrate was concentrated under reduced pressure. The residue was purified by column chromatography (0-20% ethyl acetate-hexanes) to afford the product (2R,3S)—S6 as a crystalline solid (35 mg, 18%). ¹H NMR (400 MHz, CDCl₃) δ 4.44 (d, J=4.3 Hz, 1H), 3.74 (qd, J=7.6, 4.3 Hz, 1H), 1.53 (s, 9H), 1.51 (d, J=7.6 Hz, 3H). ¹³C NMR (100 MHz, CDCl₃) δ 169.91, 167.12, 83.71, 73.26, 51.79, 27.93, 27.69, 12.61.

### (2R,3S)-3-Methyl-4-oxo-oxetane-2-carboxylic acid [(2R,3S)—S4]

(2R,3S)—S6 (35 mg, 0.188 mmol) was dissolved in 1:1 dichloromethane:trifluoroacetic acid (1.0 mL) at 23° C. and the resulting solution was allowed to stand at 23° C. for 1 h. The reaction mixture was concentrated under reduced pressure to afford the product as a yellow oil (24 mg, 98%). ¹H NMR (400 MHz, CDCl₃) δ 4.64 (d, J=4.3 Hz, 1H), 3.91 (qd, J=7.6, 4.3 Hz, 1H), 1.59 (d, J=7.6 Hz, 3H).

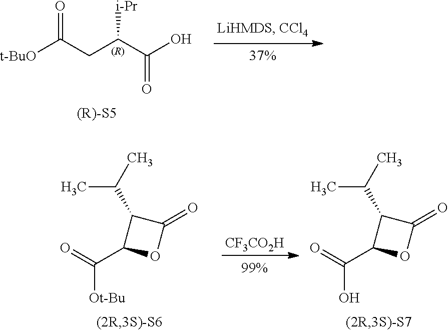

### tert-Butyl (2R,3S)-3-isopropyl-4-oxo-oxetane-2-carboxylate [(2R,3S)—S6]

A solution of (2R)-4-tert-butoxy-2-isopropyl-4-oxo-butanoic acid (250 mg, 1.16 mmol) in THF (1.06 mL) was added dropwise to a solution of Lithium bis(trimethylsilyl)amide (2.89 mL, 2.89 mmol) in THF at −78° C. The resulting mixture was stirred at −78° C. for 1 h. Freshly distilled carbon tetrachloride (134 μL, 1.39 mmol) was added dropwise to the solution, and the resulting mixture was allowed to warm to 23° C. over 30 min. The mixture was further stirred for 1 h at 23° C. At this point, TLC analysis (50% ethyl acetate-hexanes, after mini-workup in ether/ammonium chloride) showed full consumption of the starting acid. The solvent was removed by rotary evaporation, and the residue was resuspended in ether (10 mL). Saturated aqueous sodium bicarbonate solution (10 mL) was added to the mixture, and the resulting biphasic mixture was stirred vigorously at 23° C. for 12 h. The layers were separated, and the aqueous layer was extracted with ether (2×10 mL). The combined ether layers were washed with saturated aqueous sodium chloride solution. The washed organic phase was dried over magnesium sulfate. The dried solution was filtered, and the filtrate was concentrated under reduced pressure. The residue was purified by column chromatography (0-100% ethyl acetate-hexanes) to afford the product (2R,3S)—S6 as a pale-yellow oil (86 mg, 37%). ¹H NMR (400 MHz, CDCl₃) δ 4.54 (d, J=4.4 Hz, 1H), 3.47 (dd, J=8.5, 4.4 Hz, 1H), 2.20 (dhept, J=8.5, 6.7 Hz, 1H), 1.53 (s, 9H), 1.14 (d, J=6.7 Hz, 3H), 1.09 (d, J=6.7 Hz, 3H). ¹³C NMR (100 MHz, CDCl₃) δ 168.74, 167.42, 83.52, 70.16, 64.00, 27.92, 27.70, 19.98, 19.63.

### (2R,3S)-3-isopropyl-4-oxo-oxetane-2-carboxylic acid [(2R,3S)—S7]

(2R,3S)—S6 (99 mg, 0.46 mmol) was dissolved in 1:1 trifluoroacetic acid:dichloromethane (1.0 mL) at 23° C. and the mixture was allowed to stand at 23° C. for 1 h. The reaction mixture was concentrated under reduced pressure to afford the product as a pale-yellow oil (73 mg, 99%). ¹H NMR (400 MHz, CDCl₃) δ 4.73 (d, J=4.4 Hz, 1H), 3.66 (dd, J=8.4, 4.4 Hz, 1H), 2.27 (dp, J=8.3, 6.6 Hz, 1H), 1.17 (d, J=6.8 Hz, 3H), 1.13 (d, J=6.7 Hz, 3H).

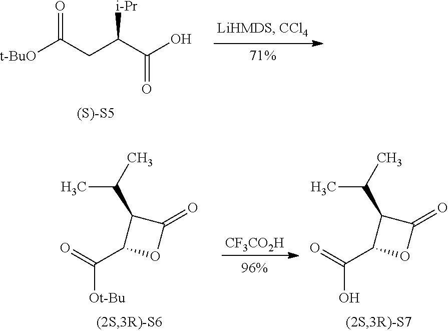

### tert-butyl (2S,3R)-3-isopropyl-4-oxo-oxetane-2-carboxylate [(2S,3R)—S6]

A solution of (S)-4-tert-butoxy-2-isopropyl-4-oxo-butanoic acid (500 mg, 2.31 mmol) in THF (2.31 mL) was added dropwise to a solution of Lithium bis(trimethylsilyl)amide (1.0 M in THF, 5.78 mL, 5.78 mmol) in THF at −78° C. The resulting mixture was stirred at −78° C. for 1 h. Freshly distilled carbon tetrachloride (268 μL, 2.78 mmol) was added dropwise to the solution, and the resulting mixture was allowed to warm to 23° C. over 30 min. The mixture was further stirred for 1 h at 23° C. At this point, TLC analysis (50% ethyl acetate-hexanes, after mini-workup in ether/ammonium chloride) showed full consumption of the starting acid. The solvent was removed by rotary evaporation, and the residue was resuspended in ether (10 mL). Saturated aqueous sodium bicarbonate solution (10 mL) was added to the mixture, and the resulting biphasic mixture was stirred vigorously at 23° C. for 12 h. The layers were separated, and the aqueous layer was extracted with ether (2×10 mL). The combined ether layers were washed with saturated aqueous sodium chloride solution. The washed organic phase was dried over magnesium sulfate. The dried solution was filtered, and the filtrate was concentrated under reduced pressure. The residue was purified by column chromatography (0-100% ethyl acetate-hexanes) to afford the product (2S,3R)—S6 as a pale-yellow oil (350 mg, 71%). Spectral data was identical to that of (2R,3S)—S6.

### (2S,3R)-3-isopropyl-4-oxo-oxetane-2-carboxylic acid [(2S,3R)—S7]

(2S,3R)—S6 (350 mg, 1.64 mmol) was dissolved in 1:1 trifluoroacetic acid:dichloromethane (1.0 mL) at 23° C. and the mixture was allowed to stand at 23° C. for 1 h. The reaction mixture was concentrated under reduced pressure to afford the product as a pale-yellow oil (250 mg, 96%). Spectral data was identical to that of (2R,3S)—S7.

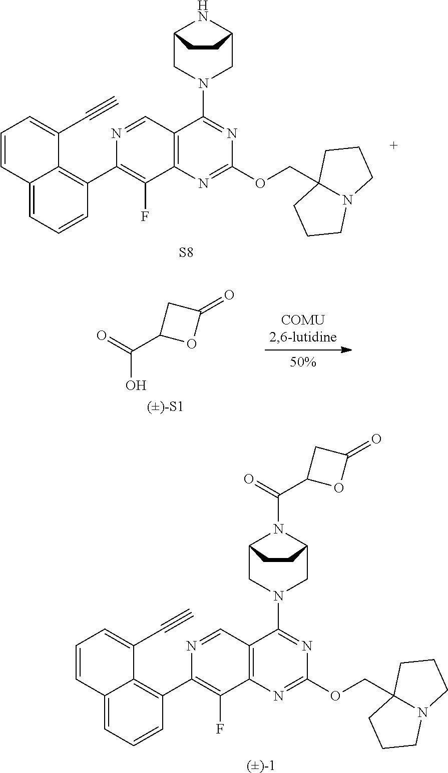

(±)-1, also referred to herein as 14-049.

An oven-dried one-dram vial was charged with (±)—S1 (5.2 mg, 0.045 mmol), COMU (19.4 mg, 0.045 mmol) and a magnetic stir bar. DMF (0.29 mL) and 2,6-lutidine (10 μL, 0.090 mmol) were added sequentially via syringe. The reaction mixture was stirred for 15 min at 23° C., during which the color of the solution turned dark brown. S8³ (10 mg, 0.015 mmol) was added as a solid. In 15 min, LC-MS analysis showed that the starting material had been fully consumed, giving rise to a single product with the expected m/z. The retention time was slightly different from the last time. The reaction mixture was partitioned between saturated aqueous sodium bicarbonate solution (5 mL) and dichloromethane (5 mL). The layers were separated, and the aqueous layer was extracted with dichloromethane (2×5 mL). The combined organic layers were dried over sodium sulfate. The dried solution was filtered, and the filtrate was concentrated. The residue was purified by column chromatography (0-30% methanol-dichloromethane, 4-g RediSep(R) Rf column, Teledyne ISCO, Lincoln, NE) to afford the product as a white power (4.9 mg, 50%). HRMS (C₃₇H₃₅FN₆O₄+H)⁺ Calc'd: 647.2782, Found: 647.2858.

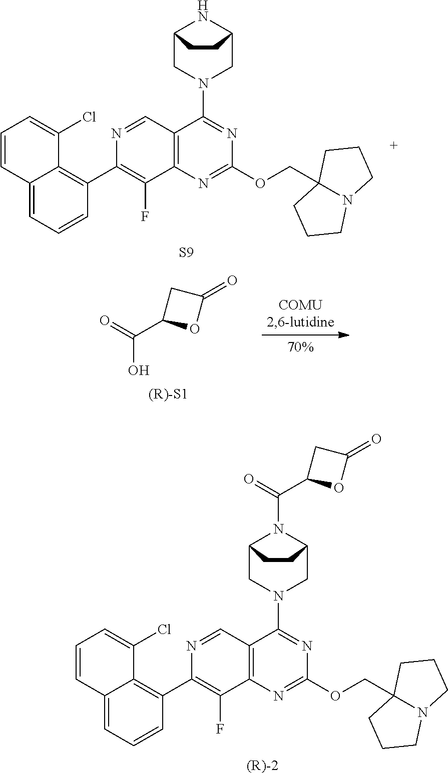

(R)-2, also referred to herein as 14-076.

An oven-dried one-dram vial was charged with (R)—S1 (10 mg, 0.089 mmol), COMU (38 mg, 0.089 mmol) and a magnetic stir bar. DMF (0.292 mL) and 2,6-lutidine (21 μL, 0.18 mmol) were added sequentially via pipette. The reaction mixture was stirred for 15 min at 23° C., during which the color of the solution turned dark brown. 7-(8-chloro-1-naphthyl)-4-(3,8-diazabicyclo[3.2.1]octan-3-yl)-8-fluoro-2-(1,2,3,5,6,7-hexahydropyrrolizin-8-ylmethoxy)pyrido[4,3-d]pyrimidine trifluoroacetate salt³ (20 mg, 0.030 mmol) was added as a solid. In 15 min, LC-MS analysis showed that the starting material had been fully consumed, giving rise to a single product with the expected m/z. The reaction mixture was partitioned between saturated aqueous sodium bicarbonate solution (5 mL) and dichloromethane (5 mL). The layers were separated, and the aqueous layer was extracted with dichloromethane (2×5 mL). The combined organic layers were dried over sodium sulfate. The dried solution was filtered, and the filtrate was concentrated. The residue was purified by column chromatography (0-30% methanol-dichloromethane, 4-g RediSep(R) Rf column, Teledyne ISCO, Lincoln, NE) to afford the product as a white power (13.7 mg, 70%). HRMS (C₃₅H₃₄ClFN₆O₄+H)⁺ Calc'd: 657.2392, Found: 657.2354.

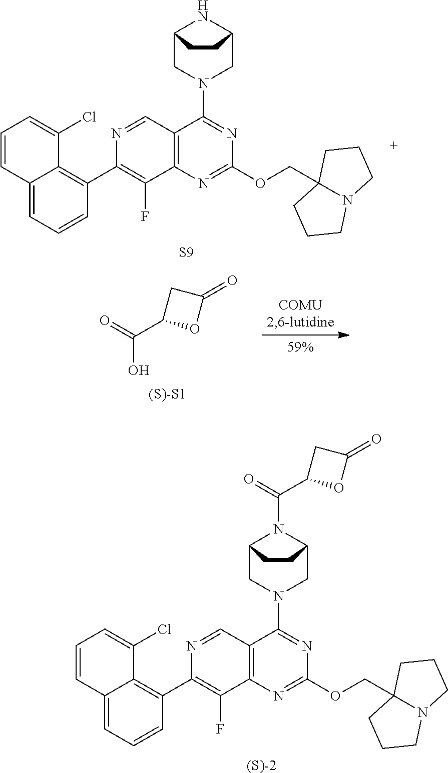

(S)-2, also referred to herein as 14-075.

An oven-dried one-dram vial was charged with (S)—S1 (10 mg, 0.089 mmol), COMU (39 mg, 0.089 mmol) and a magnetic stir bar. DMF (0.292 mL) and 2,6-lutidine (21 μL, 0.181 mmol) were added sequentially via pipette. The reaction mixture was stirred for 15 min at 23° C., during which the color of the solution turned dark brown. S9 (20 mg, 0.030 mmol) was added as a solid. In 15 min, LC-MS analysis showed that the starting material had been fully consumed, giving rise to a single product with the expected m/z. The reaction mixture was partitioned between saturated aqueous sodium bicarbonate solution (5 mL) and dichloromethane (5 mL). The layers were separated, and the aqueous layer was extracted with dichloromethane (2×5 mL). The combined organic layers were dried over sodium sulfate. The dried solution was filtered, and the filtrate was concentrated. The residue was purified by column chromatography (0-30% methanol-dichloromethane, 4-g RediSep(R) Rf column, Teledyne ISCO, Lincoln, NE) to afford the product as a white power (11.5 mg, 59%). HRMS (C₃₅H₃₄ClFN₆O₄+H)⁺ Calc'd: 657.2392, Found: 657.2354.

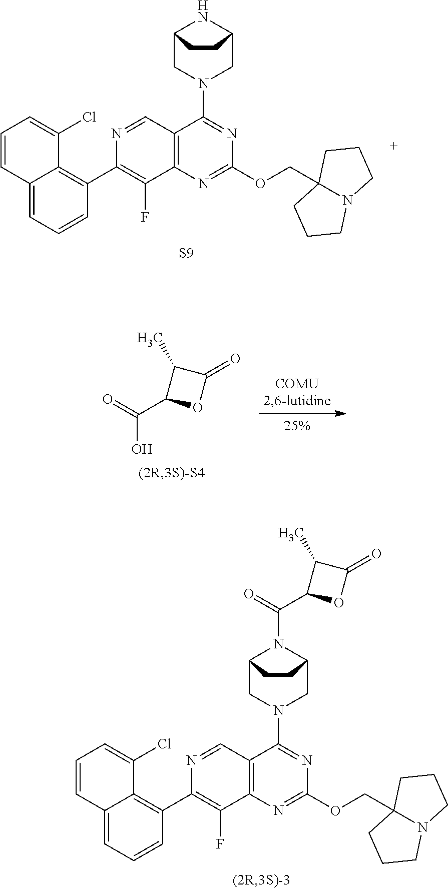

(2R,3S)-3, also referred to herein as 14-082.

An oven-dried one-dram vial was charged with (2R,3S)—S4 (5.8 mg, 0.045 mmol), COMU (19 mg, 0.045 mmol) and a magnetic stir bar. DMF (0.292 mL) and 2,6-lutidine (10 μL, 0.089 mmol) were added sequentially via pipette. The reaction mixture was stirred for 15 min at 23° C., during which the color of the solution turned dark brown. S9 (10 mg, 0.015 mmol) was added as a solid. In 15 min, LC-MS analysis showed that the starting material had been fully consumed, giving rise to a single product with the expected m/z. The reaction mixture was partitioned between saturated aqueous sodium bicarbonate solution (5 mL) and dichloromethane (5 mL). The layers were separated, and the aqueous layer was extracted with dichloromethane (2×5 mL). The combined organic layers were dried over sodium sulfate. The dried solution was filtered, and the filtrate was concentrated. The residue was purified by column chromatography (0-30% methanol-dichloromethane, 12-g RediSep(R) Gold Rf column, Teledyne ISCO, Lincoln, NE) to afford the product as a white power (2.5 mg, 25%). HRMS (C₃₆H₃₆ClFN₆O₄+H)⁺ Calc'd: 671.2549, Found: 671.2542.

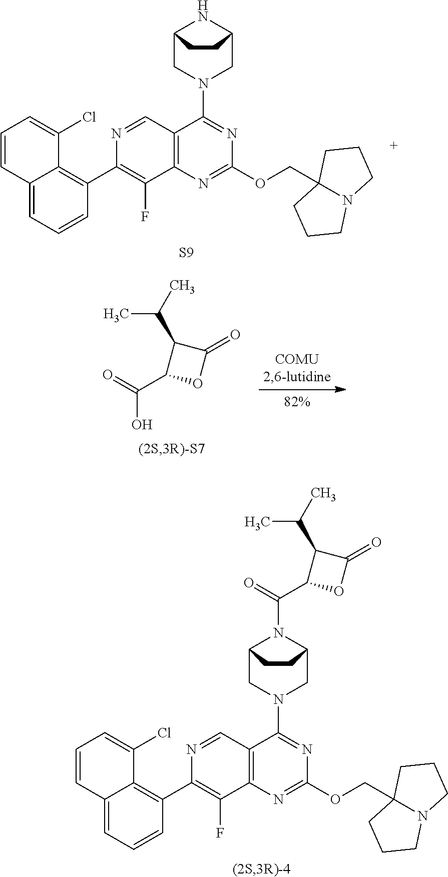

(2S,3R)-4, also referred to herein as 15-014.

An oven-dried one-dram vial was charged with (2S,3R)-3-isopropyl-4-oxo-oxetane-2-carboxylic acid (14.1 mg, 0.089 mmol), COMU (38.2 mg, 0.089 mmol) and a magnetic stir bar. DMF (0.292 mL) and 2,6-lutidine (21 μL, 0.178 mmol) were added sequentially via syringe. The reaction mixture was stirred for 15 min at 23° C., during which the color of the solution turned dark brown. 7-(8-chloro-1-naphthyl)-4-(3,8-diazabicyclo[3.2.1]octan-3-yl)-8-fluoro-2-(1,2,3,5,6,7-hexahydropyrrolizin-8-ylmethoxy)pyrido[4,3-d]pyrimidine trifluoroacetic acid salt (20 mg, 0.030 mmol) was added as a solid. In 15 min, LC-MS analysis showed full conversion to the desired product. The reaction mixture was partitioned between saturated aqueous sodium bicarbonate solution (5 mL) and dichloromethane (5 mL). The layers were separated, and the aqueous layer was extracted with dichloromethane (2×5 mL). The combined organic layers were dried over sodium sulfate. The dried solution was filtered, and the filtrate was concentrated. The residue was purified by column chromatography (0-30% methanol-dichloromethane, 12-g RediSep(R) Rf Gold column, Teledyne ISCO, Lincoln, NE) to afford the product as an off-white powder (17.1 mg, 82%). HRMS (C₃₈H₄₀ClFN₆O₄+H)⁺ Calc'd: 699.2862, Found: 699.3078.

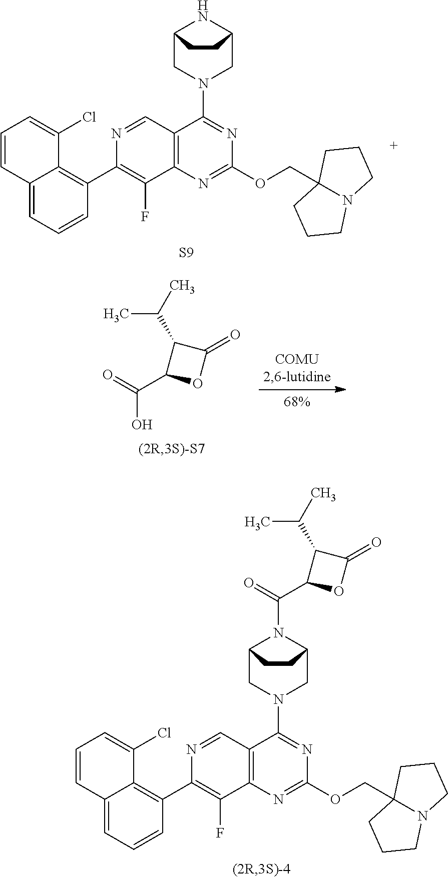

(2R,3S)-4, also referred to herein as 15-011.

An oven-dried one-dram vial was charged with (2R,3S)—S7 (7.1 mg, 0.045 mmol), COMU (19 mg, 0.045 mmol) and a magnetic stir bar. DMF (0.292 mL) and 2,6-lutidine (10 μL, 0.089 mmol) were added sequentially via syringe. The reaction mixture was stirred for 15 min at 23° C., during which the color of the solution turned dark brown. S9 (10 mg, 0.015 mmol) was added as a solid. In 15 min, LC-MS analysis showed full conversion to the desired product. The reaction mixture was partitioned between saturated aqueous sodium bicarbonate solution (5 mL) and dichloromethane (5 mL). The layers were separated, and the aqueous layer was extracted with dichloromethane (2×5 mL). The combined organic layers were dried over sodium sulfate. The dried solution was filtered, and the filtrate was concentrated. The residue was purified by column chromatography (0-30% methanol-dichloromethane, 12-g RediSep(R) Rf Gold column, Teledyne ISCO, Lincoln, NE) to afford the product as an off-white powder (7.1 mg, 68%). HRMS (C₃₈H₄₀ClFN₆O₄+H)⁺ Calc'd: 699.2862, Found: 699.2970.

### Synthesis of (2R, 3S)-3

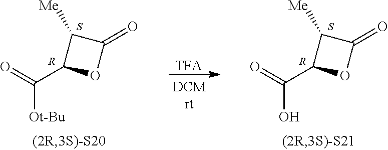

(2R,3S)-3-methyl-4-oxooxetane-2-carboxylic acid [(2R, 3S)—S21]. Precursor (2R, 3S)—S20 was synthesized using known procedure in the literature (4) using enantiopure commercial material. A 20-mL scintillation vial equipped with a stir bar was charged with (2R, 3S)—S20 (20.0 mg, 0.107 mmol). DCM (164 μL) and TFA (164 μL) were added. The resulting solution was stirred at room temperature for 30 min, at which point the consumption of the starting material was judged complete by TLC. The reaction mixture was concentrated <30° C. under reduced pressure. The residue, (2R,3S)—S21, was used in the next step without further purification.

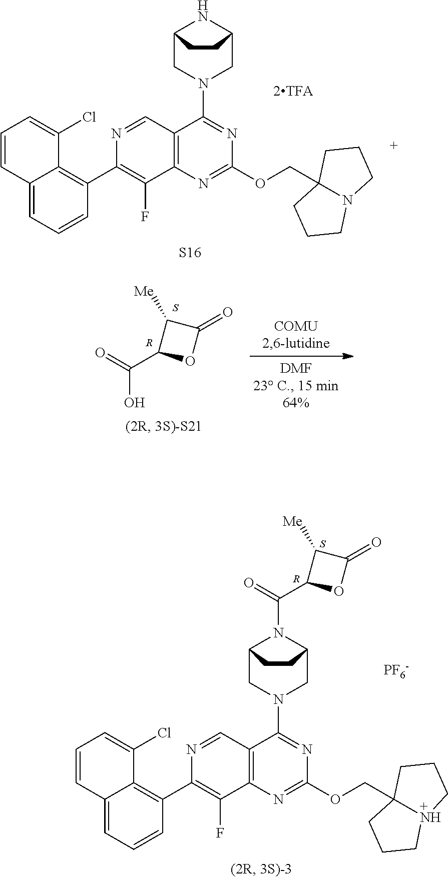

(2R,3S)-3·hexafluorophosphate. A 4-mL dram vial equipped with a stir bar was charged with (2R,3S)—S21 (11.7 mg, 0.090 mmol, 3 equiv), COMU (38.5 mg, 0.090 mmol, 3 equiv). DMF (149 μL) and 2,6-lutidine (20.9 μL, 0.189 mmol, 6 equiv) were added. The mixture was stirred at room temperature for 15 min before S16 (27.0 mg, 0.030 mmol, 1 equiv) was added as solid. The resulting solution was further stirred at room temperature for 15 min, at which point the consumption of limiting agent S16 was complete. The reaction mixture was directly loaded onto a silica gel cartridge, and purified by flash column chromatography (0-20% MeOH-DCM, 12-g Gold RediSep(R) Rf column, Teledyne ISCO, Lincoln, NE) to give the title compound as a white solid (15.3 mg, 0.019 mmol, 62% yield). ¹H NMR (600 MHz, Acetone) δ 9.27-9.16 (m, 1H), 8.21 (dd, J=8.2, 1.3 Hz, 1H), 8.10 (dd, J=8.3, 1.3 Hz, 1H), 7.74 (dd, J=8.2, 7.1 Hz, 1H), 7.65 (dd, J=7.0, 1.3 Hz, 2H), 7.58 (dd, J=8.2, 7.4 Hz, 1H), 5.30-5.15 (m, 1H), 4.91-4.62 (m, 6H), 4.10 (td, J=7.8, 4.2 Hz, 1H), 4.00-3.76 (m, 5H), 3.53-3.42 (m, 2H), 2.46-2.37 (m, 2H), 2.35-2.27 (m, 2H), 2.27-2.16 (m, 4H), 2.09 (s, 3H), 1.53-1.49 (m, 2H), 1.47 (dd, J=7.6, 1.3 Hz, 2H), 1.30 (d, J=6.3 Hz, 2H). ¹³C NMR (151 MHz, Acetone) δ 171.55, 171.50, 166.74, 164.67, 163.79, 163.57, 151.55, 149.85, 148.80, 148.72, 148.27, 148.19, 145.26, 145.22, 145.17, 145.13, 136.88, 132.52, 132.06, 131.64, 130.92, 130.33, 129.67, 129.23, 127.28, 126.74, 112.00, 82.27, 73.71, 73.49, 70.99, 56.38, 56.31, 56.28, 56.22, 55.57, 55.21, 53.09, 53.00, 50.97, 50.53, 50.51, 34.92, 34.89, 34.82, 28.21, 28.16, 28.10, 28.05, 26.12, 26.06, 25.95, 25.90, 25.19, 25.15, 25.13, 12.43, 12.34. ¹⁹F NMR (564 MHz, Acetone) δ −73.25 (d, PF₆, 6F), −141.27-−141.37 (m, 1F). Accurate MS calculated for C₃₆H₃₇ClFN₆O₄ [M+H]⁺ 671.2549, found 671.2585.

### Synthesis of (2R, 3S)-5

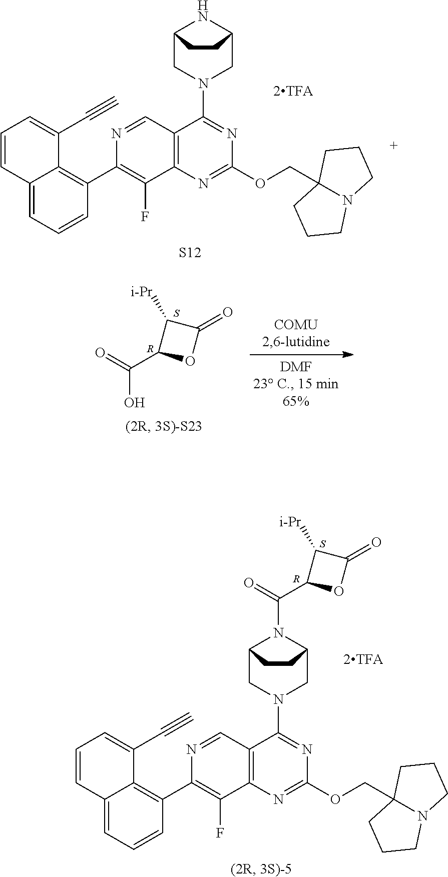

[(2R, 3S)-5]·2TFA. An oven-dried one-dram vial was charged with (2R, 3S)—S23 (6.4 mg, 0.040 mmol, 3 equiv), COMU (17.3 mg, 0.040 mmol, 3 equiv) and a magnetic stir bar. DMF (135 μL) and 2,6-lutidine (9.4 μL, 0.081 mmol, 6 equiv) were added sequentially. The reaction mixture was stirred for 15 min at room temperature. S12 (12.0 mg, 0.013 mmol) was added as a solid, and the resulting dark solution was stirred at room temperature for 15 min, at which point full consumption of S12 was judged by LC-MS. The reaction mixture was directly loaded onto purified by column chromatography (0-30% methanol-dichloromethane, 12-g RediSep(R) Rf Gold column, Teledyne ISCO, Lincoln, NE). The title compound was isolated as a red solid (29.7 mg, 0.032 mmol, 65% yield). ¹H NMR (600 MHz, Acetone) δ 12.16-11.71 (m, 2H), 9.28-9.13 (m, 1H), 8.15 (ddd, J=17.9, 8.3, 1.3 Hz, 2H), 7.78 (dd, J=7.1, 1.3 Hz, 1H), 7.72 (dd, J=8.2, 7.1 Hz, 1H), 7.65 (dd, J=7.1, 1.4 Hz, 1H), 7.58 (dd, J=8.3, 7.1 Hz, 1H), 5.39-5.26 (m, 2H, overlapping with residual MeOH), 4.91-4.72 (m, 2H, overlapping with residual MeOH), 4.01-3.90 (m, 2H), 3.91-3.74 (m, 4H), 3.38 (s, 2H), 3.26 (d, J=3.8 Hz, 1H), 2.39 (t, J=6.2 Hz, 3H), 2.34-2.15 (m, 8H), 2.00 (s, 3H), 1.33-1.22 (m, 1H), 1.22-0.98 (m, 7H). ¹³C NMR (151 MHz, Acetone) δ 170.59, 166.95, 164.71, 164.15, 163.89, 163.85, 160.25, 160.00, 159.76, 151.32, 147.76, 145.06, 144.93, 136.10, 135.37, 133.73, 131.99, 131.67, 131.64, 131.45, 131.11, 129.13, 128.62, 126.80, 126.70, 125.15, 119.90, 118.24, 116.32, 112.41, 84.62, 83.50, 81.37, 71.02, 70.91, 70.75, 64.72, 56.70, 56.48, 56.27, 55.98, 55.91, 55.73, 55.47, 55.30, 55.03, 53.49, 53.38, 53.32, 53.17, 35.45, 35.34, 28.45, 28.32, 28.16, 26.39, 26.29, 25.26, 25.23, 25.14, 20.84, 20.67, 19.93, 19.86, 19.79. ¹⁹F NMR (564 MHz, Acetone) δ −76.35 (6F, 2TFA), −140.26-−140.53 (m, 1F). Accurate MS calculated for C₄₀H₄₂FN₆O₄[M+H]⁺ 689.3252, found 689.3256.

### Synthesis of (2R, 3S)-6

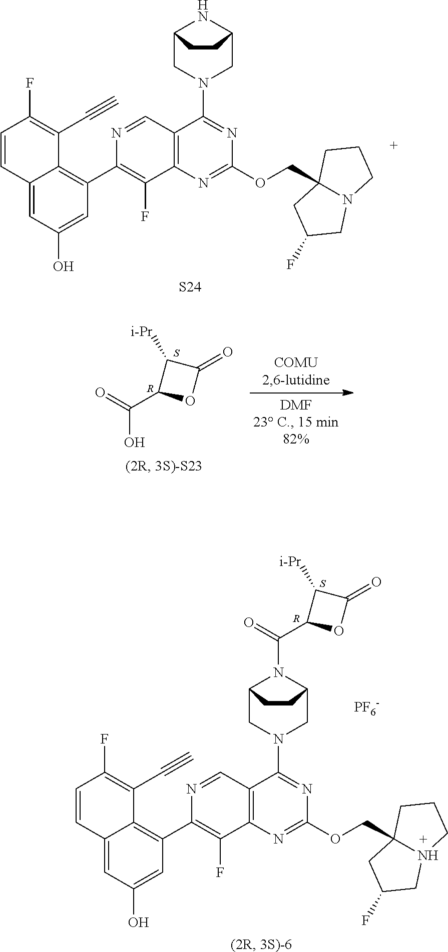

(2R, 3S)-6·hexafluorophosphate. S24 was synthesized using a known procedure (5). A 4-mL dram vial equipped with a stir bar was charged with (2R, 3S)—S23 (15.8 mg, 0.100 mmol), COMU (42.8 mg, 0.100 mmol, 3 equiv) and a magnetic stir bar. DMF (166 μL) and 2,6-lutidine (23 μL, 0.200 mmol, 6 equiv) were added sequentially via pipette. The reaction mixture was stirred for 15 min at 23° C., during which the color of the solution turned dark brown. S24 (20.0 mg, 0.033 mmol) was added as a solid, and the resulting dark solution was stirred at room temperature for 15 min, at which point full consumption of S12 was judged by LC-MS. The reaction mixture was directly loaded onto purified by column chromatography (0-30% methanol-dichloromethane, 12-g RediSep(R) Rf Gold column, Teledyne ISCO, Lincoln, NE). The title compound was isolated as an off-white solid (24.4 mg, 0.028 mmol, 83% yield). ¹H NMR (600 MHz, Acetone) δ 9.22-9.11 (m, 1H), 7.97 (dd, J=9.1, 5.8 Hz, 1H), 7.48 (dd, J=2.7, 1.2 Hz, 1H), 7.41 (t, J=9.0 Hz, 1H), 7.31 (t, J=2.4 Hz, 1H), 5.62 (s, 2H), 5.37-5.26 (m, 1H), 4.94-4.65 (m, 6H), 4.05-3.75 (m, 6H), 3.54-3.41 (m, 2H), 2.75-2.54 (m, 2H), 2.46 (d, J=11.0 Hz, 1H), 2.30 (s, 2H), 2.21 (dd, J=13.9, 7.1 Hz, 1H), 2.17-2.07 (m, 2H), 2.03-1.82 (m, 3H), 1.16-1.02 (m, 6H). ¹³C NMR (151 MHz, Acetone) δ 170.36, 170.32, 166.65, 164.69, 164.43, 164.41, 163.94, 163.68, 163.65, 162.78, 162.76, 155.07, 155.06, 152.49, 150.79, 147.94, 147.02, 146.93, 145.07, 135.03, 135.00, 133.72, 131.53, 131.46, 126.75, 123.90, 123.85, 116.92, 116.75, 112.80, 112.29, 105.15, 105.13, 105.04, 105.02, 96.65, 96.58, 95.48, 95.41, 90.48, 90.44, 90.40, 90.35, 75.93, 75.90, 71.46, 70.74, 70.64, 62.39, 62.36, 62.33, 60.57, 60.48, 60.44, 60.35, 58.31, 58.28, 56.49, 56.06, 55.69, 55.60, 55.42, 55.35, 54.93, 53.18, 53.09, 53.03, 41.42, 41.27, 41.08, 35.81, 35.65, 28.18, 28.06, 28.00, 27.88, 26.13, 26.00, 25.79, 25.78, 25.71, 25.70, 20.59, 20.42, 19.67, 19.53, 1.07. ¹⁹F NMR (376 MHz, Acetone) δ −71.64 (d, 6F, PF₆), −111.14-−111.19 (m, 1F), −141.15-−141.16 (m, 1F), −174.12-−174.46 (m, 1F). Accurate MS calculated for C₄₀H₄₀F₃N₆O₅[M+H]⁺ 741.3012, found 741.3011.

### Synthesis of (R)-7

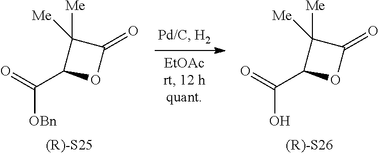 [SMILES: CC1(C)C(=O)O[C@H]1C(=O)OCc1ccccc1 ; CC1(C)C(=O)O[C@H]1C(=O)O]

(R)-3,3-dimethyl-4-oxooxetane-2-carboxylic acid [(R)—S26]. Precursor (R)—S25 was synthesized using a known method form optically pure diethyl (S)-malate (6). The enantiomeric excess (e.e.) of precursor (R)—S25 was measured by chiral HPLC 97.5%, t_{R}(R) 1.840 min [t_{R}(S) 1.704 min, CHIRALPAK IC-3 column, Gradient (B %, MeOH containing 20 mM NH₃) 5 to 20% in 4 min, hold at 20% for 2 min, temperature 25° C., Flow Rate 2 mL/min]. The absolute configuration was determined by comparing optical rotation, [σ]²⁰=−7.8 (c=0.1 g/100 mL MeOH), with reported value in the literature (6).

A 4-mL dram vial equipped with a stir bar was charged with Pd/C (2.7 mg, 10 mol %) and (R)—S25 (11.7 mg, 0.050 mmol). Ethyl acetate (1 mL) was added. The solution was bubbled with a stream of hydrogen gas for 10 min, and stirred under hydrogen atmosphere for 16 h. The reaction mixture was filtered through a plug of Celite® before concentrated to give crude (R)—S26 as a colorless oil. The crude was directly used in the next amide coupling.

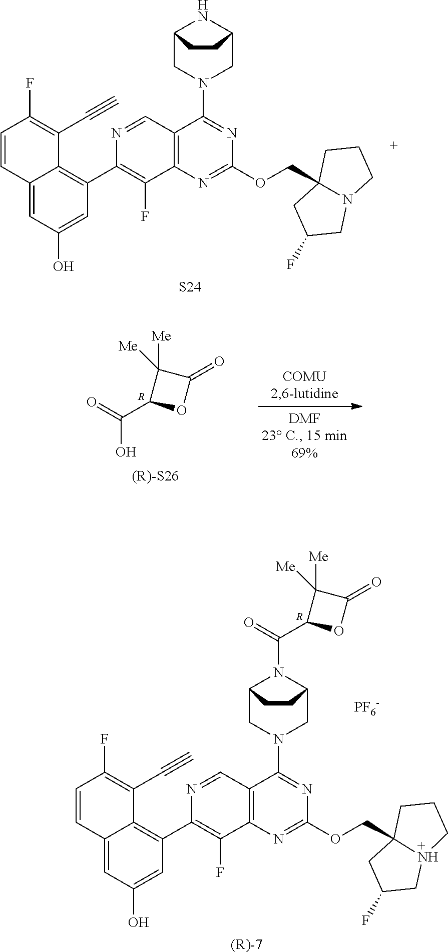

(R)-7·hexafluorophosphate. A 4-mL dram-vial was charged with crude (R)—S26, COMU (21.2 mg, 0.0500 mmol, 3 equiv). Anhydrous DMF (165 μL), and 2,6-lutidine (11.5 μL, 0.100 mmol, 6.0 equiv) were added via pipette. The reaction mixture was stirred for 15 min at 23° C., during which the color of the solution turned dark brown. Amine S24 (MRTX1133) (19.8 mg, 0.0333 mmol) was added as a solid. The mixture was further stirred at room temperature for 15 min before purified by flash column chromatography (0-30% methanol-dichloromethane, 12-g RediSep(R) Rf Gold column, Teledyne ISCO, Lincoln, NE) to give the title compound as an off-white solid (19.8 mg, 0.023 mmol, 69% yield). ¹H NMR (600 MHz, Acetone) δ 9.17-9.08 (m, 1H), 7.93 (ddd, J=9.1, 5.8, 1.9 Hz, 1H), 7.44 (t, J=2.9 Hz, 1H), 7.37 (t, J=8.9 Hz, 1H), 7.29-7.24 (m, 1H), 5.62-5.43 (m, 1H), 5.40-5.23 (m, 1H), 4.99-4.43 (m, 7H), 4.11-3.60 (m, 7H), 3.49-3.34 (m, 3H), 2.66-2.46 (m, 3H), 2.40 (t, J=8.7 Hz, 1H), 2.24 (dh, J=13.2, 6.0 Hz, 2H), 2.13-2.05 (m, 2H), 2.01 (p, J=2.2 Hz, 2H), 1.95-1.81 (m, 3H), 1.58 (d, J=9.7 Hz, 3H), 1.38 (d, J=2.4 Hz, 1H), 1.29-1.14 (m, 4H), 0.90-0.75 (m, 1H). ¹³C NMR (151 MHz, Acetone) δ 174.42, 174.25, 170.86, 166.61, 166.56, 164.64, 164.62, 164.35, 164.33, 163.08, 162.82, 162.71, 162.69, 155.02, 155.01, 152.49, 150.78, 150.73, 148.11, 148.05, 147.98, 146.92, 146.87, 146.84, 146.78, 145.12, 145.08, 144.95, 144.91, 135.01, 134.96, 133.66, 131.45, 131.39, 126.74, 126.69, 123.84, 123.80, 123.78, 116.84, 116.67, 112.71, 112.23, 112.16, 105.09, 104.98, 96.78, 96.71, 95.61, 95.54, 90.39, 90.35, 90.32, 90.28, 80.31, 78.03, 78.00, 77.15, 77.13, 75.88, 75.86, 75.83, 71.58, 60.57, 60.53, 60.50, 60.47, 60.45, 60.43, 60.40, 60.36, 60.33, 58.21, 58.18, 57.51, 57.39, 57.37, 57.00, 56.50, 56.18, 55.59, 55.34, 55.23, 55.07, 54.98, 54.89, 54.81, 52.97, 52.89, 52.86, 52.80, 41.52, 41.38, 41.33, 41.21, 35.81, 35.66, 28.54, 28.41, 28.37, 28.25, 26.42, 26.27, 25.74, 25.73, 25.66, 25.53, 25.41, 21.82, 21.76, 21.75, 20.73, 18.13, 17.62, 14.38. ¹⁹F NMR (564 MHz, Acetone) δ −72.65 (d, 6F, PF₆), −111.16-−111.24 (m, 1F), −141.02-−141.20 (m, 1F), −174.15-−174.35 (m, 1F). Accurate MS (ESI-TOF) calculated for C₃₉H₃₈F₃N₆O₅[M+H]⁺ 727.2856, found 727.2836.

### REFERENCES FOR EXAMPLE 5

1. Cammas, S., Renard, I., Boutault, K. & Gudrin, P. Tetrahedron: Asymmetry 4, 1925-1930 (1993). 2. Tello-Aburto, R., Hallada, L. P., Niroula, D. & Rogelj, S. Org. Biomol. Chem. 13, 10127-10130 (2015). 3. Wang, X. et al. WO2021041671 (2021). 4. Kawamura S, Unno Y, Asai A, Arisawa M, Shuto S. Organic & Biomolecular Chemistry. 2013; 11(38):6615-22. doi: 10.1039/C30B41338A. 5. Wang X L, Allen S, Blake J F, Bowcut V, Briere D M, Calinisan A, Dahlke J R, Fell J B, Fischer J P, Gunn R J, Hallin J, Laguer J, Lawson J D, Medwid J, Newhouse B, Nguyen P, O'Leary J M, Olson P, Pajk 5, Rahbaek L, Rodriguez M, Smith C R, Tang T P, Thomas N C, Vanderpool D, Vigers G P, Christensen J G, Marx M A. Journal of Medicinal Chemistry. 2021. doi: 10.1021/acs.jmedchem.1c01688. PubMed PMID: WOS:000731119300001. 6. Belibel R, Barbaud C. Journal of Polymer Science, Part a: Polymer Chemistry. 2015; 53(22):2586-97. doi: 10.1002/pola.27724. PubMed PMID: WOS:000362549100005. 7. Gibson, D. G.; Young, L.; Chuang, R. Y.; Venter, J. C.; Hutchison, C. A.; Smith, H. O. Nat. Methods 2009, 6 (5), 343-345. 8. Ostrem, J. M.; Peters, U.; Sos, M. L.; Wells, J. A.; Shokat, K. M. Nature 2013, 503 (7477), 548-551. 9. Gentile, D. R.; Rathinaswamy, M. K.; Jenkins, M. L.; Moss, S. M.; Siempelkamp, B. D.; Renslo, A. R.; Burke, J. E.; Shokat, K. M. Cell Chem. Biol. 2017, 24 (12), 1455-1466.e14.

### Example 6: Additional Biological Data

TABLE 4
Data represents the percentage of covalent
modification of test proteins after 24-h incubation with
100 μM compound at 23° C. at pH 7.5.

| | % covalent | % covalent | % covalent |
|---|---|---|---|
| | modification | modification | modification |
| | of wild | of K-Ras | of K-Ras |
| Compound | type K-Ras | G12D | G12E |
| 03-099 | 18.6% | 23.4% | 16.8% |
| 03-100 | 22.6% | 17.7% | 18.5% |
| 04-005 | 0% | 0% | 0% |
| 04-022 | 4.6% | 4.1% | 2.1% |
| 04-023 | 1.5% | 2.7% | 2.1% |
| 06-092 | 8.0% | 2.4% | 7.4% |
| 06-093 | 4.8% | 2.4% | 5.3% |
| 06-094 | 0% | 0% | n/a |
| 06-095 | 0% | 0% | n/a |
| 08-043 | 0% | 0% | 0% |
| 08-044 | 0% | 0% | 0% |
| 12-026 | 7.0% | 7.0% | 12.0% |
| 12-027 | 0% | 6.0% | 7.0% |
| 12-028 | 0% | 0% | 0% |
| 12-044 | 0% | 0% | 0% |
| 12-058 | 6.0% | 5.0% | 7.0% |
| 12-064 | 0% | 0% | 0% |
| 12-074 | 0% | 0% | 0% |
| 12-077 | 7.5% | 6.9% | 8.6% |
| 12-078 | n/a | n/a | n/a |
| 12-087 | 0% | 0% | 0% |
| 13-005 | 73.7% | 75.7% | 72.6% |
| 13-006 | 0% | 0% | 0% |
| 13-018 | 85.8% | 87.8% | 75.1% |
| 13-019 | 0% | 0% | 0% |
| 13-036 | 0% | 0% | 0% |
| 13-037 | 0% | 0% | 0% |
| 14-005 | 0% | 100% | 38.0% |
| 14-036 | 22.0% | 100% | 42.0% |
| (RS)-1 | 0% | 100% | 6.3% |
| (R)-2 | 0% | 100% | n/a |
| (2R, 3S)-3 | 0% | 100% | n/a |
| (2R, 3S)-4 | 0% | 100% | n/a |
| (2R, 3S)-5 | 0% | 100% | 0% |
| (2R, 3S)-6 | 0% | 100% | 0% |
| (R)-7 | 0% | 100% | 0% |

What is claimed is:

1. A compound, or a pharmaceutically acceptable salt thereof, having the formula: 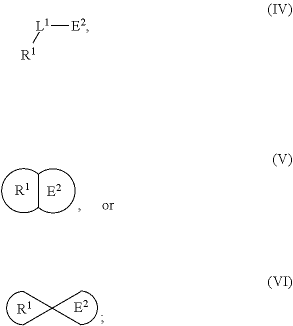
R¹ is a Switch II Binding Pocket binding moiety;
L¹ is a bond or divalent linker; and
E² is an electrophilic moiety capable of forming a covalent bond with a Switch II GTPase protein aspartate residue or a Switch II GTPase protein glutamate residue.

2. The compound of claim 1, wherein E² is an electrophilic moiety capable of forming a covalent bond with a Switch II GTPase protein aspartate residue.

3. The compound of any one of the preceding claims, wherein the Switch II GTPase protein aspartate residue is a natural Switch II GTPase protein aspartate residue.

4. The compound of any one of the preceding claims, wherein the Switch II GTPase protein aspartate residue is a mutant Switch II GTPase protein aspartate residue.

5. The compound of any one of the preceding claims, wherein the mutant Switch II GTPase protein aspartate residue is aspartate residue 12 of K-Ras(G12D), H-Ras(G12D), or N-Ras(G12D).

6. The compound of any one of the preceding claims, wherein the mutant Switch II GTPase protein aspartate residue is aspartate residue 13 of K-Ras(G13D), H-Ras(G13D), or N-Ras(G13D).

7. The compound of any one of the preceding claims, wherein E² is an electrophilic moiety capable of forming a covalent bond with a Switch II GTPase protein glutamate residue.

8. The compound of any one of the preceding claims, wherein the Switch II GTPase protein glutamate residue is a natural Switch II GTPase protein glutamate residue.

9. The compound of any one of the preceding claims, wherein the Switch II GTPase protein glutamate residue is a mutant Switch II GTPase protein glutamate residue.

10. The compound of any one of the preceding claims, wherein E² comprises a β-lactone.

11. The compound of any one of the preceding claims, wherein E² comprises a β-lactam.

12. The compound of any one of the preceding claims, having the formula:

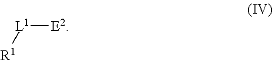

13. The compound of any one of the preceding claims, having the formula: 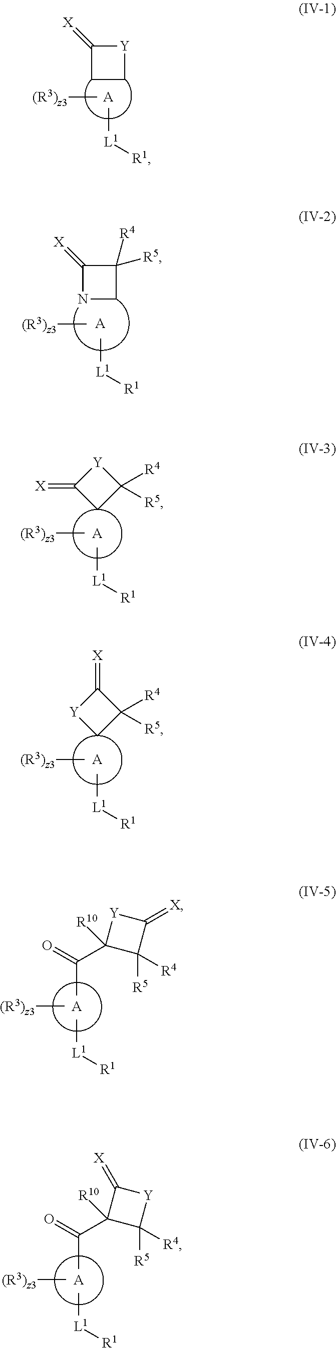 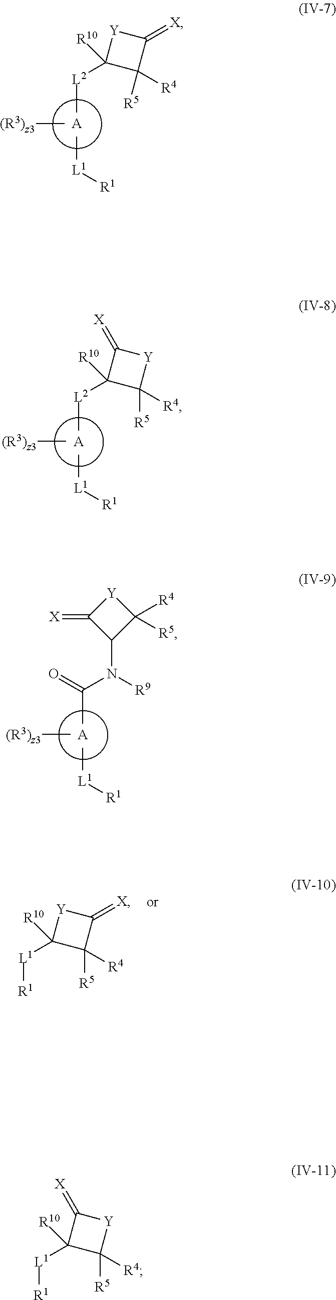
wherein
Ring A is a cycloalkyl or heterocycloalkyl;
L² is unsubstituted C₁-C₄ alkylene;
X is O or S;
Y is O, S, or NR²;
R² is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³ is independently oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R³ substituents may optionally be joined to form a substituted or unsubstituted cycloalkyl, substituted or unsubstituted heterocycloalkyl, substituted or unsubstituted aryl, or substituted or unsubstituted heteroaryl;
z3 is an integer from 0 to 10;
R⁴ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁵ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁹ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and
R¹⁰ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

14. The compound of any one of the preceding claims, wherein Ring A is a 5 to 6 membered heterocycloalkyl.

15. The compound of any one of the preceding claims, wherein Ring A is a piperidinyl, pyrrolidinyl, or piperazinyl.

16. The compound of any one of the preceding claims, having the formula:

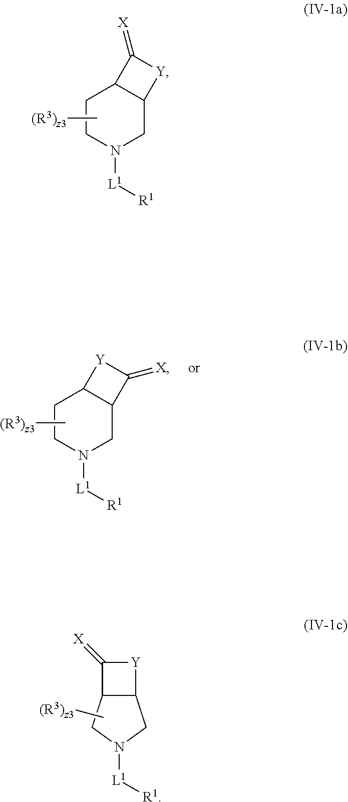

17. The compound of any one of the preceding claims, having the formula:

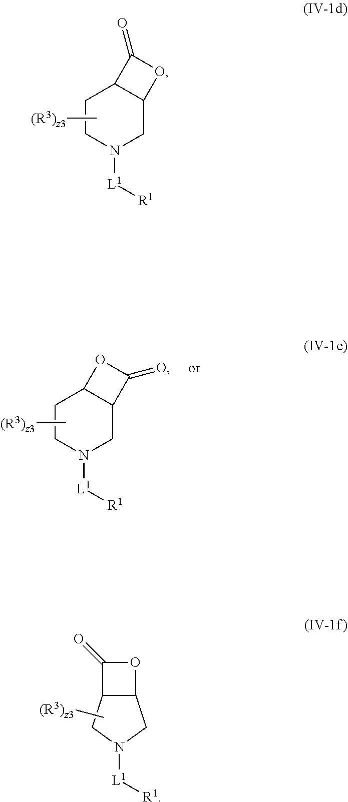

18. The compound of any one of the preceding claims, having the formula:

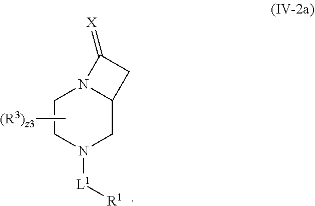

19. The compound of any one of the preceding claims, having the formula:

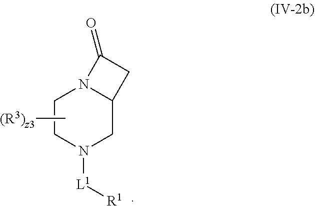

20. The compound of any one of the preceding claims, having the formula:

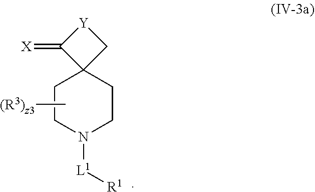

21. The compound of any one of the preceding claims, having the formula:

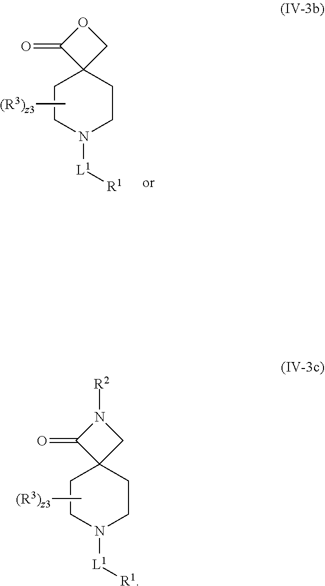

22. The compound of any one of the preceding claims, having the formula:

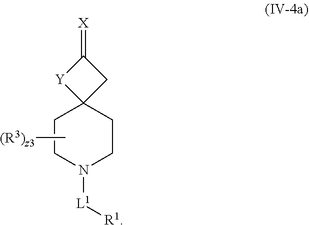

23. The compound of any one of the preceding claims, having the formula:

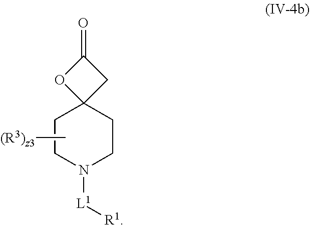

24. The compound of any one of the preceding claims, having the formula:

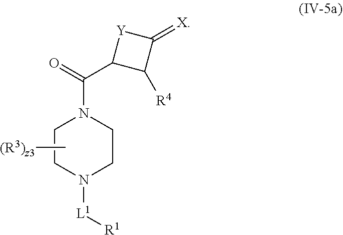

25. The compound of any one of the preceding claims, having the formula:

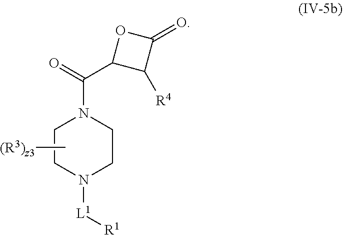

26. The compound of any one of the preceding claims, having the formula:

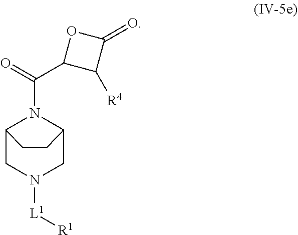

27. The compound of any one of the preceding claims, having the formula:

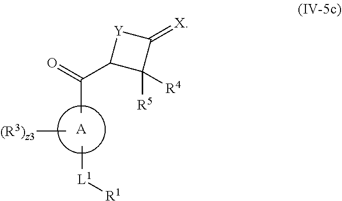

28. The compound of any one of the preceding claims, having the formula:

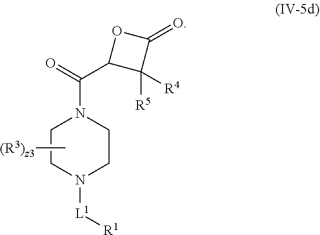

29. The compound of any one of the preceding claims, having the formula:

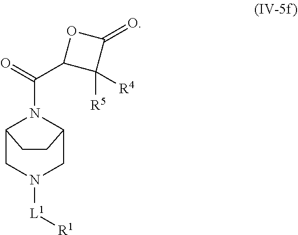

30. The compound of any one of the preceding claims, having the formula:

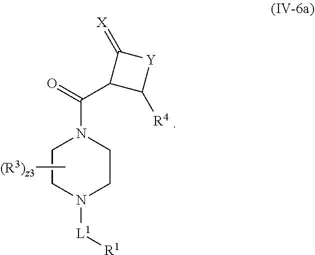

31. The compound of any one of the preceding claims, having the formula:

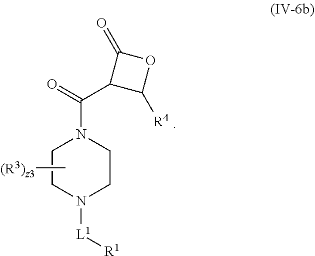

32. The compound of any one of the preceding claims, having the formula:

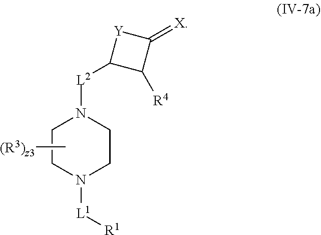

33. The compound of any one of the preceding claims, having the formula:

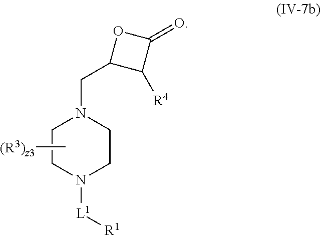

34. The compound of any one of the preceding claims, having the formula:

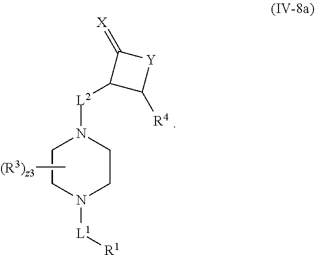

35. The compound of any one of the preceding claims, having the formula:

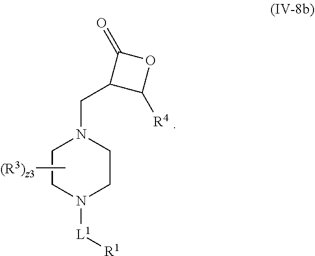

36. The compound of any one of the preceding claims, having the formula:

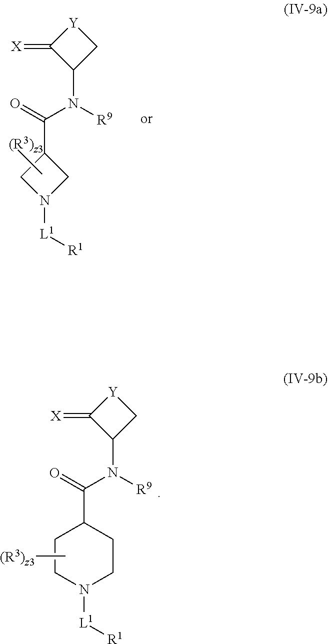

37. The compound of any one of the preceding claims, having the formula:

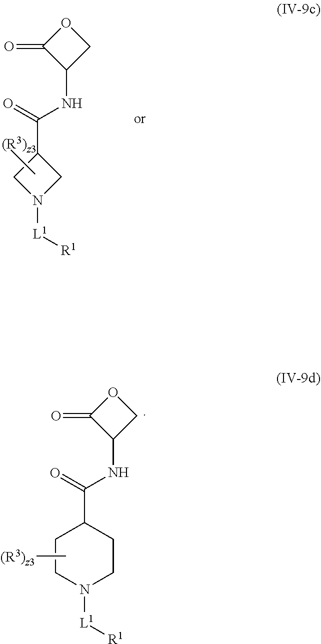

38. The compound of any one of the preceding claims, having the formula:

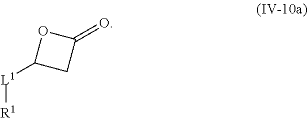

39. The compound of any one of the preceding claims, having the formula:

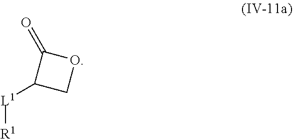

40. The compound of any one of the preceding claims, wherein R³ is independently unsubstituted C₁-C₄ alkyl.

41. The compound of any one of the preceding claims, wherein R³ is independently unsubstituted methyl.

42. The compound of any one of the preceding claims, wherein two R³ substituents are joined to form a substituted or unsubstituted cycloalkyl or substituted or unsubstituted heterocycloalkyl.

43. The compound of any one of the preceding claims, wherein
L¹ is -L¹⁰¹-L¹⁰²-L¹⁰³-;
L¹⁰¹ is connected directly to E²;
L¹⁰¹ is a bond, —C(O)—, —C(O)O—, —OC(O)—, —O—, —S—, —NR¹⁰¹—, —C(O)NR¹⁰¹—, —NR¹⁰¹C(O)—, —NR¹⁰¹C(O)O—, —OC(O)NR¹⁰¹—, —NR¹⁰¹C(O)NR¹⁰¹—, —NR¹⁰¹C(NH)NR¹⁰¹—, —S(O)₂—, —NR¹⁰¹S(O)₂—, —S(O)₂NR¹⁰¹—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L¹⁰² is a bond, —C(O)—, —C(O)O—, —OC(O)—, —O—, —S—, —NR¹⁰²—, —C(O)NR¹⁰²—, —NR¹⁰²C(O)—, —NR¹⁰²C(O)O—, —OC(O)NR¹⁰²—, —NR¹⁰²C(O)NR¹⁰²—, —NR¹⁰²C(NH)NR¹⁰²—, —S(O)₂—, —NR¹⁰²S(O)₂—, —S(O)₂NR¹⁰²—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L¹⁰³ is a bond, —C(O)—, —C(O)O—, —OC(O)—, —O—, —S—, —NR¹⁰³—, —C(O)NR¹⁰³—, —NR¹⁰³C(O)—, —NR¹⁰³C(O)O—, —OC(O)NR¹⁰³—, —NR¹⁰³C(O)NR¹⁰³—, —NR¹⁰³C(NH)NR¹⁰³—, —S(O)₂—, —NR¹⁰³S(O)₂—, —S(O)₂NR¹⁰³—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; and
each R¹⁰¹, R¹⁰², and R¹⁰³ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

44. The compound of any one of the preceding claims, wherein L¹ is a bond.

45. The compound of any one of the preceding claims, wherein L¹ is —C(O)—.

46. The compound of any one of the preceding claims, wherein L¹ is a substituted 2 to 6 membered heteroalkylene.

47. The compound of any one of the preceding claims, wherein L¹ is

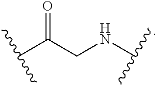 [SMILES: *NCC(*)=O]

48. The compound of any one of the preceding claims, wherein L¹ is

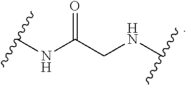 [SMILES: *NCC(=O)N*]

49. The compound of any one of the preceding claims, wherein R¹ is -L²⁰-R²⁰;
L²⁰ is a bond, —C(O)—, —C(O)O—, —OC(O)—, —O—, —S—, —NR²⁰⁰—, —C(O)NR²⁰⁰—, —NR²⁰⁰C(O)—, —NR²⁰⁰C(O)O—, —OC(O)NR²⁰⁰—, —NR²⁰⁰C(O)NR²⁰⁰—, —NR²⁰⁰C(NH)NR²⁰⁰—, —S(O)₂—, —NR²⁰⁰S(O)₂—, —S(O)₂NR²⁰⁰—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R²⁰⁰ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R²⁰ is hydrogen, halogen, —CX²⁰₃, —CHX²⁰₂, —CH₂X²⁰, —OCX²⁰₃, —OCH₂X²⁰, —OCHX²⁰₂, —CN, —SOₙ₂₀R^{20D}, —SOᵥ₂₀NR^{20A}R^{20B}, —NR^{20C}NR^{20A}R^{20B}, —ONR^{20A}R^{20B}, —NHC(O)NR^{20C}NR^{20A}R²⁰, —NHC(O)NR^{20A}R^{20B}, —N(O)ₘ₂₀, —NR^{20A}R^{20B}, —C(O)R^{20C}, —C(O)OR^{20C}, —C(O)NR^{20A}R^{20B}, —OR^{20D}, SR^{20D}, —NR^{20A}SO₂R^{20D}, —NR^{20A}C(O)R^{20C}, —NR^{20A}C(O)OR^{20C}, —NR^{20A}OR^{20C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{20A}, R^{20B}, R^{20C}, and R^{20D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{20A} and R^{20B} substituents bonded to the same nitrogen atom may optionally be joined to form a substituted or unsubstituted heterocycloalkyl or substituted or unsubstituted heteroaryl;
X²⁰ is independently —F, —Cl, —Br, or —I;
n20 is an integer from 0 to 4; and
m20 and v20 are independently 1 or 2.

50. The compound of any one of the preceding claims, wherein R¹ is 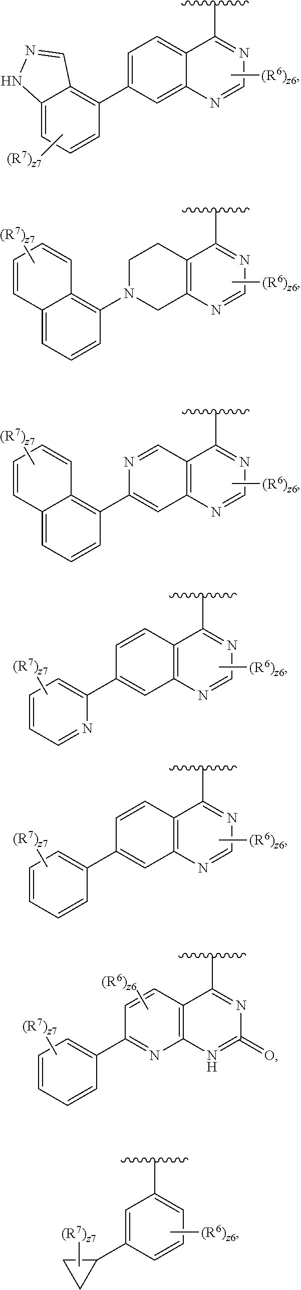 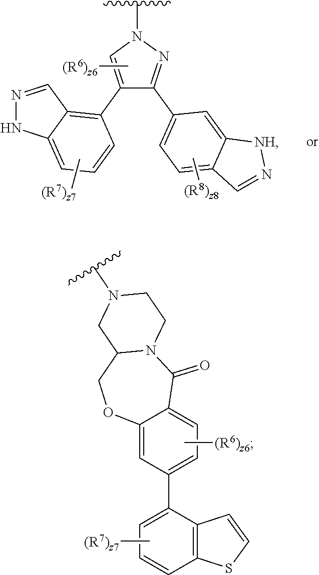
R⁶ is independently oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁷ is independently oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁸ is independently halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
z6 is an integer from 0 to 7;
z7 is an integer from 0 to 7; and
z8 is an integer from 0 to 5.

51. The compound of any one of the preceding claims, wherein R⁶ is independently a halogen, —OH, unsubstituted C₁-C₄ alkyl, substituted 2 to 6 membered heteroalkyl, or substituted 5 to 6 membered heteroaryl.

52. The compound of any one of the preceding claims, wherein R⁶ is independently —F, —Cl, —OH, or unsubstituted methyl.

53. The compound of any one of the preceding claims, wherein R⁶ is independently a 2 to 6 membered heteroalkyl, substituted with substituted heterocycloalkyl or substituted or unsubstituted fused heterocycloalkyl.

54. The compound of any one of the preceding claims, wherein R⁶ is independently

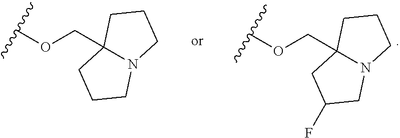 [SMILES: *OCC12CCCN1CCC2 ; *OCC12CCCN1CC(F)C2]

55. The compound of any one of the preceding claims, wherein R⁶ is independently a substituted pyridyl.

56. The compound of any one of the preceding claims, wherein z6 is 1, 2, or 3.

57. The compound of any one of the preceding claims, wherein R⁷ is independently a halogen, —CF₃, —CN, —OH, —NH₂, unsubstituted C₁-C₄ alkyl, or unsubstituted C₂-C₄ alkynyl.

58. The compound of any one of the preceding claims, wherein R⁷ is independently —F, —Cl, —CF₃, —CN, —OH, —NH₂, unsubstituted methyl, or unsubstituted ethynyl.

59. The compound of any one of the preceding claims, wherein z7 is 1, 2, or 3.

60. The compound of any one of the preceding claims, wherein R⁸ is independently a halogen or unsubstituted C₁-C₄ alkyl.

61. The compound of any one of the preceding claims, wherein R⁸ is independently —Cl or unsubstituted methyl.

62. The compound of any one of the preceding claims, wherein z8 is 1.

63. The compound of any one of the preceding claims, wherein R¹ is

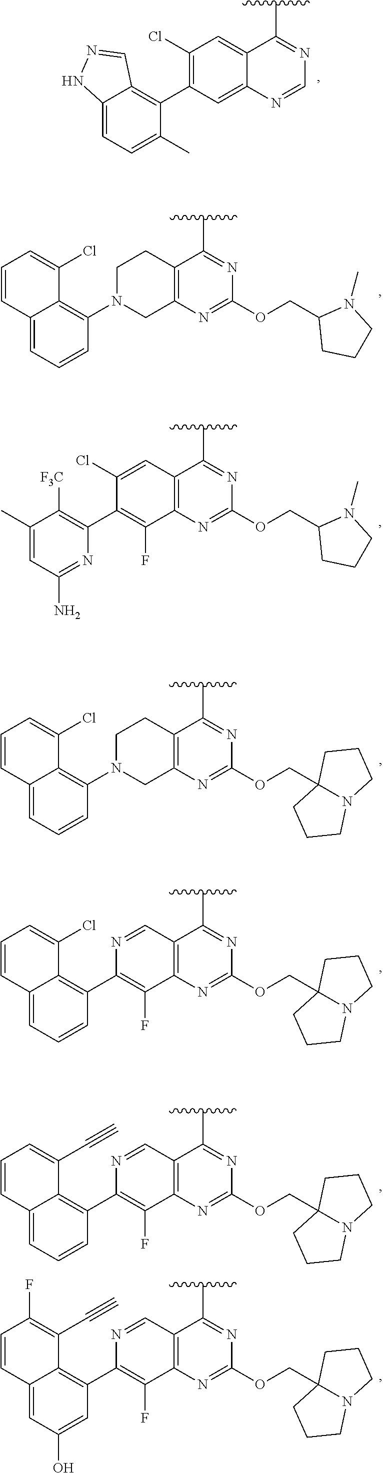

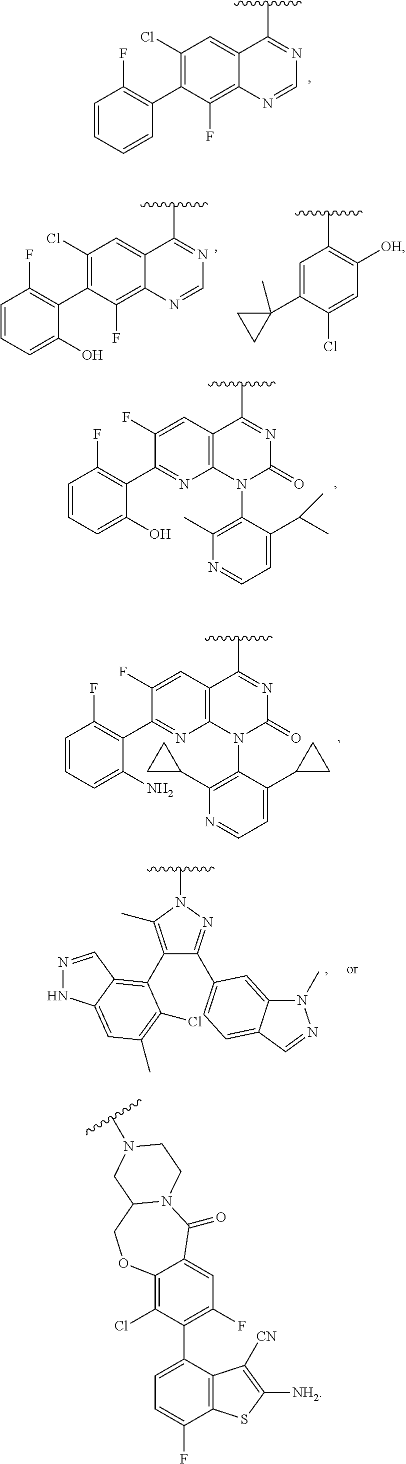

64. The compound of any one of the preceding claims, having the formula: 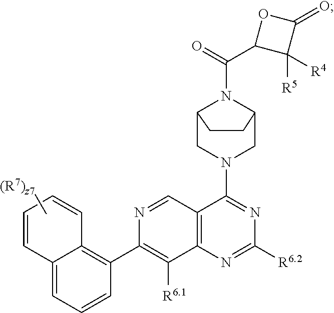
wherein
R⁴ and R⁵ are independently hydrogen or unsubstituted C₁-C₄ alkyl;
R^{6.1} is halogen;
R^{6.2} is —O—(C₁-C₄ alkyl), wherein the C₁-C₄ alkyl is substituted with a 5 to 8 membered heterocycloalkyl optionally substituted with halogen or unsubstituted C₁-C₃ alkyl;
R⁷ is independently halogen, —OH, or unsubstituted C₂ alkynyl; and
z7 is 1, 2, or 3.

65. The compound of any one of the preceding claims, having the formula:

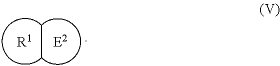

66. The compound of any one of the preceding claims, having the formula: 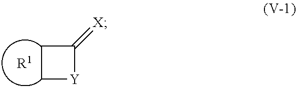
wherein
X is O or S;
Y is O, S, or NR²; and
R² is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

67. The compound of any one of the preceding claims, having the formula:

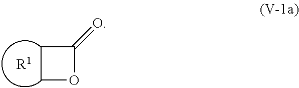

68. The compound of any one of the preceding claims, wherein R¹ is 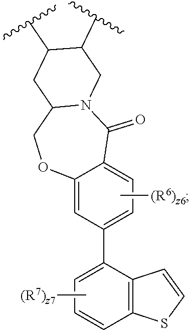
wherein
R⁶ is independently oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁷ is independently halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
z6 is an integer from 0 to 3; and
z7 is an integer from 0 to 5.

69. The compound of any one of the preceding claims, wherein R¹ is

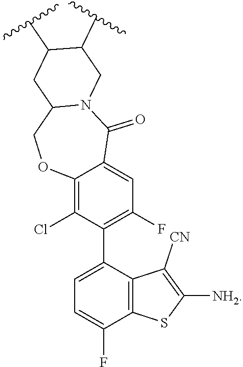 [SMILES: *C1CC2COc3c(cc(F)c(-c4ccc(F)c5sc(N)c(C#N)c45)c3Cl)C(=O)N2CC1*]

70. The compound of any one of the preceding claims, having the formula:

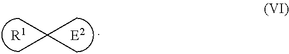

71. The compound of any one of the preceding claims, having the formula: 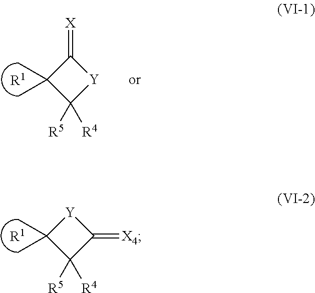
wherein
X is O or S;
Y is O, S, or NR²;
R² is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁴ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and
R⁵ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

72. The compound of any one of the preceding claims, having the formula:

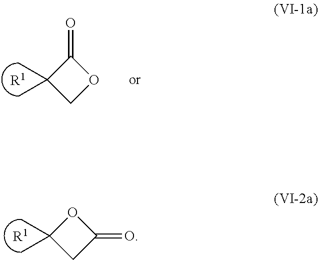

73. The compound of any one of the preceding claims, wherein R¹ is 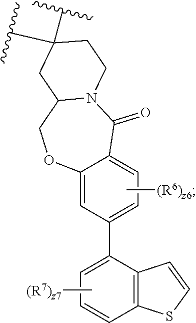
wherein
R⁶ is independently oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁷ is independently halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
z6 is an integer from 0 to 3; and
z7 is an integer from 0 to 5.

74. The compound of any one of the preceding claims, wherein R¹ is

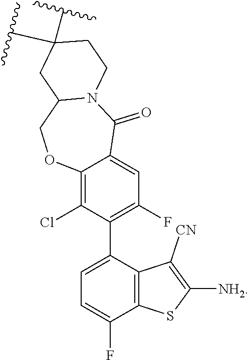 [SMILES: *C1(*)CCN2C(=O)c3cc(F)c(-c4ccc(F)c5sc(N)c(C#N)c45)c(Cl)c3OCC2C1]

75. The compound of any one of the preceding claims, having the formula:

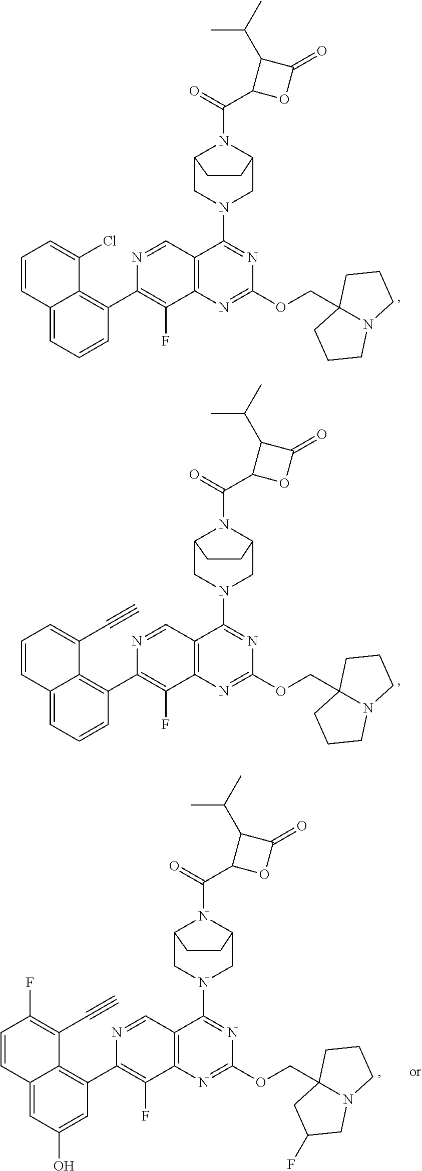 [SMILES: CC(C)C1C(=O)OC1C(=O)N1C2CCC1CN(c1nc(OCC34CCCN3CCC4)nc3c(F)c(-c4cccc5cccc(Cl)c45)ncc13)C2 ; C#Cc1cccc2cccc(-c3ncc4c(N5CC6CCC(C5)N6C(=O)C5OC(=O)C5C(C)C)nc(OCC56CCCN5CCC6)nc4c3F)c12 ; C#Cc1c(F)ccc2cc(O)cc(-c3ncc4c(N5CC6CCC(C5)N6C(=O)C5OC(=O)C5C(C)C)nc(OCC56CCCN5CC(F)C6)nc4c3F)c12]

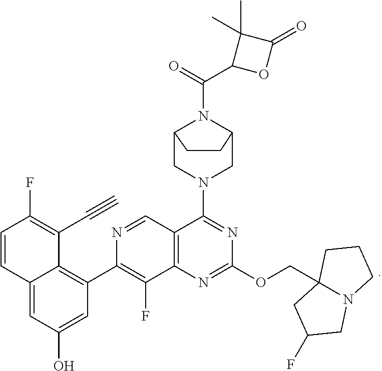 [SMILES: C#Cc1c(F)ccc2cc(O)cc(-c3ncc4c(N5CC6CCC(C5)N6C(=O)C5OC(=O)C5(C)C)nc(OCC56CCCN5CC(F)C6)nc4c3F)c12]

76. A pharmaceutical composition comprising a pharmaceutically acceptable excipient and a compound of one of claims 1 to 75, or a pharmaceutically acceptable salt thereof.

77. A method of treating cancer in a subject in need thereof, said method comprising administering to the subject in need thereof a therapeutically effective amount of a compound of one of claims 1 to 75, or a pharmaceutically acceptable salt thereof.

78. The method of claim 77, wherein the cancer is rectal carcinoma, colorectal adenocarcinoma, colorectal carcinoma, non-small cell lung carcinoma, squamous cell lung carcinoma, myelodysplastic syndrome, or acute myeloid leukemia.

79. A method of treating a K-Ras(G12D)-associated disease in a subject in need thereof, said method comprising administering to the subject in need thereof a therapeutically effective amount of a compound of one of claims 1 to 75, or a pharmaceutically acceptable salt thereof.

80. A method of treating an H-Ras(G12D)-associated disease in a subject in need thereof, said method comprising administering to the subject in need thereof a therapeutically effective amount of a compound of one of claims 1 to 75, or a pharmaceutically acceptable salt thereof.

81. A method of treating an N-Ras(G12D)-associated disease in a subject in need thereof, said method comprising administering to the subject in need thereof a therapeutically effective amount of a compound of one of claims 1 to 75, or a pharmaceutically acceptable salt thereof.

82. A method of modulating the level of activity of a K-Ras protein in a cell, said method comprising contacting the cell with an effective amount of a compound of one of claims 1 to 75, or a pharmaceutically acceptable salt thereof.

83. The method of claim 82, wherein said modulating of said activity comprises modulating GTPase activity, nucleotide exchange, differential GDP or GTP binding, effector protein binding, effector protein activation, guanine exchange factor (GEF) binding, GEF-facilitated nucleotide exchange, phosphate release, nucleotide release, nucleotide binding, K-Ras subcellular localization, K-Ras post-translational processing, or K-Ras post-translational modifications.

84. The method of one of claims 82 to 83, wherein said modulating is increasing the activity of said K-Ras protein.

85. The method of one of claims 82 to 83, wherein said modulating is reducing the activity of said K-Ras protein.

86. The method of one of claims 82 to 85, wherein said K-Ras protein is a human K-Ras protein.

87. The method of claim 86, wherein said human K-Ras protein contains a G12D mutation.

88. A method of modulating the level of activity of an H-Ras protein in a cell, said method comprising contacting the cell with an effective amount of a compound of one of claims 1 to 75, or a pharmaceutically acceptable salt thereof.

89. The method of claim 88, wherein said modulating of said activity comprises modulating GTPase activity, nucleotide exchange, differential GDP or GTP binding, effector protein binding, effector protein activation, guanine exchange factor (GEF) binding, GEF-facilitated nucleotide exchange, phosphate release, nucleotide release, nucleotide binding, H-Ras subcellular localization, H-Ras post-translational processing, or H-Ras post-translational modifications.

90. The method of one of claims 88 to 89, wherein said modulating is increasing the activity of said H-Ras protein.

91. The method of one of claims 88 to 89, wherein said modulating is reducing the activity of said H-Ras protein.

92. The method of one of claims 88 to 91, wherein said H-Ras protein is a human H-Ras protein.

93. The method of claim 92, wherein said human H-Ras protein contains a G12D mutation.

94. A method of modulating the level of activity of an N-Ras protein in a cell, said method comprising contacting the cell with an effective amount of a compound of one of claims 1 to 75, or a pharmaceutically acceptable salt thereof.

95. The method of claim 94, wherein said modulating of said activity comprises modulating GTPase activity, nucleotide exchange, differential GDP or GTP binding, effector protein binding, effector protein activation, guanine exchange factor (GEF) binding, GEF-facilitated nucleotide exchange, phosphate release, nucleotide release, nucleotide binding, N-Ras subcellular localization, N-Ras post-translational processing, or N-Ras post-translational modifications.

96. The method of one of claims 94 to 95, wherein said modulating is increasing the activity of said N-Ras protein.

97. The method of one of claims 94 to 95, wherein said modulating is reducing the activity of said N-Ras protein.

98. The method of one of claims 94 to 97, wherein said N-Ras protein is a human K-Ras protein.

99. The method of claim 98, wherein said human N-Ras protein contains a G12D mutation.

100. A K-Ras protein covalently bound to a compound of one of claims 1 to 75, or a pharmaceutically acceptable salt thereof, wherein said compound is covalently bound to an aspartate residue of said K-Ras protein.

101. The covalently modified K-Ras protein of claim 100, wherein said compound is reversibly covalently bound to an aspartate residue of said K-Ras protein.

102. The covalently modified K-Ras protein of claim 100, wherein said compound is irreversibly covalently bound to an aspartate residue of said K-Ras protein.

103. The covalently modified K-Ras protein of one of claims 100 to 102, wherein said covalently modified K-Ras protein has a modulated activity relative to a control, wherein said activity is selected from GTPase activity, nucleotide exchange, differential GDP or GTP binding, effector protein binding, effector protein activation, guanine exchange factor (GEF) binding, GEF-facilitated nucleotide exchange, phosphate release, nucleotide release, nucleotide binding, K-Ras subcellular localization, K-Ras post-translational processing, and K-Ras post-translational modifications.

104. The covalently modified K-Ras protein of one of claims 100 to 102, wherein said K-Ras protein contains a G12D mutation.

105. The covalently modified K-Ras protein of claim 104, wherein said compound is covalently bonded to aspartate residue 12.

106. An H-Ras protein covalently bound to a compound of one of claims 1 to 75, or a pharmaceutically acceptable salt thereof, wherein said compound is covalently bound to an aspartate residue of said H-Ras protein.

107. The covalently modified H-Ras protein of claim 106, wherein said compound is reversibly covalently bound to an aspartate residue of said H-Ras protein.

108. The covalently modified H-Ras protein of claim 106, wherein said compound is irreversibly covalently bound to an aspartate residue of said H-Ras protein.

109. The covalently modified H-Ras protein of one of claims 106 to 108, wherein said covalently modified H-Ras protein has a modulated activity relative to a control, wherein said activity is selected from GTPase activity, nucleotide exchange, differential GDP or GTP binding, effector protein binding, effector protein activation, guanine exchange factor (GEF) binding, GEF-facilitated nucleotide exchange, phosphate release, nucleotide release, nucleotide binding, H-Ras subcellular localization, H-Ras post-translational processing, and H-Ras post-translational modifications.

110. The covalently modified H-Ras protein of one of claims 106 to 109, wherein said H-Ras protein contains a G12D mutation.

111. The covalently modified H-Ras protein of claim 110, wherein said compound is covalently bonded to aspartate residue 12.

112. An N-Ras protein covalently bound to a compound of one of claims 1 to 75, or a pharmaceutically acceptable salt thereof, wherein said compound is covalently bound to an aspartate residue of said N-Ras protein.

113. The covalently modified N-Ras protein of claim 112, wherein said compound is reversibly covalently bound to an aspartate residue of said N-Ras protein.

114. The covalently modified N-Ras protein of claim 112, wherein said compound is irreversibly covalently bound to an aspartate residue of said N-Ras protein.

115. The covalently modified N-Ras protein of one of claims 112 to 114, wherein said covalently modified H-Ras protein has a modulated activity relative to a control, wherein said activity is selected from GTPase activity, nucleotide exchange, differential GDP or GTP binding, effector protein binding, effector protein activation, guanine exchange factor (GEF) binding, GEF-facilitated nucleotide exchange, phosphate release, nucleotide release, nucleotide binding, N-Ras subcellular localization, N-Ras post-translational processing, and N-Ras post-translational modifications.

116. The covalently modified N-Ras protein of one of claims 112 to 115, wherein said H-Ras protein contains a G12D mutation.

117. The covalently modified N-Ras protein of claim 116, wherein said compound is covalently bonded to aspartate residue 12.


SEQUENCE LISTING (56 sequences)

SEQ ID NO: 1 - protein, 189 aa, synthetic construct [sequence omitted]

SEQ ID NO: 2 - protein, 189 aa, synthetic construct
  REGION 1..189: note = Synthetic polypeptide
MTEYKLVVVGAGGVGKSALTIQLIQNHFVDEYDPTIEDSYRKQVVIDGETCLLDILDTAGQEEYSAMRDQYMRTGEGFLCVFAINNTKSFEDIHQYREQIKRVKDSDDVPMVLVGNKCDLAARTVESRQAQDLARSYGIPYIETSAKTRQGVEDAFYTLVREIRQHKLRKLNPPDESGPGCMSCKCVLS

SEQ ID NO: 3 - protein, 189 aa, synthetic construct
  REGION 1..189: note = Synthetic polypeptide
MTEYKLVVVGAGGVGKSALTIQLIQNHFVDEYDPTIEDSYRKQVVIDGETCLLDILDTAGQEEYSAMRDQYMRTGEGFLCVFAINNSKSFADINLYREQIKRVKDSDDVPMVLVGNKCDLPTRTVDTKQAHELAKSYGIPFIETSAKTRQGVEDAFYTLVREIRQYRMKKLNSSDDGTQGCMGLPCVVM

SEQ ID NO: 4 - protein, 151 aa, synthetic construct
  REGION 1..151: note = Synthetic polypeptide
MTEYKLVVVGASGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQR

SEQ ID NO: 5 - DNA, 25 bp, synthetic construct
  misc_feature 1..25: note = Synthetic polynucleotide
  source 1..25: mol_type = other DNA
actcttgcctacgccactagctcca

SEQ ID NO: 6 - protein, 104 aa, synthetic construct
  REGION 1..104: note = Synthetic polypeptide
MELPTKPGTFDLGLATWSPSFQGETHRAQARRRDVGRQLPEYKAVVVGASGVGKSALTIQLNHQCFVEDHDPTIQDSYWKELTLDSGDCILNVLDTAGQAIHRA

SEQ ID NO: 7 - protein, 87 aa, synthetic construct
  REGION 1..87: note = Synthetic polypeptide
MKLAAMIKKMCPSDSELSIPAKNCYRMVILGSSKVGKTAIVSRFLTGRFEDAYTPTIEDFHRKFYSIRGEVYQLDILDTSGNHPFPA

SEQ ID NO: 8 - protein, 82 aa, synthetic construct
  REGION 1..82: note = Synthetic polypeptide
MMKTLSSGNCTLSVPAKNSYRMVVLGASRVGKSSIVSRFLNGRFEDQYTPTIEDFHRKVYNIRGDMYQLDILDTSGNHPFPA

SEQ ID NO: 9 - protein, 98 aa, synthetic construct
  REGION 1..98: note = Synthetic polypeptide
MRLIQNMCTIAEYPAPGNAAASDCCVGAAGRRLVKIAVVGASGVGKTALVVRFLTKRFIGDYERNAGNLYTRQVQIEGETLALQVQDTPGIQVHENSL

SEQ ID NO: 10 - protein, 115 aa, synthetic construct
  REGION 1..115: note = Synthetic polypeptide
GAPRRRGSMPVPYKHQLRRAQAVDELDWPPQASSSGSSDSLGSGEAAPAQKDGIFKVMLVGESGVGKSTLAGTFGGLQGDSAHEPENPEDTYERRIMVDKEEVTLVVYDIWEQGD

SEQ ID NO: 11 - protein, 101 aa, synthetic construct
  REGION 1..101: note = Synthetic polypeptide
MLSSIKCVLVGDSAVGKTSLLVRFTSETFPEAYKPTVYENTGVDVFMDGIQISLGLWDTAGNDAFRSIRPLSYQQADVVLMCYSVANHNSFLNLKNKWIGE

SEQ ID NO: 12 - protein, 120 aa, synthetic construct
  REGION 1..120: note = Synthetic polypeptide
MKERRASQKLSSKSIMDPNQNVKCKIVVVGDSQCGKTALLHVFAKDCFPENYVPTVFENYTASFEIDTQRIELSLWDTSGSPYYDNVRPLSYPDSDAVLICFDISRPETLDSVLKKWKGE

SEQ ID NO: 13 - protein, 119 aa, synthetic construct
  REGION 1..119: note = Synthetic polypeptide
MSSMNPEYDYLFKLLLIGDSGVGKSCLLLRFADDTYTESYISTIGVDFKIRTIELDGKTIKLQIWDTAGQERFRTITSSYYRGAHGIIVVYDVTDQESFNNVKQWLQEIDRYASENVNK

SEQ ID NO: 14 - protein, 116 aa, synthetic construct
  REGION 1..116: note = Synthetic polypeptide
MNPEYDYLFKLLLIGDSGVGKSCLLLRFADDTYTESYISTIGVDFKIRTIELDGKTIKLQIWDTAGQERFRTITSSYYRGAHGIIVVYDVTDQESYANVKQWLQEIDRYASENVNK

SEQ ID NO: 15 - protein, 114 aa, synthetic construct
  REGION 1..114: note = Synthetic polypeptide
MAYAYLFKYIIIGDTGVGKSCLLLQFTDKRFQPVHDLTIGVEFGARMITIDGKQIKLQIWDTAGQESFRSITRSYYRGAAGALLVYDITRRDTFNHLTTWLEDARQHSNSNMVI

SEQ ID NO: 16 - protein, 114 aa, synthetic construct
  REGION 1..114: note = Synthetic polypeptide
MTYAYLFKYIIIGDTGVGKSCLLLQFTDKRFQPVHDLTIGVEFGARMVNIDGKQIKLQIWDTAGQESFRSITRSYYRGAAGALLVYDITRRETFNHLTSWLEDARQHSSSNMVI

SEQ ID NO: 17 - protein, 170 aa, synthetic construct
  REGION 1..170: note = Synthetic polypeptide
MGCRQSSEEKEAARRSRRIDRHLRSESQRQRREIKLLLLGTSNSGKSTIVKQMKIIHSGGFNLEACKEYKPLIIYNAIDSLTRIIRALAALRIDFHNPDRAYDAVQLFALTGPAESKGEITPELLGVMRRLWADPGAQACFSRSSEYHLEDNAAYYLNDLERIAAADYIP

SEQ ID NO: 18 - protein, 170 aa, synthetic construct
  REGION 1..170: note = Synthetic polypeptide
FLQQRLKKAVPYNRMKLMIVGNTGSGKTTLLQQLMKTKKSDLGMQSATVGIDVKDWPIQIRDKRKRDLVLNWDFAGREEFYSTHPHFMTQRALYLAVYDLSKGQAEVDAMKPWLFNIKARASSSPVILVGTHLDVSDEKQRKACMSKITKELLNKRGFPAIRDYHFVNAT

SEQ ID NO: 19 - protein, 11 aa, synthetic construct
  REGION 1..11: note = Synthetic polypeptide
  SITE 7: note = MISC_FEATURE - beta-propiolactone
LVVVGADGVGK

SEQ ID NO: 20 - protein, 40 aa, synthetic construct
  REGION 1..40: note = Synthetic polypeptide
MGNIFANLFKGLFGKKEMRILMVGLDAAGKTTILYKLKLG

SEQ ID NO: 21 - protein, 40 aa, synthetic construct
  REGION 1..40: note = Synthetic polypeptide
MGNIFGNLLKSLIGKKEMRILMVGLDAAGKTTILYKLKLG

SEQ ID NO: 22 - protein, 40 aa, synthetic construct
  REGION 1..40: note = Synthetic polypeptide
MGLTISSLFSRLFGKKQMRILMVGLDAAGKTTILYKLKLG

SEQ ID NO: 23 - protein, 40 aa, synthetic construct
  REGION 1..40: note = Synthetic polypeptide
MGLTVSALFSRIFGKKQMRILMVGLDAAGKTTILYKLKLG

SEQ ID NO: 24 - protein, 36 aa, synthetic construct
  REGION 1..36: note = Synthetic polypeptide
MGKVLSKIFGNKEMRILMLGLDAAGKTTILYKLKLG

SEQ ID NO: 25 - protein, 39 aa, synthetic construct
  REGION 1..39: note = Synthetic polypeptide
MAFTKDNRVHIGPKMEIRVVTLGLDGAGKTTILFKLKQD

SEQ ID NO: 26 - protein, 40 aa, synthetic construct
  REGION 1..40: note = Synthetic polypeptide
MGGFFSSIFSSLFGTREMRILILGLDGAGKTTILYRLQVG

SEQ ID NO: 27 - protein, 35 aa, synthetic construct
  REGION 1..35: note = Synthetic polypeptide
MGLLTILKKMKQKERELRLLMLGLDNAGKTTILKK

SEQ ID NO: 28 - protein, 36 aa, synthetic construct
  REGION 1..36: note = Synthetic polypeptide
MGLLSILRKLKSAPDQEVRILLLGLDNAGKTTLLKQ

SEQ ID NO: 29 - protein, 39 aa, synthetic construct
  REGION 1..39: note = Synthetic polypeptide
MGDQTSILSNLPSFQSFHIVILGLDCAGKTTVLYRLQFN

SEQ ID NO: 30 - protein, 39 aa, synthetic construct
  REGION 1..39: note = Synthetic polypeptide
MGDQTSILSSLPSFQSFHIVMLGLDCAGKTTVLYRLQFN

SEQ ID NO: 31 - protein, 39 aa, synthetic construct
  REGION 1..39: note = Synthetic polypeptide
MGILFTRIWRLFNHQEHKVIIVGLDNAGKTTILYQFSMN

SEQ ID NO: 32 - protein, 41 aa, synthetic construct
  REGION 1..41: note = Synthetic polypeptide
MGLLDRLSVLLGLKKKEVHVLCLGLDNSGKTTIINKLKPSN

SEQ ID NO: 33 - protein, 36 aa, synthetic construct
  REGION 1..36: note = Synthetic polypeptide
MGNISSNISAFQSLHIVMLGLDSAGKTTVLYRLKFN

SEQ ID NO: 34 - protein, 39 aa, synthetic construct
  REGION 1..39: note = Synthetic polypeptide
MGLIFAKLWSLFCNQEHKVIIVGLDNAGKTTILYQFLMN

SEQ ID NO: 35 - protein, 39 aa, synthetic construct
  REGION 1..39: note = Synthetic polypeptide
MRWKALSHPAWPEEKNKQILVLGLDGAGKTSVLHSLASN

SEQ ID NO: 36 - protein, 39 aa, synthetic construct
  REGION 1..39: note = Synthetic polypeptide
MGQLIAKLMSIFGNQEHTVIIVGLDNEGKTTILYRFLTN

SEQ ID NO: 37 - protein, 39 aa, synthetic construct
  REGION 1..39: note = Synthetic polypeptide
MGMAPTASSFLPHFQALHVVVIGLDSAGKTSLLYRLKFK

SEQ ID NO: 38 - protein, 35 aa, synthetic construct
  REGION 1..35: note = Synthetic polypeptide
MGSVNSRGHKAEAQVVMMGLDSAGKTTLLYKLKGH

SEQ ID NO: 39 - protein, 36 aa, synthetic construct
  REGION 1..36: note = Synthetic polypeptide
MGSLGSKNPQTKQAQVLLLGLDSAGKSTLLYKLKLA

SEQ ID NO: 40 - protein, 22 aa, synthetic construct
  REGION 1..22: note = Synthetic polypeptide
MCLLLGATGVGKTLLVKRLQEV

SEQ ID NO: 41 - protein, 39 aa, synthetic construct
  REGION 1..39: note = Synthetic polypeptide
MFCCGWFKRWREPVRKVTLLMVGLDNAGKTATAKGIQGE

SEQ ID NO: 42 - protein, 41 aa, synthetic construct
  REGION 1..41: note = Synthetic polypeptide
MYTLLSGLYKYMFQKDEYCILILGLDNAGKTTFLEQSKTRF

SEQ ID NO: 43 - protein, 39 aa, synthetic construct
  REGION 1..39: note = Synthetic polypeptide
MSRALCCKGPPPARPEYDLVCIGLTGSGKTSLLSKLCSE

SEQ ID NO: 44 - protein, 40 aa, synthetic construct
  REGION 1..40: note = Synthetic polypeptide
MADEEDEEPALEELEQREVLVLGLDGAGKSTFLRVLSGKP

SEQ ID NO: 45 - protein, 40 aa, synthetic construct
  REGION 1..40: note = Synthetic polypeptide
MIKLLDWFKALFWKEEMELTLVGLQYSGKTTFVNVIASGQ

SEQ ID NO: 46 - protein, 40 aa, synthetic construct
  REGION 1..40: note = Synthetic polypeptide
MLRLLDWFRSLFWKEEMELTLVGLQYSGKTTFVNVIASGQ

SEQ ID NO: 47 - protein, 39 aa, synthetic construct
  REGION 1..39: note = Synthetic polypeptide
MSFSSVPQFLGLNKKSGKLLFVGLNNTDKTILLHMIKDD

SEQ ID NO: 48 - protein, 39 aa, synthetic construct
  REGION 1..39: note = Synthetic polypeptide
MSFSSVLQFLGLYKKSGKLVFLGLDNAGKTTLLHMLKDD

SEQ ID NO: 49 - protein, 39 aa, synthetic construct
  REGION 1..39: note = Synthetic polypeptide
MSFSSVLQFLGLYKKTGKLVFLGLDNAGKTTLLHMLKDD

SEQ ID NO: 50 - protein, 7 aa, synthetic construct
  REGION 1..7: note = Synthetic polypeptide
ENLYFQG

SEQ ID NO: 51 - protein, 187 aa, synthetic construct
  REGION 1..187: note = Synthetic polypeptide
MHHHHHHSSGRENLYFQGMTEYKLVVVGAGGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQGVDDAFYTLVREIRKHKEK

SEQ ID NO: 52 - protein, 187 aa, synthetic construct
  REGION 1..187: note = Synthetic polypeptide
MHHHHHHSSGRENLYFQGMTEYKLVVVGAGGVGKSALTIQLIQNHFVDEYDPTIEDSYRKQVVIDGETSLLDILDTAGQEEYSAMRDQYMRTGEGFLLVFAINNTKSFEDIHHYREQIKRVKDSEDVPMVLVGNKSDLPSRTVDTKQAQDLARSYGIPFIETSAKTRQGVDDAFYTLVREIRKHKEK

SEQ ID NO: 53 - protein, 187 aa, synthetic construct
  REGION 1..187: note = Synthetic polypeptide
MHHHHHHSSGRENLYFQGMTEYKLVVVGASGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQGVDDAFYTLVREIRKHKEK

SEQ ID NO: 54 - protein, 187 aa, synthetic construct
  REGION 1..187: note = Synthetic polypeptide
MHHHHHHSSGRENLYFQGMTEYKLVVVGASGVGKSALTIQLIQNHFVDEYDPTIEDSYRKQVVIDGETSLLDILDTAGQEEYSAMRDQYMRTGEGFLLVFAINNTKSFEDIHHYREQIKRVKDSEDVPMVLVGNKSDLPSRTVDTKQAQDLARSYGIPFIETSAKTRQGVDDAFYTLVREIRKHKEK

SEQ ID NO: 55 - protein, 346 aa, synthetic construct
  REGION 1..346: note = Synthetic polypeptide
MHHHHHHSSGRENLYFQGDRFERLVELVTMMGDQGELPIAMALANVVPCSQWDELARVLVTLFDSRHLLYQLLWNMFSKEVELADSMQTLFRGNSLASKIMTFCFKVYGATYLQKLLDPLLRIVITSSDWQHVSFEVDPTRLEPSESLEENQRNLLQMTEKFFHAIISSSSEFPPQLRSVCHCLYQVVSQRFPQNSIGAVGSAMFLRFINPAIVSPYEAGILDKKPPPRIERGLKLMSKILQSIANHVLFTKEEHMRPFNDFVKSNFDAARRFFLDIASDCPTSDAVNHSLSFISDGNVLALHRLLWNNQEKIGQYLSSNRDHKAVGRRPFDKMATLLAYLGPPEH

SEQ ID NO: 56 - protein, 188 aa, synthetic construct [sequence omitted]